(19) United States
(12) Patent Application Publication
(10) Pub. No.: US 2023/0065562 A1
(43) Pub. Date: Mar. 2, 2023

(54) DUAL CONTROLS FOR THERAPEUTIC CELL ACTIVATION OR ELIMINATION

(71) Applicant: Bellicum Pharmaceuticals, Inc., Houston, TX (US)

(72) Inventors: Joseph Henri Bayle, Houston, TX (US); MyLinh Thi Duong, Houston, TX (US); Matthew Robert Collinson-Pautz, Houston, TX (US); Aaron Edward Foster, Houston, TX (US); David Micheal Spencer, Houston, TX (US)

(21) Appl. No.: 17/219,116

(22) Filed: Mar. 31, 2021

Related U.S. Application Data
(62) Division of application No. 15/377,776, filed on Dec. 13, 2016, now abandoned.
(60) Provisional application No. 62/267,277, filed on Dec. 14, 2015.

(51) Int. Cl. C12N 9/64; A61K 35/17; A61K 39/00; C07K 14/47; C07K 14/705; C12N 5/0783; C12N 9/12; C12N 9/90

(52) U.S. Cl. CPC C12N 9/6472; A61K 35/17; A61K 39/0005; C07K 14/4705; C07K 14/70578; C12N 5/0636; A61K 38/00; C12N 9/90; C12Y 207/11001; C12Y 304/22062; C12Y 502/01008; C07K 2319/70; C12N 9/12

(57) ABSTRACT

The technology relates in part to methods for controlling the activity or elimination of therapeutic cells using molecular switches that employ distinct heterodimerizer ligands, in conjunction with other multimeric ligands. The technology may be used, for example to activate or eliminate cells used to promote engraftment, to treat diseases or condition, or to control or modulate the activity of therapeutic cells that express chimeric antigen receptors or recombinant T cell receptors.

### RELATED APPLICATIONS

This application is a divisional of U.S. application Ser. No. 15/377,776, filed Dec. 13, 2016, which claims the benefit of priority to U.S. Provisional Patent Application No. 62/267,277, filed Dec. 14, 2015, the entire contents of which are each incorporated herein by reference.

### SEQUENCE LISTING

The instant application contains a Sequence Listing which has been submitted electronically in ASCII format and is hereby incorporated by reference in its entirety. Said ASCII copy, created on Mar. 30, 2021, is named 14562-100-999_SeqListing_Mar_2021. TXT and is 1,427,168 bytes in size.

### FIELD

The technology relates in part to methods for controlling the activity or elimination of therapeutic cells using molecular switches that employ distinct heterodimerizer ligands, in conjunction with other multimeric ligands. The technology may be used, for example to activate or eliminate cells used to promote engraftment, to treat diseases or condition, or to control or modulate the activity of therapeutic cells that express chimeric antigen receptors or recombinant T cell receptors.

### BACKGROUND

There is an increasing use of cellular therapy in which modified or unmodified cells, such as T cells, are administered to a patient. In some examples, cells are genetically engineered to express a heterologous gene, these modified cells are then administered to patients. Heterologous genes may be used to express chimeric antigen receptors (CARs), which are artificial receptors designed to convey antigen specificity to T cells without the requirement for MHC antigen presentation. They include an antigen-specific component, a transmembrane component, and an intracellular component selected to activate the T cell and provide specific immunity. CAR-expressing T cells may be used in various therapies, including cancer therapies. These treatments are used, for example, to target tumors for elimination, and to treat cancer and blood disorders, but these therapies may have negative side effects.

In some instances of therapeutic cell-induced adverse events, there is a need for rapid and near complete elimination of the therapeutic cells. Overzealous on-target effects, such as those directed against large tumor masses, can lead to cytokine storms, associated with tumor lysis syndrome (TLS), cytokine release syndrome (CRS) or macrophage activation syndrome (MAS). As a result, there is great interest in the development of a stable, reliable “suicide gene” that can eliminate transferred T cells or stem cells in the event that they trigger serious adverse events (SAEs), or become obsolete following treatment. Yet in some instances, the need for therapy may remain, and there may be a way to reduce the negative effects, while maintaining a sufficient level of therapy.

In some instances, there is a need to increase the activity of the therapeutic cell. For example, costimulating polypeptides may be used to enhance the activation of T cells, and of CAR-expressing T cells against target antigens, which would increase the potency of adoptive immunotherapy.

Thus, there is a need for controlled activation or elimination of therapeutic cells, to rapidly enhance the activity of or to remove the possible negative effects of donor cells used in cellular therapy, while retaining part or all of the beneficial effects of the therapy.

### SUMMARY

Chemical Induction of Dimerization (CID) with small molecules is an effective technology used to generate switches of protein function to alter cell physiology. A high specificity, efficient dimerizer is rimiducid (AP1903), which has two identical, protein-binding surfaces arranged tail-to-tail, each with high affinity and specificity for a mutant or variant of FKBP12: FKBP12(F36V) (FKBP12v36, Fᵥ₃₆ or Fᵥ), Attachment of one or more F_{V} domains onto one or more cell signaling molecules that normally rely on homodimerization can convert that protein to rimiducid control. Homodimerization with rimiducid is used in the context of an inducible caspase safety switch, and an inducible activation switch for cellular therapy, where costimulatory polypeptides including MyD88 and CD40 polypeptides are used to stimulate immune activity. Because both of these switches rely on the same ligand inducer, it is difficult to control both functions using these switches within the same cell. In some embodiments, a molecular switch is provided that is controlled by a distinct dimerizer ligand, based on the heterodimerizing small molecule, rapamycin, or rapamycin analogs (“rapalogs”). Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR. Provided in some embodiments of the present application are molecular switches that greatly augment the use of rapamycin, rapalogs and rimiducid as agents for therapeutic applications. In certain embodiments, the allele specificity of rimiducid is used to allow selective dimerization of Fᵥ-fusions. In other embodiments, a rapamycin or rapalog-inducible pro-apoptotic polypeptide, such as, for example, Caspase-9 or a rapamycin or rapalog-inducible costimulatory polypeptide, such as, for example, MyD88/CD40 (MC) is used in combination with a rimiducid-inducible pro-apoptotic polypeptide, such as, for example, Caspase-9, or a rimiducid-inducible chimeric stimulating polypeptide, such as, for example, iMC to produce dual-switches. These dual-switches can be used to control both cell proliferation and apoptosis selectively by administration of either of two distinct ligand inducers.

In other embodiments, a molecular switch is provided that provides the option to activate a pro-apoptotic polypeptide, such as, for example, Caspase-9, with either rimiducid, or rapamycin or a rapalog, wherein the chimeric pro-apoptotic polypeptide comprises both a rimiducid-induced switch and a rapamycin-, or rapalog-, induced switch. Including both molecular switches on the same chimeric pro-apoptotic polypeptide provides flexibility in a clinical setting, where the clinician can choose to administer the appropriate drug based on its specific pharmacological properties, or for other considerations, such as, for example, availability. These chimeric pro-apoptotic polypeptides may comprise, for example, both a FKBP12-Rapamycin-binding domain of mTOR (FRB), or an FRB variant, and an FKBP12 variant polypeptide, such as, for example, FKBP12v36. By FRB variant polypeptide is meant an FRB polypeptide that binds to a rapamycin analog (rapalog), for example, a rapalog provided in the present application. FRB variant polypeptides comprise one or more amino acid substitutions, bind to a rapalog, and may bind, or may not bind to rapamycin.

In one embodiment of the dual-switch technology, (Fwt.FRBΔC9/MC.FvFv) a homodimerizer, such as AP1903 (rimiducid), induces activation of a modified cell, and a heterodimerizer, such as rapamycin or a rapalog, activates a safety switch, causing apoptosis of the modified cell. In this embodiment, for example, a chimeric pro-apoptotic polypeptide, such as, for example, Caspase-9, comprising both an FKBP12 and an FRB, or FRB variant region (iFwtFRBC9) is expressed in a cell along with an inducible chimeric MyD88/CD40 costimulating polypeptide, that comprises MyD88 and CD40 polypeptides and at least two copies of FKBP12v36 (MC.FvFv). Upon contacting the cell with a dimerizer that binds to the Fv regions, the MC.FvFv dimerizes or multimerizes, and activates the cell. The cell may, for example, be a T cell that expresses a chimeric antigen receptor directed against a target antigen (CARζ). As a safety switch, the cell may be contacted with a heterodimerizer, such as, for example, rapamycin, or a rapalog, that binds to the FRB region on the iFwtFRBC9 polypeptide, as well as the FKBP12 region on the iFwtFRBC9 polypeptide, causing direct dimerization of the Caspase-9 polypeptide, and inducing apoptosis. (FIG. 43 (2), FIG. 57) In another mechanism, the heterodimerizer binds to the FRB region on the iFwtFRBC9 polypeptide, and the Fv region on the MC.FvFv polypeptide, causing scaffold-induced dimerization, due to the scaffold of two FKBP12v36 polypeptides on each MC.FvFv polypeptide (FIG. 43 (1)), and inducing apoptosis. By FKBP12 variant polypeptide is meant an FKBP12 polypeptide that comprises one or more amino acid substitutions and that binds to a ligand such as, for example, rimiducid, with at least 100 times, 500 times, or 1000 times more affinity than the ligand binds to the FKBP12 polypeptide region.

In another embodiment of the dual-switch technology, (FRBFwtMC/FvC9) a heterodimerizer, such as rapamycin or a rapalog, induces activation of a modified cell, and a homodimerizer, such as AP1903 activates a safety switch, causing apoptosis of the modified cell. In this embodiment, for example, a chimeric pro-apoptotic polypeptide, such as, for example, Caspase-9, comprising an Fv region (iFvC9) is expressed in a cell along with an inducible chimeric MyD88/CD40 costimulating polypeptide, that comprises MyD88 and CD40 polypeptides and both an FKBP12 and an FRB or FRB variant region (iFRBFwtMC) (MC.FvFv). Upon contacting the cell with rapamycin or a rapalog that heterodimerizes the FKBP12 and FRB regions, the iFRBFwtMC dimerizes or multimerizes, and activates the cell. The cell may, for example, be a T cell that expresses a chimeric antigen receptor directed against a target antigen (CAR). As a safety switch, the cell may be contacted with a homodimerizer, such as, for example, AP1903, that binds to the iFvC9 polypeptide, causing direct dimerization of the Caspase-9 polypeptide, and inducing apoptosis. (FIG. 57 (right)).

It yet another embodiment of the dual switch compositions and methods of the present application, dual switch apoptotic polypeptides, modified cells that express the dual switch apoptotic polypeptides, and nucleic acids that encode the dual switch apoptotic polypeptides are provided. These dual switch chimeric pro-apoptotic polypeptides allow for a choice of ligand inducer. For example, in one embodiment, modified cells are provided that expresses a FRB.FKBP_{V}.ΔC9 polypeptide, or a FKBPᵥ.FRBΔC9 polypeptide; apoptosis may be induced by contacting the modified cell with either a heterodimer, such as rapamycin or a rapalog, or the homodimer, rimiducid.

Thus, in some embodiments, modified cells are provided that comprise polynucleotides that encode dual switch chimeric pro-apoptotic polypeptides, for example, FRB.FKBP_{V}.ΔC9 polypeptide, or a FKBPv.FRBΔC9 polypeptides, wherein the FRB polypeptide region may be an FRB variant polypeptide region, such as, for example, FRB_{L}. It is understood that where FRB is denoted, such as, for example, the table of nomenclature herein, other FRB derivatives may be used, such as, for example, FRB_{L} Similarly, where polypeptides comprising FRB_{L} is provided as an example of a composition or method of the present application, it is understood that RB or FRB variants or derivatives other than FRB_{L} may be used, with the appropriate ligand, such as rapamycin or a rapalog. It is also understood that FKBP12 variants other than FKBP12v36 may be substituted for FKBP12v36, as appropriate The modified cells may further comprise polynucleotides that encode a heterologous protein such as, for example, a chimeric antigen receptor or a recombinant T cell receptor. The modified cells may further comprise polynucleotides that encode a costimulatory polypeptide, such as, for example, a polypeptide that comprises a MyD88 polypeptide region, or a truncated MyD88 polypeptide region lacking the TIR domain, or, for example, a polypeptide that comprises a MyD88 polypeptide region or a truncated MyD88 polypeptide region lacking the TIR domain and a CD40 cytoplasmic polypeptide region lacking the extracellular domain. Also provided in some embodiments are nucleic acids that comprise polynucleotides that encode dual switch chimeric pro-apoptotic polypeptides, for example, FRB.FKBPV.ΔC9 polypeptide, or a FKBPv.FRBΔC9 polypeptides, wherein the FRB polypeptide region may be an FRB variant polypeptide region, such as, for example, FRB_{L}. The nucleic acids may further comprise polynucleotides that encode a heterologous protein such as, for example, a chimeric antigen receptor or a recombinant T cell receptor. The nucleic acids may further comprise polynucleotides that encode a costimulatory polypeptide, such as, for example, a polypeptide that comprises a MyD88 polypeptide region, or a truncated MyD88 polypeptide region lacking the TIR domain, or, for example, a polypeptide that comprises a MyD88 polypeptide region or a truncated MyD88 polypeptide region lacking the TIR domain and a CD40 cytoplasmic polypeptide region lacking the extracellular domain.

In some embodiments of the present application, chimeric polypeptides are provided, wherein a first chimeric polypeptide comprises a first multimerizing region that binds to a first ligand; the first multimerizing region comprises a first ligand binding unit and a second ligand binding unit; the first ligand is a multimeric ligand comprising a first portion and a second portion; the first ligand binding unit binds to the first portion of the first ligand and does not bind significantly to the second portion of the first ligand; and the second ligand binding unit binds to the second portion of the first ligand and does not bind significantly to the first portion of the first ligand. In some embodiments, a second chimeric polypeptide is also provided, wherein the second chimeric polypeptide comprises a second multimerizing region that binds to a second ligand; the second multimerizing region comprises a third ligand binding unit; the second ligand is a multimeric ligand comprising a third portion; and the third ligand binding unit binds to the third portion of the second ligand and does not bind significantly to the second portion of the first ligand. Examples of first ligand binding units include, but are not limited to, FKBP12 multimerizing regions, or variants, such as FKBP12v36, examples of second ligand binding units are, for example, FRB or FRB variant multimerizing regions. Examples of a third ligand binding unit include, for example, but are not limited to, FKBP12 multimerizing regions, or variants, such as FKBP12v36. In certain embodiments, the first ligand binding unit is FKBP12, and the third ligand binding unit is FKBP12v36. In certain embodiments, the first ligand is rapamycin, or a rapalog, and the second ligand is rimiducid (AP1903).

The multimerizing regions, such as FKBP12/FRB, FRB/FKBP12, and FKBP12v36, may be located amino terminal to the pro-apoptotic polypeptide or costimulatory polypeptide, or, in other examples, may be located carboxyl terminal to the pro-apoptotic polypeptide or costimulatory polypeptide. Additional polypeptides, such as, for example, linker polypeptides, stem polypeptides, spacer polypeptides, or in some examples, marker polypeptides, may be located between the multimerizing region and the pro-apoptotic polypeptide or costimulatory polypeptide, in the chimeric polypeptides.

Thus, provided in some embodiments are modified cells, comprising a first polynucleotide encoding a chimeric pro-apoptotic polypeptide, wherein the chimeric pro-apoptotic polypeptide comprises (i) a pro-apoptotic polypeptide region; (ii) a FKBP12-Rapamycin-Binding (FRB) domain polypeptide, or FRB variant polypeptide region; and (iii) a FKBP12 or FKBP12 variant polypeptide region (FKBP12v); and a second polynucleotide encoding a chimeric costimulating polypeptide, wherein the chimeric costimulating polypeptide comprises one or more, for example, 1, 2, or 3 FKBP12 variant polypeptide regions and i) a MyD88 polypeptide region or a truncated MyD88 polypeptide region lacking the TIR domain; or ii) a MyD88 polypeptide region or a truncated MyD88 polypeptide region lacking the TIR domain, and a CD40 cytoplasmic polypeptide region lacking the CD40 extracellular domain. In some embodiments, the modified cell further comprises a third polynucleotide encoding a chimeric antigen receptor or a recombinant T cell receptor.

Also provided in some embodiments is a nucleic acid comprising a promoter operably linked to a first polynucleotide encoding a chimeric pro-apoptotic polypeptide, wherein the chimeric pro-apoptotic polypeptide comprises (i) a pro-apoptotic polypeptide region; (ii) a FKBP12-Rapamycin-Binding (FRB) domain polypeptide, or FRB variant polypeptide region; and (iii) a FKBP12 or FKBP12 variant polypeptide region (FKBP12v); and a second polynucleotide encoding a chimeric costimulating polypeptide, wherein the chimeric costimulating polypeptide comprises one or more, for example, 1, 2, or 3 FKBP12 variant polypeptide regions and i) a MyD88 polypeptide region or a truncated MyD88 polypeptide region lacking the TIR domain; or ii) a MyD88 polypeptide region or a truncated MyD88 polypeptide region lacking the TIR domain, and a CD40 cytoplasmic polypeptide region lacking the CD40 extracellular domain. In some embodiments, the chimeric costimulating polypeptide comprises a truncated MyD88 polypeptide region lacking the TIR domain and a CD40 cytoplasmic polypeptide region lacking the CD40 extracellular domain. In some embodiments, the promoter is operably linked to a third polynucleotide, wherein the third polynucleotide encodes a chimeric antigen receptor or a recombinant T cell receptor. In some embodiments, the pro-apoptotic polypeptide is a Caspase-9 polypeptide, wherein the Caspase-9 polypeptide lacks the CARD domain. In some embodiments, the cell is a T cell, tumor infiltrating lymphocyte, NK-T cell, or NK cell. Also provided in some embodiments are kits or compositions comprising nucleic acid comprising a first polynucleotide encoding a chimeric pro-apoptotic polypeptide, wherein the chimeric pro-apoptotic polypeptide comprises (i) a pro-apoptotic polypeptide region; (ii) a FKBP12-Rapamycin-Binding (FRB) domain polypeptide region, or variant thereof; and (iii) a FKBP12 polypeptide or FKBP12 variant polypeptide region (FKBP12v); and a second polynucleotide encoding a chimeric costimulating polypeptide, wherein the chimeric costimulating polypeptide comprises one or more, for example, 1, 2, or 3 FKBP12 variant polypeptide regions and i) a MyD88 polypeptide region or a truncated MyD88 polypeptide region lacking the TIR domain; or ii) a MyD88 polypeptide region or a truncated MyD88 polypeptide region lacking the TIR domain, and a CD40 cytoplasmic polypeptide region lacking the CD40 extracellular domain. In some embodiments, methods are provided for expressing a chimeric pro-apoptotic polypeptide, wherein the chimeric pro-apoptotic polypeptide comprises a pro-apoptotic polypeptide region; a FRB polypeptide or FRB variant polypeptide region; and a FKBP12 polypeptide region of the present embodiments, comprising contacting a nucleic acid of the present embodiments with a cell under conditions in which the nucleic acid is incorporated into the cell, whereby the cell expresses the chimeric pro-apoptotic polypeptide from the incorporated nucleic acid.

In some embodiments, methods are provided for stimulating an immune response in a subject, comprising: transplanting modified cells of the present embodiments into the subject, and after (a), administering an effective amount of a ligand that binds to the FKBP12 variant polypeptide region of the chimeric costimulating polypeptide to stimulate a cell mediated immune response. In some embodiments, methods are provided for administering a ligand to a subject who has undergone cell therapy using modified cells, comprising administering a ligand that binds to the FKBP variant region of the chimeric costimulating polypeptide to the human subject, wherein the modified cells comprise modified cells of the present embodiments the present embodiments. Also provided are methods for treating a subject having a disease or condition associated with an elevated expression of a target antigen expressed by a target cell, comprising a) transplanting an effective amount of modified cells into the subject; wherein the modified cells comprise a modified cell of the present embodiments, wherein the modified cell comprises a chimeric antigen receptor or a recombinant T cell receptor comprising an antigen recognition moiety that binds to the target antigen, and b) after a), administering an effective amount of a ligand that binds to the FKBP12 variant polypeptide region of the chimeric costimulating polypeptide to reduce the number or concentration of target antigen or target cells in the subject. Also provided are methods for reducing the size of a tumor in a subject, comprising a) administering a modified cell of the present embodiments to the subject, wherein the cell comprises a chimeric antigen receptor or a recombinant T cell receptor comprising an antigen recognition moiety that binds to an antigen on the tumor; and b) after a), administering an effective amount of a ligand that binds to the FKBP12 variant polypeptide region of the chimeric costimulating polypeptide to reduce the size of the tumor in the subject. Also provided are methods for controlling survival of transplanted modified cells in a subject, comprising transplanting modified cells of the present embodiments into the subject; and administering to the subject rapamycin or a rapalog that binds to the FRB polypeptide or FRB variant polypeptide region of the chimeric pro-apoptotic polypeptide in an amount effective to kill at least 30% of the modified cells that express the chimeric pro-apoptotic polypeptide.

In other embodiments, modified cells are provided comprising a first polynucleotide encoding a chimeric pro-apoptotic polypeptide, wherein the chimeric pro-apoptotic polypeptide comprises i) a pro-apoptotic polypeptide region; and ii) a FKBP12 variant polypeptide region; and a second polynucleotide encoding a chimeric costimulating polypeptide, wherein the chimeric costimulating polypeptide comprises a FKBP12-Rapamycin Binding (FRB) domain polypeptide or FRB variant polypeptide region; a FKBP12 polypeptide or FKBP12 variant polypeptide region; and a MyD88 polypeptide region or a truncated MyD88 polypeptide region lacking the TIR domain, or a MyD88 polypeptide region, or a truncated MyD88 polypeptide region lacking the TIR domain and a CD40 cytoplasmic polypeptide region lacking the CD40 extracellular domain. In some embodiments, the chimeric costimulating polypeptide comprises a truncated MyD88 polypeptide region lacking the TIR domain and a CD40 cytoplasmic polypeptide region lacking the CD40 extracellular domain. In some embodiments, the cell further comprises a third polynucleotide, wherein the third polynucleotide encodes a chimeric antigen receptor or a recombinant T cell receptor.

In some embodiments, nucleic acids are provided, wherein the nucleic acids comprise a promoter operably linked to a first polynucleotide encoding a chimeric pro-apoptotic polypeptide, wherein the chimeric pro-apoptotic polypeptide comprises i) a pro-apoptotic polypeptide region; and i) a FKBP12 variant polypeptide region; and a second polynucleotide encoding a chimeric costimulating polypeptide, wherein the chimeric costimulating polypeptide comprises i) a FKBP12-Rapamycin Binding (FRB) domain polypeptide or FRB variant polypeptide region; ii) a FKBP12 polypeptide region; and ii) a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain. In some embodiments, the chimeric costimulating polypeptide comprises a truncated MyD88 polypeptide region lacking the TIR domain and a CD40 cytoplasmic polypeptide region lacking the CD40 extracellular domain. In some embodiments, the promoter is operably linked to a third polynucleotide, wherein the third polynucleotide encodes chimeric antigen receptor or a recombinant T cell receptor. In some embodiments, the pro-apoptotic polypeptide is a Caspase-9 polypeptide, wherein the Caspase-9 polypeptide lacks the CARD domain. In some embodiments, the cell is a T cell, tumor infiltrating lymphocyte, NK-T cell, or NK cell. Also provided are kits or compositions comprising nucleic acids comprising polynucleotides of the present embodiments. Also provided are methods for expressing a chimeric pro-apoptotic polypeptide and a chimeric costimulating polypeptide, wherein a) the chimeric pro-apoptotic polypeptide comprises i) a pro-apoptotic polypeptide region; and ii) a FKBP12 variant polypeptide region; and b) the chimeric costimulating polypeptide comprises a FRB or FRB variant polypeptide region; a FKBP12 polypeptide region; and a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain comprising contacting a nucleic acid is a nucleic acid comprising a promoter operably linked to a polynucleotide coding for a chimeric pro-apoptotic polypeptide, wherein the chimeric pro-apoptotic polypeptide comprises a) a pro-apoptotic polypeptide region; b) a FKBP12-Rapamycin binding domain (FRB) polypeptide or FRB variant polypeptide region; and c) a FKBP12 variant polypeptide region, with a cell under conditions in which the nucleic acid is incorporated into the cell, whereby the cell expresses the chimeric pro-apoptotic polypeptide and the chimeric costimulating polypeptide from the incorporated nucleic acid.

In some embodiments, methods are provided of stimulating an immune response in a subject, comprising: a) transplanting modified cells of the present embodiments into the subject, and b) after (a), administering an effective amount of a rapamycin or a rapalog that binds to the FRB polypeptide or FRB variant polypeptide region of the chimeric stimulating polypeptide to stimulate a cell mediated immune response. In some embodiments, methods are provided of administering a ligand to a subject who has undergone cell therapy using modified cells, comprising administering rapamycin or a rapalog to the subject, wherein the modified cells comprise modified cells of the present embodiments. In some embodiments, methods are provided for treating a subject having a disease or condition associated with an elevated expression of a target antigen expressed by a target cell, comprising a) transplanting an effective amount of modified cells into the subject; wherein the modified cells comprise a modified cell of the present embodiments, wherein the modified cell comprises a chimeric antigen receptor or a recombinant T cell receptor comprising an antigen recognition moiety that binds to the target antigen, and b) after a), administering an effective amount of rapamycin or a rapalog that binds to the FRB polypeptide or FRB variant region of the chimeric stimulating polypeptide to reduce the number or concentration of target antigen or target cells in the subject. In some embodiments, methods are provided for reducing the size of a tumor in a subject, comprising a) administering a modified cell of the present embodiments to the subject, wherein the cell comprises a chimeric antigen receptor or a recombinant T cell receptor comprising an antigen recognition moiety that binds to an antigen on the tumor; and b) after a), administering an effective amount of rapamycin or a rapalog that binds to the FRB or FRB variant polypeptide region of the chimeric stimulating polypeptide to reduce the size of the tumor in the subject. In some embodiments, methods are provided for controlling survival of transplanted modified cells in a subject, comprising a) transplanting modified cells of the present embodiments into the subject, and after (a), administering to the subject a ligand that binds to the FKBP12 variant polypeptide region of the chimeric pro-apoptotic polypeptide in an amount effective to kill at least 90% of the modified cells that express the chimeric pro-apoptotic polypeptide.

In some embodiments of the present application, the chimeric costimulating polypeptide comprises two FKBP12 variant polypeptide regions, and a truncated MyD88 polypeptide region lacking the TIR domain. In some embodiments, the chimeric costimulating polypeptide further comprises a CD40 cytoplasmic polypeptide region lacking the CD40 extracellular domain. In some embodiments of the present application, the chimeric costimulating polypeptide comprises 2 FKBP12 variant polypeptide regions.

Also provided in the present application is a nucleic acid comprising a promoter operably linked to a polynucleotide coding for a chimeric pro-apoptotic polypeptide, wherein the chimeric pro-apoptotic polypeptide comprises a) a pro-apoptotic polypeptide region; b) a FKBP12-Rapamycin binding domain (FRB) polypeptide or FRB variant polypeptide region; and c) a FKBP12 variant polypeptide region. In some embodiments, wherein the FKBP12 variant comprises an amino acid substitution at amino acid residue 36. In some embodiments, the FKBP12 variant polypeptide region is a FKBP12v36 polypeptide region. In some embodiments, the FRB variant polypeptide region is selected from the group consisting of KLW (T2098L) (FRBL), KTF (W2101F), and KLF (T2098L, W2101F). In some embodiments, a chimeric pro-apoptotic polypeptide encoded by a nucleic acid of the present embodiments is provided. In some embodiments, modified cells are provided that are transfected or transduced with a nucleic acid of the present embodiments. In some embodiments, the modified cells comprise a polynucleotide that encodes a chimeric antigen receptor or a recombinant TCR. In some embodiments, methods are provided of controlling survival of transplanted modified cells in a subject, comprising: a) transplanting modified cells of the present embodiments, wherein the modified cells comprise a nucleic acid comprising a promoter operably linked to a polynucleotide coding for a chimeric pro-apoptotic polypeptide, wherein the chimeric pro-apoptotic polypeptide comprises a) a pro-apoptotic polypeptide region; b) a FKBP12-Rapamycin binding domain (FRB) polypeptide or FRB variant polypeptide region; and c) a FKBP12 variant polypeptide region. of the present embodiments into the subject; and b) after (a), administering to the subject i) a first ligand that binds to the FRB or FRB variant polypeptide region of the chimeric pro-apoptotic polypeptide; or ii) a second ligand that binds to the FKBP12 variant polypeptide region of the chimeric pro-apoptotic polypeptide wherein the first ligand or the second ligand are administered in an amount effective to kill at least 30% of the modified cells that express the chimeric pro-apoptotic polypeptide.

Autologous T cells expressing chimeric antigen receptors (CARs) directed toward tumor-associated antigens (TAAs) have had a transformational effect in initial clinical trials on the treatment of certain types of leukemias (“liquid tumors”) and lymphomas with objective response (OR) rates approaching 90%. Despite their great clinical promise and the predictable accompanying enthusiasm, this success is tempered by the observed high level of on-target, off-tumor adverse events, typical of a cytokine release syndrome (CRS). To maintain the benefit of these revolutionary treatments while minimizing the risk, a tunable safety switch has been developed, in order to control the activity level of CAR-expressing T cells. An inducible costimulatory chimeric polypeptide allows for a sustained, modulated control of a chimeric antigen receptor (CAR) that is co-expressed in the cell. The ligand inducer activates the CAR-expressing cell by multimerizing the inducible chimeric signaling molecules, which, in turn, induces NF-κB and other intracellular signaling pathways, leading to the activation of the target cells, for example, a T cell, a tumor-infiltrating lymphocyte (TIL), a natural killer (NK) cell, or a natural killer T (NK-T) cell. In the absence of the ligand inducer, the T cell is quiescent, or has a basal level of activity.

At the second level of control, a “dimmer” switch may allow for continued cell therapy, while reducing or eliminating significant side effects by eliminating the therapeutic cells from the subject, as needed. This dimmer switch is dependent on a second ligand inducer. In some examples, where there is a need to rapidly eliminate the therapeutic cells, an appropriate dose of the second ligand inducer is administered in order to eliminate over 90% or 95% of the therapeutic cells from the patient. This second level of control may be “tunable,” that is, the level of removal of the therapeutic cells may be controlled so that it results in partial removal of the therapeutic cells. This second level of control may include, for example, a chimeric pro-apoptotic polypeptide.

In some examples, the chimeric apoptotic polypeptide comprises a binding site for rapamycin, or a rapamycin analog (rapalog); also present in the therapeutic cell is an inducible chimeric polypeptide that, upon induction by a ligand inducer, activates the therapeutic cell; in some examples, the inducible chimeric polypeptide provides costimulatory activity to the therapeutic cell. The CAR may be present on a separate polypeptide expressed in the cell. In other examples, the CAR may be present as part of the same polypeptide as the inducible chimeric polypeptide. Using this controllable first level, the need for continued therapy, or the need to stimulate therapy, may be balanced with the need to eliminate or reduce the level of negative side effects.

In some embodiments, a rapamycin analog, or “rapalog”, is administered to the patient, which then binds to both the caspase polypeptide and the chimeric antigen receptor, thus recruiting the caspase polypeptide to the location of the CAR, and aggregating the caspase polypeptide. Upon aggregation, the caspase polypeptide induces apoptosis. The amount of rapamycin or rapamycin analog administered to the patient may vary; if the removal of a lower level of cells by apoptosis is desired in order to reduce side effects and continue CAR therapy, a lower level of rapamycin or rapalog may be administered to the patient.

At the second level of therapeutic cell elimination, selective apoptosis may be induced in cells that express a chimeric Caspase-9 polypeptide fused to a dimeric ligand binding polypeptide, such as, for example, the AP1903-binding polypeptide FKBP12v36, by administering rimiducid (AP1903). In some examples, the Caspase-9 polypeptide includes amino acid substitutions that result in a lower level of basal apoptotic activity as part of the inducible chimeric polypeptide, than the wild type Caspase-9 polypeptide.

In some embodiments, the nucleic acid encoding the chimeric polypeptides of the present application further comprise a polynucleotide encoding a chimeric antigen receptor, a T cell receptor, or a T cell receptor-based chimeric antigen receptor. In some embodiments, the chimeric antigen receptor comprises (i) a transmembrane region, (ii) a T cell activation molecule, and (iii) an antigen recognition moiety. Also provided are modified cells transfected or transduced with a nucleic acid discussed herein.

In some aspects of the present application, the cells are transduced or transfected with a viral vector. The viral vector may be, for example, but not limited to, a retroviral vector, such as, for example, but not limited to, a murine leukemia virus vector; an SFG vector; and adenoviral vector, or a lentiviral vector.

In some embodiments, the cell is isolated. In some embodiments, the cell is in a human subject. In some embodiments, the cell is transplanted in a human subject.

In some embodiments, personalized treatment is provided wherein the stage or level of the disease or condition is determined before administration of the multimeric ligand, before the administration of an additional dose of the multimeric ligand, or in determining method and dosage involved in the administration of the multimeric ligand. These methods may be used in any of the methods of any of the diseases or conditions of the present application. Where these methods of assessing the patient before administering the ligand are discussed in the context of graft versus host disease, it is understood that these methods may be similarly applied to the treatment of other conditions and diseases. Thus, for example, in some embodiments of the present application, the method comprises administering therapeutic cells to a patient, and further comprises identifying a presence or absence of a condition in the patient that requires the removal of transfected or transduced therapeutic cells from the patient; and administering a multimeric ligand that binds to the multimerizing region, maintaining a subsequent dosage of the multimeric ligand, or adjusting a subsequent dosage of the multimeric ligand to the patient based on the presence or absence of the condition identified in the patient. And, for example, in other embodiments of the present application, the method further comprises determining whether to administer an additional dose or additional doses of the multimeric ligand to the patient based upon the appearance of graft versus host disease symptoms in the patient. In some embodiments, the method further comprises identifying the presence, absence or stage of graft versus host disease in the patient, and administering a multimeric ligand that binds to the multimerizing region, maintaining a subsequent dosage of the multimeric ligand, or adjusting a subsequent dosage of the multimeric ligand to the patient based on the presence, absence or stage of the graft versus host disease identified in the patient. In some embodiments, the method further comprises identifying the presence, absence or stage of graft versus host disease in the patient, and determining whether a multimeric ligand that binds to the multimerizing region should be administered to the patient, or the dosage of the multimeric ligand subsequently administered to the patient is adjusted based on the presence, absence or stage of the graft versus host disease identified in the patient. In some embodiments, the method further comprises receiving information comprising the presence, absence or stage of graft versus host disease in the patient; and administering a multimeric ligand that binds to the multimerizing region, maintaining a subsequent dosage of the multimeric ligand, or adjusting a subsequent dosage of the multimeric ligand to the patient based on the presence, absence or stage of the graft versus host disease identified in the patient. In some embodiments, the method further comprises identifying the presence, absence or stage of graft versus host disease in the patient, and transmitting the presence, absence or stage of the graft versus host disease to a decision maker who administers a multimeric ligand that binds to the multimerizing region, maintains a subsequent dosage of the multimeric ligand, or adjusts a subsequent dosage of the multimeric ligand administered to the patient based on the presence, absence or stage of the graft versus host disease identified in the subject. In some embodiments, the method further comprises identifying the presence, absence or stage of graft versus host disease in the patient, and transmitting an indication to administer a multimeric ligand that binds to the multimeric binding region, maintain a subsequent dosage of the multimeric ligand or adjust a subsequent dosage of the multimeric ligand administered to the patient based on the presence, absence or stage of the graft versus host disease identified in the subject.

Also provided is a method for administering donor T cells to a human patient, comprising administering a transduced or transfected T cell of the present application to a human patient, wherein the cells are non-allodepleted human donor T cells.

In some embodiments, the therapeutic cells are administered to a subject having a non-malignant disorder, or where the subject has been diagnosed with a non-malignant disorder, such as, for example, a primary immune deficiency disorder (for example, but not limited to, Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCK 8 Deficiency, IL-10 Deficiency/IL-10 Receptor Deficiency, GATA 2 deficiency, X-linked lymphoproliferative disease (XLP), Cartilage Hair Hypoplasia, and the like), Hemophagocytosis Lymphohistiocytosis (HLH) or other hemophagocytic disorders, Inherited Marrow Failure Disorders (such as, for example, but not limited to, Shwachman Diamond Syndrome, Diamond Blackfan Anemia, Dyskeratosis Congenita, Fanconi Anemia, Congenital Neutropenia, and the like), Hemoglobinopathies (such as, for example, but not limited to, Sickle Cell Disease, Thalassemia, and the like), Metabolic Disorders (such as, for example, but not limited to, Mucopolysaccharidosis, Sphingolipidoses, and the like), or an Osteoclast disorder (such as, for example, but not limited to Osteopetrosis).

The therapeutic cells may be, for example, any cell administered to a patient for a desired therapeutic result. The cells may be, for example, T cells, natural killer cells, B cells, macrophages, peripheral blood cells, hematopoietic progenitor cells, bone marrow cells, or tumor cells. The modified Caspase-9 polypeptide can also be used to directly kill tumor cells. In one application, vectors comprising polynucleotides coding for the inducible modified Caspase-9 polypeptide would be injected into a tumor and after 10-24 hours (to permit protein expression), the ligand inducer, such as, for example, AP1903, would be administered to trigger apoptosis, causing the release of tumor antigens to the microenvironment. To further improve the tumor microenvironment to be more immunogenic, the treatment may be combined with one or more adjuvants (e.g., IL-12, TLRs, IDO inhibitors, etc.). In some embodiments, the cells may be delivered to treat a solid tumor, such as, for example, delivery of the cells to a tumor bed. In some embodiments, a polynucleotide encoding the chimeric Caspase-9 polypeptide may be administered as part of a vaccine, or by direct delivery to a tumor bed, resulting in expression of the chimeric Caspase-9 polypeptide in the tumor cells, followed by apoptosis of tumor cells following administration of the ligand inducer. Thus, also provided in some embodiments are nucleic acid vaccines, such as DNA vaccines, wherein the vaccine comprises a nucleic acid comprising a polynucleotide that encodes an inducible, or modified inducible Caspase-9 polypeptide of the present application. The vaccine may be administered to a subject, thereby transforming or transducing target cells in vivo. The ligand inducer is then administered following the methods of the present application.

In some embodiments, the modified Caspase-9 polypeptide is a truncated modified Caspase-9 polypeptide. In some embodiments, the modified Caspase-9 polypeptide lacks the Caspase recruitment domain. In some embodiments, the Caspase-9 polypeptide comprises the amino acid sequence of SEQ ID NO: 9, or a fragment thereof, or is encoded by the nucleotide sequence of SEQ ID NO: 8, or a fragment thereof.

In some embodiments, the methods further comprise administering a multimeric ligand that binds to the multimeric ligand binding region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506-like analog ligand. In some embodiments, the multimeric ligand is AP1903. In some embodiments, the number of therapeutic cells is reduced by from about 60% to 99%, about 70% to 95%, from 80% to 90% or about 90% or more after administration of the multimeric ligand. In some embodiments, after administration of the multimeric ligand, donor T cells survive in the patient that are able to expand and are reactive to viruses and fungi. In some embodiments, after administration of the multimeric ligand, donor T cells survive in the patient that are able to expand and are reactive to tumor cells in the patient.

In some embodiments, the suicide gene used in the second level of control is a caspase polypeptide, for example, Caspase 1, 2, 3, 4, 5, 6, 7, 8, 9, 10, 11, 12, 13, or 14. In certain embodiments, the caspase polypeptide is a Caspase-9 polypeptide. In certain embodiments, the Caspase-9 polypeptide comprises an amino acid sequence of a catalytically active (not catalytically dead) caspase variant polypeptide provided in Table 5 or 6 herein. In other embodiments, the Caspase-9 polypeptide consists of an amino acid sequence of a catalytically active (not catalytically dead) caspase variant polypeptide provided in Table 5 or 6 herein. In other embodiments, a caspase polypeptide may be used that has a lower basal activity in the absence of the ligand inducer. For example, when included as part of a chimeric inducible caspase polypeptide, certain modified Caspase-9 polypeptides may have lower basal activity compared to wild type Caspase-9 in the chimeric construct. For example, the modified Caspase-9 polypeptide may comprise an amino acid sequence having at least 90% sequence identity to SEQ ID NO: 9, and may comprise at least one amino acid substitution.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A illustrates various iCasp9 expression vectors as discussed herein. FIG. 1B illustrates a representative western blot of full length and truncated Caspase-9 protein produced by the expression vectors shown in FIG. 1A. FIG. 1A discloses “GCCACC” as SEQ ID NO: 923 and “Ser-Gly-Gly-Gly-Ser” as SEQ ID NO: 924.

FIG. 2 is a schematic of the interaction of the suicide gene product and the CID to cause apoptosis.

FIG. 3 is a schematic depicting a two-tiered regulation of apoptosis. The left section depicts rapalog-mediated recruitment of an inducible caspase polypeptide to FRBI-modified CAR. The right section depicts a rimiducid (AP1903)-mediated inducible caspase polypeptide.

FIG. 4 is a plasmid map of a vector encoding FRB_{L}-modified CD19-MC-CAR and inducible Caspase-9. pSFG-iCasp9-2A-CD19-Q-CD28stm-MCz-FRB_{L}2.

FIG. 5 is a plasmid map of a vector encoding FRB_{L}-modified Her2-MC-CAR and an inducible Caspase-9 polypeptide. pSFG-iCasp9-2A-aHer2-Q_CD28stm-mMCz-FRB_{L}2.

FIGS. 6A and 6B provide the results of an assay of two-tiered activation of apoptosis. FIG. 6A shows recruitment of an inducible Caspase-9 polypeptide (iC9) with rapamycin, leading to more gradual apoptosis titration. FIG. 6B shows complete apoptosis using rimiducid (AP1903).

FIG. 7 is a plasmid map of the pBP0545 vector, pBP0545.pSFG.iCasp9.2A.Her2scFv.Q.CD8stm.MC-zeta.

FIGS. 8A-8C illustrate that FRB or FKBP12-based scaffolds can multimerize signaling domains. FIG. 8A. Homodimerization of a signaling domain (red stick), like Caspase-9, can be achieved via a heterodimer that binds to the FRB-fused signaling domain on one side and FKBP12-fused domain on the other. FIG. 8B. Dimerization or multimerization of a signaling domain via 2 (left) or more (right) tandem copies of FRB (chevron). The scaffold can contain subcellular targeting sequences to localize proteins to the plasma membrane (as depicted), the nucleus or organelles. FIG. 8C. Similar to FIG. 8B, but domain polarity is reversed.

FIGS. 9A-9C provide schematics of iMC-mediated scaffolding of FRB_{L}2. Caspase-9. FIG. 9A. In the presence of a heterodimer drug, such as a rapamycin, the FRB_{L}2-linked Caspase-9 binds with and clusters the FKBP-modified MyD88/CD40 (MC) signaling molecule. This clustering effect results in dimerization of FRB_{L}2. Caspase-9 and subsequent induction of cellular death via the apoptotic pathway. FIG. 9B. Similar to panel 9A, however the FKBP and FRB domains have been switched in relation to associated Caspase-9 and MC domains. The clustering effect still occurs in the presence of heterodimer drug. FIG. 9C. Similar to panel 9A; however there is only one FKBP domain attached to MC. Therefore, in the presence of heterodimer, Caspase-9 is no longer capable of being clustered and therefore apoptosis is not induced.

FIG. 10A-10E provide schematics of a rapalog-induced, FRB scaffold-based inducible Caspase-9 polypeptide. FIG. 10A: Rimiducid homodimerizes FKBPv-linked Caspase-9, resulting in dimerization and activation of Caspase-9 with subsequent induction of cellular death via the apoptotic pathway. FIG. 10B: Rapalogs heterodimerize FKBPv-linked Caspase-9 with FRB-linked Caspase-9, resulting in dimerization of Caspase-9 and cell death. FIG. 10C, FIG. 10D, FIG. 10E are schematics illustrating that in the presence of a heterodimer drug, such as a rapalog, 2 or more FRB_{L} domains act as a scaffold to recruit binding of FKBPv-linked Caspase-9, leading to dimerization or oligomerization of Caspase-9 and cell death.

FIG. 11A is a schematic and FIG. 11B is a line graph depicting activation of apoptosis by dimerization of a chimeric FRB-Caspase-9 polypeptide and a chimeric FKBP-Caspase-9 polypeptide (FRB_{L}-ΔCaspase-9 and FKBPv-ΔCaspase-9) with rapamycin. FIG. 11A. Schematic representation of dimerization of FRB and FKBP12 with rapamycin to bring together fused Caspase-9 signaling domains and activation of apoptosis. FIG. 11B. Reporter assays were performed in HEK-293T cells transfected with the constitutive SRα-SEAP reporter (pBP046, 1 μg), a fusion of FRB_{L} (L2098) and human ΔCaspase-9 (pBP0463, 2 μg) and a fusion of FKBP12 with ΔCaspase-9 (pBP0044, 2 μg).

FIG. 12A is a schematic and FIGS. 12B and 12C are line graphs depicting assembly of FKBP-Caspase-9 on a FRB-based scaffold. FIG. 12A: Schematic of iterated FRB domains to provide scaffolds for rapamycin (or rapalog)-mediated multimerization of an FKBP12-Caspase-9 fusion protein. FIG. 12B: Cultures of HEK-293 cells were transfected (via Genejuice, Novagen) with the constitutive SRα-SEAP reporter plasmid (pBP0046, 1 μg), a fusion of human FKBP12 with human Caspase-9 (pBP0044, 2 μg) and FRB-encoding expression constructs, containing four copies of FRB_{L} (pBP0725, 2 μg) or control vectors encoding zero or one copy of FRB_{L}. 24 hours post-transfection, cells were distributed into 96-well plates and rapamycin or a derivative rapalog, C7-isopropoxyrapamycin, with specificity for the mutant FRB_{L} (Liberles et al, 1997) were administered in triplicate wells. Placental SEAP reporter activity was determined 24 hours post-drug administration. FIG. 12C: Reporter assays were performed as in (B), but FRB-scaffolds were expressed from constructs encoding iterated FRB_{L} domains with an amino-terminal myristoylation-targeting sequence and two (pBP0465) or four copies (pBP0721) of the FRB_{L} domain.

FIG. 13A is a schematic and FIG. 13B is a line graph depicting assembly of FRB-ΔCaspase-9 on an FKBP scaffold. FIG. 13A. Schematic of iterated FKBP12 domains to produce scaffolds for assembly of rapamycin (or rapalog)-mediated multimerization of FRB-ΔCaspase-9 fusion protein, leading to apoptosis. FIG. 13B. Reporter assays were performed as in FIGS. 12B and C with cultures of HEK-293T cells transfected with the constitutive SRα-SEAP reporter (pBP046, 1 μg), a fusion of FRB_{L} (L2098) and CARD domain-deleted human ΔCaspase-9 (pBP0463, 2 μg) and FKBP expression constructs containing four tandem copies of FKBP12 (pBP722, 2 μg) or a control vector with one copy of FKBP (pS-SF1E).

FIGS. 14A-14B provide line graphs showing that heterodimerization of FRB_{L} scaffold with iCaspase9 induces cell death. Primary T cells from three different donors (307, 582, 584) were transduced with pBP0220-pSFG-iC9.T2A-ΔCD19, pBP0756-pSFG-iC9.T2A-ΔCD19.P2A-FRB_{L}, pBP0755-pSFG-iC9.T2A-ΔCD19.P2A-FRB_{L}2, or pBP0757-pSFG-iC9.T2A-ΔCD19.P2A-FRB_{L}3, containing iC9, CD19 marker, and 0-3 tandem copies of FRB_{L}, respectively. T Cells were plated with varying concentrations of rapamycin and after 24 and 48 hours cell aliquots were harvested, stained with APC-CD19 antibody and analyzed by flow cytometry. Cells were initially gated on live lymphocytes by FSC vs SSC. Lymphocytes were then plotted as a CD19 histogram and subgated for high, medium and low expression within the CD19⁺ gate. Line graphs represent the relative percentage of the total cell population that express high levels of CD19, normalized to the no “0” drug control. All data points were done in duplicates. FIG. 14A: donor 307, 24 hr; FIG. 14B: donor 582, 24 hr; FIG. 14C: donor 584 24 hr; FIG. 14D: donor 582 48 hr; FIG. 14E: donor 584 48 hr.

FIGS. 15A-15C provide line graphs and a schematic showing that rapamycin induces iC9 killing in the presence of tandem FRB_{L} domains. HEK-293 cells were transfected with 1 μg of SRα-SEAP constitutive reporter plasmid along with either negative (Neg) control, eGFP (pBP0047), iC9 (iC9/pBP0044) alone, or iC9 along with iMC.FRB_{L} (pBP0655)+anti-HER2.CAR.Fpk2 (pBP0488) or iMC.FRB_{L}2 (pBP0498)+anti-HER2.CAR.Fpk2. Cells were then plated with half-log dilutions of rimiducid or rapamycin and assayed for SEAP as previously described. Diminution of SEAP activity correlates with cell elimination. Schematic represents one possible rapamycin-mediated complex of signaling domains, which lead to Caspase-9 clustering and apoptosis. FIG. 15A: rimiducid; FIG. 15B: rapamycin; FIG. 15C: schematic.

FIGS. 16A and 16B are line graphs showing that tandem FKBP scaffold mediates FRB_{L}2. Caspase activation in the presence of rapalogs. FIG. 16A. HEK-293 cells were transfected with 1 μg each of SRα-SEAP reporter plasmid, Δmyr.iMC.2A-anti-CD19.CAR.CD3ζ (pBP0608), and FRB_{L}2. Caspase-9 (pBP0467). After 24 hours, transfected cells were harvested and treated with varying concentrations of either rimiducid, rapamycin, or rapalog, C7-isopropoxy (IsoP)-rapamycin. After ON incubation, cell supernatants were assayed for SEAP activity, as previously described. FIG. 16B. Similar to the experiment described in (FIG. 16A), except that cells were transfected with a membrane-localized (myristoylated) iMC.2A-CD19.CAR.CD3ζ (pBP0609), instead of non-myristoylated Δmyr.iMC.2A-CD19.CAR.CD3ζ (pBP0608).

FIGS. 17A-17E provides line graphs and the results of FACs analysis showing that the iMC “switch”, FKBP2.MyD88.CD40, creates a scaffold for FRB_{L}2. Caspase9 in the presence of rapamycin, inducing cell death. FIG. 17A. Primary T cells (2 donors) were transduced with γ-RV, SFG-ΔMyr.iMC.2A-CD19 (from pBP0606) and SFG-FRB_{L}2. Caspase9.2A-Q.8stm.zeta (from pBP0668). Cells were plated with 5-fold dilutions of rapamycin. After 24 hours, cells were harvested and analyzed by flow cytometry for expression of iMC (anti-CD19-APC), Caspase-9 (anti-CD34-PE), and T cell identity (anti-CD3-PerCPCy5.5). Cells were initially gated for lymphocyte morphology by FSC vs SSC, followed by CD3 expression (˜99% of the lymphocytes). CD3⁺ lymphocytes were plotted for CD19 (Δmyr.iMC.2A-CD19) vs CD34 (FRB_{L}2. Caspase9.2A-Q.8stm.zeta) expression.

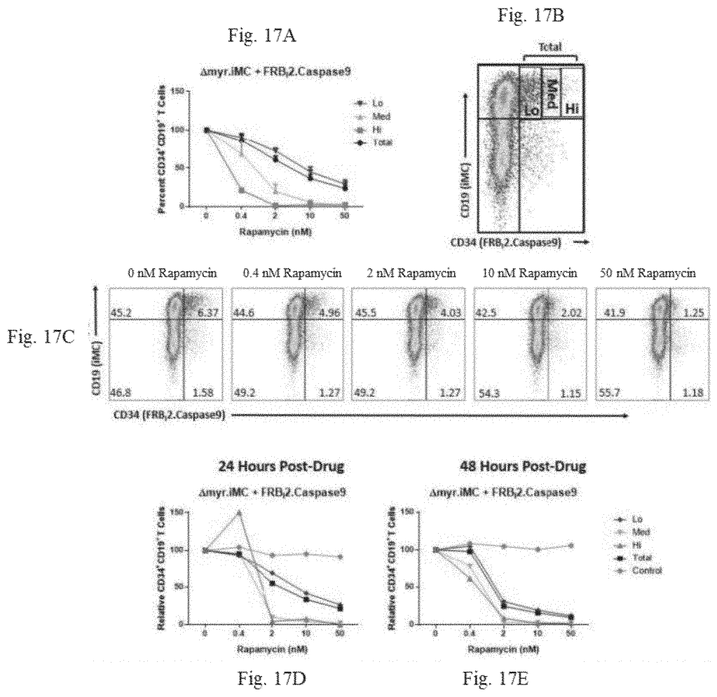

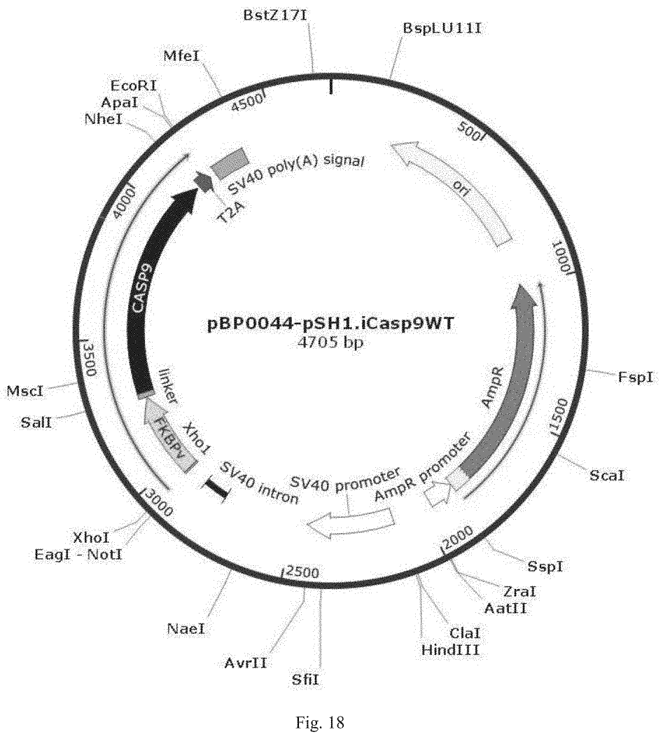

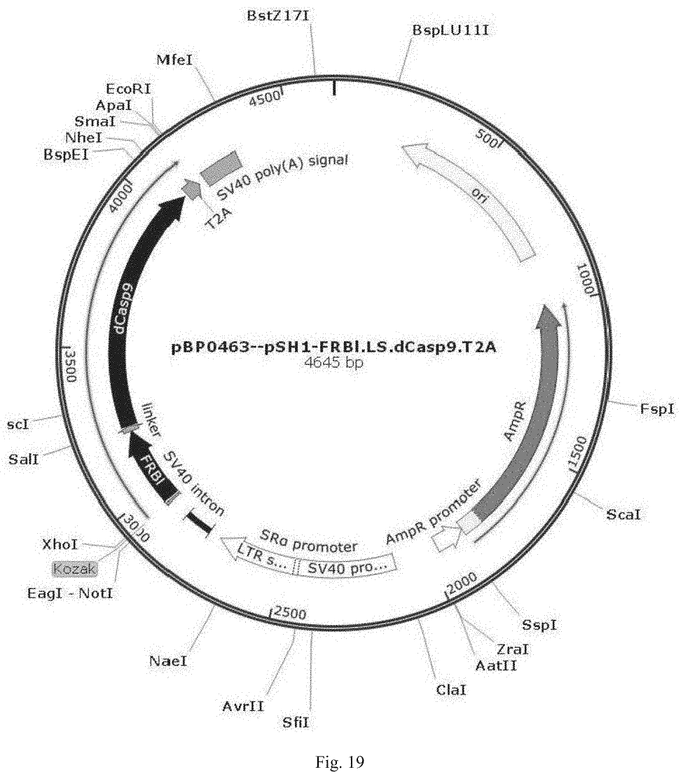

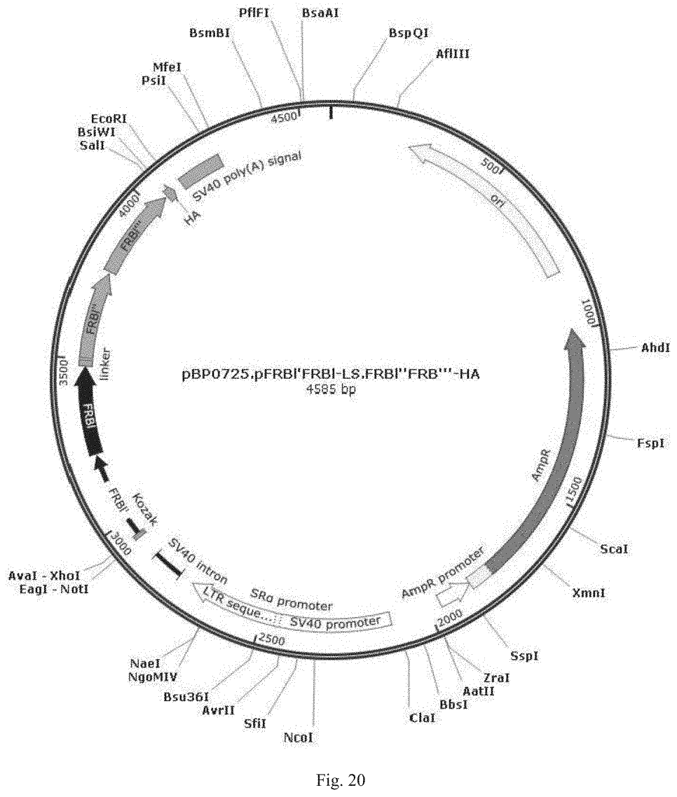

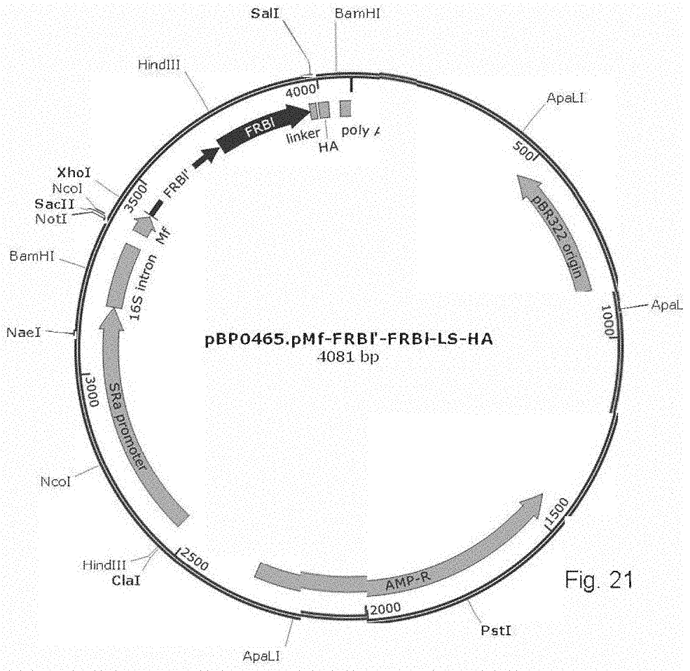

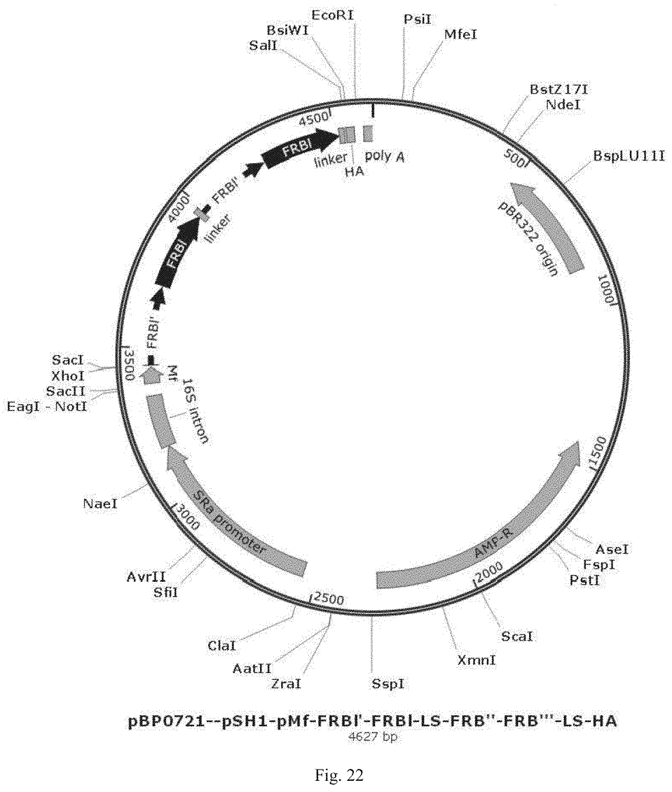

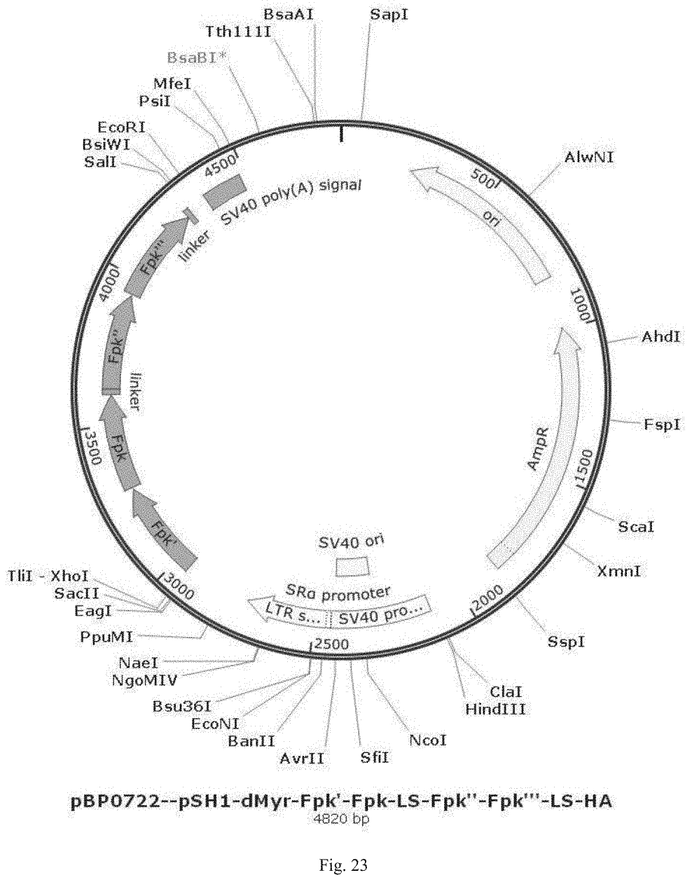

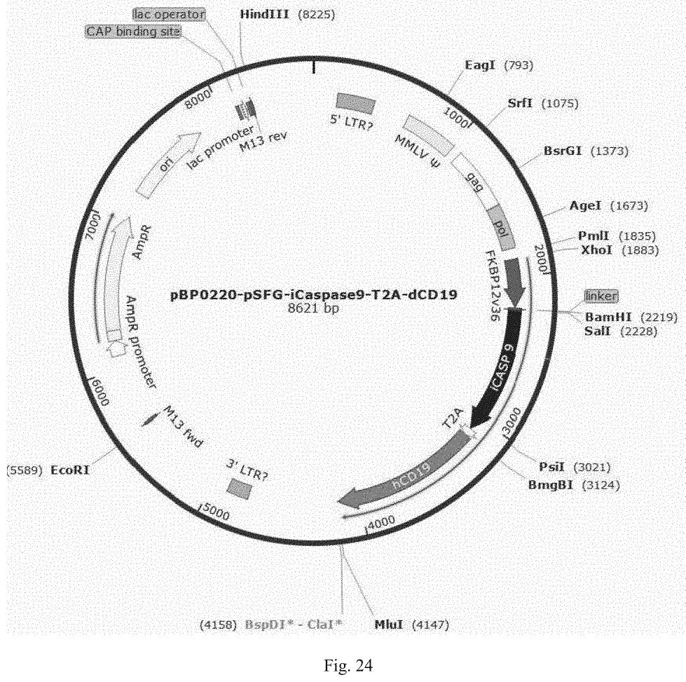

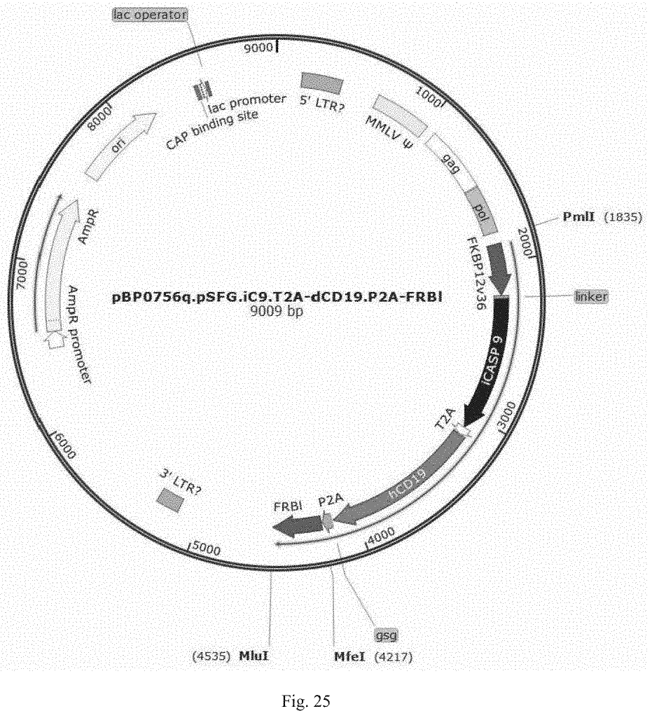

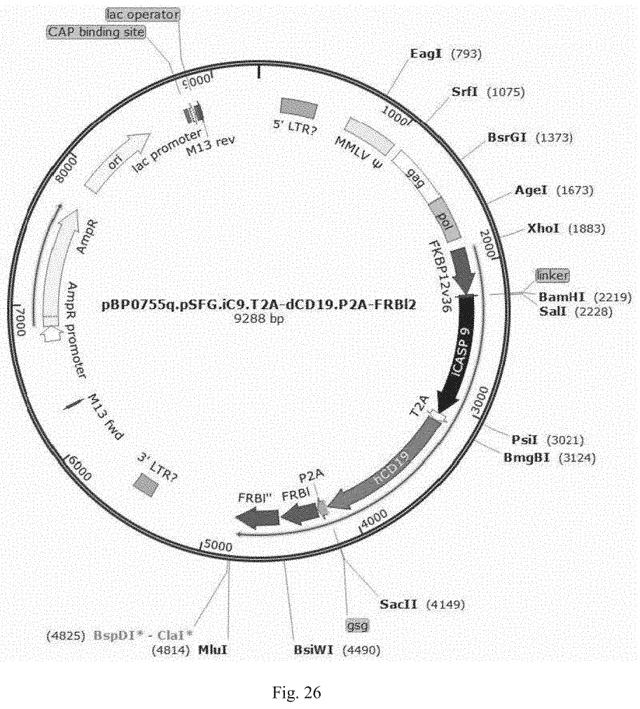

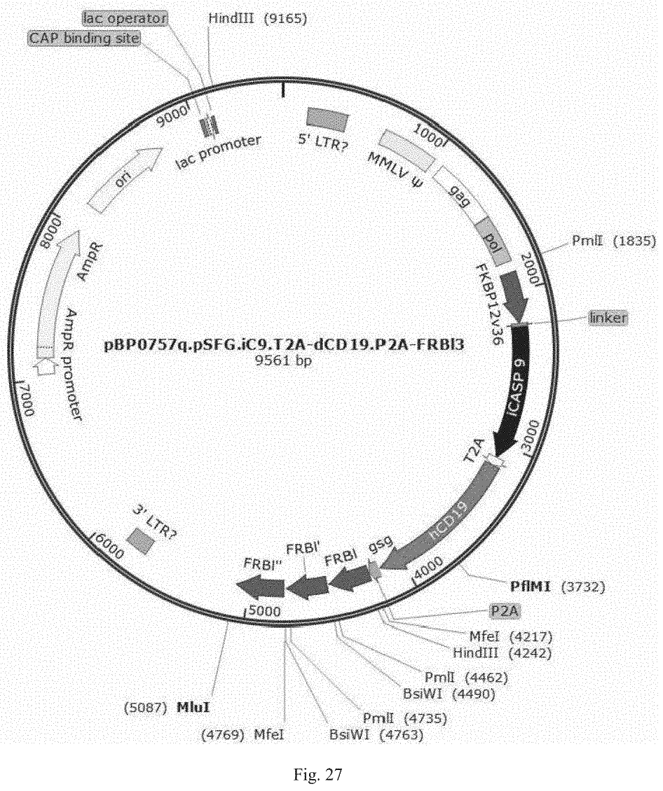

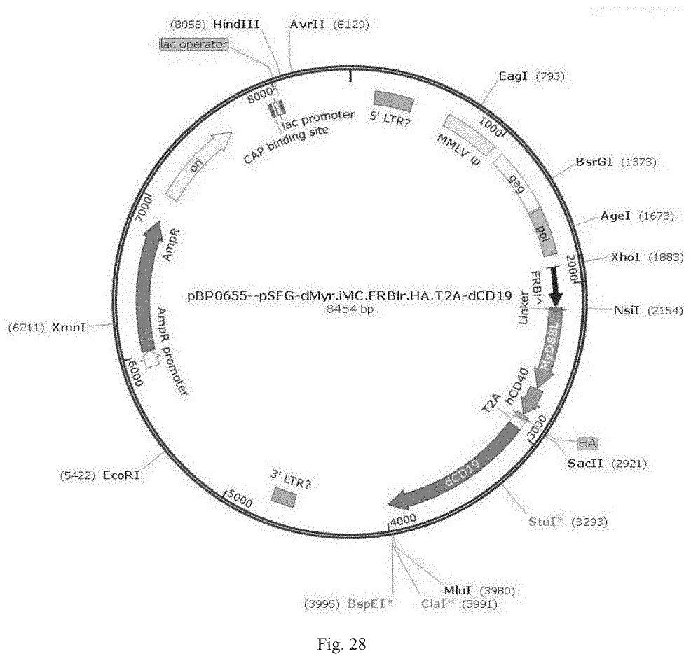

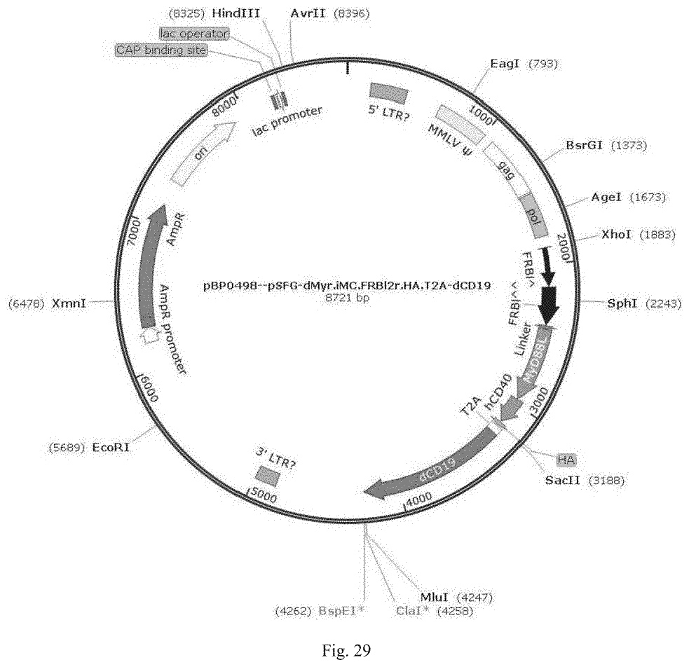

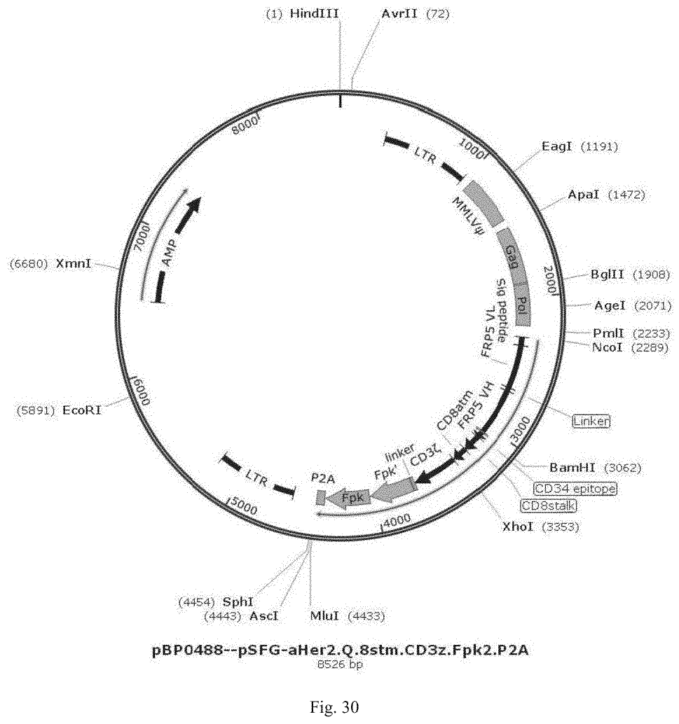

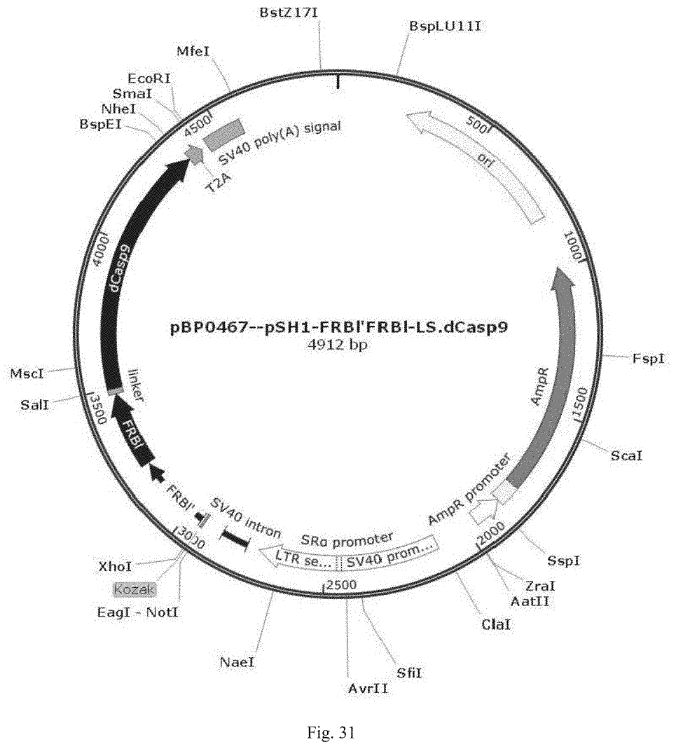

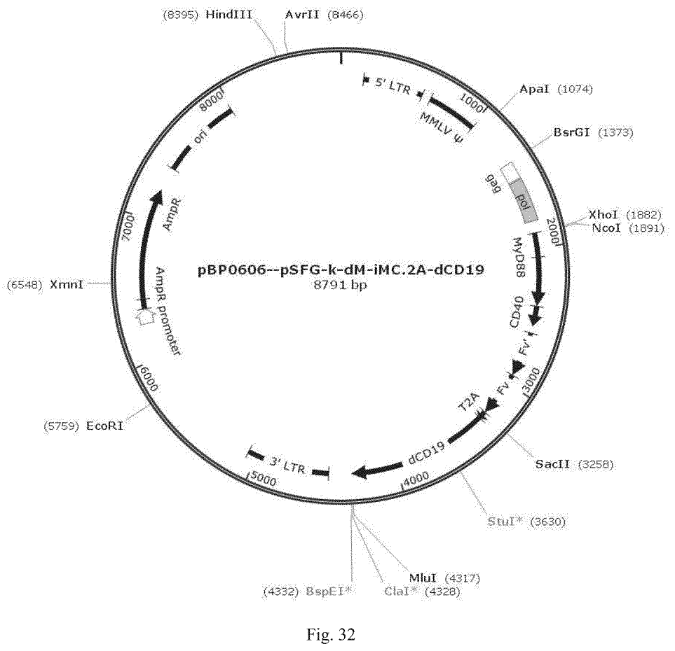

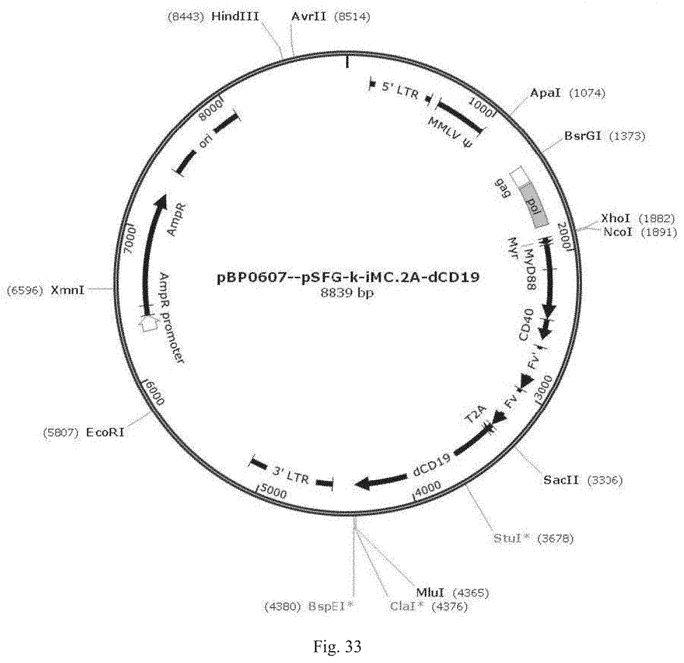

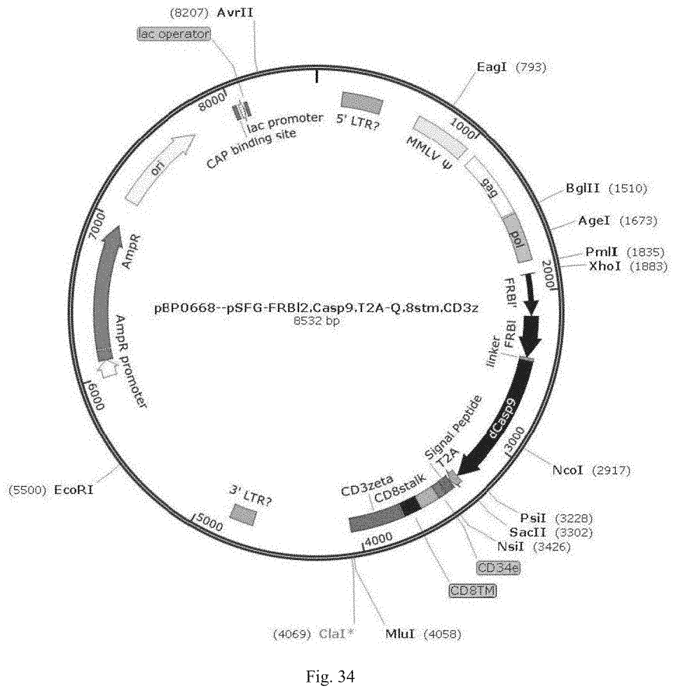

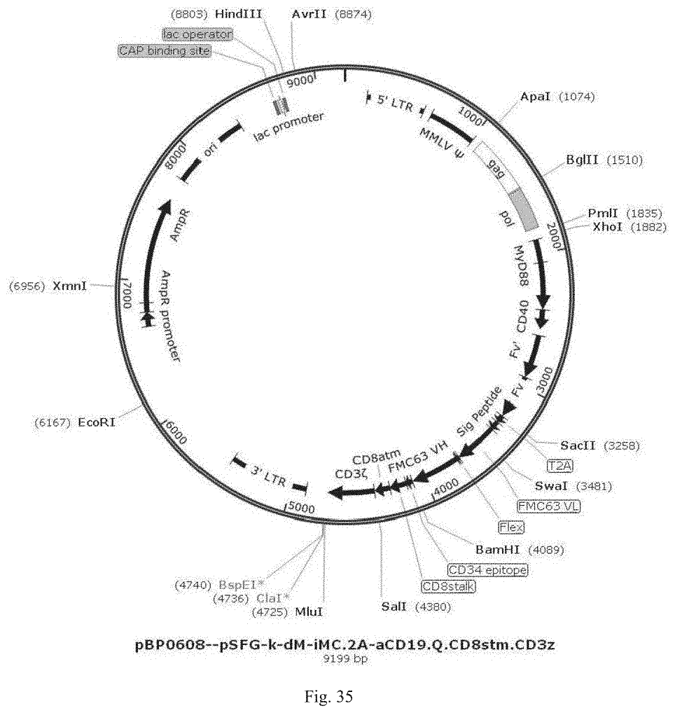

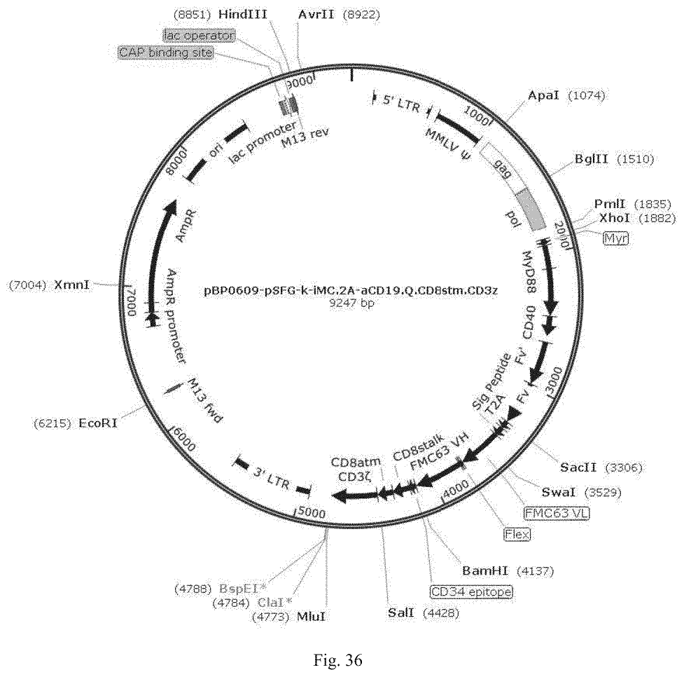

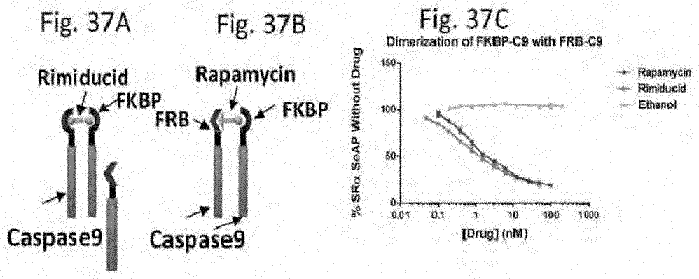

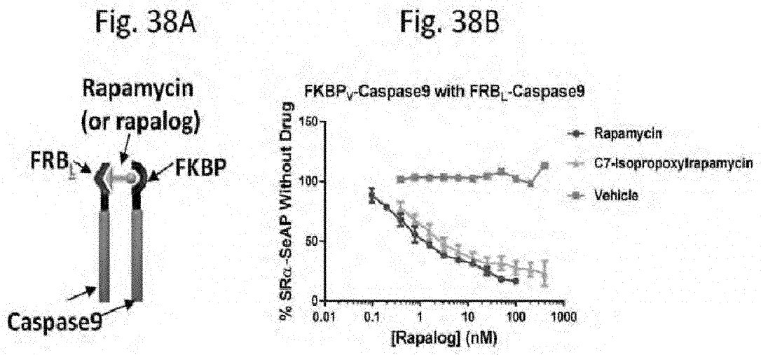

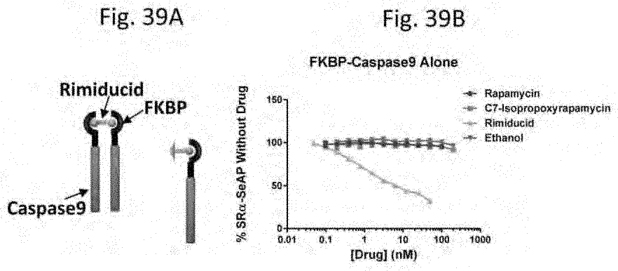

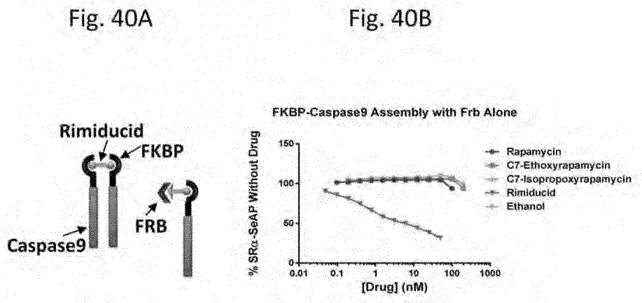

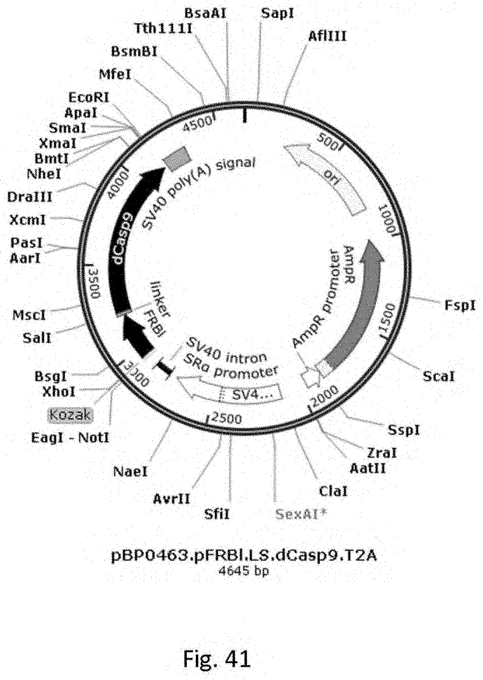

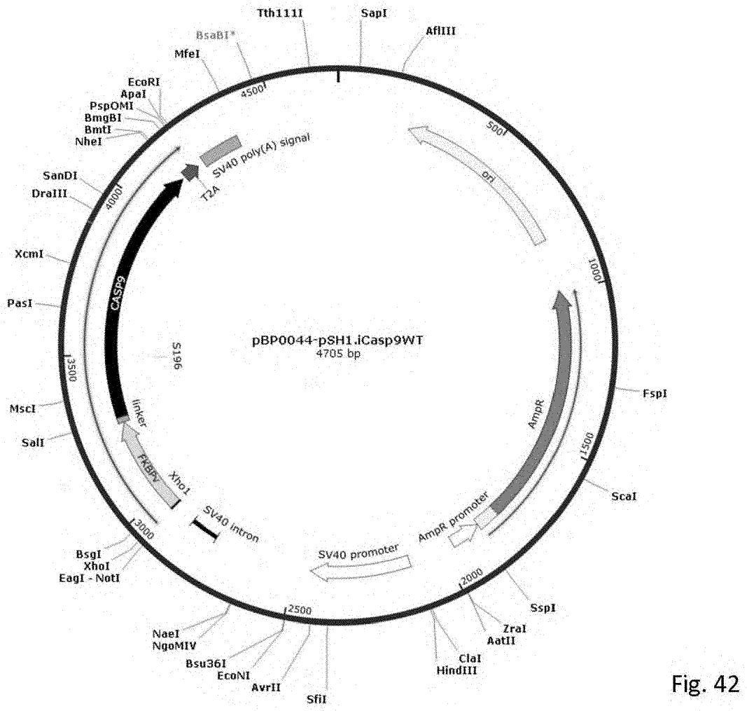

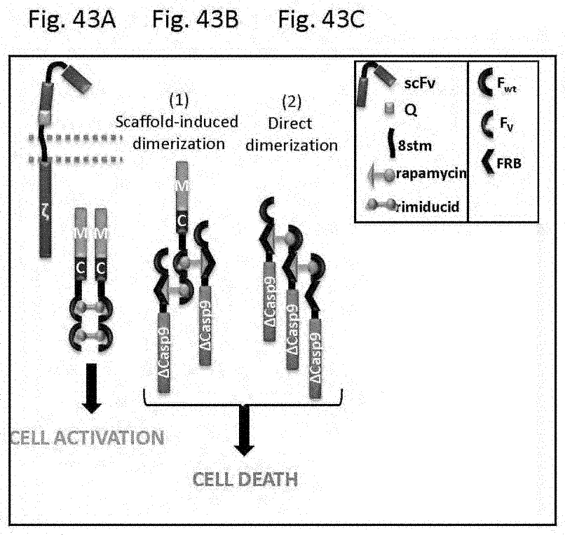

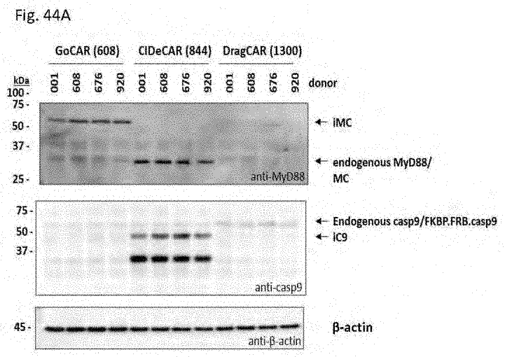

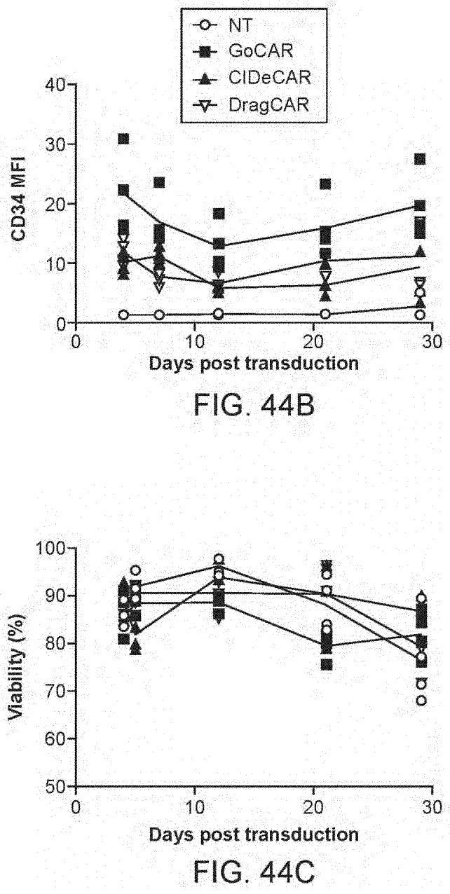

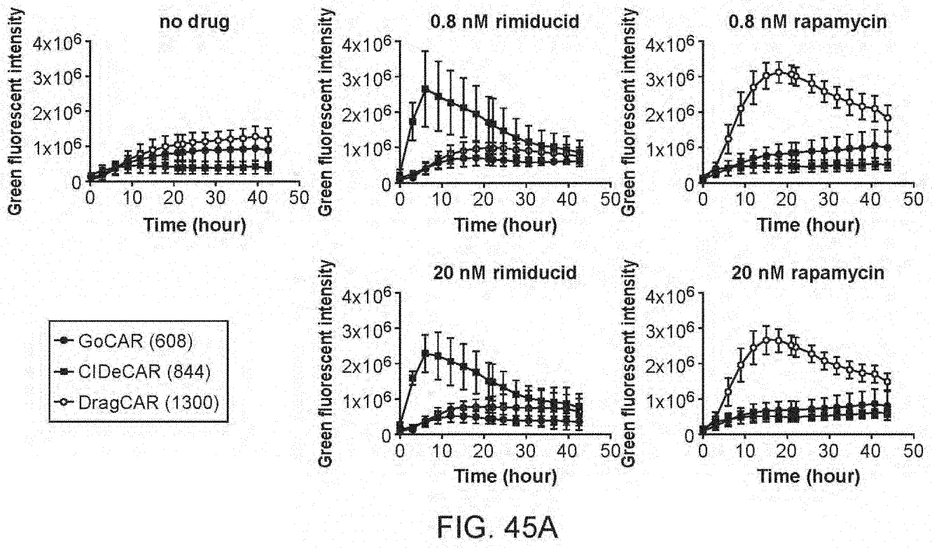

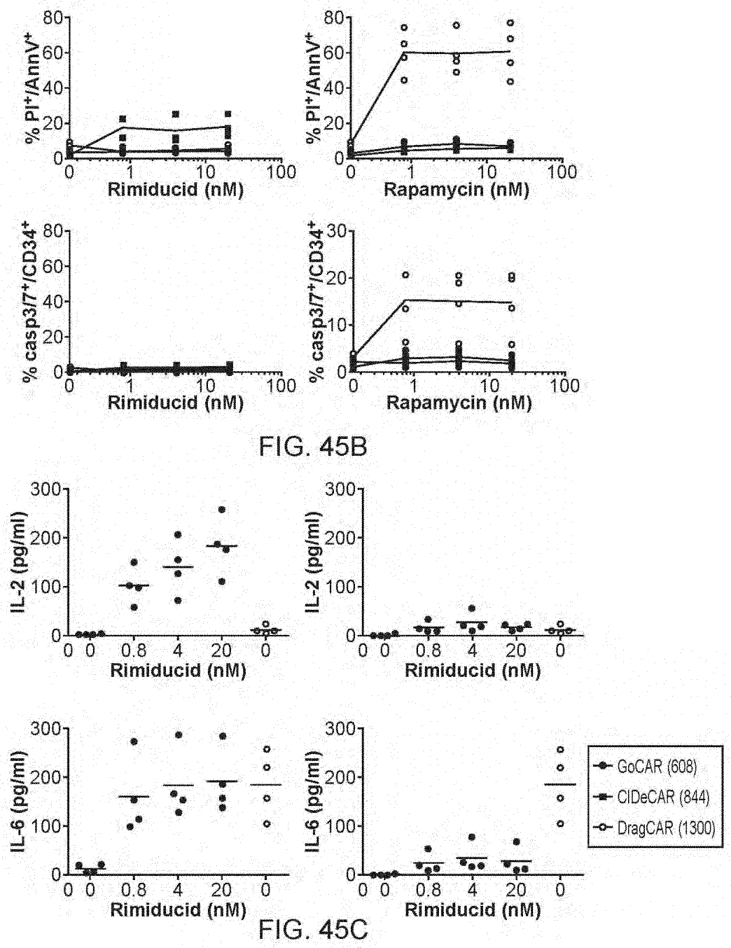

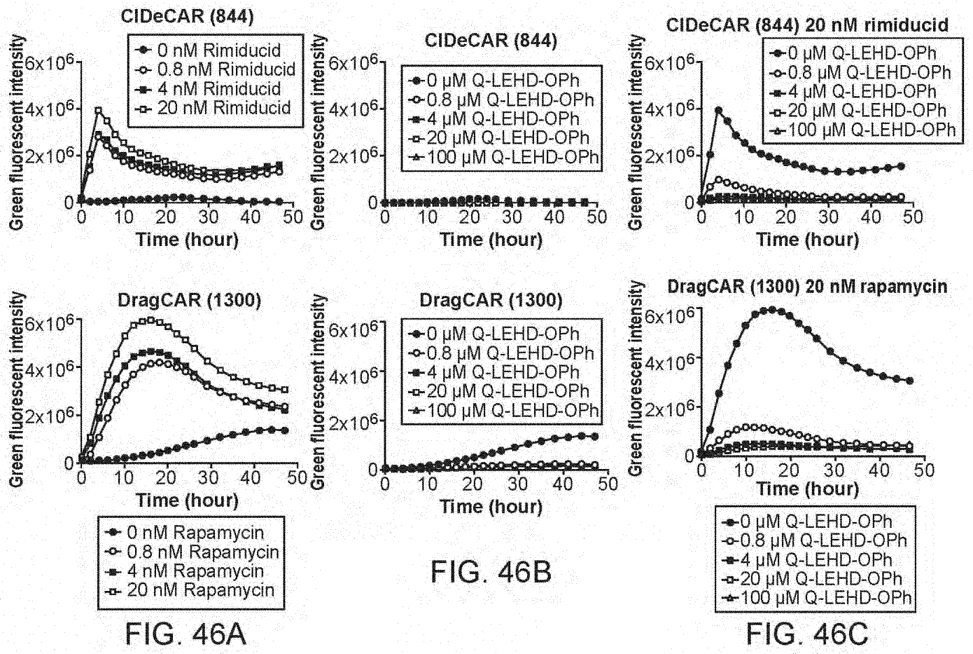

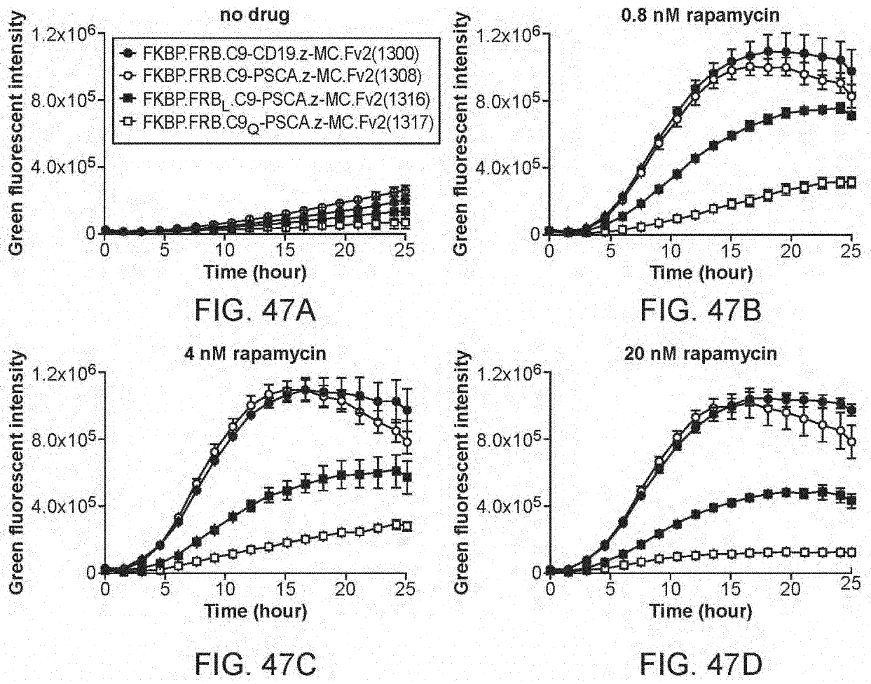

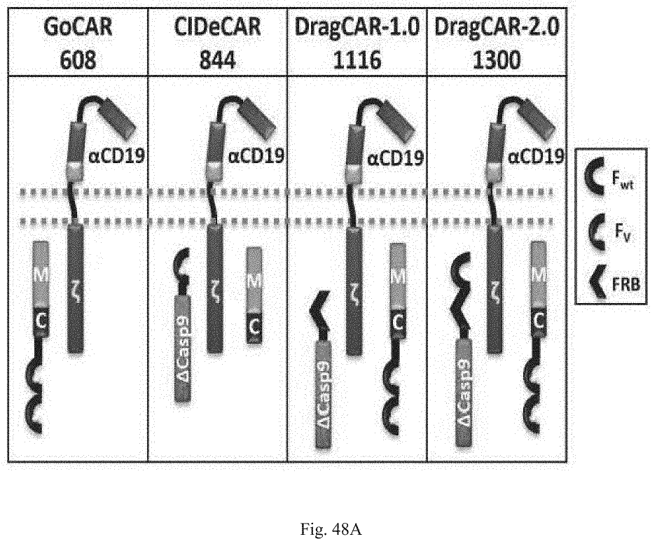

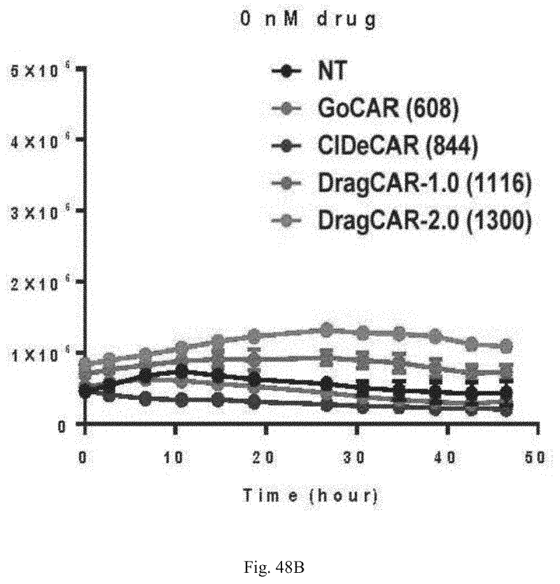

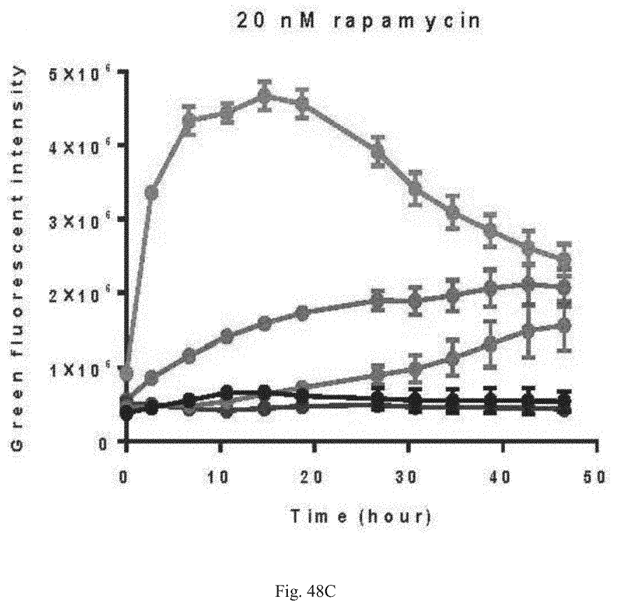

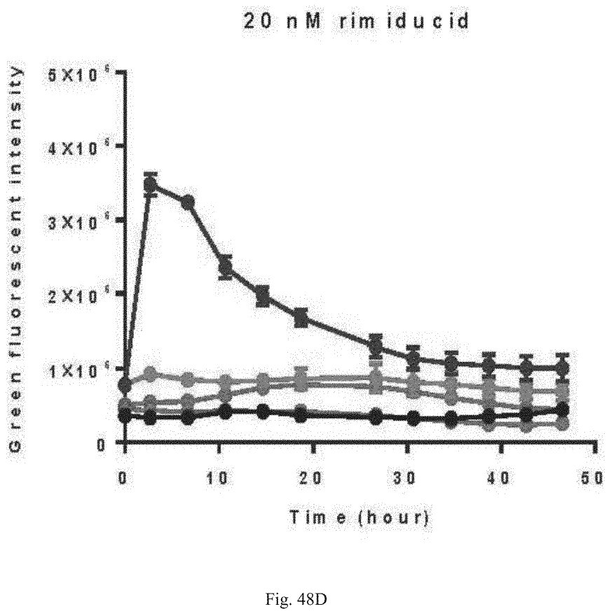

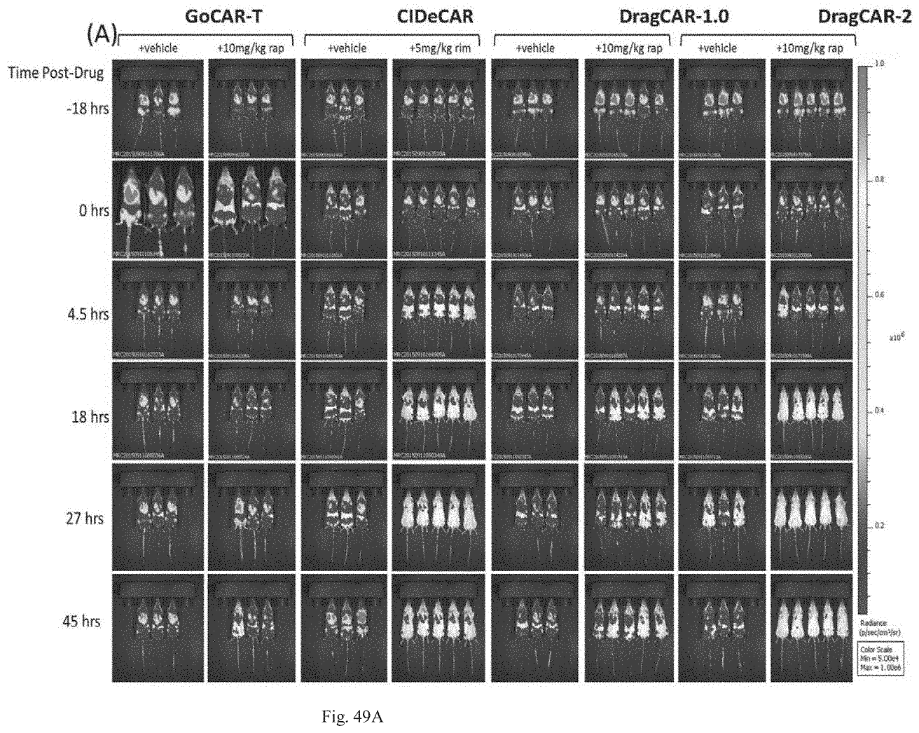

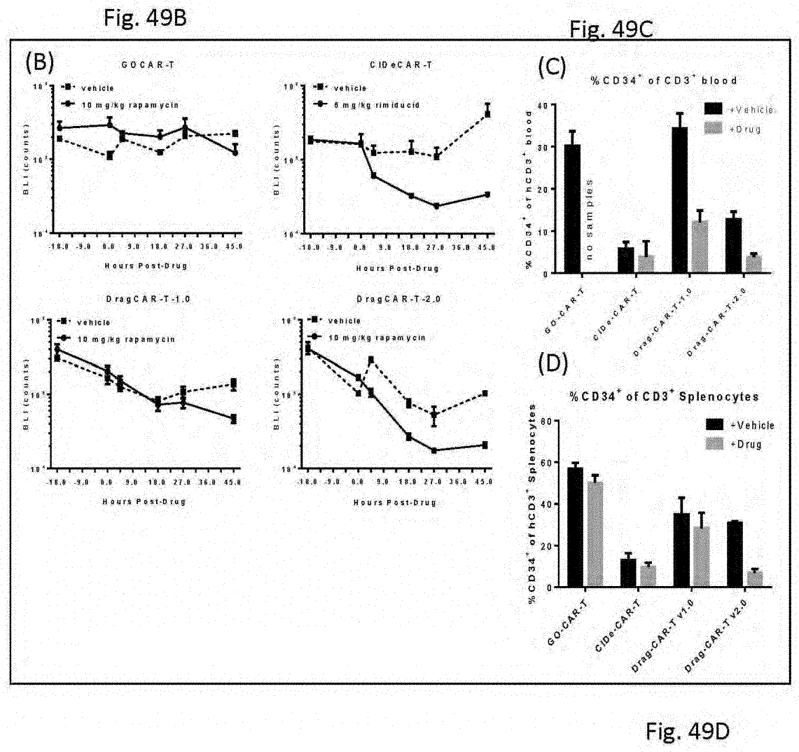

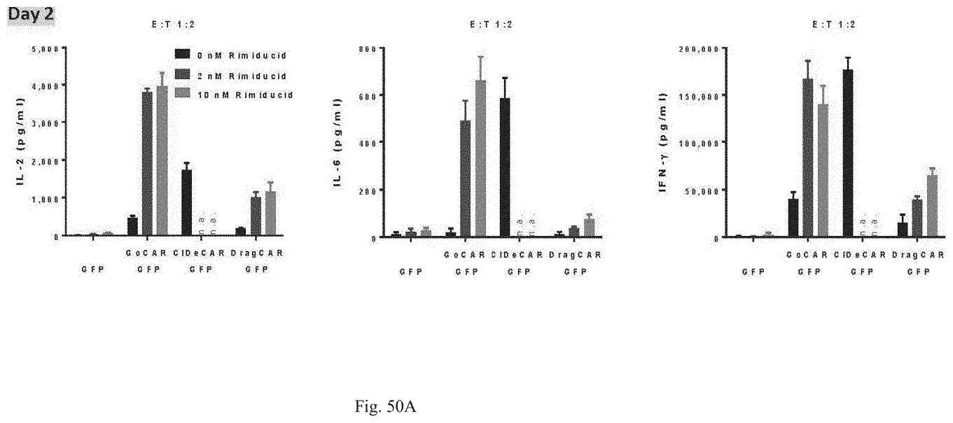

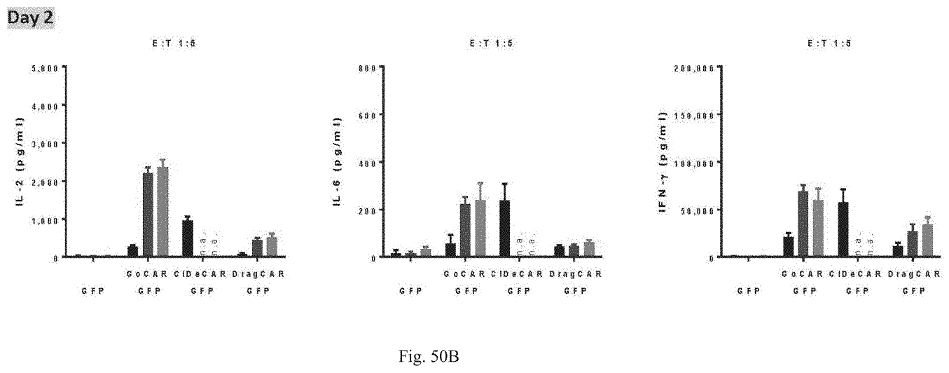

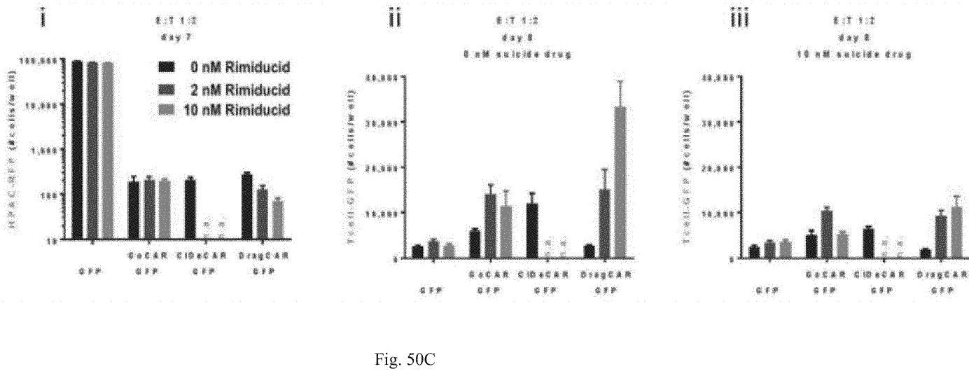

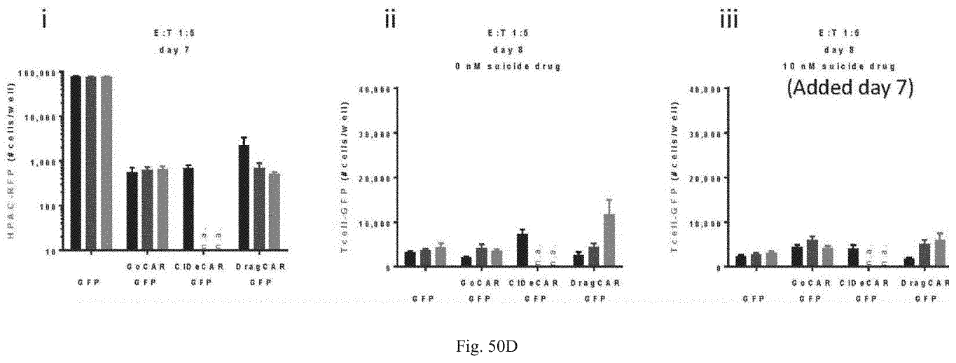

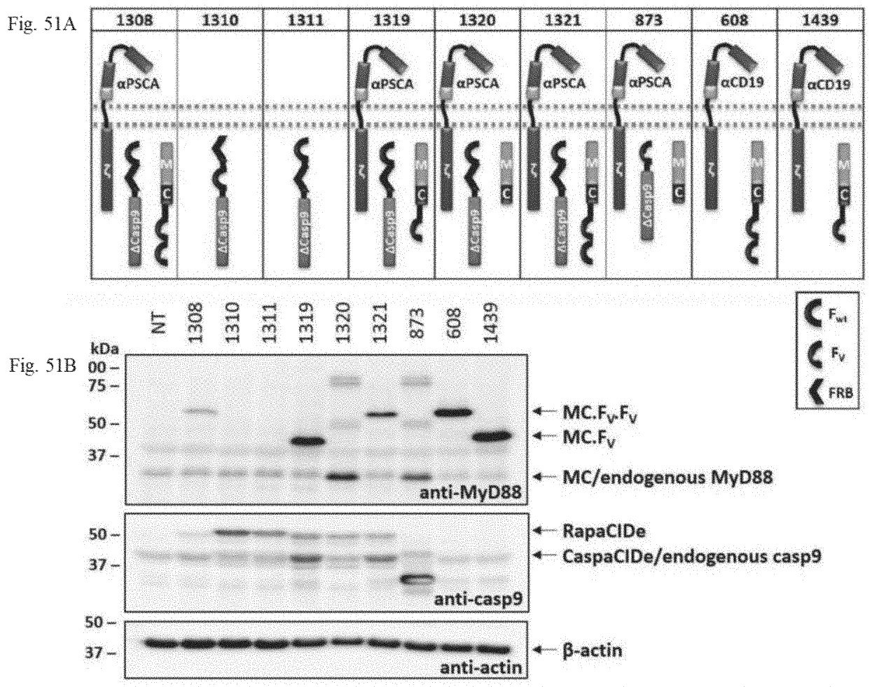

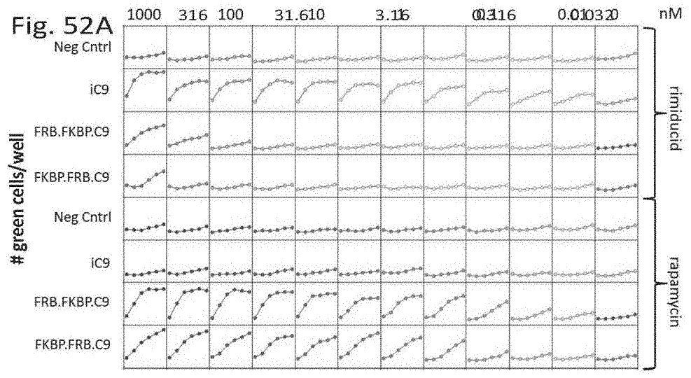

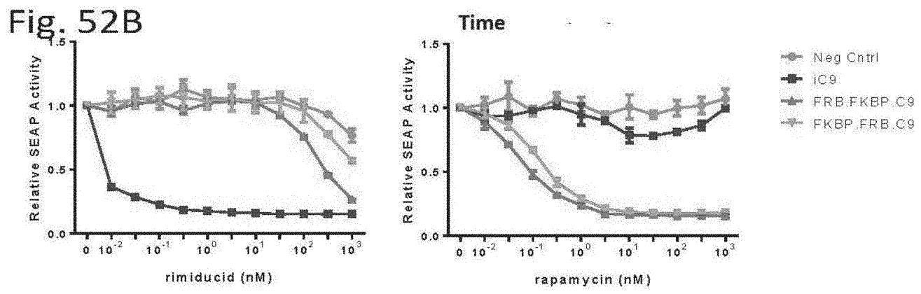

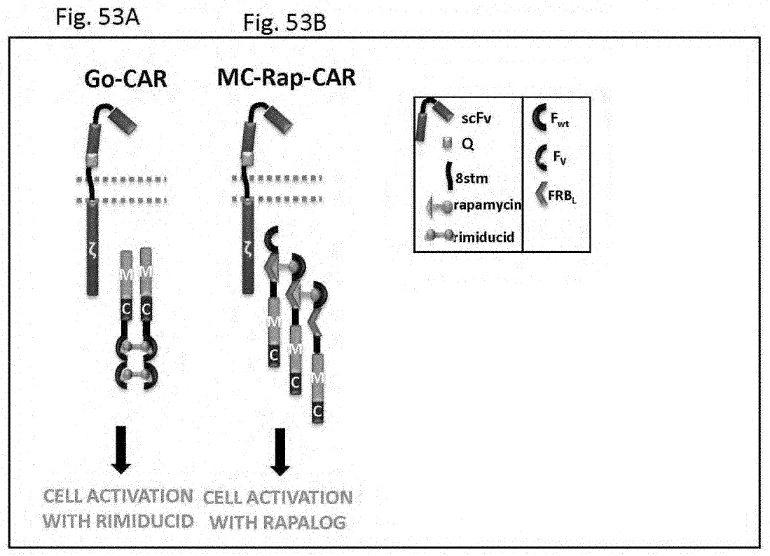

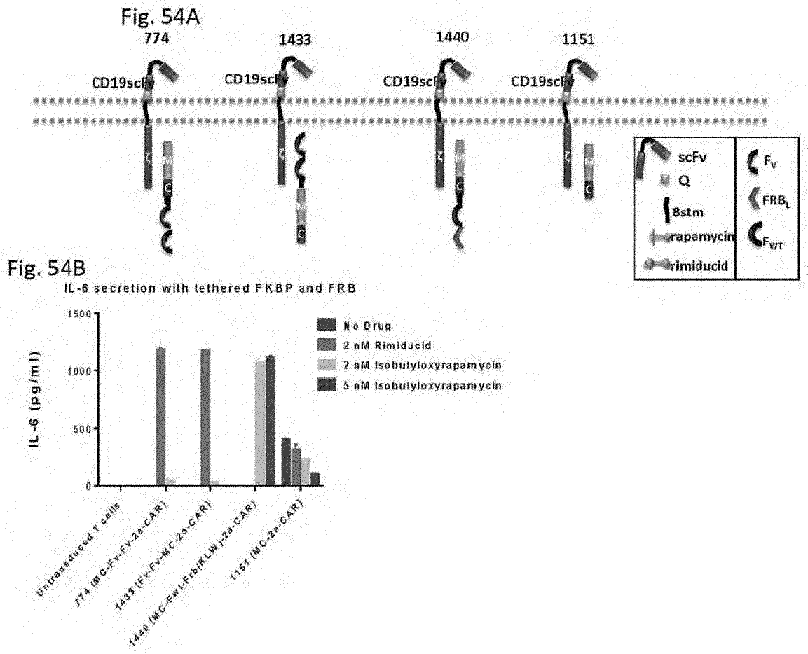

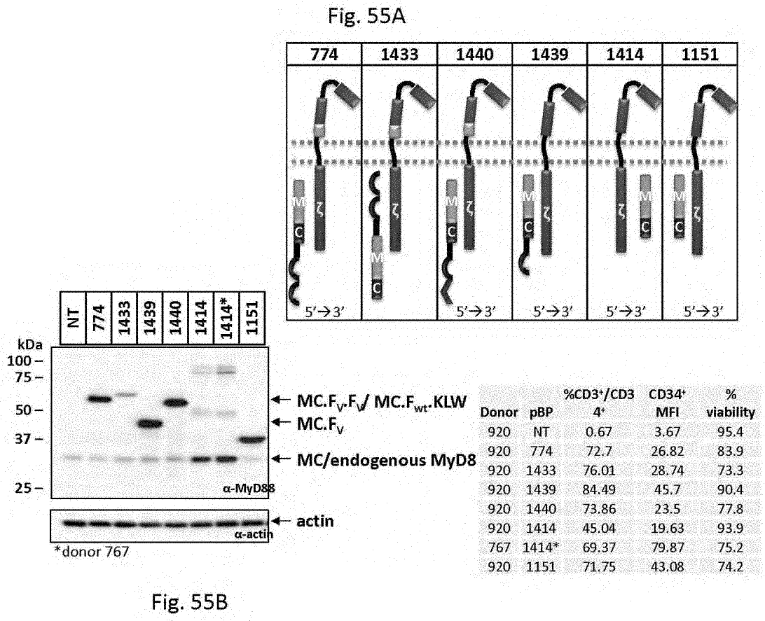

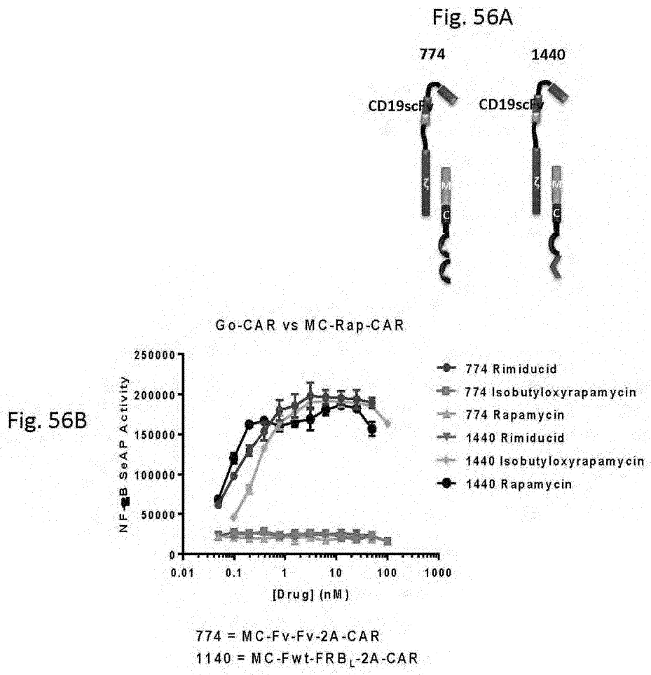

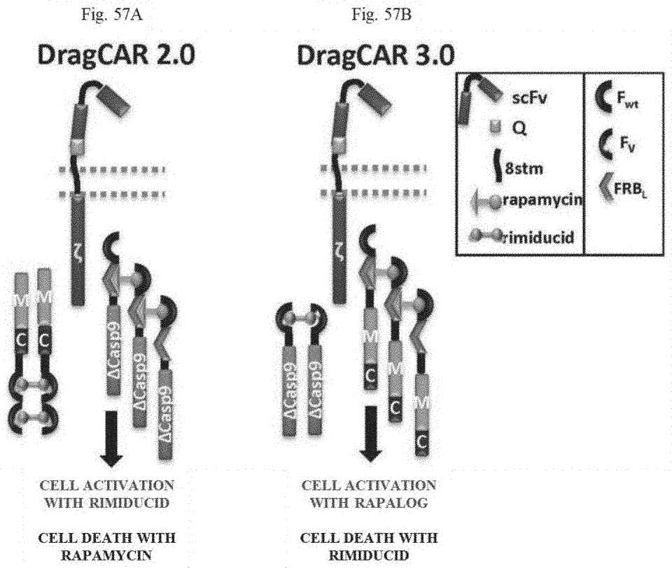

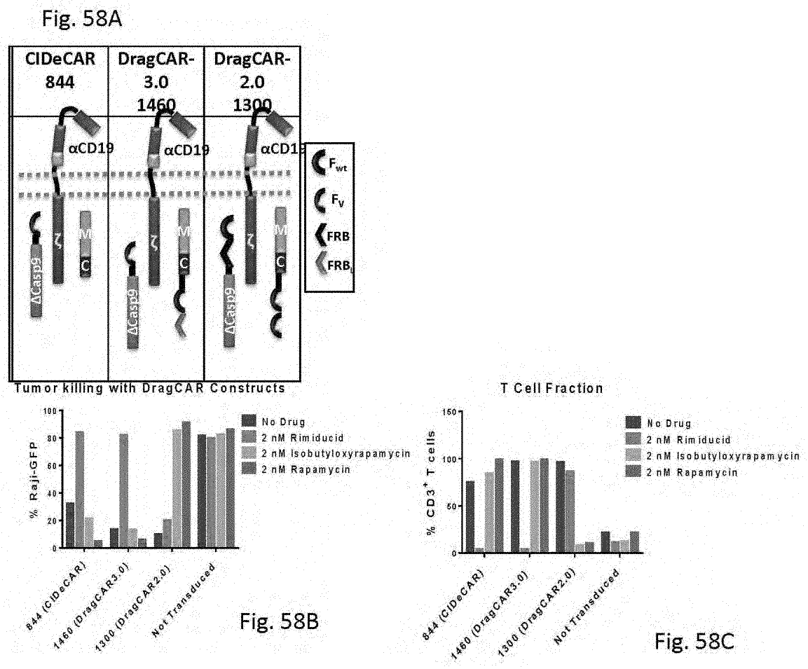

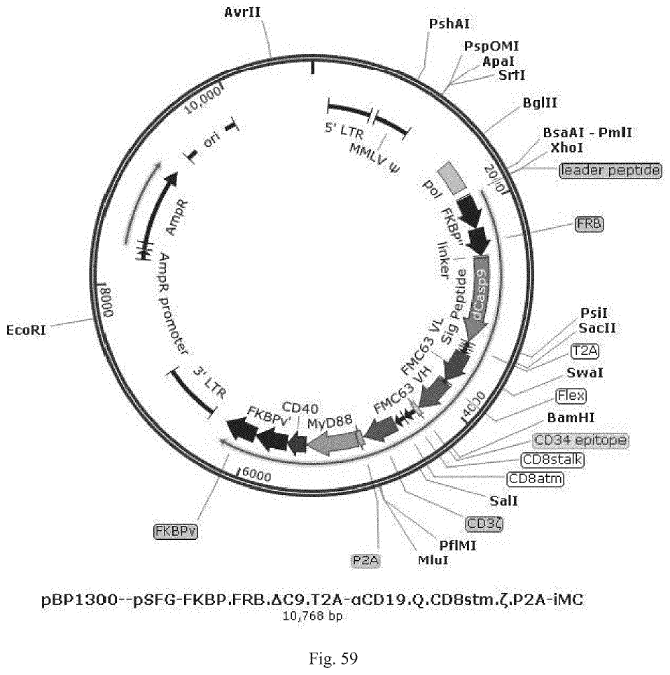

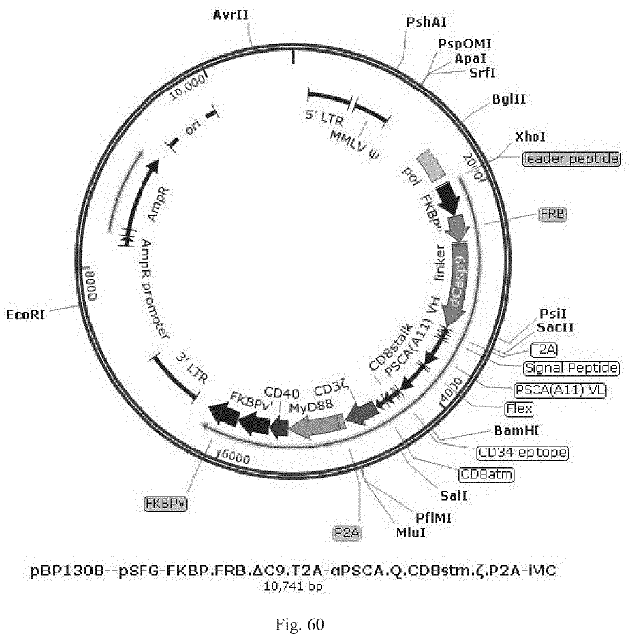

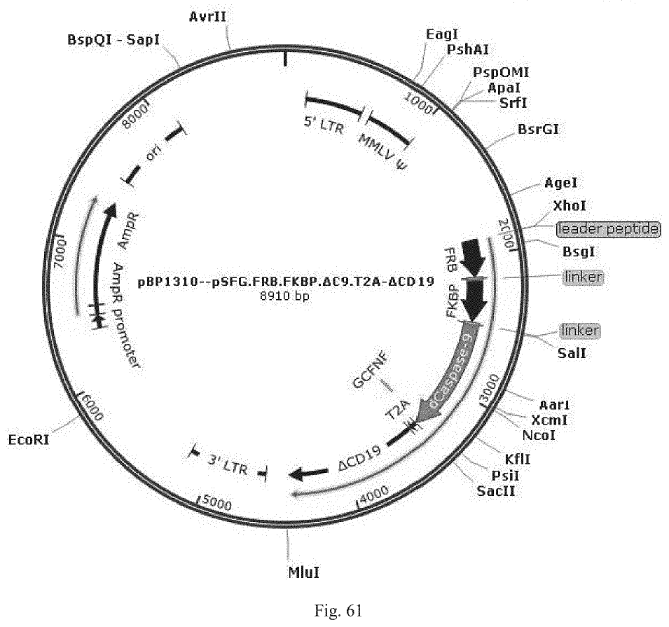

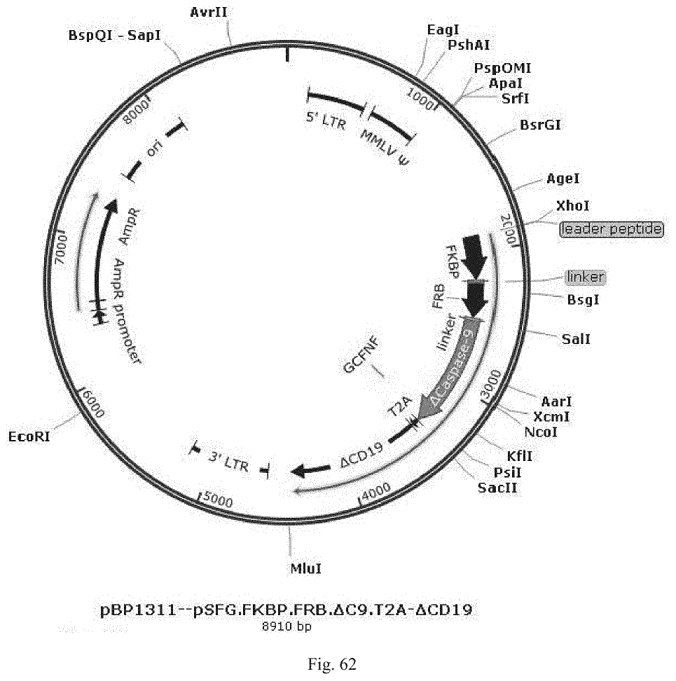

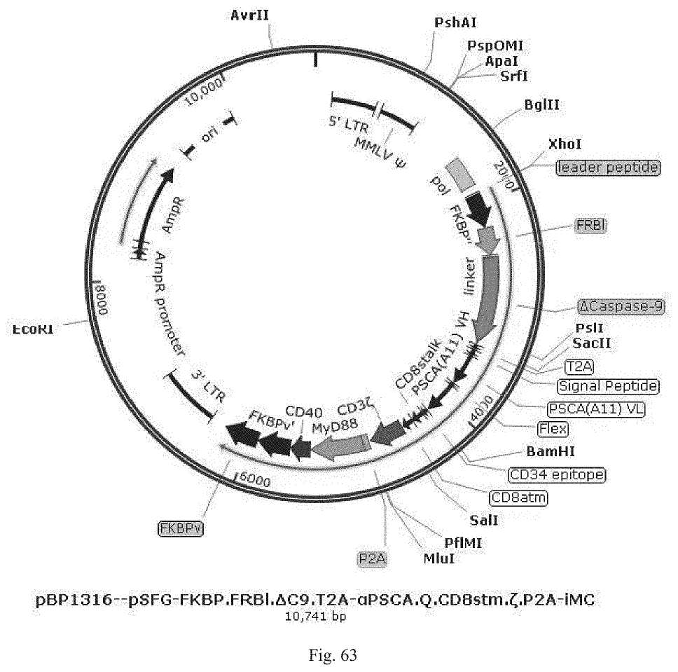

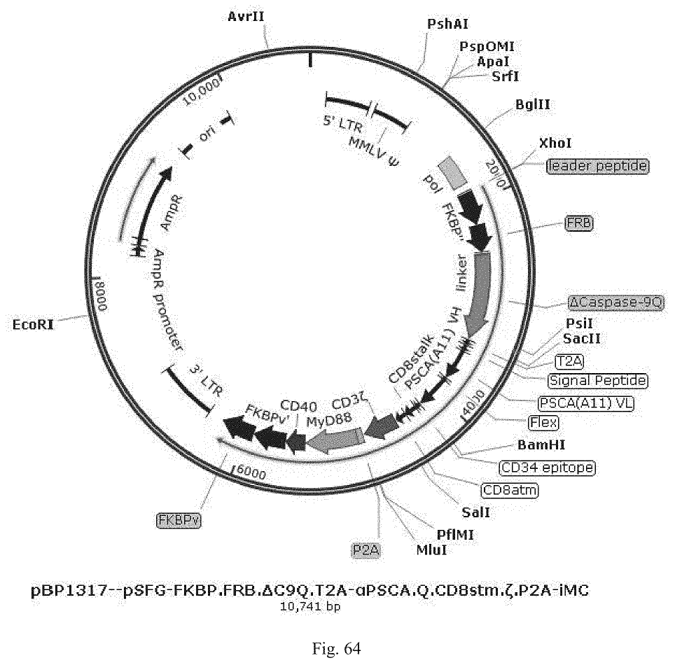

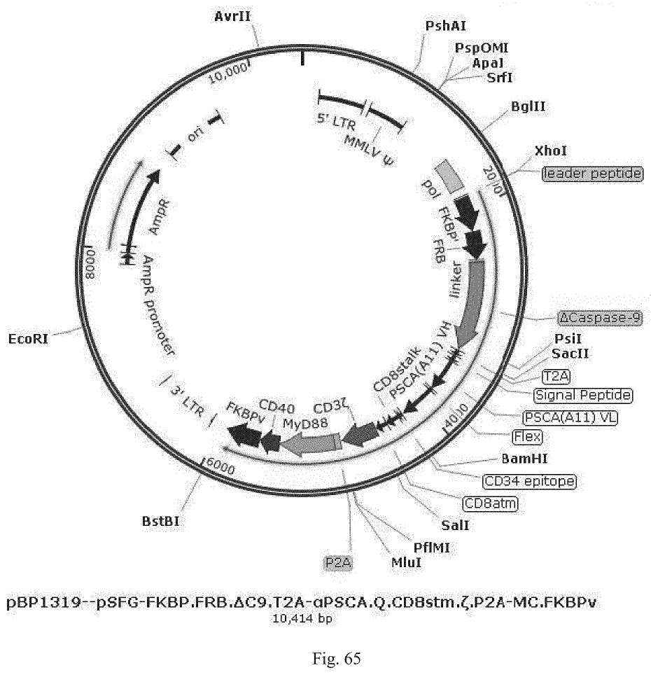

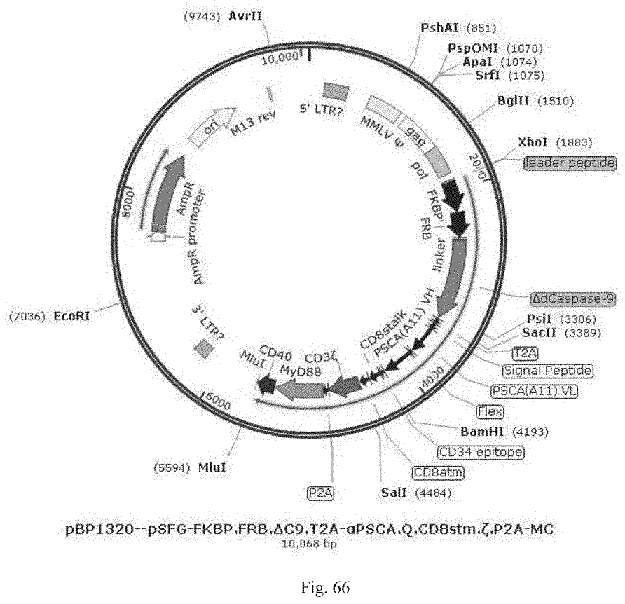

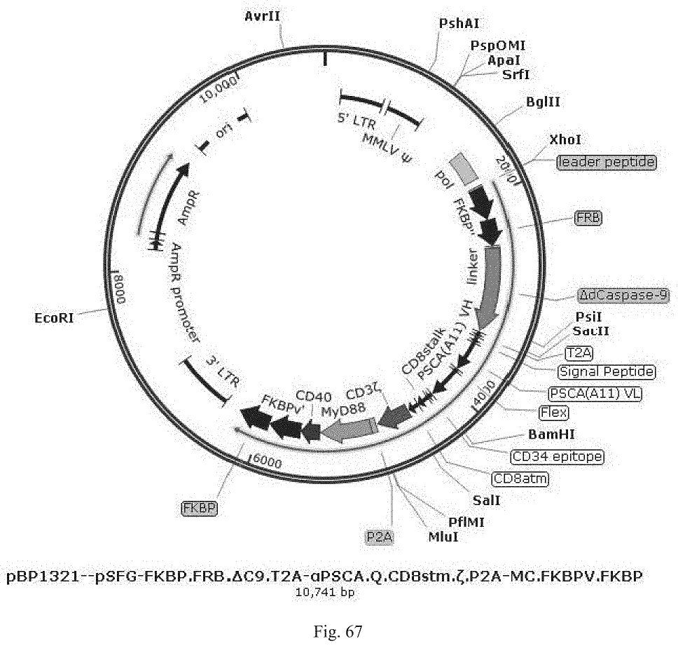

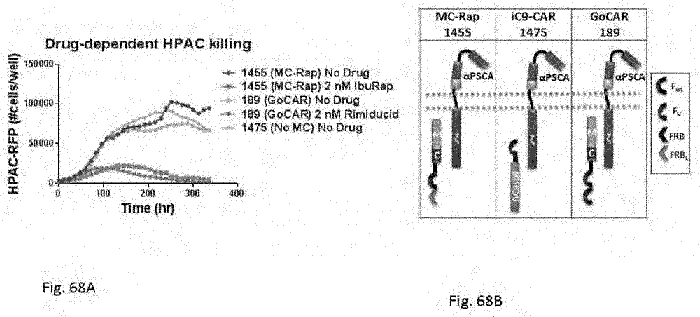

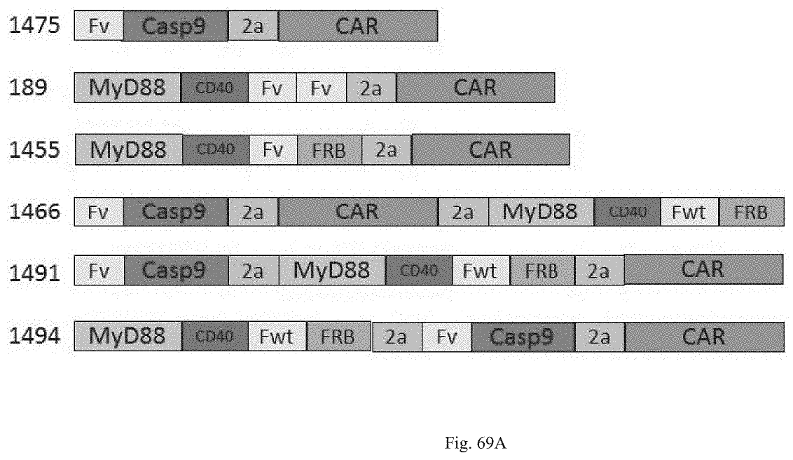

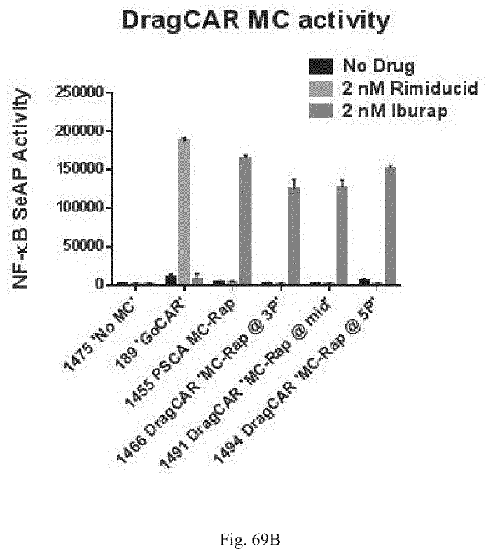

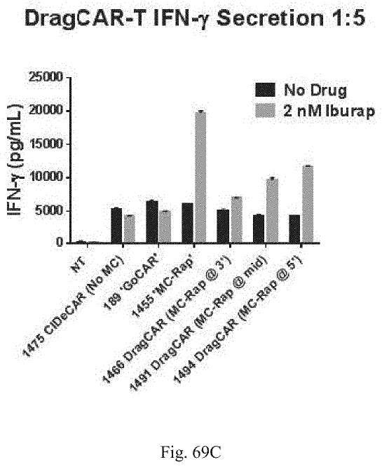

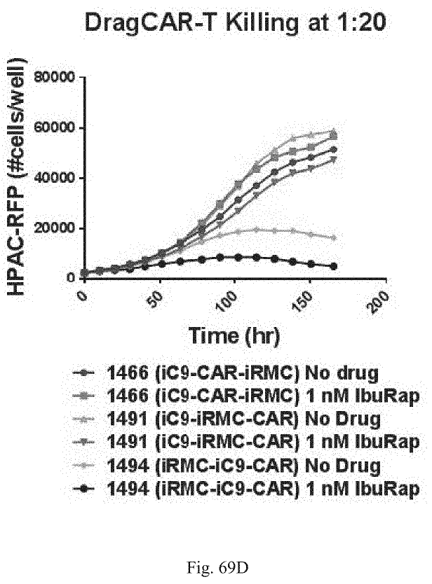

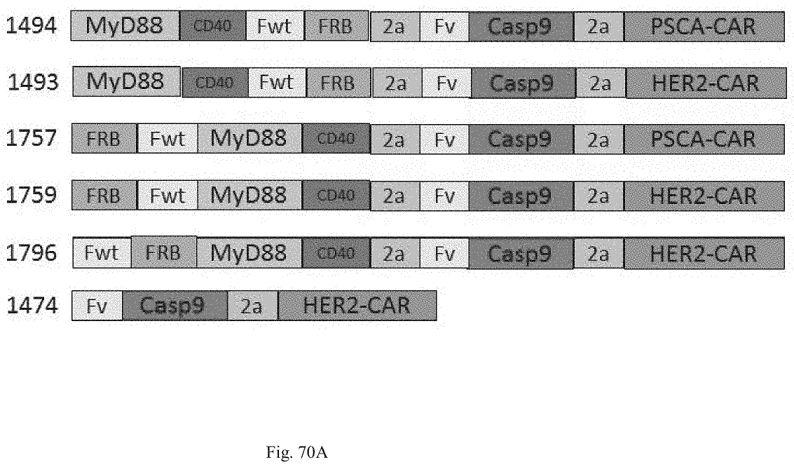

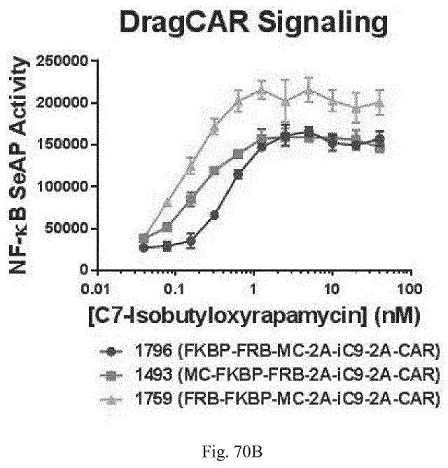

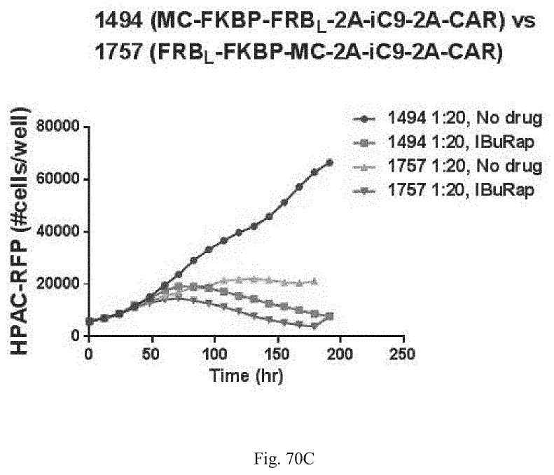

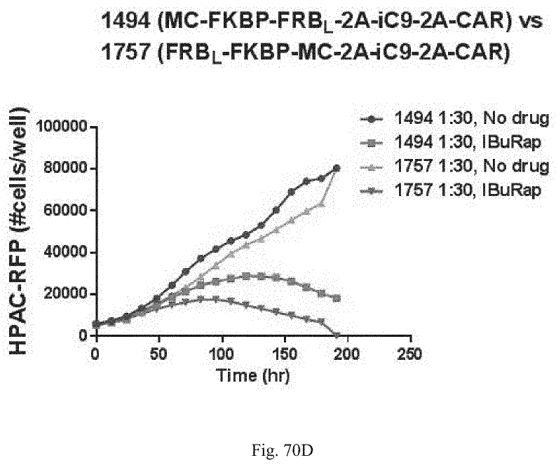

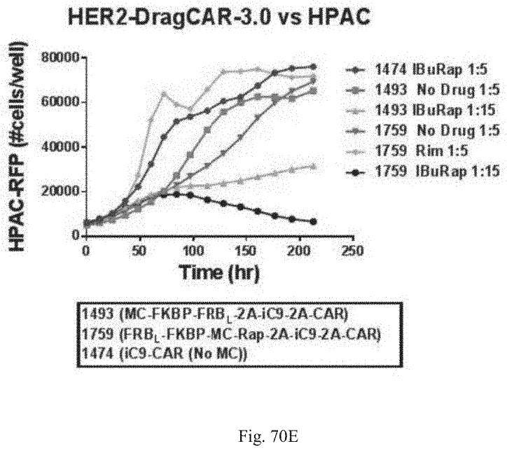

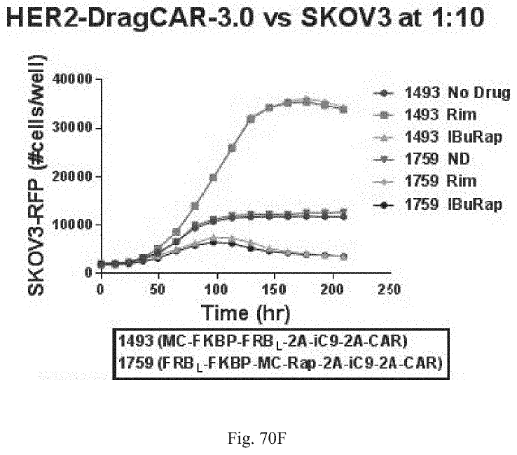

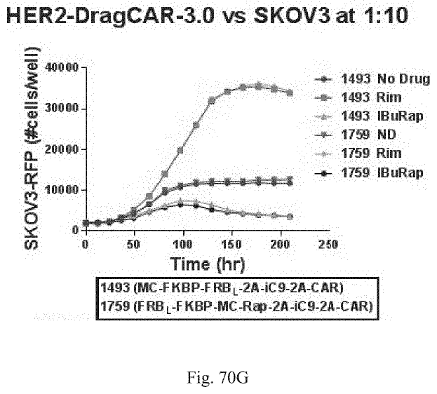

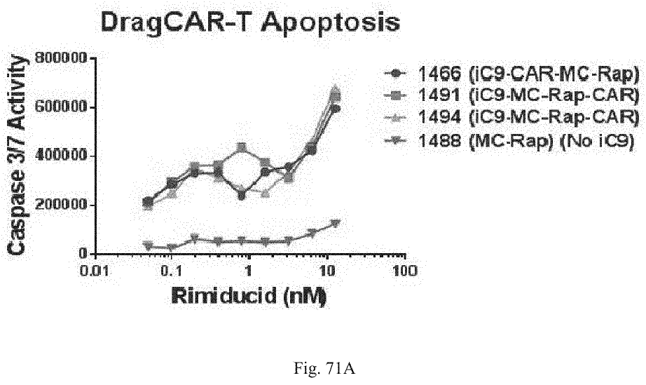

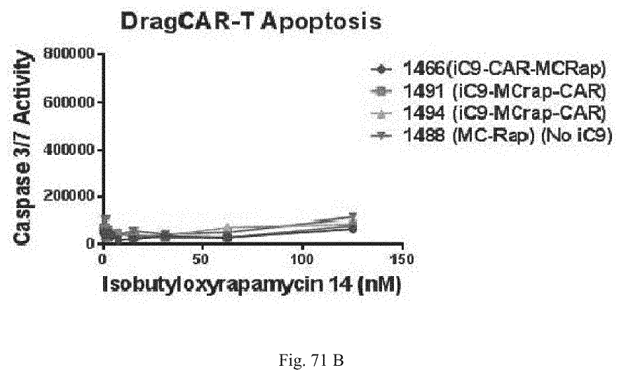

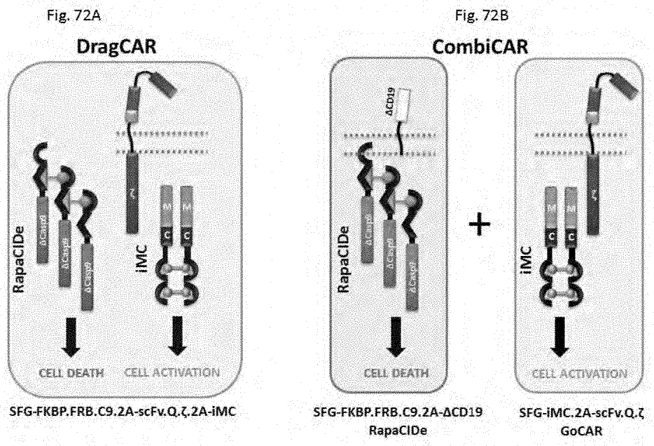

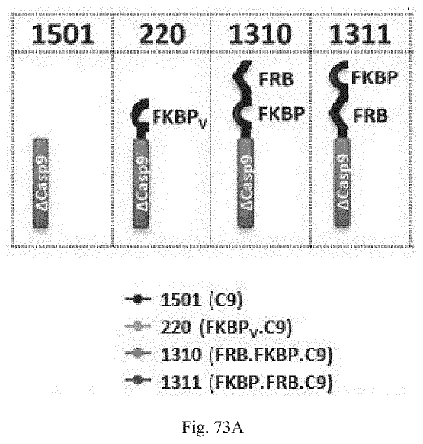

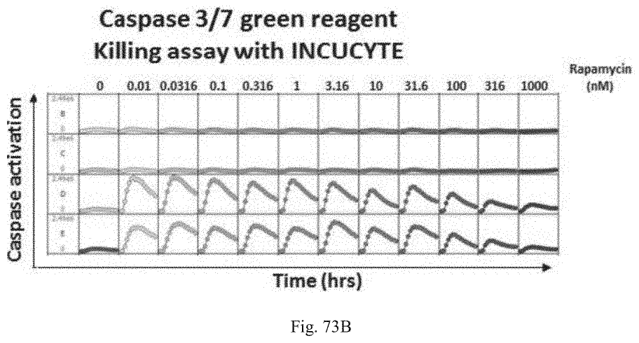

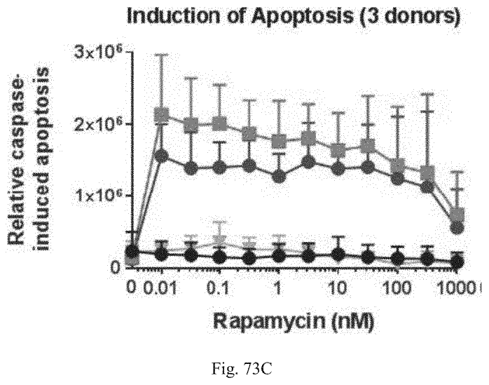

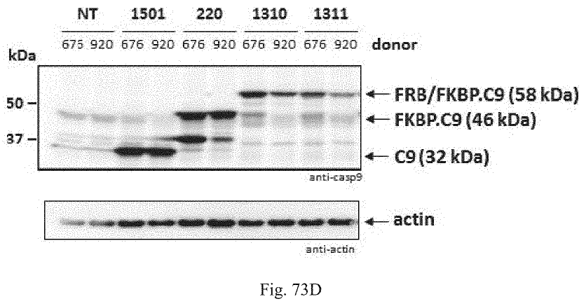

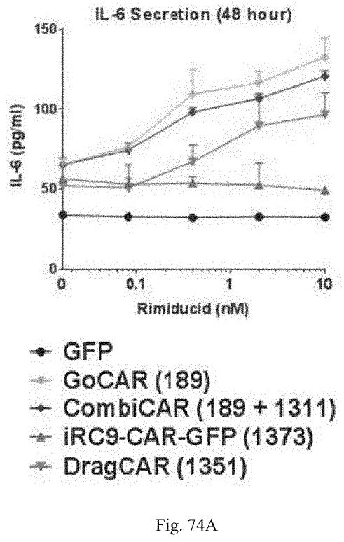

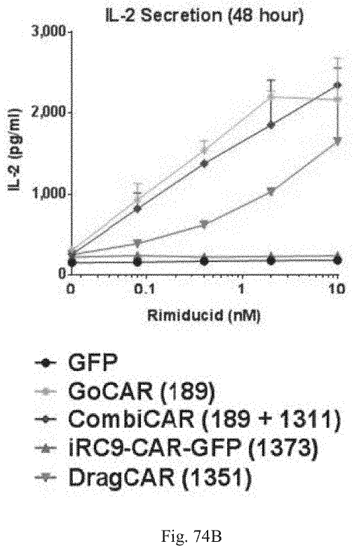

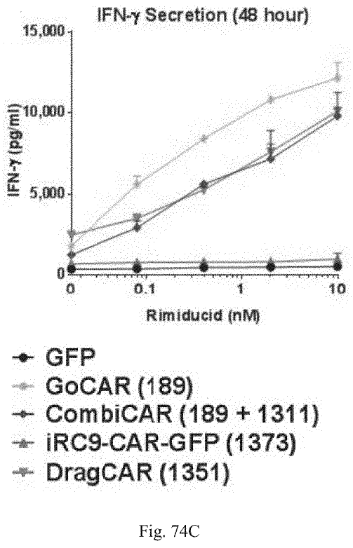

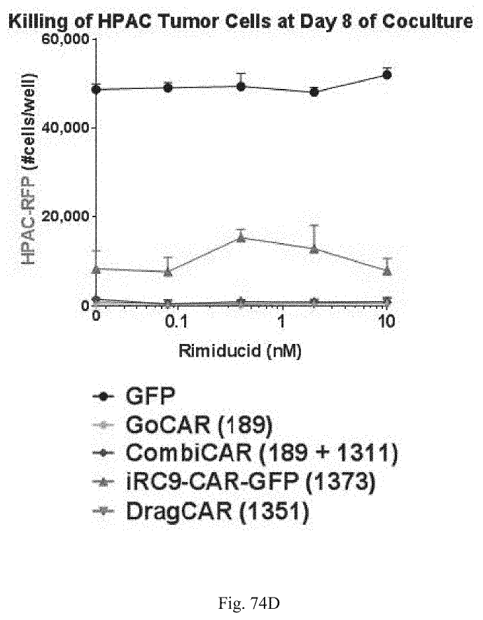

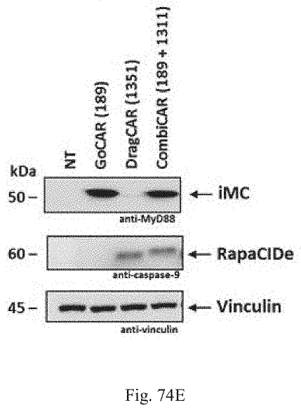

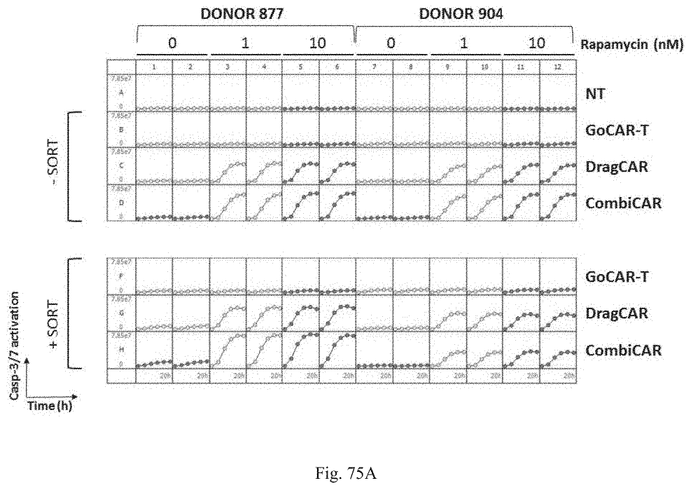

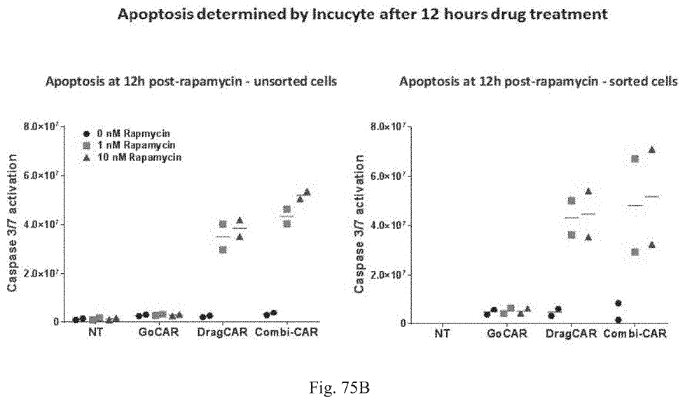

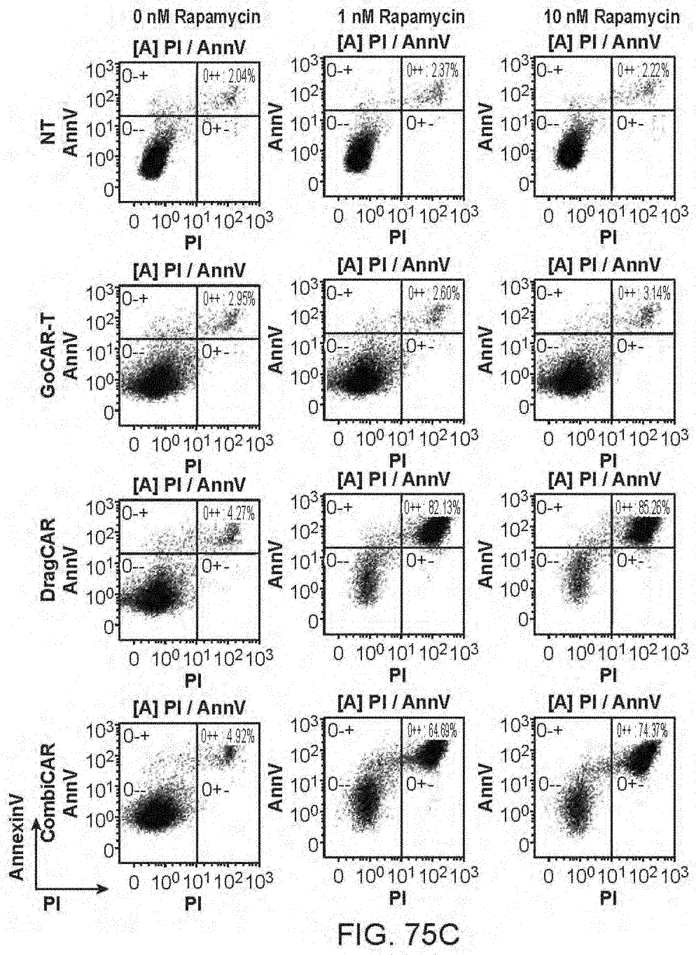

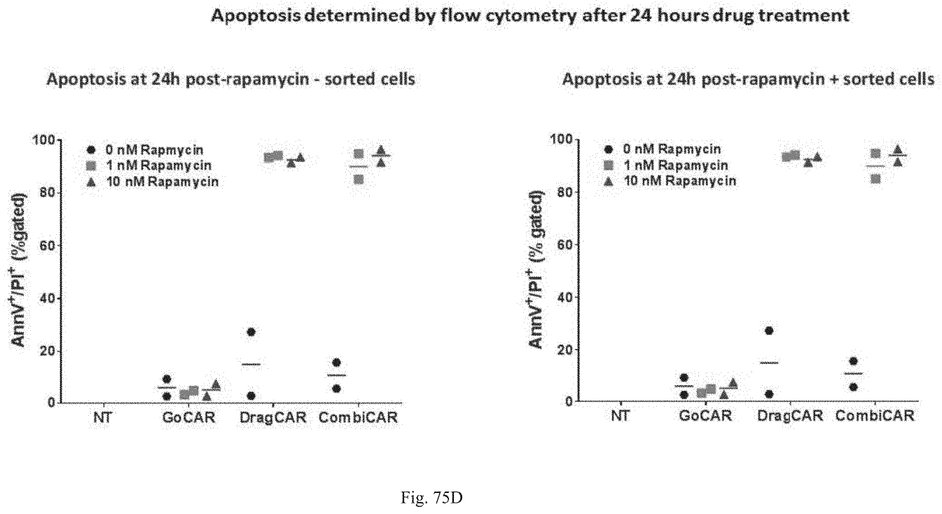

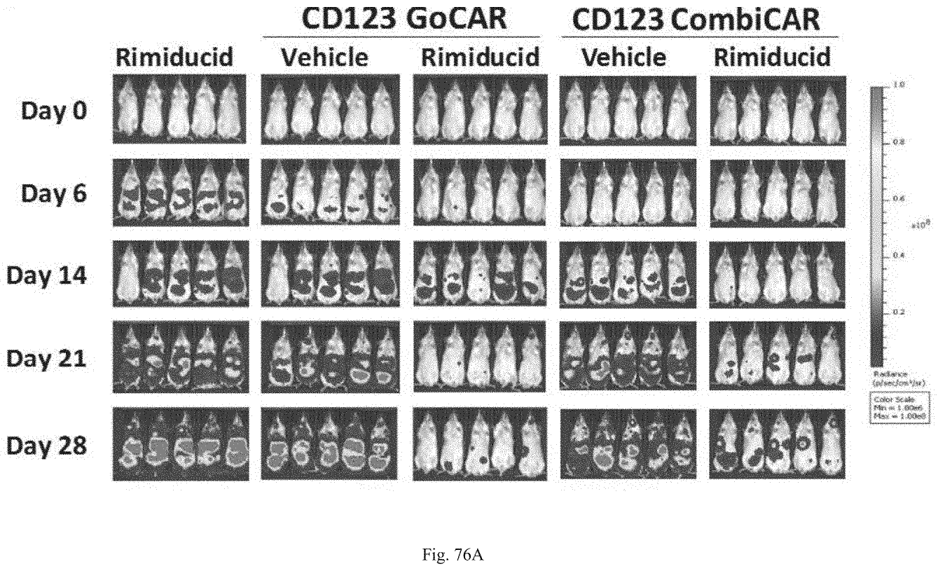

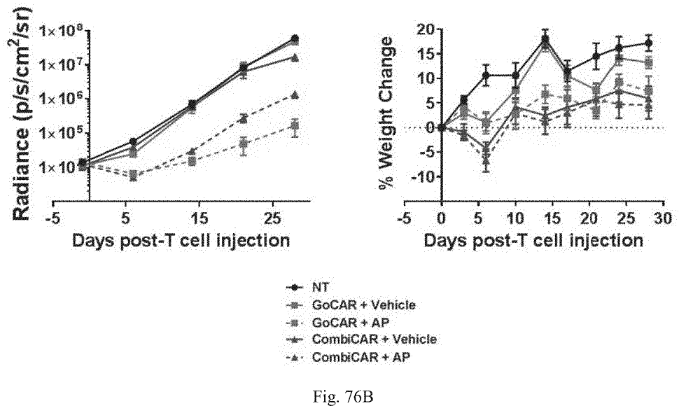

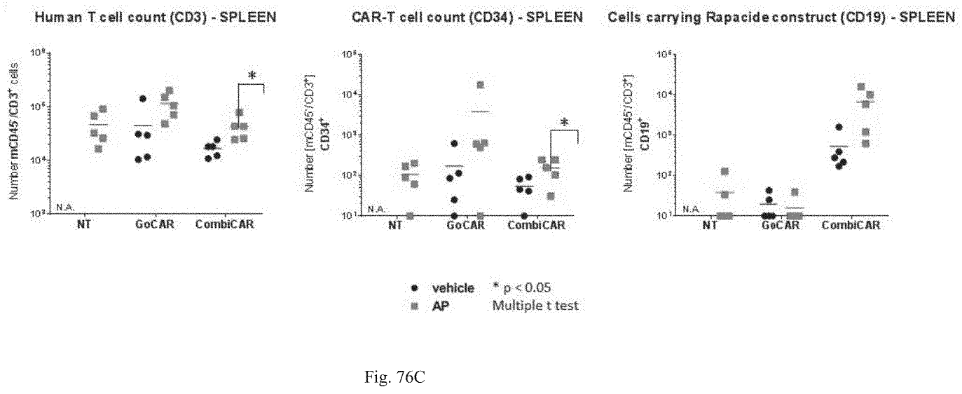

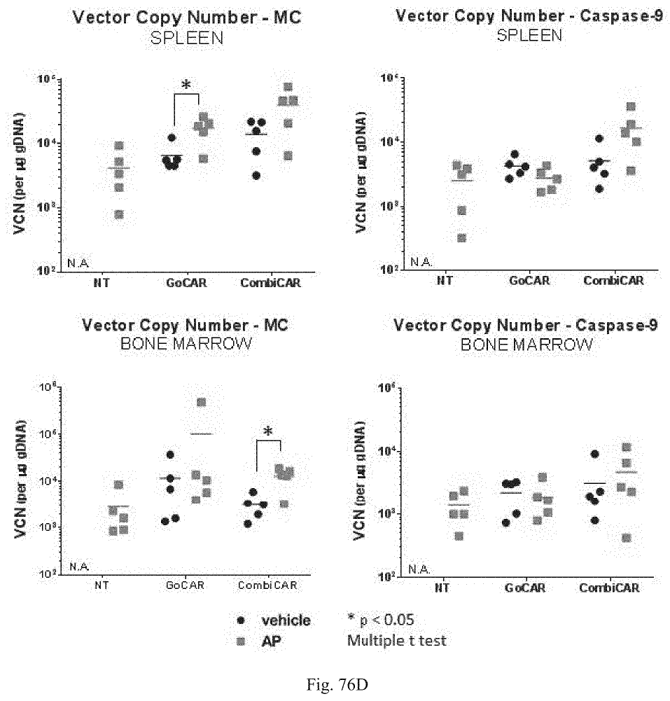

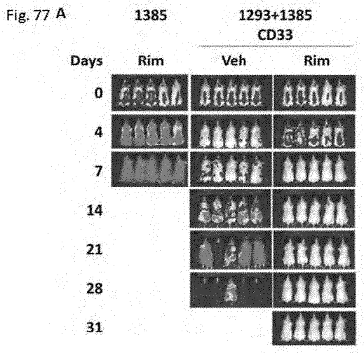

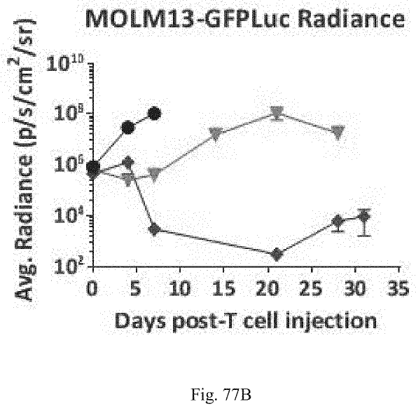

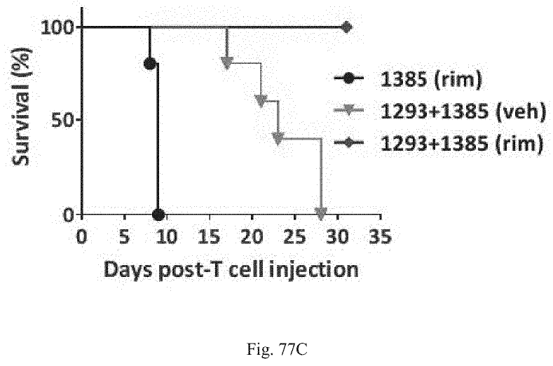

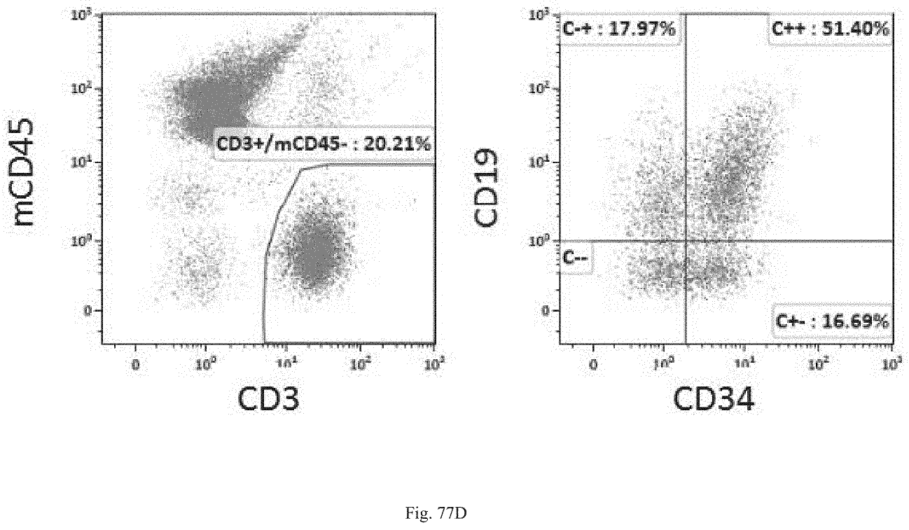

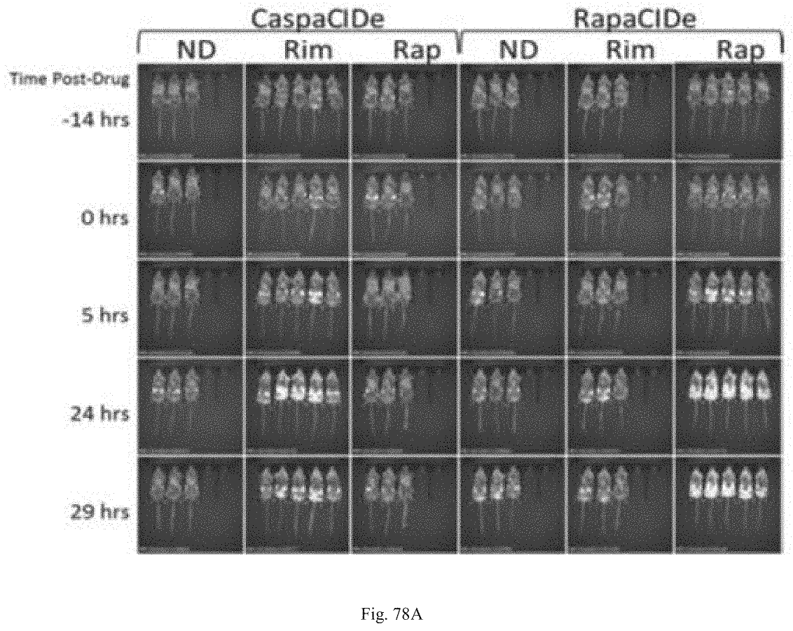

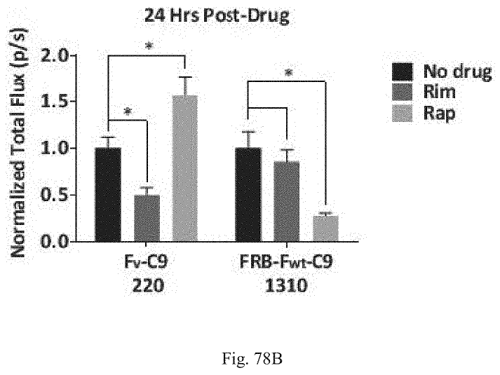

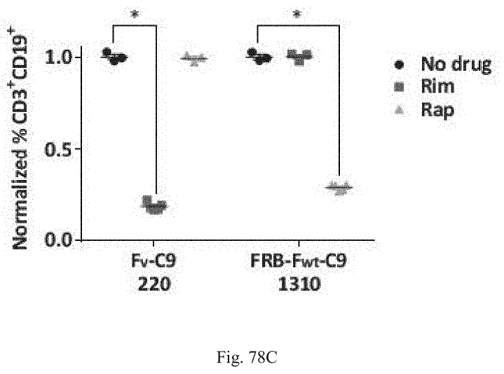

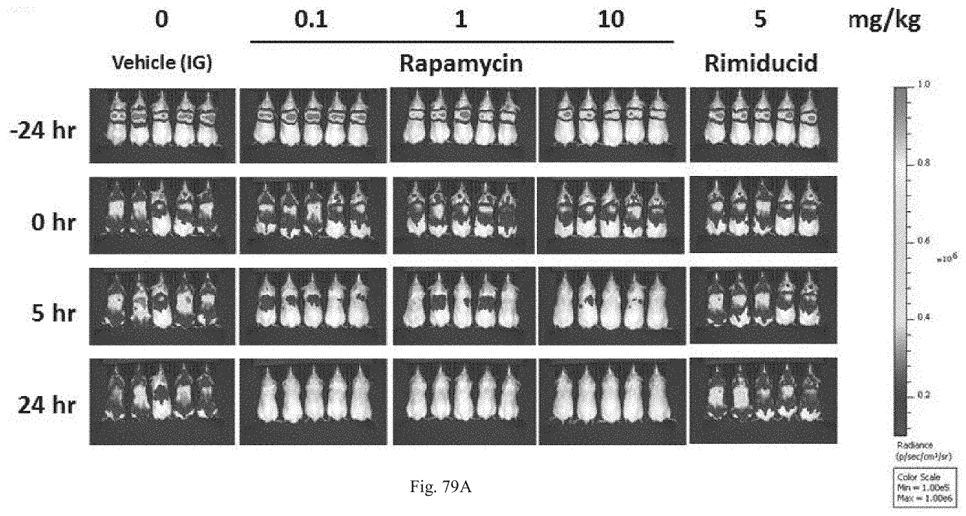

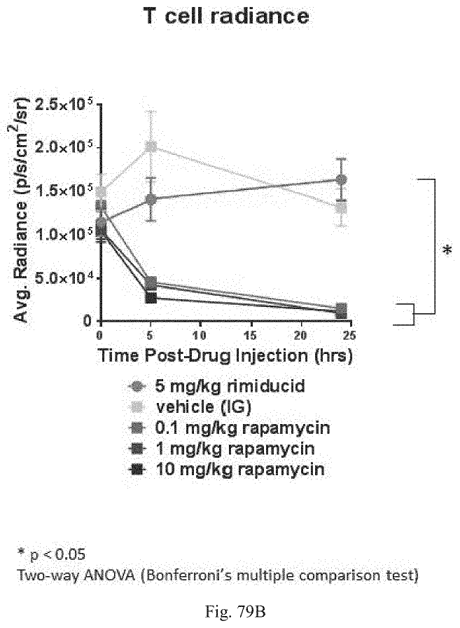

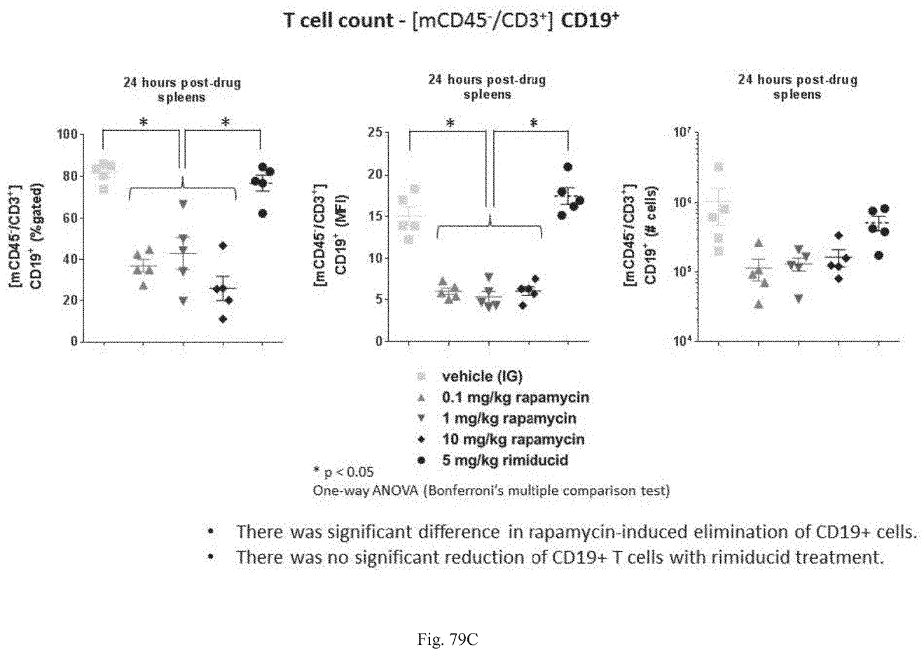

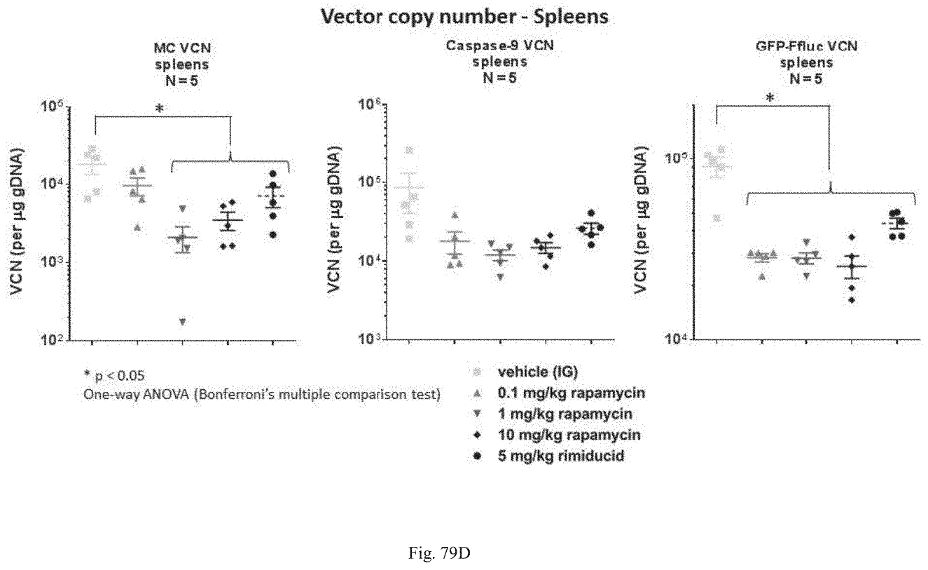

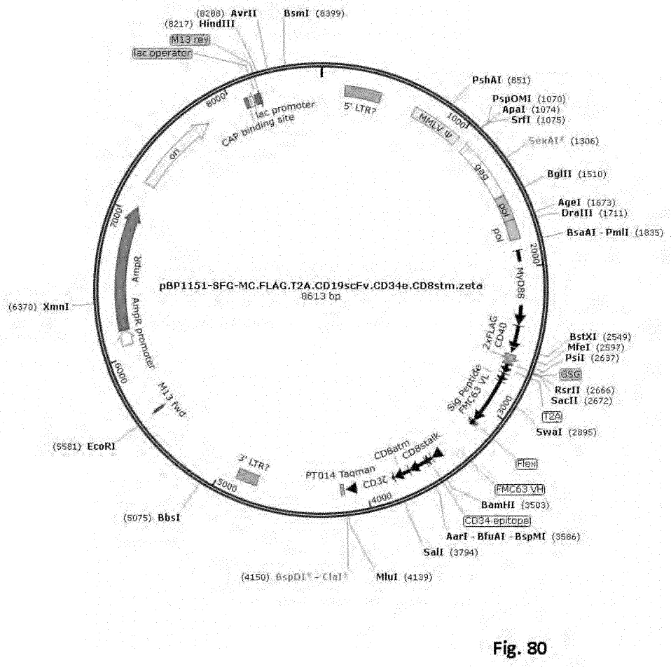

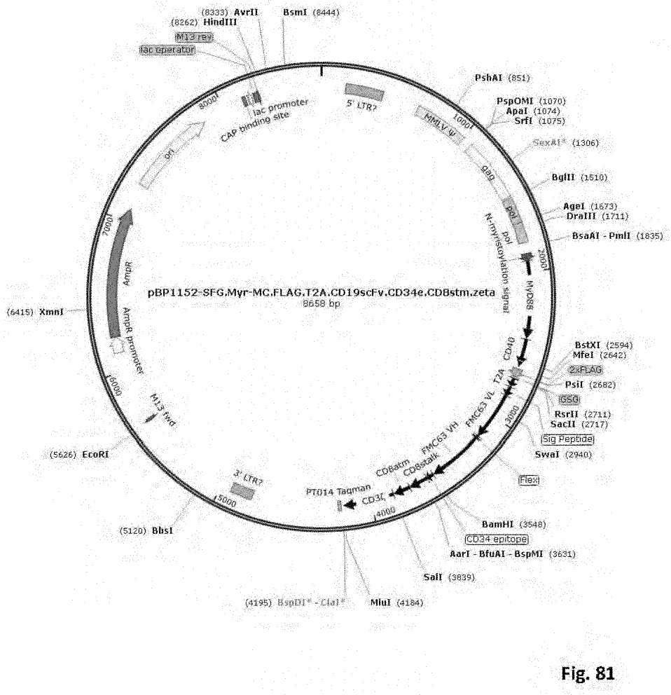

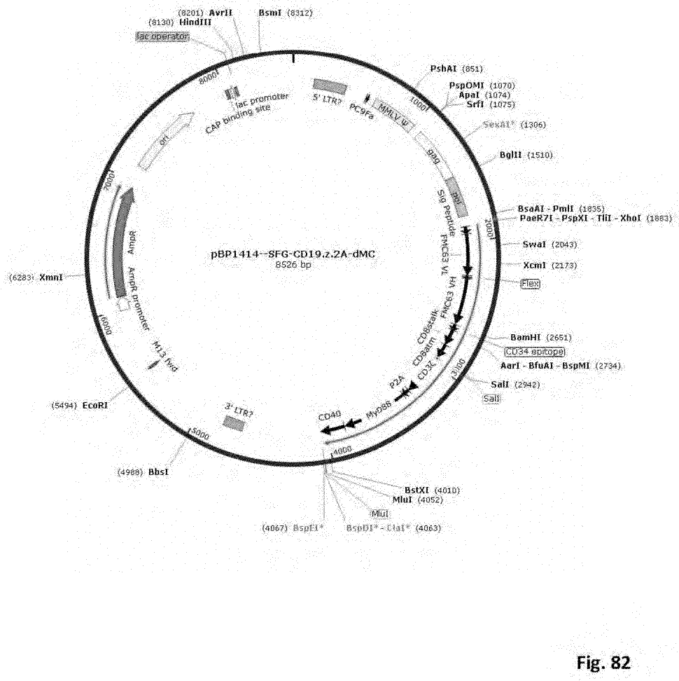

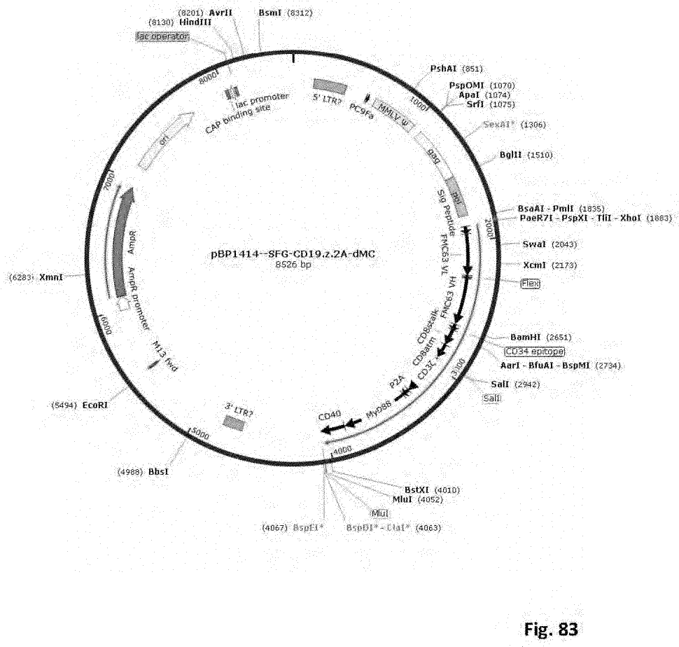

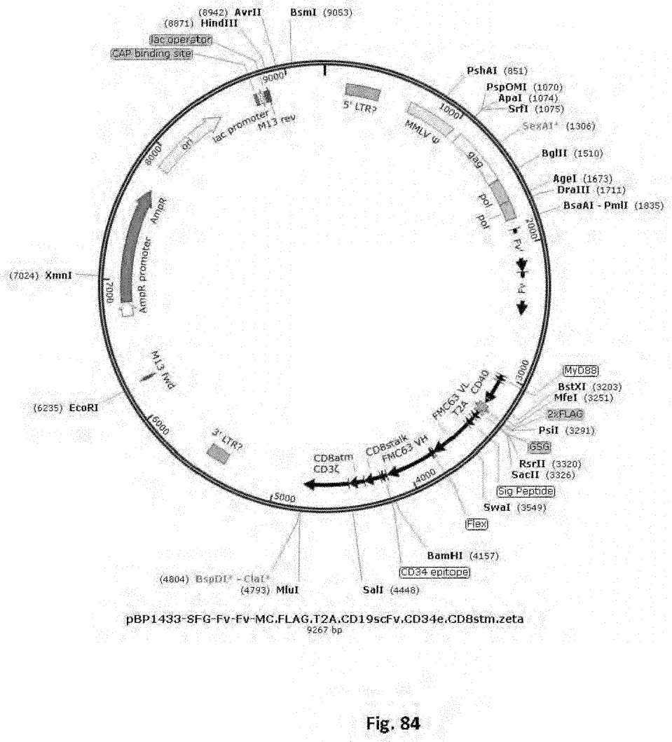

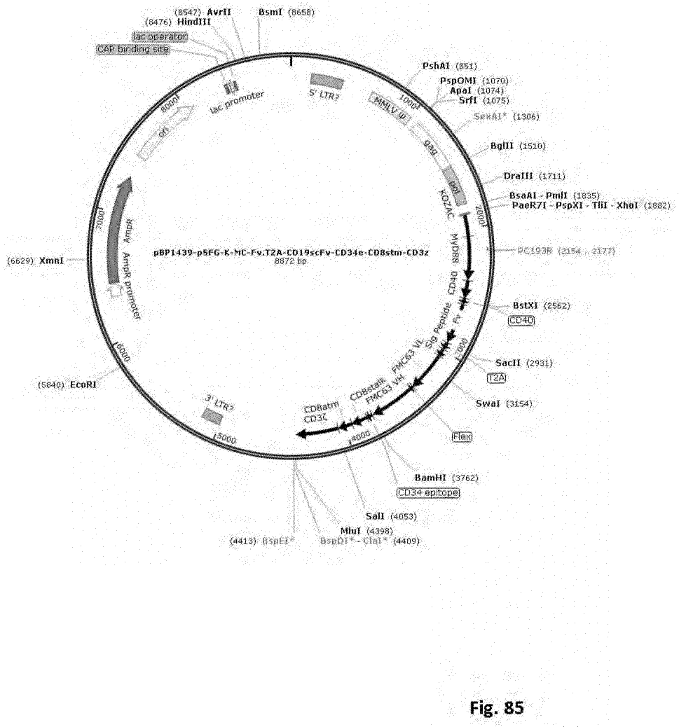

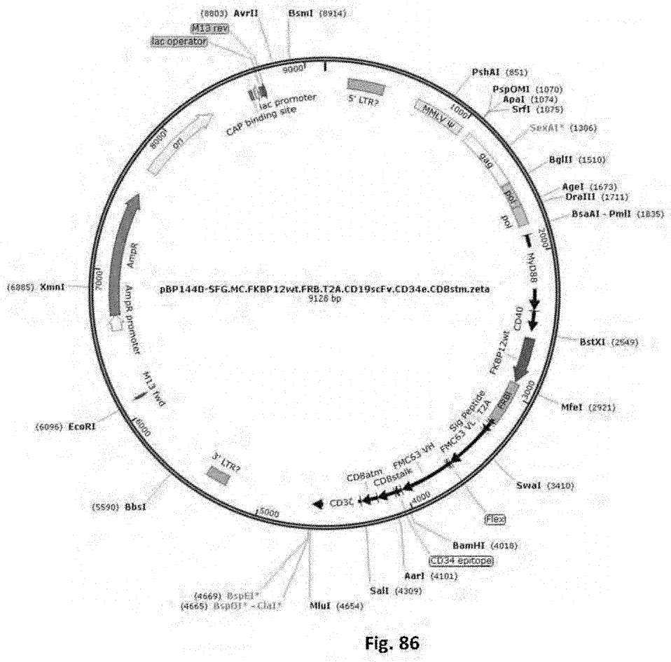

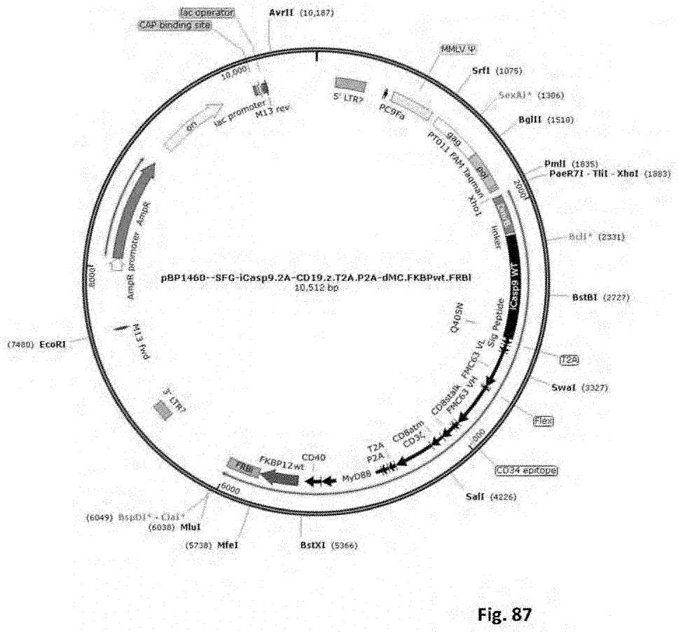

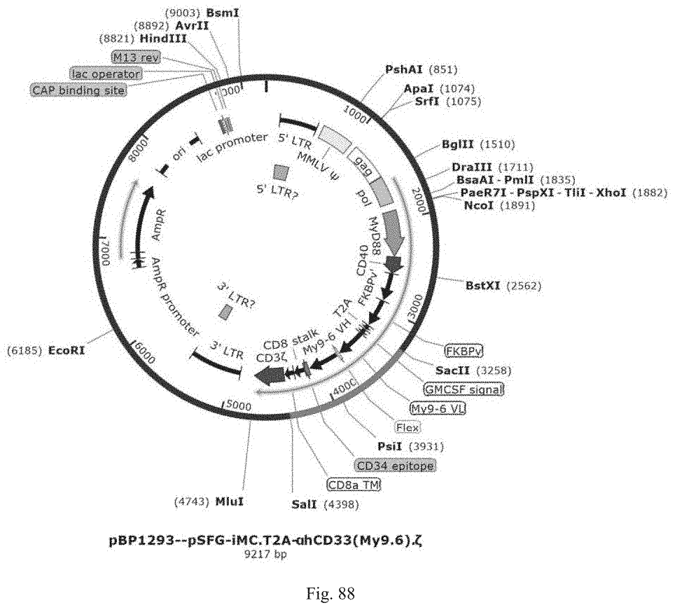

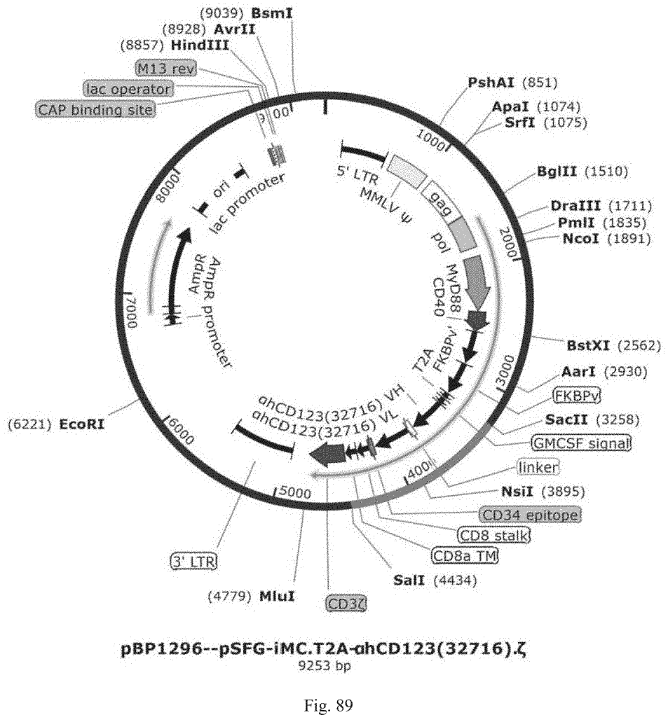

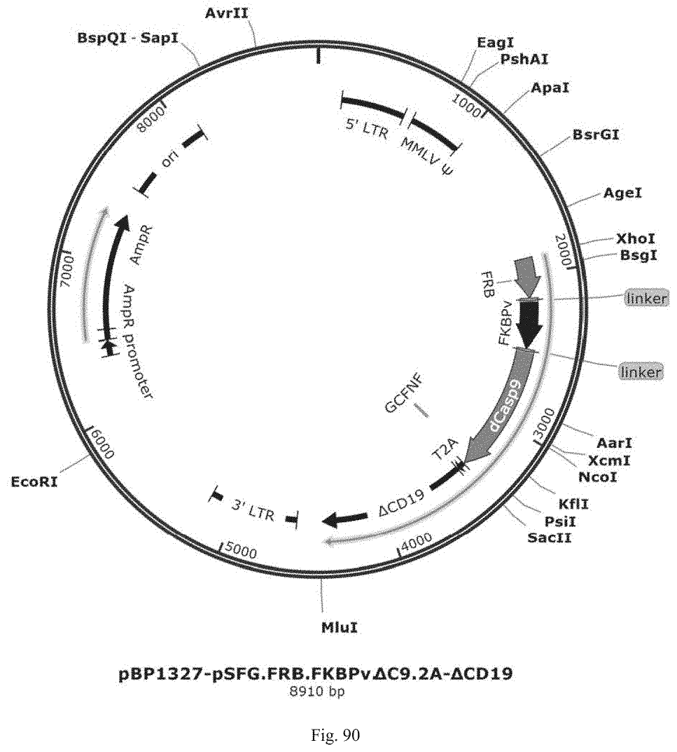

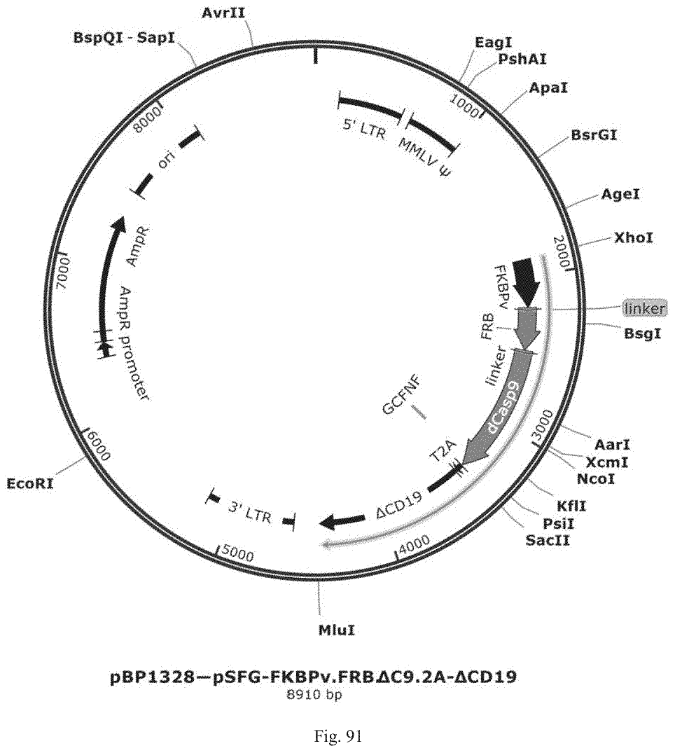

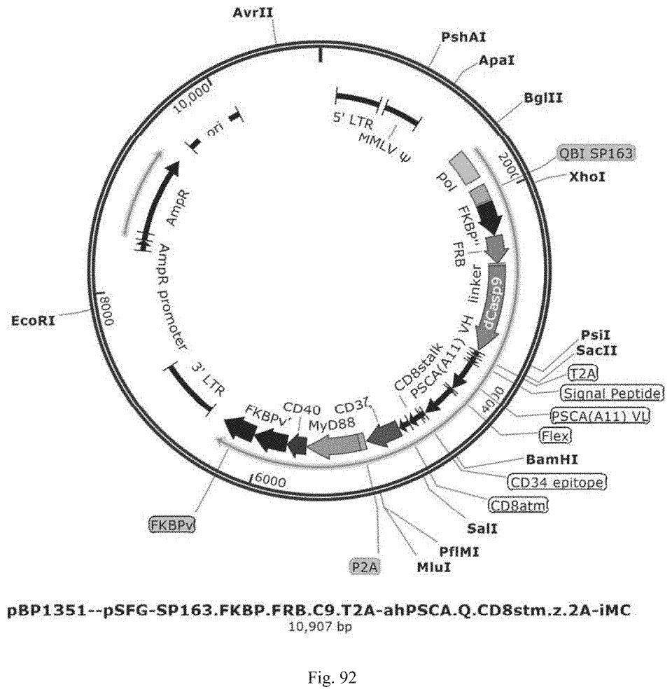

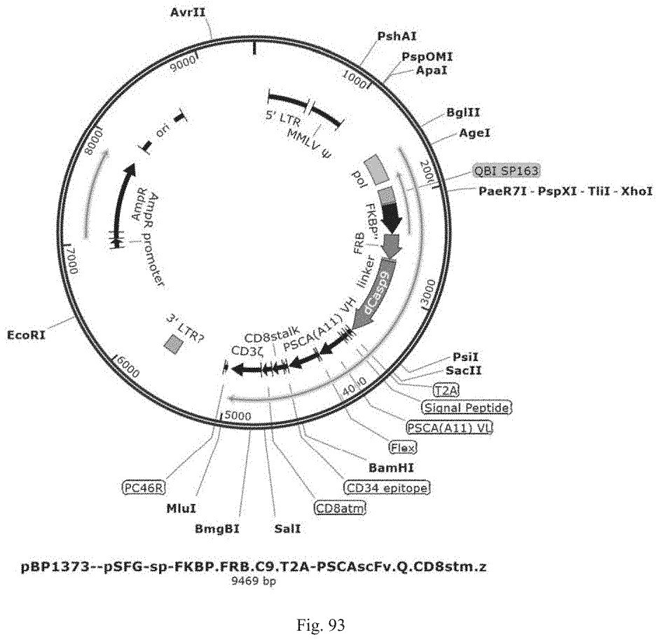

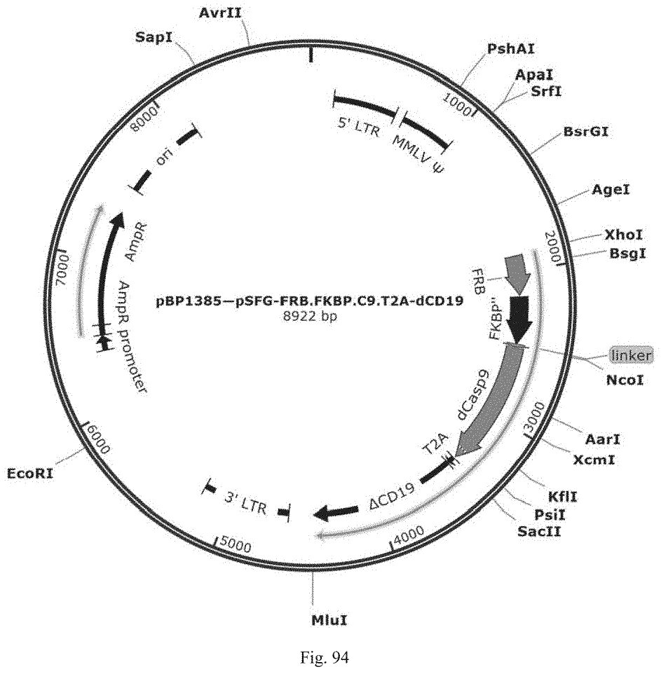

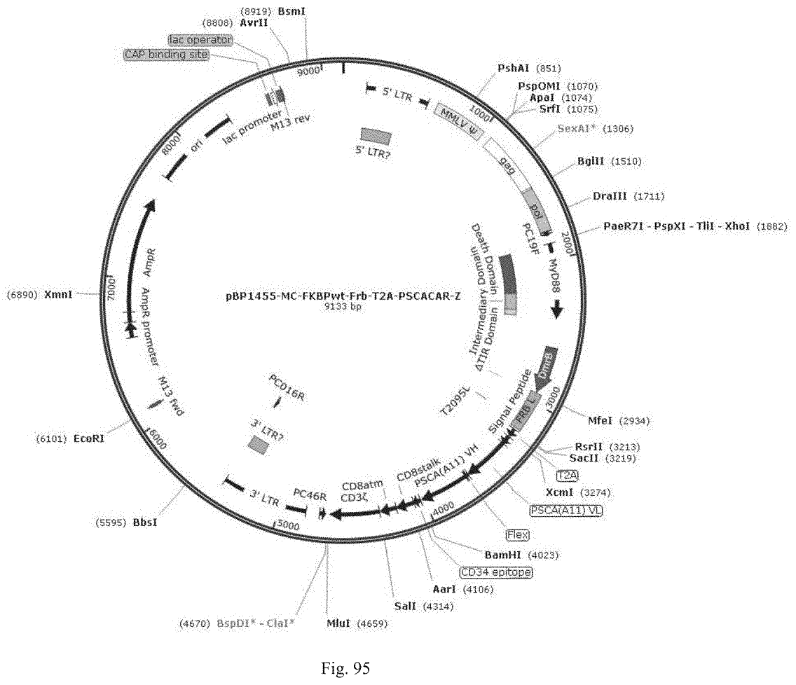

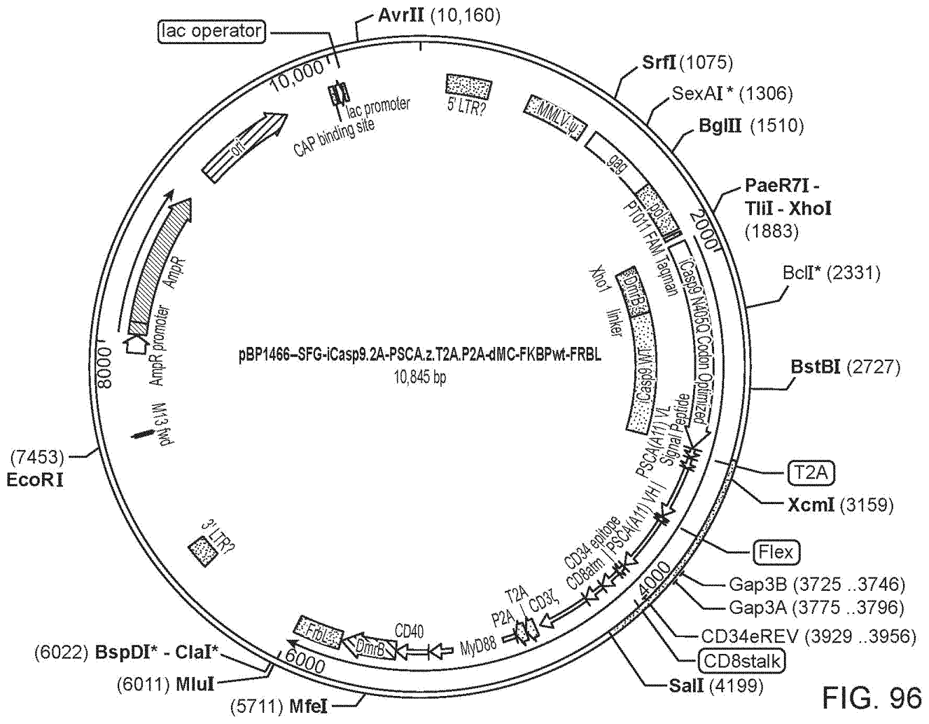

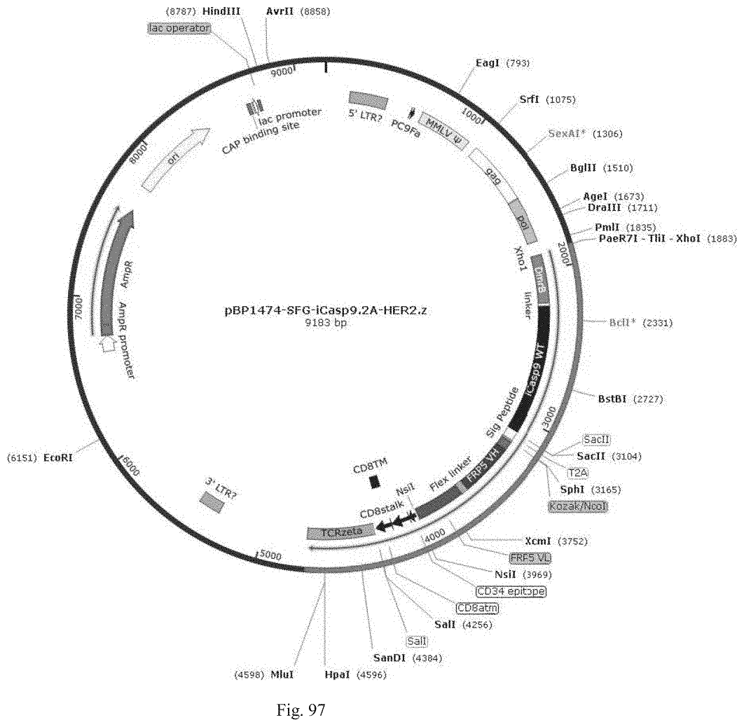

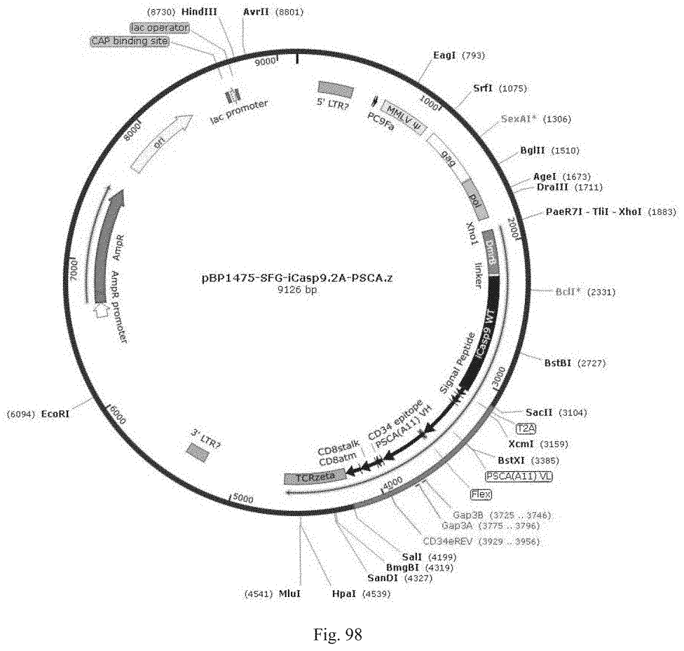

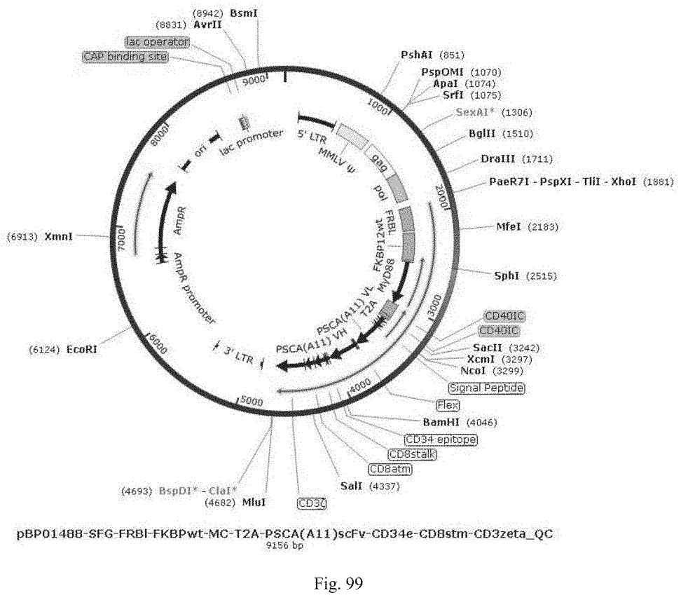

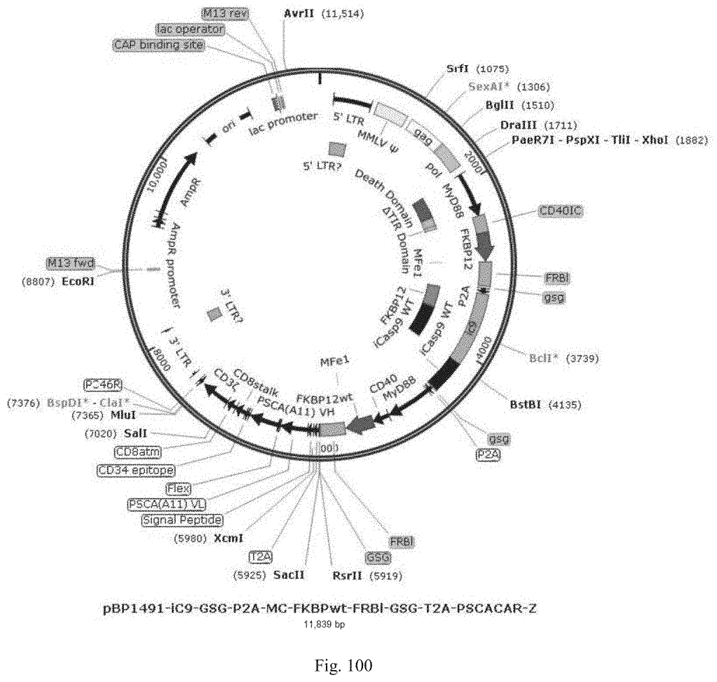

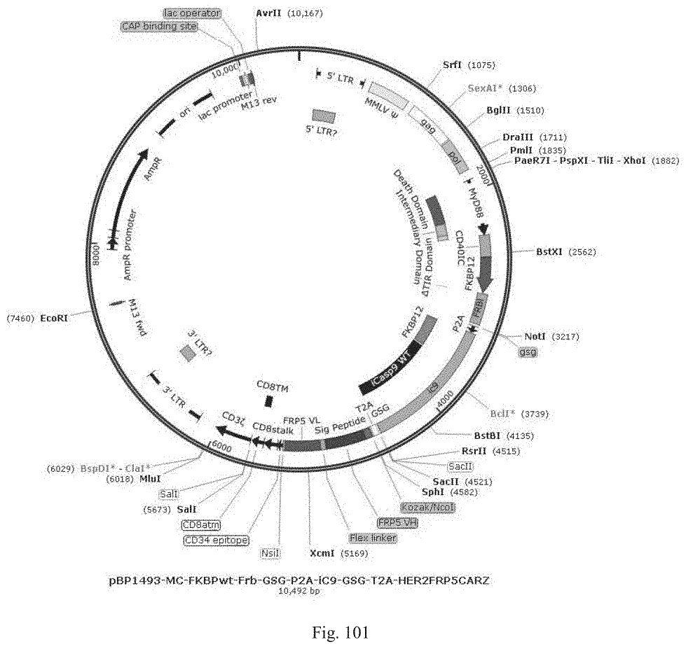

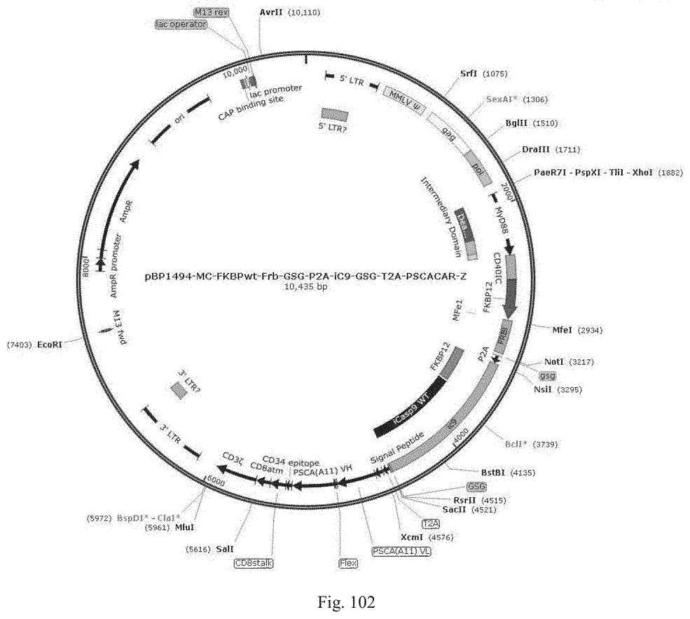

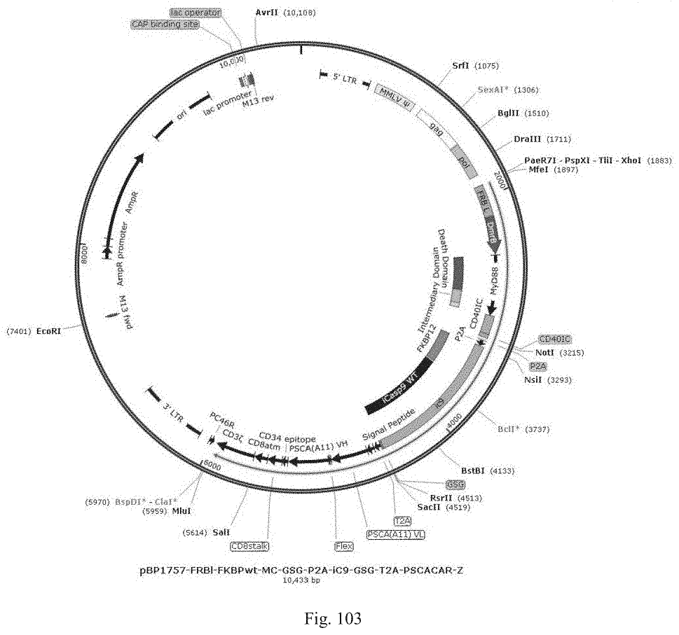

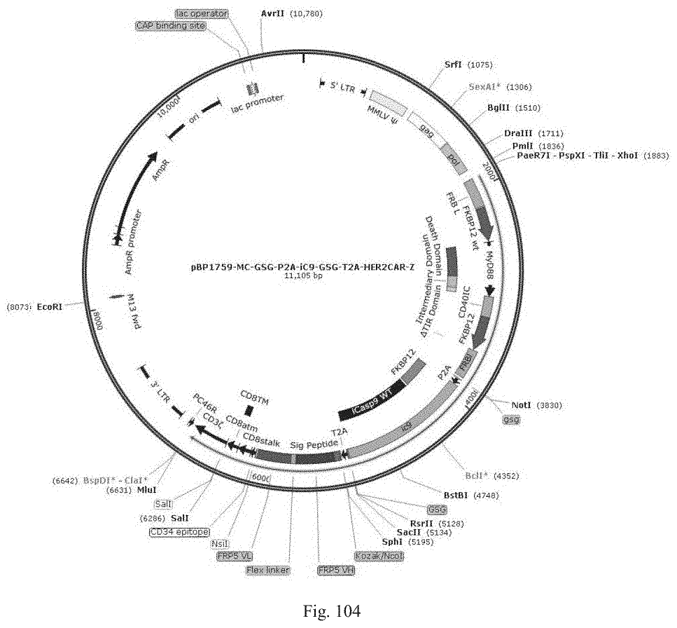

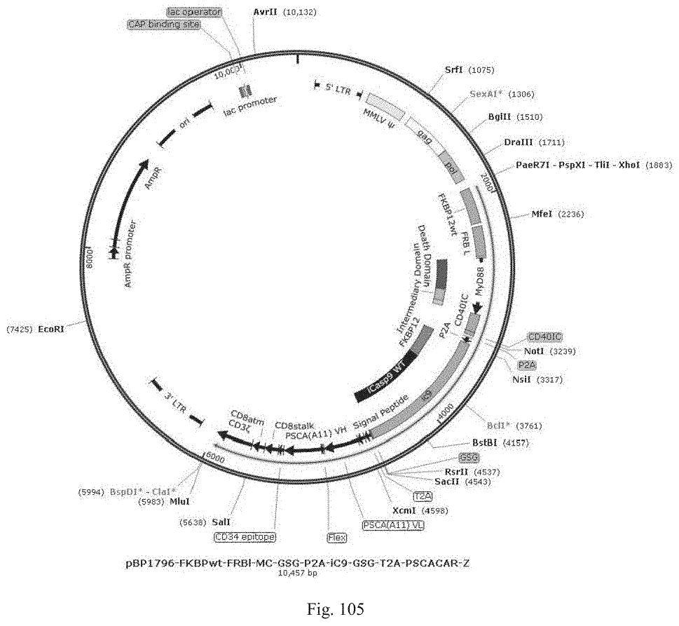

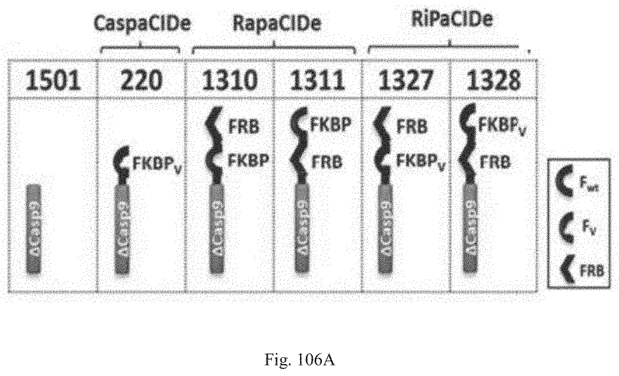

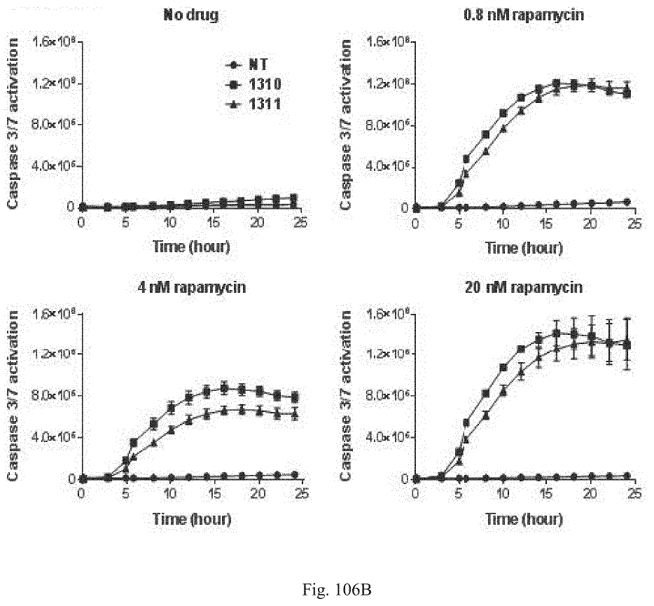

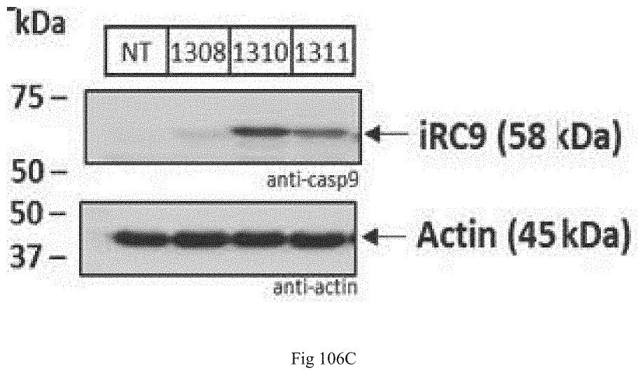

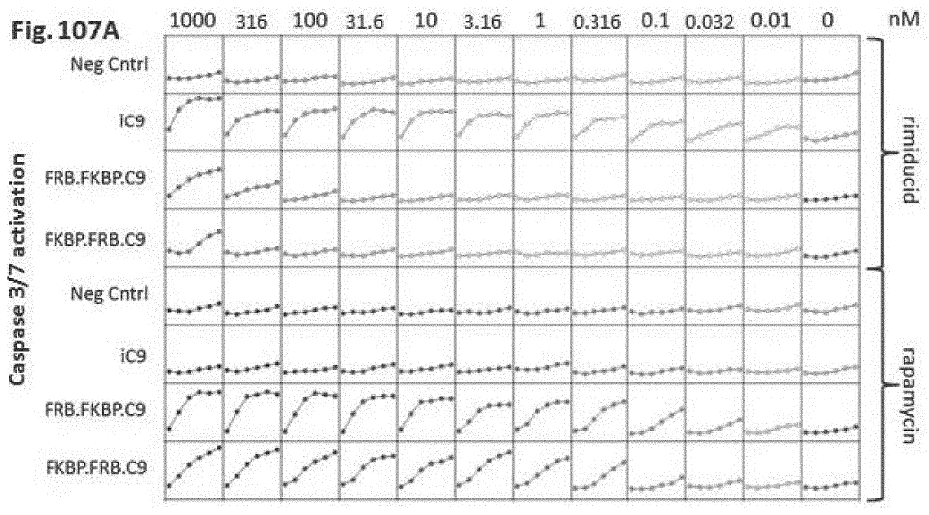

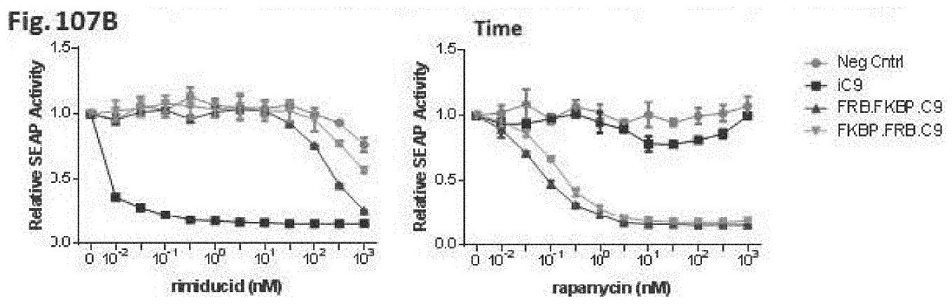

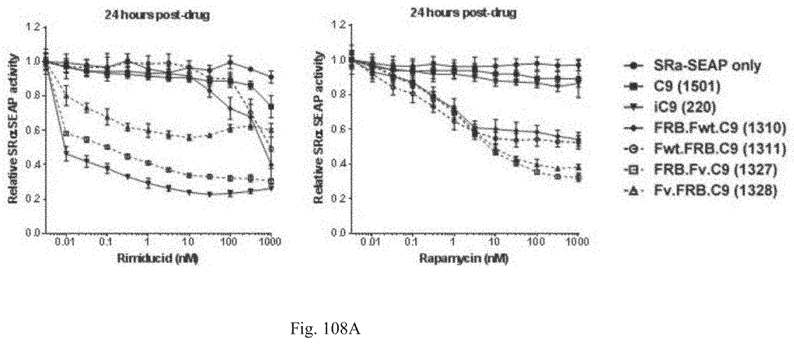

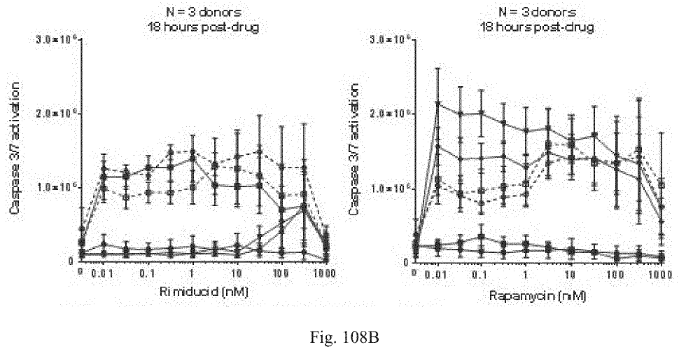

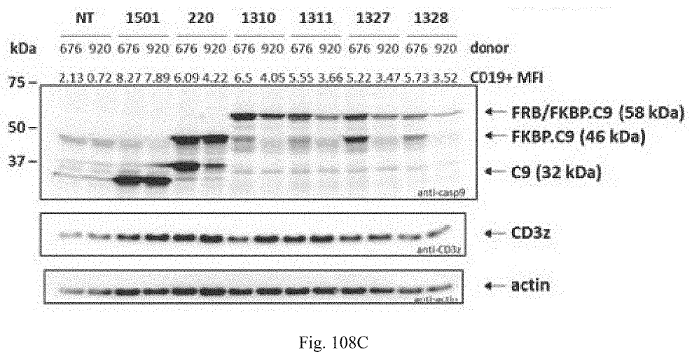

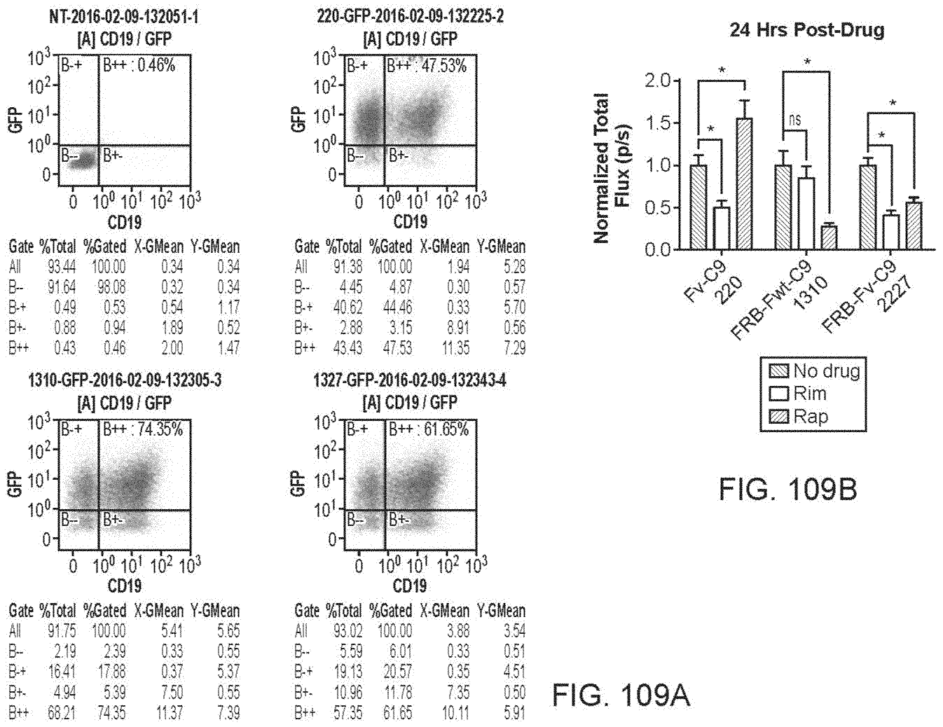

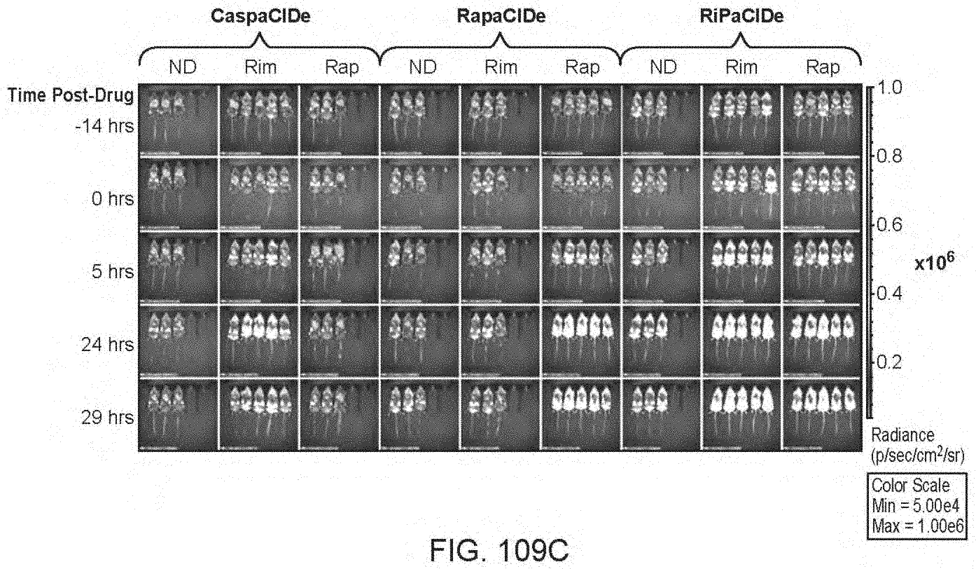

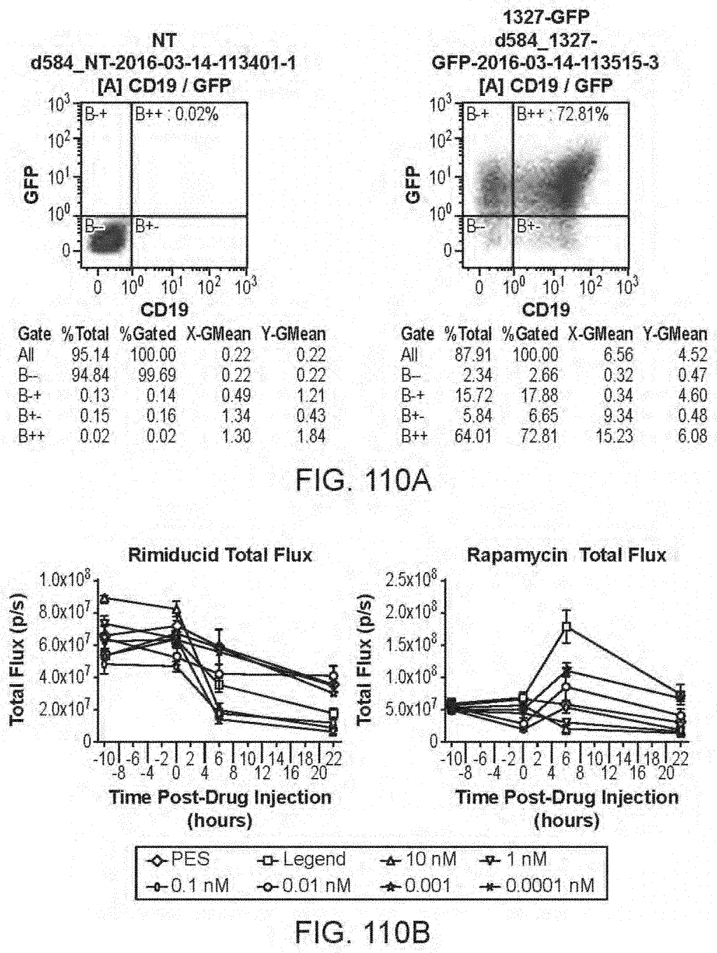

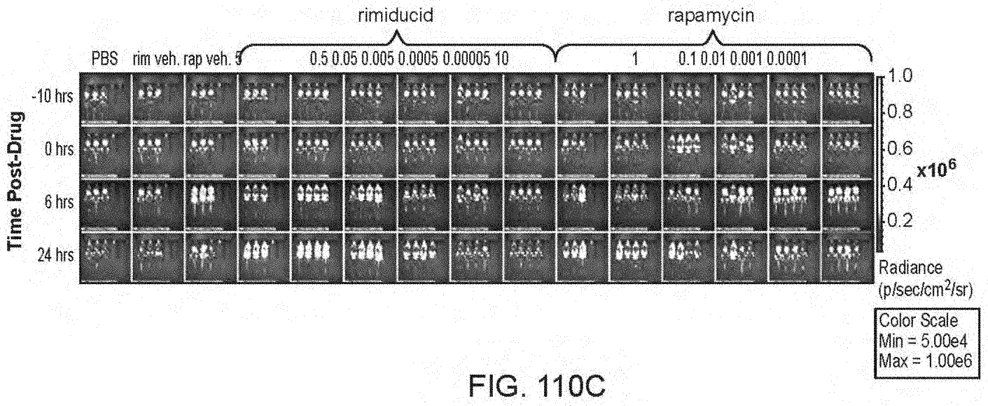

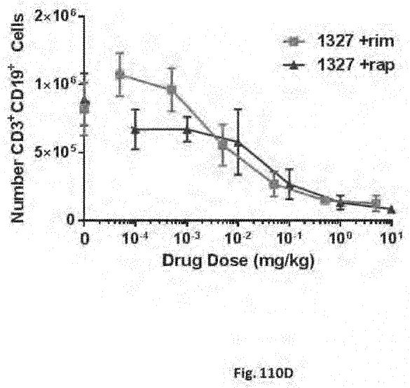

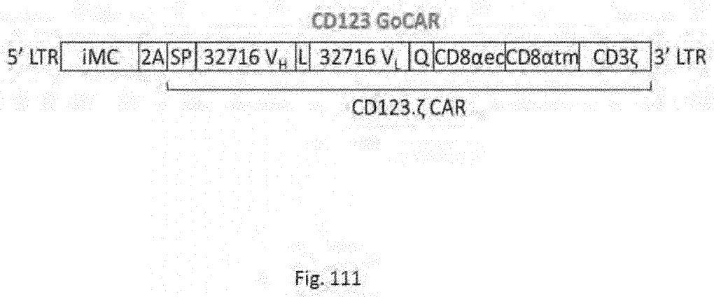

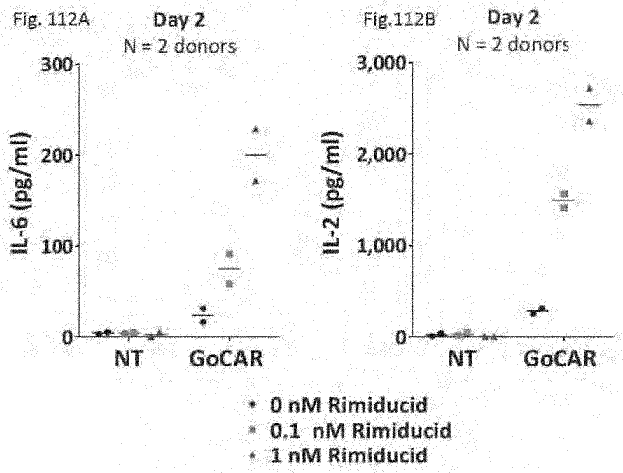

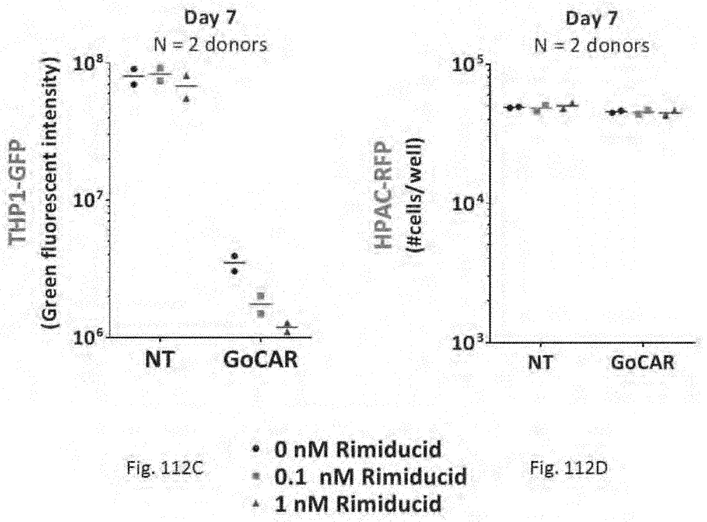

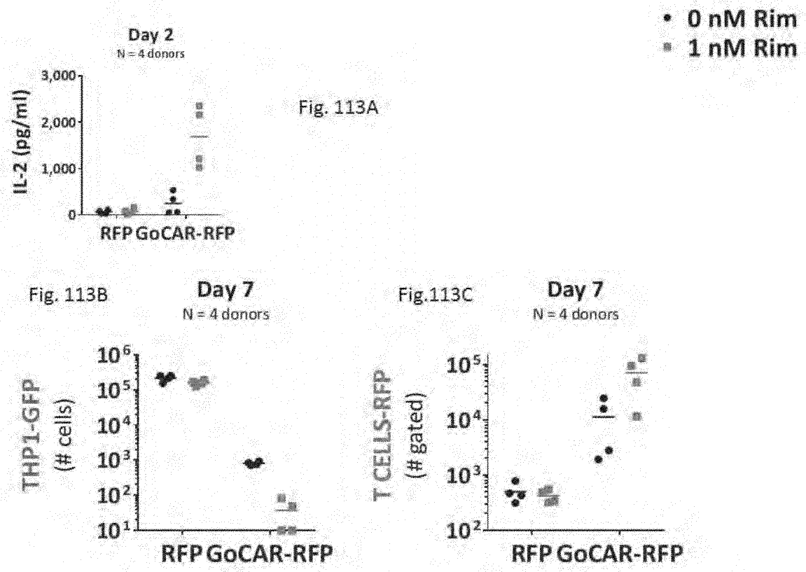

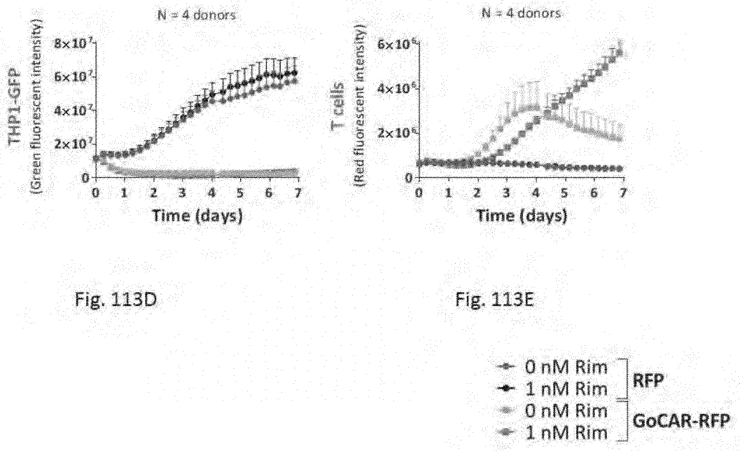

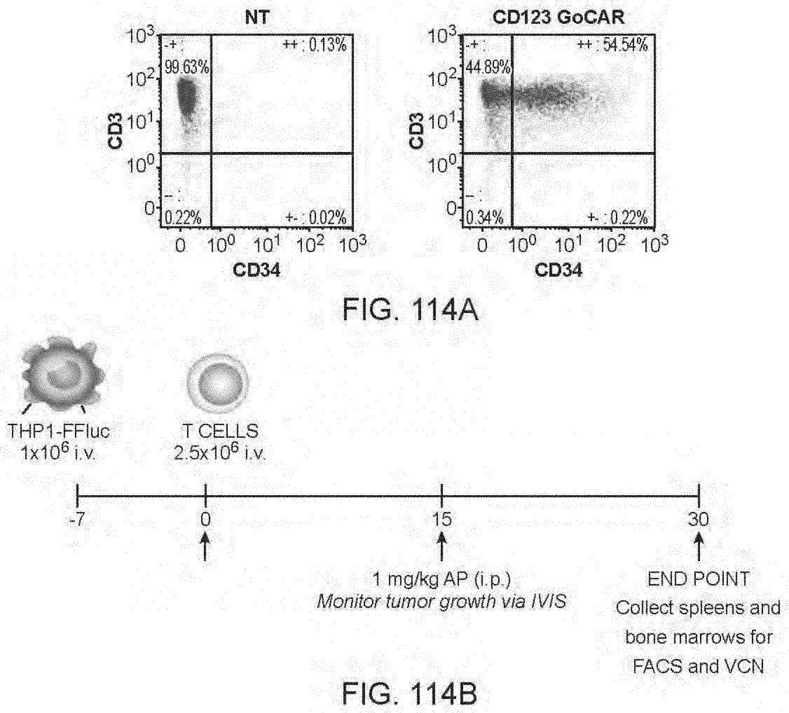

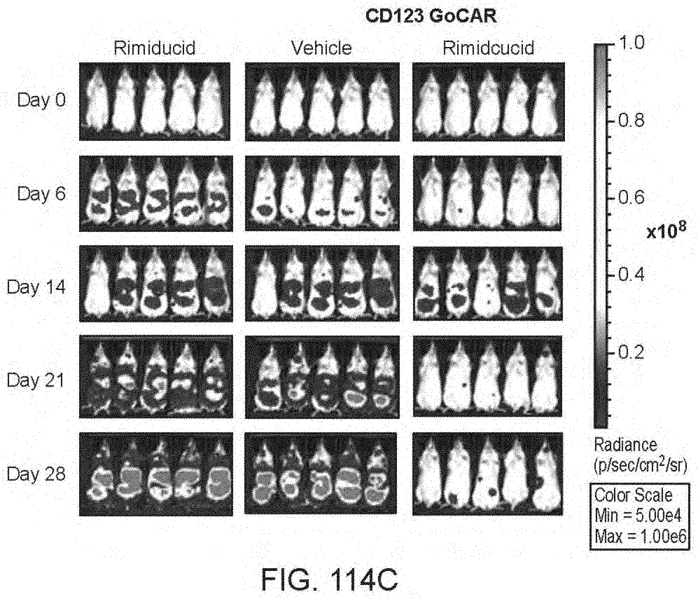

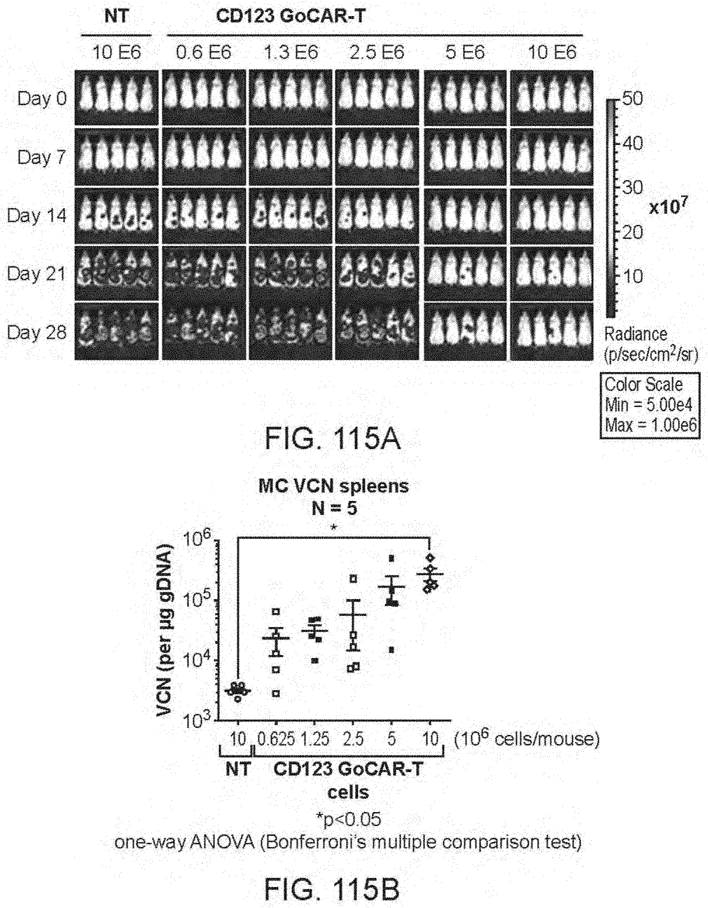

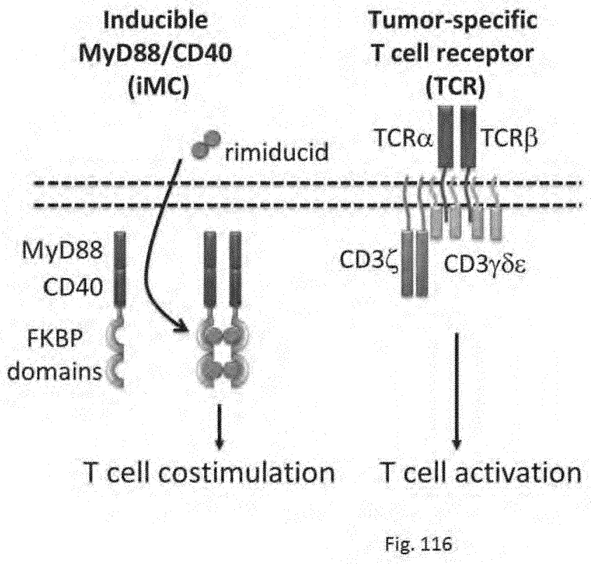

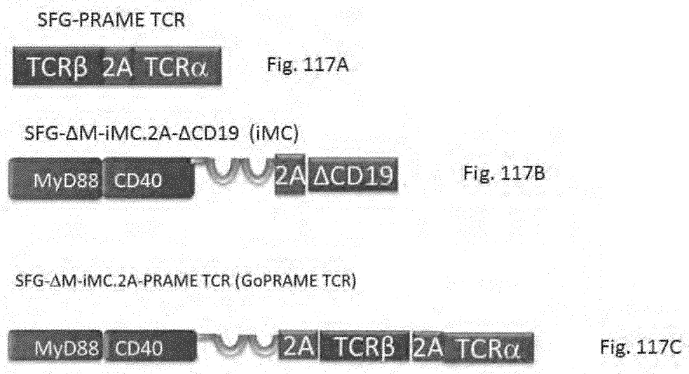

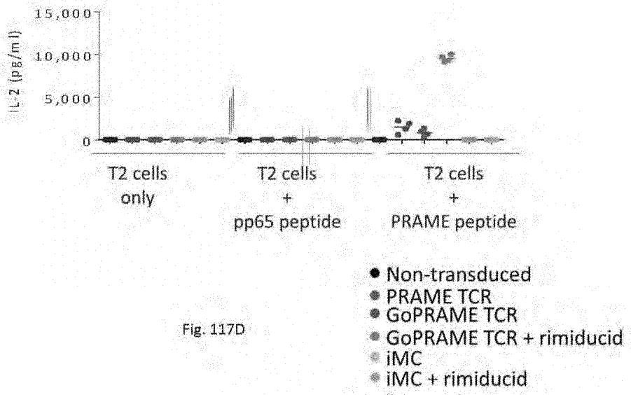

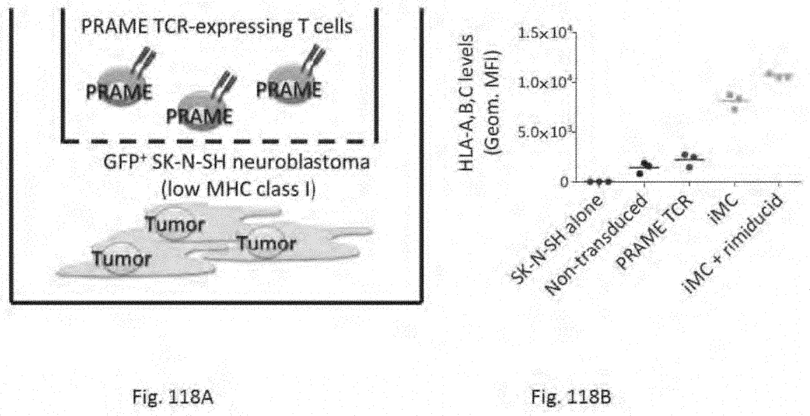

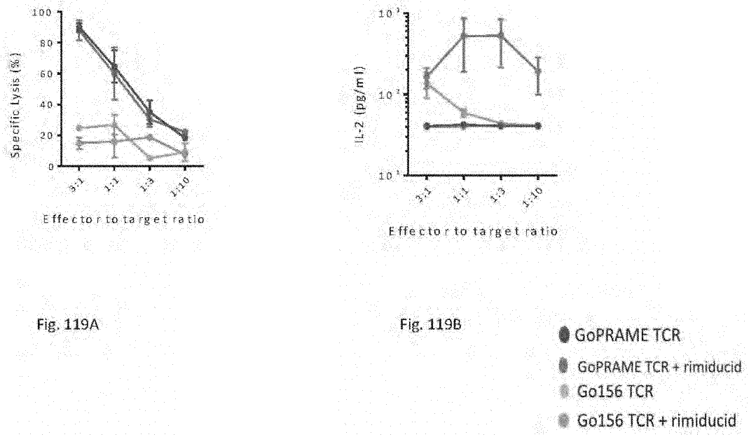

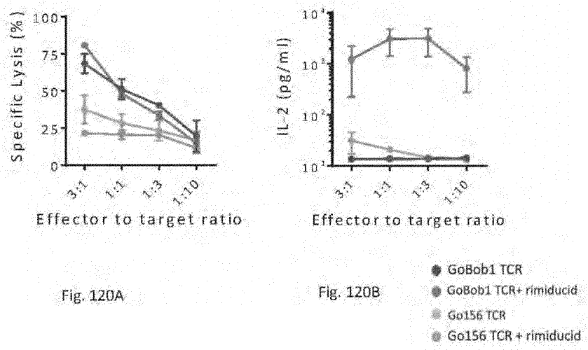

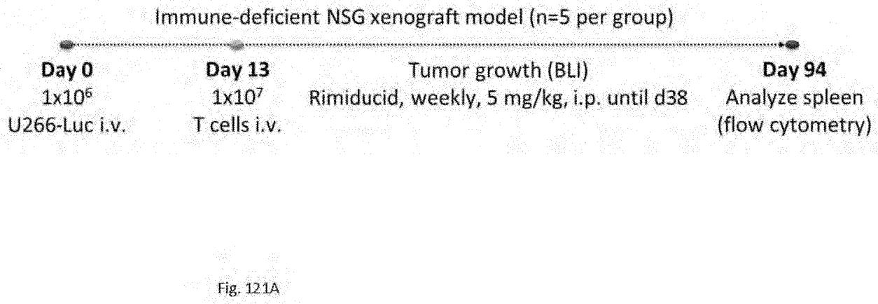

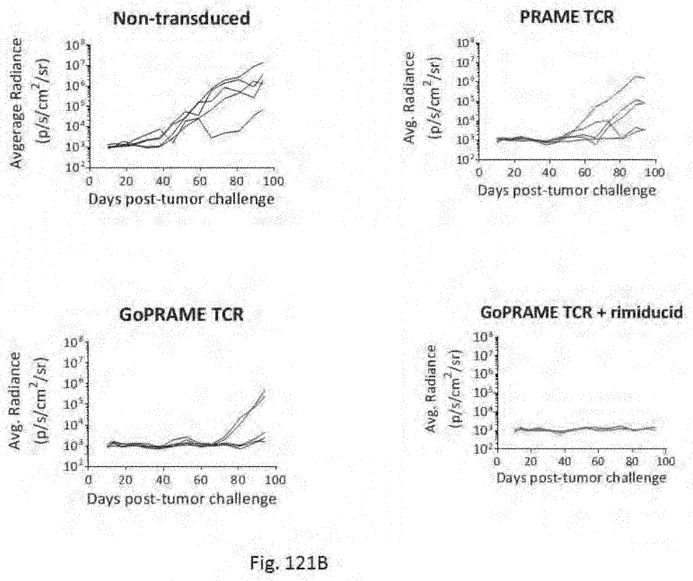

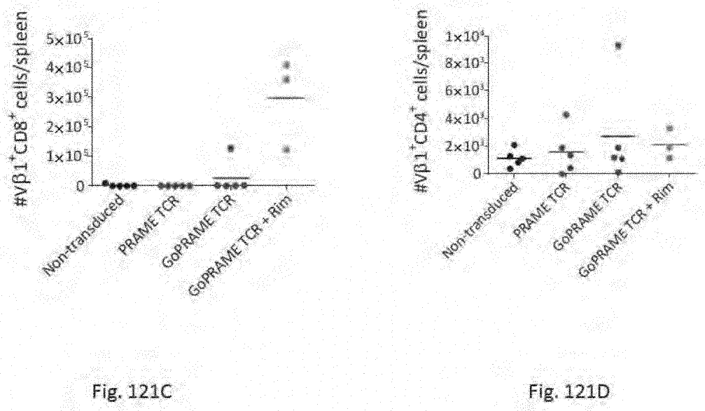

To normalize gated populations, percentages of CD34⁺CD19⁺ cells were divided by percent CD19⁺CD34⁻ cells within each sample as an internal control. Those values were then normalized to drug free wells for each transduction which were set at 100%. Similar analysis was applied to the Hi-, Med-, and Lo-expressing cells within the CD34⁺CD19⁺ gate. FIG. 17B. Representative example of how cells were gated for Hi, Med, and Lo expression. FIG. 17C. Representative scatter plots of final CD34 vs CD19 gates. As rapamycin increased, % CD34⁺CD19⁺ cells decreased, indicating elimination of cells. FIG. 17D and FIG. 17E. T cells from a single donor were transduced with ΔMyr.iMC.2A-CD19 (pBP0606) or FRB_{L}2. Caspase9.2A-Q.8stm.zeta (pBP0668). Cells were plated in IL-2-containing media along with varying amounts of rapamycin for 24 or 48 hrs. Cells were then harvested and analyzed, as above.

FIG. 18 Plasmid map of pBP0044: pSH1-iCaspase9 wt

FIG. 19 Plasmid map of pBP0463-pSH1-Fpk-Fpk′.LS.Fpk″.Fpk′″.LS.HA

FIG. 20 Plasmid map of pBP0725-pSH1-FRBI.FRBI′.LS.FRBI″.FRBI′″

FIG. 21 Plasmid map of pBP0465-pSH1-M-FRBI.FRBI′.LS.HA

FIG. 22 Plasmid map of pBP0721-pSH1-M-FRBI.FRBI′.LS.FRBI″.FRBI′″HA

FIG. 23 Plasmid map of pBP0722-pSH1-Fpk-Fpk′.LS.Fpk″.Fpk′″.LS.HA

FIG. 24 Plasmid map of pBP0220-pSFG-iC9.T2A-ΔCD19

FIG. 25 Plasmid map of pBP0756-pSFG-iC9.T2A-dCD19.P2A-FRBI

FIG. 26 Plasmid map of pBP0755-pSFG-iC9.T2A-dCD19.P2A-FRB12

FIG. 27 Plasmid map of pBP0757-pSFG-iC9.T2A-dCD19.P2A-FRB13

FIG. 28 Plasmid map of pBP0655-pSFG-ΔMyr.FRBI.MC.2A-ΔCD19

FIG. 29 Plasmid map of pBP0498-pSFG-ΔMyriMC.FRB12.P2A-ΔCD19

FIG. 30 Plasmid map of pBP0488-pSFG-αHER2.Q.8stm.CD3zeta.Fpk2

FIG. 31 Plasmid map of pBP0467-pSH1-FRBI′.FRBI.LS.ΔCaspase9

FIG. 32 Plasmid map of pBP0606-pSFG-k-ΔMyr.iMC.2A-ΔCD19

FIG. 33 Plasmid map of pBP0607-pSFG-k-iMC.2A-ΔCD19

FIG. 34 Plasmid map of pBP0668-pSFG-FRBIx2.Caspase9.2A-Q.8stm.CD3zeta

FIG. 35 Plasmid map of pBP0608-pSFG-ΔMyriMC.2A-ΔCD19.Q.8stm.CD3zeta

FIG. 36 Plasmid map of pBP0609: pSFG-iMC.2A-ΔCD19.Q.8stm.CD3zeta

FIG. 37A provides a schematic of rimiducid binding to two copies of a chimeric Caspase-9 polypeptide, each having a FKBP12 multimerizing region.

FIG. 37B provides a schematic of rapamycin binding to two chimeric Caspase-9 polypeptides, one of which has a FKBP12 multimerizing region and the other which has a FRB multimerizing region.

FIG. 37C provides a graph of assay results using these chimeric polypeptides.

FIG. 38A provides a schematic of rapamycin or rapalog binding to two chimeric Caspase-9 polypeptides, one of which has a FKBP12v36 multimerizing region and the other which has a FRB variant (FRB_{L}) multimerizing region. FIG. 38B provides a graph of assay results using this chimeric polypeptide.

FIG. 39A provides a schematic of rimiducid binding to two chimeric Caspase-9 polypeptides, each of which has a FKBP12v36 multimerizing region, and rapamycin binding to only one chimeric Caspase-9 polypeptide having a FKBP12v36 multimerizing region. FIG. 39B provides a graph of assay results comparing the effects of rimiducid and rapamycin.

FIG. 40A provides a schematic of rimiducid binding to two chimeric Caspase-9 polypeptides, each of which has a FKBP12v36 multimerizing region, and rapamycin binding to only one chimeric Caspase-9 polypeptide having a FKBP12v36 multimerizing region in the presence of a FRB multimerization polypeptide. FIG. 40B provides a graph of assay results using these polypeptides, comparing the effects of rimiducid and rapamycin.

FIG. 41 provides a plasmid map of pBP0463.pFRBI.LS.dCasp9.T2A.

FIG. 42 provides a plasmid map of pBP044-pSH1.iCasp9WT.

FIGS. 43A-43C Schematics of FwtFRBC9/MC.FvFv containing iFwtFRBC9 or iFRBFwtC9 (collectively, iRC9). In this version of the rapamycin inducible chimeric pro-apoptotic polypeptide, tandem FKBP.FRB (or FRB.FKBP) domains are fused to Δcaspase-9. Rapamycin or rapalogs can induce: 1) scaffold-induced dimerization of FKBP.FRB.ΔC9 (or FRB.FKBP.ΔC9) via the two FKBP domains fused to MC; 2) direct dimerization of FKBP.FRB.ΔC9 (or FRB.FKBP.ΔC9) to induce multimerization of the engineered caspase-9 fusion proteins.

FIGS. 44A-44C Expression profile of iMC+CARζ-T, i9+CARζ+MC, and FwtFRBC9/MC.FvFv T cells. PBMCs from four different donors were activated and transduced with iMC+CARζ-T (608), i9+CARζ+MC (844), and FwtFRBC9/MC.FvFv (1300)-containing vectors. For a vector schematic see FIG. 48. (A) Five days post-transduction, T cell lysates were subjected to Western blot analysis with antibodies to MyD88, caspase-9, and β-actin (which serves to demonstrate equal protein loading in all lanes). Note that iRC9 migrates the same as the endogenous caspase-9 and the added strength of the band denotes the level of the iRC9. (B) CAR expression were analyzed 4, 7, 12, 21, and 29 days post-transduction with anti-CD34-PE and anti-CD3-PerCPcy5 antibodies. (C) T cell viability from cells growing in culture was assessed 3, 5, 12, 21, and 29 days post-transduction using a Cellometer and AOPI viability dye.

FIGS. 45A-45C Rapamycin induces robust apoptosis activation in FwtFRBC9/MC.FvFv T cells. PBMCs from four different donors were activated and transduced with iMC+CARζ-T (608), i9+CARζ+MC (844), and FwtFRBC9/MC.FvFv (1300)-containing vectors. Five days post-transduction, T cells were seeded onto 96-well plates±rimiducid, ±rapamycin, and in the presence of 2 μM caspase 3/7 green reagent. (A) Plates were placed inside the IncuCyte to monitor green fluorescence over time, reflecting cleaved caspase 3/7 reagent. (B) After 48 hours, cells were stained with anti-CD34-PE (FL2) PI (FL4), and Annexin V-PacBlue (FL9), and cleaved caspase 3/7 was detected in the FL1 channel on a Galios cytometer. (C) Culture supernatant was also collected 48 hours after plating, and IL-2 and IL-6 cytokine production was analyzed by ELISA.

FIGS. 46A-46C Q-LEHD-OPh (SEQ ID NO: 2364) efficiently inhibits caspase activation induced by iC9 and iRC9. PBMCs were activated and transduced with i9+CARζ+MC (844) and FwtFRBC9/MC.FvFv (1300) vectors. Seven days post-transduction, T cells were seeded on 96-well plates (A) with increasing rimiducid/rapamycin concentration, (B) with increasing Q-LEHD-OPh (SEQ ID NO: 2364) concentration, and (C) with 20 nM rimiducid/rapamycin and increasing Q-LEHD-OPh (SEQ ID NO: 2364) concentration. Additionally, 2 μM caspase 3/7 green reagent was added to monitor caspase cleavage by IncuCyte.

FIGS. 47A-47D FRB_{L} and caspase-9 N405Q mutants reduce iRC9 activity. PBMCs were activated and transduced with plasmids 1300, 1308, 1316 and 1317. Five days post-transduction, T cells were seeded onto 96-well plates with 0 (A), 0.8 (B), 4 (C), and 20 nM (D) rapamycin. 2 μM caspase 3/7 green reagent was included to monitor caspase activation over time in the IncuCyte.

FIGS. 48A-48D iRC9 is a potent effector of rapamycin-induced apoptosis. (A) Schematic representation of iMC+CARζ-T, i9+CARζ+MC, iFRBC9 and MC.FvFv, and FwtFRBC9/MC.FvFv constructs. (B-D) Activated T cells were transduced with retrovirus encoding iMC+CARζ-T, i9+CARζ+MC, iFRBC9 and MC.FvFv, or FwtFRBC9/MC.FvFv and treated with no drug, 20 nM rapamycin or 20 nM rimiducid and cultured in the presence of 2.5 μM caspase 3/7 green reagent. The 96-well microplate was placed inside the IncuCyte to monitor activated caspase activity (green fluorescence) for 48 hours.

FIGS. 49A-49D iRC9 quickly and efficiently eliminates CAR-T cells in vivo. (A and B) NSG mice were injected i.v. with 10⁷ iMC+CARζ-T, i9+CARζ+MC, iFRBC9 and MC.FvFv or FwtFRBC9/MC.FvFv T cells co-transduced with GFP-Ffluc per mouse. Bioluminescence of CAR T cells was assessed 18 hours (˜18 h) prior to drug treatment, immediately before drug treatment (0 h) and 4.5 h, 18 h, 27 h, and 45 h post-drug treatment. For mice receiving i9+CARζ+MC T cell injection, 5 mg/kg rimiducid was injected i.p. per mouse. For mice receiving iMC+CARζ-T, (iFRBC9 and MC.FvFv) and FwtFRBC9 MC.FvFv T cells, 10 mg/kg rapamycin was injected i.p. per mouse. At 45 h post-drug treatment, mice were euthanized and (C) blood and (D) spleen were collected for flow cytometry analysis with antibodies to hCD3, hCD34, and mCD45.

FIGS. 50A-50D The on- and off-switches in FwtFRBC9/MC.FvFv are efficiently controlled by rimiducid and rapamycin, respectively. PBMCs from donor 920 were activated and co-transduced with GFP-Ffluc and iMC+CARζ-T (189), i9+CARζ+MC (873), or FwtFRBC9/MC.FvFv (1308)-encoding vectors. Seven days post-transduction, T cells were seeded onto 96-well plates at 1:2 and 1:5 E:T ratios with HPAC-RFP cells in the presence of 0, 2, or 10 nM rimiducid and placed in the IncuCyte to monitor the kinetics of T cell-GFP and HPAC-RFP growth. (A & B) Two days post-seeding, culture supernatants were analyzed for IL-2, IL-6, and IFN-γ production by ELISA. At day 7, 10 nM rimiducid was added to i9+CARζ+MC culture and 10 nM rapamycin was added to GFP, iMC+CARζ-T and FwtFRBC9/MC.FvFv cultures followed by monitoring by IncuCyte until day 8. Numbers of HPAC-RFP and T cell-GFP at the E:T 1:2 ratio was analyzed using the basic analyzer software for the IncuCyte at day 7 (Ci) and day 8 with 0 nM suicide drug (Cii) and 10 nM suicide drug (Ciii). Similar analysis was also performed at the 1:5 E:T ratio (D). (Note: the y-axis in Ci and Di are at log-scale).

FIGS. 51A-51D iRC9 activates apoptosis via direct self-dimerization independent of scaffold-induced dimerization in FwtFRBC9/MC.FvFv. PBMCs from donor 920 were activated and transduced with various vectors de in (A). (B) Protein expression of the CAR T cells was analyzed by Western blot using antibodies to hMyD88, hCaspase-9 and β-actin. (C-D) Five days post-transduction, T cells were seeded on 96-well plates with increasing rapamycin concentrations. Additionally, 2 μM caspase 3/7 green reagent was added to monitor caspase cleavage by IncuCyte. Line graphs depict caspase activation over 24 hours post-rapamycin treatment of MC variants (C) and FRB.FKBP.ΔC9 versus FKBP.FRB.ΔC9 iRC9(D).

FIGS. 52A-52B Relatively high (>100 nM) rimiducid concentration is required to activate iRC9. 293 cells were seeded at 300,000 cells/well in a 6-well plate and allowed to grow for 2 days. After 48 h, cells were transfected with 1 μg of experimental plasmids. Cells were harvested 48 h after transfection and diluted 2.5× their original volume. (A) For the Incucyte/casp3/7 assay, 50 μl of cells were plated per well including either rimiducid or rapamycin drug and caspase 3/7 green reagent (2.5 μM final concentration). (B) For the SEAP assays, 100 μl of cells were plated in a 96-well plate with (half-log) rimiducid (or rapamycin) drug dilutions and ˜18 h after drug exposure, plates were heat-inactivated before substrate (4-MUP) addition.

FIGS. 53A-53B Schematic of MC-Rap, a CAR-costimulation strategy inducible with rapamycin or rapalogs. In this version of an inducible costimulatory switch, tandem FKBP.FRB (or FRB.FKBP) domains are fused to MyD88-CD40 (MC) (right). Rapamycin or rapalogs can induce direct dimerization of FKBP in MC-FKBP-FRB (or MC-FRB-FKBP) with FRB in a second molecule of MC-FKBP-FRB to induce multimerization of the engineered MC fusion proteins. Note that FRB can be present as the wild-type or as a mutant such as FRB_{L} inducible with rapalogs that have reduced affinity for mTOR. This strategy is contrasted with homodimerization directed by rimiducid and FKBP_{V36} in the iMC+CARζ platform (left).

FIGS. 54A-54B Induction of MC costimulatory activity with a rapalog and a MC-Rap-CAR. Human PBMCs were activated and transduced with iMC+CARζ constructs (BP0774 and BP1433), MC-rap-CAR (BP1440) or an noninducible MC only construct (BP1151). Cells were allowed to rest for 6 days then aliquots were stimulated with rimiducid or the rapalog C7-dimethoxy-7-isobutyloxyrapamycin. Supernatant media was harvested 24 hours later and the amount of secreted IL-6 determined by ELISA as an indicator of MC activity. MC activity in iMC+CARζ-T cells is stimulated strongly with rimiducid and not with the rapalog. MC activity in MC-rap-T cells is not stimulated with rimiducid because FKBP12 in pBP1440 is the wild-type rather than the rimiducid sensitive allele V36. MC-Rap activity is instead strongly responsive to isobutyloxyrapamycin to a degree similar to the iMC+CARζ-Ts with rimiducid.

FIGS. 55A-55B Protein expression of MC from iMC+CAR. Human PBMCs were activated and transduced with iMC+CARζ constructs (BP0774, BP1433 and BP1439), MC-rap-CAR (BP1440) or an noninducible MC only constructs (BP1151 oriented at the 5′ end of the retrovirus and 1414 oriented 3′ relative to the CAR). Cells were expanded for 2 weeks then extracts were prepared for SDS-PAGE. Western blots were probed with antibodies to MyD88. The MC-FKBP-FRB fusion protein was expressed at a similar level to the MC-FKBPv fusions from iMC+CARζ constructs.

FIGS. 56A-56B Responsiveness of MC-rap to dosage of rapamycin and rapamycin analog. 293T cells were transfected with 1 μg of reporter construct NF-κB SeAP and 4 μg of the iMC+CARζ construct pBP0774 or the MC-rap-CAR construct pBP1440 using the GeneJuice protocol (Novagen). 24 hours post transfection cells were split to 96 well plates and incubated with increasing concentrations of rimiducid, rapamycin or isobutyloxyrapamycin. After 24 hours of further incubation SeAP activity was determined from cell supernatants. NF-κB reporter activity was stimulated with a subnanomolar EC50 with both the rapalog and rapamycin while up to 50 nM rimiducid could not direct MC-rap dimerization.

FIGS. 57A-57B Schematic of MC-Rap, a CAR-costimulation strategy inducible with rapamycin or rapalogs. In FwtFRBC9/MC.FvFv (left) tandem FKBP.FRB (or FRB.FKBP) domains are fused to Caspase 9 and tandem Fv moieties are fused to MC. Caspase 9 can be activated by homodimerization through rapamycin directed FRB and wild-type FKBP ligation or by scaffolding with iMC. Rimiducid dimerizes FKBP_{V36} moieties to activate MC. FRBFwtMC/FvC9 (right) uses rapamycin or rapalogs can to induce MC-rap while iC9 induced by rimiducid for a cell suicide switch.

FIGS. 58A-58C FRBFwtMC/FvC9 can effectively control tumor growth but is abrogated by activation of iC9 with rimiducid. PBMCs from donor 676 were activated and transduced with a CD19 directed i9+CARζ+MC (BP0844), FRBFwtMC/FvC9 (BP1460) or FwtFRBC9/MC.FvFv (BP1300). Seven days post-transduction, T cells were seeded onto 24-well plates at 1:5 E:T ratios with Raji-GFP cells in the presence of 2 nM rimiducid, 2 nM isobutyloxyrapamycin or 2 nM rapamycin. After seven days of incubation the live cells were analyzed for the proportion of GFP labeled tumor cells (left) and for the proportion of total T cells (CD3⁺, right) and transduced CAR-T cells (CD34, not shown). Rimiducid caused cell death of CAR-T cells with i9+CARζ+MC, or FRBFwtMC/FvC9 and tumor cells dominate the culture while rapamycin or isobutyloxyrapamycin cause cell death with FwtFRBC9/MC.FvFv.

FIG. 59 Schematic of plasmid pBP1300-pSFG-FKBP.FRB.ΔC9.T2A-αCD19.Q.CD8stm.ζ.P2A-iMC

FIG. 60 Schematic of plasmid pBP1308-pSFG-FKBP.FRB.ΔC9.T2A-αPSCA.Q.CD8stm.ζ.P2A-iMC

FIG. 61 Schematic of plasmid pBP1310-pSFG.FRB.FKBP.ΔC9.T2A-ΔCD19

FIG. 62 Schematic of plasmid pBP1311-pSFG.FKBP.FRB.ΔC9.T2A-ΔCD19

FIG. 63 Schematic of plasmid pBP1316-pSFG-FKBP.FRB_{L}.ΔC9.T2A-αPSCA.Q.CD8stm.ζ.P2A-iMC

FIG. 64 Schematic of plasmid pBP1317-pSFG-FKBP.FRB.ΔC9_{Q}.T2A-αPSCA.Q.CD8stm.ζ.P2A-iMC

FIG. 65 Schematic of plasmid pBP1319-pSFG-FKBP.FRB.ΔC9.T2A-αPSCA.Q.CD8stm.ζ.P2A-MC.FKBP_{V}

FIG. 66 Schematic of plasmid pBP1320-pSFG-FKBP.FRB.ΔC9.T2A-αPSCA.Q.CD8stm.ζ.P2A-MC

FIG. 67 Schematic of plasmid pBP1321-pSFG-FKBP.FRB.ΔC9.T2A-αPSCA.Q.CD8stm.ζ.P2A-MC.FKBP_{V}.FKBP

FIG. 68A provides a graph of drug-dependent CAR-T cell killing of tumor cells. FIG. 68B provides schematics of inducible MyD88-CD40 polypeptides.

FIG. 69A provides a schematic representation of retroviral vectors that express inducible MyD88-CD40 polypeptides.

FIG. 69B provides a bar graph of results of a reporter assay of costimulatory signaling.

FIG. 69C provides a bar graph of CAR-T cell cytokine secretion.

FIG. 69D provides a graph of a CAR-T cell killing assay.

FIG. 70A provides a schematic representation of retroviral vectors that express inducible MyD88-CD40 polypeptides.

FIG. 70B provides a graph of a reporter assay of costimulatory signaling.

FIG. 70C provides a graph of a PSCA-CAR-T cell killing assay.

FIG. 70D provides a graph of a PSCA CAR-T cell killing assay.

FIG. 70E provides a graph of a HER2-CAR-T cell killing assay.

FIG. 70F provides a graph of a HER2-CAR-T cell killing assay.

FIG. 70G provides a graph of a HER2-CAR-T cell killing assay.

FIG. 71A provides a graph of apoptosis activity directed by inducible Caspase-9 in the presence of rimiducid.

FIG. 71B provides a graph of apoptosis activity directed by inducible Caspase-9 in the presence of C7-isobutyloxyrapamycin.

FIG. 72A provides a schematic of polypeptides expressed on a single vector, including a CAR polypeptide, a iRC9 polypeptide, and an iMC polypeptide.

FIG. 72B provides schematics of the polypeptides expressed on two separate vectors.

FIG. 73A provides a schematic of inducible Caspase 9 retroviral constructs.

FIG. 73B provides data showing fluorescent conversion of cells that express Caspase 9 in the presence of rapamycin.

FIG. 73C provides a graph of relative apoptosis activity of FIG. 73B.

FIG. 73D provides a Western blot of Caspase-9 transgene expression in T cells.

FIG. 74A provides a graph of IL-6 secretion in the presence of rimiducid.

FIG. 74B provides a graph of IL-2 secretion in the presence of rimiducid.

FIG. 74C provides a graph of IFN-γ secretion in the presence of rimiducid.

FIG. 74D provides a graph of CAR-T cell killing in the presence of rimiducid.

FIG. 74E provides a Western blot of expression of iMC and iRC9.

FIG. 75A provides cell sorting results from non-transduced T cells, or T cells transduced with retroviruses that encode iRC9, iMC, and CAR, as indicated.

FIG. 75B provides a graph of the results of FIG. 75A.

FIG. 75C provides cell sorting results of an apoptosis assay.

FIG. 75D provides a graphical representation of an apoptosis assay.

FIG. 76A provides micrographs of tumor bearing animals determined by bioluminescence imaging.

FIG. 76B provides graphs of average tumor growth.

FIG. 76C provides graphs of human T cells in spleens at termination.

FIG. 76D provides graphs of vector copy number.

FIG. 77A provides micrographs of tumor-bearing animals determined by bioluminescence imaging.

FIG. 77B provides graphs of average radiance.

FIG. 77C provides a graph of a Kaplan-Meier analysis from FIG. 77A.

FIG. 77D provides a representative FACS analysis at termination.

FIG. 78A provides micrographs of tumor-bearing animals determined by bioluminescence imaging.

FIG. 78B provides graphical representations of the average calculated radiance from FIG. 78A.

FIG. 78C provides a graph of human T cell counts in mouse spleens.

FIG. 79A provides micrographs of tumor-bearing animals determined by bioluminescence imaging.

FIG. 79B provides a graphical representation of the average calculated radiance from FIG. 79A.

FIG. 79C provides a graph of the number of human T cells in mouse spleens at termination.

FIG. 79D provides graphs of vector copy number from DNA derived from mouse spleens.

FIG. 80 provides a plasmid map of pBP1151-pSFG-MC-T2A-αCD19.Q.CD8stm.ζ

FIG. 81 provides a plasmid map of pBP1152-pSFG-MC-T2A-αCD19.Q.CD8stm.ζ

FIG. 82 provides a plasmid map of pBP1414-pSFG-αCD19.Q.CD8stm.ζ-P2A-MC

FIG. 83 provides a plasmid map of pBP1414-pSFG-αCD19.Q.CD8stm.ζ-P2A-MC

FIG. 84 provides a plasmid map of pBP1433-pSFG-Fv-Fv-MC-T2A-αCD19.Q.CD8stm.ζ

FIG. 85 provides a plasmid map of pBP1439-pSFG-MC.FKBPᵥ-T2A-αCD19.Q.CD8stm.ζ

FIG. 86 provides a plasmid map of pBP1440-pSFG-FKBPv.ΔC9.T2A-αCD19.Q.CD8stm.ζ.T2A.P2A-MC.FKBP_{wt}.FRB_{L}

FIG. 87 provides a plasmid map of pBP1460-pSFG-FKBPv.ΔC9.T2A-αCD19.Q.CD8stm.ζ.T2A.P2A-MC.FKBP_{wt}.FRB_{L}

FIG. 88 provides a plasmid map of pBP1293-pSFG-iMC.T2A-αhCD33(My9.6).ζ

FIG. 89 provides a plasmid map of pBP1296-pSFG-iMC.T2A-αhCD123(32716).ζ

FIG. 90 provides a plasmid map of pBP1327-pSFG-FRB.FKBP_{V}.ΔC9.2A-ΔCD19

FIG. 91 provides a plasmid map of pBP1328-pSFG-FKBP_{V}.FRB.ΔC9.2A-ΔCD19

FIG. 92 provides a plasmid map of pBP1351-pSFG-SP163.FKBP.FRB.ΔC9.T2A-αhPSCA.Q.CD8stm.ζ.2A-iMC

FIG. 93 provides a plasmid map of pBP1373-pSFG-sp-FKBP.FRB.ΔC9.T2A-αhPSCAscFv.Q.CD8stm.ζ

FIG. 94 provides a plasmid map of pBP1385-pSFG-FRB.FKBP.ΔC9.T2A-ΔCD19

FIG. 95 provides a plasmid map of pBP1455-pSFG-MC.FKBP_{wt}.FRB_{L}.T2A-αPSCA.Q.CD8stm.ζ

FIG. 96 provides a plasmid map of pBP1466-pSFG-FKBPv.ΔC9.T2A-PSCA.Q.CD8stm.ζ.P2A-MC.FKBP_{wt}.FRB_{L}

FIG. 97 provides a plasmid map of pBP1474-pSFG-FKBPv.ΔC9.T2A-αHER2.Q.CD8stm.ζ

FIG. 98 provides a plasmid map of pBP1475-pSFG-FKBPv.ΔC9.T2A-αPSCA.Q.CD8stm.ζ

FIG. 99 provides a plasmid map of pBP1488-pSFG-FRB_{L}.FKBP_{wt}.MC-T2A-αPSCA.Q.CD8stm.ζ

FIG. 100 provides a plasmid map of pBP1491-pSFG-FKBPv.ΔC9.P2A.MC.FKBP_{wt}.FRB_{L}.T2A-αHER2.Q.CD8stm.ζ

FIG. 101 provides a plasmid map of pBP1493-pSFG-MC.FKBP_{wt}.FRB_{L}-P2A.FKBPv.ΔC9.T2A-αHER2.Q.CD8stm.ζ

FIG. 102 provides a plasmid map of pBP1494-pSFG-MC.FKBP_{wt}.FRB_{L}-P2A.FKBPv.ΔC9.T2A-PSCA.Q.CD8stm.ζ

FIG. 103 provides a plasmid map of pBP1757-pSFG-FRB_{L}.FKBP_{wt}.MC-P2A.FKBPv.ΔC9.T2A-αPSCA.Q.CD8stm.ζ

FIG. 104 provides a plasmid map of pBP1759-pSFG-FRB_{L}.FKBP_{wt}.MC-P2A.FKBPv.ΔC9.T2A-αHER2.Q.CD8stm.ζ

FIG. 105 provides a plasmid map of pBP1796-pSFG-FKBP_{wt}.FRB_{L}-MC. P2A.FKBPv.ΔC9.T2A-αPSCA.Q.CD8stm.ζ

FIG. 106A provides a schematic of various inducible chimeric Caspase-9 constructs. FIG. 106B provides graphs of caspase activation assays. FIG. 106C is a photo of a Western blot showing protein expression.

FIG. 107A provides graphs of caspase activity. FIG. 107B provides graphs of SEAP activity.

FIG. 108A provides graphs of SEAP activity. FIG. 108B provides graphs of caspase activity. FIG. 108C provides a Western blot showing protein expression.

FIG. 109A provides a FACS analysis of transduction efficiency. FIG. 109B provides graphs of bioluminescence. FIG. 109C provides photos of bioluminescence in mice. FIG. 109D provides graphs of FACs analysis of mice spleen cells.

FIG. 110A provides a FACs analysis of transduction efficiency. FIG. 110B provides graphs of bioluminescence. FIG. 110C provides photos of bioluminescence in mice. FIG. 110D provides a graph of FACs analysis of mice spleen cells.

FIG. 111 provides a schematic of a vector encoding a CD123-CAR-ζ and an iMC polypeptide.

FIG. 112A provides a graph of IL-6 production; FIG. 112 B provides a graph of IL-2 production;

FIG. 112C provides a graph of total green fluorescence intensity of THP1-GP.Fluc, and FIG. 112D provides a graph of number of HPAC-RFP cells.

FIG. 113A provides a graph of IL-2 production; FIG. 113B provides a graph of THP1-FP.Fluc cells; FIG. 113C provides a graph of T cells-RFP; FIG. 113D provides a graph of THP1-GFP.Fluc green fluorescence; and FIG. 113E provides a graph of T cell-RFP red fluorescence.

FIG. 114A provides a FACs analysis; FIG. 114B provides a schematic of tumor growth via IVIS monitoring; FIG. 114C provides photos of bioluminescence in mice.

FIG. 115A provides photos of bioluminescence in mice; FIG. 115B provides a graph of vector copy number.

FIG. 116 provides a schematic of inducible MC expressed with a recombinant TCR.

FIG. 117A provides a schematic of a PRAME TCR polypeptide; FIG. 117B provides a schematic of an iMC polypeptide; FIG. 117C provides a schematic of a PRAME-TCR polypeptide co-expressed with an iMC polypeptide; FIG. 117D provides a graph of IL-2 production, items listed along the X-axis are in the same order as the legend.

FIG. 118A provides a schematic of trans-well assay set-up; FIG. 118B provides a graph of HLA-A, B, C levels.

FIG. 119 A provides a graph of specific lysis. FIG. 119B provides a graph of IL-2 production.

FIG. 120A provides a graph of specific lysis; FIG. 120 B provides a graph of IL-2 production.

FIG. 121A provides a schematic of an immune-deficient NSG xerograph model; FIG. 121B provides graphs of average radiance in non-transduced and transduced cells; FIG. 121C provides a graph of the number of Vβ1⁺CD8⁺ cells/spleen; FIG. 121D provides a graph of the number of Vβ1⁺CD8⁺ cells/spleen.

### DETAILED DESCRIPTION

As a mechanism to translate information from the external environment to the inside of the cell, regulated protein-protein interactions evolved to control most, if not all, signaling pathways. Transduction of signals is governed by enzymatic processes, such as amino acid side chain phosphorylation, acetylation, or proteolytic cleavage that lack intrinsic specificity. Furthermore, many proteins or factors are present at cellular concentrations or at subcellular locations that preclude spontaneous generation of a sufficient substrate/product relationship to activate or propagate signaling. An important component of activated signaling is the recruitment of these components to signaling “nodes” or spatial signaling centers that efficiently transmit (or attenuate) the pathway via appropriate upstream signals.

As a tool to artificially isolate and manipulate individual protein-protein interactions and hence individual signaling proteins, chemically induced dimerization (CID) technology was developed to impose homotypic or heterotypic interactions on target proteins to reproduce natural biological regulation. In its simplest form, a single protein would be modified to contain one or more structurally identical ligand binding domains, which would then be the basis of homodimerization or oligomerization, respectively, in the presence of a cognate homodimeric ligand (Spencer D M et al (93) Science 262, 1019-24). A slightly more complicated version of this concept would involve placing one or more distinct ligand binding domains on two different proteins to enable heterodimerization of these signaling molecules using small molecule, heterodimeric ligands that bind to both distinct domains simultaneously (Ho S N et al (96) Nature 382, 822-6). This drug-mediated dimerization creates a very high local concentration of ligand binding-domain-tagged components sufficient to permit their induced or spontaneous assembly and regulation.

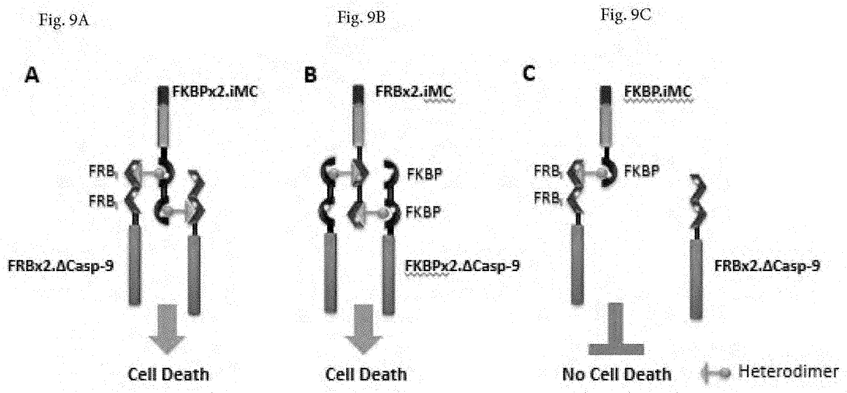

In some embodiments, provided herein are methods to induce multimerization of proteins. In this case, two or more heterodimer ligand binding regions (or “domains”) in tandem are used as a “molecular scaffold” to dimerize or oligomerize a second, signaling domain-containing protein that is fused to one or more copies of the second binding site for the heterodimeric ligand. The molecular scaffold can be expressed as an isolated multimer of ligand binding domains (FIG. 8), either localized within the cell or unlocalized (FIG. 8B, 8C), or it can be attached to another protein that provides a structural, signaling, cell marking, or more complex combinatorial function (FIG. 9). By “scaffold” is meant a polypeptide that comprises at least two, for example, two or more, heterodimer ligand binding regions; in certain examples the ligand binding regions are in tandem, that is, each ligand binding region is located directly proximal to the next ligand binding region. In other examples, each ligand binding region may be located close to the next ligand binding region, for example, separated by about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80 or more amino acids, but retain the scaffold function of dimerization of an inducible caspase molecule in the presence of a dimerizer. A scaffold may comprise, for example, at least 2, 3, 4, 5, 6, 7, 8, 9, 10, 11, 12, 13, 14, 15, 16, 17, 18, 19, 20 or more ligand binding regions, and may also be linked to another polypeptide, such as, for example, a marker polypeptide, a costimulating molecule, a chimeric antigen receptor, a T cell receptor, or the like.

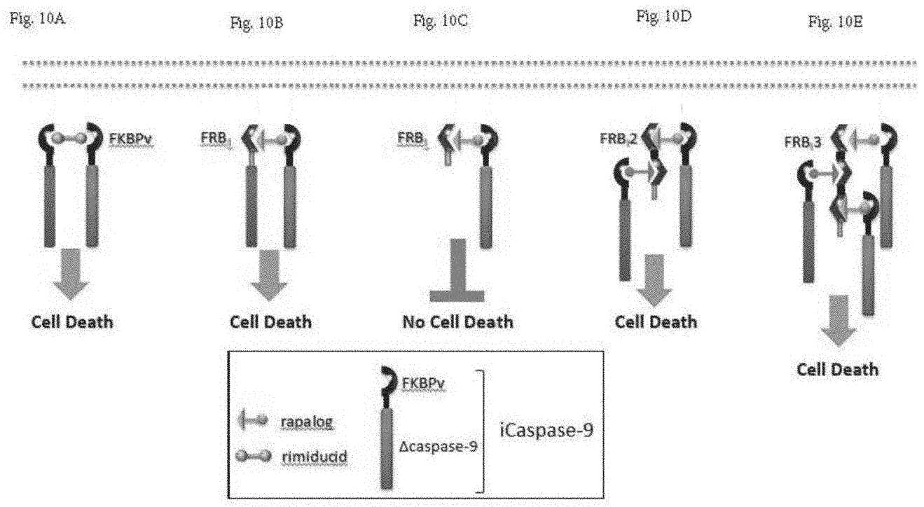

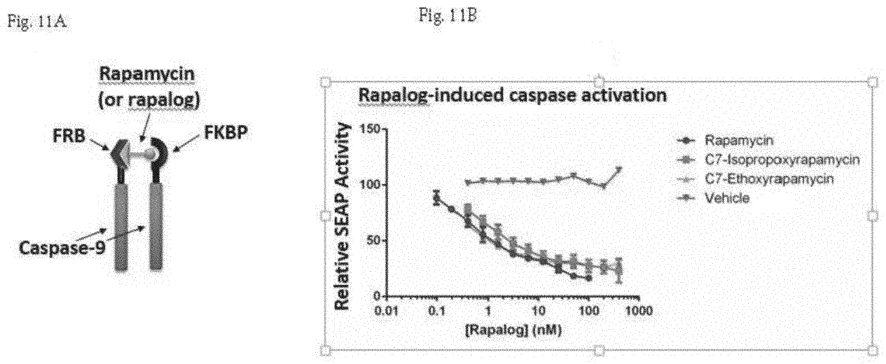

In some embodiments, the first polypeptide consists essentially of at least two, three, four, five, six, seven, eight, nine, ten, 11, 12, 13, 14, 15, 16, 17, 18, 19, or 20 units of the first multimerizing region. In some embodiments, first polypeptide consists essentially of the scaffold region. In some embodiments, the first polypeptide consists essentially of a membrane association region or a membrane targeting region. By “consists essentially of” is meant that the scaffold units or the scaffold may be alone, can optionally include linker polypeptides at either terminus of the scaffold, or between the units, and can optionally include small polypeptides such as, for example stem polypeptides as shown in FIGS. 10B, 10C, 10D, and 10E.

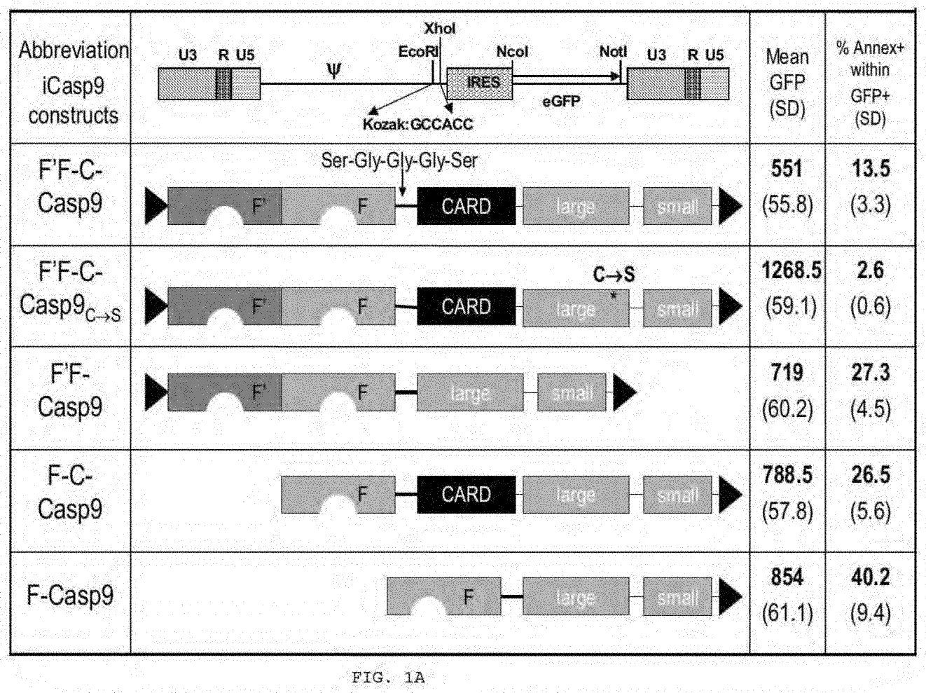

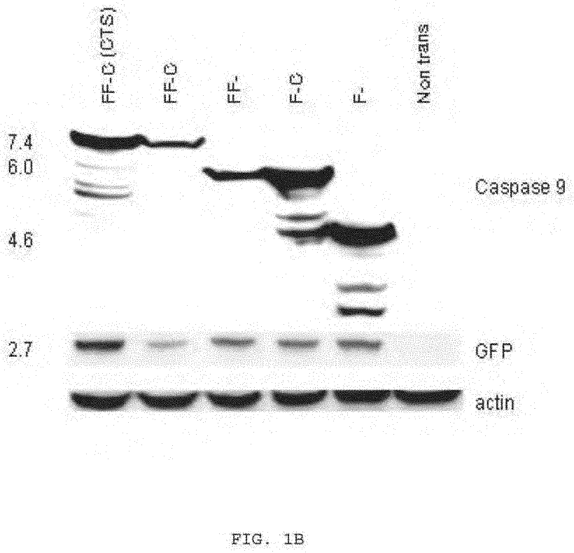

In one example, a tandem multimer of the ˜89 aa FK506-rapamycin binding (FRB) domain derived from the protein kinase mTOR (Chen J et al (95) PNAS, 92, 4947-51) is used to recruit multiple FKBPv36-fused Caspase-9 (iC9/iCaspase-9) in the presence of rapamycin or a rapamycin-based analogue (“rapalog”) (Liberles S D (97) PNAS 94, 7825-30; Rivera V M (96) Nat Med 2, 1028-1032, Stankunas K (03) Mol Cell 12, 1615-24; Bayle J H (06) Chem & Biol, 13, 99-107) (FIGS. 1-3). This recruitment leads to spontaneous caspase dimerization and activation.

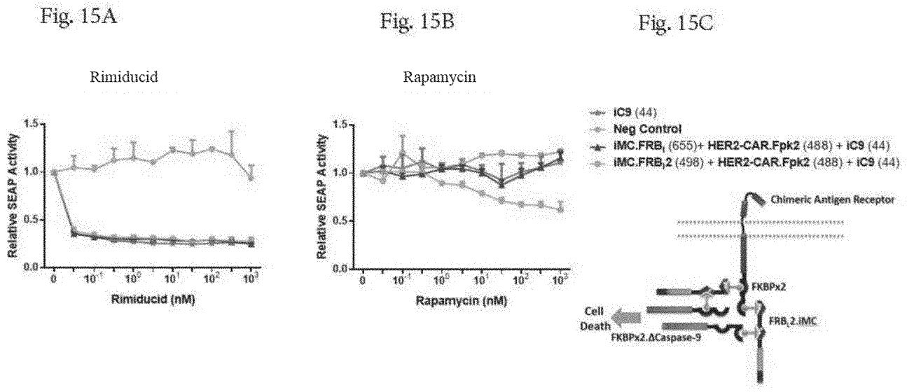

In a second example, the tandem FRB domains are fused to a chimeric antigen receptor (CAR) and this provides rapalog-driven iC9 activation to cells expressing both fusion proteins (FIG. 15, inset).

In a third example, the polarity of the two proteins are reversed so that two or more copies of FKBP12 are used to recruit and multimerize FRB-modified signaling molecules in the presence of rapamycin (FIG. 8C, 9A).

In some examples, a chimeric polypeptide may comprise a single ligand binding region, or a scaffold comprising more than one ligand binding region may be, where the chimeric polypeptide comprises a polypeptide such as, for example, a MyD88 polypeptide, a truncated MyD88 polypeptide, a cytoplasmic CD40 polypeptide, a chimeric MyD88/cytoplasmic CD40 polypeptide or a chimeric truncated MyD88/cytoplasmic CD40 polypeptide.

By MyD88, or MyD88 polypeptide, is meant the polypeptide product of the myeloid differentiation primary response gene 88, for example, but not limited to the human version, cited as ncbi Gene ID 4615. By “truncated,” is meant that the protein is not full length and may lack, for example, a domain. For example, a truncated MyD88 is not full length and may, for example, be missing the TIR domain. An example of a truncated MyD88 polypeptide amino acid sequence is presented as SEQ ID NO: 969. By a nucleic acid sequence coding for “truncated MyD88” is meant the nucleic acid sequence coding for the truncated MyD88 peptide, the term may also refer to the nucleic acid sequence including the portion coding for any amino acids added as an artifact of cloning, including any amino acids coded for by the linkers. It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide.

In the methods herein, the CD40 portion of the peptide may be located either upstream or downstream from the MyD88 or truncated MyD88 polypeptide portion.

In a fourth example, unstable FRB variants (e.g., FRBL2098) are used to destabilize the signaling molecule prior to rapalog administration (Stankunas K (03) Mol Cell 12, 1615-24; Stankunas K (07) ChemBioChem 8, 1162-69) (FIG. 9, 10). Following rapalog exposure, the unstable fusion molecule is stabilized leading to aggregation as before, but with lower background signaling.

The use of ligands to direct signaling proteins may be generally applied to activate or attenuate many signaling pathways. Examples are provided herein that demonstrate a utility of the approach by controlling apoptosis or programmed cell death with the “initiating caspase”, Caspase-9 as the primary target. Control of apoptosis by dimerization of proapoptotic proteins with widely available rapamycin or more proprietary rapalogs, should permit an experimenter or clinician to tightly and rapidly control the viability of a cell-based implant that displays unwanted effects. Examples of these effects include, but are not limited to, Graft versus Host (GvH) immune responses against off-target tissue or excessive, uncontrolled growth or metastasis of an implant. Rapid induction of apoptosis will severely attenuate the unwanted cell's function and permit the natural clearance of the dead cells by phagocytic cells, such as macrophages, without undue inflammation.

Apoptosis is tightly regulated and naturally uses scaffolds, such as Apaf-1, CRADD/RAIDD, or FADD/Mort1, to oligomerize and activate the caspases that can ultimately kill the cell. Apaf-1 can assemble the apoptotic protease Caspase-9 into a latent complex that then forms an active oligomeric apoptosome upon recruitment of cytochrome C to the scaffold. The key event is oligomerization of the scaffold units causing dimerization and activation of the caspase. Similar adapters, such as CRADD, can oligomerize Caspase-2, leading to apoptosis. The compositions and methods provided herein use, for example, multimeric versions of the ligand binding domains FRB or FKBP to serve as scaffolds that permit the spontaneous dimerization and activation of caspase units present as FRB or FKBP fusions upon recruitment with rapamycin.

Using certain of the methods provided in the examples herein, caspase activation occurs only when rapamycin or rapalogs are present to recruit the FRB or FKBP-fused caspase to the scaffold. In these methods, the FRB or FKBP polypeptides must be present as a multimeric unit not as monomers to drive FKBP- or FRB-caspase dimerization (except when FRB-Caspase-9 is dimerized with FKBP-Caspase-9). The FRB or FKBP-based scaffold can be expressed in a targeted cell as a fusion with other proteins and retains its capacity to serve as a scaffold to assemble and activate proapoptotic molecules. The FRB or FKBP scaffold may be localized within the cytosol as a soluble entity or present in specific subcellular locales, such as the plasma membrane through targeting signals. The components used to activate apoptosis and the downstream components that degrade the cell are shared by all cells and across species. With regard to Caspase-9 activation, these methods can be broadly utilized in cell lines, in normal primary cells, such as, for example, but not limited to, T cells, or in cell implants.

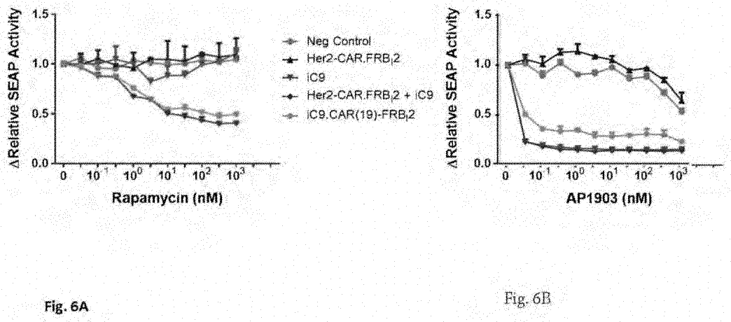

In certain examples of the direct dimerization of FRB-Caspase with FKBP-Caspase with rapamycin to direct apoptosis, it was shown that FKBP-fused Caspases can be dimerized by homodimerizer molecules, such as AP1510, AP20187 or AP1903 (FIG. 6 (right panel), 10A (schematic) (A similar proapoptotic switch can be directed via heterodimerization of a binary switch using rapamycin or rapalogs by coexpression of a FRB-Caspase-9 fusion protein along with FKBP-Caspase-9, leading to homodimerization of the caspase domains within the chimeric proteins (FIG. 8A (schematic), 10B (schematic), (11).

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The following table outlines the nature of some of the nomenclature and acronyms for the switches discussed in this and the following examples.

| Short Name | Molecular Construct | Other Reference |
|---|---|---|
| iC9, FvC9, iCasp-9, | FKBPvΔC9 | FKBP12v36-Caspase-9, |
| iCaspase-9 | | CaspaCIDe |
| FRB.C9, FRB.Casp-9 | FRBΔC9 | RapaCIDe-1.0 |
| iC9 + FRB.C9 | FKBP12ΔC9 + FRBΔC9 | RapaCIDe-2.0 |
| iRC9, FwtFRB.C9 | FKBP.FRBΔC9 | FKBP12-FRBΔC9, |
| | | RapaCIDe-3.0, FFC9, iFFC9 |
| iRC9, FRB.FwtC9 | FRB.FKBPΔC9 | FRB-FKBP12ΔC9, |
| | | RapaCIDe-3.1, FFC9, iFFC9 |
| iMC, MC.FvFv | MC.FKBPv.FKBPv | MC. FKBP12v36- |
| | | FKBP12v36, inducible |
| | | MyD88/CD40, FvFvMC |
| | | (variant), FFMC, iFFMC |
| iRMC, FRB.FwtMC | FRB.FKBPwtMC or | FRBFwtMC or FwtFRBMC, |
| | FKBPwt.FRBMC | MC-Rap |
| iRMC, MC.FRB.Fwt | MC.FRB.FKBPwt or | MC.FRBFwt or MC.FwtFRB, |
| | MC.FKBPwt.FRB | MC-Rap |
| iC9 + CARζ + iRMC | FvΔC9 + CARζ + | DragCAR-3.0, variant domain |
| | FRB.FwtMC | permutations |
| iC9 + CARζ + MC | FvΔC9 + CARζ-2A-MC | CIDeCAR |
| iMC + CARζ | MC.FvFv + CARζ | GoCAR |
| iRmC9, FvFRB.C9 | FKBPv.FRBΔC9 | Dual-switch inducible |
| | | caspase, FKBP12v36FRBΔC9, |
| | | RipaCIDe |
| iRmC9, FRB.FvC9 | FRB.FKBPvΔC9 | Dual-switch inducible |
| | | caspase, FRB.FKBP12v36ΔC9, |
| | | RipaCIDe |
| FRB.C9 + iMC + CARζ | FRBΔC9 + MC.FvFv + CARζ | DragCAR-1.0 |
| iRC9 + iMC + CARζ | Fwt.FRBΔC9 + MC.FvFv | DragCAR-2.0 + variant |
| | | domain permutations |

The term “allogeneic” as used herein, refers to HLA or MHC loci that are antigenically distinct.

Thus, cells or tissue transferred from the same species can be antigenically distinct. Syngeneic mice can differ at one or more loci (congenics) and allogeneic mice can have the same background.

The term “antigen” as used herein is defined as a molecule that provokes an immune response. This immune response may involve either antibody production, or the activation of specific immunologically-competent cells, or both.

An “antigen recognition moiety” may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single-chain variable fragments (scFv), Fab, Fab′, F(ab′)2, and Fv fragments; polypeptides derived from T Cell receptors, such as, for example, TCR variable domains; and any ligand or receptor fragment that binds to the extracellular cognate protein.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

Donor: The term “donor” refers to a mammal, for example, a human, that is not the patient recipient. The donor may, for example, have HLA identity with the recipient, or may have partial or greater HLA disparity with the recipient.

Haploidentical: The term “haploidentical” as used with reference to cells, cell types and/or cell lineages, herein refers to cells sharing a haplotype or cells having substantially the same alleles at a set of closely linked genes on one chromosome. A haploidentical donor does not have complete HLA identity with the recipient, there is a partial HLA disparity.

Blood disease: The terms “blood disease”, “blood disease” and/or “diseases of the blood” as used herein, refers to conditions that affect the production of blood and its components, including but not limited to, blood cells, hemoglobin, blood proteins, the mechanism of coagulation, production of blood, production of blood proteins, the like and combinations thereof. Non-limiting examples of blood diseases include anemias, leukemias, lymphomas, hematological neoplasms, albuminemias, haemophilias and the like.

Bone marrow disease: The term “bone marrow disease” as used herein, refers to conditions leading to a decrease in the production of blood cells and blood platelets. In some bone marrow diseases, normal bone marrow architecture can be displaced by infections (e.g., tuberculosis) or malignancies, which in turn can lead to the decrease in production of blood cells and blood platelets. Non-limiting examples of bone marrow diseases include leukemias, bacterial infections (e.g., tuberculosis), radiation sickness or poisoning, apnocytopenia, anemia, multiple myeloma and the like.

T cells and Activated T cells (include that this means CD3⁺ cells): T cells (also referred to as T lymphocytes) belong to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant presented in the context of a Class II major histo-compatibility (MHC) marker. T-cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T-cells (e.g., cells positive for CD3 or CD 4 expression are referred to as CD3⁺ or CD4⁺). CD3 and CD4 proteins are cell surface receptors or co-receptors that may be directly and/or indirectly involved in signal transduction in T cells.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

By “cytoplasmic CD40” or “CD40 lacking the CD40 extracellular domain” is meant a CD40 polypeptide that lacks the CD40 extracellular domain. In some examples, the terms also refer to a CD40 polypeptide that lacks both the CD40 extracellular domain and a portion of, or all of, the CD40 transmembrane domain.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

By “kill” or “killing” as in a percent of cells killed, is meant the death of a cell through apoptosis, as measured using any method known for measuring apoptosis, and, for example, using the assays discussed herein, such as, for example the SEAP assays or T cell assays discussed herein. The term may also refer to cell ablation.

Allodepletion: The term “allodepletion” as used herein, refers to the selective depletion of alloreactive T cells. The term “alloreactive T cells” as used herein, refers to T cells activated to produce an immune response in reaction to exposure to foreign cells, such as, for example, in a transplanted allograft. The selective depletion generally involves targeting various cell surface expressed markers or proteins, (e.g., sometimes cluster of differentiation proteins (CD proteins), CD19, or the like), for removal using immunomagnets, immunotoxins, flow sorting, induction of apoptosis, photodepletion techniques, the like or combinations thereof. In the present methods, the cells may be transduced or transfected with the chimeric protein-encoding vector before or after allodepletion. Also, the cells may be transduced or transfected with the chimeric protein-encoding vector without an allodepletion step, and the non-allodepleted cells may be administered to the patient. Because of the added “safety switch” it is, for example, possible to administer the non-allo-depleted (or only partially allo-depleted) T cells because an adverse event such as, for example, graft versus host disease, may be alleviated upon the administration of the multimeric ligand.

Graft versus host disease: The terms “graft versus host disease” or “GvHD”, refer to a complication often associated with allogeneic bone marrow transplantation and sometimes associated with transfusions of un-irradiated blood to immunocompromised patients. Graft versus host disease sometimes can occur when functional immune cells in the transplanted marrow recognize the recipient as “foreign” and mount an immunologic response. GvHD can be divided into an acute form and a chronic form. Acute GVHD (aGVHD) often is observed within the first 100 days following transplant or transfusion and can affect the liver, skin, mucosa, immune system (e.g., the hematopoietic system, bone marrow, thymus, and the like), lungs and gastrointestinal tract. Chronic GVHD (cGVHD) often begins 100 days or later post transplant or transfusion and can attack the same organs as acute GvHD, but also can affect connective tissue and exocrine glands. Acute GvHD of the skin can result in a diffuse maculopapular rash, sometimes in a lacy pattern.

Donor T cell: The term “donor T cell” as used here refers to T cells that often are administered to a recipient to confer anti-viral and/or anti-tumor immunity following allogeneic stem cell transplantation. Donor T cells often are utilized to inhibit marrow graft rejection and increase the success of alloengraftment, however the same donor T cells can cause an alloaggressive response against host antigens, which in turn can result in graft versus host disease (GVHD). Certain activated donor T cells can cause a higher or lower GvHD response than other activated T cells. Donor T cells may also be reactive against recipient tumor cells, causing a beneficial graft vs. tumor effect.

Mesenchymal stromal cell: The terms “mesenchymal stromal cell” or “bone marrow derived mesenchymal stromal cell” as used herein, refer to multipotent stem cells that can differentiate ex vivo, in vitro and in vivo into adipocytes, osteoblasts and chondroblasts, and may be further defined as a fraction of mononuclear bone marrow cells that adhere to plastic culture dishes in standard culture conditions, are negative for hematopoietic lineage markers and are positive for CD73, CD90 and CD105.

Embryonic stem cell: The term “embryonic stem cell” as used herein, refers to pluripotent stem cells derived from the inner cell mass of the blastocyst, an early-stage embryo of between 50 to 150 cells. Embryonic stem cells are characterized by their ability to renew themselves indefinitely and by their ability to differentiate into derivatives of all three primary germ layers, ectoderm, endoderm and mesoderm. Pluripotent is distinguished from multipotent in that pluripotent cells can generate all cell types, while multipotent cells (e.g., adult stem cells) can only produce a limited number of cell types.

Inducible pluripotent stem cell: The terms “inducible pluripotent stem cell” or “induced pluripotent stem cell” as used herein refers to adult, or differentiated cells, that are “reprogrammed” or induced by genetic (e.g., expression of genes that in turn activates pluripotency), biological (e.g., treatment viruses or retroviruses) and/or chemical (e.g., small molecules, peptides and the like) manipulation to generate cells that are capable of differentiating into many if not all cell types, like embryonic stem cells. Inducible pluripotent stem cells are distinguished from embryonic stem cells in that they achieve an intermediate or terminally differentiated state (e.g., skin cells, bone cells, fibroblasts, and the like) and then are induced to dedifferentiate, thereby regaining some or all of the ability to generate multipotent or pluripotent cells.

CD34⁺ cell: The term “CD34⁺ cell” as used herein refers to a cell expressing the CD34 protein on its cell surface. “CD34” as used herein refers to a cell surface glycoprotein (e.g., sialomucin protein) that often acts as a cell-cell adhesion factor and is involved in T cell entrance into lymph nodes, and is a member of the “cluster of differentiation” gene family. CD34 also may mediate the attachment of stem cells to bone marrow, extracellular matrix or directly to stromal cells. CD34⁺ cells often are found in the umbilical cord and bone marrow as hematopoietic cells, a subset of mesenchymal stem cells, endothelial progenitor cells, endothelial cells of blood vessels but not lymphatics (except pleural lymphatics), mast cells, a sub-population of dendritic cells (which are factor XIIIa negative) in the interstitium and around the adnexa of dermis of skin, as well as cells in certain soft tissue tumors (e.g., alveolar soft part sarcoma, pre-B acute lymphoblastic leukemia (Pre-B-ALL), acute myelogenous leukemia (AML), AML-M7, dermatofibrosarcoma protuberans, gastrointestinal stromal tumors, giant cell fibroblastoma, granulocytic sarcoma, Kaposi's sarcoma, liposarcoma, malignant fibrous histiocytoma, malignant peripheral nerve sheath tumors, mengingeal hemangiopericytomas, meningiomas, neurofibromas, schwannomas, and papillary thyroid carcinoma).

Gene expression vector: The terms “gene expression vector”, “nucleic acid expression vector”, or “expression vector” as used herein, which can be used interchangeably throughout the document, generally refers to a nucleic acid molecule (e.g., a plasmid, phage, autonomously replicating sequence (ARS), artificial chromosome, yeast artificial chromosome (e.g., YAC)) that can be replicated in a host cell and be utilized to introduce a gene or genes into a host cell. The genes introduced on the expression vector can be endogenous genes (e.g., a gene normally found in the host cell or organism) or heterologous genes (e.g., genes not normally found in the genome or on extra-chromosomal nucleic acids of the host cell or organism). The genes introduced into a cell by an expression vector can be native genes or genes that have been modified or engineered. The gene expression vector also can be engineered to contain 5′ and 3′ untranslated regulatory sequences that sometimes can function as enhancer sequences, promoter regions and/or terminator sequences that can facilitate or enhance efficient transcription of the gene or genes carried on the expression vector. A gene expression vector sometimes also is engineered for replication and/or expression functionality (e.g., transcription and translation) in a particular cell type, cell location, or tissue type. Expression vectors sometimes include a selectable marker for maintenance of the vector in the host or recipient cell.

Developmentally regulated promoter: The term “developmentally regulated promoter” as used herein refers to a promoter that acts as the initial binding site for RNA polymerase to transcribe a gene which is expressed under certain conditions that are controlled, initiated by or influenced by a developmental program or pathway. Developmentally regulated promoters often have additional control regions at or near the promoter region for binding activators or repressors of transcription that can influence transcription of a gene that is part of a development program or pathway. Developmentally regulated promoters sometimes are involved in transcribing genes whose gene products influence the developmental differentiation of cells.

Developmentally differentiated cells: The term “developmentally differentiated cells”, as used herein refers to cells that have undergone a process, often involving expression of specific developmentally regulated genes, by which the cell evolves from a less specialized form to a more specialized form in order to perform a specific function. Non-limiting examples of developmentally differentiated cells are liver cells, lung cells, skin cells, nerve cells, blood cells, and the like. Changes in developmental differentiation generally involve changes in gene expression (e.g., changes in patterns of gene expression), genetic re-organization (e.g., remodeling or chromatin to hide or expose genes that will be silenced or expressed, respectively), and occasionally involve changes in DNA sequences (e.g., immune diversity differentiation). Cellular differentiation during development can be understood as the result of a gene regulatory network. A regulatory gene and its cis-regulatory modules are nodes in a gene regulatory network that receive input (e.g., protein expressed upstream in a development pathway or program) and create output elsewhere in the network (e.g., the expressed gene product acts on other genes downstream in the developmental pathway or program).

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used here, the term “rapalog” is meant as an analog of the natural antibiotic rapamycin. Certain rapalogs in the present embodiments have properties such as stability in serum, a poor affinity to wildtype FRB (and hence the parent protein, mTOR, leading to reduction or elimination of immunosuppressive properties), and a relatively high affinity to a mutant FRB domain. For commercial purposes, in certain embodiments, the rapalogs have useful scaling and production properties. Examples of rapalogs include, but are not limited to, S-o,p-dimethoxyphenyl (DMOP)-rapamycin: EC₅₀ (wt FRB (K2095 T2098 W2101)˜1000 nM), EC₅₀ (FRB-KLW˜5 nM) Luengo J I (95) Chem & Biol 2:471-81; Luengo J I (94) J. Org Chem 59:6512-6513; U.S. Pat. No. 6,187,757; R-Isopropoxyrapamycin: EC₅₀ (wt FRB (K2095 T2098 W2101)˜300 nM), EC50 (FRB-PLF˜8.5 nM); Liberles S (97) PNAS 94: 7825-30; and S-Butanesulfonamidorap (AP23050): EC₅₀ (wt FRB (K2095 T2098 W2101)˜2.7 nM), EC₅₀ (FRB-KTF˜>200 nM) Bayle (06) Chem & Bio. 13: 99-107.

The term “FRB” refers to the FKBP12-Rapamycin-Binding (FRB) domain (residues 2015-2114 encoded within mTOR), and analogs thereof. In certain embodiments, FRB analogs or variants are provided. The properties of an FRB analog or variant are stability (some variants are more labile than others) and ability to bind to various rapalogs. In certain embodiments, the FRB analog or variant binds to a C7 rapalog, such as, for example, those provided in the present application, and those referred to in publications that are incorporated by reference herein. In certain embodiments, the FRB analog or variant comprises an amino acid substitution at position T2098. Based on the crystal structure conjugated to rapamycin, there are 3 key rapamycin-interacting residues that have been most analyzed, K2095, T2098, and W2101. Mutation of all three leads to an unstable protein that can be stabilized in the presence of rapamycin or some rapalogs. This feature can be used to further increase the signal:noise ratio in some applications. Examples of mutants are discussed in Bayle et al (06) Chem & Bio 13: 99-107; Stankunas et al (07) Chembiochem 8:1162-1169; and Liberles S (97) PNAS 94:7825-30). Examples of FRB variant polypeptide regions of the present embodiments include, but are not limited to, KLW (with L2098); KTF (with F2101); and KLF (L2098, F2101). FRB variant KLW corresponds to the FRB_{L} polypeptide, for example, consisting of the amino acid of SEQ ID NO: 1085, and has a substitution of an L residue at position 2098. By comparing the KLW variant of SEQ ID NO: 1085 with the wild type FRB polypeptide, for example, the polypeptide consisting of the amino acid sequence of SEQ ID NO: 1066, one can determine the sequence of the other FRB variants listed herein.

Each ligand can include two or more portions (e.g., defined portions, distinct portions), and sometimes includes two, three, four, five, six, seven, eight, nine, ten, or more portions. The first ligand and second ligand each, independently, can consist of two portions (i.e., dimer), consist of three portions (i.e., trimer) or consist of four portions (i.e., tetramer). The first ligand sometimes includes a first portion and a second portion and the second ligand sometimes includes a third portion and a fourth portion. The first portion and the second portion often are different (i.e., heterogeneous (e.g., heterodimer)), the first portion and the third portion sometimes are different and sometimes are the same, and the third portion and the fourth portion often are the same (i.e., homogeneous (e.g., homodimer)). Portions that are different sometimes have a different function (e.g., bind to the first multimerizing region, bind to the second multimerizing region, do not significantly bind to the first multimerizing region, do not significantly bind to the second multimerizing region (e.g., the first portion binds to the first multimerizing region but does not significantly bind to the second multimerizing region) and sometimes have a different chemical structure. Portions that are different sometimes have a different chemical structure but can bind to the same multimerizing region (e.g., the second portion and the third portion can bind to the second multimerizing region but can have different structures). The first portion sometimes binds to the first multimerizing region and sometimes does not bind significantly to the second multimerizing region. Each portion sometimes is referred to as a “monomer” (e.g., first monomer, second monomer, third monomer and fourth monomer that tracks the first portion, second portion, third portion and fourth portion, respectively). Each portion sometimes is referred to as a “side.” Sides of a ligand may sometimes be adjacent to each other, and may sometimes be located at opposing locations on a ligand.

By being “capable of binding”, as in the example of a multimeric or heterodimeric ligand binding to a multimerizing region or ligand binding region is meant that the ligand binds to the ligand binding region, for example, a portion, or portions, of the ligand bind to the multimerizing region, and that this binding may be detected by an assay method including, but not limited to, a biological assay, a chemical assay, or physical means of detection such as, for example, x-ray crystallography. In addition, where a ligand is considered to “not significantly bind” is meant that there may be minor detection of binding of a ligand to the ligand binding region, but that this amount of binding, or the stability of binding is not significantly detectable, and, when occurring in the cells of the present embodiment, does not activate the modified cell or cause apoptosis.

In certain examples, where the ligand does not “significantly bind,” upon administration of the ligand, the amount of cells undergoing apoptosis is less than 10, 5, 4, 3, 2, or 1%.

By “region” or “domain” is meant a polypeptide, or fragment thereof, that maintains the function of the polypeptide as it relates to the chimeric polypeptides of the present application. That is, for example, an FKBP12 binding domain, FKBP12 domain, FKBP12 region, FKBP12 multimerizing region, and the like, refer to an FKBP12 polypeptide that binds to the CID ligand, such as, for example, rimiducid, or rapamycin, to cause, or allow for, dimerization or multimerization of the chimeric polypeptide. By “region” or “domain” of a pro-apoptotic polypeptide, for example, the Caspase-9 polypeptides or truncated Caspase-9 polypeptides of the present applications, is meant that upon dimerization or multimerization of the Caspase-9 region as part of the chimeric polypeptide, or chimeric pro-apoptotic polypeptide, the dimerized or multimerized chimeric polypeptide can participate in the caspase cascade, allowing for, or causing, apoptosis.

As used herein, the term “iCaspase-9” molecule, polypeptide, or protein is defined as an inducible Caspase-9. The term “iCaspase-9” embraces iCaspase-9 nucleic acids, iCaspase-9 polypeptides and/or iCaspase-9 expression vectors. The term also encompasses either the natural iCaspase-9 nucleotide or amino acid sequence, or a truncated sequence that is lacking the CARD domain.

As used herein, the term “iCaspase 1 molecule”, “iCaspase 3 molecule”, or “iCaspase 8 molecule” is defined as an inducible Caspase 1, 3, or 8, respectively. The term iCaspase 1, iCaspase 3, or iCaspase 8, embraces iCaspase 1, 3, or 8 nucleic acids, iCaspase 1, 3, or 8 polypeptides and/or iCaspase 1, 3, or 8 expression vectors, respectively. The term also encompasses either the natural Caspase iCaspase-1, -3, or -8 nucleotide or amino acid sequence, respectively, or a truncated sequence that is lacking the CARD domain. By “wild type” Caspase-9 in the context of the experimental details provided herein, is meant the Caspase-9 molecule lacking the CARD domain.

Modified Caspase-9 polypeptides comprise at least one amino acid substitution that affects basal activity or IC₅₀, in a chimeric polypeptide comprising the modified Caspase-9 polypeptide. Methods for testing basal activity and IC₅₀ are discussed herein. Non-modified Caspase-9 polypeptides do not comprise this type of amino acid substitution. Both modified and non-modified Caspase-9 polypeptides may be truncated, for example, to remove the CARD domain.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

The amino acid residue numbers referred to herein reflect the amino acid position in the non-truncated and non-modified Caspase-9 polypeptide, for example, that of SEQ ID NO: 9. SEQ ID NO: 9 provides an amino acid sequence for the truncated Caspase-9 polypeptide, which does not include the CARD domain. Thus SEQ ID NO: 9 commences at amino acid residue number 135, and ends at amino acid residue number 416, with reference to the full length Caspase-9 amino acid sequence. Those of ordinary skill in the art may align the sequence with other sequences of Caspase-9 polypeptides to, if desired, correlate the amino acid residue number, for example, using the sequence alignment methods discussed herein.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

As used herein, the term “ex vivo” refers to “outside” the body. The terms “ex vivo” and “in vitro” can be used interchangeably herein.

As used herein, the term “functionally equivalent,” as it relates to Caspase-9, or truncated Caspase-9, for example, refers to a Caspase-9 nucleic acid fragment, variant, or analog, refers to a nucleic acid that codes for a Caspase-9 polypeptide, or a Caspase-9 polypeptide, that stimulates an apoptotic response. “Functionally equivalent” refers, for example, to a Caspase-9 polypeptide that is lacking the CARD domain, but is capable of inducing an apoptotic cell response. When the term “functionally equivalent” is applied to other nucleic acids or polypeptides, such as, for example, CD19, the 5′LTR, the multimeric ligand binding region, or CD3, it refers to fragments, variants, and the like that have the same or similar activity as the reference polypeptides of the methods herein.

As used herein, the term “gene” is defined as a functional protein, polypeptide, or peptide-encoding unit. As will be understood, this functional term includes genomic sequences, cDNA sequences, and smaller engineered gene segments that express, or are adapted to express, proteins, polypeptides, domains, peptides, fusion proteins, and mutants.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

The term “immunogenic composition” or “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunocompromised” as used herein is defined as a subject that has reduced or weakened immune system. The immunocompromised condition may be due to a defect or dysfunction of the immune system or to other factors that heighten susceptibility to infection and/or disease. Although such a categorization allows a conceptual basis for evaluation, immunocompromised individuals often do not fit completely into one group or the other. More than one defect in the body's defense mechanisms may be affected. For example, individuals with a specific T-lymphocyte defect caused by HIV may also have neutropenia caused by drugs used for antiviral therapy or be immunocompromised because of a breach of the integrity of the skin and mucous membranes. An immunocompromised state can result from indwelling central lines or other types of impairment due to intravenous drug abuse; or be caused by secondary malignancy, malnutrition, or having been infected with other infectious agents such as tuberculosis or sexually transmitted diseases, e.g., syphilis or hepatitis.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PORT″, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

The term “transfection” and “transduction” are interchangeable and refer to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like.

As used herein, the term “syngeneic” refers to cells, tissues or animals that have genotypes that are identical or closely related enough to allow tissue transplant, or are immunologically compatible. For example, identical twins or animals of the same inbred strain. Syngeneic and isogeneic can be used interchangeably.

The terms “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

By “T cell activation molecule” is meant a polypeptide that, when incorporated into a T cell expressing a chimeric antigen receptor, enhances activation of the T cell. Examples include, but are not limited to, ITAM-containing, Signal 1 conferring molecules such as, for example, CD3ζ polypeptide, and Fc receptor gamma, such as, for example, Fc epsilon receptor gamma (FcεR1γ) subunit (Haynes, N. M., et al. J. Immunol. 166:182-7 (2001)) J. Immunology).

As used herein, the term “under transcriptional control” or “operatively linked” is defined as the promoter is in the correct location and orientation in relation to the nucleic acid to control RNA polymerase initiation and expression of the gene.

As used herein, the terms “treatment”, “treat”, “treated”, or “treating” refer to prophylaxis and/or therapy.

As used herein, the term “vaccine” refers to a formulation that contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

In some embodiments, the nucleic acid is contained within a viral vector. In certain embodiments, the viral vector is a retroviral vector. In certain embodiments, the viral vector is an adenoviral vector or a lentiviral vector. It is understood that in some embodiments, the antigen-presenting cell is contacted with the viral vector ex vivo, and in some embodiments, the antigen-presenting cell is contacted with the viral vector in vivo.

### Hematopoietic Stem Cells and Cell Therapy

Hematopoietic stem cells include hematopoietic progenitor cells, immature, multipotent cells that can differentiate into mature blood cell types. These stem cells and progenitor cells may be isolated from bone marrow and umbilical cord blood, and, in some cases, from peripheral blood. Other stem and progenitor cells include, for example, mesenchymal stromal cells, embryonic stem cells, and inducible pluripotent stem cells.

Bone marrow derived mesenchymal stromal cells (MSCs) have been defined as a fraction of mononuclear bone marrow cells that adhere to plastic culture dishes in standard culture conditions, are negative for hematopoietic lineage markers and positive for CD73, CD90 and CD105, and able to differentiate in vitro into adipocytes, osteoblasts, and chondroblasts. While one physiologic role is presumed to be the support of hematopoiesis, several reports have also established that MSCs are able to incorporate and possibly proliferate in areas of active growth, such as cicatricial and neoplastic tissues, and to home to their native microenvironment and replace the function of diseased cells. Their differentiation potential and homing ability make MSCs attractive vehicles for cellular therapy, either in their native form for regenerative applications, or through their genetic modification for delivery of active biological agents to specific microenvironments such as diseased bone marrow or metastatic deposits. In addition, MSCs possess potent intrinsic immunosuppressive activity, and to date have found their most frequent application in the experimental treatment of graft-versus-host disease and autoimmune disorders (Pittenger, M. F., et al. (1999). Science 284: 143-147; Dominici, M., et al. (2006). Cytotherapy 8: 315-317; Prockop, D. J. (1997). Science 276: 71-74; Lee, R. H., et al. (2006). Proc Natl Acad Sci USA 103: 17438-17443; Studeny, M., et al., (2002). Cancer Res 62: 3603-3608; Studeny, M., et al. (2004). J Natl Cancer Inst 96: 1593-1603; Horwitz, E. M., et al. (1999). Nat Med 5: 309-313; Chamberlain, G., et al., (2007). Stem Cells 25: 2739-2749; Phinney, D. G., and Prockop, D. J. (2007). Stem Cells 25: 2896-2902; Horwitz, E. M., et al. (2002). Proc Natl Acad Sci USA 99: 8932-8937; Hall, B., et al., (2007). Int J Hematol 86: 8-16; Nauta, A. J., and Fibbe, W. E. (2007). Blood 110: 3499-3506; Le Blanc, K., et al. (2008). Lancet 371: 1579-1586; Tyndall, A., and Uccelli, A. (2009). Bone Marrow Transplant).

MSCs have been infused in hundreds of patients with minimal reported side effects. However, follow-up is limited, long term side effects are unknown, and little is known of the consequences that will be associated with future efforts to induce their in vivo differentiation, for example to cartilage or bone, or to genetically modify them to enhance their functionality. Several animal models have raised safety concerns. For instance, spontaneous osteosarcoma formation in culture has been observed in murine derived MSCs. Furthermore, ectopic ossification and calcification foci have been discussed in mouse and rat models of myocardial infarction after local injection of MSC, and their proarrhythmic potential has also been apparent in co-culture experiments with neonatal rat ventricular myocytes. Moreover, bilateral diffuse pulmonary ossification has been observed after bone marrow transplant in a dog, presumably due to the transplanted stromal components (Horwitz, E. M., et al., (2007). Biol Blood Marrow Transplant 13: 53-57; Tolar, J., et al. (2007). Stem Cells 25: 371-379; Yoon, Y.-S., et al., (2004). Circulation 109: 3154-3157; Breitbach, M., et al. (2007). Blood 110: 1362-1369; Chang, M. G., et al. (2006). Circulation 113: 1832-1841; Sale, G. E., and Storb, R. (1983). Exp Hematol 11: 961-966).

In another example of cell therapy, T cells transduced with a nucleic acid encoding a chimeric antigen receptor have been administered to patients to treat cancer (Zhong, X.-S., (2010) Molecular Therapy 18:413-420). Chimeric antigen receptors (CARs) are artificial receptors designed to convey antigen specificity to T cells without the requirement for MHC antigen presentation. They include an antigen-specific component, a transmembrane component, and an intracellular component selected to activate the T cell and provide specific immunity. Chimeric antigen receptor-expressing T cells may be used in various therapies, including cancer therapies. Costimulating polypeptides may be used to enhance the activation of CAR-expressing T cells against target antigens, and therefore increase the potency of adoptive immunotherapy.

For example, T cells expressing a chimeric antigen receptor based on the humanized monoclonal antibody Trastuzumab (Herceptin) has been used to treat cancer patients. Adverse events are possible, however, and in at least one reported case, the therapy had fatal consequences to the patient (Morgan, R. A., et al., (2010) Molecular Therapy 18:843-851). Transducing the cells with a chimeric Caspase-9-based safety switch as presented herein, would provide a safety switch that could stop the adverse event from progressing. Therefore, in some embodiments are provided nucleic acids, cells, and methods wherein the modified T cell also expresses an inducible Caspase-9 polypeptide. If there is a need, for example, to reduce the number of chimeric antigen receptor modified T cells, an inducible ligand may be administered to the patient, thereby inducing apoptosis of the modified T cells.

The antitumor efficacy from immunotherapy with T cells engineered to express chimeric antigen receptors (CARs) has steadily improved as CAR molecules have incorporated additional signaling domains to increase their potency. T cells transduced with first generation CARs, containing only the CD3 intracellular signaling molecule, have demonstrated poor persistence and expansion in vivo following adoptive transfer (Till B G, Jensen M C, Wang J, et al: CD20-specific adoptive immunotherapy for lymphoma using a chimeric antigen receptor with both CD28 and 4-1BB domains: pilot clinical trial results. Blood 119:3940-50, 2012; Pule M A, Savoldo B, Myers G D, et al: Virus-specific T cells engineered to coexpress tumor-specific receptors: persistence and antitumor activity in individuals with neuroblastoma. Nat Med 14:1264-70, 2008; Kershaw M H, Westwood J A, Parker L L, et al: A phase 1 study on adoptive immunotherapy using gene-modified T cells for ovarian cancer. Clin Cancer Res 12:6106-15, 2006), as tumor cells often lack the requisite costimulating molecules necessary for complete T cell activation. Second generation CAR T cells were designed to improve proliferation and survival of the cells. Second generation CAR T cells that incorporate the intracellular costimulating domains from either CD28 or 4-1BB (Carpenito C, Milone M C, Hassan R, et al: Control of large, established tumor xenografts with genetically retargeted human T cells containing CD28 and CD137 domains. Proc Natl Acad Sci USA 106:3360-5, 2009; Song D G, Ye Q, Poussin M, et al: CD27 costimulation augments the survival and antitumor activity of redirected human T cells in vivo. Blood 119:696-706, 2012), show improved survival and in vivo expansion following adoptive transfer, and more recent clinical trials using anti-CD19 CAR-modified T cells containing these costimulating molecules have shown remarkable efficacy for the treatment of CD19⁺ leukemia. (Kalos M, Levine B L, Porter D L, et al: T cells with chimeric antigen receptors have potent antitumor effects and can establish memory in patients with advanced leukemia. Sci Transl Med 3:95ra73, 2011; Porter D L, Levine B L, Kalos M, et al: Chimeric antigen receptor-modified T cells in chronic lymphoid leukemia. N Engl J Med 365:725-33, 2011; Brentjens R J, Davila M L, Riviere I, et al: CD19-targeted T cells rapidly induce molecular remissions in adults with chemotherapy-refractory acute lymphoblastic leukemia. Sci Transl Med 5:177ra38, 2013).

While others have explored additional signaling molecules from tumor necrosis factor (TNF)-family proteins, such as OX40 and 4-1BB, called “third generation” CART cells, (Finney H M, Akbar A N, Lawson A D: Activation of resting human primary T cells with chimeric receptors: costimulation from CD28, inducible costimulator, CD134, and CD137 in series with signals from the TCR zeta chain. J Immunol 172:104-13, 2004; Guedan S, Chen X, Madar A, et al: ICOS-based chimeric antigen receptors program bipolar TH17/TH1 cells. Blood, 2014), other molecules which induce T cell signaling distinct from the CD3ζ nuclear factor of activated T cells (NFAT) pathway may provide necessary costimulation for T cell survival and proliferation, and possibly endow CAR T cells with additional, valuable functions, not supplied by more conventional costimulating molecules. Some second and third-generation CAR T cells have been implicated in patient deaths, due to cytokine storm and tumor lysis syndrome caused by highly activated T cells.

By “chimeric antigen receptor” or “CAR” is meant, for example, a chimeric polypeptide which comprises a polypeptide sequence that recognizes a target antigen (an antigen-recognition domain) linked to a transmembrane polypeptide and intracellular domain polypeptide selected to activate the T cell and provide specific immunity. The antigen-recognition domain may be a single-chain variable fragment (scFv), or may, for example, be derived from other molecules such as, for example, a T cell receptor or Pattern Recognition Receptor. The intracellular domain comprises at least one polypeptide which causes activation of the T cell, such as, for example, but not limited to, CD3 zeta, and, for example, co-stimulatory molecules, for example, but not limited to, CD28, OX40 and 4-1BB. The term “chimeric antigen receptor” may also refer to chimeric receptors that are not derived from antibodies, but are chimeric T cell receptors. These chimeric T cell receptors may comprise a polypeptide sequence that recognizes a target antigen, where the recognition sequence may be, for example, but not limited to, the recognition sequence derived from a T cell receptor or an scFv. The intracellular domain polypeptides are those that act to activate the T cell. Chimeric T cell receptors are discussed in, for example, Gross, G., and Eshar, Z., FASEB Journal 6:3370-3378 (1992), and Zhang, Y., et al., PLOS Pathogens 6:1-13 (2010).

In one type of chimeric antigen receptor (CAR), the variable heavy (VH) and light (VL) chains for a tumor-specific monoclonal antibody are fused in-frame with the CD3 zeta chain (ζ) from the T cell receptor complex. The VH and VL are generally connected together using a flexible glycine-serine linker, and then attached to the transmembrane domain by a spacer (CH2CH3) to extend the scFv away from the cell surface so that it can interact with tumor antigens. Following transduction, T cells now express the CAR on their surface, and upon contact and ligation with a tumor antigen, signal through the CD3 zeta chain inducing cytotoxicity and cellular activation.

Investigators have noted that activation of T cells through CD3 zeta is sufficient to induce a tumor-specific killing, but is insufficient to induce T cell proliferation and survival. Early clinical trials using T cells modified with first generation CARs expressing only the zeta chain showed that gene-modified T cells exhibited poor survival and proliferation in vivo.

As co-stimulation through the B7 axis is necessary for complete T cell activation, investigators added the co-stimulating polypeptide CD28 signaling domain to the CAR construct. This region generally contains the transmembrane region (in place of the CD3 zeta version) and the YMNM motif for binding PI3K and Lck. In vivo comparisons between T cells expressing CARs with only zeta or CARs with both zeta and CD28 demonstrated that CD28 enhanced expansion in vivo, in part due to increased IL-2 production following activation. The inclusion of CD28 is called a 2nd generation CAR. The most commonly used costimulating molecules include CD28 and 4-1BB, which, following tumor recognition, can initiate a signaling cascade resulting in NF-κB activation, which promotes both T cell proliferation and cell survival.

The use of co-stimulating polypeptides 4-1BB or OX40 in CAR design has further improved T cell survival and efficacy. 4-1BB in particular appears to greatly enhance T cell proliferation and survival. This 3rd generation design (with 3 signaling domains) has been used in PSMA CARs (Zhong X S, et al., Mol Ther. 2010 February; 18(2):413-20) and in CD19 CARs, most notably for the treatment of CLL (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533). These cells showed impressive function in 3 patients, expanding more than a 1000-fold in vivo, and resulted in sustained remission in all three patients.

It is understood that by “derived” is meant that the nucleotide sequence or amino acid sequence may be derived from the sequence of the molecule. The intracellular domain comprises at least one polypeptide which causes activation of the T cell, such as, for example, but not limited to, CD3 zeta, and, for example, co-stimulatory molecules, for example, but not limited to, CD28, OX40 and 4-1BB.

T cell receptors are molecules composed of two different polypeptides that are on the surface of T cells. They recognize antigens bound to major histocompatibility complex molecules; upon recognition with the antigen, the T cell is activated. By “recognize” is meant, for example, that the T cell receptor, or fragment or fragments thereof, such as TCRα polypeptide and TCRβ together, is capable of contacting the antigen and identifying it as a target. TCRs may comprise α and β polypeptides, or chains. The α and β polypeptides include two extracellular domains, the variable and the constant domains. The variable domain of the α and β polypeptides has three complementarity determining regions (CDRs); CDR3 is considered to be the main CDR responsible for recognizing the epitope. The a polypeptide includes the V and J regions, generated by VJ recombination, and the β polypeptide includes the V, D, and J regions, generated by VDJ recombination. The intersection of the VJ regions and VDJ regions corresponds to the CDR3 region. TCRs are often named using the International Immunogenetics (IMGT) TCR nomenclature (IMGT Database, www.IMGT.org; Giudicelli, V., et al., IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

Chimeric T cell receptors may bind to, for example, antigenic polypeptides such as Bob-1, PRAME, and NY-ESO-1. (U.S. patent application Ser. No. 14/930,572, filed Nov. 2, 2015, titled “T Cell Receptors Directed Against Bob1 and Uses Thereof,” and U.S. Provisional Patent Application No. 62/130,884, filed Mar. 10, 2015, titled “T Cell Receptors Directed Against the Preferentially-Expressed Antigen of Melanoma and Uses Thereof, each of which incorporated by reference in its entirety herein).

In another example of cell therapy, T cells are modified so that they express a non-functional TGF-beta receptor, rendering them resistant to TGF-beta. This allows the modified T cells to avoid the cytotoxicity caused by TGF-beta, and allows the cells to be used in cellular therapy (Bollard, C. J., et al., (2002) Blood 99:3179-3187; Bollard, C. M., et al., (2004) J. Exptl. Med. 200:1623-1633). However, it also could result in a T cell lymphoma, or other adverse effect, as the modified T cells now lack part of the normal cellular control; these therapeutic T cells could themselves become malignant. Transducing these modified T cells with a chimeric Caspase-9-based safety switch as presented herein, would provide a safety switch that could avoid this result.

In other examples, Natural Killer cells are modified to express the membrane-targeting polypeptide. Instead of a chimeric antigen receptor, in certain embodiments, the heterologous membrane bound polypeptide is a NKG2D receptor. NKG2D receptors can bind to stress proteins (e.g. MICA/B) on tumor cells and can thereby activate NK cells. The extracellular binding domain can also be fused to signaling domains (Barber, A., et al., Cancer Res 2007; 67: 5003-8; Barber A, et al., Exp Hematol. 2008; 36:1318-28; Zhang T., et al., Cancer Res. 2007; 67:11029-36., and this could, in turn, be linked to FRB domains, analogous to FRB-linkered CARs. Moreover, other cell surface receptors, such as VEGF-R could be used as a docking site for FRB domains to enhance tumor-dependent clustering in the presence of hypoxia-triggered VEGF, found at high levels within many tumors.

Cells used in cellular therapy, that express a heterologous gene, such as a modified receptor, or a chimeric receptor, may be transduced with nucleic acid that encodes a chimeric Caspase-9-based safety switch before, after, or at the same time, as the cells are transduced with the heterologous gene.

### Haploidentical Stem Cell Transplantation

While stem cell transplantation has proven an effective means of treating a wide variety of diseases involving hematopoietic stem cells and their progeny, a shortage of histocompatible donors has proved a major impediment to the widest application of the approach. The introduction of large panels of unrelated stem cell donors and or cord blood banks has helped to alleviate the problem, but many patients remain unsuited to either source. Even when a matched donor can be found, the elapsed time between commencing the search and collecting the stem cells usually exceeds three months, a delay that may doom many of the neediest patients. Hence there has been considerable interest in making use of HLA haploidentical family donors. Such donors may be parents, siblings or second-degree relatives. The problem of graft rejection may be overcome by a combination of appropriate conditioning and large doses of stem cells, while graft versus host disease (GvHD) may be prevented by extensive T cell-depletion of the donor graft. The immediate outcomes of such procedures have been gratifying, with engraftment rate >90% and a severe GvHD rate of <10% for both adults and children even in the absence of post transplant immunosuppression. Unfortunately, the profound immunosuppression of the grafting procedure, coupled with the extensive T cell-depletion and HLA mismatching between donor and recipient lead to an extremely high rate of post-transplant infectious complications, and contributed to high incidence of disease relapse.

Donor T cell infusion is an effective strategy for conferring anti-viral and anti-tumor immunity following allogeneic stem cell transplantation. Simple addback of T cells to the patients after haploidentical transplantation, however, cannot work; the frequency of alloreactive T cells is several orders of magnitude higher than the frequency of, for example, virus specific T lymphocytes. Methods are being developed to accelerate immune reconstitution by administrating donor T cells that have first been depleted of alloreactive ceils. One method of achieving this is stimulating donor T cells with recipient EBV-transformed B lymphoblastoid cell lines (LCLs). Alloreactive T cells upregulate CD25 expression, and are eliminated by a CD25 Mab immunotoxin conjugate, RFT5-SMPT-dgA. This compound consists of a murine IgG1 anti-CD25 (IL-2 receptor alpha chain) conjugated via a hetero-bifunctional crosslinker [N-succinimidyloxycarbonyl-alpha-methyl-d-(2-pyridylthio) toluene] to chemically deglycosylated ricin A chain (dgA).

Treatment with CD25 immunotoxin after LCL stimulation depletes >90% of alloreactive cells. In a phase 1 clinical study, using CD25 immunotoxin to deplete alloreactive lymphocytes immune reconstitution after allodepleted donor T cells were infused at 2 dose levels into recipients of T-cell-depleted haploidentical SCT. Eight patients were treated at 10⁴ cells/kg/dose, and 8 patients received 10⁵ cells/kg/dose. Patients receiving 10⁵ cells/kg/dose showed significantly improved T-cell recovery at 3, 4, and 5 months after SCT compared with those receiving 10⁴ cells/kg/dose (P<0.05). Accelerated T-cell recovery occurred as a result of expansion of the effector memory (CD45RA(−)CCR-7(−)) population (P<0.05), suggesting that protective T-cell responses are likely to be long lived. T-cell-receptor signal joint excision circles (TRECs) were not detected in reconstituting T cells in dose-level 2 patients, indicating they are likely to be derived from the infused allodepleted cells. Spectratyping of the T cells at 4 months demonstrated a polyclonal Vbeta repertoire. Using tetramer and enzyme-linked immunospot (ELISpot) assays, cytomegalovirus (CMV)- and Epstein-Barr virus (EBV)-specific responses in 4 of 6 evaluable patients at dose level 2 as early as 2 to 4 months after transplantation, whereas such responses were not observed until 6 to 12 months in dose-level 1 patients. The incidence of significant acute (2 of 16) and chronic graft-versus-host disease (GvHD; 2 of 15) was low. These data demonstrate that allodepleted donor T cells can be safely used to improve T-cell recovery after haploidentical SCT. The amount of cells infused was subsequently escalated to 10⁶ cells/kg without evidence of GvHD.

Although this approach reconstituted antiviral immunity, relapse remained a major problem and 6 patients transplanted for high risk leukemia relapsed and died of disease. Higher T cell doses are therefore useful to reconstitute anti-tumor immunity and to provide the hoped-for anti-tumor effect, since the estimated frequency of tumor-reactive precursors is 1 to 2 logs less than frequency of viral-reactive precursors. However, in some patients, these doses of cells will be sufficient to trigger GvHD even after allodepletion (Hurley C K, et al., Biol Blood Marrow Transplant 2003; 9:610-615; Dey B R, et al., Br. J Haematol. 2006; 135:423-437; Aversa F, et al., N Engl J Med 1998; 339:1186-1193; Aversa F, et al., J Clin. Oncol. 2005; 23:3447-3454; Lang P, Mol. Dis. 2004; 33:281-287; Kolb H J, et al., Blood 2004; 103:767-776; Gottschalk S, et al., Annu. Rev. Med 2005; 56:29-44; Bleakley M, et al., Nat. Rev. Cancer 2004; 4:371-380; Andre-Schmutz I, et al., Lancet 2002; 360:130-137; Solomon S R, et al., Blood 2005; 106:1123-1129; Amrolia P J, et al., Blood 2006; 108:1797-1808; Amrolia P J, et al., Blood 2003; Ghetie V, et al., J Immunol Methods 1991; 142:223-230; Molldrem J J, et al., Cancer Res 1999; 59:2675-2681; Rezvani K, et al., Clin. Cancer Res. 2005; 1 1:8799-8807; Rezvani K, et al., Blood 2003; 102:2892-2900).

### Graft Versus Host Disease (GvHD)

Graft versus Host Disease is a condition that sometimes occurs after the transplantation of donor immunocompetent cells, for example, T cells, into a recipient. The transplanted cells recognize the recipient's cells as foreign, and attack and destroy them. This condition can be a dangerous effect of T cell transplantation, especially when associated with haploidentical stem cell transplantation. Sufficient T cells should be infused to provide the beneficial effects, such as, for example, the reconstitution of an immune system and the graft anti-tumor effect. But, the number of T cells that can be transplanted can be limited by the concern that the transplant will result in severe graft versus host disease.

Graft versus Host Disease may be staged as indicated in the following tables:

### Staging

| | Stage 0 | Stage 1 | Stage 2 | Stage 3 | Stage 4 |
|---|---|---|---|---|---|
| Skin | No rash | Rash <25% | 25-50% | >50% | Plus bullae and |
| | | BSA | | Generalized | desquamation |
| | | | | erythroderma | |
| Gut | <500 mL | 501-1000 | 1001-1500 | >1500 | Severe |
| (for | diarrhea/day | mL/day | mL/day | mL/day >15 | abdominal pain |
| pediatric | | 5 cc/kg-10 | 10 cc/kg-15 | cc/kg/day | and ileus |
| patients) | | cc/kg/day | cc/kg/day | | |
| UGI | | Severe | | | |
| | | nausea/vomiting | | | |
| Liver | Bilirubins | 2.1-3 mg/di | 3.1-6 mg/di | 6.1-15 mg/di | >15 mg/di |
| | 2 mg/di | | | | |

Acute GvHD grading may be performed by the consensus conference criteria (Przepiorka D et al., 1994 Consensus Conference on Acute GVHD Grading. Bone Marrow Transplant 1995; 15:825-828).

### Grading Index of Acute GvHD

| | Skin | Liver | Gut | Upper GI |
|---|---|---|---|---|
| 0 | None and | None and | None and | None |
| I | Stage 1-2 and | None and | None | None |
| II | Stage 3 and/or | Stage 1 and/or | Stage 1 and/or | Stage 1 |
| III | None-Stage 3 | Stage 2-3 or | Stage 2-4 | N/A |
| | with | | | |
| IV | Stage 4 or | Stage 4 | N/A | N/A |

### Inducible Caspase-9 as a “Safety Switch” for Cell Therapy and for Genetically Engineered Cell Transplantation

By reducing the effect of graft versus host disease is meant, for example, a decrease in the GvHD symptoms so that the patient may be assigned a lower level stage, or, for example, a reduction of a symptom of graft versus host disease by at least 20%, 30%, 40%, 50%, 60%, 70%, 80%, 90%, 95%, or 99%. A reduction in the effect of graft versus host disease may also be measured by detection of a reduction in activated T cells involved in the GvHD reaction, such as, for example, a reduction of cells that express the marker protein, for example CD19, and express CD3 (CD3⁺CD19⁺ cells, for example) by at least 30%, 40%, 50%, 60%, 70%, 75%, 80%, 85%, 90%, 95%, or 99%.

Provided herein is an alternative suicide gene strategy that is based on human proapoptotic molecules fused with an FKBP variant that is optimized to bind a chemical inducer of dimerization (CID). Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). AP1903 is a synthetic molecule that has proven safe in healthy volunteers (Iuliucci J D, et al., J Clin Pharmacol. 2001, 41:870-879). Administration of this small molecule results in cross-linking and activation of the proapoptotic target molecules. The application of this inducible system in human T lymphocytes has been explored using Fas or the death effector domain (DED) of the Fas-associated death domain-containing protein (FADD) as proapoptotic molecules. Up to 90% of T cells transduced with these inducible death molecules underwent apoptosis after administration of CID (Thomis D C, et al., Blood. 2001, 97:1249-1257; Spencer D M, et al., Curr Biol. 1996, 6: 839-847; Fan L, et al., Hum Gene Ther. 1999, 10: 2273-2285; Berger C, et al., Blood. 2004, 103:1261-1269; Junker K, et al., Gene Ther. 2003, 10:1189-197). This suicide gene strategy may be used in any appropriate cell used for cell therapy including, for example, hematopoietic stem cells, and other progenitor cells, including, for example, mesenchymal stromal cells, embryonic stem cells, and inducible pluripotent stem cells. AP20187 and AP1950, a synthetic version of AP1903, may also be used as the ligand inducer. (Amara J F (97) PNAS 94:10618-23, Clontech Laboratories-Takara Bio).

Therefore, this safety switch, catalyzed by Caspase-9, may be used where there is a condition in the cell therapy patient that requires the removal of the transfected or transduced therapeutic cells. Conditions where the cells may need to be removed include, for example, GvHD, inappropriate differentiation of the cells into more mature cells of the wrong tissue or cell type, and other toxicities. To activate the Caspase-9 switch in the case of inappropriate differentiation, it is possible to use tissue specific promoters. For example, where a progenitor cell differentiates into bone and fat cells, and the fat cells are not desired, the vector used to transfect or transduce the progenitor cell may have a fat cell specific promoter that is operably linked to the Caspase-9 nucleotide sequence. In this way, should the cells differentiate into fat cells, upon administration of the multimer ligand, apoptosis of the inappropriately differentiated fat cells should result. The methods may be used, for example, for any disorder that can be alleviated by cell therapy, including cancer, cancer in the blood or bone marrow, other blood or bone marrow borne diseases such as sickle cell anemia and metachromic leukodystrophy, and any disorder that can be alleviated by a stem cell transplantation, for example blood or bone marrow disorders such as sickle cell anemia or metachromal leukodystrophy.

The efficacy of adoptive immunotherapy may be enhanced by rendering the therapeutic T cells resistant to immune evasion strategies employed by tumor cells. In vitro studies have shown that this can be achieved by transduction with a dominant-negative receptor or an immunomodulatory cytokine (Bollard C M, et al., Blood. 2002, 99:3179-3187: Wagner H J, et al., Cancer Gene Ther. 2004, 11:81-91). Moreover, transfer of antigen-specific T-cell receptors allows for the application of T-cell therapy to a broader range of tumors (Pule M, et al., Cytotherapy. 2003, 5:211-226; Schumacher T N, Nat Rev Immunol. 2002, 2:512-519). A suicide system for engineered human T cells was developed and tested to allow their subsequent use in clinical studies. Caspase-9 has been modified and shown to be stably expressed in human T lymphocytes without compromising their functional and phenotypic characteristics while demonstrating sensitivity to CID, even in T cells that have upregulated antiapoptotic molecules. (Straathof, K. C., et al., 2005, Blood 105:4248-54).

In genetically modified cells used for gene therapy, the gene may be a heterologous polynucleotide sequence derived from a source other than the cell that is used to express the gene. The gene is derived from a prokaryotic or eukaryotic source such as a bacterium, a virus, yeast, a parasite, a plant, or even an animal. The heterologous DNA also is derived from more than one source, i.e., a multigene construct or a fusion protein. The heterologous DNA also may include a regulatory sequence, which is derived from one source and the gene from a different source. Or, the heterologous DNA may include regulatory sequences that are used to change the normal expression of a cellular endogenous gene.

### Other Caspase Molecules

Caspase polypeptides other than Caspase-9 that may be encoded by the chimeric polypeptides of the current technology include, for example, Caspase-1, Caspase-3, and Caspase-8. Discussions of these Caspase polypeptides may be found in, for example, MacCorkle, R. A., et al., Proc. Natl. Acad. Sci. U.S.A. (1998) 95:3655-3660; and Fan, L., et al. (1999) Human Gene Therapy 10:2273-2285).

### Engineering Expression Constructs

Expression constructs encode a multimeric ligand binding region and a Caspase-9 polypeptide, or, in certain embodiments a multimeric ligand binding region and a Caspase-9 polypeptide linked to a marker polypeptide, all operatively linked. In general, the term “operably linked” is meant to indicate that the promoter sequence is functionally linked to a second sequence, wherein, for example, the promoter sequence initiates and mediates transcription of the DNA corresponding to the second sequence. The Caspase-9 polypeptide may be full length or truncated. In certain embodiments, the marker polypeptide is linked to the Caspase-9 polypeptide. For example, the marker polypeptide may be linked to the Caspase-9 polypeptide via a polypeptide sequence, such as, for example, a cleavable 2A-like sequence. The marker polypeptide may be, for example, CD19, or may be, for example, a heterologous protein, selected to not affect the activity of the chimeric caspase polypeptide.

In some embodiments, the polynucleotide may encode the Caspase-9 polypeptide and a heterologous protein, which may be, for example a marker polypeptide and may be, for example, a chimeric antigen receptor. The heterologous polypeptide, for example, the chimeric antigen receptor, may be linked to the Caspase-9 polypeptide via a polypeptide sequence, such as, for example, a cleavable 2A-like sequence.

In certain examples, a nucleic acid comprising a polynucleotide coding for a chimeric antigen receptor is included in the same vector, such as, for example, a viral or plasmid vector, as a polynucleotide coding for a second polypeptide. This second polypeptide may be, for example, a caspase polypeptide, as discussed herein, or a marker polypeptide. In these examples, the construct may be designed with one promoter operably linked to a nucleic acid comprising a polynucleotide coding for the two polypeptides, linked by a cleavable 2A polypeptide. In this example, the first and second polypeptides are separated during translation, resulting in a chimeric antigen receptor polypeptide, and the second polypeptide. In other examples, the two polypeptides may be expressed separately from the same vector, where each nucleic acid comprising a polynucleotide coding for one of the polypeptides is operably linked to a separate promoter. In yet other examples, one promoter may be operably linked to the two nucleic acids, directing the production of two separate RNA transcripts, and thus two polypeptides. Therefore, the expression constructs discussed herein may comprise at least one, or at least two promoters. 2A-like sequences, or “cleavable” 2A sequences, are derived from, for example, many different viruses, including, for example, from Thosea asigna. These sequences are sometimes also known as “peptide skipping sequences.” When this type of sequence is placed within a cistron, between two peptides that are intended to be separated, the ribosome appears to skip a peptide bond, in the case of Thosea asigna sequence, the bond between the Gly and Pro amino acids is omitted. This leaves two polypeptides, in this case the Caspase-9 polypeptide and the marker polypeptide. When this sequence is used, the peptide that is encoded 5′ of the 2A sequence may end up with additional amino acids at the carboxy terminus, including the Gly residue and any upstream in the 2A sequence. The peptide that is encoded 3′ of the 2A sequence may end up with additional amino acids at the amino terminus, including the Pro residue and any downstream in the 2A sequence. “2A” or “2A-like” sequences are part of a large family of peptides that can cause peptide bond-skipping. Various 2A sequences have been characterized (e.g., F2A, P2A, T2A), and are examples of 2A-like sequences that may be used in the polypeptides of the present application. In certain embodiments, the 2A linker comprises the amino acid sequence of SEQ ID NO: 614; in certain embodiments the 2A linker consists of the amino acid sequence of SEQ ID NO: 614. In some embodiments, the 2A linker comprises the amino acid sequence of SEQ ID NO: 998; in some embodiments the 2A linker consists of the amino acid sequence of SEQ ID NO: 998. In certain embodiments, the 2A linker further comprises a GSG amino acid sequence (SEQ ID NO: 151) at the amino terminus of the polypeptide, in other embodiments, the 2A linker comprises a GSGPR amino acid sequence (SEQ ID NO: 925) at the amino terminus of the polypeptide. Thus, by a “2A” sequence, the term may refer to the 2A sequence as listed herein, or may also refer to a 2A sequence as listed herein further comprising a GSG (SEQ ID NO: 151) or GSGPR sequence (SEQ ID NO: 925) at the amino terminus of the linker.

The expression construct may be inserted into a vector, for example a viral vector or plasmid. The steps of the methods provided may be performed using any suitable method; these methods include, without limitation, methods of transducing, transforming, or otherwise providing nucleic acid to the antigen-presenting cell, presented herein. In some embodiments, the truncated Caspase-9 polypeptide is encoded by the nucleotide sequence of SEQ ID NO 8, SEQ ID NO: 23, SEQ ID NO: 25, SEQ ID NO: 27, or a functionally equivalent fragment thereof, with or without DNA linkers, or has the amino acid sequence of SEQ ID NO: 9, SEQ ID NO: 24, SEQ ID NO: 26, or SEQ ID NO: 28 or a functionally equivalent fragment thereof. In some embodiments, the CD19 polypeptide is encoded by the nucleotide sequence of SEQ ID NO 14, or a functionally equivalent fragment thereof, with or without DNA linkers, or has the amino acid sequence of SEQ ID NO: 15, or a functionally equivalent fragment thereof. A functionally equivalent fragment of the Caspase-9 polypeptide has substantially the same ability to induce apoptosis as the polypeptide of SEQ ID NO: 9, with at least 50%, 60%, 70%, 80%, 90%, or 95% of the activity of the polypeptide of SEQ ID NO: 9. A functionally equivalent fragment of the CD19 polypeptide has substantially the same ability as the polypeptide of SEQ ID No: 15, to act as a marker to be used to identify and select transduced or transfected cells, with at least 50%, 60%, 70%, 80%, 90%, or 95% of the marker polypeptide being detected when compared to the polypeptide of SEQ ID NO: 15, using standard detection techniques.

More particularly, more than one ligand binding domain or multimerizing region may be used in the expression construct. Yet further, the expression construct contains a membrane-targeting sequence. Appropriate expression constructs may include a co-stimulatory polypeptide element on either side of the above FKBP ligand binding elements.

In certain examples, the polynucleotide coding for the inducible caspase polypeptide is included in the same vector, such as, for example, a viral or plasmid vector, as a polynucleotide coding for a chimeric antigen receptor. In these examples, the construct may be designed with one promoter operably linked to a nucleic acid comprising a nucleotide sequence coding for the two polypeptides, linked by a cleavable 2A polypeptide. In this example, the first and second polypeptides are cleaved after expression, resulting in a chimeric antigen receptor polypeptide and an inducible Caspase-9 polypeptide. In other examples, the two polypeptides may be expressed separately from the same vector, where each nucleic acid comprising a nucleotide sequence coding for one of the polypeptides is operably linked to a separate promoter. In yet other examples, one promoter may be operably linked to the two nucleic acids, directing the production of two separate RNA transcripts, and thus two polypeptides. Therefore, the expression constructs discussed herein may comprise at least one, or at least two promoters.

In yet other examples, two polypeptides may be expressed in a cell using two separate vectors. The cells may be co-transfected or co-transformed with the vectors, or the vectors may be introduced to the cells at different times.

### Ligand Binding Regions

The ligand binding (“dimerization”) domain, or multimerizing region, of the expression construct can be any convenient domain that will allow for induction using a natural or unnatural ligand, for example, an unnatural synthetic ligand. The multimerizing region can be internal or external to the cellular membrane, depending upon the nature of the construct and the choice of ligand. A wide variety of ligand binding proteins, including receptors, are known, including ligand binding proteins associated with the cytoplasmic regions indicated above. As used herein the term “ligand binding domain” can be interchangeable with the term “receptor”. Of particular interest are ligand binding proteins for which ligands (for example, small organic ligands) are known or may be readily produced. These ligand binding domains or receptors include the FKBPs and cyclophilin receptors, the steroid receptors, the tetracycline receptor, the other receptors indicated above, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In certain embodiments, the ligand binding region is selected from the group consisting of FKBP ligand binding region, cyclophilin receptor ligand binding region, steroid receptor ligand binding region, cyclophilin receptors ligand binding region, and tetracycline receptor ligand binding region. Often, the ligand binding region comprises a FᵥFᵥₗₛ sequence. Sometimes, the FᵥFᵥₗₛ sequence further comprises an additional F_{v′} sequence. Examples include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)).

For the most part, the ligand binding domains or receptor domains will be at least about 50 amino acids, and fewer than about 350 amino acids, usually fewer than 200 amino acids, either as the natural domain or truncated active portion thereof. The binding domain may, for example, be small (<25 kDa, to allow efficient transfection in viral vectors), monomeric, nonimmunogenic, have synthetically accessible, cell permeable, nontoxic ligands that can be configured for dimerization.

The receptor domain can be intracellular or extracellular depending upon the design of the expression construct and the availability of an appropriate ligand. For hydrophobic ligands, the binding domain can be on either side of the membrane, but for hydrophilic ligands, particularly protein ligands, the binding domain will usually be external to the cell membrane, unless there is a transport system for internalizing the ligand in a form in which it is available for binding. For an intracellular receptor, the construct can encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

The portion of the expression construct encoding the receptor can be subjected to mutagenesis for a variety of reasons. The mutagenized protein can provide for higher binding affinity, allow for discrimination by the ligand of the naturally occurring receptor and the mutagenized receptor, provide opportunities to design a receptor-ligand pair, or the like. The change in the receptor can involve changes in amino acids known to be at the binding site, random mutagenesis using combinatorial techniques, where the codons for the amino acids associated with the binding site or other amino acids associated with conformational changes can be subject to mutagenesis by changing the codon(s) for the particular amino acid, either with known changes or randomly, expressing the resulting proteins in an appropriate prokaryotic host and then screening the resulting proteins for binding.

Antibodies and antibody subunits, e.g., heavy or light chain, particularly fragments, more particularly all or part of the variable region, or fusions of heavy and light chain to create high-affinity binding, can be used as the binding domain. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen). Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding domain is known and there is a useful ligand for binding.

### Oligomerization

The transduced signal will normally result from ligand-mediated oligomerization of the chimeric protein molecules, i.e., as a result of oligomerization following ligand binding, although other binding events, for example allosteric activation, can be employed to initiate a signal. The construct of the chimeric protein will vary as to the order of the various domains and the number of repeats of an individual domain.

For multimerizing the receptor, the ligand for the ligand binding domains/receptor domains of the chimeric surface membrane proteins will usually be multimeric in the sense that it will have at least two binding sites, with each of the binding sites capable of binding to the ligand receptor domain. By “multimeric ligand binding region” is meant a ligand binding region that binds to a multimeric ligand. The term “multimeric ligands” include dimeric ligands. A dimeric ligand will have two binding sites capable of binding to the ligand receptor domain. Desirably, the subject ligands will be a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively, higher orders of the ligands, e.g., trimeric can be used. For embodiments involving unnatural receptors, e.g., antibody subunits, modified antibody subunits, single chain antibodies comprised of heavy and light chain variable regions in tandem, separated by a flexible linker domain, or modified receptors, and mutated sequences thereof, and the like, any of a large variety of compounds can be used. A significant characteristic of these ligand units is that each binding site is able to bind the receptor with high affinity and they are able to be dimerized chemically. Also, methods are available to balance the hydrophobicity/hydrophilicity of the ligands so that they are able to dissolve in serum at functional levels, yet diffuse across plasma membranes for most applications.

In certain embodiments, the present methods utilize the technique of chemically induced dimerization (CID) to produce a conditionally controlled protein or polypeptide. In addition to this technique being inducible, it also is reversible, due to the degradation of the labile dimerizing agent or administration of a monomeric competitive inhibitor. The CID system uses synthetic bivalent ligands to rapidly crosslink signaling molecules that are fused to ligand binding domains. This system has been used to trigger the oligomerization and activation of cell surface (Spencer, D. M., et al., Science, 1993. 262: p. 1019-1024; Spencer D. M. et al., Curr Biol 1996, 6:839-847; Blau, C. A. et al., Proc Natl Acad. Sci. USA 1997, 94:3076-3081), or cytosolic proteins (Luo, Z. et al., Nature 1996, 383:181-185; MacCorkle, R. A. et al., Proc Natl Acad Sci USA 1998, 95:3655-3660), the recruitment of transcription factors to DNA elements to modulate transcription (Ho, S. N. et al., Nature 1996, 382:822-826; Rivera, V. M. et al., Nat. Med. 1996, 2:1028-1032) or the recruitment of signaling molecules to the plasma membrane to stimulate signaling (Spencer D. M. et al., Proc. Natl. Acad. Sci. USA 1995, 92:9805-9809; Holsinger, L. J. et al., Proc. Natl. Acad. Sci. USA 1995, 95:9810-9814).

The CID system is based upon the notion that surface receptor aggregation effectively activates downstream signaling cascades. In the simplest embodiment, the CID system uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs to Caspase-9, one can stimulate Caspase-9 activity in a dimerizer drug-dependent, but ligand and ectodomain-independent manner. This provides the system with temporal control, reversibility using monomeric drug analogs, and enhanced specificity. The high affinity of third-generation AP20187/AP1903 CIDs for their binding domain, FKBP12, permits specific activation of the recombinant receptor in vivo without the induction of non-specific side effects through endogenous FKBP12. FKBP12 variants having amino acid substitutions and deletions, such as FKBP12v36, that bind to a dimerizer drug, may also be used. FKBP12 variants include, but are not limited to, those having amino acid substitutions at position 36, selected from the group consisting of valine, leucine, isoleuceine, and alanine. In addition, the synthetic ligands are resistant to protease degradation, making them more efficient at activating receptors in vivo than most delivered protein agents.

By FKBP12 is meant the wild type FKBP12 polypeptide, or analogs or derivatives thereof that may comprise amino acid substitutions, that maintains FKBP12 binding activity to rapamycin; FKBP12 polypeptides or polypeptide regions bind to rimiducid with at least 100 times less affinity than FKBP12v36 polypeptides. In some examples, the FKBP12 polypeptide binds to a ligand, such as rimiducid, with at least 100 times less affinity than an FKBP12 variant polypeptide consisting of the amino acid sequence of SEQ ID NO: 977.

By FKBP12 variant polypeptide if meant an FKBP12 polypeptide that binds to a ligand, such as rimiducid with at least 100 times more affinity than a wild type FKBP12 polypeptide, such as, for example, the wild type FKBP12 polypeptide consisting of the amino acid sequence of SEQ ID NO: 929.

The ligands used are capable of binding to two or more of the ligand binding domains. The chimeric proteins may be able to bind to more than one ligand when they contain more than one ligand binding domain. The ligand is typically a non-protein or a chemical. Exemplary ligands include, but are not limited to FK506 (e.g., FK1012).

Other ligand binding regions may be, for example, dimeric regions, or modified ligand binding regions with a wobble substitution, such as, for example, FKBP12(V36): The human 12 kDa FK506-binding protein with an F36 to V substitution, the complete mature coding sequence (amino acids 1-107), provides a binding site for synthetic dimerizer drug AP1903 (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein may also be used in the construct so that higher-order oligomers are induced upon cross-linking by AP1903.

FKBP12 variants may also be used in the FKBP12/FRB multimerizing regions. Variants used in these fusions, in some embodiments, will bind to rapamycin, or rapalogs, but will bind to less affinity to rimiducid than, for example, FKBP12v36. Examples of FKBP12 variants include those from many species, including, for example, yeast. In one embodiment, the FKBP12 variant is FKBP12.6 (calstablin).

Other heterodimers are contemplated in the present application. In one embodiment, a calcineurin-A polypeptide, or region may be used in place of the FRB multimerizing region. In some embodiments, the first unit of the first multimerizing region is a calcineurin-A polypeptide. In some embodiments, the first unit of the first multimerizing region is a calcineurin-A polypeptide region and the second unit of the first multimerizing region is a FKBP12 or FKBP12 variant multimerizing region. In some embodiments, the first unit of the first multimerizing region is a FKBP12 or FKBP12 variant multimerizing region and the second unit of the first multimerizing region is a calcineuring-A polypeptide region. In these embodiments, the first ligand comprises, for example, cyclosporine.

F36V′-FKBP: F36V′-FKBP is a codon-wobbled version of F36V-FKBP. It encodes the identical polypeptide sequence as F36V-FKPB but has only 62% homology at the nucleotide level. F36V′-FKBP was designed to reduce recombination in retroviral vectors (Schellhammer, P. F. et al., J. Urol. 157, 1731-5 (1997)). F36V′-FKBP was constructed by a PCR assembly procedure. The transgene contains one copy of F36V′-FKBP linked directly to one copy of F36V-FKBP.

In some embodiments, the ligand is a small molecule. The appropriate ligand for the selected ligand binding region may be selected. Often, the ligand is dimeric, sometimes, the ligand is a dimeric FK506 or a dimeric FK506-like analog. In certain embodiments, the ligand is AP1903 (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-di methoxyphenyl)propylidene]] ester, [2S-[1(R*),2R*[S*[S*[1(R*),2R*]]]]]-(9C1) CAS Registry Number: 195514-63-7; Molecular Formula: C78H98N4O20 Molecular Weight: 1411.65). In certain embodiments, the ligand is AP20187. In certain embodiments, the ligand is an AP20187 analog, such as, for example, AP1510. In some embodiments, certain analogs will be appropriate for the FKBP12, and certain analogs appropriate for the wobbled version of FKBP12. In certain embodiments, one ligand binding region is included in the chimeric protein. In other embodiments, two or more ligand binding regions are included. Where, for example, the ligand binding region is FKBP12, where two of these regions are included, one may, for example, be the wobbled version.

Other dimerization systems contemplated include the coumermycin/DNA gyrase B system. Coumermycin-induced dimerization activates a modified Raf protein and stimulating the MAP kinase cascade. See Farrar, M. A., et. Al., (1996) Nature 383, 178-181. In other embodiments, the abscisic acid (ABA) system developed by G R Crabtree and colleagues (Liang F S, et al., Sci Signal. 2011 Mar. 15; 4(164):rs2), may be used, but like DNA gyrase B, this relies on a foreign protein, which would be immunogenic.

### Membrane-Targeting

A membrane-targeting sequence or region provides for transport of the chimeric protein to the cell surface membrane, where the same or other sequences can encode binding of the chimeric protein to the cell surface membrane. Molecules in association with cell membranes contain certain regions that facilitate the membrane association, and such regions can be incorporated into a chimeric protein molecule to generate membrane-targeted molecules. For example, some proteins contain sequences at the N-terminus or C-terminus that are acylated, and these acyl moieties facilitate membrane association. Such sequences are recognized by acyltransferases and often conform to a particular sequence motif. Certain acylation motifs are capable of being modified with a single acyl moiety (often followed by several positively charged residues (e.g. human c-Src: M-G-S-N-K-S-K-P-K-D-A-S-Q-R-R-R (SEQ ID NO: 283)) to improve association with anionic lipid head groups) and others are capable of being modified with multiple acyl moieties. For example, the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys (SEQ ID NO: 284), where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can modified with C15 or C10 isoprenyl moieties, from the C-terminus of G-protein gamma subunits and other proteins (e.g., World Wide Web address ebi.ac.uk/interpro/DisplaylproEntry?ac=IPR001230) also can be utilized. These and other acylation motifs include, for example, those discussed in Gauthier-Campbell et al., Molecular Biology of the Cell 15: 2205-2217 (2004); Glabati et al., Biochem. J. 303: 697-700 (1994) and Zlakine et al., J. Cell Science 110: 673-679 (1997), and can be incorporated in chimeric molecules to induce membrane localization. In certain embodiments, a native sequence from a protein containing an acylation motif is incorporated into a chimeric protein. For example, in some embodiments, an N-terminal portion of Lck, Fyn or Yes or a G-protein alpha subunit, such as the first twenty-five N-terminal amino acids or fewer from such proteins (e.g., about 5 to about 20 amino acids, about 10 to about 19 amino acids, or about 15 to about 19 amino acids of the native sequence with optional mutations), may be incorporated within the N-terminus of a chimeric protein. In certain embodiments, a C-terminal sequence of about 25 amino acids or less from a G-protein gamma subunit containing a CAAX box motif sequence (e.g., about 5 to about 20 amino acids, about 10 to about 18 amino acids, or about 15 to about 18 amino acids of the native sequence with optional mutations) can be linked to the C-terminus of a chimeric protein.

In some embodiments, an acyl moiety has a log p value of +1 to +6, and sometimes has a log p value of +3 to +4.5. Log p values are a measure of hydrophobicity and often are derived from octanol/water partitioning studies, in which molecules with higher hydrophobicity partition into octanol with higher frequency and are characterized as having a higher log p value. Log p values are published for a number of lipophilic molecules and log p values can be calculated using known partitioning processes (e.g., Chemical Reviews, Vol. 71, Issue 6, page 599, where entry 4493 shows lauric acid having a log p value of 4.2). Any acyl moiety can be linked to a peptide composition discussed above and tested for antimicrobial activity using known methods and those discussed hereafter. The acyl moiety sometimes is a C1-C20 alkyl, C2-C20 alkenyl, C2-C20 alkynyl, C3-C6 cycloalkyl, C1-C4 haloalkyl, C4-C12 cycloalkylalkyl, aryl, substituted aryl, or aryl (C1-C4) alkyl, for example. Any acyl-containing moiety sometimes is a fatty acid, and examples of fatty acid moieties are propyl (C3), butyl (C4), pentyl (C5), hexyl (C6), heptyl (C7), octyl (C8), nonyl (C9), decyl (C10), undecyl (C11), lauryl (C12), myristyl (C14), palmityl (C16), stearyl (C18), arachidyl (C20), behenyl (C22) and lignoceryl moieties (C24), and each moiety can contain 0, 1, 2, 3, 4, 5, 6, 7 or 8 unsaturations (i.e., double bonds). An acyl moiety sometimes is a lipid molecule, such as a phosphatidyl lipid (e.g., phosphatidyl serine, phosphatidyl inositol, phosphatidyl ethanolamine, phosphatidyl choline), sphingolipid (e.g., shingomyelin, sphingosine, ceramide, ganglioside, cerebroside), or modified versions thereof. In certain embodiments, one, two, three, four or five or more acyl moieties are linked to a membrane association region. A chimeric protein herein also may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFbeta, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

Any membrane-targeting sequence can be employed that is functional in the host and may, or may not, be associated with one of the other domains of the chimeric protein. In some embodiments, such sequences include, but are not limited to myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster J P et al, Biology of the Cell (2007) 99, 1-12, Vincent, S., et al., Nature Biotechnology 21:936-40, 1098 (2003).

Additional protein domains exist that can increase protein retention at various membranes. For example, an ˜120 amino acid pleckstrin homology (PH) domain is found in over 200 human proteins that are typically involved in intracellular signaling. PH domains can bind various phosphatidylinositol (PI) lipids within membranes (e.g. PI (3, 4, 5)-P3, PI (3,4)-P2, PI (4,5)-P2) and thus play a key role in recruiting proteins to different membrane or cellular compartments. Often the phosphorylation state of PI lipids is regulated, such as by PI-3 kinase or PTEN, and thus, interaction of membranes with PH domains are not as stable as by acyl lipids.

AP1903 for Injection

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (˜10 mg AP1903 for Injection total per vial).

AP1903 is removed from the refrigerator the night before the patient is dosed and stored at a temperature of approximately 21° C. overnight, so that the solution is clear prior to dilution. The solution is prepared within 30 minutes of the start of the infusion in glass or polyethylene bottles or non-DEHP bags and stored at approximately 21° C. prior to dosing.

All study medication is maintained at a temperature between 2 degrees C. and 8 degrees C., protected from excessive light and heat, and stored in a locked area with restricted access.

Upon determining a need to administer AP1903 and induce the inducible Caspase-9 polypeptide, patients may be, for example, administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not to be recalculated unless body weight fluctuates by ≥10%. The calculated dose is diluted in 100 mL in 0.9% normal saline before infusion.

In a previous Phase 1 study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cₘₐₓ values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. AP1903 for Injection was shown to be safe and well tolerated at all dose levels and demonstrated a favorable pharmacokinetic profile. Iuliucci J D, et al., J Clin Pharmacol. 41: 870-9, 2001.

The fixed dose of AP1903 for injection used, for example, may be 0.4 mg/kg intravenously infused over 2 hours. The amount of AP1903 needed in vitro for effective signaling of cells is 10-100 nM (1600 Da MW). This equates to 16-160 μg/L or -0.016-1.6 mg/kg (1.6-160 μg/kg). Doses up to 1 mg/kg were well-tolerated in the Phase 1 study of AP1903 discussed above. Therefore, 0.4 mg/kg may be a safe and effective dose of AP1903 for this Phase I study in combination with the therapeutic cells.

### Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus-I thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, beta-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, an scFv, TCR, or other antigen recognition moiety that binds to CD19. In some embodiments, a polypeptide may be included in the expression vector to aid in sorting cells. For example, the CD34 minimal epitope may be incorporated into the vector. In some embodiments, the expression vectors used to express the chimeric antigen receptors or chimeric stimulating molecules provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

Transmembrane Regions

A chimeric antigen receptor herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3ζ, CD4, CD5, CD8, CD8a, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. The chimeric antigen receptor may further comprise a region of amino acids between the transmembrane domain and the cytoplasmic domain, which are naturally associated with the polypeptide from which the transmembrane domain is derived.

Control Regions

### Promoters

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example, in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene products are toxic (add in more inducible promoters).

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli, he tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

In other examples, promoters may be selected that are developmentally regulated and are active in particular differentiated cells. Thus, for example, a promoter may not be active in a pluripotent stem cell, but, for example, where the pluripotent stem cell differentiates into a more mature cell, the promoter may then be activated.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity (Bojak, A., et al., 2002. Vaccine. 20:1975-79; Cazeaux., N., et al., 2002. Vaccine 20:3322-31). For example, tissue specific promoters such as the PSA associated promoter or prostate-specific glandular kallikrein, or the muscle creatine kinase gene may be used where appropriate.

Examples of tissue specific or differentiation specific promoters include, but are not limited to, the following: B29 (B cells); CD14 (monocytic cells); CD43 (leukocytes and platelets); CD45 (hematopoietic cells); CD68 (macrophages); desmin (muscle); elastase-1 (pancreatic acinar cells); endoglin (endothelial cells); fibronectin (differentiating cells, healing tissues); and Flt-1 (endothelial cells); GFAP (astrocytes).

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8, 42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

### Enhancers

Enhancers are genetic elements that increase transcription from a promoter located at a distant position on the same molecule of DNA. Early examples include the enhancers associated with immunoglobulin and T cell receptors that both flank the coding sequence and occur within several introns. Many viral promoters, such as CMV, SV40, and retroviral LTRs are closely associated with enhancer activity and are often treated like single elements. Enhancers are organized much like promoters. That is, they are composed of many individual elements, each of which binds to one or more transcriptional proteins. The basic distinction between enhancers and promoters is operational. An enhancer region as a whole stimulates transcription at a distance and often independent of orientation; this need not be true of a promoter region or its component elements. On the other hand, a promoter has one or more elements that direct initiation of RNA synthesis at a particular site and in a particular orientation, whereas enhancers lack these specificities. Promoters and enhancers are often overlapping and contiguous, often seeming to have a very similar modular organization. A subset of enhancers is locus-control regions (LCRs) that can not only increase transcriptional activity, but (along with insulator elements) can also help to insulate the transcriptional element from adjacent sequences when integrated into the genome. Any promoter/enhancer combination (as per the Eukaryotic Promoter Data Base EPDB) can be used to drive expression of the gene, although many will restrict expression to a particular tissue type or subset of tissues (reviewed in, for example, Kutzler, M. A., and Weiner, D. B., 2008. Nature Reviews Genetics 9:776-88). Examples include, but are not limited to, enhancers from the human actin, myosin, hemoglobin, muscle creatine kinase, sequences, and from viruses CMV, RSV, and EBV. Appropriate enhancers may be selected for particular applications. Eukaryotic cells can support cytoplasmic transcription from certain bacterial promoters if the appropriate bacterial polymerase is provided, either as part of the delivery complex or as an additional genetic expression construct.

### Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the present methods, and any such sequence is employed such as human or bovine growth hormone and SV40 polyadenylation signals and LTR polyadenylation signals. One non-limiting example is the SV40 polyadenylation signal present in the pCEP3 plasmid (Invitrogen, Carlsbad, Calif.). Also, contemplated as an element of the expression cassette is a terminator. These elements can serve to enhance message levels and to minimize read through from the cassette into other sequences. Termination or poly(A) signal sequences may be, for example, positioned about 11-30 nucleotides downstream from a conserved sequence (AAUAAA) at the 3′ end of the mRNA (Montgomery, D. L., et al., 1993. DNA Cell Biol. 12:777-83; Kutzler, M. A., and Weiner, D. B., 2008. Nature Rev. Gen. 9:776-88).

4. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. The initiation codon is placed in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements is used to create multigene, or polycistronic messages. IRES elements are able to bypass the ribosome-scanning model of 5′ methylated cap-dependent translation and begin translation at internal sites (Pelletier and Sonenberg, Nature, 334:320-325, 1988). IRES elements from two members of the picornavirus family (polio and encephalomyocarditis) have been discussed (Pelletier and Sonenberg, 1988), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### Sequence Optimization

Protein production may also be increased by optimizing the codons in the transgene. Species specific codon changes may be used to increase protein production. Also, codons may be optimized to produce an optimized RNA, which may result in more efficient translation. By optimizing the codons to be incorporated in the RNA, elements such as those that result in a secondary structure that causes instability, secondary mRNA structures that can, for example, inhibit ribosomal binding, or cryptic sequences that can inhibit nuclear export of mRNA can be removed (Kutzler, M. A., and Weiner, D. B., 2008. Nature Rev. Gen. 9:776-88; Yan, J. et al., 2007. Mol. Ther. 15:411-21; Cheung, Y. K., et al., 2004. Vaccine 23:629-38; Narum., D. L., et al., 2001. 69:7250-55; Yadava, A., and Ockenhouse, C. F., 2003. Infect. Immun. 71:4962-69; Smith., J. M., et al., 2004. AIDS Res. Hum. Retroviruses 20:1335-47; Zhou, W., et al., 2002. Vet. Microbiol. 88:127-51; Wu, X., et al., 2004. Biochem. Biophys. Res. Commun. 313:89-96; Zhang, W., et al., 2006. Biochem. Biophys. Res. Commun. 349:69-78; Deml, L. A., et al., 2001. J. Virol. 75:1099-11001; Schneider, R. M., et al., 1997. J. Virol. 71:4892-4903; Wang, S. D., et al., 2006. Vaccine 24:4531-40; zur Megede, J., et al., 2000. J. Virol. 74:2628-2635). For example, the FBP12, the Caspase polypeptide, and the CD19 sequences may be optimized by changes in the codons.

### Leader Sequences

Leader sequences may be added to enhance the stability of mRNA and result in more efficient translation. The leader sequence is usually involved in targeting the mRNA to the endoplasmic reticulum. Examples include the signal sequence for the HIV-1 envelope glycoprotein (Env), which delays its own cleavage, and the IgE gene leader sequence (Kutzler, M. A., and Weiner, D. B., 2008. Nature Rev. Gen. 9:776-88; Li, V., et al., 2000. Virology 272:417-28; Xu, Z. L., et al. 2001. Gene 272:149-56; Malin, A. S., et al., 2000. Microbes Infect. 2:1677-85; Kutzler, M. A., et al., 2005. J. Immunol. 175:112-125; Yang, J. S., et al., 2002. Emerg. Infect. Dis. 8:1379-84; Kumar., S., et al., 2006. DNA Cell Biol. 25:383-92; Wang, S., et al., 2006. Vaccine 24:4531-40). The IgE leader may be used to enhance insertion into the endoplasmic reticulum (Tepler, I, et al. (1989) J. Biol. Chem. 264:5912).

Expression of the transgenes may be optimized and/or controlled by the selection of appropriate methods for optimizing expression. These methods include, for example, optimizing promoters, delivery methods, and gene sequences, (for example, as presented in Laddy, D. J., et al., 2008. PLoS. ONE 3 e2517; Kutzler, M. A., and Weiner, D. B., 2008. Nature Rev. Gen. 9:776-88).

### Nucleic Acids

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides.

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are often used for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. The low or high stringency conditions may be further modified to suit a particular application.

### Nucleic Acid Modification

Any of the modifications discussed below may be applied to a nucleic acid. Examples of modifications include alterations to the RNA or DNA backbone, sugar or base, and various combinations thereof. Any suitable number of backbone linkages, sugars and/or bases in a nucleic acid can be modified (e.g., independently about 5%, 10%, 15%, 20%, 25%, 30%, 35%, 40%, 45%, 50%, 55%, 60%, 65%, 70%, 75%, 80%, 85%, 90%, 95%, up to 100%). An unmodified nucleoside is any one of the bases adenine, cytosine, guanine, thymine, or uracil joined to the 1′ carbon of beta-D-ribo-furanose.

A modified base is a nucleotide base other than adenine, guanine, cytosine and uracil at a 1′ position. Non-limiting examples of modified bases include inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e. g., 5-methylcytidine), 5-alkyluridines (e. g., ribothymidine), 5-halouridine (e. g., 5-bromouridine) or 6-azapyrimidines or 6-alkylpyrimidines (e. g. 6-methyluridine), propyne, and the like. Other non-limiting examples of modified bases include nitropyrrolyl (e.g., 3-nitropyrrolyl), nitroindolyl (e.g., 4-, 5-, 6-nitroindolyl), hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, difluorotolyl, 4-fluoro-6-methylbenzimidazole, 4-methylbenzimidazole, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl and the like.

In some embodiments, for example, a nucleic acid may comprise modified nucleic acid molecules, with phosphate backbone modifications. Non-limiting examples of backbone modifications include phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl modifications. In certain instances, a ribose sugar moiety that naturally occurs in a nucleoside is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. The hexose may be a D-hexose, glucose, or mannose. In certain instances, the polycyclic heteroalkyl group may be a bicyclic ring containing one oxygen atom in the ring.

In certain instances, the polycyclic heteroalkyl group is a bicyclo[2.2.1]heptane, a bicyclo[3.2.1]octane, or a bicyclo[3.3.1]nonane.

Nitropyrrolyl and nitroindolyl nucleobases are members of a class of compounds known as universal bases. Universal bases are those compounds that can replace any of the four naturally occurring bases without substantially affecting the melting behavior or activity of the oligonucleotide duplex. In contrast to the stabilizing, hydrogen-bonding interactions associated with naturally occurring nucleobases, oligonucleotide duplexes containing 3-nitropyrrolyl nucleobases may be stabilized solely by stacking interactions. The absence of significant hydrogen-bonding interactions with nitropyrrolyl nucleobases obviates the specificity for a specific complementary base. In addition, 4-, 5- and 6-nitroindolyl display very little specificity for the four natural bases. Procedures for the preparation of 1-(2′-O-methyl-.beta.-D-ribofuranosyl)-5-nitroindole are discussed in Gaubert, G.; Wengel, J. Tetrahedron Letters 2004, 45, 5629. Other universal bases include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof.

Difluorotolyl is a non-natural nucleobase that functions as a universal base. Difluorotolyl is an isostere of the natural nucleobase thymine. But unlike thymine, difluorotolyl shows no appreciable selectivity for any of the natural bases. Other aromatic compounds that function as universal bases are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-di methyl indolyl, phenyl, napthalenyl, anthracenyl, phenanthracenyl, pyrenyl, stilbenyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above, see: Schweitzer et al., J. Org. Chem., 59:7238-7242 (1994);

In addition, chemical substituents, for example cross-linking agents, may be used to add further stability or irreversibility to the reaction. Non-limiting examples of cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl) dithio]propioimidate.

A nucleotide analog may also include a “locked” nucleic acid. Certain compositions can be used to essentially “anchor” or “lock” an endogenous nucleic acid into a particular structure. Anchoring sequences serve to prevent disassociation of a nucleic acid complex, and thus not only can prevent copying but may also enable labeling, modification, and/or cloning of the endogeneous sequence. The locked structure may regulate gene expression (i.e. inhibit or enhance transcription or replication), or can be used as a stable structure that can be used to label or otherwise modify the endogenous nucleic acid sequence, or can be used to isolate the endogenous sequence, i.e. for cloning.

Nucleic acid molecules need not be limited to those molecules containing only RNA or DNA, but further encompass chemically-modified nucleotides and non-nucleotides. The percent of non-nucleotides or modified nucleotides may be from 1% to 100% (e.g., about 5, 10, 15, 20, 25, 30, 35, 40, 45, 50, 55, 60, 65, 70, 75, 80, 85, 90 or 95%).

### Nucleic Acid Preparation

In some embodiments, a nucleic acid is provided for use as a control or standard in an assay, or therapeutic, for example. A nucleic acid may be made by any technique known in the art, such as for example, chemical synthesis, enzymatic production or biological production. Nucleic acids may be recovered or isolated from a biological sample. The nucleic acid may be recombinant or it may be natural or endogenous to the cell (produced from the cell's genome). It is contemplated that a biological sample may be treated in a way so as to enhance the recovery of small nucleic acid molecules. Generally, methods may involve lysing cells with a solution having guanidinium and a detergent.

Nucleic acid synthesis may also be performed according to standard methods. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques or via deoxynucleoside H-phosphonate intermediates. Various different mechanisms of oligonucleotide synthesis have been disclosed elsewhere.

Nucleic acids may be isolated using known techniques. In particular embodiments, methods for isolating small nucleic acid molecules, and/or isolating RNA molecules can be employed. Chromatography is a process used to separate or isolate nucleic acids from protein or from other nucleic acids. Such methods can involve electrophoresis with a gel matrix, filter columns, alcohol precipitation, and/or other chromatography. If a nucleic acid from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

Methods may involve the use of organic solvents and/or alcohol to isolate nucleic acids. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column is effective for such isolation procedures.

A nucleic acid isolation processes may sometimes include: a) lysing cells in the sample with a lysing solution comprising guanidinium, where a lysate with a concentration of at least about 1 M guanidinium is produced; b) extracting nucleic acid molecules from the lysate with an extraction solution comprising phenol; c) adding to the lysate an alcohol solution to form a lysate/alcohol mixture, wherein the concentration of alcohol in the mixture is between about 35% to about 70%; d) applying the lysate/alcohol mixture to a solid support; e) eluting the nucleic acid molecules from the solid support with an ionic solution; and, f) capturing the nucleic acid molecules. The sample may be dried down and resuspended in a liquid and volume appropriate for subsequent manipulation.

Provided herein are compositions or kits that comprise nucleic acid comprising the polynucleotides of the present application. Thus, compositions or kits may, for example, comprise both the first and second polynucleotides, encoding the first and second chimeric polypeptides. The nucleic acid may comprise more than one nucleic acid species, that is, for example, the first nucleic acid species comprises the first polynucleotide, and the second nucleic acid species comprises the second polynucleotide. In other examples, the nucleic acid may comprise both the first and second polynucleotides. The kit may, in addition, comprise the first or second ligand, or both. The kits may, in some embodiments, provide a nucleic acid composition, such as, for example, a virus, for example, a retrovirus, that comprises at least two polynucleotides, wherein the polynucleotides express, for example, an inducible pro-apoptotic polypeptide and a chimeric antigen receptor; an inducible pro-apoptotic polypeptide and a recombinant TCR; an inducible pro-apoptotic polypeptide and a chimeric costimulating polypeptide such as, for example an inducible chimeric MyD88 polypeptide, an inducible chimeric truncated MyD88 polypeptide, and optionally a CD40 polypeptide. The nucleic acid composition may comprise polynucleotides encoding an inducible pro-apoptotic polypeptide, an inducible chimeric MyD88 polypeptide or an inducible chimeric truncated MyD88 polypeptide, and optionally a CD40 polypeptide, and a chimeric antigen receptor or a recombinant T cell receptor.

Thus, in certain embodiments, kits are provided that comprise a nucleic acid composition such as, for example a virus, for example, a retrovirus, that comprises a polynucleotide that encodes 1) an iRC9 or iRmC9 polypeptide and an iM (MyD88FvFv) or iMC polypeptide; 2) an RC9 or iRmC9 polypeptide and a chimeric antigen receptor; 3) an iRC9 or iRmC9 polypeptide and a recombinant TCR; 4) an iC9 polypeptide and an iRMC or iRM (iRMyD88) polypeptide; 5) an iC9 polypeptide and an iRMC or iRM (iRMyD88) polypeptide and a chimeric antigen receptor; or 6) an iC9 polypeptide and an iRMC or iRM (iRMyD88) polypeptide and a recombinant T cell receptor.

### Methods of Gene Transfer

In order to mediate the effect of the transgene expression in a cell, it will be necessary to transfer the expression constructs into a cell. Such transfer may employ viral or non-viral methods of gene transfer. This section provides a discussion of methods and compositions of gene transfer. A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism. A host cell can, and has been, used as a recipient for vectors. Host cells may be derived from prokaryotes or eukaryotes, depending upon whether the desired result is replication of the vector or expression of part or all of the vector-encoded polynucleotide sequences. Numerous cell lines and cultures are available for use as a host cell, and they can be obtained through the American Type Culture Collection (ATCC), which is an organization that serves as an archive for living cultures and genetic materials.

An appropriate host may be determined. Generally, this is based on the vector backbone and the desired result. A plasmid or cosmid, for example, can be introduced into a prokaryote host cell for replication of many vectors. Bacterial cells used as host cells for vector replication and/or expression include DH5alpha, JM109, and KCB, as well as a number of commercially available bacterial hosts such as SURE® Competent Cells and SOLOPACK Gold Cells (STRATAGENE®, La Jolla, Calif.). Alternatively, bacterial cells such as E. coli LE392 could be used as host cells for phage viruses. Eukaryotic cells that can be used as host cells include, but are not limited to yeast, insects and mammals. Examples of mammalian eukaryotic host cells for replication and/or expression of a vector include, but are not limited to, HeLa, NIH3T3, Jurkat, 293, COS, CHO, Saos, and PC12. Examples of yeast strains include, but are not limited to, YPH499, YPH500 and YPH501.

Nucleic acid vaccines may include, for example, non-viral DNA vectors, “naked” DNA and RNA, and viral vectors. Methods of transforming cells with these vaccines, and for optimizing the expression of genes included in these vaccines are known and are also discussed herein.

Examples of Methods of Nucleic Acid or Viral Vector Transfer

Any appropriate method may be used to transfect or transform the cells, or to administer the nucleotide sequences or compositions of the present methods. Certain examples are presented herein, and further include methods such as delivery using cationic polymers, lipid like molecules, and certain commercial products such as, for example, IN-VIVO-JET PEI.

### Ex Vivo Transformation

Various methods are available for transfecting vascular cells and tissues removed from an organism in an ex vivo setting. For example, canine endothelial cells have been genetically altered by retroviral gene transfer in vitro and transplanted into a canine (Wilson et al., Science, 244:1344-1346, 1989). In another example, Yucatan minipig endothelial cells were transfected by retrovirus in vitro and transplanted into an artery using a double-balloon catheter (Nabel et al., Science, 244(4910):1342-1344, 1989). Thus, it is contemplated that cells or tissues may be removed and transfected ex vivo using the polynucleotides presented herein. In particular aspects, the transplanted cells or tissues may be placed into an organism.

### Injection

In certain embodiments, an antigen presenting cell or a nucleic acid or viral vector may be delivered to an organelle, a cell, a tissue or an organism via one or more injections (i.e., a needle injection), such as, for example, subcutaneous, intradermal, intramuscular, intravenous, intraprostatic, intratumor, intraperitoneal, etc. Methods of injection include, for example, injection of a composition comprising a saline solution. Further embodiments include the introduction of a polynucleotide by direct microinjection. The amount of the expression vector used may vary upon the nature of the antigen as well as the organelle, cell, tissue or organism used. Intradermal, intranodal, or intralymphatic injections are some of the more commonly used methods of DC administration. Intradermal injection is characterized by a low rate of absorption into the bloodstream but rapid uptake into the lymphatic system. The presence of large numbers of Langerhans dendritic cells in the dermis will transport intact as well as processed antigen to draining lymph nodes. Proper site preparation is necessary to perform this correctly (i.e., hair is clipped in order to observe proper needle placement). Intranodal injection allows for direct delivery of antigen to lymphoid tissues. Intralymphatic injection allows direct administration of DCs.

### Electroporation

In certain embodiments, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

Transfection of eukaryotic cells using electroporation has been quite successful. Mouse pre-B lymphocytes have been transfected with human kappa-immunoglobulin genes (Potter et al., (1984) Proc. Nat'l Acad. Sci. USA, 81, 7161-7165), and rat hepatocytes have been transfected with the chloramphenicol acetyltransferase gene (Tur-Kaspa et al., (1986) Mol. Cell Biol., 6, 716-718) in this manner.

In vivo electroporation for vaccines, or eVac, is clinically implemented through a simple injection technique. A DNA vector encoding a polypeptide is injected intradermally in a patient. Then electrodes apply electrical pulses to the intradermal space causing the cells localized there, especially resident dermal dendritic cells, to take up the DNA vector and express the encoded polypeptide. These polypeptide-expressing cells activated by local inflammation can then migrate to lymph-nodes, presenting antigens, for example. A nucleic acid is electrophoretically administered when it is administered using electroporation, following, for example, but not limited to, injection of the nucleic acid or any other means of administration where the nucleic acid may be delivered to the cells by electroporation

Methods of electroporation are discussed in, for example, Sardesai, N. Y., and Weiner, D. B., Current Opinion in Immunotherapy 23:421-9 (2011) and Ferraro, B. et al., Human Vaccines 7:120-127 (2011), which are hereby incorporated by reference herein in their entirety.

### Calcium Phosphate

In other embodiments, a polynucleotide is introduced to the cells using calcium phosphate precipitation. Human KB cells have been transfected with adenovirus 5 DNA (Graham and van der Eb, (1973) Virology, 52, 456-467) using this technique. Also in this manner, mouse L(A9), mouse C127, CHO, CV-1, BHK, NIH3T3 and HeLa cells were transfected with a neomycin marker gene (Chen and Okayama, Mol. Cell Biol., 7(8):2745-2752, 1987), and rat hepatocytes were transfected with a variety of marker genes (Rippe et al., Mol. Cell Biol., 10:689-695, 1990).

### DEAE-Dextran

In another embodiment, a polynucleotide is delivered into a cell using DEAE-dextran followed by polyethylene glycol. In this manner, reporter plasmids were introduced into mouse myeloma and erythroleukemia cells (Gopal, T. V., Mol Cell Biol. 1985 May; 5(5):1188-90).

### Sonication Loading

Additional embodiments include the introduction of a polynucleotide by direct sonic loading. LTK-fibroblasts have been transfected with the thymidine kinase gene by sonication loading (Fechheimer et al., (1987) Proc. Nat'l Acad. Sci. USA, 84, 8463-8467).

### Liposome-Mediated Transfection

In a further embodiment, a polynucleotide may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine (Gibco BRL) or Superfect (Qiagen).

### Receptor Mediated Transfection

Still further, a polynucleotide may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity.

Certain receptor-mediated gene targeting vehicles comprise a cell receptor-specific ligand and a polynucleotide-binding agent. Others comprise a cell receptor-specific ligand to which the polynucleotide to be delivered has been operatively attached. Several ligands have been used for receptor-mediated gene transfer (Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085), which establishes the operability of the technique. Specific delivery in the context of another mammalian cell type has been discussed (Wu and Wu, Adv. Drug Delivery Rev., 12:159-167, 1993; incorporated herein by reference). In certain aspects, a ligand is chosen to correspond to a receptor specifically expressed on the target cell population. In other embodiments, a polynucleotide delivery vehicle component of a cell-specific polynucleotide-targeting vehicle may comprise a specific binding ligand in combination with a liposome. The polynucleotide(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a polynucleotide to cells that exhibit upregulation of the EGF receptor.

In still further embodiments, the polynucleotide delivery vehicle component of a targeted delivery vehicle may be a liposome itself, which may, for example, comprise one or more lipids or glycoproteins that direct cell-specific binding. For example, lactosyl-ceramide, a galactose-terminal asialoganglioside, have been incorporated into liposomes and observed an increase in the uptake of the insulin gene by hepatocytes (Nicolau et al., (1987) Methods Enzymol., 149, 157-176). It is contemplated that the tissue-specific transforming constructs may be specifically delivered into a target cell in a similar manner.

Microprojectile Bombardment

Microprojectile bombardment techniques can be used to introduce a polynucleotide into at least one, organelle, cell, tissue or organism (U.S. Pat. Nos. 5,550,318; 5,538,880; 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., (1987) Nature, 327, 70-73). There are a wide variety of microprojectile bombardment techniques known in the art, many of which are applicable to the present methods. In this microprojectile bombardment, one or more particles may be coated with at least one polynucleotide and delivered into cells by a propelling force. Several devices for accelerating small particles have been developed. One such device relies on a high voltage discharge to generate an electrical current, which in turn provides the motive force (Yang et al., (1990) Proc. Nat'l Acad. Sci. USA, 87, 9568-9572). The microprojectiles used have consisted of biologically inert substances such as tungsten or gold particles or beads. Exemplary particles include those comprised of tungsten, platinum, and, in certain examples, gold, including, for example, nanoparticles. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

### Examples of Methods of Viral Vector-Mediated Transfer

Any viral vector suitable for administering nucleotide sequences, or compositions comprising nucleotide sequences, to a cell or to a subject, such that the cell or cells in the subject may express the genes encoded by the nucleotide sequences may be employed in the present methods. In certain embodiments, a transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

### Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1 B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present methods, it is possible to achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol Biol. 1984 Jun. 5; 175(4):493-510). Therefore, inclusion of these elements in an adenoviral vector may permits replication.

In addition, the packaging signal for viral encapsulation is localized between 194-385 bp (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element derives from the packaging function of adenovirus. It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pseudotyped” virus, Ad5f35, has been the basis for several clinically developed viral isolates. Moreover, various biochemical methods exist to modify the fiber to allow re-targeting of the virus to target cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

### Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990). Thus, for example, the present technology includes, for example, cells whereby the polynucleotide used to transduce the cell is integrated into the genome of the cell. In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), and New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248). An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may be desired. A different approach to targeting of recombinant retroviruses was designed, which used biotinylated antibodies against a retroviral envelope protein and against a specific cell receptor. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

### Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins believed to hold AAV structural expression in check, and helper virus infection is thought to remove this block. The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

### Other Viral Vectors

Other viral vectors are employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

### Methods for Treating a Disease

The present methods also encompass methods of treatment or prevention of a disease where administration of cells by, for example, infusion, may be beneficial.

Cells, such as, for example, T cells, tumor infiltrating lymphocytes, natural killer cells, natural killer T cells, or progenitor cells, such as, for example, hematopoietic stem cells, mesenchymal stromal cells, stem cells, pluripotent stem cells, and embryonic stem cells may be used for cell therapy. The cells may be from a donor, or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. Mesenchymal stromal cells have also, for example, been used to provide immunosuppressive activity, and may be used in the treatment of graft versus host disease and autoimmune disorders. The cells provided in the present application contain a safety switch that may be valuable in a situation where following cell therapy, the activity of the therapeutic cells needs to be increased, or decreased. For example, where T cells that express a chimeric antigen receptor are provided to the patient, in some situations there may be an adverse event, such as off-target toxicity. Ceasing the administration of the ligand would return the therapeutic T cells to a non-activated state, remaining at a low, non-toxic, level of expression. Or, for example, the therapeutic cell may work to decrease the tumor cell, or tumor size, and may no longer be needed. In this situation, administration of the ligand may cease, and the therapeutic cells would no longer be activated. If the tumor cells return, or the tumor size increases following the initial therapy, the ligand may be administered again, in order to activate the chimeric antigen receptor-expressing T cells, and re-treat the patient.

By “therapeutic cell” is meant a cell used for cell therapy, that is, a cell administered to a subject to treat or prevent a condition or disease. In such cases, where the cells have a negative effect, the present methods may be used to remove the therapeutic cells through selective apoptosis.

In other examples, T cells are used to treat various diseases and conditions, and as a part of stem cell transplantation. An adverse event that may occur after haploidentical T cell transplantation is graft versus host disease (GvHD). The likelihood of GvHD occurring increases with the increased number of T cells that are transplanted. This limits the number of T cells that may be infused. By having the ability to selectively remove the infused T cells in the event of GvHD in the patient, a greater number of T cells may be infused, increasing the number to greater than 10⁶, greater than 10⁷, greater than 10⁸, or greater than 10⁹ cells. The number of T cells/kg body weight that may be administered may be, for example, from about 1×10⁴ T cells/kg body weight to about 9×10⁷ T cells/kg body weight, for example about 1, 2, 3, 4, 5, 6, 7, 8, or 9×10⁴; about 1, 2, 3, 4, 5, 6, 7, 8, or 9×10⁵; about 1, 2, 3, 4, 5, 6, 7, 8, or 9×10⁶; or about 1, 2, 3, 4, 5, 6, 7, 8, or 9×10⁷ T cells/kg body weight. In other examples, therapeutic cells other than T cells may be used. The number of therapeutic cells/kg body weight that may be administered may be, for example, from about 1×10⁴ T cells/kg body weight to about 9×10⁷ T cells/kg body weight, for example about 1, 2, 3, 4, 5, 6, 7, 8, or 9×10⁴; about 1, 2, 3, 4, 5, 6, 7, 8, or 9×10⁶; about 1, 2, 3, 4, 5, 6, 7, 8, or 9×10⁶; or about 1, 2, 3, 4, 5, 6, 7, 8, or 9×10⁷ therapeutic cells/kg body weight.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition, such as the multimeric ligand presented herein, would be the amount that achieves this selected result of selectively removing the cells that include the Caspase-9 vector, such that over 60%, 70%, 80%, 85%, 90%, 95%, or 97% of the Caspase-9 expressing cells are killed. The term is also synonymous with “sufficient amount.” The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The terms “contacted” and “exposed,” when applied to a cell, tissue or organism, are used herein to discuss the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

### Optimized and Personalized Therapeutic Treatment

The induction of apoptosis after administration of the dimer may be optimized by determining the stage of graft versus host disease, or the number of undesired therapeutic cells that remain in the patient.

For example, determining that a patient has GvHD, and the stage of the GvHD, provides an indication to a clinician that it may be necessary to induce Caspase-9 associated apoptosis by administering the multimeric ligand. In another example, determining that a patient has a reduced level of GvHD after treatment with the multimeric ligand may indicate to the clinician that no additional dose of the multimeric ligand is needed. Similarly, after treatment with the multimeric ligand, determining that the patient continues to exhibit GvHD symptoms, or suffers a relapse of GvHD may indicate to the clinician that it may be necessary to administer at least one additional dose of multimeric ligand. The term “dosage” is meant to include both the amount of the dose and the frequency of administration, such as, for example, the timing of the next dose.

In other embodiments, following administration of therapeutic cells, for example, therapeutic cells which express a chimeric antigen receptor in addition to the inducible Caspase-9 polypeptide, in the event of a need to reduce the number of modified cells or in vivo modified cells, the multimeric ligand may be administered to the patient. In these embodiments, the methods comprise determining the presence or absence of a negative symptom or condition, such as Graft vs Host Disease, or off target toxicity, and administering a dose of the multimeric ligand. The methods may further comprise monitoring the symptom or condition and administering an additional dose of the multimeric ligand in the event the symptom or condition persists. This monitoring and treatment schedule may continue while the therapeutic cells that express chimeric antigen receptors or chimeric signaling molecules remain in the patient.

An indication of adjusting or maintaining a subsequent drug dose, such as, for example, a subsequence dose of the multimeric ligand, and/or the subsequent drug dosage, can be provided in any convenient manner. An indication may be provided in tabular form (e.g., in a physical or electronic medium) in some embodiments. For example, the graft versus host disease observed symptoms may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms or the GvHD stage is provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

In some examples, a dose, or multiple doses of the ligand may be administered before clinical manifestations of GvHD, or other symptoms, such as CRS symptoms, are apparent. In this example, cell therapy is terminated before the appearance of negative symptoms. In other embodiments, such as, for example, hematopoietic cell transplant for the treatment of a genetic disease, the therapy may be terminated after the transplant has made progress toward engraftment, but before clinically observable GvHD, or other negative symptoms, can occur. In other examples, the ligand may be administered to eliminate the modified cells in order to eliminate on target/off-tumor cells, such as, for example, healthy B cells co-expressing the B cell-associated target antigen.

Methods as presented herein include without limitation the delivery of an effective amount of an activated cell, a nucleic acid or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

### Dual Control of Therapeutic Cells and Heterodimerizer Control of Apoptosis for Controlled Therapy

Nucleic acids and cells provided herein may be used to achieve dual control of therapeutic cells for controlled therapy. For example, the subject may be diagnosed with a condition, such as a tumor, where there is a need to deliver targeted chimeric antigen receptor therapy. Methods discussed herein provide several examples of ways to control therapy in order to induce activity of the CAR-expressing therapeutic cells, and also to provide a safety switch should there be a need to discontinue therapy completely, or to reduce the number or percent of the therapeutic cells in the subject.

In certain examples, modified T cells are administered to a subject that express the following polypeptides: 1. A chimeric polypeptide (iMyD88/CD40, or “iMC”) that comprises two or more FKBP12 ligand binding regions and a costimulatory polypeptide or polypeptides, such as, for example, MyD88 or truncated MyD88 and CD40; 2. A chimeric proapoptotic polypeptide that comprises one or more FRB ligand binding regions and a Caspase-9 polypeptide; 3. A chimeric antigen receptor polypeptide comprising an antigen recognition moiety that binds to a target antigen. In this example, the target antigen is a tumor antigen present on tumor cells in the subject. Following administration, the ligand AP1903 may be administered to the subject, which induces iMC activation of the CAR-T cell. The therapy is monitored, for example, the tumor size or growth may be assessed during the course of therapy. One or more doses of the ligand may be administered during the course of therapy.

Therapy may be modulated by discontinuing administration of AP1903, which may lower the activation level of the CAR-T cell. To discontinue CAR-T cell therapy, the safety switch—chimeric Caspase-9 polypeptide may be activated by administering a rapalog, which binds to the FRB ligand binding region. The amount and dosing schedule of the rapalog may be determined based on the level of CAR-T cell therapy that is needed. As a safety switch, the dose of the rapalog is an amount effective to remove at least 90%, 95%, 97%, 98%, or 99% of the administered modified cells. In other examples, the dose is an amount effective to remove up to 30%, 40%, 50%, 60%, 70%, 80%, 90, 95%, or 100% of the cells that express the chimeric caspase polypeptide, if there is a need to reduce the level of CAR-T cell therapy, but not completely stop the therapy. This may be measured, for example, by obtaining a sample from the subject before inducing the safety switch, before administering the rapamycin or rapalog, and obtaining a sample following administration of the rapamycin or rapalog, at, for example 0.5, 1, 2, 3, 4, 5, 6, 7, 8, 9, 10 hours, or 1, 2, 3, 4, 5 days following administration, and comparing the number or concentration of chimeric caspase-expressing cells between the two samples by, for example, any method available, including, for example, detecting the presence of a marker. This method of determining percent removal of the cells may also be used where the inducing ligand is AP1903 or binds to the FKBP12 or FKBP12 variant multimerizing region.

In some examples, the inducible MyD88/CD40 chimeric polypeptide also comprises the chimeric antigen receptor. In these examples, where the two polypeptides are present on the same molecule, the chimeric polypeptide may comprise one or more ligand binding regions.

Chemical Induction of protein Dimerization (CID) has been effectively applied to make cellular suicide or apoptosis inducible with the small molecule homodimerizing ligand, rimiducid (AP1903). This technology underlies the “safety switch” incorporated as a gene therapy adjunct in cell transplants (1, 2). The central tenet of the technology is that normal cellular regulatory pathways that rely on protein-protein interaction as part of a signaling pathway can be adapted to ligand-dependent, conditional control if a small molecule dimerizing drug is used to control the protein-protein oligomerization event (3-5). Induced dimerization of a fusion protein comprising Caspase-9 and FKBP12 or an FKBP12 variant (i.e., “iCaspase9/iCasp9/iC9) using a homodimerizing ligand, such as rimiducid, AP1510 or AP20187, can rapidly effect cell death. Caspase-9 is an initiating caspase that acts as a “gate-keeper” of the apoptotic process (6). Normally, pro-apoptotic molecules (e.g., cytochrome c) released from the mitochondria of apoptotic cells alter the conformation of Apaf-1, a caspase-9-binding scaffold, leading to its oligomerization and formation of the “apoptosome”. This alteration facilitates caspase-9 dimerization and cleavage of its latent form into an active molecule that, in turn, cleaves the “downstream” apoptosis effector, caspase-3, leading to irreversible cell death. Rimiducid binds directly with two FKBP12-V36 moieties and can direct the dimerization of fusion proteins that include FKBP12-V36 (1, 2). iC9 engagement with rimiducid circumvents the need for Apaf1 conversion to the active apoptosome. In this example, the fusion of caspase-9 to protein moieties that engage a heterodimerizing ligand is assayed for its ability to direct its activation and cell death with similar efficacy to rimiducid-mediated iC9 activation.

MyD88 and CD40 were chosen as the basis of the iMC activation switch. MyD88 plays a central signaling role in the detection of pathogens or cell injury by antigen-presenting cells (APCs), like dendritic cells (DCs). Following exposure to pathogen- or necrotic cells-derived “danger” molecules”, a subclass of “pattern recognition receptors”, called Toll-Like Receptors (TLRs) are activated, leading to the aggregation and activation of adapter molecule, MyD88, via homologous TLR-IL1RA (TIR) domains on both proteins. MyD88, in turn, activates downstream signaling, via the rest of the protein. This leads to the upregulation of costimulatory proteins, like CD40, and other proteins, like MHC and proteases, needed for antigen processing and presentation. The fusion of signaling domains from MyD88 and CD40 with two Fv domains, provides iMC (also MC.FvFv), which potently activated DCs following exposure to rimiducid (7). It was later found that iMC is a potent costimulatory protein for T cells, as well.

Rapamycin is a natural product macrolide that binds with high affinity (<1 nM) to FKBP12 and together initiates the high-affinity, inhibitory interaction with the FKBP-Rapamycin-Binding (FRB) domain of mTOR (8). FRB is small (89 amino acids) and can thereby be used as a protein “tag” or “handle” when appended to many proteins (9-11). Coexpression of a FRB-fused protein with a FKBP12-fused protein renders their approximation rapamycin-inducible (12-16). This and the examples that follow provide experiments and results designed to test whether expression of Caspase-9 bound with FKBP and FRB in tandem can also direct apoptosis and serve as the basis for a cell safety switch regulated by the orally available ligand, rapamycin. Further, an inducible MyD88/CD40 rapamycin-sensitive costimulatory polypeptide was developed by fusing FKBP and FRB in tandem with the MyD88/CD40 polypeptide. For this tandem fusion of FKBP and FRB, derivatives of rapamycin (rapalogs) may also be used that do not inhibit mTOR at a low, therapeutic dose. For example, rapamycin, or these rapamycin analogs may bind with selected, MC-FKBP-fused mutant FRB domains, using a heterodimerize to homodimerize two MC-FKBP-FRB polypeptides.

The following references are referred to in this section, and are hereby incorporated by reference herein in their entireties.
1. Straathof K C, Pule M A, Yotnda P, Dotti G, Vanin E F, Brenner M K, Heslop H E, Spencer D M, and Rooney C M. An inducible caspase 9 safety switch for T-cell therapy. Blood. 2005; 105(11):4247-54.
2. Fan L, Freeman K W, Khan T, Pham E, and Spencer D M. Improved artificial death switches based on caspases and FADD. Hum Gene Ther. 1999; 10(14):2273-85.
3. Spencer D M, Wandless T J, Schreiber S L, and Crabtree G R. Controlling signal transduction with synthetic ligands. Science. 1993; 262(5136):1019-24.
4. Acevedo V D, Gangula R D, Freeman K W, Li R, Zhang Y, Wang F, Ayala G E, Peterson L E, Ittmann M, and Spencer D M. Inducible FGFR-1 activation leads to irreversible prostate adenocarcinoma and an epithelial-to-mesenchymal transition. Cancer Cell. 2007; 12(6):559-71.
5. Spencer D M, Belshaw P J, Chen L, Ho S N, Randazzo F, Crabtree G R, and Schreiber S L. Functional analysis of Fas signaling in vivo using synthetic inducers of dimerization. Curr Biol. 1996; 6(7):839-47.
6. Strasser A, Cory S, and Adams J M. Deciphering the rules of programmed cell death to improve therapy of cancer and other diseases. EMBO J. 2011; 30(18):3667-83.
7. Narayanan P, Lapteva N, Seethammagari M, Levitt J M, Slawin K M, and Spencer D M. A composite MyD88/CD40 switch synergistically activates mouse and human dendritic cells for enhanced antitumor efficacy. J Clin Invest. 2011; 121(4):1524-34.
8. Sabatini D M, Erdjument-Bromage H, Lui M, Tempst P, and Snyder S H. RAFT1: a mammalian protein that binds to FKBP12 in a rapamycin-dependent fashion and is homologous to yeast TORs. Cell. 1994; 78(1):35-43.
9. Brown E J, Albers M W, Shin T B, Ichikawa K, Keith C T, Lane W S, and Schreiber S L. A mammalian protein targeted by G1-arresting rapamycin-receptor complex. Nature. 1994; 369(6483):756-8.
10. Chen J, Zheng X F, Brown E J, and Schreiber S L. Identification of an 11-kDa FKBP12-rapamycin-binding domain within the 289-kDa FKBP12-rapamycin-associated protein and characterization of a critical serine residue. Proc Natl Acad Sci USA. 1995; 92(11):4947-51.
11. Choi J, Chen J, Schreiber S L, and Clardy J. Structure of the FKBP12-rapamycin complex interacting with the binding domain of human FRAP. Science. 1996; 273(5272):239-42.
12. Ho S N, Biggar S R, Spencer D M, Schreiber S L, and Crabtree G R. Dimeric ligands define a role for transcriptional activation domains in reinitiation. Nature. 1996; 382(6594):822-6.
13. Klemm J D, Beals C R, and Crabtree G R. Rapid targeting of nuclear proteins to the cytoplasm. Curr Biol. 1997; 7(9):638-44.
14. Bayle J H, Grimley J S, Stankunas K, Gestwicki J E, Wandless T J, and Crabtree G R. Rapamycin analogs with differential binding specificity permit orthogonal control of protein activity. Chem Biol. 2006; 13(1):99-107.
15. Stankunas K, Bayle J H, Gestwicki J E, Lin Y M, Wandless T J, and Crabtree G R. Conditional protein alleles using knockin mice and a chemical inducer of dimerization. Mol Cell. 2003; 12(6):1615-24.
16. Stankunas K, Bayle J H, Havranek J J, Wandless T J, Baker D, Crabtree G R, and Gestwicki J E. Rescue of Degradation-Prone Mutants of the FK506-Rapamycin Binding (FRB) Protein with Chemical Ligands. Chembiochem. 2007.

### Dual-Switch, Chimeric Pro-Apoptotic Polypeptides

The activity of chimeric polypeptides FRB.FKBP_{V}.ΔC9 (dual-control), FKBPᵥ.ΔC9, and or FRB.FKBP.ΔC9 were assayed in response to either the heterodimer, rapamycin, or the homodimer, rimiducid.

Chemical Induction of Dimerization (CID) with small molecules is an effective technology used to generate switches of protein function to alter cell physiology. Rimiducid or AP1903 is a highly specific and efficient dimerizer composed of two identical protein-binding surfaces (based on FK506) arranged tail-to-tail, each with high affinity and specificity for an FKBP mutant, FKBP12v36 or FKBPᵥ. FKBP12v36 is a modified version of FKBP12, in which phenylalanine 36, is replaced with the smaller hydrophobic residue, valine, which accommodates the bulky modification on the FKBP12-binding site of AP1903 [1]. This change increases binding of AP1903 to FKBP12v36 (˜0.1 nM), while binding of AP1903 to native FKBP12 is reduced around 100-fold relative to FK506 [1, 2]. Attachment of one or more Fv domains onto one or more cell signaling molecules that normally rely on homodimerization can convert that protein to rimiducid-induced signaling control. Homodimerization with rimiducid is the basis of both the inducible Caspase-9 (iCaspase-9) “safety switch” and the inducible MyD88/CD40 (iMC) “activation switch” for cellular therapy.

Rapamycin binds to FKBP12, but unlike rimiducid, rapamycin also binds to the FKBP12-Rapamycin-Binding (FRB) domain of mTOR and can induce heterodimerization of signaling domains that are fused to FKBP12 with fusions containing FRB. Expression of Caspase-9 fused with FKBP and FRB in tandem (in both orientations: FKBP.FRB.ΔC9 or FRB.FKBP.ΔC9) can direct apoptosis and serve as the basis for a cell safety switch regulated by the orally available ligand, rapamycin. Importantly, since rimiducid contains a bulky modification on the FKBP12-binding site, this dimerizer is not able to bind to wild type FKBP12.

The FRB.FKBP_{V}.ΔC9 switch provides the option to activate caspase-9 with either rimiducid or rapamycin by mutating the FKBP domain to FKBPv. This flexibility in terms of choice of activating drug may be important in a clinical setting where the clinician can choose to administer the drug based on its specific pharmacological properties. Additionally, this switch provides a molecule to allow for direct comparison between the drug-activating kinetics of rimiducid and rapamycin where the effector is contained within a single molecule.
1. D. Spencer, et al., Science, vol. 262, pp. 1019-1024, 1993.
2. T. Clackson, et al., Proc Natl Acad Sci USA, vol. 95, pp. 10437-10442, 1998.

### Formulations and Routes for Administration to Patients

Where clinical applications are contemplated, it will be necessary to prepare pharmaceutical compositions—expression constructs, expression vectors, fused proteins, transfected or transduced cells, in a form appropriate for the intended application. Generally, this will entail preparing compositions that are essentially free of pyrogens, as well as other impurities that could be harmful to humans or animals. The multimeric ligand, such as, for example, AP1903 (INN rimiducid, may be delivered, for example at doses of about 0.1 to 10 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

One may generally desire to employ appropriate salts and buffers to render delivery vectors stable and allow for uptake by target cells. Buffers also may be employed when recombinant cells are introduced into a patient. Aqueous compositions comprise an effective amount of the vector to cells, dissolved or dispersed in a pharmaceutically acceptable carrier or aqueous medium. Such compositions also are referred to as inocula. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

The active compositions may include classic pharmaceutical preparations. Administration of these compositions will be via any common route so long as the target tissue is available via that route. This includes, for example, oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, discussed herein.

The pharmaceutical forms suitable for injectable use include sterile aqueous solutions or dispersions and sterile powders for the extemporaneous preparation of sterile injectable solutions or dispersions. In all cases the form is sterile and is fluid to the extent that easy syringability exists. It is stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In certain examples, isotonic agents, for example, sugars or sodium chloride may be included. Prolonged absorption of the injectable compositions can be brought about by the use in the compositions of agents delaying absorption, for example, aluminum monostearate and gelatin.

For oral administration, the compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including, for example: gels, pastes, powders and slurries. The active ingredient may be added in a therapeutically effective amount to a paste dentifrice that may include, for example, water, binders, abrasives, flavoring agents, foaming agents, and humectants.

The compositions may be formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

### EXAMPLES

The examples set forth below illustrate certain embodiments and do not limit the technology.

Mechanisms for selectively ablating the donor cells have been studied as safety switches for cellular therapies, but there have been complications. Some experience with safety-switch genes to date has been in T lymphocytes since immunotherapy with these cells has proved efficacious as treatment for viral infections and malignancies (Walter, E. A., et al., N. Engl. J. Med. 1995, 333:1038-44; Rooney, C. M., et al., Blood. 1998, 92:1549-55; Dudley, M. E., et al., Science 2002, 298:850-54; Marjit, W. A., et al., Proc. Natl. Acad. Sci. USA 2003, 100:2742-47). The herpes simplex virus I-derived thymidine kinase (HSVTK) gene has been used as an in vivo suicide switch in donor T-cell infusions to treat recurrent malignancy and Epstein Barr virus (EBV) lymphoproliferation after hematopoietic stem cell transplantation (Bonini C, et al., Science. 1997, 276:1719-1724; Tiberghien P, et al., Blood. 2001, 97:63-72). However, destruction of T cells causing graft-versus-host disease was incomplete, and the use of gancyclovir (or analogs) as a pro-drug to activate HSV-TK precludes administration of gancyclovir as an antiviral drug for cytomegalovirus infections. This mechanism of action also requires interference with DNA synthesis, relying on cell division, so that cell killing may be protracted over several days and incomplete, producing a lengthy delay in clinical benefit (Ciceri, F., et al., Lancet Oncol. 2009, 262:1019-24). Moreover, HSV-TK-directed immune responses have resulted in elimination of HSV-TK-transduced cells, even in immunosuppressed human immunodeficiency virus and bone marrow transplant patients, compromising the persistence and hence efficacy of the infused T cells. HSV-TK is also virus-derived, and therefore potentially immunogenic (Bonini C, et al., Science. 1997, 276:1719-1724; Riddell S R, et al., Nat Med. 1996, 2:216-23). The E coli-derived cytosine deaminase gene has also been used clinically (Freytag S O, et al., Cancer Res. 2002, 62:4968-4976), but as a xenoantigen it may be immunogenic and thus incompatible with T-cell-based therapies that require long-term persistence. Transgenic human CD20, which can be activated by a monoclonal chimeric anti-CD20 antibody, has been proposed as a nonimmunogenic safety system (Introna M, et al., Hum Gene Ther. 2000, 11: 611-620).

The following section provides examples of method of providing a safety switch in cells used for cellular therapy, using a Caspase-9 chimeric protein.

### Example 1: Construction and Evaluation of Caspase-9 Suicide Switch Expression Vectors

Vector Construction and Confirmation of Expression

A safety switch that can be stably and efficiently expressed in human T cells is presented herein. The system includes human gene products with low potential immunogenicity that have been modified to interact with a small molecule dimerizer drug that is capable of causing the selective elimination of transduced T cells expressing the modified gene. Additionally, the inducible Caspase-9 maintains function in T cells overexpressing antiapoptotic molecules.

Expression vectors suitable for use as a therapeutic agent were constructed that included a modified human Caspase-9 activity fused to a human FK506 binding protein (FKBP), such as, for example, FKBP12v36. The Caspase-9/FK506 hybrid activity can be dimerized using a small molecule pharmaceutical. Full length, truncated, and modified versions of the Caspase-9 activity were fused to the ligand binding domain, or multimerizing region, and inserted into the retroviral vector MSCV.IRES.GRP, which also allows expression of the fluorescent marker, GFP. FIG. 1A illustrates the full length, truncated and modified Caspase-9 expression vectors constructed and evaluated as a suicide switch for induction of apoptosis.

The full-length inducible Caspase-9 molecule (F′-F-C-Casp9) includes 2, 3, or more FK506 binding proteins (FKBPs—for example, FKBP12v36 variants) linked with a Gly-Ser-Gly-Gly-Gly-Ser linker (SEQ ID NO: 285) to the small and large subunit of the Caspase molecule (see FIG. 1A). Full-length inducible Caspase-9 (F′F-C-Casp9.I.GFP) has a full-length Caspase-9, also includes a Caspase recruitment domain (CARD; GenBank NM001 229) linked to 2 12-kDa human FK506 binding proteins (FKBP12; GenBank AH002 818) that contain an F36V mutation (FIG. 1A). The amino acid sequence of one or more of the FKBPs (F′) was codon-wobbled (e.g., the 3¹d nucleotide of each amino acid codon was altered by a silent mutation that maintained the originally encoded amino acid) to prevent homologous recombination when expressed in a retrovirus. F′F-C-Casp9C3S includes a cysteine to serine mutation at position 287 that disrupts its activation site. In constructs F′F-Casp9, F-C-Casp9, and F′-Casp9, either the Caspase activation domain (CARD), one FKBP, or both, were deleted, respectively. All constructs were cloned into MSCV.IRES.GFP as EcoRI-XhoI fragments.

293T cells were transfected with each of these constructs and 48 hours after transduction expression of the marker gene GFP was analyzed by flow cytometry. In addition, 24 hours after transfection, 293T cells were incubated overnight with 100 nM CID and subsequently stained with the apoptosis marker annexin V. The mean and standard deviation of transgene expression level (mean GFP) and number of apoptotic cells before and after exposure to the chemical inducer of dimerization (CID) (% annexin V within GFP˜ cells) from 4 separate experiments are shown in the second through fifth columns of the table in FIG. 1A. In addition to the level of GFP expression and staining for annexin V, the expressed gene products of the full length, truncated and modified Caspase-9 were also analyzed by western blot to confirm the Caspase-9 genes were being expressed and the expressed product was the expected size. The results of the western blot are presented in FIG. 1B.

Coexpression of the inducible Caspase-9 constructs of the expected size with the marker gene GFP in transfected 293T cells was demonstrated by Western blot using a Caspase-9 antibody specific for amino acid residues 299-318, present both in the full-length and truncated Caspase molecules as well as a GFP-specific antibody. Western blots were performed as presented herein.

Transfected 293T cells were resuspended in lysis buffer (50% Tris/Gly, 10% sodium dodecyl sulfate [SDS], 4% beta-mercaptoethanol, 10% glycerol, 12% water, 4% bromophenol blue at 0.5%) containing aprotinin, leupeptin, and phenylmethylsulfonyl fluoride (Boehringer, Ingelheim, Germany) and incubated for 30 minutes on ice. After a 30-minute centrifugation, supernatant was harvested; mixed 1:2 with Laemmli buffer (Bio-Rad, Hercules, Calif.), boiled and loaded on a 10% SDS-polyacrylamide gel. The membrane was probed with rabbit anti-Caspase-9 (amino acid residues 299-3 18) immunoglobulin G (IgG; Affinity BioReagents, Golden, Colo.; 1:500 dilution) and with mouse anti-GFP IgG (Covance, Berkeley, Calif.; 1:25,000 dilution). Blots were then exposed to appropriate peroxidase-coupled secondary antibodies and protein expression was detected with enhanced chemiluminescence (ECL; Amersham, Arlington Heights, Ill.). The membrane was then stripped and reprobed with goat polyclonal antiactin (Santa Cruz Biotechnology; 1:500 dilution) to check equality of loading.

Additional smaller size bands, seem in FIG. 1B, likely represent degradation products. Degradation products for the F′F-C-Casp9 and F′F-Casp9 constructs may not be detected due to a lower expression level of these constructs as a result of their basal activity. Equal loading of each sample was confirmed by the substantially equal amounts of actin shown at the bottom of each lane of the western blot, indicating substantially similar amounts of protein were loaded in each lane.

An example of a chimeric polypeptide that may be expressed in the modified cells is provided herein. In this example, a single polypeptide is encoded by the nucleic acid vector. The inducible Caspase-9 polypeptide is separated from the CAR polypeptide during translation, due to skipping of a peptide bond. (Donnelly, M L 2001, J. Gen. Virol. 82:1013-25).

Evaluation of Caspase-9 Suicide Switch Expression Constructs.

### Cell Lines

B 95-8 EBV transformed B-cell lines (LCLs), Jurkat, and MT-2 cells (kindly provided by Dr S. Marriott, Baylor College of Medicine, Houston, Tex.) were cultured in RPMI 1640 (Hyclone, Logan, Utah) containing 10% fetal bovine serum (FBS; Hyclone). Polyclonal EBV-specific T-cell lines were cultured in 45% RPMI/45% Clicks (Irvine Scientific, Santa Ana, Calif.)/10% FBS and generated as previously reported. Briefly, peripheral blood mononuclear cells (2×10⁶ per well of a 24-well plate) were stimulated with autologous LCLs irradiated at 4000 rads at a responder-to-stimulator (R/S) ratio of 40:1. After 9 to 12 days, viable cells were restimulated with irradiated LCLs at an R/S ratio of 4:1. Subsequently, cytotoxic T cells (CTLs) were expanded by weekly restimulation with LCLs in the presence of 40 U/mL to 100 U/mL recombinant human interleukin-2 (rhIL-2; Proleukin; Chiron, Emeryville, Calif.).

### Retrovirus Transduction

For the transient production of retrovirus, 293T cells were transfected with iCasp9/iFas constructs, along with plasmids encoding gag-pol and RD 114 envelope using GeneJuice transfection reagent (Novagen, Madison, Wis.). Virus was harvested 48 to 72 hours after transfection, snap frozen, and stored at ˜80° C. until use. A stable FLYRD 18-derived retroviral producer line was generated by multiple transductions with VSV-G pseudotyped transient retroviral supernatant. FLYRD18 cells with highest transgene expression were single-cell sorted, and the clone that produced the highest virus titer was expanded and used to produce virus for lymphocyte transduction. The transgene expression, function, and retroviral titer of this clone was maintained during continuous culture for more than 8 weeks. For transduction of human lymphocytes, a non-tissue-culture-treated 24-well plate (Becton Dickinson, San Jose, Calif.) was coated with recombinant fibronectin fragment (FN CH-296; Retronectin; Takara Shuzo, Otsu, Japan; 4 □g/mL in PBS, overnight at 4° C.) and incubated twice with 0.5 mL retrovirus per well for 30 minutes at 37° C. Subsequently, 3×10⁵ to 5×10⁵ T cells per well were transduced for 48 to 72 hours using 1 mL virus per well in the presence of 100 U/mL IL-2. Transduction efficiency was determined by analysis of expression of the coexpressed marker gene green fluorescent protein (GFP) on a FACScan flow cytometer (Becton Dickinson). For functional studies, transduced CTLs were either non-selected or segregated into populations with low, intermediate, or high GFP expression using a MoFlo cytometer (Dako Cytomation, Ft Collins, Colo.) as indicated.

### Induction and Analysis of Apoptosis

CID (AP20187; ARIAD Pharmaceuticals) at indicated concentrations was added to transfected 293T cells or transduced CTLs. Adherent and nonadherent cells were harvested and washed with annexin binding buffer (BD Pharmingen, San Jose, Calif.). Cells were stained with annexin-V and 7-amino-actinomycin D (7-AAD) for 15 minutes according to the manufacturer's instructions (BD Pharmingen). Within 1 hour after staining, cells were analyzed by flow cytometry using CellQuest software (Becton Dickinson).

### Cytotoxicity Assay

The cytotoxic activity of each CTL line was evaluated in a standard 4-hour ⁵¹Cr release assay, as previously presented. Target cells included autologous LCLs, human leukocyte antigen (HLA) class I-mismatched LCLs and the lymphokine-activated killer cell-sensitive T-cell lymphoma line HSB-2. Target cells incubated in complete medium or 1% Triton X-100 (Sigma, St Louis, Mo.) were used to determine spontaneous and maximum ⁵¹Cr release, respectively. The mean percentage of specific lysis of triplicate wells was calculated as 100×(experimental release−spontaneous release)/(maximal release−spontaneous release).

### Phenotyping

Cell-surface phenotype was investigated using the following monoclonal antibodies: CD3, CD4, CD8 (Becton Dickinson) and CD56 and TCR-□/□ (Immunotech, Miami, Fla.). □NGFR-iFas was detected using anti-NGFR antibody (Chromaprobe, Aptos, Calif.). Appropriate matched isotype controls (Becton Dickinson) were used in each experiment. Cells were analyzed with a FACSscan flow cytometer (Becton Dickinson).

### Analysis of Cytokine Production

The concentration of interferon-□ (IFN-□□), IL-2, IL-4, IL-5, IL-10, and tumor necrosis factor-□□ (TNF□) in CTL culture supernatants was measured using the Human Th1/Th2 cytokine cytometric Bead Array (BD Pharmingen) and the concentration of IL-12 in the culture supernatants was measured by enzyme-linked immunosorbent assay (ELISA; R&D Systems, Minneapolis, Minn.) according to the instructions of the manufacturer.

### In Vivo Experiments

Non-obese diabetic severe combined immunodeficient (NOD/SCID) mice, 6 to 8 weeks of age, were irradiated (250 rad) and injected subcutaneously in the right flank with 10×10⁶ to 15×10⁶ LCLs resuspended in Matrigel (BD Bioscience). Two weeks later mice bearing tumors that were approximately 0.5 cm in diameter were injected into the tail vein with a 1:1 mixture of nontransduced and iCasp9.I.GFPhigh-transduced EBV CTLs (total 15×10⁶). At 4 to 6 hours prior and 3 days after CTL infusion, mice were injected intraperitoneally with recombinant hIL-2 (2000 U; Proleukin; Chiron). On day 4, the mice were randomly segregated in 2 groups: 1 group received CID (50 □g AP20187, intraperitoneally) and 1 group received carrier only (16.7% propanediol, 22.5% PEG400, and 1.25% Tween 80, intraperitoneally). On day 7, all mice were killed. Tumors were homogenized and stained with antihuman CD3 (BD Pharmingen). By FACS analysis, the number of GFP⁺ cells within the gated CD3⁺ population was evaluated. Tumors from a control group of mice that received only nontransduced CTLs (total 15×10⁶) were used as a negative control in the analysis of CD3⁺/GFP⁺ cells.

### Optimization of Expression and Function of Inducible Caspase-9

Caspases 3, 7, and 9 were screened for their suitability as inducible safety-switch molecules both in transfected 293T cells and in transduced human T cells. Only inducible Caspase-9 (iCasp9) was expressed at levels sufficient to confer sensitivity to the chosen CID (e.g., chemical inducer of dimerization). An initial screen indicated that the full length iCasp9 could not be maintained stably at high levels in T cells, possibly due to transduced cells being eliminated by the basal activity of the transgene. The CARD domain is involved in physiologic dimerization of Caspase-9 molecules, by a cytochrome C and adenosine triphosphate (ATP)-driven interaction with apoptotic protease-activating factor 1 (Apaf-1). Because of the use of a CID to induce dimerization and activation of the suicide switch, the function of the CARD domain is superfluous in this context and removal of the CARD domain was investigated as a method of reducing basal activity. Given that only dimerization rather than multimerization is required for activation of Caspase-9, a single FKBP12v36 domain also was investigated as a method to effect activation.

The activity of the resultant truncated and/or modified forms of Caspase-9 (e.g., the CARD domain, or one of the 2 FKBP domains, or both, are removed) were compared. A construct with a disrupted activation site, F′F-C-Casp9_{C->S}, provided a nonfunctional control (see FIG. 1A). All constructs were cloned into the retroviral vector MSCV²⁶ in which retroviral long terminal repeats (LTRs) direct transgene expression and enhanced GFP is coexpressed from the same mRNA by use of an internal ribosomal entry site (I RES). In transfected 293T cells, expression of all inducible Caspase-9 constructs at the expected size as well as coexpression of GFP was demonstrated by Western blot (see FIG. 1B). Protein expression (estimated by mean fluorescence of GFP and visualized on Western blot) was highest in the nonfunctional construct F′F-C-Casp9_{C->S} and greatly diminished in the full-length construct F′F-C-Casp9. Removal of the CARD (F′F-Casp9), one FKBP (F-C-Casp9), or both (F-Casp9) resulted in progressively higher expression of both inducible Caspase-9 and GFP, and correspondingly enhanced sensitivity to CID (see FIG. 1A). Based on these results, the F-Casp9 construct (henceforth referred to as iCasp9_{M}) was used for further study in human T lymphocytes.

Stable Expression of iCasp9_{M} in Human T Lymphocytes

The long-term stability of suicide gene expression is of utmost importance, since suicide genes must be expressed for as long as the genetically engineered cells persist. For T-cell transduction, a FLYRD18-derived retroviral producer clone that produces high-titer RD114-pseudotyped virus was generated to facilitate the transduction of T cells. iCasp9_{M} expression in EBV-specific CTL lines (EBV-CTL) was evaluated since EBV-specific CTL lines have well-characterized function and specificity and are already being used as in vivo therapy for prevention and treatment of EBV-associated malignancies. Consistent transduction efficiencies of EBV-CTLs of more than 70% (mean, 75.3%; range, 71.4%-83.0% in 5 different donors) were obtained after a single transduction with retrovirus. The expression of iCasp9_{M} in EBV-CTLs was stable for at least 4 weeks after transduction without selection or loss of transgene function.

iCasp9_{M} does not Alter Transduced T-Cell Characteristics

To ensure that expression of iCasp9_{M} did not alter T-cell characteristics, the phenotype, antigen-specificity, proliferative potential, and function of nontransduced or nonfunctional iCasp9_{C->S}-transduced EBV-CTLs was compared with that of iCasp9_{M}-transduced EBV-CTLs. In 4 separate donors, transduced and nontransduced CTLs consisted of equal numbers of CD4⁺, CD8⁺, CD56⁺, and TCR□□/□+ cells. Similarly, production of cytokines including IFN-□, TNF□, IL-10, IL-4, IL-5, and IL-2 was unaltered by iCasp9_{M} expression. iCasp9_{M}-transduced EBV-CTLs specifically lysed autologous LCLs comparable to nontransduced and control-transduced CTLs. Expression of iCasp9M did not affect the growth characteristics of exponentially growing CTLs, and importantly, dependence on antigen and IL-2 for proliferation was preserved. On day 21 after transduction, the normal weekly antigenic stimulation with autologous LCLs and IL-2 was continued or discontinued. Discontinuation of antigen stimulation resulted in a steady decline of T cells.

Elimination of More than 99% of T Lymphocytes Selected for High Transgene Expression In Vitro

Inducible iCasp9_{M} proficiency in CTLs was tested by monitoring loss of GFP-expressing cells after administration of CID; 91.3% (range, 89.5%-92.6% in 5 different donors) of GFP⁺ cells were eliminated after a single 10-nM dose of CID. Similar results were obtained regardless of exposure time to CID (range, 1 hour-continuous). In all experiments, CTLs that survived CID treatment had low transgene expression with a 70% (range, 55%-82%) reduction in mean fluorescence intensity of GFP after CID. No further elimination of the surviving GFP⁺ T cells could be obtained by an antigenic stimulation followed by a second 10-nM dose of CID. Therefore, the non-responding CTLs most likely expressed insufficient iCasp9_{M} for functional activation by CID. To investigate the correlation between low levels of expression and CTL non-response to CID, CTLs were sorted for low, intermediate, and high expression of the linked marker gene GFP and mixed 1:1 with nontransduced CTLs from the same donor to allow for an accurate quantitation of the number of transduced T cells responding to CID-induced apoptosis.

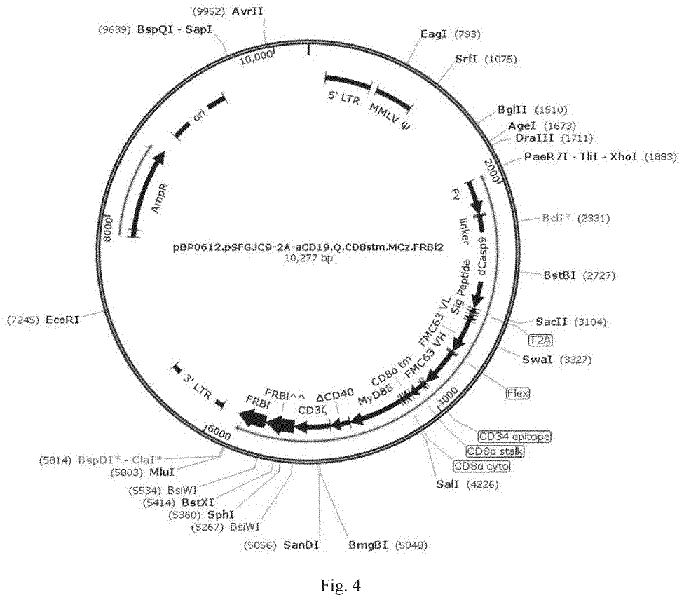

The number of transduced T cells eliminated increased with the level of GFP transgene expression (see FIGS. 4A, 4B and 4C). To determine the correlation between transgene expression and function of iCasp9_{M}, iCasp9_{M} IRES.GFP-transduced EBV-CTL were selected for low (mean 21), intermediate (mean 80) and high (mean 189) GFP expression. Selected T-cells were incubated overnight with 10 nM CID and subsequently stained with annexin V and 7-AAD. Indicated are the percentages of annexin V+/7-AAD− and annexin V+/7-AAD+ T−. Selected T-cells were mixed 1:1 with non-transduced T-cells and incubated with 10 nM CID following antigenic stimulation. Indicated is the percentage of residual GFP-positive T-cells on day 7.

For GFP_{high}-selected cells, 10 nM CID led to deletion of 99.1% (range, 98.7%-99.4%) of transduced cells. On the day of antigen stimulation, F-Casp9_{M}.I.GFP-transduced CTLs were either untreated or treated with 10 nM CID. Seven days later, the response to CID was measured by flow cytometry for GFP. The percentage of transduced T cells was adjusted to 50% to allow for an accurate measurement of residual GFP⁺ cells after CID treatment. The responses to CID in unselected (top row of and GFP_{high}-selected CTLs (bottom row of was compared. The percentage of residual GFP⁺ cells is indicated.

Rapid induction of apoptosis in the GFP_{high}-selected cells is demonstrated by apoptotic characteristics such as cell shrinkage and fragmentation within 14 hours of CID administration. After overnight incubation with 10 nM CID, F-Casp9_{M}.I.GFP_{high}-transduced T cells had apoptotic characteristics such as cell shrinkage and fragmentation by microscopic evaluation. Of the T cells selected for high expression, 64% (range, 59%-69%) had an apoptotic (annexin-V++/7-AAD⁻) and 30% (range, 26%-32%) had a necrotic (annexinV+/7-AAD+) phenotype. Staining with markers of apoptosis showed that 64% of T cells had an apoptotic phenotype (annexin V+, 7-AAD⁻, lower right quadrant) and 32% a necrotic phenotype (annexin V+, 7-AAD+, upper right quadrant). A representative example of 3 separate experiments is shown.

In contrast, the induction of apoptosis was significantly lower in T cells selected for intermediate or low GFP expression (see FIGS. 4A, 4B and 4C). For clinical applications therefore, versions of the expression constructs with selectable markers that allow selection for high copy number, high levels of expression, or both high copy number and high levels of expression may be desirable. CID-induced apoptosis was inhibited by the panCaspase inhibitor zVAD-fmk (100 μM for 1 hour prior to adding CID. Titration of CID showed that 1 nM CID was sufficient to obtain the maximal deletion effect. A dose-response curve using the indicated amounts of CID (AP20187) shows the sensitivity of F-Casp9_{M}.I.GFP_{high} to CID. Survival of GFP⁺ cells is measured on day 7 after administration of the indicated amount of CID. The mean and standard deviation for each point are given. Similar results were obtained using another chemical inducer of dimerization (CID), AP1903, which was clinically shown to have substantially no adverse effects when administered to healthy volunteers. The dose response remained unchanged for at least 4 weeks after transduction.

iCasp9_{M} is Functional in Malignant Cells that Express Antiapoptotic Molecules

Caspase-9 was selected as an inducible proapoptotic molecule for clinical use rather than previously presented iFas and iFADD, because Caspase-9 acts relatively late in apoptosis signaling and therefore is expected to be less susceptible to inhibition by apoptosis inhibitors. Thus, suicide function should be preserved not only in malignant, transformed T-cell lines that express antiapoptotic molecules, but also in subpopulations of normal T cells that express elevated antiapoptotic molecules as part of the process to ensure long-term preservation of memory cells. To further investigate the hypothesis, the function of iCasp9_{M} and iFas was first compared in EBV-CTLs. To eliminate any potential vector based difference, inducible Fas also was expressed in the MSCV.IRES.GFP vector, like iCasp9. For these experiments both ΔNGFR.iFas.I.GFP and iCasp9_{M}.I.GFP-transduced CTLs were sorted for GFP_{high} expression and mixed with nontransduced CTLs at a 1:1 ratio to obtain cell populations that expressed either iFas or iCasp9_{M} at equal proportions and at similar levels. The EBV-CTLs were sorted for high GFP expression and mixed 1:1 with nontransduced CTLs as presented. The percentages of ΔNGFR⁺/GFP⁺ and GFP⁺ T cells are indicated.

Elimination of GFP⁺ cells after administration of 10 nM CID was more rapid and more efficient in iCasp9_{M} than in iFas-transduced CTLs (99.2%+/−0.14% of iCasp9_{M}-transduced cells compared with 89.3%+/−4.9% of iFas-transduced cells at day 7 after CID; P<0.05). On the day of LCL stimulation, 10 nM CID was administered, and GFP was measured at the time points indicated to determine the response to CID. Black diamonds represent data for ΔNGFR-iFas.I.GFP; black squares represent data for iCasp9_{M}.I.GFP. Mean and standard deviation of 3 experiments are shown.

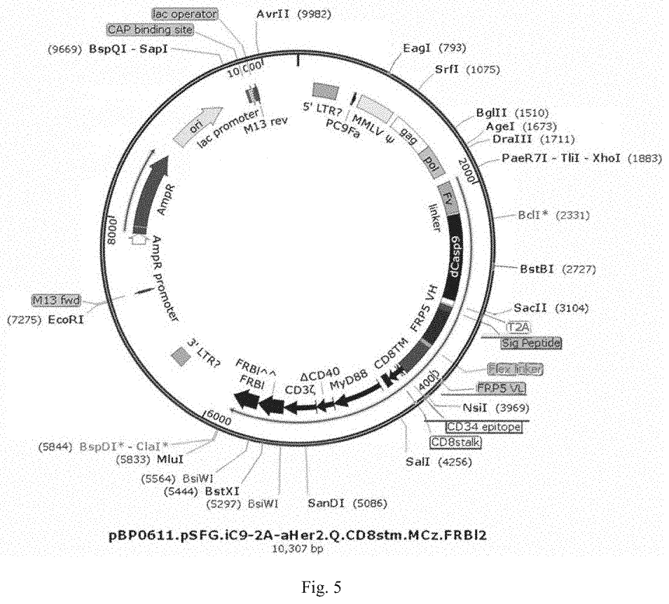

The function of iCasp9M and iFas was also compared in 2 malignant T-cell lines: Jurkat, an apoptosis-sensitive T-cell leukemia line, and MT-2, an apoptosis-resistant T-cell line, due to c-FLIP and bcl-xL expression. Jurkat cells and MT-2 cells were transduced with iFas and iCasp9_{M} with similar efficiencies (92% vs 84% in Jurkat, 76% vs 70% in MT-2) and were cultured in the presence of 10 nM CID for 8 hours. Annexin-V staining showed that although iFas and iCasp9_{M} induced apoptosis in an equivalent number of Jurkat cells (56.4%+/−15.6% and 57.2%+1-18.9%, respectively), only activation of iCasp9_{M} resulted in apoptosis of MT-2 cells (19.3%+/−8.4% and 57.9%+/−11.9% for iFas and iCasp9_{M}, respectively; see FIG. 5C).

The human T-cell lines Jurkat (left) and MT-2 (right) were transduced with ΔNGFR-iFas.I.GFP or iCasp9_{M}.I.GFP. An equal percentage of T cells were transduced with each of the suicide genes: 92% for ΔNGFR-iFas.I.GFP versus 84% for iCasp9_{M}.I.GFP in Jurkat, and 76% for ΔNGFR-iFas.I.GFP versus 70% for iCasp9_{M}.I.GFP in MT-2. T cells were either nontreated or incubated with 10 nM CID. Eight hours after exposure to CID, apoptosis was measured by staining for annexin V and 7-AAD. A representative example of 3 experiments is shown. PE indicates phycoerythrin. These results demonstrate that in T cells overexpressing apoptosis-inhibiting molecules, the function of iFas can be blocked, while iCasp9_{M} can still effectively induce apoptosis.

iCasp9M-Mediated Elimination of T Cells Expressing an Immunomodulatory Transgene

To determine whether iCasp9M could effectively destroy cells genetically modified to express an active transgene product, the ability of iCasp9_{M} to eliminate EBV-CTLs stably expressing IL-12 was measured. While IL-12 was undetectable in the supernatant of nontransduced and iCasp9_{M}.IRES.GFP-transduced CTLs, the supernatant of iCasp9_{M}.IRES.IL-12-transduced cells contained 324 pg/mL to 762 pg/mL IL-12. After administration of 10 nM CID, however, the IL-12 in the supernatant fell to undetectable levels (<7.8 pg/mL). Thus, even without prior sorting for high transgene expressing cells, activation of iCasp9_{M} is sufficient to completely eliminate all T cells producing biologically relevant levels of IL-12. The marker gene GFP in the iCasp9_{M}.I.GFP constructs was replaced by flexi IL-12, encoding the p40 and p35 subunits of human IL-12. iCasp9_{M}.I.GFP- and iCasp9_{M}.I.IL-12-transduced EBV-CTLs were stimulated with LCLs, and then left untreated or exposed to 10 nM CID. Three days after a second antigenic stimulation, the levels of IL-12 in the culture supernatant were measured by IL-12 ELISA (detection limit of this assay is 7.8 pg/mL). The mean and standard deviation of triplicate wells are indicated. Results of 1 of 2 experiments with CTLs from 2 different donors are shown.

Elimination of More than 99% of T Cells Selected for High Transgene Expression In Vivo

The function of iCasp9_{M} also was evaluated in transduced EBV-CTLs in vivo. A SCID mouse-human xenograft model was used for adoptive immunotherapy. After intravenous infusion of a 1:1 mixture of nontransduced and iCasp9_{M}.IRES.GFP_{high}-transduced CTLs into SCID mice bearing an autologous LCL xenograft, mice were treated either with a single dose of CID or carrier only. Three days after CID/carrier administration, tumors were analyzed for human CD3⁺/GFP⁺ cells. Detection of the nontransduced component of the infusion product, using human anti-CD3 antibodies, confirmed the success of the tail-vein infusion in mice that received CID. In mice treated with CID, there was more than a 99% reduction in the number of human CD3⁺/GFP⁺ T cells, compared with infused mice treated with carrier alone, demonstrating equally high sensitivity of iCasp9_{M}-transduced T cells in vivo and in vitro.

The function of iCasp9_{M} in vivo, was assayed. NOD/SCID mice were irradiated and injected subcutaneously with 10×10⁶ to 15×10⁶ LCLs. After 14 days, mice bearing tumors of 0.5 cm in diameter received a total of 15×10⁶ EBV-CTLs (50% of these cells were nontransduced and 50% were transduced with iCasp9_{M}.I.GFP and sorted for high GFP expression). On day 3 after CTL administration, mice received either CID (50 □g AP20187; (black diamonds, n=6) or carrier only (black squares, n=5) and on day 6 the presence of human CD3⁺/GFP⁺ T cells in the tumors was analyzed. Human CD3⁺ T cells isolated from the tumors of a control group of mice that received only nontransduced CTLs (15×10⁶ CTLs; n=4) were used as a negative control for the analysis of CD3⁺/GFP⁺ T cells within the tumors.

### Discussion

Presented herein are expression vectors expressing suicide genes suitable for eliminating gene-modified T cells in vivo, in some embodiments. Suicide gene expression vectors presented herein have certain non-limiting advantageous features including stable coexpression in all cells carrying the modifying gene, expression at levels high enough to elicit cell death, low basal activity, high specific activity, and minimal susceptibility to endogenous antiapoptotic molecules. Presented herein, in certain embodiments, is an inducible Caspase-9, iCasp9_{M}, which has low basal activity allowing stable expression for more than 4 weeks in human T cells. A single 10-nM dose of a small molecule chemical inducer of dimerization (CID) is sufficient to kill more than 99% of iCasp9_{M}-transduced cells selected for high transgene expression both in vitro and in vivo. Moreover, when coexpressed with Th1 cytokine IL-12, activation of iCasp9_{M} eliminated all detectable IL-12-producing cells, even without selection for high transgene expression. Caspase-9 acts downstream of most antiapoptotic molecules, therefore, a high sensitivity to CID is preserved regardless of the presence of increased levels of antiapoptotic molecules of the bcl-2 family. Thus, iCasp9_{M} also may prove useful for inducing destruction even of transformed T cells and memory T cells that are relatively resistant to apoptosis.

Unlike other Caspase molecules, proteolysis does not appear sufficient for activation of Caspase-9. Crystallographic and functional data indicate that dimerization of inactive Caspase-9 monomers leads to conformational change-induced activation. The concentration of pro-Caspase-9, in a physiologic setting, is in the range of about 20 nM, well below the threshold needed for dimerization.

Without being limited by theory, it is believed the energetic barrier to dimerization can be overcome by homophilic interactions between the CARD domains of Apaf-1 and Caspase-9, driven by cytochrome C and ATP. Overexpression of Caspase-9 joined to 2 FKBPs may allow spontaneous dimerization to occur and can account for the observed toxicity of the initial full length Caspase-9 construct. A decrease in toxicity and an increase in gene expression was observed following removal of one FKBP, most likely due to a reduction in toxicity associated with spontaneous dimerization. While multimerization often is involved in activation of surface death receptors, dimerization of Caspase-9 should be sufficient to mediate activation. Data presented herein indicates that iCasp9 constructs with a single FKBP function as effectively as those with 2 FKBPs. Increased sensitivity to CID by removal of the CARD domain may represent a reduction in the energetic threshold of dimerization upon CID binding.

The persistence and function of virus- or bacteria-derived lethal genes, such as HSV-TK and cytosine deaminase, can be impaired by unwanted immune responses against cells expressing the virus or bacteria derived lethal genes. The FKBPs and proapoptotic molecules that form the components of iCasp9_{M} are human-derived molecules and are therefore less likely to induce an immune response. Although the linker between FKBP and Caspase-9 and the single point mutation in the FKBP domain introduce novel amino acid sequences, the sequences were not immunologically recognized by macaque recipients of iFas-transduced T cells. Additionally, because the components of iCasp9_{M} are human-derived molecules, no memory T cells specific for the junction sequences should be present in a recipient, unlike virus-derived proteins such as HSV-TK, thereby reducing the risk of immune response-mediated elimination of iCasp9_{M}-transduced T cells.

Previous studies using inducible Fas or the death effector domains (DED) of Fas associated death domain proteins (FADD) showed that approximately 10% of transduced cells were unresponsive to activation of the destructive gene. As observed in experiments presented here, a possible explanation for unresponsiveness to CID is low expression of the transgene. The iCasp9_{M}-transduced T cells in our study and iFas-transduced T cells in studies by others that survived after CID administration had low levels of transgene expression. In an attempt to overcome a perceived retroviral “positional effect”, increased levels of homogeneous expression of the transgene were achieved by flanking retroviral integrants with the chicken beta-globin chromatin insulator. Addition of the chromatin insulator dramatically increased the homogeneity of expression in transduced 293T cells, but had no significant effect in transduced primary T cell. Selection of T cells with high expression levels minimized variability of response to the dimerizer. Over 99% of transduced T cells sorted for high GFP expression were eliminated after a single 10-nM CID dose. This demonstration supports the hypothesis that cells expressing high levels of suicide gene can be isolated using a selectable marker.

A very small number of resistant residual cells may cause a resurgence of toxicity, a deletion efficiency of up to 2 logs will significantly decrease this possibility. For clinical use, coexpression with a nonimmunogenic selectable marker such as truncated human NGFR, CD20, or CD34 (e.g., instead of GFP) will allow for selection of high transgene-expressing T cells. Coexpression of the suicide switch (e.g., iCASP9_{M}) and a suitable selectable marker (e.g., truncated human NGFR, CD20, CD34, the like and combinations thereof) can be obtained using either an internal ribosome entry site (IRES) or posttranslational modification of a fusion protein containing a self-cleaving sequence (eg, 2A). In contrast, in situations where the sole safety concern is the transgene-mediated toxicity (eg, artificial T-cell receptors, cytokines, the like or combinations thereof), this selection step may be unnecessary, as tight linkage between iCasp9_{M} and transgene expression enables elimination of substantially all cells expressing biologically relevant levels of the therapeutic transgene. This was demonstrated by coexpressing iCasp9_{M} with IL-12. Activation of iCasp9_{M} substantially eliminated any measurable IL-12 production. The success of transgene expression and subsequent activation of the “suicide switch” may depend on the function and the activity of the transgene.

Another possible explanation for unresponsiveness to CID is that high levels of apoptosis inhibitors may attenuate CID-mediated apoptosis. Examples of apoptosis inhibitors include c-FLIP, bcl-2 family members and inhibitors of apoptosis proteins (IAPs), which normally regulate the balance between apoptosis and survival. For instance, upregulation of c-FLIP and bcl-2 render a subpopulation of T cells, destined to establish the memory pool, resistant to activation-induced cell death in response to cognate target or antigen-presenting cells. In several T-lymphoid tumors, the physiologic balance between apoptosis and survival is disrupted in favor of cell survival. A suicide gene should delete substantially all transduced T cells including memory and malignantly transformed cells. Therefore, the chosen inducible suicide gene should retain a significant portion if not substantially all of its activity in the presence of increased levels of antiapoptotic molecules.

The apical location of iFas (or iFADD) in the apoptosis signaling pathway may leave it especially vulnerable to inhibitors of apoptosis, thus making these molecules less well suited to being the key component of an apoptotic safety switch. Caspase 3 or 7 would seem well suited as terminal effector molecules; however neither could be expressed at functional levels in primary human T cells. Therefore Caspase-9, was chosen as the suicide gene, because Caspase-9 functions late enough in the apoptosis pathway that it bypasses the inhibitory effects of c-FLIP and antiapoptotic bcl-2 family members, and Caspase-9 also could be expressed stably at functional levels. Although X-linked inhibitor of apoptosis (XIAP) could in theory reduce spontaneous Caspase-9 activation, the high affinity of AP20187 (or AP1903) for FKBPv36 may displace this noncovalently associated XIAP. In contrast to iFas, iCasp9_{M} remained functional in a transformed T-cell line that overexpresses antiapoptotic molecules, including bcl-xL.

Presented herein is an inducible safety switch, designed specifically for expression from an oncoretroviral vector by human T cells. iCasp9_{M} can be activated by AP1903 (or analogs), a small chemical inducer of dimerization that has proven safe at the required dose for optimum deletional effect, and unlike ganciclovir or rituximab has no other biologic effects in vivo. Therefore, expression of this suicide gene in T cells for adoptive transfer can increase safety and also may broaden the scope of clinical applications.

### Example 2: Using the iCasp9 Suicide Gene to Improve the Safety of Allodepleted T Cells after Haploidentical Stem Cell Transplantation

Presented in this example are expression constructs and methods of using the expression constructs to improve the safety of allodepleted T cells after haploidentical stem cell transplantation. A retroviral vector encoding iCasp9 and a selectable marker (truncated CD19) was generated as a safety switch for donor T cells. Even after allodepletion (using anti-CD25 immunotoxin), donor T cells could be efficiently transduced, expanded, and subsequently enriched by CD19 immunomagnetic selection to >90% purity. The engineered cells retained anti-viral specificity and functionality, and contained a subset with regulatory phenotype and function. Activating iCasp9 with a small-molecule dimerizer rapidly produced >90% apoptosis. Although transgene expression was downregulated in quiescent T cells, iCasp9 remained an efficient suicide gene, as expression was rapidly upregulated in activated (alloreactive) T cells.

### Materials and Methods

### Generation of Allodepleted T Cells

Allodepleted cells were generated from healthy volunteers as previously presented. Briefly, peripheral blood mononuclear cells (PBMCs) from healthy donors were co-cultured with irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that expressed CD25 were depleted from the co-culture by overnight incubation in RFT5-SMPT-dgA immunotoxin. Allodepletion was considered adequate if the residual CD3⁺CD25⁺ population was <1% and residual proliferation by 3H-thymidine incorporation was <10%.

### Plasmid and Retrovirus

SFG.iCasp9.2A.CD19 consists of inducible Caspase-9 (iCasp9) linked, via a cleavable 2A-like sequence, to truncated human CD19. iCasp9 consists of a human FK5 06-binding protein (FKBP12; GenBank AH002 818) with an F36V mutation, connected via a Ser-Gly-Gly-Gly-Ser linker (SEQ ID NO: 286) to human Caspase-9 (CASP9; GenBank NM 001229). The F36V mutation increases the binding affinity of FKBP12 to the synthetic homodimerizer, AP20187 or AP1903. The Caspase recruitment domain (CARD) has been deleted from the human Caspase-9 sequence because its physiological function has been replaced by FKBP12, and its removal increases transgene expression and function. The 2A-like sequence encodes an 20 amino acid peptide from Thosea asigna insect virus, which mediates >99% cleavage between a glycine and terminal proline residue, resulting in 19 extra amino acids in the C terminus of iCasp9, and one extra proline residue in the N terminus of CD19. CD19 consists of full-length CD19 (GenBank NM 001770) truncated at amino acid 333 (TDPTRRF (SEQ ID NO: 290)), which shortens the intracytoplasmic domain from 242 to 19 amino acids, and removes all conserved tyrosine residues that are potential sites for phosphorylation.

A stable PG13 clone producing Gibbon ape leukemia virus (Gal-V) pseudotyped retrovirus was made by transiently transfecting Phoenix Eco cell line (ATCC product #5D3444; ATCC, Manassas, Va.) with SFG.iCasp9.2A.CD19. This produced Eco-pseudotyped retrovirus. The PG13 packaging cell line (ATCC) was transduced three times with Eco-pseudotyped retrovirus to generate a producer line that contained multiple SFG.iCasp9.2A.CD19 proviral integrants per cell. Single cell cloning was performed, and the PG13 clone that produced the highest titer was expanded and used for vector production.

### Retroviral Transduction

Culture medium for T cell activation and expansion consisted of 45% RPMI 1640 (Hyclone, Logan, Utah), 45% Clicks (Irvine Scientific, Santa Ana, Calif.) and 10% fetal bovine serum (FBS; Hyclone). Allodepleted cells were activated by immobilized anti-CD3 (OKT3; Ortho Biotech, Bridgewater, N.J.) for 48 hours before transduction with retroviral vector. Selective allodepletion was performed by co-culturing donor PBMC with recipient EBV-LCL to activate alloreactive cells: activated cells expressed CD25 and were subsequently eliminated by anti-CD25 immunotoxin. The allodepleted cells were activated by OKT3 and transduced with the retroviral vector 48 hours later. Immunomagnetic selection was performed on day 4 of transduction; the positive fraction was expanded for a further 4 days and cryopreserved.

In small-scale experiments, non-tissue culture-treated 24-well plates (Becton Dickinson, San Jose, Calif.) were coated with OKT3 1 g/ml for 2 to 4 hours at 37° C. Allodepleted cells were added at 1×10⁶ cells per well. At 24 hours, 100 U/ml of recombinant human interleukin-2 (IL-2) (Proleukin; Chiron, Emeryville, Calif.) was added. Retroviral transduction was performed 48 hours after activation. Non-tissue culture-treated 24-well plates were coated with 3.5 □g/cm² recombinant fibronectin fragment (CH-296; Retronectin; Takara Mirus Bio, Madison, Wis.) and the wells loaded twice with retroviral vector-containing supernatant at 0.5 ml per well for 30 minutes at 37° C., following which OKT3-activated cells were plated at 5×10⁵ cells per well in fresh retroviral vector-containing supernatant and T cell culture medium at a ratio of 3:1, supplemented with 100 U/ml IL-2. Cells were harvested after 2 to 3 days and expanded in the presence of 50 U/ml IL-2.

Scaling-Up Production of Gene-Modified Allodepleted Cells

Scale-up of the transduction process for clinical application used non-tissue culture-treated T75 flasks (Nunc, Rochester, N.Y.), which were coated with 10 ml of OKT3 1 □g/ml or 10 ml of fibronectin 7 □g/ml at 4° C. overnight. Fluorinated ethylene propylene bags corona-treated for increased cell adherence (2PF-0072AC, American Fluoroseal Corporation, Gaithersburg, Md.) were also used. Allodepleted cells were seeded in OKT3-coated flasks at 1×10⁶ cells/ml. 100 U/ml IL-2 was added the next day. For retroviral transduction, retronectin-coated flasks or bags were loaded once with 10 ml of retrovirus-containing supernatant for 2 to 3 hours. OKT3-activated T cells were seeded at 1×10⁶ cells/ml in fresh retroviral vector-containing medium and T cell culture medium at a ratio of 3:1, supplemented with 100 U/ml IL-2. Cells were harvested the following morning and expanded in tissue-culture treated T75 or T175 flasks in culture medium supplemented with between about 50 to 100 U/ml IL-2 at a seeding density of between about 5×10⁵ cells/ml to 8×10⁵ cells/ml.

### CD19 Immunomagnetic Selection

Immunomagnetic selection for CD19 was performed 4 days after transduction. Cells were labeled with paramagnetic microbeads conjugated to monoclonal mouse anti-human CD19 antibodies (Miltenyi Biotech, Auburn, Calif.) and selected on MS or LS columns in small scale experiments and on a CliniMacs Plus automated selection device in large scale experiments. CD19-selected cells were expanded for a further 4 days and cryopreserved on day 8 post transduction. These cells were referred to as “gene-modified allodepleted cells”.

### Immunophenotyping and Pentamer Analysis

Flow cytometric analysis (FACSCalibur and CellQuest software; Becton Dickinson) was performed using the following antibodies: CD3, CD4, CD8, CD19, CD25, CD27, CD28, CD45RA, CD45RO, CD56 and CD62L. CD19-PE (Clone 4G7; Becton Dickinson) was found to give optimum staining and was used in all subsequent analysis. A Non-transduced control was used to set the negative gate for CD19. An HLA-pentamer, HLA-B8-RAKFKQLL (SEQ ID NO: 287) (Proimmune, Springfield, Va.) was used to detect T cells recognizing an epitope from EBV lytic antigen (BZLF1). HLA-A2-NLVPMVATV (SEQ ID NO: 288) pentamer was used to detect T cells recognizing an epitope from CMV-pp65 antigen.

### Interferon-ELISpot Assay for Anti-Viral Response

Interferon-ELISpot for assessment of responses to EBV, CMV and adenovirus antigens was performed using known methods. Gene-modified allodepleted cells cryopreserved at 8 days post-transduction were thawed and rested overnight in complete medium without IL-2 prior to use as responder cells. Cryopreserved PBMCs from the same donor were used as comparators. Responder cells were plated in duplicate or triplicate in serial dilutions of 2×10⁵, 1×10⁵, 5×10⁴ and 2.5×10⁴ cells per well. Stimulator cells were plated at 1×10⁵ per well. For response to EBV, donor-derived EBV-LCLs irradiated at 40 Gy were used as stimulators. For response to adenovirus, donor-derived activated monocytes infected with Ad5f35 adenovirus were used.

Briefly, donor PBMCs were plated in X-Vivo 15 (Cambrex, Walkersville, Md.) in 24-well plates overnight, harvested the next morning, infected with Ad5f35 at a multiplicity of infection (MOI) of 200 for 2 hours, washed, irradiated at 30 Gy, and used as stimulators. For anti-CMV response, a similar process using Ad5f35 adenovirus encoding the CMV pp65 transgene (Ad5f35-pp65) at an MOI of 5000 was used. Specific spot-forming units (SFU) were calculated by subtracting SFU from responder-alone and stimulator-alone wells from test wells. Response to CMV was the difference in SFU between Ad5f35-pp65 and Ad5f35 wells.

### EBV-Specific Cytotoxicity

Gene-modified allodepleted cells were stimulated with 40 Gy-irradiated donor-derived EBVLCL at a responder: stimulator ratio of 40:1. After 9 days, the cultures were restimulated at a responder: stimulator ratio of 4:1. Restimulation was performed weekly as indicated. After two or three rounds of stimulation, cytotoxicity was measured in a 4-hour 51 Cr-release assay, using donor EBV-LCL as target cells and donor OKT3 blasts as autologous controls. NK activity was inhibited by adding 30-fold excess of cold K562 cells.

Induction of Apoptosis with Chemical Inducer of Dimerization, AP20187

Suicide gene functionality was assessed by adding a small molecule synthetic homodimerizer, AP20187 (Ariad Pharmaceuticals; Cambridge, Mass.), at 10 nM final concentration the day following CD19 immunomagnetic selection. Cells were stained with annexin V and 7-amino-actinomycin (7-AAD)(BD Pharmingen) at 24 hours and analyzed by flow cytometry. Cells negative for both annexin V and 7-AAD were considered viable, cells that were annexin V positive were apoptotic, and cells that were both annexin V and 7-AAD positive were necrotic. The percentage killing induced by dimerization was corrected for baseline viability as follows: Percentage killing=100%−(% Viability in AP20187-treated cells÷% Viability in nontreated cells).

### Assessment of Transgene Expression Following Extended Culture and Reactivation

Cells were maintained in T cell medium containing 50 U/ml IL-2 until 22 days after transduction. A portion of cells was reactivated on 24-well plates coated with 1 g/ml OKT3 and 1 □g/ml anti-CD28 (Clone CD28.2, BD Pharmingen, San Jose, Calif.) for 48 to 72 hours. CD19 expression and suicide gene function in both reactivated and non-reactivated cells were measured on day 24 or 25 post transduction.

In some experiments, cells also were cultured for 3 weeks post transduction and stimulated with 30G□ irradiated allogeneic PBMC at a responder: stimulator ratio of 1:1. After 4 days of co-culture, a portion of cells was treated with 10 nM AP20187. Killing was measured by annexin V/7-AAD staining at 24 hours, and the effect of dimerizer on bystander virus-specific T cells was assessed by pentamer analysis on AP20187-treated and untreated cells.

### Regulatory T Cells

CD4, CD25 and Foxp3 expression was analyzed in gene-modified allodepleted cells using flow cytometry. For human Foxp3 staining, the eBioscience (San Diego, Calif.) staining set was used with an appropriate rat IgG2a isotype control. These cells were co-stained with surface CD25-FITC and CD4-PE. Functional analysis was performed by co-culturing CD4⁺25⁺ cells selected after allodepletion and gene modification with carboxyfluorescein diacetate N-succinimidyl ester (CFSE)-labeled autologous PBMC. CD4⁺25⁺ selection was performed by first depleting CD8⁺ cells using anti-CD 8 microbeads (Miltenyi Biotec, Auburn, Calif.), followed by positive selection using anti-CD25 microbeads (Miltenyi Biotec, Auburn, Calif.). CFSE-labeling was performed by incubating autologous PBMC at 2×10⁷/ml in phosphate buffered saline containing 1.5 □M CFSE for 10 minutes. The reaction was stopped by adding an equivalent volume of FBS and incubating for 10 minutes at 37° C. Cells were washed twice before use. CFSE-labeled PBMCs were stimulated with OKT3 500 ng/ml and 40G-irradiated allogeneic PBMC feeders at a PBMC:allogeneic feeder ratio of 5:1. The cells were then cultured with or without an equal number of autologous CD4⁺25⁺ gene-modified allodepleted cells. After 5 days of culture, cell division was analyzed by flow cytometry; CD19 was used to gate out non-CFSE-labeled CD4⁺CD25⁺ gene-modified T cells.

### Statistical Analysis

Paired, 2-tailed Student's t test was used to determine the statistical significance of differences between samples. All data are represented as mean±1 standard deviation.

### Results

Selectively allodepleted T cells can be efficiently transduced with iCasp9 and expanded

Selective allodepletion was performed in accordance with clinical protocol procedures. Briefly, 3/6 to 5/6 HLA-mismatched PBMC and lymphoblastoid cell lines (LCL) were co-cultured. RFT5-SMPT-dgA immunotoxin was applied after 72 hours of co-culture and reliably produced allodepleted cells with <10% residual proliferation (mean 4.5±2.8%; range 0.74 to 9.1%; 10 experiments) and containing <1% residual CD3⁺CD25⁺ cells (mean 0.23±0.20%; range 0.06 to 0.73%; 10 experiments), thereby fulfilling the release criteria for selective allodepletion, and serving as starting materials for subsequent manipulation.

Allodepleted cells activated on immobilized OKT3 for 48 hours could be efficiently transduced with Gal-V pseudotyped retrovirus vector encoding SFG.iCasp9.2A.CD19. Transduction efficiency assessed by FACS analysis for CD19 expression 2 to 4 days after transduction was about 53%±8%, with comparable results for small-scale (24-well plates) and large-scale (T75 flasks) transduction (about 55±8% versus about 50%±10% in 6 and 4 experiments, respectively). Cell numbers contracted in the first 2 days following OKT3 activation such that only about 61%±12% (range of about 45% to 80%) of allodepleted cells were recovered on the day of transduction. Thereafter, the cells showed significant expansion, with a mean expansion in the range of about 94±46-fold (range of about 40 to about 153) over the subsequent 8 days, resulting in a net 58±33-fold expansion. Cell expansion in both small- and large-scale experiments was similar, with net expansion of about 45±29 fold (range of about 25 to about 90) in 5 small-scale experiments and about 79±34 fold (range of about 50 to about 116) in 3 large-scale experiments.

### ΔCD19 Enables Efficient and Selective Enrichment of Transduced Cells on Immunomagnetic Columns

The efficiency of suicide gene activation sometimes depends on the functionality of the suicide gene itself, and sometimes on the selection system used to enrich for gene-modified cells. The use of CD19 as a selectable marker was investigated to determine if CD19 selection enabled the selection of gene-modified cells with sufficient purity and yield, and whether selection had any deleterious effects on subsequent cell growth. Small-scale selection was performed according to manufacturer's instruction; however, it was determined that large-scale selection was optimum when 101 of CD19 microbeads was used per 1.3×10⁷ cells. FACS analysis was performed at 24 hours after immunomagnetic selection to minimize interference from anti-CD19 microbeads. The purity of the cells after immunomagnetic selection was consistently greater than 90%: mean percentage of CD19⁺ cells was in the range of about 98.3%±0.5% (n=5) in small-scale selections and in the range of about 97.4%±0.9% (n=3) in large-scale CliniMacs selections

The absolute yield of small- and large-scale selections were about 31%±11% and about 28%±6%, respectively; after correction for transduction efficiency. The mean recovery of transduced cells was about 54%±14% in small-scale and about 72%±18% in large-scale selections. The selection process did not have any discernable deleterious effect on subsequent cell expansion. In 4 experiments, the mean cell expansion over 3 days following CD19 immunomagnetic selection was about 3.5 fold for the CD19 positive fraction versus about 4.1 fold for non-selected transduced cells (p=0.34) and about 3.7 fold for non-transduced cells (p=0.75).

### Immunophenotype of Gene-Modified Allodepleted Cells

The final cell product (gene-modified allodepleted cells that had been cryopreserved 8 days after transduction) was immunophenotyped and was found to contain both CD4 and CD8 cells, with CD8 cells predominant, at 62%±11% CD8⁺ versus 23%±8% CD4⁺, as shown in the table below. NS=not significant, SD=standard deviation.

TABLE 1

| | Unmanipulated | Gene-modified | |
|---|---|---|---|
| | PBMC | allodepleted cells | |
| | (mean % ± SD) | (mean % ± SD) | |
| T cells: Total CD3+ | 82 ± 6 | 95 ± 6 | NS |
| CD3+ 4+ | 54 ± 5 | 23 ± 8 | p < 0.01 |
| CD3+ 8+ | 26 ± 9 | 62 ± 11 | p < 0.001 |
| NK cells: CD3− 56+ | 6 ± 3 | 2 ± 1 | NS |
| Memory phenotype | | | |
| CD45RA+ | 66 ± 3 | 10 ± 5 | p < 0.001 |
| CD45RO+ | 26 ± 2 | 78 ± 7 | p < 0.001 |
| CD45RA− CD62L+ | 19 ± 1 | 24 ± 7 | NS |
| CD45RA− CD62L− | 9 ± 1 | 64 ± 7 | p < 0.001 |
| CD27+ CD28+ | 67 ± 7 | 19 ± 9 | p < 0.001 |
| CD27+ CD28− | 7 ± 3 | 9 ± 4 | NS |
| CD27− CD28+ | 4 ± 1 | 19 ± 8 | p < 0.05 |
| CD27− CD28− | 22 ± 8 | 53 ± 18 | p < 0.05 |

The majorities of cells were CD45RO⁺ and had the surface immunophenotype of effector memory T cells. Expression of memory markers, including CD62L, CD27 and CD28, was heterogeneous. Approximately 24% of cells expressed CD62L, a lymph node-homing molecule predominantly expressed on central memory cells.

### Gene-Modified Allodepleted Cells Retained Antiviral Repertoire and Functionality

The ability of end-product cells to mediate antiviral immunity was assessed by interferon-ELISpot, cytotoxicity assay, and pentamer analysis. The cryopreserved gene-modified allodepleted cells were used in all analyses, since they were representative of the product currently being evaluated for use in a clinical study. Interferon-□ secretion in response to adenovirus, CMV or EBV antigens presented by donor cells was preserved although there was a trend towards reduced anti-EBV response in gene-modified allodepleted cells versus unmanipulated PBMC. The response to viral antigens was assessed by ELISpot in 4 pairs of unmanipulated PBMC and gene-modified allodepleted cells (GMAC). Adenovirus and CMV antigens were presented by donor-derived activated monocytes through infection with Ad5f35 null vector and Ad5f35-pp65 vector, respectively. EBV antigens were presented by donor EBV-LCL. The number of spot-forming units (SFU) was corrected for stimulator- and responder-alone wells. Only three of four donors were evaluable for CMV response, one seronegative donor was excluded.

Cytotoxicity was assessed using donor-derived EBV-LCL as targets. Gene-modified allodepleted cells that had undergone 2 or 3 rounds of stimulation with donor-derived EBV-LCL could efficiently lyse virus-infected autologous target cells Gene-modified allodepleted cells were stimulated with donor EBV-LCL for 2 or 3 cycles. ⁵¹Cr release assay was performed using donor-derived EBV-LCL and donor OKT3 blasts as targets. NK activity was blocked with 30-fold excess cold K562. The left panel shows results from 5 independent experiments using totally or partially mismatched donor-recipient pairs. The right panel shows results from 3 experiments using unrelated HLA haploidentical donor-recipient pairs. Error bars indicate standard deviation.

EBV-LCLs were used as antigen-presenting cells during selective allodepletion, therefore it was possible that EBV-specific T cells could be significantly depleted when the donor and recipient were haploidentical. To investigate this hypothesis, three experiments using unrelated HLA-haploidentical donor-recipient pairs were included, and the results showed that cytotoxicity against donor-derived EBV-LCL was retained. The results were corroborated by pentamer analysis for T cells recognizing HLA-B8-RAKFKQLL (SEQ ID NO: 287), an EBV lytic antigen (BZLF1) epitope, in two informative donors following allodepletion against HLA-B8 negative haploidentical recipients. Unmanipulated PBMC were used as comparators. The RAK-pentamer positive population was retained in gene-modified allodepleted cells and could be expanded following several rounds of in vitro stimulation with donor-derived EBV-LCL. Together, these results indicate that gene-modified allodepleted cells retained significant anti-viral functionality.

### Regulatory T Cells in the Gene-Modified Allodepleted Cell Population

Flow cytometry and functional analysis were used to determine whether regulatory T cells were retained in our allodepleted, gene modified, T cell product. A Foxp3⁺ CD4⁺25⁺ population was found. Following immunomagnetic separation, the CD4⁺CD25⁺ enriched fraction demonstrated suppressor function when co-cultured with CFSE-labeled autologous PBMC in the presence of OKT3 and allogeneic feeders. Donor-derived PBMC was labeled with CFSE and stimulated with OKT3 and allogeneic feeders. CD4⁺CD25⁺ cells were immunomagnetically selected from the gene-modified cell population and added at 1:1 ratio to test wells. Flow cytometry was performed after 5 days. Gene-modified T cells were gated out by CD19 expression. The addition of CD4⁺CD25⁺ gene-modified cells (bottom panel) significantly reduced cell proliferation. Thus, allodepleted T cells may reacquire regulatory phenotype even after exposure to a CD25 depleting immunotoxin.

Gene-Modified Allodepleted Cells were Efficiently and Rapidly Eliminated by Addition of Chemical Inducer of Dimerization

The day following immunomagnetic selection, 10 nM of the chemical inducer of dimerization, AP20187, was added to induce apoptosis, which appeared within 24 hours. FACS analysis with annexin V and 7-AAD staining at 24 hours showed that only about 5.5%±2.5% of AP20187-treated cells remained viable, whereas about 81.0%±9.0% of untreated cells were viable. Killing efficiency after correction for baseline viability was about 92.9%±3.8%. Large-scale CD19 selection produced cells that were killed with similar efficiency as small-scale selection: mean viability with and without AP20187, and percentage killing, in large and small scale were about 3.9%, about 84.0%, about 95.4% (n=3) and about 6.6%, about 79.3%, about 91.4% (n=5) respectively. AP20187 was non-toxic to non-transduced cells: viability with and without AP20187 was about 86%±9% and 87%±8% respectively (n=6).

Transgene Expression and Function Decreased with Extended Culture but were Restored Upon Cell Reactivation

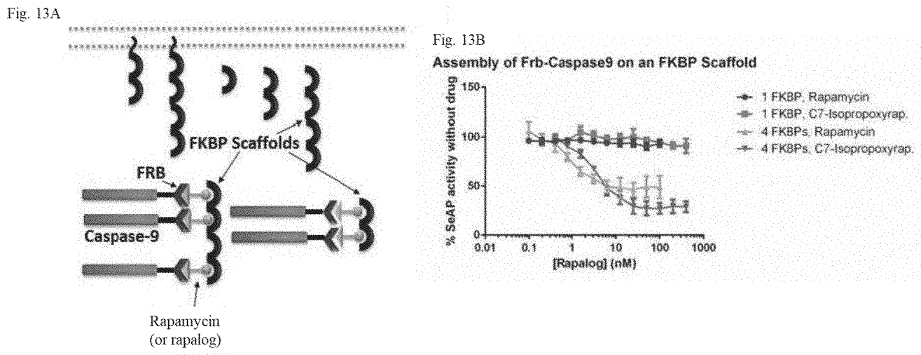

To assess the stability of transgene expression and function, cells were maintained in T cell culture medium and low dose IL-2 (50 U/ml) until 24 days after transduction. A portion of cells was then reactivated with OKT3/anti-CD28. CD19 expression was analyzed by flow cytometry 48 to 72 hours later, and suicide gene function was assessed by treatment with 10 nM AP20187. The obtained are for cells from day 5 post transduction (ie, 1 day after CD 19 selection) and day 24 post transduction, with or without 48-72 hours of reactivation (5 experiments). In 2 experiments, CD25 selection was performed after OKT3/αCD28 activation to further enrich activated cells. Error bars represent standard deviation. * indicates p<0.05 when compared to cells from day 5 post transduction. By day 24, surface CD19 expression fell from about 98%±1% to about 88%±4% (p<0.05) with a parallel decrease in mean fluorescence intensity (MFI) from 793±128 to 478±107 (p<0.05) (see FIG. 13B). Similarly, there was a significant reduction in suicide gene function: residual viability was 19.6±5.6% following treatment with AP20187; after correction for baseline viability of 54.8±20.9%, this equated to killing efficiency of only 63.1±6.2%.

To determine whether the decrease in transgene expression with time was due to reduced transcription following T cell quiescence or to elimination of transduced cells, a portion of cells were reactivated on day 22 post transduction with OKT3 and anti-CD28 antibody. At 48 to 72 hours (day 24 or 25 post transduction), OKT3/αCD28-reactivated cells had significantly higher transgene expression than non-reactivated cells. CD19 expression increased from about 88%±4% to about 93%±4% (p<0.01) and CD19 MFI increased from 478±107 to 643±174 (p<0.01). Additionally, suicide gene function also increased significantly from about a 63.1%±6.2% killing efficiency to about a 84.6%±8.0% (p<0.01) killing efficiency. Furthermore, killing efficiency was completely restored if the cells were immunomagnetically sorted for the activation marker CD25: killing efficiency of CD25 positive cells was about 93%.2±1.2%, which was the same as killing efficiency on day 5 post transduction (93.1±3.5%). Killing of the CD25 negative fraction was 78.6±9.1%.

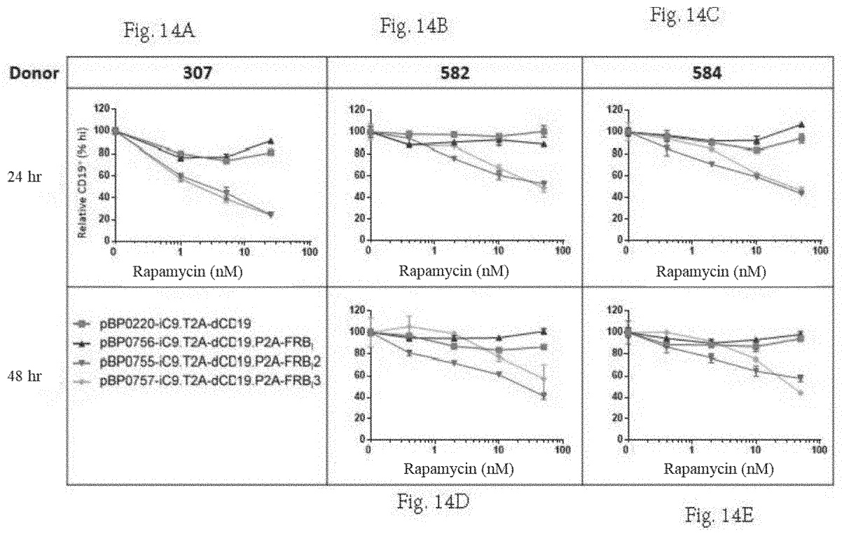

An observation of note was that many virus-specific T cells were spared when dimerizer was used to deplete gene-modified cells that have been re-activated with allogeneic PBMC, rather than by non-specific mitogenic stimuli. After 4 days reactivation with allogeneic cells, as shown in FIGS. 14A and 14B, treatment with AP20187 spares (and thereby enriches) viral reactive subpopulations, as measured by the proportion of T cells reactive with HLA pentamers specific for peptides derived from EBV and CMV. Gene-modified allodepleted cells were maintained in culture for 3 weeks post-transduction to allow transgene down-modulation. Cells were stimulated with allogeneic PBMC for 4 days, following which a portion was treated with 10 nM AP20187. The frequency of EBV-specific T cells and CMV-specific T cells were quantified by pentamer analysis before allostimulation, after allostimulation, and after treatment of allostimulated cells with dimerizer. The percentage of virus-specific T cells decreased after allostimulation. Following treatment with dimerizer, virus-specific T cells were partially and preferentially retained.

### Discussion

The feasibility of engineering allogeneic T cells with two distinct safety mechanisms, selective allodepletion and suicide gene-modification has been demonstrated herein. In combination, these modifications can enhance and/or enable addback of substantial numbers of T cells with anti-viral and anti-tumor activity, even after haploidentical transplantation. The data presented herein show that the suicide gene, iCasp9, functions efficiently (>90% apoptosis after treatment with dimerizer) and that down-modulation of transgene expression that occurred with time was rapidly reversed upon T cell activation, as would occur when alloreactive T cells encountered their targets. Data presented herein also show that CD19 is a suitable selectable marker that enabled efficient and selective enrichment of transduced cells to >90% purity. Furthermore, the data presented herein indicate that these manipulations had no discernable effects on the immunological competence of the engineered T cells with retention of antiviral activity, and regeneration of a CD4⁺CD25⁺ Foxp3⁺ population with Treg activity.

Given that the overall functionality of suicide genes depends on both the suicide gene itself and the marker used to select the transduced cells, translation into clinical use requires optimization of both components, and of the method used to couple expression of the two genes. The two most widely used selectable markers, currently in clinical practice, each have drawbacks. Neomycin phosphotransferase (neo) encodes a potentially immunogenic foreign protein and requires a 7-day culture in selection medium, which not only increases the complexity of the system, but is also potentially damaging to virus-specific T cells. A widely used surface selection marker, LNGFR, has recently had concerns raised, regarding its oncogenic potential and potential correlation with leukemia, in a mouse model, despite its apparent clinical safety. Furthermore, LNGFR selection is not widely available, because it is used almost exclusively in gene therapy. A number of alternative selectable markers have been suggested. CD34 has been well-studied in vitro, but the steps required to optimize a system configured primarily for selection of rare hematopoietic progenitors, and more critically, the potential for altered in vivo T cell homing, make CD34 sub-optimal for use as a selectable marker for a suicide switch expression construct. CD19 was chosen as an alternative selectable marker, since clinical grade CD19 selection is readily available as a method for B-cell depletion of stem cell autografts. The results presented herein demonstrated that CD19 enrichment could be performed with high purity and yield and, furthermore, the selection process had no discernable effect on subsequent cell growth and functionality.

The effectiveness of suicide gene activation in CD19-selected iCasp9 cells compared very favorably to that of neo- or LNGFR-selected cells transduced to express the HSVtk gene. The earlier generations of HSVtk constructs provided 80-90% suppression of ³H-thymidine uptake and showed similar reduction in killing efficiency upon extended in vitro culture, but were nonetheless clinically efficacious. Complete resolution of both acute and chronic GVHD has been reported with as little as 80% in vivo reduction in circulating gene-modified cells. These data support the hypothesis that transgene down-modulation seen in vitro is unlikely to be an issue because activated T cells responsible for GVHD will upregulate suicide gene expression and will therefore be selectively eliminated in vivo. Whether this effect is sufficient to allow retention of virus- and leukemia-specific T cells in vivo will be tested in a clinical setting. By combining in vitro selective allodepletion prior to suicide gene modification, the need to activate the suicide gene mechanism may be significantly reduced, thereby maximizing the benefits of addback T cell based therapies.

The high efficiency of iCasp9-mediated suicide seen in vitro has been replicated in vivo. In a SCID mouse-human xenograft model, more than 99% of iCasp9-modified T cells were eliminated after a single dose of dimerizer. AP1903, which has extremely close functional and chemical equivalence to AP20187, and currently is proposed for use in a clinical application, has been safety tested on healthy human volunteers and shown to be safe. Maximal plasma level of between about 10 ng/ml to about 1275 ng/ml AP1903 (equivalent to between about 7 nM to about 892 nM) was attained over a 0.01 mg/kg to 1.0 mg/kg dose range administered as a 2-hour intravenous infusion. There were substantially no significant adverse effects. After allowing for rapid plasma redistribution, the concentration of dimerizer used in vitro remains readily achievable in vivo.

Optimal culture conditions for maintaining the immunological competence of suicide gene-modified T cells must be determined and defined for each combination of safety switch, selectable marker and cell type, since phenotype, repertoire and functionality can all be affected by the stimulation used for polyclonal T cell activation, the method for selection of transduced cells, and duration of culture. The addition of CD28 co-stimulation and the use of cell-sized paramagnetic beads to generate gene modified-cells that more closely resemble unmanipulated PBMC in terms of CD4:CD8 ratio, and expression of memory subset markers including lymph node homing molecules CD62L and CCR7, may improve the in vivo functionality of gene-modified T cells. CD28 co-stimulation also may increase the efficiency of retroviral transduction and expansion. Interestingly however, the addition of CD28 co-stimulation was found to have no impact on transduction of allodepleted cells, and the degree of cell expansion demonstrated was higher when compared to the anti-CD3 alone arm in other studies. Furthermore, iCasp9-modified allodepleted cells retained significant anti-viral functionality, and approximately one fourth retained CD62L expression. Regeneration of CD4⁺CD25⁺ Foxp3⁺ regulatory T cells was also seen. The allodepleted cells used as the starting material for T cell activation and transduction may have been less sensitive to the addition of anti-CD28 antibody as co-stimulation. CD25-depleted PBMC/EBV-LCL co-cultures contained T cells and B cells that already express CD86 at significantly higher level than unmanipulated PBMCs and may they provide co-stimulation. Depletion of CD25⁺ regulatory T cells prior to polyclonal T cell activation with anti-CD3 has been reported to enhance the immunological competence of the final T cell product. In order to minimize the effect of in vitro culture and expansion on functional competence, a relatively brief culture period was used in some experiments presented herein, whereby cells were expanded for a total of 8 days post-transduction with CD19-selection being performed on day 4.

Finally, scaled up production was demonstrated such that sufficient cell product can be produced to treat adult patients at doses of up to 10⁷ cells/kg: allodepleted cells can be activated and transduced at 4×10⁷ cells per flask, and a minimum of 8-fold return of CD19-selected final cell product can be obtained on day 8 post-transduction, to produce at least 3×10⁸ allodepleted gene-modified cells per original flask. The increased culture volume is readily accommodated in additional flasks or bags.

The allodepletion and iCasp9-modification presented herein may significantly improve the safety of adding back T cells, particularly after haploidentical stem cell allografts. This should in turn enable greater dose-escalation, with a higher chance of producing an anti-leukemia effect.

### Example 3: CASPALLO—Phase 1 Clinical Trial of Allodepleted T Cells Transduced with Inducible Caspase-9 Suicide Gene after Haploidentical Stem Cell Transplantation

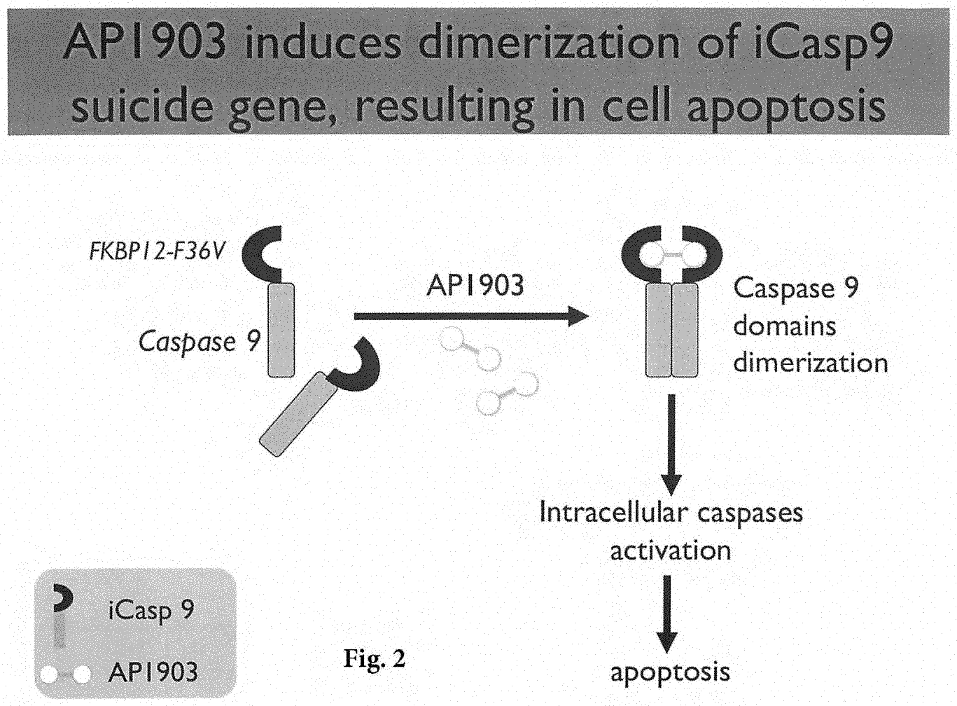

This example presents results of a phase 1 clinical trial using the alternative suicide gene strategy illustrated in FIG. 2. Briefly, donor peripheral blood mononuclear cells were co-cultured with recipient irradiated EBV-transformed lymphoblastoid cells (40:1) for 72 hrs, allodepleted with a CD25 immunotoxin and then transduced with a retroviral supernatant carrying the iCasp9 suicide gene and a selection marker (ΔCD19); ΔCD19 allowed enrichment to >90% purity via immunomagnetic selection.

An example of a protocol for generation of a cell therapy product is provided herein.

### Source Material

Up to 240 ml (in 2 collections) of peripheral blood was obtained from the transplant donor according to established protocols. In some cases, dependent on the size of donor and recipient, a leukopheresis was performed to isolate sufficient T cells. 10 cc-30 cc of blood also was drawn from the recipient and was used to generate the Epstein Barr virus (EBV)-transformed lymphoblastoid cell line used as stimulator cells. In some cases, dependent on the medical history and/or indication of a low B cell count, the LCLs were generated using appropriate 1st degree relative (e.g., parent, sibling, or offspring) peripheral blood mononuclear cells.

### Generation of Allodepleted Cells

Allodepleted cells were generated from the transplant donors as presented herein. Peripheral blood mononuclear cells (PBMCs) from healthy donors were co-cultured with irradiated recipient Epstein Barr virus (EBV)-transformed lymphoblastoid cell lines (LCL) at responder-to-stimulator ratio of 40:1 in serum-free medium (AIM V; Invitrogen, Carlsbad, Calif.). After 72 hours, activated T cells that express CD25 were depleted from the co-culture by overnight incubation in RFT5-SMPT-dgA immunotoxin. Allodepletion is considered adequate if the residual CD3⁺CD25⁺ population was <1% and residual proliferation by ³H-thymidine incorporation was <10%.

### Retroviral Production

A retroviral producer line clone was generated for the iCasp9-CD19 construct. A master cell-bank of the producer also was generated. Testing of the master-cell bank was performed to exclude generation of replication competent retrovirus and infection by Mycoplasma, HIV, HBV, HCV and the like. The producer line was grown to confluency, supernatant harvested, filtered, aliquoted and rapidly frozen and stored at −80° C. Additional testing was performed on all batches of retroviral supernatant to exclude Replication Competent Retrovirus (RCR) and issued with a certificate of analysis, as per protocol.

### Transduction of Allodepleted Cells

Allodepleted T-lymphocytes were transduced using Fibronectin. Plates or bags were coated with recombinant Fibronectin fragment CH-296 (Retronectin™, Takara Shuzo, Otsu, Japan). Virus was attached to retronectin by incubating producer supernatant in coated plates or bags. Cells were then transferred to virus coated plates or bags. After transduction allodepleted T cells were expanded, feeding them with IL-2 twice a week to reach the sufficient number of cells as per protocol.

### CD19 Immunomagnetic Selection

Immunomagnetic selection for CD19 was performed 4 days after transduction. Cells are labeled with paramagnetic microbeads conjugated to monoclonal mouse anti-human CD19 antibodies (Miltenyi Biotech, Auburn, Calif.) and selected on a CliniMacs Plus automated selection device. Depending upon the number of cells required for clinical infusion cells were either cryopreserved after the CliniMacs selection or further expanded with IL-2 and cryopreserved on day 6 or day 8 post transduction.

### Freezing

Aliquots of cells were removed for testing of transduction efficiency, identity, phenotype and microbiological culture as required for final release testing by the FDA. The cells were cryopreserved prior to administration according to protocol.

### Study Drugs

### RFT5-SMPT-dgA

RFT5-SMPT-dgA is a murine IgG1 anti-CD25 (IL-2 receptor alpha chain) conjugated via a hetero-bifunctional crosslinker [N-succinimidyloxycarbonyl-alpha-methyl-d-(2-pyridylthio) toluene] (SMPT) to chemically deglycosylated ricin A chain (dgA). RFT5-SMPT-dgA is formulated as a sterile solution at 0.5 mg/ml.

### Synthetic Homodimerizer, AP1903

Mechanism of Action: AP1903-inducible cell death is achieved by expressing a chimeric protein comprising the intracellular portion of the human (Caspase-9 protein) receptor, which signals apoptotic cell death, fused to a drug-binding domain derived from human FK506-binding protein (FKBP). This chimeric protein remains quiescent inside cells until administration of AP1903, which cross-links the FKBP domains, initiating Caspase signaling and apoptosis.

Toxicology: AP1903 has been evaluated as an Investigational New Drug (IND) by the FDA and has successfully completed a phase 1 clinical safety study. No significant adverse effects were noted when API 903 was administered over a 0.01 mg/kg to 1.0 mg/kg dose range.

Pharmacology/Pharmacokinetics: Patients received 0.4 mg/kg of AP1903 as a 2 h infusion-based on published Pk data which show plasma concentrations of 10 ng/mL-I275 ng/mL over the 0.01 mg/kg to 1.0 mg/kg dose range with plasma levels falling to 18% and 7% of maximum at 0.5 and 2 hrs post dose.

Side Effect Profile in Humans: No serious adverse events occurred during the Phase 1 study in volunteers. The incidence of adverse events was very low following each treatment, with all adverse events being mild in severity. Only one adverse event was considered possibly related to AP1903. This was an episode of vasodilatation, presented as “facial flushing” for 1 volunteer at the 1.0 mg/kg AP1903 dosage. This event occurred at 3 minutes after the start of infusion and resolved after 32 minutes duration. All other adverse events reported during the study were considered by the investigator to be unrelated or to have improbable relationship to the study drug. These events included chest pain, flu syndrome, halitosis, headache, injection site pain, vasodilatation, increased cough, rhinitis, rash, gum hemorrhage, and ecchymosis.

Patients developing grade 1 GVHD were treated with 0.4 mg/kg AP1903 as a 2-hour infusion. Protocols for administration of AP1903 to patients grade 1 GVHD were established as follows. Patients developing GvHD after infusion of allodepleted T cells are biopsied to confirm the diagnosis and receive 0.4 mg/kg of AP1903 as a 2 h infusion. Patients with Grade I GVHD received no other therapy initially, however if they showed progression of GvHD conventional GvHD therapy was administered as per institutional guidelines. Patients developing grades 2-4 GVHD were administered standard systemic immunosuppressive therapy per institutional guidelines, in addition to the AP1903 dimerizer drug.

Instructions for preparation and infusion: AP1903 for injection is obtained as a concentrated solution of 2.33 ml in a 3-ml vial, at a concentration of 5 mg/ml, (i.e., 11.66 mg per vial). AP1903 may also be provided, for example, at 8 ml per vial, at 5 mg/ml. Prior to administration, the calculated dose was diluted to 100 mL in 0.9% normal saline for infusion. AP1903 for injection (0.4 mg/kg) in a volume of 100 ml was administered via IV infusion over 2 hours, using a non-DEHP, non-ethylene oxide sterilized infusion set and infusion pump.

The iCasp9 suicide gene expression construct (e.g., SFG.iCasp9.2A.ΔCD19), shown in FIG. 24 consists of inducible Caspase-9 (iCasp9) linked, via a cleavable 2A-like sequence, to truncated human CD19 (ΔCD19). iCasp9 includes a human FK506-binding protein (FKBP12; GenBank AH002 818) with an F36V mutation, connected via a Ser-Gly-Gly-Gly-Ser-Gly linker (SEQ ID NO: 289) to human Caspase-9 (CASP9; GenBank NM 001229). The F36V mutation may increase the binding affinity of FKBP12 to the synthetic homodimerizer, AP20187 or AP1903. The Caspase recruitment domain (CARD) has been deleted from the human Caspase-9 sequence and its physiological function has been replaced by FKBP12. The replacement of CARD with FKBP12 increases transgene expression and function. The 2A-like sequence encodes an 18 amino acid peptide from Thosea Asigna insect virus, which mediates >99% cleavage between a glycine and terminal proline residue, resulting in 17 extra amino acids in the C terminus of iCasp9, and one extra proline residue in the N terminus of CD19. ΔCD19 consists of full length CD19 (GenBank NM 001770) truncated at amino acid 333 (TDPTRRF (SEQ ID NO: 290)), which shortens the intracytoplasmic domain from 242 to 19 amino acids, and removes all conserved tyrosine residues that are potential sites for phosphorylation.

### In Vivo Studies

Three patients received iCasp9⁺ T cells after haplo-CD34⁺ stem cell transplantation (SCT), at dose levels between about 1×10⁶ to about 3×10⁶ cells/kg.

TABLE 2
Characteristics of the patients and clinical outcome.

| | | | | Days | Number | | |
|---|---|---|---|---|---|---|---|
| | | | Disease | from SCT | of cells | | |
| Patient | Sex (age | | status at | to T-cell | infused | Acute | Clinical |
| # | (yr)) | Diagnosis | SCT | infusion | per kg | GvHD | outcome |
| P1 | M(3) | MDS/AML | CR2 | 63 | 1 × 106 | Grade1/2 | Alive in |
| | | | | | | (skin, | CR >12 |
| | | | | | | liver) | months |
| | | | | | | | No GvHD |
| P2 | F(17) | B-ALL | CR2 | 80 and | (1 × 106)2 | Grade 1 | Alive in |
| | | | | 112 | | (skin) | CR >12 |
| | | | | | | | months |
| | | | | | | | No GvHD |
| P3 | M(8) | T-ALL | PIF/CR1 | 93 | 3 × 106 | None | Alive in |
| | | | | | | | CR >12 |
| | | | | | | | No GvHD |
| P4 | F(4) | T-ALL | Active | 30 | 3 × 106 | Grade 1 | Alive in |
| | | | disease | | | (skin) | CR >12 |
| | | | | | | | No GvHD |

Infused T cells were detected in vivo by flow cytometry (CD3⁺ΔCD19⁺) or qPCR as early as day 7 after infusion, with a maximum fold expansion of 170±5 (day 29±9 after infusion), as illustrated in FIGS. 27, 28, and 29. Two patients developed grade I/II aGVHD (see FIGS. 31-32) and AP1903 administration caused <90% ablation of CD3⁺ΔCD19⁺ cells, within 30 minutes of infusion (see FIGS. 30, 33, and 34), with a further log reduction within 24 hours, and resolution of skin and liver aGvHD within 24 hrs, showing that iCasp9 transgene was functional in vivo. For patient two, the disappearance of skin rash within 24 hours post treatment was observed.

TABLE 3
Patients with GvHD (dose level 1)

| | SCT to GvHD | T cells to GvHD | |
|---|---|---|---|
| Patient | (days) | (days) | GvHD (grade/site) |
| 1 | 77 | 14 | 2 (liver, skin) |
| 2 | 124 | 45/13 | 2 (skin) |

Ex vivo experiments confirmed this data. Furthermore, the residual allodepleted T cells were able to expand and were reactive to viruses (CMV) and fungi (Aspergillus fumigatus) (IFN-γ production). These in vivo studies found that a single dose of dimerizer drug can reduce or eliminate the subpopulation of T cells causing GvHD, but can spare virus specific CTLs, which can then re-expand.

### Immune Reconstitution

Depending on availability of patient cells and reagents, immune reconstitution studies (Immunophenotyping, T and B cell function) may be obtained at serial intervals after transplant. Several parameters measuring immune reconstitution resulting from iCaspase transduced allodepleted T cells will be analyzed. The analysis includes repeated measurements of total lymphocyte counts, T and CD19 B cell numbers, and FACS analysis of T cell subsets (CD3, CD4, CD8, CD16, CD19, CD27, CD28, CD44, CD62L, CCR7, CD56, CD45RA, CD45RO, alpha/beta and gamma/delta T cell receptors). Depending on the availability of a patient's T cells, T regulatory cell markers such as CD41, CD251, and FoxP3 also are analyzed. Approximately 10-60 ml of patient blood is taken, when possible, 4 hours after infusion, weekly for 1 month, monthly×9 months, and then at 1 and 2 years. The amount of blood taken is dependent on the size of the recipient and does not exceed 1-2 cc/kg in total (allowing for blood taken for clinical care and study evaluation) at any one blood draw.

### Persistence and Safety of Transduced Allodepleted T Cells

The following analysis was also performed on the peripheral blood samples to monitor function, persistence and safety of transduced T-cells at time-points indicated in the study calendar:

Phenotype by flow cytometry to detect the presence of transgenic cells.

RCR testing by PCR.

Quantitative real-time PCR for detecting retroviral integrants.

RCR testing by PCR is performed pre study, at 3, 6, and 12 months, and then yearly for a total of 15 years. Tissue, cell, and serum samples are archived for use in future studies for RCR as required by the FDA.

### Statistical Analysis and Stopping Rules.

The MTD is defined to be the dose which causes grade III/IV acute GVHD in at most 25% of eligible cases. The determination is based on a modified continual reassessment method (CRM) using a logistic model with a cohort of size 2. Three dose groups are being evaluated namely, 1×10⁶, 3×10⁶, 1×10⁷ with prior probabilities of toxicity estimated at 10%, 15%, and 30%, respectively. The proposed CRM design employs modifications to the original CRM by accruing more than one subject in each cohort, limiting dose escalation to no more than one dose level, and starting patient enrollment at the lowest dose level shown to be safe for non-transduced cells. Toxicity outcome in the lowest dose cohort is used to update the dose-toxicity curve. The next patient cohort is assigned to the dose level with an associated probability of toxicity closest to the target probability of 25%. This process continues until at least 10 patients have been accrued into this dose-escalation study. Depending on patient availability, at most 18 patients may be enrolled into the Phase 1 trial or until 6 patients have been treated at the current MTD.

The final MTD will be the dose with probability closest to the target toxicity rate at these termination points.

Simulations were performed to determine the operating characteristics of the proposed design and compared this with a standard 3+3 dose-escalation design. The proposed design delivers better estimates of the MTD based on a higher probability of declaring the appropriate dose level as the MTD, afforded smaller number of patients accrued at lower and likely ineffective dose levels, and maintained a lower average total number of patients required for the trial. A shallow dose-toxicity curve is expected over the range of doses proposed herein and therefore accelerated dose-escalations can be conducted without comprising patient safety. The simulations performed indicate that the modified CRM design does not incur a larger average number of total toxicities when compared to the standard design (total toxicities equal to 1.9 and 2.1, respectively.).

Grade III/IV GVHD that occurs within 45 days after initial infusion of allodepleted T cells will be factored into the CRM calculations to determine the recommended dose for the subsequent cohort. Real-time monitoring of patient toxicity outcome is performed during the study in order to implement estimation of the dose-toxicity curve and determine dose level for the next patient cohort using one of the pre-specified dose levels.

Treatment limiting toxicities will include:

grade 4 reactions related to infusion,
graft failure (defined as a subsequent decline in the ANC to <500/mm³ for three consecutive measurements on different days, unresponsive to growth factor therapy that persists for at least 14 days.) occurring within 30 days after infusion of TC-T
grade 4 nonhematologic and noninfectious adverse events, occurring within 30 days after infusion grades 3-4 acute GVHD by 45 days after infusion of TC-T
treatment-related death occurring within 30 days after infusion

GVHD rates are summarized using descriptive statistics along with other measures of safety and toxicity. Likewise, descriptive statistics will be calculated to summarize the clinical and biologic response in patients who receive AP1903 due to great than Grade 1 GVHD.

Several parameters measuring immune reconstitution resulting from iCaspase transduced allodepleted T cells will be analyzed. These include repeated measurements of total lymphocyte counts, T and CD19 B cell numbers, and FACS analysis of T cell subsets (CD3, CD4, CDS, CD16, CD19, CD27, CD44, CD62L, CCR7, CD56, CD45RA, CD45RO, alpha/beta and gamma/delta T cell receptors). If sufficient T cells remain for analysis, T regulatory cell markers such as CD4/CD25/FoxP3 will also be analyzed. Each subject will be measured pre-infusion and at multiple time points post-infusion as presented above.

Descriptive summaries of these parameters in the overall patient group and by dose group as well as by time of measurement will be presented. Growth curves representing measurements over time within a patient will be generated to visualize general patterns of immune reconstitution. The proportion of iCasp9 positive cells will also be summarized at each time point. Pairwise comparisons of changes in these endpoints over time compared to pre-infusion will be implemented using paired t-tests or Wilcoxon signed-ranks test.

Longitudinal analysis of each repeatedly-measured immune reconstitution parameter using the random coefficients model will be performed. Longitudinal analysis allows construction of model patterns of immune reconstitution per patient while allowing for varying intercepts and slopes within a patient. Dose level as an independent variable in the model to account for the different dose levels received by the patients will also be used. Testing whether there is a significant improvement in immune function over time and estimates of the magnitude of these improvements based on estimates of slopes and its standard error will be possible using the model presented herein. Evaluation of any indication of differences in rates of immune reconstitution across different dose levels of CTLs will also be performed. The normal distribution with an identity link will be utilized in these models and implemented using SAS MIXED procedure. The normality assumption of the immune reconstitution parameters will be assessed and transformations (e.g. log, square root) can be performed, if necessary to achieve normality.

A strategy similar to the one presented above can be employed to assess kinetics of T cell survival, expansion and persistence. The ratio of the absolute T cell numbers with the number of marker gene positive cells will be determined and modeled longitudinally over time. A positive estimate of the slope will indicate increasing contribution of T cells for immune recovery. Virus-specific immunity of the iCasp9 T cells will be evaluated by analysis of the number of T cells releasing IFN gamma based on ex-vivo stimulation virus-specific CTLs using longitudinal models. Separate models will be generated for analysis of EBV, CMV and adenovirus evaluations of immunity.

Finally, overall and disease-free survival in the entire patient cohort will be summarized using the Kaplan-Meier product-limit method. The proportion of patients surviving and who are disease-free at 100 days and 1 year post-transplant can be estimated from the Kaplan-Meier curves.

In conclusion, addback of iCasp9⁺ allodepleted T cells after haplo CD34⁺ SCT allows a significant expansion of functional donor lymphocytes in vivo and a rapid clearance of alloreactive T cells with resolution of aGvHD.

### Example 4: In Vivo T Cell Allodepletion

The protocols provided in Examples 1-3 may also be modified to provide for in vivo T cell allodepletion. To extend the approach to a larger group of subjects who might benefit from immune reconstitution without acute GvHD, the protocol may be simplified, by providing for an in vivo method of T cell depletion. In the pre-treatment allodepletion method, as discussed herein, EBV-transformed lymphoblastoid cell lines are first prepared from the recipient, which then act as alloantigen presenting cells. This procedure can take up to 8 weeks, and may fail in extensively pre-treated subjects with malignancy, particularly if they have received rituximab as a component of their initial therapy. Subsequently, the donor T cells are co-cultured with recipient EBV-LCL, and the alloreactive T cells (which express the activation antigen CD25) are then treated with CD25-ricin conjugated monoclonal antibody. This procedure may take many additional days of laboratory work for each subject.

The process may be simplified by using an in vivo method of allodepletion, building on the observed rapid in vivo depletion of alloreactive T cells by dimerizer drug and the sparing of unstimulated but virus/fungus reactive T cells.

If there is development of Grade I or greater acute GvHD, a single dose of dimerizer drug is administered, for example at a dose of 0.4 mg/kg of AP1903 as a 2-hour intravenous infusion. Up to 3 additional doses of dimerizer drug may be administered at 48 hour intervals if acute GvHD persists. In subjects with Grade II or greater acute GvHD, these additional doses of dimerizer drug may be combined with steroids. For patients with persistent GVHD who cannot receive additional doses of the dimerizer due to a Grade III or IV reaction to the dimerizer, the patient may be treated with steroids alone, after either 0 or 1 doses of the dimerizer.

### Generation of Therapeutic T Cells

Up to 240 ml (in 2 collections) of peripheral blood is obtained from the transplant donor according to the procurement consent. If necessary, a leukapheresis is used to obtain sufficient T cells; (either prior to stem cell mobilization or seven days after the last dose of G-CSF). An extra 10-30 mls of blood may also be collected to test for infectious diseases such as hepatitis and HIV.

Peripheral blood mononuclear cells are be activated using anti-human CD3 antibody (e.g. from Orthotech or Miltenyi) on day 0 and expanded in the presence of recombinant human interleukin-2 (rhIL-2) on day 2. CD3 antibody-activated T cells are transduced by the iCaspase-9 retroviral vector on flasks or plates coated with recombinant Fibronectin fragment CH-296 (Retronectin™, Takara Shuzo, Otsu, Japan). Virus is attached to retronectin by incubating producer supernatant in retronectin coated plates or flasks. Cells are then transferred to virus coated tissue culture devices. After transduction T cells are expanded by feeding them with rhIL-2 twice a week to reach the sufficient number of cells as per protocol.

To ensure that the majority of infused T cells carry the suicide gene, a selectable marker, truncated human CD19 (ΔCD19) and a commercial selection device, may be used to select the transduced cells to >90% purity. Immunomagnetic selection for CD19 may be performed 4 days after transduction. Cells are labeled with paramagnetic microbeads conjugated to monoclonal mouse anti-human CD19 antibodies (Miltenyi Biotech, Auburn, Calif.) and selected on a CliniMacs Plus automated selection device. Depending upon the number of cells required for clinical infusion cells might either be cryopreserved after the CliniMacs selection or further expanded with IL-2 and cryopreserved as soon as sufficient cells have expanded (up to day 14 from product initiation).

Aliquots of cells may be removed for testing of transduction efficiency, identity, phenotype, autonomous growth and microbiological examination as required for final release testing by the FDA. The cells are cryopreserved prior to administration.

### Administration of T Cells

The transduced T cells are administered to patients from, for example, between 30 and 120 days following stem cell transplantation. The cryopreserved T cells are thawed and infused through a catheter line with normal saline. For children, premedications are dosed by weight. Doses of cells may range from, for example, from about 1×10⁴ cells/kg to 1×10⁸ cells/kg, for example from about 1×10⁵ cells/kg to 1×10⁷ cells/kg, from about 1×10⁶ cells/kg to 5×10⁶ cells/kg, from about 1×10⁴ cells/kg to 5×10⁶ cells/kg, for example, about 1×10⁴, about 1×10⁵, about 2×10⁵, about 3×10⁵, about 5×10⁵, 6×10⁵, about 7×10⁵, about 8×10⁵, about 9×10⁵, about 1×10⁶, about 2×10⁶, about 3×10⁶, about 4×10⁶, or about 5×10⁶ cells/kg.

### Treatment of GvHD

Patients who develop grade acute GVHD are treated with 0.4 mg/kg AP1903 as a 2-hour infusion. AP1903 for injection may be provided, for example, as a concentrated solution of 2.33 ml in a 3 ml vial, at a concentration of 5 mg/ml, (i.e 11.66 mg per vial). AP1903 may also provided in different sized vials, for example, 8 ml at 5 mg/ml may be provided. Prior to administration, the calculated dose will be diluted to 100 mL in 0.9% normal saline for infusion. AP1903 for Injection (0.4 mg/kg) in a volume of 100 ml may be administered via IV infusion over 2 hours, using a non-DEHP, non-ethylene oxide sterilized infusion set and an infusion pump.

TABLE 4
Sample treatment schedule

| Time | Donor | Recipient |
|---|---|---|
| Pre-transplant | Obtain up to 240 of blood or | |
| | unstimulated leukapheresis | |
| | from bone marrow transplant | |
| | donor. Prepare T cells and | |
| | donor LCLs for later immune | |
| | reconstitution studies. | |
| Day 0 | Anti-CD3 activation of PBMC | |
| Day 2 | IL-2 feed | |
| Day 3 | Transduction | |
| Day 4 | Expansion | |
| Day 6 | CD19 selection. | |
| | Cryopreservation (*if required | |
| | dose is met) | |
| Day 8 | Assess transduction | |
| | efficiency and iCaspase9 | |
| | transgene functionality by | |
| | phenotype. Cryopreservation | |
| | (*if not yet performed) | |
| Day 10 or Day 12 to Day 14 | Cryopreservation (if not yet | |
| | performed) | |
| From 30 to 120 days post- | | Thaw and infuse T cells 30 to |
| transplant | | 120 days post-stem cell |
| | | infusion. |

Other methods may be followed for clinical therapy and assessment as provided in, for example, Examples 1-3 herein.

### Example 5: Using the iCasp9 Suicide Gene to Improve the Safety of Mesenchymal Stromal Cell Therapies

Mesenchymal stromal cells (MSCs) have been infused into hundreds of patients to date with minimal reported deleterious side effects. The long term side effects are not known due to limited follow-up and a relatively short time since MSCs have been used in treatment of disease. Several animal models have indicated that there exists the potential for side effects, and therefore a system allowing control over the growth and survival of MSCs used therapeutically is desirable. The inducible Caspase-9 suicide switch expression vector construct presented herein was investigated as a method of eliminating MSC's in vivo and in vitro.

### Materials and Methods

### MSC Isolation

MSCs were isolated from healthy donors. Briefly, post-infusion discarded healthy donor bone marrow collection bags and filters were washed with RPMI 1640 (HyClone, Logan, Utah) and plated on tissue culture flasks in DMEM (Invitrogen, Carlsbad, Calif.) with 10% fetal bovine serum (FBS), 2 mM alanyl-glutamine (Glutamax, Invitrogen), 100 units/mL penicillin and 100 μg/mL streptomycin (Invitrogen). After 48 hours, the supernatant was discarded and the cells were cultured in complete culture medium (CCM): α-MEM (Invitrogen) with 16.5% FBS, 2 mM alanyl-glutamine, 100 units/mL penicillin and 100 μg/mL streptomycin. Cells were grown to less then 80% confluence and replated at lower densities as appropriate.

### Immunophenotyping

Phycoerythrin (PE), fluorescein isothiocyanate (FITC), peridinin chlorophyll protein (PerCP) or allophycocyanin (APC)-conjugated CD14, CD34, CD45, CD73, CD90, CD105 and CD133 monoclonal antibodies were used to stain MSCs. All antibodies were from Becton Dickinson-Pharmingen (San Diego, Calif.), except where indicated. Control samples labeled with an appropriate isotype-matched antibody were included in each experiment. Cells were analyzed by fluorescence-activated cell sorting FACScan (Becton Dickinson) equipped with a filter set for 4 fluorescence signals.

### Differentiation Studies In Vitro

Adipocytic differentiation. MSCs (7.5×10⁴ cells) were plated in wells of 6-well plates in NH AdipoDiff Medium (Miltenyi Biotech, Auburn, Calif.). Medium was changed every third day for 21 days. Cells were stained with Oil Red 0 solution (obtained by diluting 0.5% w/v Oil Red 0 in isopropanol with water at a 3:2 ratio), after fixation with 4% formaldehyde in phosphate buffered saline (PBS).

Osteogenic differentiation. MSCs (4.5×10⁴ cells) were plated in 6-well plates in NH OsteoDiff Medium (Miltenyi Biotech). Medium was changed every third day for 10 days. Cells were stained for alkaline phosphatase activity using Sigma Fast BCIP/NBT substrate (Sigma-Aldrich, St. Louis, Mo.) as per manufacturer instructions, after fixation with cold methanol.

Chondroblastic differentiation. MSC pellets containing 2.5×10⁵ to 5×10⁵ cells were obtained by centrifugation in 15 mL or 1.5 mL polypropylene conical tubes and cultured in NH ChondroDiff Medium (Miltenyi Biotech). Medium was changed every third day for a total of 24 days. Cell pellets were fixed in 4% formalin in PBS and processed for routine paraffin sectioning. Sections were stained with alcian blue or using indirect immunofluorescence for type II collagen (mouse anti-collagen type II monoclonal antibody MAB8887, Millipore, Billerica, Mass.) after antigen retrieval with pepsin (Thermo Scientific, Fremont, Calif.).

iCasp9-ΔCD19 Retrovirus Production and Transduction of MSCs

The SFG.iCasp9.2A.ΔCD19 (iCasp-ΔCD19) retrovirus consists of iCasp9 linked, via a cleavable 2A-like sequence, to truncated human CD19 (ΔCD19). As noted above, iCasp9 is a human FK506-binding protein (FKBP12) with an F36V mutation, which increases the binding affinity of the protein to a synthetic homodimerizer (AP20187 or AP1903), connected via a Ser-Gly-Gly-Gly-Ser-Gly linker (SEQ ID NO: 289) to human Caspase-9, whose recruitment domain (CARD) has been deleted, its function replaced by FKBP12.

The 2A-like sequence encodes a 20 amino acid peptide from Thosea Asigna insect virus, which mediates more than 99% cleavage between a glycine and terminal proline residue, to ensure separation of iCasp9 and ΔCD19 upon translation. ΔCD19 consists of human CD19 truncated at amino acid 333, which removes all conserved intracytoplasmic tyrosine residues that are potential sites for phosphorylation. A stable PG13 clone producing Gibbon ape leukemia virus (Gal-V) pseudotyped retrovirus was made by transiently transfecting Phoenix Eco cell line (ATCC product #5D3444; ATCC, Manassas, Va.) with SFG.iCasp9.2A.ΔCD19, which yielded Eco-pseudotyped retrovirus. The PG13 packaging cell line (ATCC) was transduced 3 times with Eco-pseudotyped retrovirus to generate a producer line that contained multiple SFG.iCasp9.2A.ΔCD19 proviral integrants per cell. Single-cell cloning was performed, and the PG13 clone that produced the highest titer was expanded and used for vector production. Retroviral supernatant was obtained via culture of the producer cell lines in IMDM (Invitrogen) with 10% FBS, 2 mM alanyl-glutamine, 100 units/mL penicillin and 100 μg/mL streptomycin. Supernatant containing the retrovirus was collected 48 and 72 hours after initial culture. For transduction, approximately 2×10⁴ MSCs/cm² were plated in CM in 6-well plates, T75 or T175 flasks. After 24 hours, medium was replaced by viral supernatant diluted 10-fold together with polybrene (final concentration 5 μg/mL) and the cells were incubated at 37° C. in 5% CO₂ for 48 hours, after which cells were maintained in complete medium.

### Cell Enrichment

For inducible iCasp9-ΔCD19-positive MSC selection for in vitro experiments, retrovirally transduced MSC were enriched for CD19-positive cells using magnetic beads (Miltenyi Biotec) conjugated with anti-CD19 (clone 4G7), per manufacturer instructions. Cell samples were stained with PE- or APC-conjugated CD19 (clone SJ25C1) antibody to assess the purity of the cellular fractions.

### Apoptosis Studies In Vitro

Undifferentiated MSCs. The chemical inducer of dimerization (CID) (AP20187; ARIAD Pharmaceuticals, Cambridge, Mass.) was added at 50 nM to iCasp9-transduced MSCs cultures in complete medium. Apoptosis was evaluated 24 hours later by FACS analysis, after cell harvest and staining with annexin V-PE and 7-AAD in annexin V binding buffer (BD Biosciences, San Diego, Calif.). Control iCasp9-transduced MSCs were maintained in culture without exposure to CID.

Differentiated MSCs. Transduced MSCs were differentiated as presented above. At the end of the differentiation period, CID was added to the differentiation media at 50 nM. Cells were stained appropriately for the tissue being studied, as presented above, and a contrast stain (methylene azure or methylene blue) was used to evaluate the nuclear and cytoplasmic morphology. In parallel, tissues were processed for terminal deoxynucleotidyl-transferase dUTP nick end labeling (TUNEL) assay as per manufacturer instructions (In Situ Cell Death Detection Kit, Roche Diagnostics, Mannheim, Germany). For each time point, four random fields were photographed at a final magnification of 40× and the images were analyzed with ImageJ software version 1.430 (NIH, Bethesda, Md.). Cell density was calculated as the number of nuclei (DAPI positivity) per unit of surface area (in mm²). The percentage of apoptotic cells was determined as the ratio of the number of nuclei with positive TUNEL signal (FITC positivity) to the total number of nuclei. Controls were maintained in culture without CID.

### In Vivo Killing Studies in Murine Model

All mouse experiments were performed in accordance with the Baylor College of Medicine animal husbandry guidelines. To assess the persistence of modified MSCs in vivo, a SCID mouse model was used in conjunction with an in vivo imaging system. MSCs were transduced with retroviruses coding for the enhanced green fluorescent protein-firefly luciferase (eGFP-FFLuc) gene alone or together with the iCasp9-ΔCD19 gene. Cells were sorted for eGFP positivity by fluorescence activated cell sorting using a MoFlo flow cytometer (Beckman Coulter, Fullerton, Calif.). Doubly transduced cells were also stained with PE-conjugated anti-CD19 and sorted for PE-positivity. SCID mice (8-10 weeks old) were injected subcutaneously with 5×10⁵ MSCs with and without iCasp9-ΔCD19 in opposite flanks. Mice received two intraperitoneal injections of 50 μg of CID 24 hours apart starting a week later. For in vivo imaging of MSCs expressing eGFP-FFLuc, mice were injected intraperitoneally with D-luciferin (150 mg/kg) and analyzed using the Xenogen-IVIS Imaging System. Total luminescence (a measurement proportional to the total labeled MSCs deposited) at each time point was calculated by automatically defining regions-of-interest (ROIs) over the MSC implantation sites. These ROIs included all areas with luminescence signals at least 5% above background. Total photon counts were integrated for each ROI and an average value calculated. Results were normalized so that time zero would correspond to 100% signal.

In a second set of experiments, a mixture of 2.5×10⁶ eGFP-FFLuc-labeled MSCs and 2.5×10⁶ eGFP-FFLuc-labeled, iCasp9-ΔCD19-transduced MSCs was injected subcutaneously in the right flank, and the mice received two intraperitoneal injections of 50 μg of CID 24 h apart starting 7 days later. At several time points after CID injection, the subcutaneous pellet of MSCs was harvested using tissue luminescence to identify and collect the whole human specimen and to minimize mouse tissue contamination. Genomic DNA was then isolated using QIAmp® DNA Mini (Qiagen, Valencia, Calif.). Aliquots of 100 ng of DNA were used in a quantitative PCR (qPCR) to determine the number of copies of each transgene using specific primers and probes (for the eGFP-FFLuc construct: forward primer 5′-TCCGCCCTGAGCAAAGAC-3′ (SEQ ID NO: 291), reverse 5′-ACGAACTCCAGCAGGACCAT-3′ (SEQ ID NO: 292), probe 5′ FAM, 6-carboxyfluorescein-ACGAGAAGCGCGATC-3′ MGBNFQ (SEQ ID NO: 293), minor groove binding non-fluorescent quencher; iCasp9-ΔCD19: forward 5′-CTGGAATCTGGCGGTGGAT-3′ (SEQ ID NO: 294), reverse 5′-CAAACTCTCAAGAGCACCGACAT-3′ (SEQ ID NO: 295), probe 5′ FAM-CGGAGTCGACGGATT-3′ MGBNFQ (SEQ ID NO: 296)). Known numbers of plasmids containing single copies of each transgene were used to establish standard curves. It was determined that approximately 100 ng of DNA isolated from “pure” populations of singly eGFP-FFLuc- or doubly eGFP-FFLuc- and iCasp9-transduced MSCs had similar numbers of eGFP-FFLuc gene copies (approximately 3.0×10⁴), as well as zero and 1.7×10³ of iCasp9-ΔCD19 gene copies, respectively.

Untransduced human cells and mouse tissues had zero copies of either gene in 100 ng of genomic DNA. Because the copy number of the eGFP gene is the same on identical amounts of DNA isolated from either population of MSCs (iCasp9-negative or positive), the copy number of this gene in DNA isolated from any mixture of cells will be proportional to the total number of eGFP-FFLuc-positive cells (iCasp9-positive plus negative MSCs). Moreover, because iCasp9-negative tissues do not contribute to the iCasp9 copy number, the copy number of the iCasp9 gene in any DNA sample will be proportional to the total number of iCasp9-positive cells. Therefore, if G is the total number of GFP-positive and iCasp9-negative cells and C the total number of GFP-positive and iCasp9-positive cells, for any DNA sample then N_{eGFP}=g·(C+G) and N_{iCasp9}=k·C, where N represents gene copy number and g and k are constants relating copy number and cell number for the eGFP and iCasp9 genes, respectively. Thus N_{iCasp9}/N_{eGFP}=(k/g)·[C/(C+G)], i.e., the ratio between iCasp9 copy number and eGFP copy number is proportional to the fraction of doubly transduced (iCasp9-positive) cells among all eGFP positive cells. Although the absolute values of N_{iCasp9} and N_{eGFP} will decrease with increasing contamination by murine cells in each MSC explant, for each time point the ratio will be constant regardless of the amount of murine tissue included, since both types of human cells are physically mixed. Assuming similar rates of spontaneous apoptosis in both populations (as documented by in vitro culture) the quotient between N_{iCasp9}/N_{eGFP} at any time point and that at time zero will represent the percentage of surviving iCasp9-positive cells after exposure to CID. All copy number determinations were done in triplicate.

### Statistical Analysis

Paired 2-tailed Student's t-test was used to determine the statistical significance of differences between samples. All numerical data are represented as mean±1 standard deviation.

### Results

MSCs are Readily Transduced with iCasp9-ΔCD19 and Maintain their Basic Phenotype

Flow cytometric analysis of MSCs from 3 healthy donors showed they were uniformly positive for CD73, CD90 and CD105 and negative for the hematopoietic markers CD45, CD14, CD133 and CD34. The mononuclear adherent fraction isolated from bone marrow was homogenously positive for CD73, CD90 and CD105 and negative for hematopoietic markers. The differentiation potential, of isolated MSCs, into adipocytes, osteoblasts and chondroblasts was confirmed in specific assays, demonstrating that these cells are bona fide MSCs.

Early passage MSCs were transduced with an iCasp9-ΔCD19 retroviral vector, encoding an inducible form of Caspase-9. Under optimal single transduction conditions, 47±6% of the cells expressed CD19, a truncated form of which is transcribed in cis with iCasp9, serving as a surrogate for successful transduction and allowing selection of transduced cells. The percentage of cells positive for CD19 was stable for more than two weeks in culture, suggesting no deleterious or growth advantageous effects of the construct on MSCs. The percentage of CD19-positive cells, a surrogate for successful transduction with iCasp9, remains constant for more than 2 weeks. To further address the stability of the construct, a population of iCasp9-positive cells purified by a fluorescence activated cell sorter (FACS) was maintained in culture: no significant difference in the percentage of CD19-positive cells was observed over six weeks (96.5±1.1% at baseline versus 97.4±0.8% after 43 days, P=0.46). The phenotype of the iCasp9-CD19-positive cells was otherwise substantially identical to that of untransduced cells, with virtually all cells positive for CD73, CD90 and CD105 and negative for hematopoietic markers, confirming that the genetic manipulation of MSCs did not modify their basic characteristics.

iCasp9-ΔCD19 Transduced MSCs Undergo Selective Apoptosis after Exposure to CID In Vitro

The proapoptotic gene product iCasp9 can activated by a small chemical inducer of dimerization (CID), AP20187, an analogue of tacrolimus that binds the FK506-binding domain present in the iCasp9 product. Non-transduced MSCs have a spontaneous rate of apoptosis in culture of approximately 18% (±7%) as do iCasp9-positive cells at baseline (15±6%, P=0.47). Addition of CID (50 nM) to MSC cultures after transduction with iCasp9-ΔCD19 results in the apoptotic death of more than 90% of iCasp9-positive cells within 24 hrs (93±1%, P<0.0001), while iCasp9-negative cells retain an apoptosis index similar to that of non-transduced controls (20±7%, P=0.99 and P=0.69 vs. non-transduced controls with or without CID respectively) (see FIGS. 17A and 70B). After transduction of MSCs with iCasp9, the chemical inducer of dimerization (CID) was added at 50 nM to cultures in complete medium. Apoptosis was evaluated 24 hours later by FACS analysis, after cell harvest and staining with annexin V-PE and 7-AAD. Ninety-three percent of the iCasp9-CD19-positive cells (iCasp pos/CID) became annexin positive versus only 19% of the negative population (iCasp neg/CID), a proportion comparable to non-transduced control MSC exposed to the same compound (Control/CID, 15%) and to iCasp9-CD19-positive cells unexposed to CID (iCasp pos/no CID, 13%), and similar to the baseline apoptotic rate of non-transduced MSCs (Control/no CID, 16%). Magnetic immunoselection of iCasp9-CD19-positive cells can be achieved to high degree of purity. More than 95% of the selected cells become apoptotic after exposure to CID.

Analysis of a highly purified iCasp9-positive population at later time points after a single exposure to CID shows that the small fraction of iCasp9-negative cells expands and that a population of iCasp9-positive cells remains, but that the latter can be killed by re-exposure to CID. Thus, no iCasp9-positive population resistant to further killing by CID was detected. A population of iCasp9-CD19-negative MSCs emerges as early as 24 hours after CID introduction. A population of iCasp9-CD19-negative MSCs is expected since achieving a population with 100% purity is unrealistic and because the MSCs are being cultured in conditions that favor their rapid expansion in vitro. A fraction of iCasp9-CD19-positive population persists, as predicted by the fact that killing is not 100% efficient (assuming, for example, 99% killing of a 99% pure population, the resulting population would have 49.7% iCasp9-positive and 50.3% iCasp9-negative cells). The surviving cells, however, can be killed at later time points by re-exposure to CID.

iCasp9-ΔCD19 Transduced MSCs Maintain the Differentiation Potential of Unmodified MSCs and their Progeny is Killed by Exposure to CID

To determine if the CID can selectively kill the differentiated progeny of iCasp9-positive MSCs, immunomagnetic selection for CD19 was used to increase the purity of the modified population (>90% after one round of selection. The iCasp9-positive cells thus selected were able to differentiate in vivo into all connective tissue lineages studied (see FIGS. 19A-19Q). Human MSCs were immunomagnetically selected for CD19 (thus iCasp9) expression, with a purity greater than 91%. After culture in specific differentiation media, iCasp9-positive cells were able to give rise to adipocytic (A, oil red and methylene azure), osteoblastic (B, alkaline phosphatase-BCIP/NBT and methylene blue) and chondroblastic lineages (C, alcian blue and nuclear red) lineages. These differentiated tissues are driven to apoptosis by exposure to 50 nM CID (D-N). Note numerous apoptotic bodies (arrows), cytoplasmic membrane blebbing (inset) and loss of cellular architecture (D and E); widespread TUNEL positivity in chondrocytic nodules (F-H), and adipogenic (I-K) and osteogenic (L-N) cultures, in contrast to that seen in untreated iCasp9-transduced controls (adipogenic condition shown, O-Q) (F, I, L, O, DAPI; G, J, M, P, TUNEL-FITC; H, K, N, Q, overlay).

After 24 hours of exposure to 50 nM of CID, microscopic evidence of apoptosis was observed with membrane blebbing, cell shrinkage and detachment, and presence of apoptotic bodies throughout the adipogenic and osteogenic cultures. A TUNEL assay showed widespread positivity in adipogenic and osteogenic cultures and the chondrocytic nodules (see FIGS. 19A-19Q), which increased over time. After culture in adipocytic differentiation media, iCasp9-positive cells gave rise to adipocytes. After exposure to 50 nM CID, progressive apoptosis was observed as evidenced by an increasing proportion of TUNEL-positive cells. After 24 hours, there was a significant decrease in cell density (from 584 cells/mm² to <14 cells/mm²), with almost all apoptotic cells having detached from the slides, precluding further reliable calculation of the proportion of apoptotic cells. Thus, iCasp9 remained functional even after MSC differentiation, and its activation results in the death of the differentiated progeny.

iCasp9-ΔCD19 Transduced MSCs Undergo Selective Apoptosis after In Vivo Exposure to CID

Although intravenously injected MSC already appear to have a short in vivo survival time, cells injected locally may survive longer and produce correspondingly more profound adverse effects. To assess the in vivo functionality of the iCasp9 suicide system in such a setting, SCID mice were subcutaneously injected with MSCs. MSCs were doubly transduced with the eGFP-FFLuc (previously presented) and iCasp9-ΔCD19 genes. MSCs were also singly transduced with eGFP-FFLuc. The eGFP-positive (and CD19-positive, where applicable) fractions were isolated by fluorescence activated cell sorting, with a purity >95%. Each animal was injected subcutaneously with iCasp9-positive and control MSCs (both eGFP-FFLuc-positive) in opposite flanks. Localization of the MSCs was evaluated using the Xenogen-IVIS Imaging System. In another set of experiments, a 1:1 mixture of singly and doubly transduced MSCs was injected subcutaneously in the right flank and the mice received CID as above. The subcutaneous pellet of MSCs was harvested at different time points, genomic DNA was isolated and qPCR was used to determine copy numbers of the eGFP-FFLuc and iCasp9-ΔCD19 genes. Under these conditions, the ratio of the iCasp9 to eGFP gene copy numbers is proportional to the fraction of iCasp9-positive cells among total human cells (see Methods above for details). The ratios were normalized so that time zero corresponds to 100% of iCasp9-positive cells. Serial examination of animals after subcutaneous inoculation of MSCs (prior to CID injection) shows evidence of spontaneous apoptosis in both cell populations (as demonstrated by a fall in the overall luminescence signal to ˜20% of the baseline). This has been previously observed after systemic and local delivery of MSCs in xenogeneic models.

The luminescence data showed a substantial loss of human MSCs over the first 96 h after local delivery of MSCs, even before administration of CID, with only approximately 20% cells surviving after one week. From that time point onward, however, there were significant differences between the survival of icasp9-positive MSCs with and without dimerizer drug. Seven days after MSC implantation, animals were given two injections of 50 μg of CID, 24 hours apart. MSCs transduced with iCasp9 were quickly killed by the drug, as demonstrated by the disappearance of their luminescence signal. Cells negative for iCasp9 were not affected by the drug. Animals not injected with the drug showed persistence of signal in both populations up to a month after MSC implantation. To further quantify cell killing, qPCR assays were developed to measure copy numbers of the eGFP-FFLuc and iCasp9-ΔCD19 genes. Mice were injected subcutaneously with a 1:1 mixture of doubly and singly transduced MSCs and administered CID as above, one week after MSC implantation. MSCs explants were collected at several time points, genomic DNA isolated from the samples and qPCR assays performed on substantially identical amounts of DNA. Under these conditions (see Methods), at any time point, the ratio of iCasp9-ΔCD19 to eGFP-FFLuc copy numbers is proportional to the fraction of viable iCasp9-positive cells. Progressive killing of iCasp9-positive cells was observed (>99%) so that the proportion of surviving iCasp9-positive cells was reduced to 0.7% of the original population after one week. Therefore, MSCs transduced with iCasp9 can be selectively killed in vivo after exposure to CID, but otherwise persist.

### Discussion

The feasibility of engineering human MSCs to express a safety mechanism using an inducible suicide protein is demonstrated herein. The date presented herein show that MSC can be readily transduced with the suicide gene iCasp9 coupled to the selectable surface maker CD19. Expression of the co-transduced genes is stable both in MSCs and their differentiated progeny, and does not evidently alter their phenotype or potential for differentiation. These transduced cells can be killed in vitro and in vivo when exposed to the appropriate small molecule chemical inducer of dimerization that binds to the iCasp9.

For a cell based therapy to be successful, transplanted cells must survive the period between their harvest and their ultimate in vivo clinical application. Additionally, a safe cell based therapy also should include the ability to control the unwanted growth and activity of successfully transplanted cells. Although MSCs have been administered to many patients without notable side effects, recent reports indicate additional protections, such as the safety switch presented herein, may offer additional methods of control over cell based therapies as the potential of transplanted MSC to be genetically and epigenetically modified to enhance their functionality, and to differentiate into lineages including bone and cartilage is further investigated and exploited. Subjects receiving MSCs that have been genetically modified to release biologically active proteins might particularly benefit from the added safety provided by a suicide gene.

The suicide system presented herein offers several potential advantages over other known suicide systems. Strategies involving nucleoside analogues, such as those combining Herpes Simplex Virus thymidine kinase (HSV-tk) with gancyclovir (GCV) and bacterial or yeast cytosine deaminase (CD) with 5-fluoro-cytosine (5-FC), are cell-cycle dependent and are unlikely to be effective in the post-mitotic tissues that may be formed during the application of MSCs to regenerative medicine. Moreover, even in proliferating tissues the mitotic fraction does not comprise all cells, and a significant portion of the graft may survive and remain dysfunctional. In some instance, the prodrugs required for suicide may themselves have therapeutic uses that are therefore excluded (e.g., GCV), or may be toxic (e.g., 5-FC), either as a result of their metabolism by non-target organs (e.g., many cytochrome P450 substrates), or due to diffusion to neighboring tissues after activation by target cells (e.g., CB1954, a substrate for bacterial nitroreductase).

In contrast, the small molecule chemical inducers of dimerization presented herein have shown no evidence of toxicities even at doses ten fold higher than those required to activate the iCasp9. Additionally, nonhuman enzymatic systems, such as HSV-tk and DC, carry a high risk of destructive immune responses against transduced cells. Both the iCasp9 suicide gene and the selection marker CD19, are of human origin, and thus should be less likely to induce unwanted immune responses. Although linkage of expression of the selectable marker to the suicide gene by a 2A-like cleavable peptide of nonhuman origin could pose problems, the 2A-like linker is 20 amino acids long, and is likely less immunogenic than a nonhuman protein. Finally, the effectiveness of suicide gene activation in iCasp9-positive cells compares favorably to killing of cells expressing other suicide systems, with 90% or more of iCasp9-modified T cells eliminated after a single dose of dimerizer, a level that is likely to be clinically efficacious.

The iCasp9 system presented herein also may avoid additional limitations seen with other cell based and/or suicide switch based therapies. Loss of expression due to silencing of the transduced construct is frequently observed after retroviral transduction of mammalian cells. The expression constructs presented herein showed no evidence of such an effect. No decrease in expression or induced death was evident, even after one month in culture.

Another potential problem sometimes observed in other cell based and/or suicide switch based therapies, is the development of resistance in cells that have upregulated anti-apoptotic genes. This effect has been observed in other suicide systems involving different elements of the programmed cell death pathways such as Fas. iCasp9 was chosen as the suicide gene for the expression constructs presented herein because it was less likely to have this limitation. Compared to other members of the apoptotic cascade, activation of Caspase-9 occurs late in the apoptotic pathway and therefore should bypass the effects of many if not all anti-apoptotic regulators, such as c-FLIP and bcl-2 family members.

A potential limitation specific to the system presented herein may be spontaneous dimerization of iCasp9, which in turn could cause unwanted cell death and poor persistence. This effect has been observed in certain other inducible systems that utilize Fas. The observation of low spontaneous death rate in transduced cells and long term persistence of transgenic cells in vivo indicate this possibility is not a significant consideration when using iCasp9 based expression constructs.

Integration events deriving from retroviral transduction of MSCs may potentially drive deleterious mutagenesis, especially when there are multiple insertions of the retroviral vector, causing unwanted copy number effects and/or other undesirable effects. These unwanted effects could offset the benefit of a retrovirally transduced suicide system. These effects often can be minimized using clinical grade retroviral supernatant obtained from stable producer cell lines and similar culture conditions to transduce T lymphocytes. The T cells transduced and evaluated herein contain in the range of about 1 to 3 integrants (the supernatant containing in the range of about 1×10⁶ viral particles/mL). The substitution of lentiviral for retroviral vectors could further reduce the risk of genotoxicity, especially in cells with high self-renewal and differentiation potential.

While a small proportion of iCasp9-positive MSCs persists after a single exposure to CID, these surviving cells can subsequently be killed following re-exposure to CID. In vivo, there is >99% depletion with two doses, but it is likely that repeated doses of CID will be needed for maximal depletion in the clinical setting. Additional non-limiting methods of providing extra safety when using an inducible suicide switch system include additional rounds of cell sorting to further increase the purity of the cell populations administered and the use of more than one suicide gene system to enhance the efficiency of killing.

The CD19 molecule, which is physiologically expressed by B lymphocytes, was chosen as the selectable marker for transduced cells, because of its potential advantages over other available selection systems, such as neomycin phosphotransferase (neo) and truncated low affinity nerve growth factor receptor (ΔLNGFR). “neo” encodes a potentially immunogenic foreign protein and requires a 7-day culture in selection medium, increasing the complexity of the system and potentially damaging the selected cells. ΔLNGFR expression should allow for isolation strategies similar to other surface markers, but these are not widely available for clinical use and a lingering concern remains about the oncogenic potential of ΔLNGFR. In contrast, magnetic selection of iCasp9-positive cells by CD19 expression using a clinical grade device is readily available and has shown no notable effects on subsequent cell growth or differentiation.

The procedure used for preparation and administration of mesenchymal stromal cells comprising the Caspase-9 safety switch may also be used for the preparation of embryonic stem cells and inducible pluripotent stem cells. Thus for the procedures outlined in the present example, either embryonic stem cells or inducible pluripotent stem cells may be substituted for the mesenchymal stromal cells provided in the example. In these cells, retroviral and lentiviral vectors may be used, with, for example, CMV promoters, or the ronin promoter.

### Example 6: Modified Caspase-9 Polypeptides with Lower Basal Activity and Minimal Loss of Ligand IC₅₀

Basal signaling, signaling in the absence of agonist or activating agent, is prevalent in a multitude of biomolecules. For example, it has been observed in more than 60 wild-type G protein coupled receptors (GPCRs) from multiple subfamilies [1], kinases, such as ERK and abl [2], surface immunoglobulins [3], and proteases. Basal signaling has been hypothesized to contribute to a vast variety of biological events, from maintenance of embryonic stem cell pluripotency, B cell development and differentiation [4-6], T cell differentiation [2, 7], thymocyte development [8], endocytosis and drug tolerance [9], autoimmunity [10], to plant growth and development [11]. While its biological significance is not always fully understood or apparent, defective basal signaling can lead to serious consequences. Defective basal Gₛ protein signaling has led to diseases, such as retinitis pigmentosa, color blindness, nephrogenic diabetes insipidus, familial ACTH resistance, and familial hypocalciuric hypercalcemia [12, 13].

Even though homo-dimerization of wild-type initiator Caspase-9 is energetically unfavorable, making them mostly monomers in solution [14-16], the low-level inherent basal activity of unprocessed Caspase-9 [15, 17] is enhanced in the presence of the Apaf-1-based “apoptosome”, its natural allosteric regulator [6]. Moreover, supra-physiological expression levels and/or co-localization could lead to proximity-driven dimerization, further enhancing basal activation.

In the chimeric unmodified Caspase-9 polypeptide, innate Caspase-9 basal activity was significantly diminished by removal of the CAspase-Recruitment pro-Domain (CARD) [18], replacing it with the cognate high affinity AP1903-binding domain, FKBP12-F36V. Its usefulness as a pro-apoptotic “safety switch” for cell therapy has been well demonstrated in multiple studies [18-20]. While its high specific and low basal activity has made it a powerful tool in cell therapy, in contrast to G protein coupled receptors, there are currently no “inverse agonists” [21] to eliminate basal signaling, which may be desirable for manufacturing, and in some applications. Preparation of Master Cell Banks has proven challenging due to high amplification of the low-level basal activity of the chimeric polypeptide. In addition, some cells are more sensitive than others to low-level basal activity of Caspase-9, leading to unintended apoptosis of transduced cells [18].

To modify the basal activity of the chimeric Caspase-9 polypeptide, “rational design”-based methods were used to engineer 75i Casp9 mutants based on residues known to play crucial roles in homo-dimerization, XIAP-mediated inhibition, or phosphorylation (Table below) rather than “directed evolution” [22] that use multiple cycles of screening as selective pressure on randomly generated mutants. Dimerization-driven activation of Caspase-9 has been considered a dominant model of initiator Caspase activation [15, 23, 24]. To reduce spontaneous dimerization, site-directed mutagenesis was conducted of residues crucial for homo-dimerization and thus basal Caspase-9 signaling. Replacement of five key residues in the β6 strand (G402-C-F-N-F406 (SEQ ID NO: 297)), the key dimerization interface of Caspase-9, with those of constitutively dimeric effector Caspase-3 (C264-I-V-S-M268 (SEQ ID NO: 298)) converted it to a constitutively dimeric protein unresponsive to Apaf-1 activation without significant structural rearrangements [25]. To modify spontaneous homo-dimerization, systemic mutagenesis of the five residues was made, based on amino acid chemistry, and on corresponding residues of initiator Caspases-2, -8, -9, and -10 that exist predominately as a monomer in solution [14, 15]. After making and testing twenty-eight iCasp9 mutants by a secreted alkaline phosphatase (SEAP)-based surrogate killing assay (Table, below), the N405Q mutation was found to lower basal signaling with a moderate (<10-fold) cost of higher IC₅₀ to AP1903.

Since proteolysis, typically required for Caspase activation, is not absolutely required for Caspase-9 activation [26], the thermodynamic “hurdle” was increased to inhibit auto-proteolysis. In addition, since XIAP-mediated Caspase-9 binding traps Caspase-9 in a monomeric state to attenuate its catalytic and basal activity [14], there was an effort to strengthen the interaction between XIAP and Caspase-9 by mutagenizing the tetrapeptide critical for interaction with XIAP (A316-T-P-F319 (SEQ ID NO: 299), D330-A-I-S-5334 (SEQ ID NO: 301)). From 17 of these iCasp-9 mutants, it was determined that the D330A mutation lowered basal signaling with a minimum (<5-fold) AP1903 IC₅₀ cost.

The third approach was based on previously reported findings that Caspase-9 is inhibited by kinases upon phosphorylation of S144 by PKC-ζ [27], S183 by protein kinase A [28], S196 by Akt1 [29], and activated upon phosphorylation of Y153 by c-abl [30]. These “brakes” might improve the IC₅₀, or substitutions with phosphorylation mimic (“phosphomimetic”) residues could augment these “brakes” to lower basal activity. However, none of the 15 single residue mutants based on these residues successfully lowered the IC₅₀ to AP1903.

Methods such as those discussed, for example, in Examples 1-5, and throughout the present application may be applied, with appropriate modifications, if necessary to the chimeric modified Caspase-9 polypeptides, as well as to various therapeutic cells.

### Example 7: Materials and Methods

### PCR Site-Directed Mutagenesis of Caspase-9:

To modify basal signaling of Caspase-9, PCR-based site directed mutagenesis [31] was done with mutation-containing oligos and Kapa (Kapa Biosystems, Woburn, Mass.). After 18 cycles of amplification, parental plasmid was removed with methylation-dependent DpnI restriction enzyme that leaves the PCR products intact. 2 μl of resulting reaction was used to chemically transform XL1-blue or DH5a. Positive mutants were subsequently identified via sequencing (SeqWright, Houston, Tex.).

### Cell Line Maintenance and Transfection:

Early passage HEK293T/16 cells (ATCC, Manassas, Va.) were maintained in IMDM, GlutaMAX™ (Life Technologies, Carlsbad, Calif.) supplemented with 10% FBS, 100 U/mL penicillin, and 100 U/mL streptomycin until transfection in a humidified, 37° C., 5% CO₂/95% air atmosphere. Cells in logarithmic-phase growth were transiently transfected with 800 ng to 2 μg of expression plasmid encoding iCasp9 mutants and 500 ng of an expression plasmid encoding SRα promoter driven SEAP per million cells in 15-mL conical tubes. Catalytically inactive Caspase-9 (C285A) (without the FKBP domain) or “empty” expression plasmid (“pSH1-null”) were used to keep the total plasmid levels constant between transfections. GeneJammer® Transfection Reagent at a ratio of 3 μl per ug of plasmid DNA was used to transiently transfect HEK293T/16 cells in the absence of antibiotics. 100 μl or 2 mL of the transfection mixture was added to each well in 96-well or 6-well plate, respectively. For SEAP assays, log dilutions of AP1903 were added after a minimum 3-hour incubation post-transfection. For western blots, cells were incubated for 20 minutes with AP1903 (10 nM) before harvesting.

### Secreted Alkaline Phosphatase (SEAP) Assay:

Twenty-four to forty-eight hours after AP1903 treatment, ˜100 μl of supernatants were harvested into a 96-well plate and assayed for SEAP activity as discussed [19, 32]. Briefly, after 65° C. heat denaturation for 45 minutes to reduce background caused by endogenous (and serum-derived) alkaline phosphatases that are sensitive to heat, 5 μl of supernatants was added to 95 μl of PBS and added to 100 μl of substrate buffer, containing 1 μl of 100 mM 4-methylumbelliferyl phosphate (4-MUP; Sigma, St. Louis, Mo.) re-suspended in 2 M diethanolamine. Hydrolysis of 4-MUP by SEAP produces a fluorescent substrate with excitation/emission (355/460 nm), which can be easily measured. Assays were performed in black opaque 96-well plates to minimize fluorescence leakage between wells. To examine both basal signaling and AP1903 induced activity, 106 early-passage HEK293T/16 cells were co-transfected with various amount of wild type Caspase and 500 ng of an expression plasmid that uses an SRα promoter to drive SEAP, a marker for cell viability. Following manufacturer's suggestions, 1 mL of IMDM+10% FBS without antibiotics was added to each mixture. 1000-μI of the mixture was seeded onto each well of a 96-well plate. 100-μI of AP1903 was added at least three hours post-transfection. After addition of AP1903 for at least 24 hours, 100-μl of supernatant was transferred to a 96-well plate and heat denatured at 68° C. for 30 minutes to inactivate endogenous alkaline phosphatases. For the assay, 4-methylumbelliferyl phosphate substrate was hydrolyzed by SEAP to 4-methylumbelliferon, a metabolite that can be excited with 364 nm and detected with an emission filter of 448 nm. Since SEAP is used as a marker for cell viability, reduced SEAP reading corresponds with increased iCaspase-9 activities. Thus, a higher SEAP reading in the absence of AP1903 would indicate lower basal activity. Desired caspase mutants would have diminished basal signaling with increased sensitivity (i.e., lower IC₅₀) to AP1903. The goal of the study is to reduce basal signaling without significantly impairing IC₅₀.

### Western Blot Analysis:

HEK293T/16 cells transiently transfected with 2 μg of plasmid for 48-72 hours were treated with AP1903 for 7.5 to 20 minutes (as indicated) at 37° C. and subsequently lysed in 500 μl of RIPA buffer (0.01 M Tris-HCl, pH 8.0/140 mM NaCl/1% Triton X-100/1 mM phenylmethylsulfonyl fluoride/1% sodium deoxycholate/0.1% SDS) with Halt™ Protease Inhibitor Cocktail. The lysates were collected and lysed on ice for 30 min. After pelleting cell debris, protein concentrations from overlying supernatants were measured in 96-well plates with BCA™

Protein Assay as recommended by the manufacturer. 30 μg of proteins were boiled in Laemmli sample buffer (Bio-Rad, Hercules, Calif.) with 2.5% 2-mercaptoethanol for 5 min at 95° C. before being separated by Criterion TGX 10% Tris/glycine protein gel. Membranes were probed with 1/1000 rabbit anti-human Caspase-9 polyclonal antibody followed by 1/10,000 HRP-conjugated goat anti-rabbit IgG F(ab′)2 secondary antibody (Bio-Rad). Protein bands were detected using Supersignal West Femto chemiluminescent substrate. To ensure equivalent sample loading, blots were stripped at 65° C. for 1 hour with Restore PLUS Western Blot Stripping Buffer before labeling with 1/10,000 rabbit anti-actin polyclonal antibody. Unless otherwise stated, all the reagents were purchased from Thermo Scientific.

Methods and constructs discussed in Examples 1-5, and throughout the present specification may also be used to assay and use the modified Caspase-9 polypeptides.

### Example 8: Evaluation and Activity of Chimeric Modified Caspase-9 Polypeptides

### Comparison of Basal Activity and AP1903 Induced Activity:

To examine both basal activity and AP1903 induced activity of the chimeric modified Caspase-9 polypeptides, SEAP activities of HEK293T/16 cells co-transfected with SEAP and different amounts of iCasp9 mutants were examined. iCasp9 D330A, N405Q, and D330A-N405Q showed significantly less basal activity than unmodified iCasp9 for cells transfected with either 1 μg iCasp9 per million cells (relative SEAP activity Units of 148928, 179081, 205772 vs. 114518) or 2 μg iCasp9 per million cells (136863, 175529, 174366 vs. 98889). The basal signaling of all three chimeric modified Caspase-9 polypeptides when transfected at 2 μg per million cells was significantly higher (p value<0.05). iCasp9 D330A, N405Q, and D330A-N405Q also showed increased estimated IC₅₀s for AP1903, but they are all still less than 6 μM (based on the SEAP assay), compared to 1 μM for WT, making them potentially useful apoptosis switches.

### Evaluation of Protein Expression Levels and Proteolysis:

To exclude the possibility that the observed reduction in basal activity of the chimeric modified Caspase-9 polypeptides was attributable to decreased protein stability or variation in transfection efficiency, and to examine auto-proteolysis of iCasp9, the protein expression levels of Caspase-9 variants in transfected HEK293T/16 cells was assayed. Protein levels of chimeric unmodified Caspase-9 polypeptide, iCasp9 D330A, and iCasp9 D330A-N405Q all showed similar protein levels under the transfection conditions used in this study. In contrast, the iCasp9 N405Q band appeared darker than the others, particularly when 2 μg of expression plasmids was used. Auto-proteolysis was not easily detectable at the transfection conditions used, likely because only viable cells were collected. Anti-actin protein reblotting confirmed that comparable lysate amounts were loaded into each lane. These results support the observed lower basal signaling in the iCasp9 D330A, N405Q, and D330A-N405Q mutants, observed by SEAP assays.

### Discussion

Based on the SEAP screening assay, these three chimeric modified Caspase-9 polypeptides showed higher AP1903-independent SEAP activity, compared to iCasp9 WT transfectants, and hence lower basal signaling. However, the double mutation (D330-N405Q) failed to further decrease either basal activity or IC₅₀ (0.05 nM) vs. the single amino acid mutants. The differences observed did not appear to be due to protein instability or differential amount of plasmids used during transfection.

### Example 9: Evaluation and Activity of Chimeric Modified Caspase-9 Polypeptides

Inducible Caspase-9 provides for rapid, cell-cycle-independent, cell autonomous killing in an AP1903-dependent fashion. Improving the characteristics of this inducible Caspase-9 polypeptide would allow for even broader applicability. It is desirable to decrease the protein's ligand-independent cytotoxicity, and increase its killing at low levels of expression. Although ligand-independent cytotoxicity is not a concern at relatively low levels of expression, it can have a material impact where levels of expression can reach one or more orders of magnitude higher than in primary target cells, such as during vector production. Also, cells can be differentially sensitive to low levels of caspase expression due to the level of apoptosis inhibitors, like XIAP and Bcl-2, which cells express. Therefore, to re-engineer the caspase polypeptide to have a lower basal activity and possibly higher sensitivity to AP1903 ligand, four mutagenesis strategies were devised.

Dimerization Domain: Although Caspase-9 is a monomer in solution at physiological levels, at high levels of expression, such as occurs in the pro-apoptotic, Apaf-driven “apoptosome”, Caspase-9 can dimerize, leading to auto-proteolysis at D315 and a large increase in catalytic activity. Since C285 is part of the active site, mutation C285A is catalytically inactive and is used as a negative control construct. Dimerization involves very close interaction of five residues in particular, namely G402, C403, F404, N405, and F406. For each residue, a variety of amino acid substitutions, representing different classes of amino acids (e.g., hydrophobic, polar, etc.) were constructed. Interestingly, all mutants at G402 (i.e., G402A, G4021, G402Q, G402Y) and C403P led to a catalytically inactive caspase polypeptide. Additional C403 mutations (i.e., C403A, C4035, and C403T) were similar to the wild type caspase and were not pursued further. Mutations at F404 all lowered basal activity, but also reflected reduced sensitivity to IC₅₀, from ˜1 log to unmeasurable. In order of efficacy, they are: F404Y>F404T, F404W>>F404A, F4045. Mutations at N405 either had no effect, as with N405A, increased basal activity, as in N405T, or lowered basal activity concomitant with either a small (˜5-fold) or larger deleterious effect on IC₅₀, as with N405Q and N405F, respectively. Finally, like F404, mutations at F406 all lowered basal activity, and reflected reduced sensitivity to IC₅₀, from ˜1 log to unmeasurable. In order of efficacy, they are: F406A F406W, F406Y>F406T>>F406L.

Some polypeptides were constructed and tested that had compound mutations within the dimerization domain, but substituting the analogous 5 residues from other caspases, known to be monomers (e.g., Caspase-2, -8, -10) or dimers (e.g., Caspase-3) in solution. Caspase-9 polypeptides, containing the 5-residue change from Caspase-2, -3, and -8, along with an AAAAA (SEQ ID NO: 302) alanine substitution were all catalytically inactive, while the equivalent residues from Caspase-10 (ISAQT (SEQ ID NO: 303)), led to reduced basal activity but higher IC₅₀.

Overall, based on the combination of consistently lower basal activity, combined with only a mild effect on IC₅₀, N405Q was selected for further experiments. To improve on efficacy, a codon-optimized version of the modified Caspase-9 polypeptide, having the N405Q substitution, called N405Qco, was tested. This polypeptide appeared marginally more sensitive to AP1903 than the wild type N405Q-substituted Caspase-9 polypeptide.

Cleavage site mutants: Following aggregation of Caspase-9 within the apoptosome or via AP1903-enforced homodimerization, auto-proteolysis at D315 occurs. This creates a new amino-terminus at A316, at least transiently. Interestingly, the newly revealed tetra-peptide, ³¹⁶ATPF³¹⁹ (SEQ ID NO: 299), binds to the Caspase-9 inhibitor, XIAP, which competes for dimerization with Caspase-9 itself at the dimerization motif, GCFNF (SEQ ID NO: 297), discussed above. Therefore, the initial outcome of D315 cleavage is XIAP binding, attenuating further Caspase-9 activation. However, a second caspase cleavage site exists at D330, which is the target of downstream effector caspase, caspase-3. As the pro-apoptotic pressure builds, D330 becomes increasingly cleaved, releasing the XIAP-binding small peptide within residue 316 to 330, and hence, removing this mitigating Caspase-9 inhibitor. A D330A mutant was constructed, which lowered basal activity, but not as low as in N405Q. By SEAP assay at high copy number, it also revealed a slight increase in IC₅₀, but at low copy number in primary T cells, there was actually a slight increase in IC₅₀ with improved killing of target cells. Mutation at auto-proteolysis site, D315, also reduced basal activity, but this led to a large increase in IC₅₀, likely as D330 cleavage was then necessary for caspase activation. A double mutation at D315A and D330A, led to an inactive “locked” Caspase-9 that could not be processed properly.

Other D330 mutants were created, including D330E, D330G, D330N, D330S, and D330V. Mutation at D327 also prevented cleavage at D330, as the consensus Caspase-3 cleavage site is DxxD, but several D327 mutations (i.e., D327G, D327K, and D327R) along with F326K, Q328K, Q328R, L329K, L329G, and A331K, unlike D330 mutations, did not lower basal activity and were not pursued further.

XIAP-binding mutants: As discussed above, autoproteolysis at D315 reveals an XIAP-binding tetrapeptide, ³¹⁶ATPF³¹⁹ (SEQ ID NO: 299), which “lures” XIAP into the Caspase-9 complex. Substitution of ATPF (SEQ ID NO: 299) with the analogous XIAP-binding tetrapeptide, AVPI (SEQ ID NO: 304), from mitochondria-derived anti-XIAP inhibitor, SMAC/DIABLO, might bind more tightly to XIAP and lower basal activity. However, this 4-residue substitution had no effect. Other substitutions within the ATPF motif (SEQ ID NO: 299) ranged from no effect, (i.e., T317C, P318A, F319A) to lower basal activity with either a very mild (i.e., T317S, mild (i.e., T317A) to large (i.e., A316G, F319W) increase in IC₅₀. Overall, the effects of changing the XIAP-binding tetrapeptide were mild; nonetheless, T317S was selected for testing in double mutations (discussed below), since the effects on IC₅₀ were the most mild of the group.

Phosphorylation mutants: A small number of Caspase-9 residues were reported to be the targets of either inhibitory (e.g., S144, S183, S195, S196, S307, T317) or activating (i.e., Y153) phosphorylations. Therefore, mutations that either mimic the phosphorylation (“phosphomimetics”) by substitution with an acidic residue (e.g., Asp) or eliminate phosphorylation were tested. In general, most mutations, regardless of whether a phosphomimetic or not was tried, lowered basal activity. Among the mutants with lower basal activity, mutations at S144 (i.e., S144A and S144D) and 51496D had no discernable effect on IC₅₀, mutants S183A, S195A, and S196A increased the IC₅₀ mildly, and mutants Y153A, Y153A, and S307A had a big deleterious effect on IC₅₀. Due to the combination of lower basal activity and minimal, if any effect on IC₅₀, S144A was chosen for double mutations (discussed below).

Double mutants: In order to combine the slightly improved efficacy of D330A variant with possible residues that could further lower basal activity, numerous D330A double mutants were constructed and tested. Typically, they maintained lower basal activity with only a slight increase in IC₅₀, including 2nd mutations at N405Q, S144A, S144D, S183A, and S196A. Double mutant D330A-N405T had higher basal activity and double mutants at D330A with Y153A, Y153F, and T317E were catalytically inactive. A series of double mutants with low basal activity N405Q, intended to improve efficacy or decrease the IC₅₀ was tested. These all appeared similar to N405Q in terms of low basal activity and slightly increased IC₅₀ relative to iC9-1.0, and included N405Q with S144A, S144D, S196D, and T317S.

SEAP assays were conducted to study the basal activity and CID sensitivity of some of the dimerization domain mutants. N405Q was the most AP1903-sensitive of the mutants tested with lower basal activity than the WT Caspase-9, as determined by a shift upwards of AP1903-independent signaling. F406T was the least CID-sensitive from this group.

The dimer-independent SEAP activity of mutant caspase polypeptides D330A and N405Q was assayed, along with double mutant D330A-N405Q. The results of multiple transfections (N=7 to 13) found that N405Q has lower basal activity than D330A and the double mutant is intermediate.

Obtaining the average (+stdev, n=5) IC₅₀ of mutant caspase polypeptides D330A and N405Q, along with double mutant D330A-N405Q shows that D330A is somewhat more sensitive to AP1903 than N405Q mutants but about 2-fold less sensitive than WT Caspase-9 in a transient transfection assay.

SEAP assays were conducted using wild type (WT) Caspase-9, N405Q, inactive C285A, and several T317 mutants within the XIAP-binding domain. The results show that T317S and T317A can reduce basal activity without a large shift in the IC₅₀ to APf1903. Therefore, T317S was chosen to make double mutants with N405Q.

IC₅₀s from the SEAP assays above showed that T317A and T317S have similar IC₅₀s to wild type Caspase-9 polypeptide despite having lower basal activity.

The dimer-independent SEAP activity from several D330 mutants showed that all members of this class tested, including D330A, D330E, D330N, D330V, D330G, and D330S, have less basal activity than wild type Caspase-9. Basal and AP1903-induced activation of D330A variants was assayed. SEAP assay of transiently transfected HEK293/16 cells with 1 or 2 ug of mutant caspase polypeptides and 0.5 ug of pSH1-kSEAP per million HEK293 cells, 72 hours post-transfection. Normalized data based on 2 ug of each expression plasmid (including WT) were mixed with normalized data from 1 ug-based transfections. iCasp9-D330A, -D330E, and -D330S showed statistically lower basal signaling than wildtype Caspase-9.

The result of a western blot shoed that the D330 mutations block cleavage at D330, leading to a slightly largely (slower migrating) small band (<20 kDa marker). Other blots show that D327 mutation also blocks cleavage.

The mean fluorescence intensities of multiple clones of PG13 transduced 5× with retroviruses encoding the indicated Caspase-9 polypeptides was measured. Lower basal activity typically translates to higher levels of expression of the Caspase-9 gene along with the genetically linked reporter, CD19. The results show that on the average, clones expressing the N405Q mutant express higher levels of CD19, reflecting the lower basal activity of N405Q over D330 mutants or WT Caspase-9. The effects of various caspase mutations on viral titers derived from PG13 packaging cells cross-transduced with VSV-G envelope-based retroviral supernatants was assayed. To examine the effect of iC9-derived basal signaling on retrovirus master cell line production, retrovirus packaging cell line, PG13, was cross-transduced five times with VSV-G-based retroviral supernatants in the presence of 4 μg/ml transfection-enhancer, polybrene. iC9-transduced PG13 cells were subsequently stained with PE-conjugated anti-human CD19 antibody, as an indication of transduction. iC9-D330A, -D330E, and -N405Q-transduced PG13 cells showed enhanced CD19 mean fluorescence intensity (MFI), indicating higher retroviral copy numbers, implying lower basal activity. To more directly examine the viral titer of the PG13 transductants, HT1080 cells were treated with viral supernatant and 8 ug/ml polybrene. The enhanced CD19 MFIs of iCasp9-D330A, -N405Q, and -D330E transductants vs WT iCasp9 in PG13 cells are positively correlated with higher viral titers, as observed in HT1080 cells. Due to the initially low viral titers (approximately 1E5 transduction units (TU)/ml), no differences in viral titers were observed in the absence of HAT treatment to increase virus yields. Upon HAT media treatment, PG13 cells transduced with iC9-D330A, -N405Q, or -D330E demonstrated higher viral titers. Viral titer (transducing units) is calculated with the formula: Viral titer=(# cells on the day of transduction)*(% CD19⁺)/Volume of supernatant (ml). In order to further investigate the effect of iC9 mutants with lower basal activity, individual clones (colonies) of iC9-transduced PG13 cells were selected and expanded. iC9-N405Q clones with higher CD19 MFIs than the other cohorts were observed.

The effects of various caspase polypeptides at mostly single copy in primary T cells was assayed. This may reflect more accurately how these suicide genes will be used therapeutically. Surprisingly, the data show that the D330A mutant is actually more sensitive to AP1903 at low titers and kills at least as well as WT Caspase-9 when tested in a 24-hour assay. The N405Q mutant is less sensitive to AP1903 and cannot kill target cells as efficiently within 24 hours.

Results of transducing 6 independent T cell samples from separate healthy donors showed that the D330A mutant (mut) is more sensitive to AP1903 than the wild type Caspase-9 polypeptide.

FIG. 57 shows the average IC₅₀, range and standard deviation from the 6 healthy donors shown in FIG. 56. This data shows that the improvement is statistically significant. The iCasp9-D330A mutant demonstrated improved AP1903-dependent cytotoxicity in transduced T cells. Primary T cells from healthy donors (n=6) were transduced with retrovirus encoding mutant or wild-type iCasp9 or iCasp9-D330A, and the ΔCD19 cell surface marker. Following transduction, iCasp9-transduced T cells were purified using CD19-microbeads and a magnetic column. T cells were then exposed to AP1903 (0-100 nM) and measured for CD3⁺CD19⁺ T cells by flow cytometry after 24 hours. The IC₅₀ of iCasp9-D330A was significantly lower (p=0.002) than wild-type iCasp9.

Results of several D330 mutants, revealed that all six D330 mutants tested (D330A, E, N, V, G, and S) are more sensitive to AP1903 than wild type Caspase-9 polypeptide.

The N405Q mutant along with other dimerization domain mutants, including N404Y and N406Y, can kill target T cells indistinguishable from wild type Caspase-9 polypeptide or D330A within 10 days. Cells that received AP1903 at Day 0 received a second dose of AP1903 at day 4. This data supports the use of reduced sensitivity Caspase-9 mutants, like N405Q as part of a regulated efficacy switch.

The results of codon optimization of N405Q caspase polypeptide, called “N405Qco”, revealed that codon optimization, likely leading to an increase in expression only has a very subtle effect on inducible caspase function. This likely reflects the use of common codons in the original Caspase-9 gene.

The Caspase-9 polypeptide has a dose-response curve in vivo, which could be used to eliminate a variable fraction of T cells expressing the Caspase-9 polypeptide. The data also shows that a dose of 0.5 mg/kg AP1903 is sufficient to eliminate most modified T cells in vivo.

AP1903 dose-dependent elimination in vivo of T cells transduced with D330E iCasp9 was assayed. T cells were transduced with SFG-iCasp9-D330E-2A-ΔCD19 retrovirus and injected i.v. into immune deficient mice (NSG). After 24 hours, mice were injected i.p. with AP1903 (0-5 mg/kg). After an additional 24 hours, mice were sacrificed and lymphocytes from the spleen (A) were isolated and analyzed by flow cytometry for the frequency of human CD3⁺CD19⁺ T cells. This shows that iCasp9-D330E demonstrates a similar in vivo cytotoxicity profile in response to AP1903 as wild-type iCasp9.

Conclusions: As discussed, from this analysis of 78 mutants so far, out of the single mutant mutations, the D330 mutations combine somewhat improved efficacy with slightly reduced basal activity. N405Q mutants are also attractive since they have very low basal activity with only slightly decreased efficacy, reflected by a 4-5-fold increase in IC₅₀. Experiments in primary T cells have shown that N405Q mutants can effectively kill target cells, but with somewhat slower kinetics than D330 mutants, making this potentially very useful for a graduated suicide switch that kills partially after an initial dose of AP1903, and up to full killing can be achieved upon a second dose of AP1903.

The following table provides a summary of basal activity and IC₅₀ for various chimeric modified Caspase-9 polypeptides prepared and assayed according to the methods discussed herein. The results are based on a minimum of two independent SEAP assays, except for a subset (i.e., A316G, T317E, F326K, D327G, D327K, D327R, Q328K, Q328R, L329G, L329K, A331K, S196A, S196D, and the following double mutants: D330A with S144A, S144D, or S183A; and N405Q with S144A, S144D, S196D, or T317S) that were tested once. Four multi-pronged approaches were taken to generate the tested chimeric modified Caspase-9 polypeptides. “Dead” modified Caspase-9 polypeptides were no longer responsive to AP1903. Double mutants are indicated by a hyphen, for example, D330A-N405Q denotes a modified Caspase-9 polypeptide having a substitution at position 330 and a substitution at position 405.

TABLE 5
Caspase Mutant Classes

| | | Cleavage sites | | | |
|---|---|---|---|---|---|
| | Homodimerization | & XIAP | | Double | |
| Basal Activity | domain | Interaction | Phosphorylation | mutants, Misc. | Total mutants |
| Decreased | | | S144A | | 80 |
| basal and | | | S144D | | *, predicated |
| similar IC₅₀ | | T3175 | S196D | | |
| Decreased | N405Q | D330A | S183A | D330A-N405Q | Bold, Tested in |
| basal but | | | | | T cells |
| higher IC₅₀ | ⁴⁰²GCFNF⁴⁰⁶ISAQT | D330E | S195A | D330A-S144A | |
| | (Casp-10) (SEQ ID | D330G | S196A | D330A-S144D | |
| | NOS 297 and 303) | | | | |
| | F404Y | | | | |
| | F406A | D330N | | D330A-S183A | |
| | F406W | D330S | | D330A-S196A | |
| | F406Y | D330V | | N405Q-S144A | |
| | N405Qco | L329E | | N405Q-S144D | |
| | | T317A | | N405Q-S196D | |
| | | | | N405Q-T317S | |
| | | | | *N405Q-S144Aco | |
| Decreased | F404T | D315A | Y153A | *N405Q-T317Sco | |
| basal but | F404W | A316G | Y153F | | |
| much higher | N405F | F319W | S307A | | |
| IC₅₀ | F406T | | | | |
| Similar basal | C403A | ³¹⁶ATPF³¹⁹AVPI | | | |
| and IC₅₀ | | (SMAC/Diablo) | | | |
| | | (SEQ ID NOS | | | |
| | | 299 and 304) | | | |
| | C403S | T317C | | | |
| | C403T | P318A | | | |
| | N405A | F319A | | | |
| Increased | N405T | T317E | | | D330A-N405T |
| basal | | F326K | | | |
| | | D327G | | | |
| | | D327K | | | |
| | | D327R | | | |
| | | Q328K | | | |
| | | Q328R | | | |
| | | L329G | | | |
| | | L329K | | | |
| | | A331K | | | |
| Catalytically | ⁴⁰²GCFNF⁴⁰⁶AAAAA | | | | C285A |
| dead | (SEQ ID NOS 297 and | | | | |
| | 302) | | | | |
| | ⁴⁰²GCFNF⁴⁰⁶YCSTL | | | | D315A-D330A |
| | (Casp-2) | | | | |
| | (SEQ ID NOS 297 and | | | | |
| | 305) | | | | |
| | ⁴⁰²GCFNF⁴⁰⁶CIVSM | | | | D330A-Y153A |
| | (Casp-3) | | | | |
| | (SEQ ID NOS 297 and | | | | |
| | 306) | | | | |
| | ⁴⁰²GCFNF⁴⁰⁶QPTFT | | | | D330A-Y153F |
| | (Casp-8) | | | | |
| | (SEQ ID NOS 297 and | | | | |
| | 307) | | | | |
| | G402A | | | | D330A-T317E |
| | G402I | | | | |
| | G402Q | | | | |
| | G402Y | | | | |
| | C403P | | | | |
| | F404A | | | | |
| | F404S | | | | |
| | F406L | | | | |

### LITERATURE REFERENCES CITED IN EXAMPLES 6-9

1. Seifert, R. and K. Wenzel-Seifert, Constitutive activity of G-protein-coupled receptors: cause of disease and common property of wild-type receptors. Naunyn Schmiedebergs Arch Pharmacol, 2002. 366(5): p. 381-416.
2. Roose, J. P., et al., T cell receptor-independent basal signaling via Erk and Abl kinases suppresses RAG gene expression. PLoS Biol, 2003. 1(2): p. E53.
3. Tze, L. E., et al., Basal immunoglobulin signaling actively maintains developmental stage in immature B cells. PLoS Biol, 2005. 3(3): p. e82.
4. Schram, B. R., et al., B cell receptor basal signaling regulates antigen-induced Ig light chain rearrangements. J Immunol, 2008. 180(7): p. 4728-41.
5. Randall, K. L., et al., Dock8 mutations cripple B cell immunological synapses, germinal centers and long-lived antibody production. Nat Immunol, 2009. 10(12): p. 1283-91.
6. Kouskoff, V., et al., B cell receptor expression level determines the fate of developing B lymphocytes: receptor editing versus selection. Proc Natl Acad Sci USA, 2000. 97(13): p. 7435-9.
7. Hong, T., et al., A simple theoretical framework for understanding heterogeneous differentiation of CD4⁺ T cells. BMC Syst Biol, 2012. 6: p. 66.
8. Rudd, M. L., A. Tua-Smith, and D. B. Straus, Lck SH3 domain function is required for T-cell receptor signals regulating thymocyte development. Mol Cell Biol, 2006. 26(21): p. 7892-900.
9. Sorkin, A. and M. von Zastrow, Endocytosis and signalling: intertwining molecular networks. Nat Rev Mol Cell Biol, 2009. 10(9): p. 609-22.
10. Luning Prak, E. T., M. Monestier, and R. A. Eisenberg, B cell receptor editing in tolerance and autoimmunity. Ann N Y Acad Sci, 2011. 1217: p. 96-121.
11. Boss, W. F., et al., Basal signaling regulates plant growth and development. Plant Physiol, 2010. 154(2): p. 439-43.
12. Tao, Y. X., Constitutive activation of G protein-coupled receptors and diseases: insights into mechanisms of activation and therapeutics. Pharmacol Ther, 2008. 120(2): p. 129-48.
13. Spiegel, A. M., Defects in G protein-coupled signal transduction in human disease. Annu Rev Physiol, 1996. 58: p. 143-70.
14. Shiozaki, E. N., et al., Mechanism of XIAP-mediated inhibition of Caspase-9. Mol Cell, 2003. 11(2): p. 519-27.
15. Renatus, M., et al., Dimer formation drives the activation of the cell death protease Caspase-9. Proc Natl Acad Sci USA, 2001. 98(25): p. 14250-5.
16. Shi, Y., Mechanisms of Caspase activation and inhibition during apoptosis. Mol Cell, 2002. 9(3): p. 459-70.
17. Shiozaki, E. N., J. Chai, and Y. Shi, Oligomerization and activation of Caspase-9, induced by Apaf-1 CARD. Proc Natl Acad Sci USA, 2002. 99(7): p. 4197-202.
18. Straathof, K. C., et al., An inducible Caspase-9 safety switch for T-cell therapy. Blood, 2005. 105(11): p. 4247-54.
19. MacCorkle, R. A., K. W. Freeman, and D. M. Spencer, Synthetic activation of Caspases: artificial death switches. Proc Natl Acad Sci USA, 1998. 95(7): p. 3655-60.
20. Di Stasi, A., et al., Inducible apoptosis as a safety switch for adoptive cell therapy. N Engl J Med, 2011. 365(18): p. 1673-83.
21. Chang, W. C., et al., Modifying ligand-induced and constitutive signaling of the human 5-HT4 receptor. PLoS One, 2007. 2(12): p. e1317.
22. Bloom, J. D. and F. H. Arnold, In the light of directed evolution: pathways of adaptive protein evolution. Proc Natl Acad Sci USA, 2009. 106 Suppl 1: p. 9995-10000.
23. Boatright, K. M. and G. S. Salvesen, Mechanisms of Caspase activation. Curr Opin Cell Biol, 2003. 15(6): p. 725-31.
24. Boatright, K. M., et al., A unified model for apical Caspase activation. Mol Cell, 2003. 11(2): p. 529-41.
25. Chao, Y., et al., Engineering a dimeric Caspase-9: a re-evaluation of the induced proximity model for Caspase activation. PLoS Biol, 2005. 3(6): p. e183.
26. Stennicke, H. R., et al., Caspase-9 can be activated without proteolytic processing. J Biol Chem, 1999. 274(13): p. 8359-62.
27. Brady, S. C., L. A. Allan, and P. R. Clarke, Regulation of Caspase-9 through phosphorylation by protein kinase C zeta in response to hyperosmotic stress. Mol Cell Biol, 2005. 25(23): p. 10543-55.
28. Martin, M. C., et al., Protein kinase A regulates Caspase-9 activation by Apaf-1 downstream of cytochrome c. J Biol Chem, 2005. 280(15): p. 15449-55.
29. Cardone, M. H., et al., Regulation of cell death protease Caspase-9 by phosphorylation. Science, 1998. 282(5392): p. 1318-21.
30. Raina, D., et al., c-Abl tyrosine kinase regulates Caspase-9 autocleavage in the apoptotic response to DNA damage. J Biol Chem, 2005. 280(12): p. 11147-51.
31. Papworth, C., Bauer, J. C., Braman, J. and Wright, D. A., Site-directed mutagenesis in one day with >80% efficiency. Strategies, 1996. 9(3): p. 3-4.
32. Spencer, D. M., et al., Functional analysis of Fas signaling in vivo using synthetic inducers of dimerization. Curr Biol, 1996. 6(7): p. 839-47.
33. Hsiao, E. C., et al., Constitutive Gs activation using a single-construct tetracycline-inducible expression system in embryonic stem cells and mice. Stem Cell Res Ther, 2011. 2(2): p. 11.
34. Waldner, C., et al., Double conditional human embryonic kidney cell line based on FLP and PhiC31 mediated transgene integration. BMC Res Notes, 2011. 4: p. 420.

The chimeric caspase polypeptides may include amino acid substitutions, including amino acid substitutions that result in a caspase polypeptide with lower basal activity. These may include, for example, iCasp9 D330A, iCasp9 N405Q, and iCasp9 D330A N405Q, demonstrated low to undetectable basal activity, respectively, with a minimum deleterious effect on their AP1903 IC₅₀ in a SEAP reporter-based, surrogate killing assay.

### Example 10: Examples of Particular Nucleic Acid and Amino Acid Sequences

The following is nucleotide sequences provide an example of a construct that may be used for expression of the chimeric protein and CD19 marker. The figure presents the SFG.iC9.2A.²CD19.gcs construct

SEQ ID NO: 1, nucleotide sequence of 5′LTR sequence
TGAAAGACCCCACCTGTAGGTTTGGCAAGCTAGCTTAAGTAACGCCATTTTGCAAGGCATG
GAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGA
ACAGATGGAACAGCTGAATATGGGCCAAACAGGATATCTGTGGTAAGCAGTTCCTGCCCC
GGCTCAGGGCCAAGAACAGATGGTCCCCAGATGCGGTCCAGCCCTCAGCAGTTTCTAGAG
AACCATCAGATGTTTCCAGGGTGCCCCAAGGACCTGAAATGACCCTGTGCCTTATTTGAAC
TAACCAATCAGTTCGCTTCTCGCTTCTGTTCGCGCGCTTATGCTCCCCGAGCTCAATAAAA
GAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGG
AGGGTCTCCTCTGAGTGATTGACTACCCGTCAGCGGGGGTCTTTCA
SEQ ID NO: 2, nucleotide sequence of Fᵥ (human FKBP12v36)
GGAGTGCAGGTGGAAACCATCTCCCCAGGAGACGGGCGCACCTTCCCCAAGCGCGGCCA
GACCTGCGTGGTGCACTACACCGGGATGCTTGAAGATGGAAAGAAAGTTGATTCCTCCCG
GGACAGAAACAAGCCCTTTAAGTTTATGCTAGGCAAGCAGGAGGTGATCCGAGGCTGGGA
AGAAGGGGTTGCCCAGATGAGTGTGGGTCAGAGAGCCAAACTGACTATATCTCCAGATTAT
GCCTATGGTGCCACTGGGCACCCAGGCATCATCCCACCACATGCCACTCTCGTCTTCGAT
GTGGAGCTTCTAAAACTGGAA
SEQ ID NO: 3 amino acid sequence of Fv (human FKBP12v36)
G V Q V E T I S P G D G R T F P K R G Q T C V V H Y T G M L E D G K
K V D S S R D R N K P F K F M L G K Q E V I R G W E E G V A Q M S V
G Q R A K L T I S P D Y A Y G A T G H P G I I P P H A T L V F D V E L L
K L E
SEQ ID NO: 4, GS linker (SEQ ID NO: 151) nucleotide sequence
TCTGGCGGTGGATCCGGA
SEQ ID NO: 5, GS linker (SEQ ID NO: 151) amino acid sequence
S G G G S G
SEQ ID NO: 6, linker nucleotide sequence (between GS linker (SEQ ID NO: 151) and Casp 9)
GTCGAC
SEQ ID NO: 7, linker amino acid sequence (between GS linker (SEQ ID NO: 151) and Casp 9)
VD
SEQ ID NO: 8, Casp 9 (truncated) nucleotide sequence
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCAGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGACGCCATATCTAGTTT
GCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTAATTTCCTCCGGAAAAAACTTTTCTTTAAAACATCA
SEQ ID NO: 9, Caspase-9 (truncated) amino acid sequence-CARD domain deleted
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A Q Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L D A I S S L P T P S D I F V S
Y S T F P G F V S W R D P K S G S W Y V E T L D D I F E Q W A H S E
D L Q S L L L R V A N A V S V K G I Y K Q M P G C F N F L R K K L F F
K T S
SEQ ID NO: 10, linker nucleotide sequence (between Caspase-9 and 2A)
GCTAGCAGA
SEQ ID NO: 11, linker amino acid sequence (between Caspase-9 and 2A)
ASR
SEQ ID NO: 12, Thosea asigna virus-2A from capsid protein precursor nucleotide sequence
GCCGAGGGCAGGGGAAGTCTTCTAACATGCGGGGACGTGGAGGAAAATCCCGGGCCC
SEQ ID NO: 13, Thosea asigna virus-2A from capsid protein precursor amino acid sequence
A E G R G S L L T C G D V E E N P G P
SEQ ID NO: 14, human CD19 (Δcytoplasmic domain) nucleotide sequence (transmembrane
domain in bold)
ATGCCACCTCCTCGCCTCCTCTTCTTCCTCCTCTTCCTCACCCCCATGGAAGTCAGGCCCG
AGGAACCTCTAGTGGTGAAGGTGGAAGAGGGAGATAACGCTGTGCTGCAGTGCCTCAAGG
GGACCTCAGATGGCCCCACTCAGCAGCTGACCTGGTCTCGGGAGTCCCCGCTTAAACCCT
TCTTAAAACTCAGCCTGGGGCTGCCAGGCCTGGGAATCCACATGAGGCCCCTGGCCATCT
GGCTTTTCATCTTCAACGTCTCTCAACAGATGGGGGGCTTCTACCTGTGCCAGCCGGGGC
CCCCCTCTGAGAAGGCCTGGCAGCCTGGCTGGACAGTCAATGTGGAGGGCAGCGGGGAG
CTGTTCCGGTGGAATGTTTCGGACCTAGGTGGCCTGGGCTGTGGCCTGAAGAACAGGTCC
TCAGAGGGCCCCAGCTCCCCTTCCGGGAAGCTCATGAGCCCCAAGCTGTATGTGTGGGC
CAAAGACCGCCCTGAGATCTGGGAGGGAGAGCCTCCGTGTCTCCCACCGAGGGACAGCC
TGAACCAGAGCCTCAGCCAGGACCTCACCATGGCCCCTGGCTCCACACTCTGGCTGTCCT
GTGGGGTACCCCCTGACTCTGTGTCCAGGGGCCCCCTCTCCTGGACCCATGTGCACCCCA
AGGGGCCTAAGTCATTGCTGAGCCTAGAGCTGAAGGACGATCGCCCGGCCAGAGATATGT
GGGTAATGGAGACGGGTCTGTTGTTGCCCCGGGCCACAGCTCAAGACGCTGGAAAGTATT
ATTGTCACCGTGGCAACCTGACCATGTCATTCCACCTGGAGATCACTGCTCGGCCAGTACT
ATGGCACTGGCTGCTGAGGACTGGTGGCTGGAAGGTCTCAGCTGTGACTTTGGCTTATCT
GATCTTCTGCCTGTGTTCCCTTGTGGGCATTCTTCATCTTCAAAGAGCCCTGGTCCTGAGG
AGGAAAAGAAAGCGAATGACTGACCCCACCAGGAGATTC
SEQ ID NO: 15, human CD19 (Δcytoplasmic domain) amino acid sequence
M P P P R L L F F L L F L T P M E V R P E E P L V V K V E E G D N A V
L Q C L K G T S D G P T Q Q L T W S R E S P L K P F L K L S L G L P G
L G I H M R P L A I W L F I F N V S Q Q M G G F Y L C Q P G P P S E K
A W Q P G W T V N V E G S G E L F R W N V S D L G G L G C G L K N
R S S E G P S S P S G K L M S P K L Y V W A K D R P E I W E G E P P
C L P P R D S L N Q S L S Q D L T M A P G S T L W L S C G V P P D S
V S R G P L S W T H V H P K G P K S L L S L E L K D D R P A R D M W
V M E T G L L L P R A T A Q D A G K Y Y C H R G N L T M S F H L E I T
A R P V L W H W L L R T G G W K V S A V T L A Y L I F C L C S L V G I
L H L Q R A L V L R R K R K R M T D P T R R F
SEQ ID NO: 16, 3′LTR nucleotide sequence
TGAAAGACCCCACCTGTAGGTTTGGCAAGCTAGCTTAAGTAACGCCATTTTGCAAGGCATG
GAAAAATACATAACTGAGAATAGAGAAGTTCAGATCAAGGTCAGGAACAGATGGAACAGCT
GAATATGGGCCAAACAGGATATCTGTGGTAAGCAGTTCCTGCCCCGGCTCAGGGCCAAGA
ACAGATGGAACAGCTGAATATGGGCCAAACAGGATATCTGTGGTAAGCAGTTCCTGCCCC
GGCTCAGGGCCAAGAACAGATGGTCCCCAGATGCGGTCCAGCCCTCAGCAGTTTCTAGAG
AACCATCAGATGTTTCCAGGGTGCCCCAAGGACCTGAAATGACCCTGTGCCTTATTTGAAC
TAACCAATCAGTTCGCTTCTCGCTTCTGTTCGCGCGCTTCTGCTCCCCGAGCTCAATAAAA
GAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGG
AGGGTCTCCTCTGAGTGATTGACTACCCGTCAGCGGGGGTCTTTCA
SEQ ID NO: 17, Expression vector construct nucleotide sequence-nucleotide sequence coding
for the chimeric protein and 5′ and 3′ LTR sequences, and additional vector sequence.
TGAAAGACCCCACCTGTAGGTTTGGCAAGCTAGCTTAAGTAACGCCATTTTGCAAGGCATG
GAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGA
ACAGATGGAACAGCTGAATATGGGCCAAACAGGATATCTGTGGTAAGCAGTTCCTGCCCC
GGCTCAGGGCCAAGAACAGATGGTCCCCAGATGCGGTCCAGCCCTCAGCAGTTTCTAGAG
AACCATCAGATGTTTCCAGGGTGCCCCAAGGACCTGAAATGACCCTGTGCCTTATTTGAAC
TAACCAATCAGTTCGCTTCTCGCTTCTGTTCGCGCGCTTATGCTCCCCGAGCTCAATAAAA
GAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGG
AGGGTCTCCTCTGAGTGATTGACTACCCGTCAGCGGGGGTCTTTCATTTGGGGGCTCGTC
CGGGATCGGGAGACCCCTGCCCAGGGACCACCGACCCACCACCGGGAGGTAAGCTGGC
CAGCAACTTATCTGTGTCTGTCCGATTGTCTAGTGTCTATGACTGATTTTATGCGCCTGCGT
CGGTACTAGTTAGCTAACTAGCTCTGTATCTGGCGGACCCGTGGTGGAACTGACGAGTTC
GGAACACCCGGCCGCAACCCTGGGAGACGTCCCAGGGACTTCGGGGGCCGTTTTTGTGG
CCCGACCTGAGTCCTAAAATCCCGATCGTTTAGGACTCTTTGGTGCACCCCCCTTAGAGGA
GGGATATGTGGTTCTGGTAGGAGACGAGAACCTAAAACAGTTCCCGCCTCCGTCTGAATTT
TTGCTTTCGGTTTGGGACCGAAGCCGCGCCGCGCGTCTTGTCTGCTGCAGCATCGTTCTG
TGTTGTCTCTGTCTGACTGTGTTTCTGTATTTGTCTGAAAATATGGGCCCGGGCTAGCCTGT
TACCACTCCCTTAAGTTTGACCTTAGGTCACTGGAAAGATGTCGAGCGGATCGCTCACAAC
CAGTCGGTAGATGTCAAGAAGAGACGTTGGGTTACCTTCTGCTCTGCAGAATGGCCAACCT
TTAACGTCGGATGGCCGCGAGACGGCACCTTTAACCGAGACCTCATCACCCAGGTTAAGA
TCAAGGTCTTTTCACCTGGCCCGCATGGACACCCAGACCAGGTGGGGTACATCGTGACCT
GGGAAGCCTTGGCTTTTGACCCCCCTCCCTGGGTCAAGCCCTTTGTACACCCTAAGCCTC
CGCCTCCTCTTCCTCCATCCGCCCCGTCTCTCCCCCTTGAACCTCCTCGTTCGACCCCGC
CTCGATCCTCCCTTTATCCAGCCCTCACTCCTTCTCTAGGCGCCCCCATATGGCCATATGA
GATCTTATATGGGGCACCCCCGCCCCTTGTAAACTTCCCTGACCCTGACATGACAAGAGTT
ACTAACAGCCCCTCTCTCCAAGCTCACTTACAGGCTCTCTACTTAGTCCAGCACGAAGTCT
GGAGACCTCTGGCGGCAGCCTACCAAGAACAACTGGACCGACCGGTGGTACCTCACCCTT
ACCGAGTCGGCGACACAGTGTGGGTCCGCCGACACCAGACTAAGAACCTAGAACCTCGCT
GGAAAGGACCTTACACAGTCCTGCTGACCACCCCCACCGCCCTCAAAGTAGACGGCATCG
CAGCTTGGATACACGCCGCCCACGTGAAGGCTGCCGACCCCGGGGGTGGACCATCCTCT
AGACTGCCATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAAGATGGAAAG
AAAGTTGATTCCTCCCGGGACAGAAACAAGCCCTTTAAGTTTATGCTAGGCAAGCAGGAGG
TGATCCGAGGCTGGGAAGAAGGGGTTGCCCAGATGAGTGTGGGTCAGAGAGCCAAACTG
ACTATATCTCCAGATTATGCCTATGGTGCCACTGGGCACCCAGGCATCATCCCACCACATG
CCACTCTCGTCTTCGATGTGGAGCTTCTAAAACTGGAATCTGGCGGTGGATCCGGAGTCG
ACGGATTTGGTGATGTCGGTGCTCTTGAGAGTTTGAGGGGAAATGCAGATTTGGCTTACAT
CCTGAGCATGGAGCCCTGTGGCCACTGCCTCATTATCAACAATGTGAACTTCTGCCGTGAG
TCCGGGCTCCGCACCCGCACTGGCTCCAACATCGACTGTGAGAAGTTGCGGCGTCGCTTC
TCCTCGCTGCATTTCATGGTGGAGGTGAAGGGCGACCTGACTGCCAAGAAAATGGTGCTG
GCTTTGCTGGAGCTGGCGCAGCAGGACCACGGTGCTCTGGACTGCTGCGTGGTGGTCAT
TCTCTCTCACGGCTGTCAGGCCAGCCACCTGCAGTTCCCAGGGGCTGTCTACGGCACAGA
TGGATGCCCTGTGTCGGTCGAGAAGATTGTGAACATCTTCAATGGGACCAGCTGCCCCAG
CCTGGGAGGGAAGCCCAAGCTCTTTTTCATCCAGGCCTGTGGTGGGGAGCAGAAAGACCA
TGGGTTTGAGGTGGCCTCCACTTCCCCTGAAGACGAGTCCCCTGGCAGTAACCCCGAGCC
AGATGCCACCCCGTTCCAGGAAGGTTTGAGGACCTTCGACCAGCTGGACGCCATATCTAG
TTTGCCCACACCCAGTGACATCTTTGTGTCCTACTCTACTTTCCCAGGTTTTGTTTCCTGGA
GGGACCCCAAGAGTGGCTCCTGGTACGTTGAGACCCTGGACGACATCTTTGAGCAGTGGG
CTCACTCTGAAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTTTAAAACATC
AGCTAGCAGAGCCGAGGGCAGGGGAAGTCTTCTAACATGCGGGGACGTGGAGGAAAATC
CCGGGCCCATGCCACCTCCTCGCCTCCTCTTCTTCCTCCTCTTCCTCACCCCCATGGAAGT
CAGGCCCGAGGAACCTCTAGTGGTGAAGGTGGAAGAGGGAGATAACGCTGTGCTGCAGT
GCCTCAAGGGGACCTCAGATGGCCCCACTCAGCAGCTGACCTGGTCTCGGGAGTCCCCG
CTTAAACCCTTCTTAAAACTCAGCCTGGGGCTGCCAGGCCTGGGAATCCACATGAGGCCC
CTGGCCATCTGGCTTTTCATCTTCAACGTCTCTCAACAGATGGGGGGCTTCTACCTGTGCC
AGCCGGGGCCCCCCTCTGAGAAGGCCTGGCAGCCTGGCTGGACAGTCAATGTGGAGGGC
AGCGGGGAGCTGTTCCGGTGGAATGTTTCGGACCTAGGTGGCCTGGGCTGTGGCCTGAA
GAACAGGTCCTCAGAGGGCCCCAGCTCCCCTTCCGGGAAGCTCATGAGCCCCAAGCTGTA
TGTGTGGGCCAAAGACCGCCCTGAGATCTGGGAGGGAGAGCCTCCGTGTCTCCCACCGA
GGGACAGCCTGAACCAGAGCCTCAGCCAGGACCTCACCATGGCCCCTGGCTCCACACTCT
GGCTGTCCTGTGGGGTACCCCCTGACTCTGTGTCCAGGGGCCCCCTCTCCTGGACCCATG
TGCACCCCAAGGGGCCTAAGTCATTGCTGAGCCTAGAGCTGAAGGACGATCGCCCGGCC
AGAGATATGTGGGTAATGGAGACGGGTCTGTTGTTGCCCCGGGCCACAGCTCAAGACGCT
GGAAAGTATTATTGTCACCGTGGCAACCTGACCATGTCATTCCACCTGGAGATCACTGCTC
GGCCAGTACTATGGCACTGGCTGCTGAGGACTGGTGGCTGGAAGGTCTCAGCTGTGACTT
TGGCTTATCTGATCTTCTGCCTGTGTTCCCTTGTGGGCATTCTTCATCTTCAAAGAGCCCTG
GTCCTGAGGAGGAAAAGAAAGCGAATGACTGACCCCACCAGGAGATTCTAACGCGTCATC
ATCGATCCGGATTAGTCCAATTTGTTAAAGACAGGATATCAGTGGTCCAGGCTCTAGTTTTG
ACTCAACAATATCACCAGCTGAAGCCTATAGAGTACGAGCCATAGATAAAATAAAAGATTTT
ATTTAGTCTCCAGAAAAAGGGGGGAATGAAAGACCCCACCTGTAGGTTTGGCAAGCTAGCT
TAAGTAACGCCATTTTGCAAGGCATGGAAAAATACATAACTGAGAATAGAGAAGTTCAGATC
AAGGTCAGGAACAGATGGAACAGCTGAATATGGGCCAAACAGGATATCTGTGGTAAGCAG
TTCCTGCCCCGGCTCAGGGCCAAGAACAGATGGAACAGCTGAATATGGGCCAAACAGGAT
ATCTGTGGTAAGCAGTTCCTGCCCCGGCTCAGGGCCAAGAACAGATGGTCCCCAGATGCG
GTCCAGCCCTCAGCAGTTTCTAGAGAACCATCAGATGTTTCCAGGGTGCCCCAAGGACCT
GAAATGACCCTGTGCCTTATTTGAACTAACCAATCAGTTCGCTTCTCGCTTCTGTTCGCGC
GCTTCTGCTCCCCGAGCTCAATAAAAGAGCCCACAACCCCTCACTCGGGGCGCCAGTCCT
CCGATTGACTGAGTCGCCCGGGTACCCGTGTATCCAATAAACCCTCTTGCAGTTGCATCCG
ACTTGTGGTCTCGCTGTTCCTTGGGAGGGTCTCCTCTGAGTGATTGACTACCCGTCAGCG
GGGGTCTTTCACACATGCAGCATGTATCAAAATTAATTTGGTTTTTTTTCTTAAGTATTTACA
TTAAATGGCCATAGTACTTAAAGTTACATTGGCTTCCTTGAAATAAACATGGAGTATTCAGA
ATGTGTCATAAATATTTCTAATTTTAAGATAGTATCTCCATTGGCTTTCTACTTTTTCTTTTAT
TTTTTTTTGTCCTCTGTCTTCCATTTGTTGTTGTTGTTGTTTGTTTGTTTGTTTGTTGGTTGGT
TGGTTAATTTTTTTTTAAAGATCCTACACTATAGTTCAAGCTAGACTATTAGCTACTCTGTAA
CCCAGGGTGACCTTGAAGTCATGGGTAGCCTGCTGTTTTAGCCTTCCCACATCTAAGATTA
CAGGTATGAGCTATCATTTTTGGTATATTGATTGATTGATTGATTGATGTGTGTGTGTGTGA
TTGTGTTTGTGTGTGTGACTGTGAAAATGTGTGTATGGGTGTGTGTGAATGTGTGTATGTAT
GTGTGTGTGTGAGTGTGTGTGTGTGTGTGTGCATGTGTGTGTGTGTGACTGTGTCTATGTG
TATGACTGTGTGTGTGTGTGTGTGTGTGTGTGTGTGTGTGTGTGTGTGTGTGTTGTGAAAA
AATATTCTATGGTAGTGAGAGCCAACGCTCCGGCTCAGGTGTCAGGTTGGTTTTTGAGACA
GAGTCTTTCACTTAGCTTGGAATTCACTGGCCGTCGTTTTACAACGTCGTGACTGGGAAAA
CCCTGGCGTTACCCAACTTAATCGCCTTGCAGCACATCCCCCTTTCGCCAGCTGGCGTAAT
AGCGAAGAGGCCCGCACCGATCGCCCTTCCCAACAGTTGCGCAGCCTGAATGGCGAATG
GCGCCTGATGCGGTATTTTCTCCTTACGCATCTGTGCGGTATTTCACACCGCATATGGTGC
ACTCTCAGTACAATCTGCTCTGATGCCGCATAGTTAAGCCAGCCCCGACACCCGCCAACAC
CCGCTGACGCGCCCTGACGGGCTTGTCTGCTCCCGGCATCCGCTTACAGACAAGCTGTGA
CCGTCTCCGGGAGCTGCATGTGTCAGAGGTTTTCACCGTCATCACCGAAACGCGCGATGA
CGAAAGGGCCTCGTGATACGCCTATTTTTATAGGTTAATGTCATGATAATAATGGTTTCTTA
GACGTCAGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAATAATATTGA
AAAAGGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGATCAG
TTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGT
ATTATCCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAACTCG
CCTTGATCGTTGGGAACCGGAGCTGAATGAAGCCATACCAAACGACGAGCGTGACACCAC
GATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTATCGTAGTTATCT
ACACGACGGGGAGTCAGGCAACTATGGATGAACGAAATAGACAGATCGCTGAGATAGGTG
CCTCACTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCATGACCA
AAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGAAGGTAACTGG
CTTCAGCAGAGCGCAGATACCAAATACTGTCCTTCTAGTGTAGCCGTAGTTAGGCCACCAC
TTCAAGAACTCTGTAGCACCGCCTACATACCTCGCTCTGCTAATCCTGTTACCAGTGGCTG
CTGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAAC
GACCTACACCGAACTGAGATACCTACAGCGTGAGCATTGAGAAAGCGCCACGCTTCCCGA
AGGGAGAAAGGCGGACAGGTATCCGGTAAGCGGCAGGGTCGGAACAGGAGAGCGCACG
AGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATACCGCT
CGCCGCAGCCGAACGACCGAGCGCAGCGAGTCAGTGAGCGAGGAAGCGGAAGAGCGCC
CAATACGCAAACCGCCTCTCCCCGCGCGTTGGCCGATTCATTAATGCAGCTGGCACGACA
GGTTTCCCGACTGGAAAGCGGGCAGTGAGCGCAACGCAATTAATGTGAGTTAGCTCACTC
ATTAGGCACCCCAGGCTTTACACTTTATGCTTCCGGCTCGTATGTTGTGTGGAATTGTGAG
CGGATAACAATTTCACACAGGAAACAGCTATGACCATGATTACGCCAAGCTTTGCTCTTAG
GAGTTTCCTAATACATCCCAAACTCAAATATATAAAGCATTTGACTTGTTCTATGCCCTAGG
GGGCGGGGGGAAGCTAAGCCAGCTTTTTTTAACATTTAAAATGTTAATTCCATTTTAAATGC
ACAGATGTTTTTATTTCATAAGGGTTTCAATGTGCATGAATGCTGCAATATTCCTGTTACCAA
AGCTAGTATAAATAAAAATAGATAAACGTGGAAATTACTTAGAGTTTCTGTCATTAACGTTTC
CTTCCTCAGTTGACAACATAAATGCGCTGCTGAGCAAGCCAGTTTGCATCTGTCAGGATCA
ATTTCCCATTATGCCAGTCATATTAATTACTAGTCAATTAGTTGATTTTTATTTTTGACATATA
CATGTGAA
SEQ ID NO: 18, (nucleotide sequence of F_{v′}Fᵥₗₛ with Xhol/Sall linkers, (wobbled codons
lowercase in F_{v′}))
ctcgagGGcGTcCAaGTcGAaACcATtagtCCcGGcGAtGGcaGaACaTTtCCtAAaaGgGGaCAaACa
TGtGTcGTcCAtTAtACaGGcATGtTgGAgGAcGGcAAaAAgGTgGAcagtagtaGaGAtcGcAAtAAaC
CtTTcAAaTTcATGtTgGGaAAaCAaGAaGTcATtaGgGGaTGGGAgGAgGGcGTgGCtCAaATGtcc
GTcGGcCAacGcGCtAAgCTcACcATcagcCCcGAcTAcGCaTAcGGcGCtACcGGaCAtCCcGGaA
TtATtCCcCCtCAcGCtACctTgGTgTTtGAcGTcGAaCTgtTgAAgCTcGAagtcgagggagtgcaggtggaa
accatctccccaggagacgggcgcaccttccccaagcgcggccagacctgcgtggtgcactacaccgggatgcttgaagatggaa
agaaagttgattcctcccgggacagaaacaagccctttaagtttatgctaggcaagcaggaggtgatccgaggctgggaagaaggg
gttgcccagatgagtgtgggtcagagagccaaactgactatatctccagattatgcctatggtgccactgggcacccaggcatcatccc
accacatgccactctcgtcttcgatgtggagcttctaaaactggaatctggcggtggatccggagtcgag
SEQ ID NO: 19, (F_{V'}F_{VLS} amino acid sequence)
GlyValGlnValGluThrIleSerProGlyAspGlyArgThrPheProLysArgGlyGlnThrCysValValHisTyrThrGly
MetLeuGluAspGlyLysLysValAspSerSerArgAspArgAsnLysProPheLysPheMetLeuGlyLysGlnGluVal
IleArgGlyTrpGluGluGlyValAlaGlnMetSerValGlyGlnArgAlaLysLeuThrIleSerProAspTyrAlaTyrGl
yAlaThrGlyHisProGlyIleIleProProHisAlaThrLeuValPheAspValGluLeuLeuLysLeuGlu(ValGlu)
GlyValGlnValGluThrIleSerProGlyAspGlyArgThrPheProLysArgGlyGlnThrCysValValHisTyrThrGly
MetLeuGluAspGlyLysLysValAspSerSerArgAspArgAsnLysProPheLysPheMetLeuGlyLysGlnGluVal
IleArgGlyTrpGluGluGlyValAlaGlnMetSerValGlyGlnArgAlaLysLeuThrIleSerProAspTyrAlaTyrGl
yAlaThrGlyHisProGlyIleIleProProHisAlaThrLeuValPheAspValGluLeuLeuLysLeuGlu-
SerGlyGlyGlySerGly
SEQ ID NO: 20, FKBP12v36 (res. 2-108)
SGGGSG Linker (6 aa) (SEQ ID NO: 151)
ΔCasp9 (res. 135-416)
ATGCTCGAGGGAGTGCAGGTGGAgACtATCTCCCCAGGAGACGGGCGCACCTTCCCCAAG
CGCGGCCAGACCTGCGTGGTGCACTACACCGGGATGCTTGAAGATGGAAAGAAAGTTGAT
TCCTCCCGGGACAGAAACAAGCCCTTTAAGTTTATGCTAGGCAAGCAGGAGGTGATCCGA
GGCTGGGAAGAAGGGGTTGCCCAGATGAGTGTGGGTCAGAGAGCCAAACTGACTATATCT
CCAGATTATGCCTATGGTGCCACTGGGCACCCAGGCATCATCCCACCACATGCCACTCTC
GTCTTCGATGTGGAGCTTCTAAAACTGGAATCTGGCGGTGGATCCGGAGTCGACGGATTT
GGTGATGTCGGTGCTCTTGAGAGTTTGAGGGGAAATGCAGATTTGGCTTACATCCTGAGCA
TGGAGCCCTGTGGCCACTGCCTCATTATCAACAATGTGAACTTCTGCCGTGAGTCCGGGC
TCCGCACCCGCACTGGCTCCAACATCGACTGTGAGAAGTTGCGGCGTCGCTTCTCCTCGC
TGCATTTCATGGTGGAGGTGAAGGGCGACCTGACTGCCAAGAAAATGGTGCTGGCTTTGC
TGGAGCTGGCGCgGCAGGACCACGGTGCTCTGGACTGCTGCGTGGTGGTCATTCTCTCTC
ACGGCTGTCAGGCCAGCCACCTGCAGTTCCCAGGGGCTGTCTACGGCACAGATGGATGC
CCTGTGTCGGTCGAGAAGATTGTGAACATCTTCAATGGGACCAGCTGCCCCAGCCTGGGA
GGGAAGCCCAAGCTCTTTTTCATCCAGGCCTGTGGTGGGGAGCAGAAAGACCATGGGTTT
GAGGTGGCCTCCACTTCCCCTGAAGACGAGTCCCCTGGCAGTAACCCCGAGCCAGATGC
CACCCCGTTCCAGGAAGGTTTGAGGACCTTCGACCAGCTGGACGCCATATCTAGTTTGCC
CACACCCAGTGACATCTTTGTGTCCTACTCTACTTTCCCAGGTTTTGTTTCCTGGAGGGAC
CCCAAGAGTGGCTCCTGGTACGTTGAGACCCTGGACGACATCTTTGAGCAGTGGGCTCAC
TCTGAAGACCTGCAGTCCCTCCTGCTTAGGGTCGCTAATGCTGTTTCGGTGAAAGGGATTT
ATAAACAGATGCCTGGTTGCTTTAATTTCCTCCGGAAAAAACTTTTCTTTAAAACATCA
SEQ ID NO: 21, FKBP12v36 (res. 2-108)
G V Q V E T I S P G D G R T F P K R G Q T C V V H Y T G M L E D G K
K V D S S R D R N K P F K F M L G K Q E V I R G W E E G V A Q M S V
G Q R A K L T I S P D Y A Y G A T G H P G I I P P H A T L V F D V E L L
K L E
SEQ ID NO: 22, ΔCasp9 (res. 135-416)
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L D A I S S L P T P S D I F V S
Y S T F P G F V S W R D P K S G S W Y V E T L D D I F E Q W A H S E
D L Q S L L L R V A N A V S V K G I Y K Q M P G C F N F L R K K L F F
K T S
SEQ ID NO: 23, ΔCasp9 (res. 135-416) D330A, nucleotide sequence
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGCCGCCATATCTAGTT
TGCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTAATTTCCTCCGGAAAAAACTTTTCTTTAAAACATCA
SEQ ID NO: 24, ΔCasp9 (res. 135-416) D330A, amino acid sequence
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L A A I S S L P T P S D I F V S
Y S T F P G F V S W R D P K S G S W Y V E T L D D I F E Q W A H S E
D L Q S L L L R V A N A V S V K G I Y K Q M P G C F N F L R K K L F F
K T S
SEQ ID NO: 25, ΔCasp9 (res. 135-416) N405Q nucleotide sequence
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGACGCCATATCTAGTTT
GCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTCAGTTCCTCCGGAAAAAACTTTTCTTTAAAACATCA
SEQ ID NO: 26, ΔCasp9 (res. 135-416) N405Q amino acid sequence
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L D A I S S L P T P S D I F V S
Y S T F P G F V S W R D P K S G S W Y V E T L D D I F E Q W A H S E
D L Q S L L L R V A N A V S V K G I Y K Q M P G C F Q F L R K K L F F
K T S
SEQ ID NO: 27, ΔCasp9 (res. 135-416) D330A N405Q nucleotide sequence
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGCCGCCATATCTAGTT
TGCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTCAGTTCCTCCGGAAAAAACTTTTCTTTAAAACATCA
SEQ ID NO: 28, ΔCasp9 (res. 135-416) D330A N405Q amino acid sequence
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L A A I S S L P T P S D I F V S
Y S T F P G F V S W R D P K S G S W Y V E T L D D I F E Q W A H S E
D L Q S L L L R V A N A V S V K G I Y K Q M P G C F Q F L R K K L F F
K T S
SEQ ID NO: 29, FKBPv36 (Fv1) nucleotide sequence
GGCGTTCAAGTAGAAACAATCAGCCCAGGAGACGGAAGGACTTTCCCCAAACGAGGCCAA
ACATGCGTAGTTCATTATACTGGGATGCTCGAAGATGGAAAAAAAGTAGATAGTAGTAGAG
ACCGAAACAAACCATTTAAATTTATGTTGGGAAAACAAGAAGTAATAAGGGGCTGGGAAGA
AGGTGTAGCACAAATGTCTGTTGGCCAGCGCGCAAAACTCACAATTTCTCCTGATTATGCT
TACGGAGCTACCGGCCACCCCGGCATCATACCCCCTCATGCCACACTGGTGTTTGACGTC
GAATTGCTCAAACTGGAA
SEQ ID NO: 30, FKBPv36 (Fv1) amino acid sequence
GVQVETISPGDGRTFPKRGQTCVVHYTGMLEDGKKVDSSRDRNKPFKFMLGKQEVIRGWEE
GVAQMSVGQRAKLTISPDYAYGATGHPGIIPPHATLVFDVELLKLE
SEQ ID NO: 31, FKBPv36 (Fv2) nucleotide sequence
GGaGTgCAgGTgGAgACgATtAGtCCtGGgGAtGGgAGaACcTTtCCaAAgCGcGGtCAgACcTGtG
TtGTcCAcTAcACcGGtATGCTgGAgGAcGGgAAgAAgGTgGActcTtcacGcGAtCGcAAtAAgCCtTT
cAAgTTcATGcTcGGcAAgCAgGAgGTgATccGGGGgTGGGAgGAgGGcGTgGCtCAgATGTCgG
TcGGgCAaCGaGCgAAgCTtACcATcTCaCCcGAcTAcGCgTAtGGgGCaACgGGgCAtCCgGGaA
TtATcCCtCCcCAcGCtACgCTcGTaTTcGAtGTgGAgcTcttgAAgCTtGag
SEQ ID NO: 32, FKBPv36 (Fv2) amino acid sequence
GVQVETISPGDGRTFPKRGQTCVVHYTGMLEDGKKVDSSRDRNKPFKFMLGKQEVIRGWEE
GVAQMSVGQRAKLTISPDYAYGATGHPGIIPPHATLVFDVELLKLE
SEQ ID NO: 33, ΔCD19 nucleotide sequence
ATGCCCCCTCCTAGACTGCTGTTTTTCCTGCTCTTTCTCACCCCAATGGAAGTTAGACCTGA
GGAACCACTGGTCGTTAAAGTGGAAGAAGGTGATAATGCTGTCCTCCAATGCCTTAAAGGG
ACCAGCGACGGACCAACGCAGCAACTGACTTGGAGCCGGGAGTCCCCTCTCAAGCCGTTT
CTCAAGCTGTCACTTGGCCTGCCAGGTCTTGGTATTCACATGCGCCCCCTTGCCATTTGGC
TCTTCATATTCAATGTGTCTCAACAAATGGGTGGATTCTACCTTTGCCAGCCCGGCCCCCC
TTCTGAGAAAGCTTGGCAGCCTGGATGGACCGTCAATGTTGAAGGCTCCGGTGAGCTGTT
TAGATGGAATGTGAGCGACCTTGGCGGACTCGGTTGCGGACTGAAAAATAGGAGCTCTGA
AGGACCCTCTTCTCCCTCCGGTAAGTTGATGTCACCTAAGCTGTACGTGTGGGCCAAGGA
CCGCCCCGAAATCTGGGAGGGCGAGCCTCCATGCCTGCCGCCTCGCGATTCACTGAACC
AGTCTCTGTCCCAGGATCTCACTATGGCGCCCGGATCTACTCTTTGGCTGTCTTGCGGCGT
TCCCCCAGATAGCGTGTCAAGAGGACCTCTGAGCTGGACCCACGTACACCCTAAGGGCCC
TAAGAGCTTGTTGAGCCTGGAACTGAAGGACGACAGACCCGCACGCGATATGTGGGTAAT
GGAGACCGGCCTTCTGCTCCCTCGCGCTACCGCACAGGATGCAGGGAAATACTACTGTCA
TAGAGGGAATCTGACTATGAGCTTTCATCTCGAAATTACAGCACGGCCCGTTCTTTGGCAT
TGGCTCCTCCGGACTGGAGGCTGGAAGGTGTCTGCCGTAACACTCGCTTACTTGATTTTTT
GCCTGTGTAGCCTGGTTGGGATCCTGCATCTTCAGCGAGCCCTTGTATTGCGCCGAAAAA
GAAAACGAATGACTGACCCTACACGACGATTCTGA
SEQ ID NO: 34, ΔCD19 amino acid sequence
MPPPRLLFFLLFLTPMEVRPEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGVVTVNVEGSGELFRWN
VSDLGGLGCGLKNRSSEGPSSPSGKLMSPKLYVWAKDRPEIWEGEPPCLPPRDSLNQSLSQD
LTMAPGSTLWLSCGVPPDSVSRGPLSVVTHVHPKGPKSLLSLELKDDRPARDMVVVMETGLLLP
RATAQDAGKYYCHRGNLTMSFHLEITARPVLWHWLLRTGGWKVSAVTLAYLIFCLCSLVGILHL
QRALVLRRKRKRMTDPTRRF*

Codon optimized iCasp9-N405Q-2A-ΔCD19 sequence: (the .co following the name of a nucleotide sequence indicates that it is codon optimized (or the amino acid sequence coded by the codon-optimized nucleotide sequence).

SEQ ID NO: 35, FKBPv36.co (Fv3) nucleotide sequence
ATGCTGGAGGGAGTGCAGGTGGAGACTATTAGCCCCGGAGATGGCAGAACATTCCCCAAA
AGAGGACAGACTTGCGTCGTGCATTATACTGGAATGCTGGAAGACGGCAAGAAGGTGGAC
AGCAGCCGGGACCGAAACAAGCCCTTCAAGTTCATGCTGGGGAAGCAGGAAGTGATCCG
GGGCTGGGAGGAAGGAGTCGCACAGATGTCAGTGGGACAGAGGGCCAAACTGACTATTA
GCCCAGACTACGCTTATGGAGCAACCGGCCACCCCGGGATCATTCCCCCTCATGCTACAC
TGGTCTTCGATGTGGAGCTGCTGAAGCTGGAA
SEQ ID NO: 36, FKBPv36.co (Fv3) amino acid sequence
MLEGVQVETISPGDGRTFPKRGQTCVVHYTGMLEDGKKVDSSRDRNKPFKFMLGKQEVIRGW
EEGVAQMSVGQRAKLTISPDYAYGATGHPGIIPPHATLVFDVELLKLE
SEQ ID NO: 37, Linker.co nucleotide sequence
AGCGGAGGAGGATCCGGA
SEQ ID NO: 38, Linker.co amino acid sequence
SGGGSG
SEQ ID NO: 39, Caspase-9.co nucleotide sequence
GTGGACGGGTTTGGAGATGTGGGAGCCCTGGAATCCCTGCGGGGCAATGCCGATCTGGC
TTACATCCTGTCTATGGAGCCTTGCGGCCACTGTCTGATCATTAACAATGTGAACTTCTGCA
GAGAGAGCGGGCTGCGGACCAGAACAGGATCCAATATTGACTGTGAAAAGCTGCGGAGAA
GGTTCTCTAGTCTGCACTTTATGGTCGAGGTGAAAGGCGATCTGACCGCTAAGAAAATGGT
GCTGGCCCTGCTGGAACTGGCTCGGCAGGACCATGGGGCACTGGATTGCTGCGTGGTCG
TGATCCTGAGTCACGGCTGCCAGGCTTCACATCTGCAGTTCCCTGGGGCAGTCTATGGAA
CTGACGGCTGTCCAGTCAGCGTGGAGAAGATCGTGAACATCTTCAACGGCACCTCTTGCC
CAAGTCTGGGCGGGAAGCCCAAACTGTTCTTTATTCAGGCCTGTGGAGGCGAGCAGAAAG
ATCACGGCTTCGAAGTGGCTAGCACCTCCCCCGAGGACGAATCACCTGGAAGCAACCCTG
AGCCAGATGCAACCCCCTTCCAGGAAGGCCTGAGGACATTTGACCAGCTGGATGCCATCT
CAAGCCTGCCCACACCTTCTGACATTTTCGTCTCTTACAGTACTTTCCCTGGATTTGTGAGC
TGGCGCGATCCAAAGTCAGGCAGCTGGTACGTGGAGACACTGGACGATATCTTTGAGCAG
TGGGCCCATTCTGAAGACCTGCAGAGTCTGCTGCTGCGAGTGGCCAATGCTGTCTCTGTG
AAGGGGATCTACAAACAGATGCCAGGATGCTTCCAGTTTCTGAGAAAGAAACTGTTCTTTA
AGACCTCCGCATCTAGGGCC
SEQ ID NO: 40, Caspase-9.co amino acid sequence
VDGFGDVGALESLRGNADLAYILSMEPCGHCLIINNVNFCRESGLRTRTGSNIDCEKLRRRFSS
LHFMVEVKGDLTAKKMVLALLELARQDHGALDCCVVVILSHGCQASHLQFPGAVYGTDGCPVS
VEKIVNIFNGTSCPSLGGKPKLFFIQACGGEQKDHGFEVASTSPEDESPGSNPEPDATPFQEGL
RTFDQLDAISSLPTPSDIFVSYSTFPGFVSWRDPKSGSVVYVETLDDIFEQWAHSEDLQSLLLRV
ANAVSVKGIYKQMPGCFQFLRKKLFFKTSASRA
SEQ ID NO: 41, Linker.co nucleotide sequence
CCGCGG
SEQ ID NO: 42, Linker.co amino acid sequence
PR
SEQ ID NO: 308: T2A.co nucleotide sequence
GAAGGCCGAGGGAGCCTGCTGACATGTGGCGATGTGGAGGAAAACCCAGGACCA
SEQ ID NO: 43: T2A.co amino acid sequence
EGRGSLLTCGDVEENPGP
SEQ ID NO: 309: ΔCD19.co nucleotide sequence
ATGCCACCACCTCGCCTGCTGTTCTTTCTGCTGTTCCTGACACCTATGGAGGTGCGACCTG
AGGAACCACTGGTCGTGAAGGTCGAGGAAGGCGACAATGCCGTGCTGCAGTGCCTGAAA
GGCACTTCTGATGGGCCAACTCAGCAGCTGACCTGGTCCAGGGAGTCTCCCCTGAAGCCT
TTTCTGAAACTGAGCCTGGGACTGCCAGGACTGGGAATCCACATGCGCCCTCTGGCTATC
TGGCTGTTCATCTTCAACGTGAGCCAGCAGATGGGAGGATTCTACCTGTGCCAGCCAGGA
CCACCATCCGAGAAGGCCTGGCAGCCTGGATGGACCGTCAACGTGGAGGGGTCTGGAGA
ACTGTTTAGGTGGAATGTGAGTGACCTGGGAGGACTGGGATGTGGGCTGAAGAACCGCTC
CTCTGAAGGCCCAAGTTCACCCTCAGGGAAGCTGATGAGCCCAAAACTGTACGTGTGGGC
CAAAGATCGGCCCGAGATCTGGGAGGGAGAACCTCCATGCCTGCCACCTAGAGACAGCCT
GAATCAGAGTCTGTCACAGGATCTGACAATGGCCCCCGGGTCCACTCTGTGGCTGTCTTG
TGGAGTCCCACCCGACAGCGTGTCCAGAGGCCCTCTGTCCTGGACCCACGTGCATCCTAA
GGGGCCAAAAAGTCTGCTGTCACTGGAACTGAAGGACGATCGGCCTGCCAGAGACATGTG
GGTCATGGAGACTGGACTGCTGCTGCCACGAGCAACCGCACAGGATGCTGGAAAATACTA
TTGCCACCGGGGCAATCTGACAATGTCCTTCCATCTGGAGATCACTGCAAGGCCCGTGCT
GTGGCACTGGCTGCTGCGAACCGGAGGATGGAAGGTCAGTGCTGTGACACTGGCATATCT
GATCTTTTGCCTGTGCTCCCTGGTGGGCATTCTGCATCTGCAGAGAGCCCTGGTGCTGCG
GAGAAAGAGAAAGAGAATGACTGACCCAACAAGAAGGTTTTGA
SEQ ID NO: 310: ΔCD19.co amino acid sequence
MPPPRLLFFLLFLTPMEVRPEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGVVTVNVEGSGELFRWN
VSDLGGLGCGLKNRSSEGPSSPSGKLMSPKLYVWAKDRPEIWEGEPPCLPPRDSLNQSLSQD
LTMAPGSTLWLSCGVPPDSVSRGPLSVVTHVHPKGPKSLLSLELKDDRPARDMVVVMETGLLLP
RATAQDAGKYYCHRGNLTMSFHLEITARPVLWHWLLRTGGWKVSAVTLAYLIFCLCSLVGILHL
QRALVLRRKRKRMTDPTRRF*

TABLE 6
Additional Examples of Caspase-9 Variants

| iCasp9 Variants | DNA sequence | Amino acid sequence |
|---|---|---|
| Fv-L-Caspase9 WT-2A | Fv disclosed as SEQ ID NO: 311, | Fv disclosed as SEQ ID NO: 314, |
| | Linker disclosed as SEQ ID NO: 312, | Linker disclosed as SEQ ID NO: |
| | iCasp9 disclose as SEQ ID NO: 44 | 315, iCasp9 disclose as SEQ ID |
| | and T2A disclosed as SEQ ID NO: 313 | NO: 45 and T2A disclosed as SEQ |
| | (Fv)ATGCTCGAGGGAGTGCAGGTGG | ID NO: 316 |
| | AgACtATCTCCCCAGGAGACGGGCG | (Fv)MLEGVQVETISPGDGRTFPKRG |
| | CACCTTCCCCAAGCGCGGCCAGACC | QTCVVHYTGMLEDGKKVDSSRDRN |
| | TGCGTGGTGCACTACACCGGGATGC | KPFKFMLGKQEVIR |
| | TTGAAGATGGAAAGAAAGTTGATTCC | GWEEGVAQMSVGQRAKLTISPDYA |
| | TCCCGGGACAGAAACAAGCCCTTTA | YGATGHPGIIPPHATLVFDVELLKLE- |
| | AGTTTATGCTAGGCAAGCAGGAGGT | (linker)SGGGSG-(iCasp9)VDGF |
| | GATCCGAGGCTGGGAAGAAGGGGT | GDVGALESLRGNADLAYILSMEPC |
| | TGCCCAGATGAGTGTGGGTCAGAGA | GHCLIINNVNFCRESGLRTRTGSNID |
| | GCCAAACTGACTATATCTCCAGATTA | CEKLRRRFSS |
| | TGCCTATGGTGCCACTGGGCACCCA | LHFMVEVKGDLTAKKMVLALLELAR |
| | GGCATCATCCCACCACATGCCACTC | QDHGALDCCVVVILSHGCQASHLQ |
| | TCGTCTTCGATGTGGAGCTTCTAAAA | FPGAVYGTDGC |
| | CTGGA- | PVSVEKIVNIFNGTSCPSLGGKPKLF |
| | (linker)TCTGGCGGTGGATCCGGA- | FIQACGGEQKDHGFEVASTSPEDE |
| | (iCasp9)GTCGACGGATTTGGTGATGT | SPGSNPEPDA |
| | CGGTGCTCTTGAGAGTTTGAGGGGA | TPFQEGLRTFDQLDAISSLPTPSDIF |
| | AATGCAGATTTGGCTTACATCCTGAG | VSYSTFPGFVSWRDPKSGSVVYVET |
| | CATGGAGCCCTGTGGCCACTGCCTC | LDDIFEQWAH |
| | ATTATCAACAATGTGAACTTCTGCCG | SEDLQSLLLRVANAVSVKGIYKQMP |
| | TGAGTCCGGGCTCCGCACCCGCACT | GCFNFLRKKLFFKTSASRA- |
| | GGCTCCAACATCGACTGTGAGAAGT | EGRGSLLTCGDVEENP |
| | TGCGGCGTCGCTTCTCCTCGCTGCA | GP- |
| | TTTCATGGTGGAGGTGAAGGGCGAC | |
| | CTGACTGCCAAGAAAATGGTGCTGG | |
| | CTTTGCTGGAGCTGGCGCGGCAGG | |
| | ACCACGGTGCTCTGGACTGCTGCGT | |
| | GGTGGTCATTCTCTCTCACGGCTGT | |
| | CAGGCCAGCCACCTGCAGTTCCCAG | |
| | GGGCTGTCTACGGCACAGATGGATG | |
| | CCCTGTGTCGGTCGAGAAGATTGTG | |
| | AACATCTTCAATGGGACCAGCTGCC | |
| | CCAGCCTGGGAGGGAAGCCCAAGC | |
| | TCTTTTTCATCCAGGCCTGTGGTGG | |
| | GGAGCAGAAAGACCATGGGTTTGAG | |
| | GTGGCCTCCACTTCCCCTGAAGACG | |
| | AGTCCCCTGGCAGTAACCCCGAGCC | |
| | AGATGCCACCCCGTTCCAGGAAGGT | |
| | TTGAGGACCTTCGACCAGCTGGACG | |
| | CCATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A)- | |
| | GAGGGCAGGGGAAGTCTTCTAACAT | |
| | GCGGGGACGTGGAGGAAAATCCCG | |
| | GGCCC | |
| Fv-L-iCaspase9 WT | Fv disclosed as SEQ ID NO: 317, | iCaspase9 disclosed as SEQ ID |
| codon optimized-T2A | Linker disclosed as SEQ ID NO: 318, | NO: 47 and T2A disclosed as SEQ |
| codon optimized | iCasp9 disclose as SEQ ID NO: 46 | ID NO: 320 |
| | and T2A disclosed as SEQ ID NO: 319 | (Fv-L)- |
| | (Fv)- | VDGFGDVGALESLRGNADLAYILSM |
| | GGAGTGCAGGTGGAGACTATTAGCC | EPCGHCLIINNVNFCRESGLRTRTG |
| | CCGGAGATGGCAGAACATTCCCCAA | SNIDCEKLRRRFSS |
| | AAGAGGACAGACTTGCGTCGTGCAT | LHFMVEVKGDLTAKKMVLALLELAR |
| | TATACTGGAATGCTGGAAGACGGCA | QDHGALDCCVVVILSHGCQASHLQ |
| | AGAAGGTGGACAGCAGCCGGGACC | FPGAVYGTDGC |
| | GAAACAAGCCCTTCAAGTTCATGCT | PVSVEKIVNIFNGTSCPSLGGKPKLF |
| | GGGGAAGCAGGAAGTGATCCGGGG | FIQACGGEQKDHGFEVASTSPEDE |
| | CTGGGAGGAAGGAGTCGCACAGAT | SPGSNPEPDA |
| | GTCAGTGGGACAGAGGGCCAAACT | TPFQEGLRTFDQLDAISSLPTPSDIF |
| | GACTATTAGCCCAGACTACGCTTAT | VSYSTFPGFVSWRDPKSGSVVYVET |
| | GGAGCAACCGGCCACCCCGGGATC | LDDIFEQWAH |
| | ATTCCCCCTCATGCTACACTGGTCTT | SEDLQSLLLRVANAVSVKGIYKQMP |
| | CGATGTGGAGCTGCTGAAGCTGGAA- | GCFNFLRKKLFFKTSASRA- |
| | (L)-AGCGGAGGAGGATCCGGA- | EGRGSLLTCGDVEENP |
| | (iCasp9)- | GP-(T2A) |
| | GTGGACGGGTTTGGAGATGTGGGA | |
| | GCCCTGGAATCCCTGCGGGGCAAT | |
| | GCCGATCTGGCTTACATCCTGTCTAT | |
| | GGAGCCTTGCGGCCACTGTCTGATC | |
| | ATTAACAATGTGAACTTCTGCAGAGA | |
| | GAGCGGGCTGCGGACCAGAACAGG | |
| | ATCCAATATTGACTGTGAAAAGCTGC | |
| | GGAGAAGGTTCTCTAGTCTGCACTT | |
| | TATGGTCGAGGTGAAAGGCGATCTG | |
| | ACCGCTAAGAAAATGGTGCTGGCCC | |
| | TGCTGGAACTGGCTCGGCAGGACCA | |
| | TGGGGCACTGGATTGCTGCGTGGTC | |
| | GTGATCCTGAGTCACGGCTGCCAGG | |
| | CTTCACATCTGCAGTTCCCTGGGGC | |
| | AGTCTATGGAACTGACGGCTGTCCA | |
| | GTCAGCGTGGAGAAGATCGTGAACA | |
| | TCTTCAACGGCACCTCTTGCCCAAG | |
| | TCTGGGCGGGAAGCCCAAACTGTTC | |
| | TTTATTCAGGCCTGTGGAGGCGAGC | |
| | AGAAAGATCACGGCTTCGAAGTGGC | |
| | TAGCACCTCCCCCGAGGACGAATCA | |
| | CCTGGAAGCAACCCTGAGCCAGATG | |
| | CAACCCCCTTCCAGGAAGGCCTGAG | |
| | GACATTTGACCAGCTGGATGCCATC | |
| | TCAAGCCTGCCCACACCTTCTGACA | |
| | TTTTCGTCTCTTACAGTACTTTCCCT | |
| | GGATTTGTGAGCTGGCGCGATCCAA | |
| | AGTCAGGCAGCTGGTACGTGGAGAC | |
| | ACTGGACGATATCTTTGAGCAGTGG | |
| | GCCCATTCTGAAGACCTGCAGAGTC | |
| | TGCTGCTGCGAGTGGCCAATGCTGT | |
| | CTCTGTGAAGGGGATCTACAAACAG | |
| | ATGCCAGGATGCTTCAACTTTCTGA | |
| | GAAAGAAACTGTTCTTTAAGACCTCC | |
| | GCATCTAGGGCC-(T2A)- | |
| | CCGCGGGAAGGCCGAGGGAGCCTG | |
| | CTGACATGTGGCGATGTGGAGGAAA | |
| | ACCCAGGACCA | |
| Fv-iCASP9 S144A-T2A | SEQ ID NO: 48 | SEQ ID NO: 49 |
| | (Fv-L)- | (Fv-L)- |
| | GTCGACGGATTTGGTGATGTCGGTG | VDGFGDVGALEaLRGNADLAYILSM |
| | CTCTTGAGgcTTTGAGGGGAAATGCA | EPCGHCLIINNVNFCRESGLRTRTG |
| | GATTTGGCTTACATCCTGAGCATGG | SNIDCEKLRRRFSSLHFMVEVKGDL |
| | AGCCCTGTGGCCACTGCCTCATTAT | TAKKMVLALLELARQDHGALDCCV |
| | CAACAATGTGAACTTCTGCCGTGAG | VVILSHGCQASHLQFPGAVYGTDG |
| | TCCGGGCTCCGCACCCGCACTGGC | CPVSVEKIVNIFNGTSCPSLGGKPK |
| | TCCAACATCGACTGTGAGAAGTTGC | LFFIQACGGEQKDHGFEVASTSPE |
| | GGCGTCGCTTCTCCTCGCTGCATTT | DESPGSNPEPDATPFQEGLRTFDQ |
| | CATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S144D-T2A | SEQ ID NO: 50 | SEQ ID NO: 51 |
| | (Fv-L)- | (Fv-L)- |
| | GTCGACGGATTTGGTGATGTCGGTG | VDGFGDVGALEdLRGNADLAYILSM |
| | CTCTTGAGgacTTGAGGGGAAATGCA | EPCGHCLIINNVNFCRESGLRTRTG |
| | GATTTGGCTTACATCCTGAGCATGG | SNIDCEKLRRRFSSLHFMVEVKGDL |
| | AGCCCTGTGGCCACTGCCTCATTAT | TAKKMVLALLELARQDHGALDCCV |
| | CAACAATGTGAACTTCTGCCGTGAG | VVILSHGCQASHLQFPGAVYGTDG |
| | TCCGGGCTCCGCACCCGCACTGGC | CPVSVEKIVNIFNGTSCPSLGGKPK |
| | TCCAACATCGACTGTGAGAAGTTGC | LFFIQACGGEQKDHGFEVASTSPE |
| | GGCGTCGCTTCTCCTCGCTGCATTT | DESPGSNPEPDATPFQEGLRTFDQ |
| | CATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S183A-T2A | SEQ ID NO: 52 | SEQ ID NO: 53 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | aNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CgCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S196A-T2A | SEQ ID NO: 54 | SEQ ID NO: 55 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSaLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCgCGCTGCATT | DESPGSNPEPDATPFQEGLRTFDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S196D-T2A | SEQ ID NO: 56 | SEQ ID NO: 57 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSdLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCgacCTGCATTT | DESPGSNPEPDATPFQEGLRTFDQ |
| | CATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 C285A-T2A | SEQ ID NO: 58 | SEQ ID NO: 59 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQAaGGEQKDHGFEVASTSPED |
| | CGGCGTCGCTTCTCCTCGCTGCATT | ESPGSNPEPDATPFQEGLRTFDQL |
| | TCATGGTGGAGGTGAAGGGCGACCT | DAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCgcgGGTGGGG | |
| | AGCAGAAAGACCATGGGTTTGAGGT | |
| | GGCCTCCACTTCCCCTGAAGACGAG | |
| | TCCCCTGGCAGTAACCCCGAGCCAG | |
| | ATGCCACCCCGTTCCAGGAAGGTTT | |
| | GAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 A316G-T2A | SEQ ID NO: 60 | SEQ ID NO: 61 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDgTPFQEGLRTFDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGgCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 T317A-T2A | SEQ ID NO: 62 | SEQ ID NO: 63 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | aPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA- |
| | GGCTGTCTACGGCACAGATGGATGC | (T2A) |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCgCCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 T317C-T2A | SEQ ID NO: 64 | SEQ ID NO: 65 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | cPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA- |
| | GGCTGTCTACGGCACAGATGGATGC | (T2A) |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCtgCCCGTTCCAGGAAGGTTT | |
| | GAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 T317S-T2A | SEQ ID NO: 66 | SEQ ID NO: 67 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | sPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA- |
| | GGCTGTCTACGGCACAGATGGATGC | (T2A) |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCtCCCCGTTCCAGGAAGGTTT | |
| | GAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 F326K-T2A | SEQ ID NO: 68 | SEQ ID NO: 69 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTkDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCaagGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC | |
| Fv-iCASP9 D327K-T2A | SEQ ID NO: 70 | SEQ ID NO: 71 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFkQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCaAgCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 D327R-T2A | SEQ ID NO: 72 | SEQ ID NO: 73 |
| | GTCGACGGATTTGGTGATGTCGGTG | (Fv-L)- |
| | CTCTTGAGAGTTTGAGGGGAAATGC | VDGFGDVGALESLRGNADLAYILSM |
| | AGATTTGGCTTACATCCTGAGCATG | EPCGHCLIINNVNFCRESGLRTRTG |
| | GAGCCCTGTGGCCACTGCCTCATTA | SNIDCEKLRRRFSSLHFMVEVKGDL |
| | TCAACAATGTGAACTTCTGCCGTGA | TAKKMVLALLELARQDHGALDCCV |
| | GTCCGGGCTCCGCACCCGCACTGG | VVILSHGCQASHLQFPGAVYGTDG |
| | CTCCAACATCGACTGTGAGAAGTTG | CPVSVEKIVNIFNGTSCPSLGGKPK |
| | CGGCGTCGCTTCTCCTCGCTGCATT | LFFIQACGGEQKDHGFEVASTSPE |
| | TCATGGTGGAGGTGAAGGGCGACCT | DESPGSNPEPDATPFQEGLRTFrQL |
| | GACTGCCAAGAAAATGGTGCTGGCT | DAISSLPTPSDIFVSYSTFPGFVSW |
| | TTGCTGGAGCTGGCGCGGCAGGAC | RDPKSGSVVYVETLDDIFEQWAHSE |
| | CACGGTGCTCTGGACTGCTGCGTGG | DLQSLLLRVANAVSVKGIYKQMPGC |
| | TGGTCATTCTCTCTCACGGCTGTCA | FNFLRKKLFFKTSASRA-(T2A) |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCaggCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 D327G-T2A | SEQ ID NO: 74 | SEQ ID NO: 75 |
| | GTCGACGGATTTGGTGATGTCGGTG | (Fv-L)- |
| | CTCTTGAGAGTTTGAGGGGAAATGC | VDGFGDVGALESLRGNADLAYILSM |
| | AGATTTGGCTTACATCCTGAGCATG | EPCGHCLIINNVNFCRESGLRTRTG |
| | GAGCCCTGTGGCCACTGCCTCATTA | SNIDCEKLRRRFSSLHFMVEVKGDL |
| | TCAACAATGTGAACTTCTGCCGTGA | TAKKMVLALLELARQDHGALDCCV |
| | GTCCGGGCTCCGCACCCGCACTGG | VVILSHGCQASHLQFPGAVYGTDG |
| | CTCCAACATCGACTGTGAGAAGTTG | CPVSVEKIVNIFNGTSCPSLGGKPK |
| | CGGCGTCGCTTCTCCTCGCTGCATT | LFFIQACGGEQKDHGFEVASTSPE |
| | TCATGGTGGAGGTGAAGGGCGACCT | DESPGSNPEPDATPFQEGLRTFgQ |
| | GACTGCCAAGAAAATGGTGCTGGCT | LDAISSLPTPSDIFVSYSTFPGFVSW |
| | TTGCTGGAGCTGGCGCGGCAGGAC | RDPKSGSVVYVETLDDIFEQWAHSE |
| | CACGGTGCTCTGGACTGCTGCGTGG | DLQSLLLRVANAVSVKGIYKQMPGC |
| | TGGTCATTCTCTCTCACGGCTGTCA | FNFLRKKLFFKTSASRA-(T2A) |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGgCCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 Q328K-T2A | SEQ ID NO: 76 | SEQ ID NO: 77 |
| | (Fv-L)- | VDGFGDVGALESLRGNADLAYILSM |
| | GTCGACGGATTTGGTGATGTCGGTG | EPCGHCLIINNVNFCRESGLRTRTG |
| | CTCTTGAGAGTTTGAGGGGAAATGC | SNIDCEKLRRRFSSLHFMVEVKGDL |
| | AGATTTGGCTTACATCCTGAGCATG | TAKKMVLALLELARQDHGALDCCV |
| | GAGCCCTGTGGCCACTGCCTCATTA | VVILSHGCQASHLQFPGAVYGTDG |
| | TCAACAATGTGAACTTCTGCCGTGA | CPVSVEKIVNIFNGTSCPSLGGKPK |
| | GTCCGGGCTCCGCACCCGCACTGG | LFFIQACGGEQKDHGFEVASTSPE |
| | CTCCAACATCGACTGTGAGAAGTTG | DESPGSNPEPDATPFQEGLRTFDkL |
| | CGGCGTCGCTTCTCCTCGCTGCATT | DAISSLPTPSDIFVSYSTFPGFVSW |
| | TCATGGTGGAGGTGAAGGGCGACCT | RDPKSGSVVYVETLDDIFEQWAHSE |
| | GACTGCCAAGAAAATGGTGCTGGCT | DLQSLLLRVANAVSVKGIYKQMPGC |
| | TTGCTGGAGCTGGCGCGGCAGGAC | FNFLRKKLFFKTSASRA-(T2A) |
| | CACGGTGCTCTGGACTGCTGCGTGG | |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACaAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 0328R-T2A | SEQ ID NO: 78 | SEQ ID NO: 79 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDrL |
| | TCATGGTGGAGGTGAAGGGCGACCT | DAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACagGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 L329K-T2A | SEQ ID NO: 80 | SEQ ID NO: 81 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | kDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGaaGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC | |
| Fv-iCASP9 L329E-T2A | SEQ ID NO: 82 | SEQ ID NO: 83 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | eDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGgaGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 L329G-T2A | SEQ ID NO: 84 | SEQ ID NO: 85 |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | gDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGggcGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC | |
| Fv-L-Caspase9 D330A- | SEQ ID NO: 86 | SEQ ID NO: 87 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLaAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-Caspase9 D330E- | SEQ ID NO: 88 | SEQ ID NO: 89 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLeAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-Caspase9 D330N- | SEQ ID NO: 90 | SEQ ID NO: 91 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLnAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-Caspase9 D330V- | SEQ ID NO: 92 | SEQ ID NO: 93 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLvAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-Caspase9 D330G- | SEQ ID NO: 94 | SEQ ID NO: 95 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLgAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-Caspase9 D330S- | SEQ ID NO: 96 | SEQ ID NO: 97 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLsAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 A331K-T2A | SEQ ID NO: 98 | SEQ ID NO: 99 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | LDklSSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACaa | |
| | gATATCTAGTTTGCCCACACCCAGTG | |
| | ACATCTTTGTGTCCTACTCTACTTTC | |
| | CCAGGTTTTGTTTCCTGGAGGGACC | |
| | CCAAGAGTGGCTCCTGGTACGTTGA | |
| | GACCCTGGACGACATCTTTGAGCAG | |
| | TGGGCTCACTCTGAAGACCTGCAGT | |
| | CCCTCCTGCTTAGGGTCGCTAATGC | |
| | TGTTTCGGTGAAAGGGATTTATAAAC | |
| | AGATGCCTGGTTGCTTTAATTTCCTC | |
| | CGGAAAAAACTTTTCTTTAAAACATC | |
| | AGCTAGCAGAGCC-(T2A) | |
| Fv-L-iCaspase9 F404Y- | SEQ ID NO: 100 | SEQ ID NO: 101 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCyNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTaTAATTTCCT | |
| | CCGGAAAAAACTTTTCTTTAAAACAT | |
| | CAGCTAGCAGAGCC-(T2A) | |
| Fv-L-ICASP9 F404W-T2A | SEQ ID NO: 102 | SEQ ID NO: 103 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCwNFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTggAATTTCCT | |
| | CCGGAAAAAACTTTTCTTTAAAACAT | |
| | CAGCTAGCAGAGCC-(T2A) | |
| Fv-L-iCaspase9 N405Q- | SEQ ID NO: 104 | SEQ ID NO: 105 |
| T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFqFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTcagTTCCT | |
| | CCGGAAAAAACTTTTCTTTAAAACAT | |
| | CAGCTAGCAGAGCC-(T2A) | |
| Fv-L-iCaspase9 N405Q | SEQ ID NO: 106 | SEQ ID NO: 107 |
| codon optimized-T2A | -(Fv-L)- | (Fv-L)- |
| | GTGGACGGGTTTGGAGATGTGGGA | VDGFGDVGALESLRGNADLAYILSM |
| | GCCCTGGAATCCCTGCGGGGCAAT | EPCGHCLIINNVNFCRESGLRTRTG |
| | GCCGATCTGGCTTACATCCTGTCTAT | SNIDCEKLRRRFSS |
| | GGAGCCTTGCGGCCACTGTCTGATC | LHFMVEVKGDLTAKKMVLALLELAR |
| | ATTAACAATGTGAACTTCTGCAGAGA | QDHGALDCCVVVILSHGCQASHLQ |
| | GAGCGGGCTGCGGACCAGAACAGG | FPGAVYGTDGC |
| | ATCCAATATTGACTGTGAAAAGCTGC | PVSVEKIVNIFNGTSCPSLGGKPKLF |
| | GGAGAAGGTTCTCTAGTCTGCACTT | FIQACGGEQKDHGFEVASTSPEDE |
| | TATGGTCGAGGTGAAAGGCGATCTG | SPGSNPEPDA |
| | ACCGCTAAGAAAATGGTGCTGGCCC | TPFQEGLRTFDQLDAISSLPTPSDIF |
| | TGCTGGAACTGGCTCGGCAGGACCA | VSYSTFPGFVSWRDPKSGSVVYVET |
| | TGGGGCACTGGATTGCTGCGTGGTC | LDDIFEQWAH |
| | GTGATCCTGAGTCACGGCTGCCAGG | SEDLQSLLLRVANAVSVKGIYKQMP |
| | CTTCACATCTGCAGTTCCCTGGGGC | GCFqFLRKKLFFKTSASRA-(T2A) |
| | AGTCTATGGAACTGACGGCTGTCCA | |
| | GTCAGCGTGGAGAAGATCGTGAACA | |
| | TCTTCAACGGCACCTCTTGCCCAAG | |
| | TCTGGGCGGGAAGCCCAAACTGTTC | |
| | TTTATTCAGGCCTGTGGAGGCGAGC | |
| | AGAAAGATCACGGCTTCGAAGTGGC | |
| | TAGCACCTCCCCCGAGGACGAATCA | |
| | CCTGGAAGCAACCCTGAGCCAGATG | |
| | CAACCCCCTTCCAGGAAGGCCTGAG | |
| | GACATTTGACCAGCTGGATGCCATC | |
| | TCAAGCCTGCCCACACCTTCTGACA | |
| | TTTTCGTCTCTTACAGTACTTTCCCT | |
| | GGATTTGTGAGCTGGCGCGATCCAA | |
| | AGTCAGGCAGCTGGTACGTGGAGAC | |
| | ACTGGACGATATCTTTGAGCAGTGG | |
| | GCCCATTCTGAAGACCTGCAGAGTC | |
| | TGCTGCTGCGAGTGGCCAATGCTGT | |
| | CTCTGTGAAGGGGATCTACAAACAG | |
| | ATGCCAGGATGCTTCcagTTTCTGAG | |
| | AAAGAAACTGTTCTTTAAGACCTCCG | |
| | CATCTAGGGCC-(T2A) | |
| Fv-iCASP9 F406L-T2A | SEQ ID NO: 108 | SEQ ID NO: 109 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNLLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATcTCCT | |
| | CCGGAAAAAACTTTTCTTTAAAACAT | |
| | CAGCTAGCAGAGCC-(T2A) | |
| Fv-iCASP9 F406T-T2A | SEQ ID NO: 110 | SEQ ID NO: 111 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDATPFQEGLRTFDQ |
| | TCATGGTGGAGGTGAAGGGCGACCT | LDAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNtLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAAttcCCTC | |
| | CGGAAAAAACTTTTCTTTAAAACATC | |
| | AGCTAGCAGAGCC-(T2A) | |
| Fv-L-iCaspase9 S144A | SEQ ID NO: 112 | SEQ ID NO: 113 |
| N405Q-T2A codon | (Fv-L)- | (Fv-L)- |
| optimized | GTGGACGGGTTTGGAGATGTGGGA | VDGFGDVGALEaLRGNADLAYILSM |
| | GCCCTGGAAgCCCTGCGGGGCAATG | EPCGHCLIINNVNFCRESGLRTRTG |
| | CCGATCTGGCTTACATCCTGTCTATG | SNIDCEKLRRRFSS |
| | GAGCCTTGCGGCCACTGTCTGATCA | LHFMVEVKGDLTAKKMVLALLELAR |
| | TTAACAATGTGAACTTCTGCAGAGAG | QDHGALDCCVVVILSHGCQASHLQ |
| | AGCGGGCTGCGGACCAGAACAGGA | FPGAVYGTDGC |
| | TCCAATATTGACTGTGAAAAGCTGC | PVSVEKIVNIFNGTSCPSLGGKPKLF |
| | GGAGAAGGTTCTCTAGTCTGCACTT | FIQACGGEQKDHGFEVASTSPEDE |
| | TATGGTCGAGGTGAAAGGCGATCTG | SPGSNPEPDA |
| | ACCGCTAAGAAAATGGTGCTGGCCC | TPFQEGLRTFDQLDAISSLPTPSDIF |
| | TGCTGGAACTGGCTCGGCAGGACCA | VSYSTFPGFVSWRDPKSGSVVYVET |
| | TGGGGCACTGGATTGCTGCGTGGTC | LDDIFEQWAH |
| | GTGATCCTGAGTCACGGCTGCCAGG | SEDLQSLLLRVANAVSVKGIYKQMP |
| | CTTCACATCTGCAGTTCCCTGGGGC | GCFqFLRKKLFFKTSASRA-(T2A) |
| | AGTCTATGGAACTGACGGCTGTCCA | |
| | GTCAGCGTGGAGAAGATCGTGAACA | |
| | TCTTCAACGGCACCTCTTGCCCAAG | |
| | TCTGGGCGGGAAGCCCAAACTGTTC | |
| | TTTATTCAGGCCTGTGGAGGCGAGC | |
| | AGAAAGATCACGGCTTCGAAGTGGC | |
| | TAGCACCTCCCCCGAGGACGAATCA | |
| | CCTGGAAGCAACCCTGAGCCAGATG | |
| | CAACCCCCTTCCAGGAAGGCCTGAG | |
| | GACATTTGACCAGCTGGATGCCATC | |
| | TCAAGCCTGCCCACACCTTCTGACA | |
| | TTTTCGTCTCTTACAGTACTTTCCCT | |
| | GGATTTGTGAGCTGGCGCGATCCAA | |
| | AGTCAGGCAGCTGGTACGTGGAGAC | |
| | ACTGGACGATATCTTTGAGCAGTGG | |
| | GCCCATTCTGAAGACCTGCAGAGTC | |
| | TGCTGCTGCGAGTGGCCAATGCTGT | |
| | CTCTGTGAAGGGGATCTACAAACAG | |
| | ATGCCAGGATGCTTCcagTTTCTGAG | |
| | AAAGAAACTGTTCTTTAAGACCTCCG | |
| | CATCTAGGGCC-(T2A) | |
| Fv-iCASP9 S144A | SEQ ID NO: 114 | SEQ ID NO: 115 |
| D330A-T2A | (Fv-L)- | (Fv-L)- |
| | GTCGACGGATTTGGTGATGTCGGTG | VDGFGDVGALEaLRGNADLAYILSM |
| | CTCTTGAGgcTTTGAGGGGAAATGCA | EPCGHCLIINNVNFCRESGLRTRTG |
| | GATTTGGCTTACATCCTGAGCATGG | SNIDCEKLRRRFSSLHFMVEVKGDL |
| | AGCCCTGTGGCCACTGCCTCATTAT | TAKKMVLALLELARQDHGALDCCV |
| | CAACAATGTGAACTTCTGCCGTGAG | VVILSHGCQASHLQFPGAVYGTDG |
| | TCCGGGCTCCGCACCCGCACTGGC | CPVSVEKIVNIFNGTSCPSLGGKPK |
| | TCCAACATCGACTGTGAGAAGTTGC | LFFIQACGGEQKDHGFEVASTSPE |
| | GGCGTCGCTTCTCCTCGCTGCATTT | DESPGSNPEPDATPFQEGLRTFDQ |
| | CATGGTGGAGGTGAAGGGCGACCT | LaAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S144D | SEQ ID NO: 116 | SEQ ID NO: 117 |
| D330A-T2A | (Fv-L)- | (Fv-L)- |
| | GTCGACGGATTTGGTGATGTCGGTG | VDGFGDVGALEdLRGNADLAYILSM |
| | CTCTTGAGgacTTGAGGGGAAATGCA | EPCGHCLIINNVNFCRESGLRTRTG |
| | GATTTGGCTTACATCCTGAGCATGG | SNIDCEKLRRRFSSLHFMVEVKGDL |
| | AGCCCTGTGGCCACTGCCTCATTAT | TAKKMVLALLELARQDHGALDCCV |
| | CAACAATGTGAACTTCTGCCGTGAG | VVILSHGCQASHLQFPGAVYGTDG |
| | TCCGGGCTCCGCACCCGCACTGGC | CPVSVEKIVNIFNGTSCPSLGGKPK |
| | TCCAACATCGACTGTGAGAAGTTGC | LFFIQACGGEQKDHGFEVASTSPE |
| | GGCGTCGCTTCTCCTCGCTGCATTT | DESPGSNPEPDATPFQEGLRTFDQ |
| | CATGGTGGAGGTGAAGGGCGACCT | LaAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S196A | SEQ ID NO: 118 | SEQ ID NO: 119 |
| D330A-T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSaLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCgCGCTGCATT | DESPGSNPEPDATPFQEGLRTFDQ |
| | TCATGGTGGAGGTGAAGGGCGACCT | LaAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 S196D | SEQ ID NO: 120 | SEQ ID NO: 121 |
| D330A-T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSdLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCgacCTGCATTT | DESPGSNPEPDATPFQEGLRTFDQ |
| | CATGGTGGAGGTGAAGGGCGACCT | LaAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-L-iCaspase9 T317S | SEQ ID NO: 122 | SEQ ID NO: 123 |
| N405Q-T2A codon | (Fv-L)- | (Fv-L)- |
| optimized | GTGGACGGGTTTGGAGATGTGGGA | VDGFGDVGALESLRGNADLAYILSM |
| | GCCCTGGAATCCCTGCGGGGCAAT | EPCGHCLIINNVNFCRESGLRTRTG |
| | GCCGATCTGGCTTACATCCTGTCTAT | SNIDCEKLRRRFSS |
| | GGAGCCTTGCGGCCACTGTCTGATC | LHFMVEVKGDLTAKKMVLALLELAR |
| | ATTAACAATGTGAACTTCTGCAGAGA | QDHGALDCCVVVILSHGCQASHLQ |
| | GAGCGGGCTGCGGACCAGAACAGG | FPGAVYGTDGC |
| | ATCCAATATTGACTGTGAAAAGCTGC | PVSVEKIVNIFNGTSCPSLGGKPKLF |
| | GGAGAAGGTTCTCTAGTCTGCACTT | FIQACGGEQKDHGFEVASTSPEDE |
| | TATGGTCGAGGTGAAAGGCGATCTG | SPGSNPEPDA |
| | ACCGCTAAGAAAATGGTGCTGGCCC | sPFQEGLRTFDQLDAISSLPTPSDIF |
| | TGCTGGAACTGGCTCGGCAGGACCA | VSYSTFPGFVSWRDPKSGSVVYVET |
| | TGGGGCACTGGATTGCTGCGTGGTC | LDDIFEQWAH |
| | GTGATCCTGAGTCACGGCTGCCAGG | SEDLQSLLLRVANAVSVKGIYKQMP |
| | CTTCACATCTGCAGTTCCCTGGGGC | GCFqFLRKKLFFKTSASRA-(T2A) |
| | AGTCTATGGAACTGACGGCTGTCCA | |
| | GTCAGCGTGGAGAAGATCGTGAACA | |
| | TCTTCAACGGCACCTCTTGCCCAAG | |
| | TCTGGGCGGGAAGCCCAAACTGTTC | |
| | TTTATTCAGGCCTGTGGAGGCGAGC | |
| | AGAAAGATCACGGCTTCGAAGTGGC | |
| | TAGCACCTCCCCCGAGGACGAATCA | |
| | CCTGGAAGCAACCCTGAGCCAGATG | |
| | CAAgCCCCTTCCAGGAAGGCCTGAG | |
| | GACATTTGACCAGCTGGATGCCATC | |
| | TCAAGCCTGCCCACACCTTCTGACA | |
| | TTTTCGTCTCTTACAGTACTTTCCCT | |
| | GGATTTGTGAGCTGGCGCGATCCAA | |
| | AGTCAGGCAGCTGGTACGTGGAGAC | |
| | ACTGGACGATATCTTTGAGCAGTGG | |
| | GCCCATTCTGAAGACCTGCAGAGTC | |
| | TGCTGCTGCGAGTGGCCAATGCTGT | |
| | CTCTGTGAAGGGGATCTACAAACAG | |
| | ATGCCAGGATGCTTCcagTTTCTGAG | |
| | AAAGAAACTGTTCTTTAAGACCTCCG | |
| | CATCTAGGGCC-(T2A) | |
| Fv-L-Caspase9 D330A | SEQ ID NO: 124 | SEQ ID NO: 125 |
| N405Q-T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLaAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | GCFqFLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGcCGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTcagTTTCCT | |
| | CCGGAAAAAACTTTTCTTTAAAACAT | |
| | CAGCTAGCAGAGCC-(T2A) | |
| Fv-iCASP9 | SEQ ID NO: 126 | SEQ ID NO: 127 |
| ATPF316AVPI-T2A | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSSLHFMVEVKGDL |
| | GAGCCCTGTGGCCACTGCCTCATTA | TAKKMVLALLELARQDHGALDCCV |
| | TCAACAATGTGAACTTCTGCCGTGA | VVILSHGCQASHLQFPGAVYGTDG |
| | GTCCGGGCTCCGCACCCGCACTGG | CPVSVEKIVNIFNGTSCPSLGGKPK |
| | CTCCAACATCGACTGTGAGAAGTTG | LFFIQACGGEQKDHGFEVASTSPE |
| | CGGCGTCGCTTCTCCTCGCTGCATT | DESPGSNPEPDAvPiQEGLRTFDQL |
| | TCATGGTGGAGGTGAAGGGCGACCT | DAISSLPTPSDIFVSYSTFPGFVSW |
| | GACTGCCAAGAAAATGGTGCTGGCT | RDPKSGSVVYVETLDDIFEQWAHSE |
| | TTGCTGGAGCTGGCGCGGCAGGAC | DLQSLLLRVANAVSVKGIYKQMPGC |
| | CACGGTGCTCTGGACTGCTGCGTGG | FNFLRKKLFFKTSASRA-(T2A) |
| | TGGTCATTCTCTCTCACGGCTGTCA | |
| | GGCCAGCCACCTGCAGTTCCCAGG | |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCgtgCCcaTCCAGGAAGGTTT | |
| | GAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCTGGTTGCTTTAATTTCCT | |
| | CCGGAAAAAACTTTTCTTTAAAACAT | |
| | CAGCTAGCAGAGCC-(T2A) | |
| Fv-iCASP9 isaqt-T2A | SEQ ID NO: 128 | SEQ ID NO: 129 |
| | (Fv-L)- | (Fv-L)- |
| | GTCGACGGATTTGGTGATGTCGGTG | VDGFGDVGALESLRGNADLAYILSM |
| | CTCTTGAGAGTTTGAGGGGAAATGC | EPCGHCLIINNVNFCRESGLRTRTG |
| | AGATTTGGCTTACATCCTGAGCATG | SNIDCEKLRRRFSS |
| | GAGCCCTGTGGCCACTGCCTCATTA | LHFMVEVKGDLTAKKMVLALLELAR |
| | TCAACAATGTGAACTTCTGCCGTGA | QDHGALDCCVVVILSHGCQASHLQ |
| | GTCCGGGCTCCGCACCCGCACTGG | FPGAVYGTDGC |
| | CTCCAACATCGACTGTGAGAAGTTG | PVSVEKIVNIFNGTSCPSLGGKPKLF |
| | CGGCGTCGCTTCTCCTCGCTGCATT | FIQACGGEQKDHGFEVASTSPEDE |
| | TCATGGTGGAGGTGAAGGGCGACCT | SPGSNPEPDA |
| | GACTGCCAAGAAAATGGTGCTGGCT | TPFQEGLRTFDQLDAISSLPTPSDIF |
| | TTGCTGGAGCTGGCGCGGCAGGAC | VSYSTFPGFVSWRDPKSGSVVYVET |
| | CACGGTGCTCTGGACTGCTGCGTGG | LDDIFEQWAH |
| | TGGTCATTCTCTCTCACGGCTGTCA | SEDLQSLLLRVANAVSVKGIYKQMP |
| | GGCCAGCCACCTGCAGTTCCCAGG | isaqtLRKKLFFKTSASRA-(T2A) |
| | GGCTGTCTACGGCACAGATGGATGC | |
| | CCTGTGTCGGTCGAGAAGATTGTGA | |
| | ACATCTTCAATGGGACCAGCTGCCC | |
| | CAGCCTGGGAGGGAAGCCCAAGCT | |
| | CTTTTTCATCCAGGCCTGTGGTGGG | |
| | GAGCAGAAAGACCATGGGTTTGAGG | |
| | TGGCCTCCACTTCCCCTGAAGACGA | |
| | GTCCCCTGGCAGTAACCCCGAGCCA | |
| | GATGCCACCCCGTTCCAGGAAGGTT | |
| | TGAGGACCTTCGACCAGCTGGACGC | |
| | CATATCTAGTTTGCCCACACCCAGT | |
| | GACATCTTTGTGTCCTACTCTACTTT | |
| | CCCAGGTTTTGTTTCCTGGAGGGAC | |
| | CCCAAGAGTGGCTCCTGGTACGTTG | |
| | AGACCCTGGACGACATCTTTGAGCA | |
| | GTGGGCTCACTCTGAAGACCTGCAG | |
| | TCCCTCCTGCTTAGGGTCGCTAATG | |
| | CTGTTTCGGTGAAAGGGATTTATAAA | |
| | CAGATGCCgatatccgcacagacaCTCCG | |
| | GAAAAAACTTTTCTTTAAAACATCAG | |
| | CTAGCAGAGCC-(T2A) | |

Partial sequence of a plasmid insert coding for a polypeptide that encodes an inducible Caspase-9 polypeptide and a chimeric antigen receptor that binds to CD19, separated by a 2A linker, wherein the two Caspase-9 polypeptide and the chimeric antigen receptor are separated during translation. The example of a chimeric antigen receptor provided herein may be further modified by including costimulatory polypeptides such as, for example, but not limited to, CD28, 4-1BB and OX40. The inducible Caspase-9 polypeptide provided herein may be substituted by an inducible modified Caspase-9 polypeptide, such as, for example, those provided herein.

FKBPv36
SEQ ID NO: 130
ATGCTGGAGGGAGTGCAGGTGGAGACTATTAGCCCCGGAGATGGCAGAACATTCCCCAAA
AGAGGACAGACTTGCGTCGTGCATTATACTGGAATGCTGGAAGACGGCAAGAAGGTGGAC
AGCAGCCGGGACCGAAACAAGCCCTTCAAGTTCATGCTGGGGAAGCAGGAAGTGATCCG
GGGCTGGGAGGAAGGAGTCGCACAGATGTCAGTGGGACAGAGGGCCAAACTGACTATTA
GCCCAGACTACGCTTATGGAGCAACCGGCCACCCCGGGATCATTCCCCCTCATGCTACAC
TGGTCTTCGATGTGGAGCTGCTGAAGCTGGAA
FKBPv36
SEQ ID NO: 131
MLEGVQVETISPGDGRTFPKRGQTCVVHYTGMLEDGKKVDSSRDRNKPFKFMLGKQEVIRGW
EEGVAQMSVGQRAKLTISPDYAYGATGHPGIIPPHATLVFDVELLKLE
Linker
SEQ ID NO: 132
AGCGGAGGAGGATCCGGA
Linker
SEQ ID NO: 133
SGGGSG
Caspase-9
SEQ ID NO: 134
GTGGACGGGTTTGGAGATGTGGGAGCCCTGGAATCCCTGCGGGGCAATGCCGATCTGGC
TTACATCCTGTCTATGGAGCCTTGCGGCCACTGTCTGATCATTAACAATGTGAACTTCTGCA
GAGAGAGCGGGCTGCGGACCAGAACAGGATCCAATATTGACTGTGAAAAGCTGCGGAGAA
GGTTCTCTAGTCTGCACTTTATGGTCGAGGTGAAAGGCGATCTGACCGCTAAGAAAATGGT
GCTGGCCCTGCTGGAACTGGCTCGGCAGGACCATGGGGCACTGGATTGCTGCGTGGTCG
TGATCCTGAGTCACGGCTGCCAGGCTTCACATCTGCAGTTCCCTGGGGCAGTCTATGGAA
CTGACGGCTGTCCAGTCAGCGTGGAGAAGATCGTGAACATCTTCAACGGCACCTCTTGCC
CAAGTCTGGGCGGGAAGCCCAAACTGTTCTTTATTCAGGCCTGTGGAGGCGAGCAGAAAG
ATCACGGCTTCGAAGTGGCTAGCACCTCCCCCGAGGACGAATCACCTGGAAGCAACCCTG
AGCCAGATGCAACCCCCTTCCAGGAAGGCCTGAGGACATTTGACCAGCTGGATGCCATCT
CAAGCCTGCCCACACCTTCTGACATTTTCGTCTCTTACAGTACTTTCCCTGGATTTGTGAGC
TGGCGCGATCCAAAGTCAGGCAGCTGGTACGTGGAGACACTGGACGATATCTTTGAGCAG
TGGGCCCATTCTGAAGACCTGCAGAGTCTGCTGCTGCGAGTGGCCAATGCTGTCTCTGTG
AAGGGGATCTACAAACAGATGCCAGGATGCTTCAACTTTCTGAGAAAGAAACTGTTCTTTAA
GACCTCCGCATCTAGGGCC
Caspase-9
SEQ ID NO: 135
VDGFGDVGALESLRGNADLAYILSMEPCGHCLIINNVNFCRESGLRTRTGSNIDCEKLRRRFSS
LHFMVEVKGDLTAKKMVLALLELARQDHGALDCCVVVILSHGCQASHLQFPGAVYGTDGCPVS
VEKIVNIFNGTSCPSLGGKPKLFFIQACGGEQKDHGFEVASTSPEDESPGSNPEPDATPFQEGL
RTFDQLDAISSLPTPSDIFVSYSTFPGFVSWRDPKSGSVVYVETLDDIFEQWAHSEDLQSLLLRV
ANAVSVKGIYKQMPGCFNFLRKKLFFKTSASRA
Linker
SEQ ID NO: 136
CCGCGG
Linker
SEQ ID NO: 137
PR
T2A
SEQ ID NO: 138
GAAGGCCGAGGGAGCCTGCTGACATGTGGCGATGTGGAGGAAAACCCAGGACCA
T2A
SEQ ID NO: 139
EGRGSLLTCGDVEENPGP
Linker
SEQ ID NO: 140
CCATGG
Linker
SEQ ID NO: 141
PW
Signal peptide
SEQ ID NO: 142
ATGGAGTTTGGACTTTCTTGGTTGTTTTTGGTGGCAATTCTGAAGGGTGTCCAGTGTAGCA
GG
Signal peptide
SEQ ID NO: 143
MEFGLSWLFLVAILKGVQCSR
FMC63 variable light chain (anti-CD19)
SEQ ID NO: 144
GACATCCAGATGACACAGACTACATCCTCCCTGTCTGCCTCTCTGGGAGACAGAGTCACCA
TCAGTTGCAGGGCAAGTCAGGACATTAGTAAATATTTAAATTGGTATCAGCAGAAACCAGAT
GGAACTGTTAAACTCCTGATCTACCATACATCAAGATTACACTCAGGAGTCCCATCAAGGTT
CAGTGGCAGTGGGTCTGGAACAGATTATTCTCTCACCATTAGCAACCTGGAGCAAGAAGAT
ATTGCCACTTACTTTTGCCAACAGGGTAATACGCTTCCGTACACGTTCGGAGGGGGGACTA
AGTTGGAAATAACA
FMC63 variable light chain (anti CD19)
SEQ ID NO: 145
DIQMTQTTSSLSASLGDRVTISCRASQDISKYLNVVYQQKPDGTVKLLIYHTSRLHSGVPSRFSG
SGSGTDYSLTISNLEQEDIATYFCQQGNTLPYTFGGGTKLEIT
Flexible linker
SEQ ID NO: 146
GGCGGAGGAAGCGGAGGTGGGGGC
Flexible linker
SEQ ID NO: 147
GGGSGGGG
FMC63 variable heavy chain (anti-CD19)
SEQ ID NO: 148
GAGGTGAAACTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGATTCGCCAGCCT
CCACGAAAGGGTCTGGAGTGGCTGGGAGTAATATGGGGTAGTGAAACCACATACTATAATT
CAGCTCTCAAATCCAGACTGACCATCATCAAGGACAACTCCAAGAGCCAAGTTTTCTTAAAA
ATGAACAGTCTGCAAACTGATGACACAGCCATTTACTACTGTGCCAAACATTATTACTACGG
TGGTAGCTATGCTATGGACTACTGGGGTCAAGGAACCTCAGTCACCGTCTCCTCA
FMC63 variable heavy chain (anti CD19)
SEQ ID NO: 149
EVKLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
Linker
SEQ ID NO: 150
GGATCC
Linker
SEQ ID NO: 151
GS
CD34 minimal epitope
SEQ ID NO: 152
GAACTTCCTACTCAGGGGACTTTCTCAAACGTTAGCACAAACGTAAGT
CD34 minimal epitope
SEQ ID NO: 153
ELPTQGTFSNVSTNVS
CD8 α stalk domain
SEQ ID NO: 154
CCCGCCCCAAGACCCCCCACACCTGCGCCGACCATTGCTTCTCAACCCCTGAGTTTGAGA
CCCGAGGCCTGCCGGCCAGCTGCCGGCGGGGCCGTGCATACAAGAGGACTCGATTTCGC
TTGCGAC
CD8 α stalk domain
SEQ ID NO: 155
PAPRPPTPAPTIASQPLSLRPEACRPAAGGAVHTRGLDFACD
CD8 α transmembrane domain
SEQ ID NO: 156
ATCTATATCTGGGCACCTCTCGCTGGCACCTGTGGAGTCCTTCTGCTCAGCCTGGTTATTA
CTCTGTACTGTAATCACCGGAATCGCCGCCGCGTTTGTAAGTGTCCCAGG
CD8 α transmembrane domain
SEQ ID NO: 157
IYIWAPLAGTCGVLLLSLVITLYCNHRNRRRVCKCPR
Linker
SEQ ID NO: 158
GTCGAC
Linker
SEQ ID NO: 159
VD
CD3 zeta
SEQ ID NO: 160
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta
SEQ ID NO: 161
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

Provided below is an example of a plasmid insert coding for a chimeric antigen receptor that binds to Her2/Neu. The chimeric antigen receptor may be further modified by including costimulatory polypeptides such as, for example, but not limited to, CD28, OX40, and 4-1BB.

Signal peptide
SEQ ID NO: 162
ATGGAGTTTGGACTTTCTTGGTTGTTTTTGGTGGCAATTCTGAAGGGTGTCCAGTGTAGCA
GG
Signal peptide
SEQ ID NO: 163
MEFGLSWLFLVAILKGVQCSR
FRP5 variable light chain (anti-Her2)
SEQ ID NO: 164
GACATCCAATTGACACAATCACACAAATTTCTCTCAACTTCTGTAGGAGACAGAGTGAGCAT
AACCTGCAAAGCATCCCAGGACGTGTACAATGCTGTGGCTTGGTACCAACAGAAGCCTGG
ACAATCCCCAAAATTGCTGATTTATTCTGCCTCTAGTAGGTACACTGGGGTACCTTCTCGGT
TTACGGGCTCTGGGTCCGGACCAGATTTCACGTTCACAATCAGTTCCGTTCAAGCTGAAGA
CCTCGCTGTTTATTTTTGCCAGCAGCACTTCCGAACCCCTTTTACTTTTGGCTCAGGCACTA
AGTTGGAAATCAAGGCTTTG
FRP5 variable light chain (anti-Her2)
SEQ ID NO: 165
DIQLTQSHKFLSTSVGDRVSITCKASQDVYNAVAVVYQQKPGQSPKWYSASSRYTGVPSRFT
GSGSGPDFTFTISSVQAEDLAVYFCQQHFRTPFTFGSGTKLEIKAL
Flexible linker
SEQ ID NO: 166
GGCGGAGGAAGCGGAGGTGGGGGC
Flexible linker
SEQ ID NO: 167
GGGSGGGG
FRP5 variable heavy chain (anti-Her2/Neu)
SEQ ID NO: 168
GAAGTCCAATTGCAACAGTCAGGCCCCGAATTGAAAAAGCCCGGCGAAACAGTGAAGATA
TCTTGTAAAGCCTCCGGTTACCCTTTTACGAACTATGGAATGAACTGGGTCAAACAAGCCC
CTGGACAGGGATTGAAGTGGATGGGATGGATCAATACATCAACAGGCGAGTCTACCTTCG
CAGATGATTTCAAAGGTCGCTTTGACTTCTCACTGGAGACCAGTGCAAATACCGCCTACCT
TCAGATTAACAATCTTAAAAGCGAGGATATGGCAACCTACTTTTGCGCAAGATGGGAAGTTT
ATCACGGGTACGTGCCATACTGGGGACAAGGAACGACAGTGACAGTTAGTAGC
FRP5 variable heavy chain (anti-Her2/Neu)
SEQ ID NO: 169
EVQLQQSGPELKKPGETVKISCKASGYPFTNYGMNVVVKQAPGQGLKVVMGWINTSTGESTFA
DDFKGRFDFSLETSANTAYLQINNLKSEDMATYFCARWEVYHGYVPYWGQGTTVTVSS
Linker
SEQ ID NO: 170
GGATCC
Linker
SEQ ID NO: 171
GS
CD34 minimal epitope
SEQ ID NO: 172
GAACTTCCTACTCAGGGGACTTTCTCAAACGTTAGCACAAACGTAAGT
CD34 minimal epitope
SEQ ID NO: 173
ELPTQGTFSNVSTNVS
CD8 alpha stalk
SEQ ID NO: 174
CCCGCCCCAAGACCCCCCACACCTGCGCCGACCATTGCTTCTCAACCCCTGAGTTTGAGA
CCCGAGGCCTGCCGGCCAGCTGCCGGCGGGGCCGTGCATACAAGAGGACTCGATTTCGC
TTGCGAC
CD8 alpha stalk
SEQ ID NO: 175
PAPRPPTPAPTIASQPLSLRPEACRPAAGGAVHTRGLDFACD
CD8 alpha transmembrane region
SEQ ID NO: 176
ATCTATATCTGGGCACCTCTCGCTGGCACCTGTGGAGTCCTTCTGCTCAGCCTGGTTATTA
CTCTGTACTGTAATCACCGGAATCGCCGCCGCGTTTGTAAGTGTCCCAGG
CD8 alpha transmembrane region
SEQ ID NO: 177
IYIWAPLAGTCGVLLLSLVITLYCNHRNRRRVCKCPR
Linker
SEQ ID NO: 178
Ctcgag
Linker
SEQ ID NO: 179
LE
CD3 zeta cytoplasmic domain
SEQ ID NO: 180
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta cytoplasmic domain
SEQ ID NO: 181
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
Additional sequences
CD28 nt
SEQ ID NO: 182
TTCTGGGTACTGGTTGTAGTCGGTGGCGTACTTGCTTGTTATTCTCTTCTTGTTACCGTAGC
CTTCATTATATTCTGGGTCCGATCAAAGCGCTCAAGACTCCTCCATTCCGATTATATGAACA
TGACACCTCGCCGACCTGGTCCTACACGCAAACATTATCAACCCTACGCACCCCCCCGAG
ACTTCGCTGCTTATCGATCC,
CD28 aa
SEQ ID NO: 183
FVVVLVVVGGVLACYSLLVTVAFIIFVVVRSKRSRLLHSDYMNMTPRRPGPTRKHYQPYAPPRDF
AAYRS,
OX40 nt
SEQ ID NO: 184
GTTGCCGCCATCCTGGGCCTGGGCCTGGTGCTGGGGCTGCTGGGCCCCCTGGCCATCCT
GCTGGCCCTGTACCTGCTCCGGGACCAGAGGCTGCCCCCCGATGCCCACAAGCCCCCTG
GGGGAGGCAGTTTCCGGACCCCCATCCAAGAGGAGCAGGCCGACGCCCACTCCACCCTG
GCCAAGATC,
OX40 aa
SEQ ID NO: 185
VAAILGLGLVLGLLGPLAILLALYLLRRDQRLPPDAHKPPGGGSFRTPIQEEQADAHSTLAKI,
4-1BB nt
SEQ ID NO: 186
AGTGTAGTTAAAAGAGGAAGAAAAAAGTTGCTGTATATATTTAAACAACCATTTATGAGACC
AGTGCAAACCACCCAAGAAGAAGACGGATGTTCATGCAGATTCCCAGAAGAAGAAGAAGG
AGGATGTGAATTG,
4-1BB aa
SEQ ID NO: 187
SVVKRGRKKLLYIFKQPFMRPVQTTQEEDGCSCRFPEEEEGGCEL,

### Expression of MyD88/CD40 Chimeric Antigen Receptors and Chimeric Stimulating Molecules

The following examples discuss the compositions and methods relating to MyD88/CD40 chimeric antigen receptors and chimeric stimulating molecules, as provided in this application. Also included are compositions and methods related to a Caspase-9-based safety switch, and its use in cells that express the MyD88/CD40 chimeric antigen receptors or chimeric stimulating molecules.

### Example 11: Design and Activity of MyD88/CD40 Chimeric Antigen Receptors

### Design of MC-CAR Constructs

Based on the activation data from inducible MyD88/CD40 experiments, the potential of MC signaling in a CAR molecule in place of conventional endodomains (e.g., CD28 and 4-1BB) was examined. MC (without AP1903-binding FKBPv36 regions) was subcloned into the PSCA.ζ to emulate the position of the CD28 endodomain. Retrovirus was generated for each of the three constructs, transduced human T cells and subsequently measured transduction efficiency demonstrating that PSCA.MC.ζ could be expressed. To confirm that T cells bearing each of these CAR constructs retained their ability to recognize PSCA⁺ tumor cells, 6-hour cytotoxicity assays were performed, which showed lysis of Capan-1 target cells. Therefore, the addition of MC into the cytoplasmic region of a CAR molecule does not affect CAR expression or the recognition of antigen on target cells.

MC costimulation enhances T cell killing, proliferation and survival in CAR-modified T cells As demonstrated in short-term cytotoxicity assays, each of the three CAR designs showed the capacity to recognize and lyse Capan-1 tumor cells. Cytolytic effector function in effector T cells is mediated by the release of pre-formed granzymes and perforin following tumor recognition, and activation through CD3ƒ is sufficient to induce this process without the need for costimulation. First generation CAR T cells (e.g., CARs constructed with only the CD3ζ cytoplasmic region) can lyse tumor cells; however, survival and proliferation is impaired due to lack of costimulation. Hence, the addition of CD28 or 4-1BB co-stimulating domains constructs has significantly improved the survival and proliferative capacity of CAR T cells.

To examine whether MC can similarly provide costimulating signals affecting survival and proliferation, coculture assays were performed with PSCA⁺ Capan-1 tumor cells under high tumor:T cell ratios (1:1, 1:5, 1:10 T cell to tumor cell). When T cell and tumor cell numbers were equal (1:1), there was efficient killing of Capan-1-GFP cells from all three constructs compared to non-transduced control T cells. However, when the CAR T cells were challenged with high numbers of tumor cells (1:10), there was a significant reduction of Capan-1-GFP tumor cells only when the CAR molecule contained either MC or CD28.

To further examine the mechanism of costimulation by these two CARs cell viability and proliferation was assayed. PSCA CARs containing MC or CD28 showed improved survival compared to non-transduced T cells and the CD3ζ only CAR, and T cell proliferation by PSCA.MC.ζ and PSCA.28.ζ was significantly enhanced. As other groups have shown that CARs that contain co-stimulating signaling regions produce IL-2, a key survival and growth molecule for T cells (4), ELISAs were performed on supernatants from CAR T cells challenged with Capan-1 tumor cells. Although PSCA.28.ζ produced high levels of IL-2, PSCA.MC.ζ signaling also produced significant levels of IL-2, which likely contributes to the observed T cell survival and expansion in these assays. Additionally, IL-6 production by CAR-modified T cells was examined, as IL-6 has been implicated as a key cytokine in the potency and efficacy of CAR-modified T cells (15). In contrast to IL-2, PSCA.MC.ζ produced higher levels of IL-6 compared to PSCA.28., consistent with the observations that iMC activation in primary T cells induces IL-6. Together, these data suggest that co-stimulation through MC produces similar effects to that of CD28, whereby following tumor cell recognition, CAR-modified T cells produce IL-2 and IL-6, which enhance T cell survival

Immunotherapy using CAR-modified T cells holds great promise for the treatment of a variety of malignancies. While CARs were first designed with a single signaling domain (e.g., CD3ζ, (16-19) clinical trials evaluating the feasibility of CAR immunotherapy showed limited clinical benefit. (1, 2, 20, 21) This has been primarily attributed to the incomplete activation of T cells following tumor recognition, which leads to limited persistence and expansion in vivo. (22) To address this deficiency, CARs have been engineered to include another stimulating domain, often derived from the cytoplasmic portion of T cell costimulating molecules including CD28, 4-1BB, OX40, ICOS and DAP10, (4, 23-30) which allow CART cells to receive appropriate costimulation upon engagement of the target antigen. Indeed, clinical trials conducted with anti-CD19 CARs bearing CD28 or 4-1BB signaling domains for the treatment of refractory acute lymphoblastic leukemia (ALL) have demonstrated impressive T cell persistence, expansion and serial tumor killing following adoptive transfer. (6-8)

CD28 costimulation provides a clear clinical advantage for the treatment of CD19⁺ lymphomas. Savoldo and colleagues conducted a CAR-T cell clinical trial comparing first (CD19.ζ) and second generation CARs (CD19.28.ζ) and found that CD28 enhanced T cell persistence and expansion following adoptive transfer.31 One of the principal functions of second generation CARs is the ability to produce IL-2 that supports T cell survival and growth through activation of the N FAT transcription factor by CD3ζ (signal 1), and NF-κB (signal 2) by CD28 or 4-1BB.32 This suggested other molecules that similarly activated NF-κB might be paired with the CD3ζ chain within a CAR molecule. Our approach has employed a T cell costimulating molecule that was originally developed as an adjuvant for a dendritic cell (DC) vaccine. (12,33) For full activation or licensing of DCs, TLR signaling is usually involved in the upregulation of the TNF family member, CD40, which interacts with CD40L on antigen-primed CD4⁺ T cells. Because iMC was a potent activator of NF-κB in DCs, transduction of T cells with CARs that incorporated MyD88 and CD40 might provide the required costimulation (signal 2) to T cells, and enhance their survival and proliferation.

A set of experiments was performed to examine whether MyD88, CD40 or both components were required for optimum T cell stimulation using the iMC molecule. Remarkably, it was found that neither MyD88 nor CD40 could sufficiently induce T cell activation, as measured by cytokine production (IL-2 and IL-6), but when combined as a single fusion protein, could induce potent T cell activation. A PSCA CAR incorporating MC was constructed and its function was subsequently compared against a first (PSCA.ζ) and second generation (PSCA.28.ζ) CAR. Here, it was found that MC enhanced survival and proliferation of CAR T cells to a comparable level as the CD28 endodomain, suggesting that costimulation was sufficient. While PSCA.MC.ζ CAR-transduced T cells produced lower levels of IL-2 than PSCA.28., the secreted levels were significantly higher than non-transduced T cells and T cells transduced with the PSCA.ζ CAR. On the other hand, PSCA.MC.ζ CAR-transduced T cells secreted significantly higher levels of IL-6, an important cytokine associated with T cell activation, than PSCA.28.ζ transduced T cells, indicating that MC conferred unique properties to CAR function that may translate to improved tumor cell killing in vivo. These experiments indicate that MC can activate NF-κB (signal 2) following antigen recognition by the extracellular CAR domain.

### Design and Functional Validation of MC-CAR.

Three PSCA CAR constructs were designed incorporating only CD3ζ, or with CD28 or MC endodomains. Transduction efficiency (percentage) was measured by anti-CAR-APC (recognizing the IgG1 CH₂CH₃ domain). C) Flow cytometry analysis demonstrating high transduction efficiency of T cells with PSCA.MC.ζ CAR. D) Analysis of specific lysis of PSCA⁺ Capan-1 tumor cells by CAR-modified T cells in a 6-hour LDH release assay at a ratio of 1:1 T cells to tumor cells.

MC-CAR modified T cells kill Capan-1 tumor cells in long-term coculture assays. Flow cytometric analysis of CAR-modified and non-transduced T cells cultured with Capan-1-GFP tumor cells after 7 days in culture at a 1:1 ratio. Quantitation of viable GFP⁺ cells by flow cytometry in coculture assays at a 1:1 and 1:10 T cell to tumor cell ratio.

MC and CD28 costimulation enhance T cell survival, proliferation and cytokine production. T cells isolated from 1:10 T cell to tumor cell coculture assays were assayed for cell viability and cell number to assess survival and proliferation in response to tumor cell exposure. Supernatants from coculture assays were subsequently measured for IL-2 and IL-6 production by ELISA.

Design of inducible costimulating molecules and effect on T cell activation. Four vectors were designed incorporating FKBPv36 AP1903-binding domains (Fv′.Fv) alone, or with MyD88, CD40 or the MyD88/CD40 fusion protein. Transduction efficiency of primary activated T cells using CD3⁺CD19⁺ flow cytometric analysis. Analysis of IFN-γ production of modified T cells following activation with and without 10 nM AP1903. Analysis of IL-6 production of modified T cells following activation with and without 10 nM AP1903.

Apart from survival and growth advantages, MC-induced costimulation may also provide additional functions to CAR-modified T cells. Medzhitov and colleagues recently demonstrated that MyD88 signaling was critical for both Th1 and Th17 responses and that it acted via IL-1 to render CD4⁺ T cells refractory to regulatory T cell (Treg)-driven inhibition (34). Experiments with iMC show that IL-1α and β are secreted following AP1903 activation. In addition, Martin et al demonstrated that CD40 signaling in CD8⁺ T cells via Ras, PI3K and protein kinase C, result in NF-κB-dependent induction of cytotoxic mediators granzyme and perforin that lyse CD4⁺CD25⁺ Treg cells (35). Thus, MyD88 and CD40 co-activation may render CAR-T cells resistant to the immunosuppressive effects of Treg cells, a function that could be critically important in the treatment of solid tumors and other types of cancers.

In summary, MC can be incorporated into a CAR molecule and primary T cells transduced with retrovirus can express PSCA.MC.ζ without overt toxicity or CAR stability issues. Further, MC appears to provide similar costimulation to that of CD28, where transduced T cells show improved survival, proliferation and tumor killing compared to T cells transduced with a first generation CAR.

### Example 12: References

The following references are cited in, or provide additional information that may be relevant, including, for example, in Example 11.
1. Till B G, Jensen M C, Wang J, et al: CD20-specific adoptive immunotherapy for lymphoma using a chimeric antigen receptor with both CD28 and 4-1BB domains: pilot clinical trial results. Blood 119:3940-50, 2012.
2. Pule M A, Savoldo B, Myers G D, et al: Virus-specific T cells engineered to coexpress tumor-specific receptors: persistence and antitumor activity in individuals with neuroblastoma. Nat Med 14:1264-70, 2008.
3. Kershaw M H, Westwood J A, Parker L L, et al: A phase 1 study on adoptive immunotherapy using gene-modified T cells for ovarian cancer. Clin Cancer Res 12:6106-15, 2006.
4. Carpenito C, Milone M C, Hassan R, et al: Control of large, established tumor xenografts with genetically retargeted human T cells containing CD28 and CD137 domains. Proc Natl Acad Sci USA 106:3360-5, 2009.
5. Song D G, Ye Q, Poussin M, et al: CD27 costimulation augments the survival and antitumor activity of redirected human T cells in vivo. Blood 119:696-706, 2012.
6. Kalos M, Levine B L, Porter D L, et al: T cells with chimeric antigen receptors have potent antitumor effects and can establish memory in patients with advanced leukemia. Sci Transl Med 3:95ra73, 2011.
7. Porter D L, Levine B L, Kalos M, et al: Chimeric antigen receptor-modified T cells in chronic lymphoid leukemia. N Engl J Med 365:725-33, 2011.
8. Brentjens R J, Davila M L, Riviere I, et al: CD19-targeted T cells rapidly induce molecular remissions in adults with chemotherapy-refractory acute lymphoblastic leukemia. Sci Transl Med 5:177ra38, 2013.
9. Pule M A, Straathof K C, Dotti G, et al: A chimeric T cell antigen receptor that augments cytokine release and supports clonal expansion of primary human T cells. Mol Ther 12:933-41, 2005.
10. Finney H M, Akbar A N, Lawson A D: Activation of resting human primary T cells with chimeric receptors: costimulation from CD28, inducible costimulator, CD134, and CD137 in series with signals from the TCR zeta chain. J Immunol 172:104-13, 2004.
11. Guedan S, Chen X, Madar A, et al: ICOS-based chimeric antigen receptors program bipolar TH17/TH1 cells. Blood, 2014.
12. Narayanan P, Lapteva N, Seethammagari M, et al: A composite MyD88/CD40 switch synergistically activates mouse and human dendritic cells for enhanced antitumor efficacy. J Clin Invest 121:1524-34, 2011.
13. Anurathapan U, Chan R C, Hindi H F, et al: Kinetics of tumor destruction by chimeric antigen receptor-modified T cells. Mol Ther 22:623-33, 2014.
14. Craddock J A, Lu A, Bear A, et al: Enhanced tumor trafficking of GD2 chimeric antigen receptor T cells by expression of the chemokine receptor CCR2b. J Immunother 33:780-8, 2010.
15. Lee D W, Gardner R, Porter D L, et al: Current concepts in the diagnosis and management of cytokine release syndrome. Blood 124:188-95, 2014.
16. Becker M L, Near R, Mudgett-Hunter M, et al: Expression of a hybrid immunoglobulin-T cell receptor protein in transgenic mice. Cell 58:911-21, 1989.
17. Goverman J, Gomez S M, Segesman K D, et al: Chimeric immunoglobulin-T cell receptor proteins form functional receptors: implications for T cell receptor complex formation and activation. Cell 60:929-39, 1990.
18. Gross G, Waks T, Eshhar Z: Expression of immunoglobulin-T-cell receptor chimeric molecules as functional receptors with antibody-type specificity. Proc Natl Acad Sci USA 86:10024-8, 1989.
19. Kuwana Y, Asakura Y, Utsunomiya N, et al: Expression of chimeric receptor composed of immunoglobulin-derived V regions and T-cell receptor-derived C regions. Biochem Biophys Res Commun 149:960-8, 1987.
20. Jensen M C, Popplewell L, Cooper L J, et al: Antitransgene rejection responses contribute to attenuated persistence of adoptively transferred CD20/CD19-specific chimeric antigen receptor redirected T cells in humans. Biol Blood Marrow Transplant 16:1245-56, 2010.
21. Park J R, Digiusto D L, Slovak M, et al: Adoptive transfer of chimeric antigen receptor re-directed cytolytic T lymphocyte clones in patients with neuroblastoma. Mol Ther 15:825-33, 2007.
22. Ramos C A, Dotti G: Chimeric antigen receptor (CAR)-engineered lymphocytes for cancer therapy. Expert Opin Biol Ther 11:855-73, 2011.
23. Finney H M, Lawson A D, Bebbington C R, et al: Chimeric receptors providing both primary and costimulatory signaling in T cells from a single gene product. J Immunol 161:2791-7, 1998.
24. Hombach A, Wieczarkowiecz A, Marquardt T, et al: Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule. J Immunol 167:6123-31, 2001.
25. Maher J, Brentjens R J, Gunset G, et al: Human T-lymphocyte cytotoxicity and proliferation directed by a single chimeric TCRzeta/CD28 receptor. Nat Biotechnol 20:70-5, 2002.
26. Imai C, Mihara K, Andreansky M, et al: Chimeric receptors with 4-1BB signaling capacity provoke potent cytotoxicity against acute lymphoblastic leukemia. Leukemia 18:676-84, 2004.
27. Wang J, Jensen M, Lin Y, et al: Optimizing adoptive polyclonal T cell immunotherapy of lymphomas, using a chimeric T cell receptor possessing CD28 and CD137 costimulatory domains. Hum Gene Ther 18:712-25, 2007.
28. Zhao Y, Wang Q J, Yang S, et al: A herceptin-based chimeric antigen receptor with modified signaling domains leads to enhanced survival of transduced T lymphocytes and antitumor activity. J Immunol 183:5563-74, 2009.
29. Milone M C, Fish J D, Carpenito C, et al: Chimeric receptors containing CD137 signal transduction domains mediate enhanced survival of T cells and increased antileukemic efficacy in vivo. Mol Ther 17:1453-64, 2009.
30. Yvon E, Del Vecchio M, Savoldo B, et al: Immunotherapy of metastatic melanoma using genetically engineered GD2-specific T cells. Clin Cancer Res 15:5852-60, 2009.
31. Savoldo B, Ramos C A, Liu E, et al: CD28 costimulation improves expansion and persistence of chimeric antigen receptor-modified T cells in lymphoma patients. J Clin Invest 121:1822-6, 2011.
32. Kalinski P, Hilkens C M, Wierenga E A, et al: T-cell priming by type-1 and type-2 polarized dendritic cells: the concept of a third signal. Immunol Today 20:561-7, 1999.
33. Kemnade J O, Seethammagari M, Narayanan P, et al: Off-the-shelf Adenoviral-mediated Immunotherapy via Bicistronic Expression of Tumor Antigen and iMyD88/CD40 Adjuvant. Mol Ther, 2012.
34. Schenten D, Nish S A, Yu S, et al: Signaling through the adaptor molecule MyD88 in CD4⁺ T cells is required to overcome suppression by regulatory T cells. Immunity 40:78-90, 2014.
35. Martin S, Pahari S, Sudan R, et al: CD40 signaling in CD8⁺CD40⁺ T cells turns on contra-T regulatory cell functions. J Immunol 184:5510-8, 2010

### Example 13: MC Costimulation Enhances Function and Proliferation of CD19 CARs

Experiments similar to those discussed herein, are provided, using an antigen recognition moiety that recognizes the CD19 antigen. It is understood that the vectors provided herein may be modified to construct a MyD88/CD40 CAR construct that targets CD19⁺ tumor cells, which also incorporates an inducible Caspase-9 safety switch.

To examine whether MC costimulation functioned in CARs targeting other antigens, T cells were modified with either CD19.ζ or with CD19.MC.ζ. The cytotoxicity, activation and survival against CD19⁺ Burkitt's lymphoma cell lines (Raji and Daudi) of the modified cells were assayed. In coculture assays, T cells transduced with either CAR showed killing of CD19⁺ Raji cells at an effector to target ratio as low as 1:1. However, analysis of cytokine production from co-culture assays showed that CD19.MC.ζ transduced T cells produced higher levels of IL-2 and IL-6 compared to CD19., which is consistent with the costimulatory effects observed with iMC and PSCA CARs containing the MC signaling domain. Further, T cells transduced with CD19.MC.ζ showed enhanced proliferation following activation by Raji tumor cells. These data support earlier experiments demonstrating that MC signaling in CAR molecules improves T cell activation, survival and proliferation following ligation to a target antigen expressed on tumor cells.

pBP0526-SFG.iCasp9wt.2A.CD19scFv.CD34e.CD8stm.MC.zeta
FKBPv36
SEQ ID NO: 321
ATGCTGGAGGGAGTGCAGGTGGAGACTATTAGCCCCGGAGATGGCAGAACATTCCCCAAA
AGAGGACAGACTTGCGTCGTGCATTATACTGGAATGCTGGAAGACGGCAAGAAGGTGGAC
AGCAGCCGGGACCGAAACAAGCCCTTCAAGTTCATGCTGGGGAAGCAGGAAGTGATCCG
GGGCTGGGAGGAAGGAGTCGCACAGATGTCAGTGGGACAGAGGGCCAAACTGACTATTA
GCCCAGACTACGCTTATGGAGCAACCGGCCACCCCGGGATCATTCCCCCTCATGCTACAC
TGGTCTTCGATGTGGAGCTGCTGAAGCTGGAA
FKBPv36
SEQ ID NO: 322
MLEGVQVETISPGDGRTFPKRGQTCVVHYTGMLEDGKKVDSSRDRNKPFKFMLGKQEVIRGW
EEGVAQMSVGQRAKLTISPDYAYGATGHPGIIPPHATLVFDVELLKLE
Linker
SEQ ID NO: 323
AGCGGAGGAGGATCCGGA
Linker
SEQ ID NO: 324
SGGGSG
Caspase-9
SEQ ID NO: 325
GTGGACGGGTTTGGAGATGTGGGAGCCCTGGAATCCCTGCGGGGCAATGCCGATCTGGC
TTACATCCTGTCTATGGAGCCTTGCGGCCACTGTCTGATCATTAACAATGTGAACTTCTGCA
GAGAGAGCGGGCTGCGGACCAGAACAGGATCCAATATTGACTGTGAAAAGCTGCGGAGAA
GGTTCTCTAGTCTGCACTTTATGGTCGAGGTGAAAGGCGATCTGACCGCTAAGAAAATGGT
GCTGGCCCTGCTGGAACTGGCTCGGCAGGACCATGGGGCACTGGATTGCTGCGTGGTCG
TGATCCTGAGTCACGGCTGCCAGGCTTCACATCTGCAGTTCCCTGGGGCAGTCTATGGAA
CTGACGGCTGTCCAGTCAGCGTGGAGAAGATCGTGAACATCTTCAACGGCACCTCTTGCC
CAAGTCTGGGCGGGAAGCCCAAACTGTTCTTTATTCAGGCCTGTGGAGGCGAGCAGAAAG
ATCACGGCTTCGAAGTGGCTAGCACCTCCCCCGAGGACGAATCACCTGGAAGCAACCCTG
AGCCAGATGCAACCCCCTTCCAGGAAGGCCTGAGGACATTTGACCAGCTGGATGCCATCT
CAAGCCTGCCCACACCTTCTGACATTTTCGTCTCTTACAGTACTTTCCCTGGATTTGTGAGC
TGGCGCGATCCAAAGTCAGGCAGCTGGTACGTGGAGACACTGGACGATATCTTTGAGCAG
TGGGCCCATTCTGAAGACCTGCAGAGTCTGCTGCTGCGAGTGGCCAATGCTGTCTCTGTG
AAGGGGATCTACAAACAGATGCCAGGATGCTTCAACTTTCTGAGAAAGAAACTGTTCTTTAA
GACCTCCGCATCTAGGGCC
Caspase-9
SEQ ID NO: 326
VDGFGDVGALESLRGNADLAYILSMEPCGHCLIINNVNFCRESGLRTRTGSNIDCEKLRRRFSS
LHFMVEVKGDLTAKKMVLALLELARQDHGALDCCVVVILSHGCQASHLQFPGAVYGTDGCPVS
VEKIVNIFNGTSCPSLGGKPKLFFIQACGGEQKDHGFEVASTSPEDESPGSNPEPDATPFQEGL
RTFDQLDAISSLPTPSDIFVSYSTFPGFVSWRDPKSGSVVYVETLDDIFEQWAHSEDLQSLLLRV
ANAVSVKGIYKQMPGCFNFLRKKLFFKTSASRA
Linker
SEQ ID NO: 327
CCGCGG
Linker
SEQ ID NO: 328
PR
T2A
SEQ ID NO: 329
GAAGGCCGAGGGAGCCTGCTGACATGTGGCGATGTGGAGGAAAACCCAGGACCA
T2A
SEQ ID NO: 330
EGRGSLLTCGDVEENPGP
Linker
SEQ ID NO: 331
CCATGG
Linker
SEQ ID NO: 332
PW
Signal peptide
SEQ ID NO: 333
ATGGAGTTTGGACTTTCTTGGTTGTTTTTGGTGGCAATTCTGAAGGGTGTCCAGTGTAGCA
GG
Signal peptide
SEQ ID NO: 334
MEFGLSWLFLVAILKGVQCSR
FMC63 variable light chain (anti-CD19)
SEQ ID NO: 335
GACATCCAGATGACACAGACTACATCCTCCCTGTCTGCCTCTCTGGGAGACAGAGTCACCA
TCAGTTGCAGGGCAAGTCAGGACATTAGTAAATATTTAAATTGGTATCAGCAGAAACCAGAT
GGAACTGTTAAACTCCTGATCTACCATACATCAAGATTACACTCAGGAGTCCCATCAAGGTT
CAGTGGCAGTGGGTCTGGAACAGATTATTCTCTCACCATTAGCAACCTGGAGCAAGAAGAT
ATTGCCACTTACTTTTGCCAACAGGGTAATACGCTTCCGTACACGTTCGGAGGGGGGACTA
AGTTGGAAATAACA
FMC63 variable light chain (anti CD19)
SEQ ID NO: 336
DIQMTQTTSSLSASLGDRVTISCRASQDISKYLNVVYQQKPDGTVKLLIYHTSRLHSGVPSRFSG
SGSGTDYSLTISNLEQEDIATYFCQQGNTLPYTFGGGTKLEIT
Flexible linker
SEQ ID NO: 337
GGCGGAGGAAGCGGAGGTGGGGGC
Flexible linker
SEQ ID NO: 338
GGGSGGGG
FMC63 variable heavy chain (anti-CD19)
SEQ ID NO: 339
GAGGTGAAACTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGATTCGCCAGCCT
CCACGAAAGGGTCTGGAGTGGCTGGGAGTAATATGGGGTAGTGAAACCACATACTATAATT
CAGCTCTCAAATCCAGACTGACCATCATCAAGGACAACTCCAAGAGCCAAGTTTTCTTAAAA
ATGAACAGTCTGCAAACTGATGACACAGCCATTTACTACTGTGCCAAACATTATTACTACGG
TGGTAGCTATGCTATGGACTACTGGGGTCAAGGAACCTCAGTCACCGTCTCCTCA
FMC63 variable heavy chain (anti CD19)
SEQ ID NO: 340
EVKLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
Linker
SEQ ID NO: 341
GGATCC
Linker
SEQ ID NO: 342
GS
CD34 minimal epitope
SEQ ID NO: 343
GAACTTCCTACTCAGGGGACTTTCTCAAACGTTAGCACAAACGTAAGT
CD34 minimal epitope
SEQ ID NO: 344
ELPTQGTFSNVSTNVS
CD8 α stalk domain
SEQ ID NO: 345
CCCGCCCCAAGACCCCCCACACCTGCGCCGACCATTGCTTCTCAACCCCTGAGTTTGAGA
CCCGAGGCCTGCCGGCCAGCTGCCGGCGGGGCCGTGCATACAAGAGGACTCGATTTCGC
TTGCGAC
CD8 α stalk domain
SEQ ID NO: 346
PAPRPPTPAPTIASQPLSLRPEACRPAAGGAVHTRGLDFACD
CD8 α transmembrane domain
SEQ ID NO: 347
ATCTATATCTGGGCACCTCTCGCTGGCACCTGTGGAGTCCTTCTGCTCAGCCTGGTTATTA
CTCTGTACTGTAATCACCGGAATCGCCGCCGCGTTTGTAAGTGTCCCAGG
CD8 α transmembrane domain
SEQ ID NO: 348
IYIWAPLAGTCGVLLLSLVITLYCNHRNRRRVCKCPR
Linker
SEQ ID NO: 349
GTCGAC
Linker
SEQ ID NO: 350
VD
Truncated MyD88 lacking the TIR domain
SEQ ID NO: 351
ATGGCCGCTGGGGGCCCAGGCGCCGGATCAGCTGCTCCCGTATCTTCTACTTCTTCTTTG
CCGCTGGCTGCTCTGAACATGCGCGTGAGAAGACGCCTCTCCCTGTTCCTTAACGTTCGC
ACACAAGTCGCTGCCGATTGGACCGCCCTTGCCGAAGAAATGGACTTTGAATACCTGGAA
ATTAGACAACTTGAAACACAGGCCGACCCCACTGGCAGACTCCTGGACGCATGGCAGGGA
AGACCTGGTGCAAGCGTTGGACGGCTCCTGGATCTCCTGACAAAACTGGGACGCGACGAC
GTACTGCTTGAACTCGGACCTAGCATTGAAGAAGACTGCCAAAAATATATCCTGAAACAAC
AACAAGAAGAAGCCGAAAAACCTCTCCAAGTCGCAGCAGTGGACTCATCAGTACCCCGAA
CAGCTGAGCTTGCTGGGATTACTACACTCGACGACCCACTCGGACATATGCCTGAAAGATT
CGACGCTTTCATTTGCTATTGCCCCTCTGACATA
Truncated MyD88 lacking the TIR domain
SEQ ID NO: 352
MAAGGPGAGSAAPVSSTSSLPLAALNMRVRRRLSLFLNVRTQVAADVVTALAEEMDFEYLEIRQ
LETQADPTGRLLDAWQGRPGASVGRLLDLLTKLGRDDVLLELGPSIEEDCQKYILKQQQEEAE
KPLQVAAVDSSVPRTAELAGITTLDDPLGHMPERFDAFICYCPSDI
CD40 without the extracellular domain
SEQ ID NO: 353
AAGAAAGTTGCAAAGAAACCCACAAATAAAGCCCCACACCCTAAACAGGAACCCCAAGAAA
TCAATTTCCCAGATGATCTCCCTGGATCTAATACTGCCGCCCCGGTCCAAGAAACCCTGCA
TGGTTGCCAGCCTGTCACCCAAGAGGACGGAAAAGAATCACGGATTAGCGTACAAGAGAG
ACAA
CD40 without the extra cellular domain
SEQ ID NO: 354
KKVAKKPTNKAPHPKQEPQEINFPDDLPGSNTAAPVQETLHGCQPVTQEDGKESRISVQERQ
CD3 zeta
SEQ ID NO: 355
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta
SEQ ID NO: 356
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

### Example 14: Cytokine Production of T Cells Co-Expressing a MyD88/CD40 Chimeric Antigen Receptor and Inducible Caspase-9 Polypeptide

Various chimeric antigen receptor constructs were created to compare cytokine production of transduced T cells after exposure to antigen. The chimeric antigen receptor constructs all had an antigen recognition region that bound to CD19. It is understood that the vectors provided herein may be modified to construct a CAR construct that also incorporates an inducible Caspase-9 safety switch. It is further understood that the CAR construct may further comprise an FRB domain.

### Example 15: An Example of a MyD88/CD40 CAR Construct for Targeting Her2⁺ Tumor Cells

It is understood that the vectors provided herein may be modified to construct a MyD88/CD40 CAR construct that targets Her2⁺ tumor cells, which also incorporates an inducible Caspase-9 safety switch. It is further understood that the CAR construct may further comprise an FRB domain.

SFG-Her2scFv.CD34e.CD8stm.MC.zeta sequence
Signal peptide
SEQ ID NO: 357
ATGGAGTTTGGACTTTCTTGGTTGTTTTTGGTGGCAATTCTGAAGGGTGTCCAGTGTAGCA
GG
Signal peptide
SEQ ID NO: 358
MEFGLSWLFLVAILKGVQCSR
FRP5 variable light chain (anti-Her2)
SEQ ID NO: 359
GACATCCAATTGACACAATCACACAAATTTCTCTCAACTTCTGTAGGAGACAGAGTGAGCAT
AACCTGCAAAGCATCCCAGGACGTGTACAATGCTGTGGCTTGGTACCAACAGAAGCCTGG
ACAATCCCCAAAATTGCTGATTTATTCTGCCTCTAGTAGGTACACTGGGGTACCTTCTCGGT
TTACGGGCTCTGGGTCCGGACCAGATTTCACGTTCACAATCAGTTCCGTTCAAGCTGAAGA
CCTCGCTGTTTATTTTTGCCAGCAGCACTTCCGAACCCCTTTTACTTTTGGCTCAGGCACTA
AGTTGGAAATCAAGGCTTTG
FRP5 variable light chain (anti-Her2)
SEQ ID NO: 360
DIQLTQSHKFLSTSVGDRVSITCKASQDVYNAVAVVYQQKPGQSPKWYSASSRYTGVPSRFT
GSGSGPDFTFTISSVQAEDLAVYFCQQHFRTPFTFGSGTKLEIKAL
Flexible linker
SEQ ID NO: 361
GGCGGAGGAAGCGGAGGTGGGGGC
Flexible linker
SEQ ID NO: 362
GGGSGGGG
FRP5 variable heavy chain (anti-Her2/Neu)
SEQ ID NO: 363
GAAGTCCAATTGCAACAGTCAGGCCCCGAATTGAAAAAGCCCGGCGAAACAGTGAAGATA
TCTTGTAAAGCCTCCGGTTACCCTTTTACGAACTATGGAATGAACTGGGTCAAACAAGCCC
CTGGACAGGGATTGAAGTGGATGGGATGGATCAATACATCAACAGGCGAGTCTACCTTCG
CAGATGATTTCAAAGGTCGCTTTGACTTCTCACTGGAGACCAGTGCAAATACCGCCTACCT
TCAGATTAACAATCTTAAAAGCGAGGATATGGCAACCTACTTTTGCGCAAGATGGGAAGTTT
ATCACGGGTACGTGCCATACTGGGGACAAGGAACGACAGTGACAGTTAGTAGC
FRP5 variable heavy chain (anti-Her2/Neu)
SEQ ID NO: 364
EVQLQQSGPELKKPGETVKISCKASGYPFTNYGMNVVVKQAPGQGLKVVMGWINTSTGESTFA
DDFKGRFDFSLETSANTAYLQINNLKSEDMATYFCARWEVYHGYVPYWGQGTTVTVSS
Linker
SEQ ID NO: 365
GGATCC
Linker
SEQ ID NO: 366
GS
CD34 minimal epitope
SEQ ID NO: 367
GAACTTCCTACTCAGGGGACTTTCTCAAACGTTAGCACAAACGTAAGT
CD34 minimal epitope
SEQ ID NO: 368
ELPTQGTFSNVSTNVS
CD8 alpha stalk
SEQ ID NO: 369
CCCGCCCCAAGACCCCCCACACCTGCGCCGACCATTGCTTCTCAACCCCTGAGTTTGAGA
CCCGAGGCCTGCCGGCCAGCTGCCGGCGGGGCCGTGCATACAAGAGGACTCGATTTCGC
TTGCGAC
CD8 alpha stalk
SEQ ID NO: 370
PAPRPPTPAPTIASQPLSLRPEACRPAAGGAVHTRGLDFACD
CD8 alpha transmembrane region
SEQ ID NO: 371
ATCTATATCTGGGCACCTCTCGCTGGCACCTGTGGAGTCCTTCTGCTCAGCCTGGTTATTA
CTCTGTACTGTAATCACCGGAATCGCCGCCGCGTTTGTAAGTGTCCCAGG
CD8 alpha transmembrane region
SEQ ID NO: 372
IYIWAPLAGTCGVLLLSLVITLYCNHRNRRRVCKCPR
Linker
SEQ ID NO: 373
Ctcgag
Linker
SEQ ID NO: 374
LE
Truncated MyD88
SEQ ID NO: 375
ATGGCCGCTGGGGGCCCAGGCGCCGGATCAGCTGCTCCCGTATCTTCTACTTCTTCTTTG
CCGCTGGCTGCTCTGAACATGCGCGTGAGAAGACGCCTCTCCCTGTTCCTTAACGTTCGC
ACACAAGTCGCTGCCGATTGGACCGCCCTTGCCGAAGAAATGGACTTTGAATACCTGGAA
ATTAGACAACTTGAAACACAGGCCGACCCCACTGGCAGACTCCTGGACGCATGGCAGGGA
AGACCTGGTGCAAGCGTTGGACGGCTCCTGGATCTCCTGACAAAACTGGGACGCGACGAC
GTACTGCTTGAACTCGGACCTAGCATTGAAGAAGACTGCCAAAAATATATCCTGAAACAAC
AACAAGAAGAAGCCGAAAAACCTCTCCAAGTCGCAGCAGTGGACTCATCAGTACCCCGAA
CAGCTGAGCTTGCTGGGATTACTACACTCGACGACCCACTCGGACATATGCCTGAAAGATT
CGACGCTTTCATTTGCTATTGCCCCTCTGACATA
Truncated MyD88
SEQ ID NO: 376
MAAGGPGAGSAAPVSSTSSLPLAALNMRVRRRLSLFLNVRTQVAADVVTALAEEMDFEYLEIRQ
LETQADPTGRLLDAWQGRPGASVGRLLDLLTKLGRDDVLLELGPSIEEDCQKYILKQQQEEAE
KPLQVAAVDSSVPRTAELAGITTLDDPLGHMPERFDAFICYCPSDI
CD40 cytoplasmic domain
SEQ ID NO: 377
AAGAAAGTTGCAAAGAAACCCACAAATAAAGCCCCACACCCTAAACAGGAACCCCAAGAAA
TCAATTTCCCAGATGATCTCCCTGGATCTAATACTGCCGCCCCGGTCCAAGAAACCCTGCA
TGGTTGCCAGCCTGTCACCCAAGAGGACGGAAAAGAATCACGGATTAGCGTACAAGAGAG
ACAA
CD40 cytoplasmic domain
SEQ ID NO: 378
KKVAKKPTNKAPHPKQEPQEINFPDDLPGSNTAAPVQETLHGCQPVTQEDGKESRISVQERQ
Linker
SEQ ID NO: 379
gcggccgcagtcgag
Linker
SEQ ID NO: 380
AAAVE
CD3 zeta cytoplasmic domain
SEQ ID NO: 381
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta cytoplasmic domain
SEQ ID NO: 382
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
Example 16: Additional Sequences
ΔCasp9(res. 135-416)
SEQ ID NO: 383
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L D A I S S L P T P S D I F V S
Y S T F P G F V S W R D P K S G S W Y V E T L D D I F E Q W A H S E
D L Q S L L L R V A N A V S V K G I Y K Q M P G C F N F L R K K L F F
K T S,
ΔCasp9(res. 135-416) D330A, nucleotide sequence
SEQ ID NO: 384
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGCCGCCATATCTAGTT
TGCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTAATTTCCTCCGGAAAAAACTTTTCTTTAAAACATCA,
ΔCasp9(res. 135-416) D330A, amino acid sequence
SEQ ID NO: 385
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L A A I S S L P T P S D I F V S
Y S T F P G F V S W R D P K S G S W Y V E T L D D I F E Q W A H S E
D L Q S L L L R V A N A V S V K G I Y K Q M P G C F N F L R K K L F F
K T S,
ΔCasp9(res. 135-416) N405Q nucleotide sequence
SEQ ID NO: 386
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGACGCCATATCTAGTTT
GCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTCAGTTCCTCCGGAAAAAACTTTTCTTTAAAACATCA,
ΔCasp9(res. 135-416) N405Q amino acid sequence
SEQ ID NO: 387
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L D A I S S L P T P S D I F V S
Y S T F P G F V S W R D P K S G S W Y V E T L D D I F E Q W A H S E
D L Q S L L L R V A N A V S V K G I Y K Q M P G C F Q F L R K K L F F
K T S,
ΔCasp9(res. 135-416) D330A N405Q nucleotide sequence
SEQ ID NO: 388
GGATTTGGTGATGTCGGTGCTCTTGAGAGTTTGAGGGGAAATGCAGATTTGGCTTACATCC
TGAGCATGGAGCCCTGTGGCCACTGCCTCATTATCAACAATGTGAACTTCTGCCGTGAGTC
CGGGCTCCGCACCCGCACTGGCTCCAACATCGACTGTGAGAAGTTGCGGCGTCGCTTCTC
CTCGCTGCATTTCATGGTGGAGGTGAAGGGCGACCTGACTGCCAAGAAAATGGTGCTGGC
TTTGCTGGAGCTGGCGCgGCAGGACCACGGTGCTCTGGACTGCTGCGTGGTGGTCATTCT
CTCTCACGGCTGTCAGGCCAGCCACCTGCAGTTCCCAGGGGCTGTCTACGGCACAGATG
GATGCCCTGTGTCGGTCGAGAAGATTGTGAACATCTTCAATGGGACCAGCTGCCCCAGCC
TGGGAGGGAAGCCCAAGCTCTTTTTCATCCAGGCCTGTGGTGGGGAGCAGAAAGACCATG
GGTTTGAGGTGGCCTCCACTTCCCCTGAAGACGAGTCCCCTGGCAGTAACCCCGAGCCAG
ATGCCACCCCGTTCCAGGAAGGTTTGAGGACCTTCGACCAGCTGGCCGCCATATCTAGTT
TGCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCT
CACTCTGAAGACCTGCAGTCCCTCCTGCTTAGGGTCGCTAATGCTGTTTCGGTGAAAGGG
ATTTATAAACAGATGCCTGGTTGCTTTCAGTTCCTCCGGAAAAAACTTTTCTTTAAAACATCA,
ΔCasp9(res. 135-416)D330A N405Q amino acid sequence
SEQ ID NO: 389
G F G D V G A L E S L R G N A D L A Y I L S M E P C G H C L I I N N V
N F C R E S G L R T R T G S N I D C E K L R R R F S S L H F M V E V K
G D L T A K K M V L A L L E L A R Q D H G A L D C C V V V I L S H G C
Q A S H L Q F P G A V Y G T D G C P V S V E K I V N I F N G T S C P S
L G G K P K L F F I Q A C G G E Q K D H G F E V A S T S P E D E S P G
S N P E P D A T P F Q E G L R T F D Q L A A I S S L P T P S D I F V S
Y S T F P G F V S W R D P K S G S W Y V E T L D D I F E Q W A H S E
D L Q S L L L R V A N A V S V K G I Y K Q M P G C F Q F L R K K L F F
K T S,
Caspase-9.co nucleotide sequence
SEQ ID NO: 390
GTGGACGGGTTTGGAGATGTGGGAGCCCTGGAATCCCTGCGGGGCAATGCCGATCTGGC
TTACATCCTGTCTATGGAGCCTTGCGGCCACTGTCTGATCATTAACAATGTGAACTTCTGCA
GAGAGAGCGGGCTGCGGACCAGAACAGGATCCAATATTGACTGTGAAAAGCTGCGGAGAA
GGTTCTCTAGTCTGCACTTTATGGTCGAGGTGAAAGGCGATCTGACCGCTAAGAAAATGGT
GCTGGCCCTGCTGGAACTGGCTCGGCAGGACCATGGGGCACTGGATTGCTGCGTGGTCG
TGATCCTGAGTCACGGCTGCCAGGCTTCACATCTGCAGTTCCCTGGGGCAGTCTATGGAA
CTGACGGCTGTCCAGTCAGCGTGGAGAAGATCGTGAACATCTTCAACGGCACCTCTTGCC
CAAGTCTGGGCGGGAAGCCCAAACTGTTCTTTATTCAGGCCTGTGGAGGCGAGCAGAAAG
ATCACGGCTTCGAAGTGGCTAGCACCTCCCCCGAGGACGAATCACCTGGAAGCAACCCTG
AGCCAGATGCAACCCCCTTCCAGGAAGGCCTGAGGACATTTGACCAGCTGGATGCCATCT
CAAGCCTGCCCACACCTTCTGACATTTTCGTCTCTTACAGTACTTTCCCTGGATTTGTGAGC
TGGCGCGATCCAAAGTCAGGCAGCTGGTACGTGGAGACACTGGACGATATCTTTGAGCAG
TGGGCCCATTCTGAAGACCTGCAGAGTCTGCTGCTGCGAGTGGCCAATGCTGTCTCTGTG
AAGGGGATCTACAAACAGATGCCAGGATGCTTCCAGTTTCTGAGAAAGAAACTGTTCTTTA
AGACCTCCGCATCTAGGGCC,
Caspase-9.co amino acid sequence
SEQ ID NO: 391
VDGFGDVGALESLRGNADLAYILSMEPCGHCLIINNVNFCRESGLRTRTGSNIDCEKLRRRFSS
LHFMVEVKGDLTAKKMVLALLELARQDHGALDCCVVVILSHGCQASHLQFPGAVYGTDGCPVS
VEKIVNIFNGTSCPSLGGKPKLFFIQACGGEQKDHGFEVASTSPEDESPGSNPEPDATPFQEGL
RTFDQLDAISSLPTPSDIFVSYSTFPGFVSWRDPKSGSVVYVETLDDIFEQWAHSEDLQSLLLRV
ANAVSVKGIYKQMPGCFQFLRKKLFFKTSASRA,
SEQ ID NO: 392: Caspase9 D330E nucleotide sequence
GTCGACGGATTTGGTGATGTCGGTGCTCTTGAGAGTTTGAGGGGAAATGCAGATTTGGCTT
ACATCCTGAGCATGGAGCCCTGTGGCCACTGCCTCATTATCAACAATGTGAACTTCTGCCG
TGAGTCCGGGCTCCGCACCCGCACTGGCTCCAACATCGACTGTGAGAAGTTGCGGCGTC
GCTTCTCCTCGCTGCATTTCATGGTGGAGGTGAAGGGCGACCTGACTGCCAAGAAAATGG
TGCTGGCTTTGCTGGAGCTGGCGCGGCAGGACCACGGTGCTCTGGACTGCTGCGTGGTG
GTCATTCTCTCTCACGGCTGTCAGGCCAGCCACCTGCAGTTCCCAGGGGCTGTCTACGGC
ACAGATGGATGCCCTGTGTCGGTCGAGAAGATTGTGAACATCTTCAATGGGACCAGCTGC
CCCAGCCTGGGAGGGAAGCCCAAGCTCTTTTTCATCCAGGCCTGTGGTGGGGAGCAGAAA
GACCATGGGTTTGAGGTGGCCTCCACTTCCCCTGAAGACGAGTCCCCTGGCAGTAACCCC
GAGCCAGATGCCACCCCGTTCCAGGAAGGTTTGAGGACCTTCGACCAGCTGGcCGCCATA
TCTAGTTTGCCCACACCCAGTGACATCTTTGTGTCCTACTCTACTTTCCCAGGTTTTGTTTC
CTGGAGGGACCCCAAGAGTGGCTCCTGGTACGTTGAGACCCTGGACGACATCTTTGAGCA
GTGGGCTCACTCTGAAGACCTGCAGTCCCTCCTGCTTAGGGTCGCTAATGCTGTTTCGGT
GAAAGGGATTTATAAACAGATGCCTGGTTGCTTTAATTTCCTCCGGAAAAAACTTTTCTTTA
AAACATCAGCTAGCAGAGCC
SEQ ID NO: 188: Caspase9 D330E amino acid sequence
VDGFGDVGALESLRGNADLAYILSMEPCGHCLIINNVNFCRESGLRTRTGSNIDCEKLRRRFSS
LHFMVEVKGDLTAKKMVLALLELARQDHGALDCCVVVILSHGCQASHLQFPGAVYGTDGCPVS
VEKIVNIFNGTSCPSLGGKPKLFFIQACGGEQKDHGFEVASTSPEDESPGSNPEPDATPFQEGL
RTFDQLeAISSLPTPSDIFVSYSTFPGFVSWRDPKSGSVVYVETLDDIFEQWAHSEDLQSLLLRV
ANAVSVKGIYKQMPGCFNFLRKKLFFKTSASRA
Sequences for pBPO509
pBP0509-SFG-PSCAscFv.CH2CH3.CD28tm.zeta.MyD88/CD40 sequence
Signal peptide
SEQ ID NO: 189
ATGGAGTTTGGACTTTCTTGGTTGTTTTTGGTGGCAATTCTGAAGGGTGTCCAGTGTAGCA
GG
Signal peptide
SEQ ID NO: 190
MEFGLSWLFLVAILKGVQCSR
bm2B3 variable light chain
SEQ ID NO: 191
GACATCCAGCTGACACAAAGTCCCAGTAGCCTGTCAGCCAGTGTCGGCGATAGGGTGACA
ATTACATGCTCCGCAAGTAGTAGCGTCAGATTCATACACTGGTACCAGCAGAAGCCTGGGA
AGGCCCCAAAGAGGCTTATCTACGATACCAGTAAACTCGCCTCTGGAGTTCCTAGCCGGTT
TTCTGGATCTGGCAGCGGAACTAGCTACACCCTCACAATCTCCAGTCTGCAACCAGAGGA
CTTTGCAACCTACTACTGCCAGCAATGGAGCAGCTCCCCTTTCACCTTTGGGCAGGGTACT
AAGGTGGAGATCAAG
bm2B3 variable light chain
SEQ ID NO: 192
DIQLTQSPSSLSASVGDRVTITCSASSSVRFIHVVYQQKPGKAPKRLIYDTSKLASGVPSRFSGS
GSGTSYTLTISSLQPEDFATYYCQQWSSSPFTFGQGTKVEIK
Flexible linker
SEQ ID NO: 193
GGCGGAGGAAGCGGAGGTGGGGGC
Flexible linker
SEQ ID NO: 194
GGGSGGGG
bm2B3 variable heavy chain
SEQ ID NO: 195
GAGGTGCAGCTTGTAGAGAGCGGGGGAGGCCTCGTACAGCCAGGGGGCTCTCTGCGCCT
GTCATGTGCAGCTTCAGGATTCAATATAAAGGACTATTACATTCACTGGGTACGGCAAGCT
CCCGGTAAGGGCCTGGAATGGATCGGTTGGATCGACCCTGAAAACGGAGATACAGAATTT
GTGCCCAAGTTCCAGGGAAAGGCTACCATGTCTGCCGATACTTCTAAGAATACAGCATACC
TTCAGATGAATTCTCTCCGCGCCGAGGACACAGCCGTGTATTATTGTAAAACGGGAGGGTT
CTGGGGTCAGGGTACCCTTGTGACTGTGTCTTCC
bm2B3 variable heavy chain
SEQ ID NO: 196
EVQLVESGGGLVQPGGSLRLSCAASGFNIKDYYIHVVVRQAPGKGLEWIGWIDPENGDTEFVPK
FQGKATMSADTSKNTAYLQMNSLRAEDTAVYYCKTGGFWGQGTLVTVSS
Linker
SEQ ID NO: 197
GGGGATCCCGCC
Linker
SEQ ID NO: 198
GDPA
IgG1 hinge region
SEQ ID NO: 199
GAGCCCAAATCTCCTGACAAAACTCACACATGCCCA
IgG1 hinge region
SEQ ID NO: 200
EPKSPDKTHTCP
IgG1 CH2 region
SEQ ID NO: 201
CCGTGCCCAGCACCTGAACTCCTGGGGGGACCGTCAGTCTTCCTCTTCCCCCCAAAACCC
AAAGACACCCTCATGATCTCCCGGACCCCTGAGGTCACATGCGTGGTGGTGGACGTGAGC
CACGAAGACCCTGAGGTCAAGTTCAACTGGTATGTGGACGGCGTGGAGGTGCATAATGCA
AAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAA
IgG1 CH2 region
SEQ ID NO: 202
PCPAPELLGGPSVFLFPPKPKDTLMISRTPEVTCVVVDVSHEDPEVKFNVVYVDGVEVHNAKTK
PREEQYNSTYRVVSVLTVLHQDWLNGKEYKCKVSNKALPAPIEKTISKAK
IgG1 CH3 region
SEQ ID NO: 203
GGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGA
GTGGGAGAGCAATGGGCAACCGGAGAACAACTACAAGACCACGCCTCCCGTGCTGGACT
CCGACGGCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCAGAAGA
GCCTCTCCCTGTCTCCGGGTAAA
IgG1 CH3 region
SEQ ID NO: 204
GQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Linker
SEQ ID NO: 205
AAAGATCCCAAA
Linker
SEQ ID NO: 206
KDPK
CD28 transmembrane region
SEQ ID NO: 207
TTTTGGGTGCTGGTGGTGGTTGGTGGAGTCCTGGCTTGCTATAGCTTGCTAGTAACAGTG
GCCTTTATTATT
CD28 transmembrane region
SEQ ID NO: 208
FWVLVVVGGVLACYSLLVTVAFII
Linker
SEQ ID NO: 209
gccggc
Linker
SEQ ID NO: 210
AG
CD3 zeta
SEQ ID NO: 211
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta
SEQ ID NO: 212
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
MyD88
SEQ ID NO: 213
GCCGCTGGGGGCCCAGGCGCCGGATCAGCTGCTCCCGTATCTTCTACTTCTTCTTTGCCG
CTGGCTGCTCTGAACATGCGCGTGAGAAGACGCCTCTCCCTGTTCCTTAACGTTCGCACA
CAAGTCGCTGCCGATTGGACCGCCCTTGCCGAAGAAATGGACTTTGAATACCTGGAAATTA
GACAACTTGAAACACAGGCCGACCCCACTGGCAGACTCCTGGACGCATGGCAGGGAAGA
CCTGGTGCAAGCGTTGGACGGCTCCTGGATCTCCTGACAAAACTGGGACGCGACGACGTA
CTGCTTGAACTCGGACCTAGCATTGAAGAAGACTGCCAAAAATATATCCTGAAACAACAAC
AAGAAGAAGCCGAAAAACCTCTCCAAGTCGCAGCAGTGGACTCATCAGTACCCCGAACAG
CTGAGCTTGCTGGGATTACTACACTCGACGACCCACTCGGACATATGCCTGAAAGATTCGA
CGCTTTCATTTGCTATTGCCCCTCTGACATA
MyD88
SEQ ID NO: 214
AAGGPGAGSAAPVSSTSSLPLAALNMRVRRRLSLFLNVRTQVAADVVTALAEEMDFEYLEIRQL
ETQADPTGRLLDAWQGRPGASVGRLLDLLTKLGRDDVLLELGPSIEEDCQKYILKQQQEEAEK
PLQVAAVDSSVPRTAELAGITTLDDPLGHMPERFDAFICYCPSDI
CD40
SEQ ID NO: 215
AAGAAAGTTGCAAAGAAACCCACAAATAAAGCCCCACACCCTAAACAGGAACCCCAAGAAA
TCAATTTCCCAGATGATCTCCCTGGATCTAATACTGCCGCCCCGGTCCAAGAAACCCTGCA
TGGTTGCCAGCCTGTCACCCAAGAGGACGGAAAAGAATCACGGATTAGCGTACAAGAGAG
ACAATAG
CD40
SEQ ID NO: 216
KKVAKKPTNKAPHPKQEPQEINFPDDLPGSNTAAPVQETLHGCQPVTQEDGKESRISVQERQ*
Sequences for pBP0425
pBP0521-SFG-CD19scFv.CH2CH3.CD28tm.MyD88/CD40.zeta sequence
Signal peptide
SEQ ID NO: 217
ATGGAGTTTGGACTTTCTTGGTTGTTTTTGGTGGCAATTCTGAAGGGTGTCCAGTGTAGCA
GG
Signal peptide
SEQ ID NO: 218
MEFGLSWLFLVAILKGVQCSR
FMC63 variable light chain
SEQ ID NO: 219
GACATCCAGAT
GACACAGACTACATCCTCCCTGTCTGCCTCTCTGGGAGACAGAGTCACCATCAGTTGCAG
GGCAAGTCAGGACATTAGTAAATATTTAAATTGGTATCAGCAGAAACCAGATGGAACTGTTA
AACTCCTGATCTACCATACATCAAGATTACACTCAGGAGTCCCATCAAGGTTCAGTGGCAG
TGGGTCTGGAACAGATTATTCTCTCACCATTAGCAACCTGGAGCAAGAAGATATTGCCACT
TACTTTTGCCAACAGGGTAATACGCTTCCGTACACGTTCGGAGGGGGGACTAAGTTGGAAA
TAACA
FMC63 variable light chain
SEQ ID NO: 220
DIQMTQTTSSLSASLGDRVTISCRASQDISKYLNVVYQQKPDGTVKLLIYHTSRLHSGVPSRFSG
SGSGTDYSLTISNLEQEDIATYFCQQGNTLPYTFGGGTKLEIT
Flexible linker
SEQ ID NO: 221
GGCGGAGGAAGCGGAGGTGGGGGC
Flexible linker
SEQ ID NO: 222
GGGSGGGG
FMC63 variable heavy chain
SEQ ID NO: 223
GAGGTGAAACTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGATTCGCCAGCCT
CCACGAAAGGGTCTGGAGTGGCTGGGAGTAATATGGGGTAGTGAAACCACATACTATAATT
CAGCTCTCAAATCCAGACTGACCATCATCAAGGACAACTCCAAGAGCCAAGTTTTCTTAAAA
ATGAACAGTCTGCAAACTGATGACACAGCCATTTACTACTGTGCCAAACATTATTACTACGG
TGGTAGCTATGCTATGGACTACTGGGGTCAAGGAACCTCAGTCACCGTCTCCTCA
FMC63 variable heavy chain
SEQ ID NO: 224
EVKLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
Linker
SEQ ID NO: 225
GGGGATCCCGCC
Linker
SEQ ID NO: 226
GDPA
IgG1 hinge
SEQ ID NO: 227
GAGCCCAAATCTCCTGACAAAACTCACACATGCCCA
IgG1 hinge
SEQ ID NO: 228
EPKSPDKTHTCP
IgG1 CH2 region
SEQ ID NO: 229
CCGTGCCCAGCACCTGAACTCCTGGGGGGACCGTCAGTCTTCCTCTTCCCCCCAAAACCC
AAAGACACCCTCATGATCTCCCGGACCCCTGAGGTCACATGCGTGGTGGTGGACGTGAGC
CACGAAGACCCTGAGGTCAAGTTCAACTGGTATGTGGACGGCGTGGAGGTGCATAATGCA
AAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAA
IgG1 CH2 region
SEQ ID NO: 230
PCPAPELLGGPSVFLFPPKPKDTLMISRTPEVTCVVVDVSHEDPEVKFNVVYVDGVEVHNAKTK
PREEQYNSTYRVVSVLTVLHQDWLNGKEYKCKVSNKALPAPIEKTISKAK
IgG1 CH3 region
SEQ ID NO: 231
GGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGA
GTGGGAGAGCAATGGGCAACCGGAGAACAACTACAAGACCACGCCTCCCGTGCTGGACT
CCGACGGCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCAGAAGA
GCCTCTCCCTGTCTCCGGGTAAA
IgG1 CH3 region
SEQ ID NO: 232
GQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Linker
SEQ ID NO: 233
AAAGATCCCAAA
Linker
SEQ ID NO: 234
KDPK
CD28 transmembrane region
SEQ ID NO: 235
TTTTGGGTGCTGGTGGTGGTTGGTGGAGTCCTGGCTTGCTATAGCTTGCTAGTAACAGTG
GCCTTTATTATT
CD28 transmembrane region
SEQ ID NO: 236
FWVLVVVGGVLACYSLLVTVAFII
Linker
SEQ ID NO: 237
Ctcgag
Linker
SEQ ID NO: 238
LE
MyD88
SEQ ID NO: 239
ATGGCCGCTGGGGGCCCAGGCGCCGGATCAGCTGCTCCCGTATCTTCTACTTCTTCTTTG
CCGCTGGCTGCTCTGAACATGCGCGTGAGAAGACGCCTCTCCCTGTTCCTTAACGTTCGC
ACACAAGTCGCTGCCGATTGGACCGCCCTTGCCGAAGAAATGGACTTTGAATACCTGGAA
ATTAGACAACTTGAAACACAGGCCGACCCCACTGGCAGACTCCTGGACGCATGGCAGGGA
AGACCTGGTGCAAGCGTTGGACGGCTCCTGGATCTCCTGACAAAACTGGGACGCGACGAC
GTACTGCTTGAACTCGGACCTAGCATTGAAGAAGACTGCCAAAAATATATCCTGAAACAAC
AACAAGAAGAAGCCGAAAAACCTCTCCAAGTCGCAGCAGTGGACTCATCAGTACCCCGAA
CAGCTGAGCTTGCTGGGATTACTACACTCGACGACCCACTCGGACATATGCCTGAAAGATT
CGACGCTTTCATTTGCTATTGCCCCTCTGACATA
MyD88
SEQ ID NO: 240
MAAGGPGAGSAAPVSSTSSLPLAALNMRVRRRLSLFLNVRTQVAADWTALAEEMDFEYLEIRQ
LETQADPTGRLLDAWQGRPGASVGRLLDLLTKLGRDDVLLELGPSIEEDCQKYILKQQQEEAE
KPLQVAAVDSSVPRTAELAGITTLDDPLGHMPERFDAFICYCPSDI
CD40
SEQ ID NO: 241
AAGAAAGTTGCAAAGAAACCCACAAATAAAGCCCCACACCCTAAACAGGAACCCCAAGAAA
TCAATTTCCCAGATGATCTCCCTGGATCTAATACTGCCGCCCCGGTCCAAGAAACCCTGCA
TGGTTGCCAGCCTGTCACCCAAGAGGACGGAAAAGAATCACGGATTAGCGTACAAGAGAG
ACAA
CD40
SEQ ID NO: 242
KKVAKKPTNKAPHPKQEPQEINFPDDLPGSNTAAPVQETLHGCQPVTQEDGKESRISVQERQ
Linker
SEQ ID NO: 243
gcggccgcagTCGAG
Linker
SEQ ID NO: 244
AAAVE
CD3 zeta chain
SEQ ID NO: 245
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGCTAA
CD3 zeta chain
SEQ ID NO: 246
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR*
Sequences for SFG-Myr.MC-2A-CD19.scfv.CD34e.CD8stm.zeta
SFG-Myr.MC.2A.CD19scFv.CD34e.CD8stm.zeta sequence
Myristolation
SEQ ID NO: 247
atggggagtagcaagagcaagcctaaggaccccagccagcgc
Myristolation
SEQ ID NO: 248
MGSSKSKPKDPSQR
Linker
SEQ ID NO: 249
ctcgac
Linker
SEQ ID NO: 250
LD
MyD88
SEQ ID NO: 251
atggctgcaggaggtcccggcgcggggtctgcggccccggtctcctccacatcctcccttcccctggctgctctcaacatgcgagtgcg
gcgccgcctgtctctgttcttgaacgtgcggacacaggtggcggccgactggaccgcgctggcggaggagatggactttgagtacttg
gagatccggcaactggagacacaagcggaccccactggcaggctgctggacgcctggcagggacgccctggcgcctctgtaggc
cgactgctcgatctgcttaccaagctgggccgcgacgacgtgctgctggagctgggacccagcattgaggaggattgccaaaagtat
atcttgaagcagcagcaggaggaggctgagaagcctttacaggtggccgctgtagacagcagtgtcccacggacagcagagctgg
cgggcatcaccacacttgatgaccccctggggcatatgcctgagcgtttcgatgccttcatctgctattgccccagcgacatc
MyD88
SEQ ID NO: 252
MAAGGPGAGSAAPVSSTSSLPLAALNMRVRRRLSLFLNVRTQVAADVVTALAEEMDFEYLEIRQ
LETQADPTGRLLDAWQGRPGASVGRLLDLLTKLGRDDVLLELGPSIEEDCQKYILKQQQEEAE
KPLQVAAVDSSVPRTAELAGITTLDDPLGHMPERFDAFICYCPSDI
Linker
SEQ ID NO: 253
gtcgag
Linker
SEQ ID NO: 254
VE
CD40
SEQ ID NO: 255
aaaaaggtggccaagaagccaaccaataaggccccccaccccaagcaggagccccaggagatcaattttcccgacgatcttcct
ggctccaacactgctgctccagtgcaggagactttacatggatgccaaccggtcacccaggaggatggcaaagagagtcgcatctc
agtgcaggagagacag
CD40
SEQ ID NO: 256
KKVAKKPTNKAPHPKQEPQEINFPDDLPGSNTAAPVQETLHGCQPVTQEDGKESRISVQERQ
Linker
SEQ ID NO: 257
CCGCGG
Linker
SEQ ID NO: 258
PR
T2A sequence
SEQ ID NO: 259
GAAGGCCGAGGGAGCCTGCTGACATGTGGCGATGTGGAGGAAAACCCAGGACCA
T2A sequence
SEQ ID NO: 260
EGRGSLLTCGDVEENPGP
Signal peptide
SEQ ID NO: 261
ATGGAGTTTGGACTTTCTTGGTTGTTTTTGGTGGCAATTCTGAAGGGTGTCCAGTGTAGCA
GG
Signal peptide
SEQ ID NO: 262
MEFGLSWLFLVAILKGVQCSR
FMC63 variable light chain
SEQ ID NO: 263
GACATCCAGATGACACAGACTACATCCTCCCTGTCTGCCTCTCTGGGAGACAGAGTCACCA
TCAGTTGCAGGGCAAGTCAGGACATTAGTAAATATTTAAATTGGTATCAGCAGAAACCAGAT
GGAACTGTTAAACTCCTGATCTACCATACATCAAGATTACACTCAGGAGTCCCATCAAGGTT
CAGTGGCAGTGGGTCTGGAACAGATTATTCTCTCACCATTAGCAACCTGGAGCAAGAAGAT
ATTGCCACTTACTTTTGCCAACAGGGTAATACGCTTCCGTACACGTTCGGAGGGGGGACTA
AGTTGGAAATAACA
FMC63 variable light chain
SEQ ID NO: 264
DIQMTQTTSSLSASLGDRVTISCRASQDISKYLNVVYQQKPDGTVKLLIYHTSRLHSGVPSRFSG
SGSGTDYSLTISNLEQEDIATYFCQQGNTLPYTFGGGTKLEIT
Flexible linker
SEQ ID NO: 265
GGCGGAGGAAGCGGAGGTGGGGGC
Flexible linker
SEQ ID NO: 266
GGGSGGGG
FMC63 variable heavy chain
SEQ ID NO: 267
GAGGTGAAACTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGATTCGCCAGCCT
CCACGAAAGGGTCTGGAGTGGCTGGGAGTAATATGGGGTAGTGAAACCACATACTATAATT
CAGCTCTCAAATCCAGACTGACCATCATCAAGGACAACTCCAAGAGCCAAGTTTTCTTAAAA
ATGAACAGTCTGCAAACTGATGACACAGCCATTTACTACTGTGCCAAACATTATTACTACGG
TGGTAGCTATGCTATGGACTACTGGGGTCAAGGAACCTCAGTCACCGTCTCCTCA
FMC63 variable heavy chain
SEQ ID NO: 268
EVKLQESGPGLVAPSQSLSVTCTVSGVSLPDYGVSWIRQPPRKGLEWLGVIWGSETTYYNSAL
KSRLTIIKDNSKSQVFLKMNSLQTDDTAIYYCAKHYYYGGSYAMDYWGQGTSVTVSS
Linker
SEQ ID NO: 269
GGATCC
Linker
SEQ ID NO: 270
GS
CD34 minimal epitope
SEQ ID NO: 271
GAACTTCCTACTCAGGGGACTTTCTCAAACGTTAGCACAAACGTAAGT
CD34 minimal epitope
SEQ ID NO: 272
ELPTQGTFSNVSTNVS
CD8 alpha stalk domain
SEQ ID NO: 273
CCCGCCCCAAGACCCCCCACACCTGCGCCGACCATTGCTTCTCAACCCCTGAGTTTGAGA
CCCGAGGCCTGCCGGCCAGCTGCCGGCGGGGCCGTGCATACAAGAGGACTCGATTTCGC
TTGCGAC
CD8 alpha stalk domain
SEQ ID NO: 274
PAPRPPTPAPTIASQPLSLRPEACRPAAGGAVHTRGLDFACD
CD8 alpha transmembrane domain
SEQ ID NO: 275
ATCTATATCTGGGCACCTCTCGCTGGCACCTGTGGAGTCCTTCTGCTCAGCCTGGTTATTA
CTCTGTACTGTAATCACCGGAATCGCCGCCGCGTTTGTAAGTGTCCCAGG
CD8 alpha transmembrane domain
SEQ ID NO: 276
IYIWAPLAGTCGVLLLSLVITLYCNHRNRRRVCKCPR
Linker
SEQ ID NO: 277
GTCGAC
Linker
SEQ ID NO: 278
VD
CD3 zeta
SEQ ID NO: 279
AGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGC
CD3 zeta
SEQ ID NO: 280
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
(MyD88 nucleotide sequence)
SEQ ID NO: 281
atggctgcaggaggtcccggcgcggggtctgcggccccggtctcctccacatcctcccttcccctggctgctctcaacatgcgagtgcg
gcgccgcctgtctctgttcttgaacgtgcggacacaggtggcggccgactggaccgcgctggcggaggagatggactttgagtacttg
gagatccggcaactggagacacaagcggaccccactggcaggctgctggacgcctggcagggacgccctggcgcctctgtaggc
cgactgctcgagctgcttaccaagctgggccgcgacgacgtgctgctggagctgggacccagcattgaggaggattgccaaaagta
tatcttgaagcagcagcaggaggaggctgagaagcctttacaggtggccgctgtagacagcagtgtcccacggacagcagagctg
gcgggcatcaccacacttgatgaccccctggggcatatgcctgagcgtttcgatgccttcatctgctattgccccagcgacatccagtttg
tgcaggagatgatccggcaactggaacagacaaactatcgactgaagttgtgtgtgtctgaccgcgatgtcctgcctggcacctgtgtc
tggtctattgctagtgagctcatcgaaaagaggtgccgccggatggtggtggttgtctctgatgattacctgcagagcaaggaatgtgac
ttccagaccaaatttgcactcagcctctctccaggtgcccatcagaagcgactgatccccatcaagtacaaggcaatgaagaaagag
ttccccagcatcctgaggttcatcactgtctgcgactacaccaacccctgcaccaaatcttggttctggactcgccttgccaaggccttgt
ccctgccc
(MyD88 amino acid sequence)
SEQ ID NO: 282
M A A G G P G A G S A A P V S S T S S L P L A A L N M R V R R R L S
L F L N V R T Q V A A D W T A L A E E M D F E Y L E I R Q L E T Q A D
P T G R L L D A W Q G R P G A S V G R L L E L L T K L G R D D V L L E
L G P S I E E D C Q K Y I L K Q Q Q E E A E K P L Q V A A V D S S V P
R T A E L A G I T T L D D P L G H M P E R F D A F I C Y C P S D I Q F
V Q E M I R Q L E Q T N Y R L K L C V S D R D V L P G T C V W S I A S
E L I E K R C R R M V V V V S D D Y L Q S K E C D F Q T K F A L S L S
P G A H Q K R L I P I K Y K A M K K E F P S I L R F I T V C D Y T N P C
T K S W F W T R L A K A L S L P

### Example 17: Development of Improved Therapeutic Cell Dimmer Switch

Therapy using autologous T cells expressing chimeric antigen receptors (CARs) directed toward tumor-associated antigens (TAAs) has had a transformational effect on the treatment of certain types of leukemias (“liquid tumors”) and lymphomas with objective response (OR) rates approaching 90%. Despite their great clinical promise and the predictable accompanying enthusiasm, this success is tempered by the observed high level of on-target, off-tumor adverse events, typical of a cytokine release syndrome (CRS). To maintain the benefit of these revolutionary treatments while minimizing the risk, a chimeric caspase polypeptide-based suicide gene system has been developed, which is based on synthetic ligand-mediated dimerization of a modified Caspase-9 protein, fused to a ligand binding domain, called FKBP12v36. In the presence of the FKBP12v36-binding to the small molecule dimerizer, rimiducid (AP1903), Caspase-9 is activated, leading to rapid apoptosis of target cells. Addition of reduced levels of rimiducid can lead to a tempered rate of killing, allowing the amount of T cell elimination to be regulated from almost nothing to almost full elimination of chimeric caspase-modified T cells. To maximize the utility of this “dimmer” switch, the slope of the dose-response curve should be as gradual as possible; otherwise, administration of the correct dose is challenging. With the current, first generation, clinical iCaspase-9 construct, a dose response curve covering about 1.5 to 2 logs has been observed.

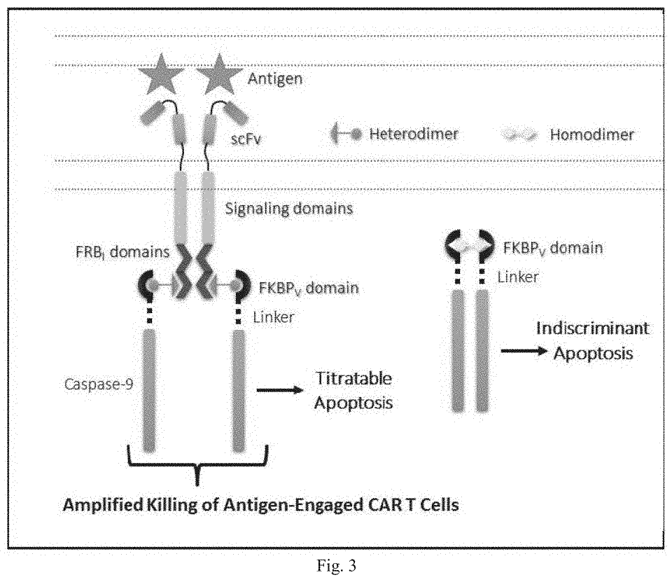

To improve on the therapeutic cell dimmer function, a second level of control may be added to Caspase-9 aggregation, separating rapamycin-driven low levels of aggregation from rimiducid-driven high levels of dimerization. In the first level of control, chimeric caspase polypeptides are recruited by rapamycin/sirolimus (or non-immunosuppressant analog) to a chimeric antigen receptor (CAR), which is modified to contain one or more copies of the 89-amino acid FKBP12-Rapamycin-Binding (FRB) domain (encoded within mTOR) on its carboxy terminus (FIG. 3, left panel). Relative to rimiducid-driven homodimerization of iCaspase-9, it is predicted that the level of Caspase-9 oligomerization would be reduced, both due to the relative affinities of rapamycin-bound FKBP12v36 to FRB (K_{d}˜4 nM) vs rimiducid-bound FKBP12v36 (˜0.1 nM) and due to the “staggered” geometry of the crosslinked proteins. An additional level of “fine-tuning” can be provided at the CAR docking site by changing the number of FRB domains fused to each CAR. Meanwhile, target-dependent specificity will be provided by normal target-driven CAR clustering, which should, in turn, be translated to chimeric caspase polypeptide clustering in the presence of rapamycin. When a maximum level of cell elimination is required, rimiducid can also be administered under the current protocol (i.e., currently 0.4 mg/kg in a 2-hour infusion (FIG. 3, right panel).

### Methods:

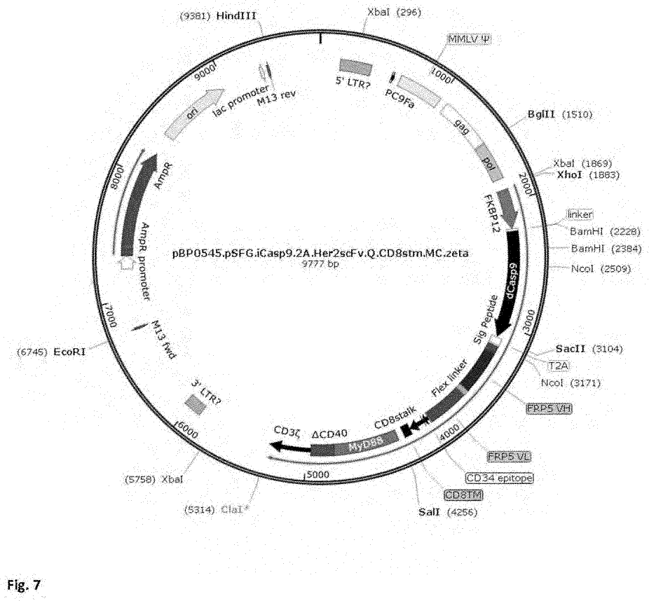

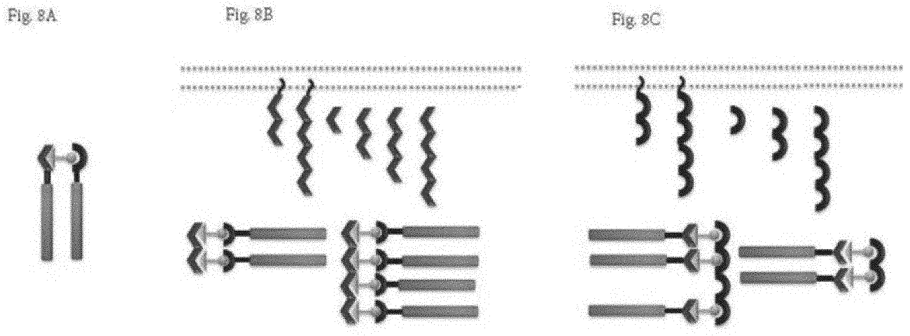

Vectors for rapalog-regulated chimeric caspase polypeptide: The Schreiber lab initially identified the minimal FKBP12-rapamycin binding (FRB) domain from mTOR/FRAP (residues 2025-2114), determining it to have a rapamycin dissociation constant (Kd) about 4 nM (Chen J et al (95) PNAS 92, 4947-51). Subsequent studies identified orthogonal mutants of FRB, such as FRBI (L2098) that bind with relatively high affinity to non-immunosuppressant “bumped” rapamycin analogs (“rapalogs”) (Liberles S D (97) PNAS 94, 7825-30; Bayle J H (06) Chem & Biol 13, 99-107). In order to develop modified MC-CARs that can recruit iC9, the carboxy terminal CD3 zeta domain (from pBP0526) and pBP0545, FIG. 7) are fused to 1 or 2 tandem FRB_{L} domains using a commercially synthesized SalI-MluI fragment that contains MyD88, CD40, and CD3ζ domains to produce vectors pBP0612 and pBP0611, respectively (FIGS. 4 and 5) and Tables 7 and 8. The approach should also be applicable to any CAR construct, including standard, “non-MyD88/CD40” constructs, such as those that include CD28, OX40, and/or 4-1BB, and CD3zeta.

### Results:

As a proof of principal, two tandem FRB, domains were fused to either a 1^{st} generation Her2-CAR or to a 1^{st} generation CD19-CAR co-expressing inducible Caspase-9. 293 cells were transiently transfected with a constitutive reporter plasmid, SRα-SEAP, along with normalized levels of expression plasmids encoding Her2-CAR-FRB2, iCaspase-9, Her2-CAR-FRBₗ2+iCasp9, iC9-CAR(19).FRBₗ2 (coexpressing both CD19-CAR-FRBₗ2 and iCaspase9), or control vector. After 24 hours, cells were washed and distributed into duplicate wells with half-log dilutions of rapamycin or rimiducid. After overnight incubation with drugs, SEAP activity was determined. Interestingly, rapamycin addition led to a broad decrement of SEAP activity up to about a 50% decrease (FIG. 6). This dose-dependent decrease required the presence of both the FRB-tagged CAR and the FKBP-tagged Caspase-9. In contrast, AP1903 decreased SEAP activity to about 20% normal levels at much lower levels of drug, comparable to previous experience. It is likely possible to reduce cell viability with rapamycin and switch to rimiducid for more efficient killing in vivo if necessary. Moreover, on- or off-target-mediated CAR clustering should increase the sensitivity of killing primarily at the site of scFv engagement.

### Additional Permutations of the Hetero-Switch:

Although inducible Caspase-9 has been found to be the fastest and most CID-sensitive suicide gene tested among a large cohort of inducible signaling molecules, many other proteins or protein domains that lead to apoptosis (or related necroptosis, triggering inflammation and necrosis as the means of cell death) could be adapted to homo- or heterodimer-based killing using this approach.

A partial list of proteins that could be activated by rapamycin (or rapalog)-mediated membrane recruitment includes:

Other Caspases (i.e., Caspases 1 to 14, which have been identified in mammals)

Other Caspase-associated adapter molecules, such as FADD (DED), APAF1 (CARD), CRADD/RAIDD (CARD), and ASC (CARD) that function as natural caspase dimerizers (dimerization domains in parentheses).

Pro-apoptotic Bcl-2 Family members, such as Bax and Bak, which can cause mitochondrial depolarization (or mislocalization of anti-apoptotic family members, like Bcl-xL or Bcl-2). RIPK3 or the RIPK1-RHIM domain that can trigger a related form of pro-inflammatory cell death, called necroptosis, due to MLKL-mediated membrane lysis.

Due to its target-dependent level of aggregation, CAR receptors should provide ideal docking sites for rapamycin-mediated recruitment of pro-apoptotic molecules. Nevertheless, many examples exist of multivalent docking site containing FRB domains that could potentially provide rapalog-mediated cell death in the presence of co-expressed chimeric inducible caspase-like molecules.

TABLE 7
iCasp9-2A-ΔCD19-Q-CD28stm-MCz-FRBI2

| | | SEQ | | SEQ |
|---|---|---|---|---|
| | | ID | | ID |
| Fragment | Nucleotide | NO: | Polypeptide | NO: |
| FKBP12v36 | ATGGGAGTGCAGGTGGAGAC | 393 | MGVQVETISPGDGRTFPKRGQTCVV | 394 |
| | TATTAGCCCCGGAGATGGCA | | HYTGMLEDGKKVDSSRDRNKPFKF | |
| | GAACATTCCCCAAAAGAGGAC | | MLGKQEVIRGWEEGVAQMSVGQRA | |
| | AGACTTGCGTCGTGCATTATA | | KLTISPDYAYGATGHPGIIPPHATLVF | |
| | CTGGAATGCTGGAAGACGGC | | DVELLKLE | |
| | AAGAAGGTGGACAGCAGCCG | | | |
| | GGACCGAAACAAGCCCTTCA | | | |
| | AGTTCATGCTGGGGAAGCAG | | | |
| | GAAGTGATCCGGGGCTGGGA | | | |
| | GGAAGGAGTCGCACAGATGT | | | |
| | CAGTGGGACAGAGGGCCAAA | | | |
| | CTGACTATTAGCCCAGACTAC | | | |
| | GCTTATGGAGCAACCGGCCA | | | |
| | CCCCGGGATCATTCCCCCTCA | | | |
| | TGCTACACTGGTCTTCGATGT | | | |
| | GGAGCTGCTGAAGCTGGAA | | | |
| Linker | AGCGGAGGAGGATCCGGA | 395 | SGGGSG | 396 |
| ΔCaspase-9 | GTGGACGGGTTTGGAGATGT | 397 | SEQ ID NO: 300 | 300 |
| | GGGAGCCCTGGAATCCCTGC | | VDGFGDVGALESLRGNADLAYILSM | |
| | GGGGCAATGCCGATCTGGCT | | EPCGHCLIINNVNFCRESGLRTRTGS | |
| | TACATCCTGTCTATGGAGCCT | | NIDCEKLRRRFSSLHFMVEVKGDLTA | |
| | TGCGGCCACTGTCTGATCATT | | KKMVLALLELARQDHGALDCCVVVIL | |
| | AACAATGTGAACTTCTGCAGA | | SHGCQASHLQFPGAVYGTDGCPVS | |
| | GAGAGCGGGCTGCGGACCAG | | VEKIVNIFNGTSCPSLGGKPKLFFIQA | |
| | AACAGGATCCAATATTGACTG | | CGGEQKDHGFEVASTSPEDESPGS | |
| | TGAAAAGCTGCGGAGAAGGT | | NPEPDATPFQEGLRTFDQLDAISSLP | |
| | TCTCTAGTCTGCACTTTATGG | | TPSDIFVSYSTFPGFVSVVRDPKSGS | |
| | TCGAGGTGAAAGGCGATCTG | | WYVETLDDIFEQWAHSEDLQSLLLR | |
| | ACCGCTAAGAAAATGGTGCTG | | VANAVSVKGIYKQMPGCFNFLRKKL | |
| | GCCCTGCTGGAACTGGCTCG | | FFKTSASRA | |
| | GCAGGACCATGGGGCACTGG | | | |
| | ATTGCTGCGTGGTCGTGATCC | | | |
| | TGAGTCACGGCTGCCAGGCT | | | |
| | TCACATCTGCAGTTCCCTGGG | | | |
| | GCAGTCTATGGAACTGACGG | | | |
| | CTGTCCAGTCAGCGTGGAGA | | | |
| | AGATCGTGAACATCTTCAACG | | | |
| | GCACCTCTTGCCCAAGTCTGG | | | |
| | GCGGGAAGCCCAAACTGTTC | | | |
| | TTTATTCAGGCCTGTGGAGGC | | | |
| | GAGCAGAAAGATCACGGCTT | | | |
| | CGAAGTGGCTAGCACCTCCC | | | |
| | CCGAGGACGAATCACCTGGA | | | |
| | AGCAACCCTGAGCCAGATGC | | | |
| | AACCCCCTTCCAGGAAGGCC | | | |
| | TGAGGACATTTGACCAGCTG | | | |
| | GATGCCATCTCAAGCCTGCCC | | | |
| | ACACCTTCTGACATTTTCGTC | | | |
| | TCTTACAGTACTTTCCCTGGA | | | |
| | TTTGTGAGCTGGCGCGATCCA | | | |
| | AAGTCAGGCAGCTGGTACGT | | | |
| | GGAGACACTGGACGATATCTT | | | |
| | TGAGCAGTGGGCCCATTCTG | | | |
| | AAGACCTGCAGAGTCTGCTG | | | |
| | CTGCGAGTGGCCAATGCTGT | | | |
| | CTCTGTGAAGGGGATCTACAA | | | |
| | ACAGATGCCAGGATGCTTCAA | | | |
| | CTTTCTGAGAAAGAAACTGTT | | | |
| | CTTTAAGACCTCCGCATCTAG | | | |
| | GGCC | | | |
| Linker | CCGCGG | 398 | PR | 399 |
| T2A | GAAGGCCGAGGGAGCCTGCT | 400 | EGRGSLLTCGDVEENPGP | 401 |
| | GACATGTGGCGATGTGGAGG | | | |
| | AAAACCCAGGACCA | | | |
| Linker | Ccatgg | 402 | PW | 403 |
| (NcoI) | | | | |
| Sig Peptide | ATGGAGTTTGGACTTTCTTGG | 404 | MEFGLSVVLFLVAILKGVQCSR | 405 |
| | TTGTTTTTGGTGGCAATTCTG | | | |
| | AAGGGTGTCCAGTGTAGCAG | | | |
| | G | | | |
| FMC63-VL | GACATCCAGATGACACAGACT | 406 | DIQMTQTTSSLSASLGDRVTISCRAS | 407 |
| | ACATCCTCCCTGTCTGCCTCT | | QDISKYLNVVYQQKPDGTVKLLIYHTS | |
| | CTGGGAGACAGAGTCACCAT | | RLHSGVPSRFSGSGSGTDYSLTISNL | |
| | CAGTTGCAGGGCAAGTCAGG | | EQEDIATYFCQQGNTLPYTFGGGTK | |
| | ACATTAGTAAATATTTAAATTG | | LEIT | |
| | GTATCAGCAGAAACCAGATG | | | |
| | GAACTGTTAAACTCCTGATCT | | | |
| | ACCATACATCAAGATTACACT | | | |
| | CAGGAGTCCCATCAAGGTTCA | | | |
| | GTGGCAGTGGGTCTGGAACA | | | |
| | GATTATTCTCTCACCATTAGC | | | |
| | AACCTGGAGCAAGAAGATATT | | | |
| | GCCACTTACTTTTGCCAACAG | | | |
| | GGTAATACGCTTCCGTACACG | | | |
| | TTCGGAGGGGGGACTAAGTT | | | |
| | GGAAATAACA | | | |
| Flex-linker | GGCGGAGGAAGCGGAGGTG | 408 | GGGSGGGG | 409 |
| | GGGGC | | | |
| FMC63-VH | GAGGTGAAACTGCAGGAGTC | 410 | EVKLQESGPGLVAPSQSLSVTCTVS | 411 |
| | AGGACCTGGCCTGGTGGCGC | | GVSLPDYGVSWIRQPPRKGLEWLG | |
| | CCTCACAGAGCCTGTCCGTCA | | VIWGSETTYYNSALKSRLTIIKDNSKS | |
| | CATGCACTGTCTCAGGGGTCT | | QVFLKMNSLQTDDTAIYYCAKHYYY | |
| | CATTACCCGACTATGGTGTAA | | GGSYAMDYWGQGTSVTVSS | |
| | GCTGGATTCGCCAGCCTCCA | | | |
| | CGAAAGGGTCTGGAGTGGCT | | | |
| | GGGAGTAATATGGGGTAGTG | | | |
| | AAACCACATACTATAATTCAG | | | |
| | CTCTCAAATCCAGACTGACCA | | | |
| | TCATCAAGGACAACTCCAAGA | | | |
| | GCCAAGTTTTCTTAAAAATGA | | | |
| | ACAGTCTGCAAACTGATGACA | | | |
| | CAGCCATTTACTACTGTGCCA | | | |
| | AACATTATTACTACGGTGGTA | | | |
| | GCTATGCTATGGACTACTGGG | | | |
| | GTCAAGGAACCTCAGTCACC | | | |
| | GTCTCCTCA | | | |
| Linker(Bam | GGATCC | 412 | GS | 413 |
| HI) | | | | |
| CD34 | GAACTTCCTACTCAGGGGACT | 414 | ELPTQGTFSNVSTNVS | 415 |
| epitope | TTCTCAAACGTTAGCACAAAC | | | |
| | GTAAGT | | | |
| CD8a stalk | CCCGCCCCAAGACCCCCCAC | 416 | PAPRPPTPAPTIASQPLSLRPEACRP | 417 |
| | ACCTGCGCCGACCATTGCTTC | | AAGGAVHTRGLDFACD | |
| | TCAACCCCTGAGTTTGAGACC | | | |
| | CGAGGCCTGCCGGCCAGCTG | | | |
| | CCGGCGGGGCCGTGCATACA | | | |
| | AGAGGACTCGATTTCGCTTGC | | | |
| | GAC | | | |
| CD8tm | ATCTATATCTGGGCACCTCTC | 418 | IYIWAPLAGTCGVLLLSLVITLYCNHR | 419 |
| +stop tf | GCTGGCACCTGTGGAGTCCT | | NRRRVCKCPR | |
| | TCTGCTCAGCCTGGTTATTAC | | | |
| | TCTGTACTGTAATCACCGGAA | | | |
| | TCGCCGCCGCGTTTGTAAGT | | | |
| | GTCCCAGG | | | |
| Linker | gtcgac | 420 | VD | 421 |
| (SaII) | | | | |
| MyD88 | ATGGCCGCTGGGGGCCCAGG | 422 | MAAGGPGAGSAAPVSSTSSLPLAAL | 423 |
| | CGCCGGATCAGCTGCTCCCG | | NMRVRRRLSLFLNVRTQVAADVVTAL | |
| | TATCTTCTACTTCTTCTTTGCC | | AEEMDFEYLEIRQLETQADPTGRLLD | |
| | GCTGGCTGCTCTGAACATGC | | AWQGRPGASVGRLLDLLTKLGRDD | |
| | GCGTGAGAAGACGCCTCTCC | | VLLELGPSIEEDCQKYILKQQQEEAE | |
| | CTGTTCCTTAACGTTCGCACA | | KPLQVAAVDSSVPRTAELAGITTLDD | |
| | CAAGTCGCTGCCGATTGGAC | | PLGHMPERFDAFICYCPSDI | |
| | CGCCCTTGCCGAAGAAATGG | | | |
| | ACTTTGAATACCTGGAAATTA | | | |
| | GACAACTTGAAACACAGGCC | | | |
| | GACCCCACTGGCAGACTCCT | | | |
| | GGACGCATGGCAGGGAAGAC | | | |
| | CTGGTGCAAGCGTTGGACGG | | | |
| | CTCCTGGATCTCCTGACAAAA | | | |
| | CTGGGACGCGACGACGTACT | | | |
| | GCTTGAACTCGGACCTAGCAT | | | |
| | TGAAGAAGACTGCCAAAAATA | | | |
| | TATCCTGAAACAACAACAAGA | | | |
| | AGAAGCCGAAAAACCTCTCCA | | | |
| | AGTCGCAGCAGTGGACTCAT | | | |
| | CAGTACCCCGAACAGCTGAG | | | |
| | CTTGCTGGGATTACTACACTC | | | |
| | GACGACCCACTCGGACATAT | | | |
| | GCCTGAAAGATTCGACGCTTT | | | |
| | CATTTGCTATTGCCCCTCTGA | | | |
| | CATA | | | |
| dCD40 | AAGAAAGTTGCAAAGAAACCC | 424 | KKVAKKPTNKAPHPKQEPQEINFPD | 425 |
| | ACAAATAAAGCCCCACACCCT | | DLPGSNTAAPVQETLHGCQPVTQED | |
| | AAACAGGAACCCCAAGAAATC | | GKESRISVQERQ | |
| | AATTTCCCAGATGATCTCCCT | | | |
| | GGATCTAATACTGCCGCCCC | | | |
| | GGTCCAAGAAACCCTGCATG | | | |
| | GTTGCCAGCCTGTCACCCAA | | | |
| | GAGGACGGAAAAGAATCACG | | | |
| | GATTAGCGTACAAGAGAGACA | | | |
| | A | | | |
| CD3z | AGAGTGAAGTTCAGCAGGAG | 426 | RVKFSRSADAPAYQQGQNQLYNEL | 427 |
| | CGCAGACGCCCCCGCGTACC | | NLGRREEYDVLDKRRGRDPEMGGK | |
| | AGCAGGGCCAGAACCAGCTC | | PRRKNPQEGLYNELQKDKMAEAYS | |
| | TATAACGAGCTCAATCTAGGA | | EIGMKGERRRGKGHDGLYQGLSTAT | |
| | CGAAGAGAGGAGTACGATGT | | KDTYDALHMQALPPR | |
| | TTTGGACAAGAGACGTGGCC | | | |
| | GGGACCCTGAGATGGGGGGA | | | |
| | AAGCCGAGAAGGAAGAACCC | | | |
| | TCAGGAAGGCCTGTACAATGA | | | |
| | ACTGCAGAAAGATAAGATGGC | | | |
| | GGAGGCCTACAGTGAGATTG | | | |
| | GGATGAAAGGCGAGCGCCGG | | | |
| | AGGGGCAAGGGGCACGATGG | | | |
| | CCTTTACCAGGGTCTCAGTAC | | | |
| | AGCCACCAAGGACACCTACG | | | |
| | ACGCCCTTCACATGCAAGCTC | | | |
| | TTCCACCTCGt | | | |
| Linker | Acg | 428 | 429 | |
| FRB_{I}\hat{ }\hat{ } | TGGCACGAAGGCCTGGAAGA | 430 | WHEGLEEASRLYFGERNVKGMFEV | 431 |
| | GGCCTCAAGACTTTACTTTGG | | LEPLHAMMERGPQTLKETSFNQAYG | |
| | TGAACGCAACGTTAAAGGCAT | | RDLMEAQEWCRKYMKSGNVKDLLQ | |
| | GTTCGAGGTGCTGGAACCCTT | | AWDLYYHVFRRISK | |
| | GCATGCAATGATGGAGCGAG | | | |
| | GTCCTCAGACACTCAAAGAGA | | | |
| | CATCTTTTAACCAGGCGTATG | | | |
| | GACGGGACCTCATGGAGGCT | | | |
| | CAGGAATGGTGCCGCAAGTA | | | |
| | CATGAAAAGTGGGAATGTGAA | | | |
| | GGATCTGCTGCAAGCATGGG | | | |
| | ATCTGTATTACCACGTGTTTA | | | |
| | GACGGATCAGCAAA | | | |
| Linker(Bsi | Cgtacg | 432 | RT | 433 |
| WI) | | | | |
| FRBI | TGGCATGAAGGGTTGGAAGA | 434 | WHEGLEEASRLYFGERNVKGMFEV | 435 |
| | AGCTTCAAGGCTGTACTTCGG | | LEPLHAMMERGPQTLKETSFNQAYG | |
| | AGAGAGGAACGTGAAGGGCA | | RDLMEAQEWCRKYMKSGNVKDLLQ | |
| | TGTTTGAGGTTCTTGAACCTC | | AWDLYYHVFRRISK* | |
| | TGCACGCCATGATGGAACGG | | | |
| | GGACCGCAGACACTGAAAGA | | | |
| | AACCTCTTTTAATCAGGCCTA | | | |
| | CGGCAGAGACCTGATGGAGG | | | |
| | CCCAAGAATGGTGTAGAAAGT | | | |
| | ATATGAAATCCGGTAACGTGA | | | |
| | AAGACCTGCTCCAGGCCTGG | | | |
| | GACCTTTATTACCATGTGTTC | | | |
| | AGGCGGATCAGTAAGTAA | | | |

TABLE 8

| | | SEQ | | SEQ |
|---|---|---|---|---|
| | | ID | | ID |
| Fragment | Nucleotide | NO: | Polypeptide | NO: |
| FKBP12v36 | ATGGGAGTGCAGGTGGAGA | 436 | MGVQVETISPGDGRTFPKRGQTCVV | 437 |
| | CTATTAGCCCCGGAGATGGC | | HYTGMLEDGKKVDSSRDRNKPFKF | |
| | AGAACATTCCCCAAAAGAGG | | MLGKQEVIRGVVEEGVAQMSVGQRA | |
| | ACAGACTTGCGTCGTGCATT | | KLTISPDYAYGATGHPGIIPPHATLVF | |
| | ATACTGGAATGCTGGAAGAC | | DVELLKLE | |
| | GGCAAGAAGGTGGACAGCA | | | |
| | GCCGGGACCGAAACAAGCC | | | |
| | CTTCAAGTTCATGCTGGGGA | | | |
| | AGCAGGAAGTGATCCGGGG | | | |
| | CTGGGAGGAAGGAGTCGCA | | | |
| | CAGATGTCAGTGGGACAGA | | | |
| | GGGCCAAACTGACTATTAGC | | | |
| | CCAGACTACGCTTATGGAGC | | | |
| | AACCGGCCACCCCGGGATC | | | |
| | ATTCCCCCTCATGCTACACT | | | |
| | GGTCTTCGATGTGGAGCTGC | | | |
| | TGAAGCTGGAA | | | |
| Linker | AGCGGAGGAGGATCCGGA | 438 | SGGGSG | 439 |
| dCaspase9 | GTGGACGGGTTTGGAGATGT | 440 | VDGFGDVGALESLRGNADLAYILSM | 441 |
| | GGGAGCCCTGGAATCCCTG | | EPCGHCLIINNVNFCRESGLRTRTGS | |
| | CGGGGCAATGCCGATCTGG | | NIDCEKLRRRFSSLHFMVEVKGDLTA | |
| | CTTACATCCTGTCTATGGAG | | KKMVLALLELARQDHGALDCCVVVIL | |
| | CCTTGCGGCCACTGTCTGAT | | SHGCQASHLQFPGAVYGTDGCPVS | |
| | CATTAACAATGTGAACTTCTG | | VEKIVNIFNGTSCPSLGGKPKLFFIQA | |
| | CAGAGAGAGCGGGCTGCGG | | CGGEQKDHGFEVASTSPEDESPGS | |
| | ACCAGAACAGGATCCAATAT | | NPEPDATPFQEGLRTFDQLDAISSLP | |
| | TGACTGTGAAAAGCTGCGGA | | TPSDIFVSYSTFPGFVSWRDPKSGS | |
| | GAAGGTTCTCTAGTCTGCAC | | WYVETLDDIFEQWAHSEDLQSLLLR | |
| | TTTATGGTCGAGGTGAAAGG | | VANAVSVKGIYKQMPGCFNFLRKKL | |
| | CGATCTGACCGCTAAGAAAA | | FFKTSASRA | |
| | TGGTGCTGGCCCTGCTGGAA | | | |
| | CTGGCTCGGCAGGACCATG | | | |
| | GGGCACTGGATTGCTGCGT | | | |
| | GGTCGTGATCCTGAGTCACG | | | |
| | GCTGCCAGGCTTCACATCTG | | | |
| | CAGTTCCCTGGGGCAGTCTA | | | |
| | TGGAACTGACGGCTGTCCAG | | | |
| | TCAGCGTGGAGAAGATCGTG | | | |
| | AACATCTTCAACGGCACCTC | | | |
| | TTGCCCAAGTCTGGGCGGG | | | |
| | AAGCCCAAACTGTTCTTTATT | | | |
| | CAGGCCTGTGGAGGCGAGC | | | |
| | AGAAAGATCACGGCTTCGAA | | | |
| | GTGGCTAGCACCTCCCCCGA | | | |
| | GGACGAATCACCTGGAAGCA | | | |
| | ACCCTGAGCCAGATGCAACC | | | |
| | CCCTTCCAGGAAGGCCTGA | | | |
| | GGACATTTGACCAGCTGGAT | | | |
| | GCCATCTCAAGCCTGCCCAC | | | |
| | ACCTTCTGACATTTTCGTCTC | | | |
| | TTACAGTACTTTCCCTGGATT | | | |
| | TGTGAGCTGGCGCGATCCAA | | | |
| | AGTCAGGCAGCTGGTACGT | | | |
| | GGAGACACTGGACGATATCT | | | |
| | TTGAGCAGTGGGCCCATTCT | | | |
| | GAAGACCTGCAGAGTCTGCT | | | |
| | GCTGCGAGTGGCCAATGCT | | | |
| | GTCTCTGTGAAGGGGATCTA | | | |
| | CAAACAGATGCCAGGATGCT | | | |
| | TCAACTTTCTGAGAAAGAAA | | | |
| | CTGTTCTTTAAGACCTCCGC | | | |
| | ATCTAGGGCC | | | |
| Linker | CCGCGG | 442 | PR | 443 |
| (SacII) | | | | |
| T2A | GAGGGCAGGGGAAGTCTTC | 444 | EGRGSLLTCGDVEENPGP | 445 |
| | TAACATGCGGGGACGTGGA | | | |
| | GGAAAATCCCGGGCCC | | | |
| Linker (NcoI) | GCATGCGCCACC | 446 | ACAT | 447 |
| Sig Peptide | ATGGAGTTTGGGTTGTCATG | 448 | MEFGLSVVLFLVAILKGVQCSR | 449 |
| | GTTGTTTCTCGTCGCTATTCT | | | |
| | CAAAGGTGTACAATGCTCCC | | | |
| | GC | | | |
| FRP5-VH | GAAGTCCAATTGCAACAGTC | 450 | EVQLQQSGPELKKPGETVKISCKAS | 451 |
| | AGGCCCCGAATTGAAAAAGC | | GYPFTNYGMNWVKQAPGQGLKWM | |
| | CCGGCGAAACAGTGAAGATA | | GWINTSTGESTFADDFKGRFDFSLE | |
| | TCTTGTAAAGCCTCCGGTTA | | TSANTAYLQINNLKSEDMATYFCAR | |
| | CCCTTTTACGAACTATGGAAT | | WEVYHGYVPYWGQGTTVTVSS | |
| | GAACTGGGTCAAACAAGCCC | | | |
| | CTGGACAGGGATTGAAGTG | | | |
| | GATGGGATGGATCAATACAT | | | |
| | CAACAGGCGAGTCTACCTTC | | | |
| | GCAGATGATTTCAAAGGTCG | | | |
| | CTTTGACTTCTCACTGGAGA | | | |
| | CCAGTGCAAATACCGCCTAC | | | |
| | CTTCAGATTAACAATCTTAAA | | | |
| | AGCGAGGATATGGCAACCTA | | | |
| | CTTTTGCGCAAGATGGGAAG | | | |
| | TTTATCACGGGTACGTGCCA | | | |
| | TACTGGGGACAAGGAACGA | | | |
| | CAGTGACAGTTAGTAGC | | | |
| Flex-linker | GGCGGTGGAGGCTCCGGTG | 452 | GGGGSGGGGSGGGGS | 453 |
| | GAGGCGGCTCTGGAGGAGG | | | |
| | AGGTTCA | | | |
| FRP5VL | GACATCCAATTGACACAATC | 454 | DIQLTQSHKFLSTSVGDRVSITCKAS | 455 |
| | ACACAAATTTCTCTCAACTTC | | QDVYNAVAWYQQKPGQSPKLLIYSA | |
| | TGTAGGAGACAGAGTGAGCA | | SSRYTGVPSRFTGSGSGPDFTFTISS | |
| | TAACCTGCAAAGCATCCCAG | | VQAEDLAVYFCQQHFRTPFTFGSGT | |
| | GACGTGTACAATGCTGTGGC | | KLEIKAL | |
| | TTGGTACCAACAGAAGCCTG | | | |
| | GACAATCCCCAAAATTGCTG | | | |
| | ATTTATTCTGCCTCTAGTAGG | | | |
| | TACACTGGGGTACCTTCTCG | | | |
| | GTTTACGGGCTCTGGGTCCG | | | |
| | GACCAGATTTCACGTTCACA | | | |
| | ATCAGTTCCGTTCAAGCTGA | | | |
| | AGACCTCGCTGTTTATTTTTG | | | |
| | CCAGCAGCACTTCCGAACCC | | | |
| | CTTTTACTTTTGGCTCAGGCA | | | |
| | CTAAGTTGGAAATCAAGGCT | | | |
| | TTG | | | |
| Linker (NsiI) | Atgcat | 456 | MH | 457 |
| CD34 | GAACTTCCTACTCAGGGGAC | 458 | ELPTQGTFSNVSTNVS | 459 |
| epitope | TTTCTCAAACGTTAGCACAAA | | | |
| | CGTAAGT | | | |
| CD8a stalk | CCCGCCCCAAGACCCCCCA | 460 | PAPRPPTPAPTIASQPLSLRPEACRP | 461 |
| | CACCTGCGCCGACCATTGCT | | AAGGAVHTRGLDFACD | |
| | TCTCAACCCCTGAGTTTGAG | | | |
| | ACCCGAGGCCTGCCGGCCA | | | |
| | GCTGCCGGCGGGGCCGTGC | | | |
| | ATACAAGAGGACTCGATTTC | | | |
| | GCTTGCGAC | | | |
| CD8tm | ATCTATATCTGGGCACCTCT | 462 | IYIWAPLAGTCGVLLLSLVITLYCNHR | 463 |
| +stop tf | CGCTGGCACCTGTGGAGTC | | NRRRVCKCPR | |
| | CTTCTGCTCAGCCTGGTTAT | | | |
| | TACTCTGTACTGTAATCACC | | | |
| | GGAATCGCCGCCGCGTTTGT | | | |
| | AAGTGTCCCAGG | | | |
| Linker(SaII) | gtcgac | 464 | VD | 465 |
| MyD88 | ATGGCCGCTGGGGGCCCAG | 466 | MAAGGPGAGSAAPVSSTSSLPLAAL | 467 |
| | GCGCCGGATCAGCTGCTCC | | NMRVRRRLSLFLNVRTQVAADWTAL | |
| | CGTATCTTCTACTTCTTCTTT | | AEEMDFEYLEIRQLETQADPTGRLLD | |
| | GCCGCTGGCTGCTCTGAACA | | AWQGRPGASVGRLLDLLTKLGRDD | |
| | TGCGCGTGAGAAGACGCCT | | VLLELGPSIEEDCQKYILKQQQEEAE | |
| | CTCCCTGTTCCTTAACGTTC | | KPLQVAAVDSSVPRTAELAGITTLDD | |
| | GCACACAAGTCGCTGCCGAT | | PLGHMPERFDAFICYCPSDI | |
| | TGGACCGCCCTTGCCGAAG | | | |
| | AAATGGACTTTGAATACCTG | | | |
| | GAAATTAGACAACTTGAAAC | | | |
| | ACAGGCCGACCCCACTGGC | | | |
| | AGACTCCTGGACGCATGGCA | | | |
| | GGGAAGACCTGGTGCAAGC | | | |
| | GTTGGACGGCTCCTGGATCT | | | |
| | CCTGACAAAACTGGGACGC | | | |
| | GACGACGTACTGCTTGAACT | | | |
| | CGGACCTAGCATTGAAGAAG | | | |
| | ACTGCCAAAAATATATCCTG | | | |
| | AAACAACAACAAGAAGAAGC | | | |
| | CGAAAAACCTCTCCAAGTCG | | | |
| | CAGCAGTGGACTCATCAGTA | | | |
| | CCCCGAACAGCTGAGCTTGC | | | |
| | TGGGATTACTACACTCGACG | | | |
| | ACCCACTCGGACATATGCCT | | | |
| | GAAAGATTCGACGCTTTCAT | | | |
| | TTGCTATTGCCCCTCTGACA | | | |
| | TA | | | |
| dCD40 | AAGAAAGTTGCAAAGAAACC | 468 | KKVAKKPTNKAPHPKQEPQEINFPD | 469 |
| | CACAAATAAAGCCCCACACC | | DLPGSNTAAPVQETLHGCQPVTQED | |
| | CTAAACAGGAACCCCAAGAA | | GKESRISVQERQ | |
| | ATCAATTTCCCAGATGATCTC | | | |
| | CCTGGATCTAATACTGCCGC | | | |
| | CCCGGTCCAAGAAACCCTGC | | | |
| | ATGGTTGCCAGCCTGTCACC | | | |
| | CAAGAGGACGGAAAAGAATC | | | |
| | ACGGATTAGCGTACAAGAGA | | | |
| | GACAA | | | |
| CD3z | AGAGTGAAGTTCAGCAGGAG | 470 | RVKFSRSADAPAYQQGQNQLYNEL | 471 |
| | CGCAGACGCCCCCGCGTAC | | NLGRREEYDVLDKRRGRDPEMGGK | |
| | CAGCAGGGCCAGAACCAGC | | PRRKNPQEGLYNELQKDKMAEAYS | |
| | TCTATAACGAGCTCAATCTA | | EIGMKGERRRGKGHDGLYQGLSTAT | |
| | GGACGAAGAGAGGAGTACG | | KDTYDALHMQALPPR | |
| | ATGTTTTGGACAAGAGACGT | | | |
| | GGCCGGGACCCTGAGATGG | | | |
| | GGGGAAAGCCGAGAAGGAA | | | |
| | GAACCCTCAGGAAGGCCTGT | | | |
| | ACAATGAACTGCAGAAAGAT | | | |
| | AAGATGGCGGAGGCCTACA | | | |
| | GTGAGATTGGGATGAAAGGC | | | |
| | GAGCGCCGGAGGGGCAAGG | | | |
| | GGCACGATGGCCTTTACCAG | | | |
| | GGTCTCAGTACAGCCACCAA | | | |
| | GGACACCTACGACGCCCTTC | | | |
| | ACATGCAAGCTCTTCCACCT | | | |
| | CGt | | | |
| Linker | Acg | 472 | T | 473 |
| FRBI\hat{ }\hat{ } | TGGCACGAAGGCCTGGAAG | 474 | WHEGLEEASRLYFGERNVKGMFEV | 475 |
| | AGGCCTCAAGACTTTACTTT | | LEPLHAMMERGPQTLKETSFNQAYG | |
| | GGTGAACGCAACGTTAAAGG | | RDLMEAQEWCRKYMKSGNVKDLLQ | |
| | CATGTTCGAGGTGCTGGAAC | | AWDLYYHVFRRISK | |
| | CCTTGCATGCAATGATGGAG | | | |
| | CGAGGTCCTCAGACACTCAA | | | |
| | AGAGACATCTTTTAACCAGG | | | |
| | CGTATGGACGGGACCTCATG | | | |
| | GAGGCTCAGGAATGGTGCC | | | |
| | GCAAGTACATGAAAAGTGGG | | | |
| | AATGTGAAGGATCTGCTGCA | | | |
| | AGCATGGGATCTGTATTACC | | | |
| | ACGTGTTTAGACGGATCAGC | | | |
| | AAA | | | |
| Linker | Cgtacg | 476 | RT | 477 |
| (BsiWI) | | | | |
| FRBI | TGGCATGAAGGGTTGGAAGA | 478 | WHEGLEEASRLYFGERNVKGMFEV | 479 |
| | AGCTTCAAGGCTGTACTTCG | | LEPLHAMMERGPQTLKETSFNQAYG | |
| | GAGAGAGGAACGTGAAGGG | | RDLMEAQEWCRKYMKSGNVKDLLQ | |
| | CATGTTTGAGGTTCTTGAAC | | AWDLYYHVFRRISK* | |
| | CTCTGCACGCCATGATGGAA | | | |
| | CGGGGACCGCAGACACTGA | | | |
| | AAGAAACCTCTTTTAATCAG | | | |
| | GCCTACGGCAGAGACCTGAT | | | |
| | GGAGGCCCAAGAATGGTGT | | | |
| | AGAAAGTATATGAAATCCGG | | | |
| | TAACGTGAAAGACCTGCTCC | | | |
| | AGGCCTGGGACCTTTATTAC | | | |
| | CATGTGTTCAGGCGGATCAG | | | |
| | TAAGTAA | | | |

TABLE 9
pBP0545.pSFG.iCasp9.2A.Her2scFv.Q.CD8stm.MC-zeta

| | | SEQ. | | SEQ. |
|---|---|---|---|---|
| | | ID | | ID |
| Fragment | Nucleotide | NO: | Polypeptide | NO: |
| Kozak | GCCACC | 480 | N/A | 481 |
| (ribosome- | | | | |
| binding | | | | |
| seq.) | | | | |
| FKBP12v36 | ATGGGAGTGCAGGTGGAGA | 481 | MGVQVETISPGDGRTFPKRGQTCVV | 482 |
| | CTATTAGCCCCGGAGATGGC | | HYTGMLEDGKKVDSSRDRNKPFKF | |
| | AGAACATTCCCCAAAAGAGG | | MLGKQEVIRGVVEEGVAQMSVGQRA | |
| | ACAGACTTGCGTCGTGCATT | | KLTISPDYAYGATGHPGIIPPHATLVF | |
| | ATACTGGAATGCTGGAAGAC | | DVELLKLE | |
| | GGCAAGAAGGTGGACAGCA | | | |
| | GCCGGGACCGAAACAAGCC | | | |
| | CTTCAAGTTCATGCTGGGGA | | | |
| | AGCAGGAAGTGATCCGGGG | | | |
| | CTGGGAGGAAGGAGTCGCA | | | |
| | CAGATGTCAGTGGGACAGAG | | | |
| | GGCCAAACTGACTATTAGCC | | | |
| | CAGACTACGCTTATGGAGCA | | | |
| | ACCGGCCACCCCGGGATCAT | | | |
| | TCCCCCTCATGCTACACTGG | | | |
| | TCTTCGATGTGGAGCTGCTG | | | |
| | AAGCTGGAA | | | |
| Linker | AGCGGAGGAGGATCCGGA | 483 | SGGGSG | 484 |
| ΔCaspase9 | GTGGACGGGTTTGGAGATGT | 485 | VDGFGDVGALESLRGNADLAYILSM | 486 |
| | GGGAGCCCTGGAATCCCTG | | EPCGHCLIINNVNFCRESGLRTRTGS | |
| | CGGGGCAATGCCGATCTGG | | NIDCEKLRRRFSSLHFMVEVKGDLTA | |
| | CTTACATCCTGTCTATGGAG | | KKMVLALLELARQDHGALDCCVVVIL | |
| | CCTTGCGGCCACTGTCTGAT | | SHGCQASHLQFPGAVYGTDGCPVS | |
| | CATTAACAATGTGAACTTCTG | | VEKIVNIFNGTSCPSLGGKPKLFFIQA | |
| | CAGAGAGAGCGGGCTGCGG | | CGGEQKDHGFEVASTSPEDESPGS | |
| | ACCAGAACAGGATCCAATAT | | NPEPDATPFQEGLRTFDQLDAISSLP | |
| | TGACTGTGAAAAGCTGCGGA | | TPSDIFVSYSTFPGFVSWRDPKSGS | |
| | GAAGGTTCTCTAGTCTGCAC | | WYVETLDDIFEQWAHSEDLQSLLLR | |
| | TTTATGGTCGAGGTGAAAGG | | VANAVSVKGIYKQMPGCFNFLRKKL | |
| | CGATCTGACCGCTAAGAAAA | | FFKTSASRA | |
| | TGGTGCTGGCCCTGCTGGAA | | | |
| | CTGGCTCGGCAGGACCATG | | | |
| | GGGCACTGGATTGCTGCGTG | | | |
| | GTCGTGATCCTGAGTCACGG | | | |
| | CTGCCAGGCTTCACATCTGC | | | |
| | AGTTCCCTGGGGCAGTCTAT | | | |
| | GGAACTGACGGCTGTCCAGT | | | |
| | CAGCGTGGAGAAGATCGTGA | | | |
| | ACATCTTCAACGGCACCTCT | | | |
| | TGCCCAAGTCTGGGCGGGA | | | |
| | AGCCCAAACTGTTCTTTATTC | | | |
| | AGGCCTGTGGAGGCGAGCA | | | |
| | GAAAGATCACGGCTTCGAAG | | | |
| | TGGCTAGCACCTCCCCCGAG | | | |
| | GACGAATCACCTGGAAGCAA | | | |
| | CCCTGAGCCAGATGCAACCC | | | |
| | CCTTCCAGGAAGGCCTGAGG | | | |
| | ACATTTGACCAGCTGGATGC | | | |
| | CATCTCAAGCCTGCCCACAC | | | |
| | CTTCTGACATTTTCGTCTCTT | | | |
| | ACAGTACTTTCCCTGGATTTG | | | |
| | TGAGCTGGCGCGATCCAAAG | | | |
| | TCAGGCAGCTGGTACGTGGA | | | |
| | GACACTGGACGATATCTTTG | | | |
| | AGCAGTGGGCCCATTCTGAA | | | |
| | GACCTGCAGAGTCTGCTGCT | | | |
| | GCGAGTGGCCAATGCTGTCT | | | |
| | CTGTGAAGGGGATCTACAAA | | | |
| | CAGATGCCAGGATGCTTCAA | | | |
| | CTTTCTGAGAAAGAAACTGTT | | | |
| | CTTTAAGACCTCCGCATCTA | | | |
| | GGGCC | | | |
| Linker | CCGCGG | 487 | PR | 488 |
| (SacII) | | | | |
| T2A | GAGGGCAGGGGAAGTCTTCT | 489 | EGRGSLLTCGDVEENPGP | 490 |
| | AACATGCGGGGACGTGGAG | | | |
| | GAAAATCCCGGGCCC | | | |
| Linker | GCATGCGCCACC | 491 | ACAT | 492 |
| (NcoI) | | | | |
| Sig Peptide | ATGGAGTTTGGGTTGTCATG | 493 | MEFGLSWLFLVAILKGVQCSR | 494 |
| | GTTGTTTCTCGTCGCTATTCT | | | |
| | CAAAGGTGTACAATGCTCCC | | | |
| | GC | | | |
| FRP5- | GAAGTCCAATTGCAACAGTC | 495 | EVQLQQSGPELKKPGETVKISCKAS | 496 |
| VH(anti- | AGGCCCCGAATTGAAAAAGC | | GYPFTNYGMNWVKQAPGQGLKWM | |
| Her2) | CCGGCGAAACAGTGAAGATA | | GWINTSTGESTFADDFKGRFDFSLE | |
| | TCTTGTAAAGCCTCCGGTTA | | TSANTAYLQINNLKSEDMATYFCAR | |
| | CCCTTTTACGAACTATGGAAT | | WEVYHGYVPYWGQGTTVTVSS | |
| | GAACTGGGTCAAACAAGCCC | | | |
| | CTGGACAGGGATTGAAGTGG | | | |
| | ATGGGATGGATCAATACATC | | | |
| | AACAGGCGAGTCTACCTTCG | | | |
| | CAGATGATTTCAAAGGTCGC | | | |
| | TTTGACTTCTCACTGGAGAC | | | |
| | CAGTGCAAATACCGCCTACC | | | |
| | TTCAGATTAACAATCTTAAAA | | | |
| | GCGAGGATATGGCAACCTAC | | | |
| | TTTTGCGCAAGATGGGAAGT | | | |
| | TTATCACGGGTACGTGCCAT | | | |
| | ACTGGGGACAAGGAACGACA | | | |
| | GTGACAGTTAGTAGC | | | |
| Flex-linker | GGCGGTGGAGGCTCCGGTG | 497 | GGGGSGGGGSGGGGS | 498 |
| | GAGGCGGCTCTGGAGGAGG | | | |
| | AGGTTCA | | | |
| FRP5VL(anti- | GACATCCAATTGACACAATC | 499 | DIQLTQSHKFLSTSVGDRVSITCKAS | 500 |
| Her2) | ACACAAATTTCTCTCAACTTC | | QDVYNAVAWYQQKPGQSPKLLIYSA | |
| | TGTAGGAGACAGAGTGAGCA | | SSRYTGVPSRFTGSGSGPDFTFTISS | |
| | TAACCTGCAAAGCATCCCAG | | VQAEDLAVYFCQQHFRTPFTFGSGT | |
| | GACGTGTACAATGCTGTGGC | | KLEIKAL | |
| | TTGGTACCAACAGAAGCCTG | | | |
| | GACAATCCCCAAAATTGCTG | | | |
| | ATTTATTCTGCCTCTAGTAGG | | | |
| | TACACTGGGGTACCTTCTCG | | | |
| | GTTTACGGGCTCTGGGTCCG | | | |
| | GACCAGATTTCACGTTCACA | | | |
| | ATCAGTTCCGTTCAAGCTGA | | | |
| | AGACCTCGCTGTTTATTTTTG | | | |
| | CCAGCAGCACTTCCGAACCC | | | |
| | CTTTTACTTTTGGCTCAGGCA | | | |
| | CTAAGTTGGAAATCAAGGCT | | | |
| | TTG | | | |
| Linker(Nsil) | Atgcat | 501 | MH | 502 |
| CD34 | GAACTTCCTACTCAGGGGAC | 503 | ELPTQGTFSNVSTNVS | 504 |
| epitope | TTTCTCAAACGTTAGCACAAA | | | |
| | CGTAAGT | | | |
| CD8a stalk | CCCGCCCCAAGACCCCCCA | 505 | PAPRPPTPAPTIASQPLSLRPEACRP | 506 |
| | CACCTGCGCCGACCATTGCT | | AAGGAVHTRGLDFACD | |
| | TCTCAACCCCTGAGTTTGAG | | | |
| | ACCCGAGGCCTGCCGGCCA | | | |
| | GCTGCCGGCGGGGCCGTGC | | | |
| | ATACAAGAGGACTCGATTTC | | | |
| | GCTTGCGAC | | | |
| CD8tm | ATCTATATCTGGGCACCTCT | 507 | IYIWAPLAGTCGVLLLSLVITLYCNHR | 508 |
| +stop tf | CGCTGGCACCTGTGGAGTCC | | NRRRVCKCPR | |
| | TTCTGCTCAGCCTGGTTATTA | | | |
| | CTCTGTACTGTAATCACCGG | | | |
| | AATCGCCGCCGCGTTTGTAA | | | |
| | GTGTCCCAGG | | | |
| Linker | gtcgac | 509 | VD | 510 |
| (SaII) | | | | |
| MyD88 | ATGGCCGCTGGGGGCCCAG | 511 | MAAGGPGAGSAAPVSSTSSLPLAAL | 512 |
| | GCGCCGGATCAGCTGCTCC | | NMRVRRRLSLFLNVRTQVAADWTAL | |
| | CGTATCTTCTACTTCTTCTTT | | AEEMDFEYLEIRQLETQADPTGRLLD | |
| | GCCGCTGGCTGCTCTGAACA | | AWQGRPGASVGRLLDLLTKLGRDD | |
| | TGCGCGTGAGAAGACGCCTC | | VLLELGPSIEEDCQKYILKQQQEEAE | |
| | TCCCTGTTCCTTAACGTTCG | | KPLQVAAVDSSVPRTAELAGITTLDD | |
| | CACACAAGTCGCTGCCGATT | | PLGHMPERFDAFICYCPSDI | |
| | GGACCGCCCTTGCCGAAGAA | | | |
| | ATGGACTTTGAATACCTGGA | | | |
| | AATTAGACAACTTGAAACACA | | | |
| | GGCCGACCCCACTGGCAGA | | | |
| | CTCCTGGACGCATGGCAGG | | | |
| | GAAGACCTGGTGCAAGCGTT | | | |
| | GGACGGCTCCTGGATCTCCT | | | |
| | GACAAAACTGGGACGCGAC | | | |
| | GACGTACTGCTTGAACTCGG | | | |
| | ACCTAGCATTGAAGAAGACT | | | |
| | GCCAAAAATATATCCTGAAAC | | | |
| | AACAACAAGAAGAAGCCGAA | | | |
| | AAACCTCTCCAAGTCGCAGC | | | |
| | AGTGGACTCATCAGTACCCC | | | |
| | GAACAGCTGAGCTTGCTGGG | | | |
| | ATTACTACACTCGACGACCC | | | |
| | ACTCGGACATATGCCTGAAA | | | |
| | GATTCGACGCTTTCATTTGCT | | | |
| | ATTGCCCCTCTGACATA | | | |
| dCD40 | AAGAAAGTTGCAAAGAAACC | 513 | KKVAKKPTNKAPHPKQEPQEINFPD | 514 |
| | CACAAATAAAGCCCCACACC | | DLPGSNTAAPVQETLHGCQPVTQED | |
| | CTAAACAGGAACCCCAAGAA | | GKESRISVQERQ | |
| | ATCAATTTCCCAGATGATCTC | | | |
| | CCTGGATCTAATACTGCCGC | | | |
| | CCCGGTCCAAGAAACCCTGC | | | |
| | ATGGTTGCCAGCCTGTCACC | | | |
| | CAAGAGGACGGAAAAGAATC | | | |
| | ACGGATTAGCGTACAAGAGA | | | |
| | GACAA | | | |
| CD3z | AGAGTGAAGTTCAGCAGGAG | 515 | RVKFSRSADAPAYQQGQNQLYNEL | 516 |
| | CGCAGACGCCCCCGCGTAC | | NLGRREEYDVLDKRRGRDPEMGGK | |
| | CAGCAGGGCCAGAACCAGC | | PRRKNPQEGLYNELQKDKMAEAYS | |
| | TCTATAACGAGCTCAATCTAG | | EIGMKGERRRGKGHDGLYQGLSTAT | |
| | GACGAAGAGAGGAGTACGAT | | KDTYDALHMQALPPR* | |
| | GTTTTGGACAAGAGACGTGG | | | |
| | CCGGGACCCTGAGATGGGG | | | |
| | GGAAAGCCGAGAAGGAAGA | | | |
| | ACCCTCAGGAAGGCCTGTAC | | | |
| | AATGAACTGCAGAAAGATAA | | | |
| | GATGGCGGAGGCCTACAGT | | | |
| | GAGATTGGGATGAAAGGCGA | | | |
| | GCGCCGGAGGGGCAAGGGG | | | |
| | CACGATGGCCTTTACCAGGG | | | |
| | TCTCAGTACAGCCACCAAGG | | | |
| | ACACCTACGACGCCCTTCAC | | | |
| | ATGCAAGCTCTTCCACCTCG | | | |
| | Ttga | | | |

Methods discussed herein, including, but not limited to, methods for constructing vectors, assays for activity or function, administration to patients, transfecting or transforming cells, assay, and methods for monitoring patients may also be found in the following patents and patent applications, which are hereby incorporated by reference herein in their entirety.

U.S. patent application Ser. No. 14/210,034, titled METHODS FOR CONTROLLING T CELL PROLIFERATION, filed Mar. 13, 2014; U.S. patent application Ser. No. 13/112,739, filed May 20, 2011, issued as U.S. Pat. No. 9,089,520, Jul. 28, 2015, and entitled METHODS FOR INDUCING SELECTIVE APOPTOSIS; U.S. patent application Ser. No. 14/622,018, filed Feb. 13, 2014, titled METHODS FOR ACTIVATING T CELLS USING AN INDUCIBLE CHIMERIC POLYPEPTIDE; U.S. patent application Ser. No. 13/112,739, filed May 20, 2011, titled METHODS FOR INDUCING SELECTIVE APOPTOSIS; U.S. patent application Ser. No. 13/792,135, filed Mar. 10, 2013, titled MODIFIED CASPASE POLYPEPTIDES AND USES THEREOF; U.S. patent application Ser. No. 14/296,404, filed Jun. 4, 2014, titled METHODS FOR INDUCING PARTIAL APOPTOSIS USING CASPASE POLYPEPTIDES; U.S. Provisional Patent Application Ser. No. 62/044,885, filed Sep. 2, 2014, and U.S. patent application Ser. No. 14/842,710, filed Sep. 1, 2015, each titled COSTIMULATION OF CHIMERIC ANTIGEN RECEPTORS BY MyD88 AND CD40 POLYPEPTIDES; U.S. patent application Ser. No. 14/640,554, filed 6 Mar. 2015, titled CASPASE POLYPEPTIDES HAVING MODIFIED ACTIVITY AND USES THEREOF; U.S. Pat. No. 7,404,950, issued Jun. 29, 2008, to Spencer, D. et al., U.S. patent application Ser. No. 12/445,939 by Spencer, D., et al., filed Oct. 26, 2010; U.S. patent application Ser. No. 12/563,991 by Spencer, D., et al., filed Sep. 21, 2009; 13/087,329 by Slawin, K., et al., filed Apr. 14, 2011; 13/763,591 by Spencer, D., et al., filed Feb. 8, 2013; and International Patent Application Number PCT/US2014/022004, filed 7 Mar. 2014, published as PCT/US2014/022004 on 9 Oct. 2014, titled MODIFIED CASPASE POLYPEPTIDES AND USES THEREOF.

### Example 18: FRB-Based Scaffold Assembly and Activation of iCaspase-9

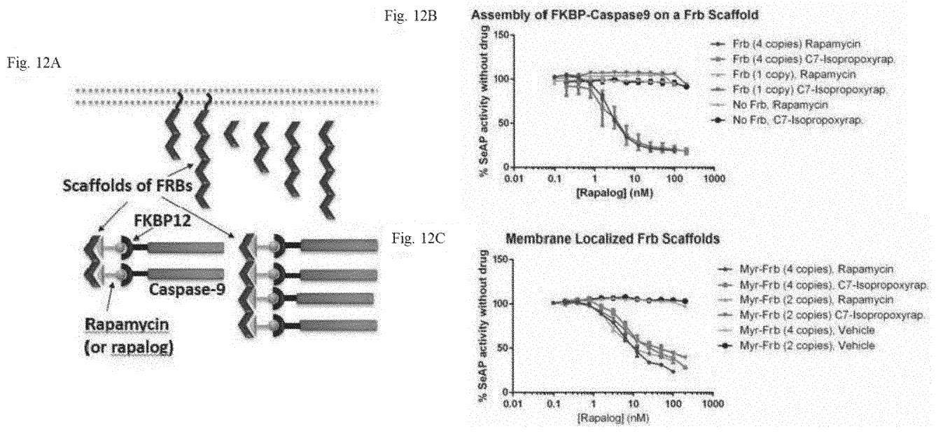

To determine if iCaspase-9 could be aggregated by tandem multimers of FRB_{L}, one to four tandem copies of FRB_{L} were subcloned into an expression vector, pSH1, driving transgene expression from an SRα promoter. A subset of constructs also contained the myristoylation-targeting domain from v-Src for membrane localization of the FRB-scaffold (FIG. 12A). 293 cells were transfected with the SRα-SEAP reporter plasmid along with FKBP12-ΔCaspase-9 (iCaspase-9/iC9), plus 1 of several FRB-based, non-myristoylated scaffold proteins containing 0, 1, or 4 tandem copies of FRB_{L}. Addition of either rapamycin or analog, C7-isopropoxy-rapamycin, created by the method of Luengo et al., (Luengo J I (95) Chem & Biol 2, 471-81. Luengo J I (94) J. Org Chem 59: 6512-13), led to a diminution of reporter activity when the 4×FRB construct was present, consistent with cell death, as predicted (FIG. 8B, 10D, 10E) with a IC₅₀˜3 nM (FIG. 12B). Addition of rapamycin had no effect on reporter activity when only 1 (or 0) FRB domain was present, which would preclude oligomerization of iCasp9 (FIG. 10C). Similar results were obtained when the FRB-scaffold was myristoylated (FIG. 12C) to localize the scaffold to the plasma membrane. Thus, the Caspase-9 polypeptide can be activated with rapamycin or analogs when oligomerized on a FRB-based scaffold.

### Example 19: FKBP12-Based Scaffolds Assemble and Activate FRB-ΔCaspase-9

To determine if the polarity of heterodimerization and Caspase-9 assembly could be reversed, one to four 1 to 4 tandem copies of FKBP12 were subcloned into expression vector, pSH1, as above. (FIG. 13A). As above, 293 cells were transfected with the SRα-SEAP reporter plasmid along with FRBL-ΔCaspase-9, plus a non-myristoylated scaffold protein containing 1 or 4 tandem copies of FKBP12. Addition of either rapamycin or analog, C7-isopropoxy-rapamycin, led to a diminution of reporter activity when the 4×FRB_{L} construct was present, consistent with cell death with a IC₅₀˜3 nM (FIG. 13B). Addition of rapamycin had no effect on reporter activity when only 1 (or 0) FKBP domain was present, similar to the results in FIG. 12. Thus, Caspase-9 can be activated with rapamycin or analogs when oligomerized on a FRB or FKBP12-based scaffold.

### Example 20: FRB-Based Scaffold Assembly and Activation of iCaspase-9 in Primary T Cells

To determine if iCaspase-9 could be aggregated by tandem multimers of FRB_{L} in primary, non-transformed T cells, zero to three 3 tandem copies of FRBL were subcloned into a retroviral expression vector, pBP0220-pSFG-iC9.T2A-ΔCD19, encoding Caspase-9 (iC9) along with a non-signaling truncated version of CD19 that served as a surface marker. The resulting unified plasmid vectors, named pBP0756-iC9.T2A-ΔCD19.P2A-FRB_{L}, pBP0755-iC9.T2A-ΔCD19.P2A-FRB_{L}2, and pBP0757-iC9.T2A-ΔCD19.P2A-FRB_{L}3, were subsequently used to make infectious γ-retroviruses (γ-RVs) encoding scaffolds of 1, 2 or 3 tandem FRB_{L} domains, respectively.

T cells from 3 different donors were transduced with the vectors and plated with varying rapamycin dilutions. After 24 and 48 hours, cell aliquots were harvested, stained with anti-CD19 APC and analyzed by flow cytometry. Cells were initially gated on live lymphocytes by FSC vs SSC and then plotted as a CD19 histogram and subgated for high, medium and low expression within the CD19⁺ gate. Line graphs were prepared to represent the relative percentage of the total cell population that express high levels of CD19, normalized to the no “0” drug control (FIG. 14). Similar to the surrogate SEAP reporter assay performed in transformed epithelial cells, as rapamycin concentration increased, the percentage of CD19hi cells decreased in cells expressing Caspase-9 and FRB_{L}2 or FRB_{L}3, but not in cells expressing Caspase-9 along with 0 or 1 FRB_{L} domains, indicating that rapamycin induces heterodimerization between the FRB-based scaffolds and iCaspase9, leading to Caspase-9 dimerization and cell death. Similar results were seen when rapamycin was replaced with C7-isopropoxyrapamycin.

### Example 21: FRB-Based Scaffolds Attached to Signaling Molecules can Dimerize and Activate iCaspase-9

To determine if multimers of FRB would still act as a recruitment scaffold to enable rapalog-mediated Caspase-9 dimerization when attached to another signaling domain, 1 or 2 FRB_{L} domains were fused to the potent chimeric stimulatory molecule, MyD88/CD40, to derive iMC.FRB_{L} (pBP0655) and iMC.FRB_{L}2 (pBP0498), respectively (FIG. 9B). As an initial test, 293 cells were transiently transfected with reporter plasmid SRα-SEAP, Caspase-9, a 1st generation anti-HER2 CAR (pBP0488) and (pBP0655 or pBP0498) (FIG. 15). Control transfections contained Caspase-9 (pBP0044) alone or eGFP expression vector (pBP0047). In the presence of rimiducid, Caspase-9-containing cells, but not control eGFP-cells, were killed by Caspase-9 homodimerization as usual, reflected by diminution of SEAP activity (FIG. 15, left); however, rapamycin only triggered SEAP reduction in cells expressing iMC.FRB_{L}2 and Caspase-9, but not cells expressing iMC.FRB_{L} and Caspase-9, or control cells. Thus, heterodimerizer-mediated activation of Caspase-9 is possible in cells containing multimers of FRB_{L} fused to distinct proteins, such as MyD88/CD40.

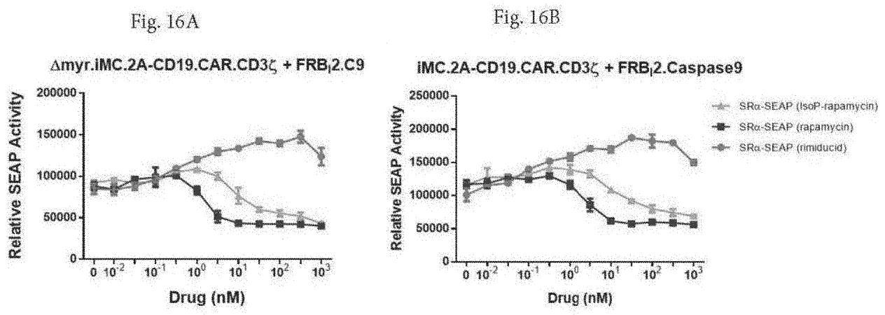

In a second test for rapalog-mediated scaffold-based activation of Caspase-9, 293 cells were transiently transfected with SRα-SEAP reporter plasmid, plus myristoylated or non-myristoylated inducible iMC co-expressed with 1st generation anti-CD19 CAR, plus FRB_{L}2-fused Caspase-9 (plasmid pBP0467) (FIG. 16). After 24 hours, cells were treated with log dilutions of rimiducid, rapamycin, or C7-isopropoxy (IsoP)-rapamycin. Unlike FKBP12-linked Caspase-9 (iC9), FRB_{L}2-Caspase-9 is not activated by rimiducid; however, it is activated by rapamycin or C7-isopropoxy-rapamycin when tandem FKBPs are present. Thus, rapamycin and analogs can activate Caspase-9 via a molecular scaffold comprised of FRB or FKBP12 domains.

### Example 22: The iMC “Switch”, FKBPx2.MyD88.CD40, Creates a Scaffold for FRB_{L}2.Caspase9 in the Presence of Rapamycin to Induce Cell Death

The use of iMC as an FKBP12-based scaffold for activating FRB_{L}2-Caspase-9 was tested in primary T cells (FIG. 17). Primary T cells (2 donors) were transduced with γ-RVs derived from SFG-Δmyr.iMC.2A-CD19 (pBP0606) and SFG-FRB_{L}2. Caspase9.2A-Q.8stm.zeta (pBP0668). Transduced T cells were then plated with 5-fold dilutions of rapamycin. After 24 hours, cells were harvested and analyzed by flow cytometry for expression of iMC (via anti-CD19-APC), Caspase-9 (via anti-CD34-PE), and T cell identity (via anti-CD3-PerCPCy5.5). Cells were initially gated for lymphocyte morphology by FSC vs SSC, followed by CD3 expression (˜99% of lymphocytes).

To focus on doubly transduced cells, CD3⁺ lymphocytes were gated on CD19⁺ (ΔMyr.iMC.2A-CD19) and CD34⁺ (FRBₗ2. Caspase9.2A-Q.8stm.zeta) expression. To normalize gated populations, percentages of CD34⁺CD19⁺ cells were divided by percent CD19⁺CD34⁻ cells within each sample as an internal control. Those values were then normalized to drug-free wells for each transduction, which were set at 100%. The results show rapid and efficient elimination of doubly transduced cells in the presence of relatively low (2 nM) levels of rapamycin (FIG. 17A, C). Similar analysis was applied to the Hi-, Med-, and Lo-expressing cells within the CD34⁺CD19⁺ gate (FIG. 17B). As rapamycin concentrations increase, percentage of CD34⁺CD19⁺ cells decrease, indicating elimination of cells. Finally, T cells from a single donor were transduced with ΔMyr.iMC.2A-CD19 (pBP0606) and FRB_{L}2. Caspase9.2A-Q.8stm.zeta (pBP0668) and plated in IL-2-containing media along with varying concentrations of rapamycin for 24 or 48 hrs. After 24 or 48 hrs, cells were harvested and analyzed by flow, as above. Interestingly, although elimination of cells expressing high levels of both transgenes was nearly complete at 24 hours, by 48 hours even cells expressing low levels of both transgenes are killed by rapamycin, showing the efficiency of the process in primary T cells (FIG. 17D).

### Example 23: Examples of Plasmids and Sequences Discussed in Examples 17-21

pBP0044: pSH1-iCaspase9 wt

| | | SEQ | | SEQ ID |
|---|---|---|---|---|
| Fragment | Nucleotide | ID NO: | Peptide | NO: |
| Linker | ATG-CTCGAG | 517 | MLE | 518 |
| FKBPv36 | GGAGTGCAGGTGGAgACtATC | 519 | GVQVETISPGDGRTFPKRGQTCVVH | 520 |
| | TCCCCAGGAGACGGGCGCAC | | YTGMLEDGKKVDSSRDRNKPFKFML | |
| | CTTCCCCAAGCGCGGCCAGA | | GKQEVIRGWEEGVAQMSVGQRAKL | |
| | CCTGCGTGGTGCACTACACC | | TISPDYAYGATGHPGIIPPHATLVFDV | |
| | GGGATGCTTGAAGATGGAAA | | ELLKL | |
| | GAAAGTTGATTCCTCCCGGGA | | | |
| | CAGAAACAAGCCCTTTAAGTT | | | |
| | TATGCTAGGCAAGCAGGAGG | | | |
| | TGATCCGAGGCTGGGAAGAA | | | |
| | GGGGTTGCCCAGATGAGTGT | | | |
| | GGGTCAGAGAGCCAAACTGA | | | |
| | CTATATCTCCAGATTATGCCT | | | |
| | ATGGTGCCACTGGGCACCCA | | | |
| | GGCATCATCCCACCACATGC | | | |
| | CACTCTCGTCTTCGATGTGGA | | | |
| | GCTTCTAAAACTGGA | | | |
| Linker | ATCTGGCGGTGGATCCGGA | 521 | SGGGSG | 522 |
| ΔCaspase9 | GTCGACGGATTTGGTGATGTC | 523 | VDGFGDVGALESLRGNADLAYILSM | 524 |
| | GGTGCTCTTGAGAGTTTGAG | | EPCGHCLIINNVNFCRESGLRTRTGS | |
| | GGGAAATGCAGATTTGGCTTA | | NIDCEKLRRRFSSLHFMVEVKGDLTA | |
| | CATCCTGAGCATGGAGCCCT | | KKMVLALLELARQDHGALDCCVVVIL | |
| | GTGGCCACTGCCTCATTATCA | | SHGCQASHLQFPGAVYGTDGCPVS | |
| | ACAATGTGAACTTCTGCCGTG | | VEKIVNIFNGTSCPSLGGKPKLFFIQA | |
| | AGTCCGGGCTCCGCACCCGC | | CGGEQKDHGFEVASTSPEDESPGS | |
| | ACTGGCTCCAACATCGACTGT | | NPEPDATPFQEGLRTFDQLDAISSLP | |
| | GAGAAGTTGCGGCGTCGCTT | | TPSDIFVSYSTFPGFVSWRDPKSGS | |
| | CTCCTCGCTGCATTTCATGGT | | WYVETLDDIFEQWAHSEDLQSLLLR | |
| | GGAGGTGAAGGGCGACCTGA | | VANAVSVKGIYKQMPGCFNFLRKKL | |
| | CTGCCAAGAAAATGGTGCTG | | FFKTS | |
| | GCTTTGCTGGAGCTGGCGCg | | | |
| | GCAGGACCACGGTGCTCTGG | | | |
| | ACTGCTGCGTGGTGGTCATTC | | | |
| | TCTCTCACGGCTGTCAGGCC | | | |
| | AGCCACCTGCAGTTCCCAGG | | | |
| | GGCTGTCTACGGCACAGATG | | | |
| | GATGCCCTGTGTCGGTCGAG | | | |
| | AAGATTGTGAACATCTTCAAT | | | |
| | GGGACCAGCTGCCCCAGCCT | | | |
| | GGGAGGGAAGCCCAAGCTCT | | | |
| | TTTTCATCCAGGCCTGTGGTG | | | |
| | GGGAGCAGAAAGACCATGGG | | | |
| | TTTGAGGTGGCCTCCACTTCC | | | |
| | CCTGAAGACGAGTCCCCTGG | | | |
| | CAGTAACCCCGAGCCAGATG | | | |
| | CCACCCCGTTCCAGGAAGGT | | | |
| | TTGAGGACCTTCGACCAGCT | | | |
| | GGACGCCATATCTAGTTTGCC | | | |
| | CACACCCAGTGACATCTTTGT | | | |
| | GTCCTACTCTACTTTCCCAGG | | | |
| | TTTTGTTTCCTGGAGGGACCC | | | |
| | CAAGAGTGGCTCCTGGTACG | | | |
| | TTGAGACCCTGGACGACATCT | | | |
| | TTGAGCAGTGGGCTCACTCT | | | |
| | GAAGACCTGCAGTCCCTCCT | | | |
| | GCTTAGGGTCGCTAATGCTGT | | | |
| | TTCGGTGAAAGGGATTTATAA | | | |
| | ACAGATGCCTGGTTGCTTTAA | | | |
| | TTTCCTCCGGAAAAAACTTTT | | | |
| | CTTTAAAACATCAGCTAGCAG | | | |
| | AGCCGAGGGCAGGGGAAGTC | | | |
| | TTCTAACATGCGGGGACGTG | | | |
| | GAGGAAAATCCCGGGCCC-tga | | | |
| Linker | GCTAGCAGAGCC | 525 | ASRA | 526 |
| T2A | GAGGGCAGGGGAAGTCTTCT | 527 | EGRGSLLTCGDVEENPGP* | 528 |
| | AACATGCGGGGACGTGGAGG | | | |
| | AAAATCCCGGGCCC-tga | | | |

pBP0463-pSH1-Fpk-Fpk′.LS.Fpk″.Fpk′″.LS.HA

| | | SEQ | | SEQ ID |
|---|---|---|---|---|
| Fragment | Nucleotide | ID NO: | Peptide | NO: |
| Linker | ATGCTCGAG | 529 | MLE | 530 |
| FRBI | TGGCATGAAGGGTTGGAAGA | 531 | GVQVETISPGDGRTFPKRGQTCVVH | 532 |
| | AGCTTCAAGGCTGTACTTCGG | | YTGMLEDGKKFDSSRDRNKPFKFML | |
| | AGAGAGGAACGTGAAGGGCA | | GKQEVIRGWEEGVAQMSVGQRAKL | |
| | TGTTTGAGGTTCTTGAACCTC | | TISPDYAYGATGHPPKIPPHATLVFD | |
| | TGCACGCCATGATGGAACGG | | VELLKLE | |
| | GGACCGCAGACACTGAAAGA | | | |
| | AACCTCTTTTAATCAGGCCTA | | | |
| | CGGCAGAGACCTGATGGAGG | | | |
| | CCCAAGAATGGTGTAGAAAGT | | | |
| | ATATGAAATCCGGTAACGTGA | | | |
| | AAGACCTGCTCCAGGCCTGG | | | |
| | GACCTTTATTACCATGTGTTC | | | |
| | AGGCGGATCAGTAAG | | | |
| Linker | TCAGGCGGTGGCTCAGGTGT | 533 | SGGGSGVD | 534 |
| | CGAG | | | |
| Δ-Caspase9 | GTCGACGGATTTGGTGATGTC | 535 | DGFGDVGALESLRGNADLAYILSME | 536 |
| | GGTGCTCTTGAGAGTTTGAG | | PCGHCLIINNVNFCRESGLRTRTGSN | |
| | GGGAAATGCAGATTTGGCTTA | | IDCEKLRRRFSSLHFMVEVKGDLTAK | |
| | CATCCTGAGCATGGAGCCCT | | KMVLALLELARQDHGALDCCVVVILS | |
| | GTGGCCACTGCCTCATTATCA | | HGCQASHLQFPGAVYGTDGCPVSV | |
| | ACAATGTGAACTTCTGCCGTG | | EKIVNIFNGTSCPSLGGKPKLFFIQAC | |
| | AGTCCGGGCTCCGCACCCGC | | GGEQKDHGFEVASTSPEDESPGSN | |
| | ACTGGCTCCAACATCGACTGT | | PEPDATPFQEGLRTFDQLDAISSLPT | |
| | GAGAAGTTGCGGCGTCGCTT | | PSDIFVSYSTFPGFVSWRDPKSGSW | |
| | CTCCTCGCTGCATTTCATGGT | | YVETLDDIFEQWAHSEDLQSLLLRVA | |
| | GGAGGTGAAGGGCGACCTGA | | NAVSVKGIYKQMPGCFNFLRKKLFF | |
| | CTGCCAAGAAAATGGTGCTG | | KTSASRA | |
| | GCTTTGCTGGAGCTGGCGCg | | | |
| | GCAGGACCACGGTGCTCTGG | | | |
| | ACTGCTGCGTGGTGGTCATTC | | | |
| | TCTCTCACGGCTGTCAGGCC | | | |
| | AGCCACCTGCAGTTCCCAGG | | | |
| | GGCTGTCTACGGCACAGATG | | | |
| | GATGCCCTGTGTCGGTCGAG | | | |
| | AAGATTGTGAACATCTTCAAT | | | |
| | GGGACCAGCTGCCCCAGCCT | | | |
| | GGGAGGGAAGCCCAAGCTCT | | | |
| | TTTTCATCCAGGCCTGTGGTG | | | |
| | GGGAGCAGAAAGACCATGGG | | | |
| | TTTGAGGTGGCCTCCACTTCC | | | |
| | CCTGAAGACGAGTCCCCTGG | | | |
| | CAGTAACCCCGAGCCAGATG | | | |
| | CCACCCCGTTCCAGGAAGGT | | | |
| | TTGAGGACCTTCGACCAGCT | | | |
| | GGACGCCATATCTAGTTTGCC | | | |
| | CACACCCAGTGACATCTTTGT | | | |
| | GTCCTACTCTACTTTCCCAGG | | | |
| | TTTTGTTTCCTGGAGGGACCC | | | |
| | CAAGAGTGGCTCCTGGTACG | | | |
| | TTGAGACCCTGGACGACATCT | | | |
| | TTGAGCAGTGGGCTCACTCT | | | |
| | GAAGACCTGCAGTCCCTCCT | | | |
| | GCTTAGGGTCGCTAATGCTGT | | | |
| | TTCGGTGAAAGGGATTTATAA | | | |
| | ACAGATGCCTGGTTGCTTTAA | | | |
| | TTTCCTCCGGAAAAAACTTTT | | | |
| | CTTTAAAACATCAGCTAGCAG | | | |
| | AGCC | | | |
| T2A | GAGGGCAGGGGAAGTCTTCT | 537 | EGRGSLLTCGDVEENPGP | 538 |
| | AACATGCGGGGACGTGGAGG | | | |
| | AAAATCCCGGGCCCtga | | | |

pBP0725-pSH1-FRBI.FRBI′.LS.FRBI″.FRBI′″

| | | SEQ ID | | SEQ |
|---|---|---|---|---|
| Fragment | Nucleotide | NO: | Peptide | ID NO: |
| FRBI | ATGctcgagTGGCATGAAGGCC | 539 | MLEWHEGLEEASRLYFGERNVKGM | 540 |
| | TGGAAGAGGCATCTCGTTTGT | | FEVLEPLHAMMERGPQTLKETSFNQ | |
| | ACTTTGGGGAAAGGAACGTG | | AYGRDLMEAQEWCRKYMKSGNVKD | |
| | AAAGGCATGTTTGAGGTGCTG | | LLQAWDLYYHVFRRISK | |
| | GAGCCCTTGCACGCTATGATG | | | |
| | GAACGGGGCCCCCAGACTCT | | | |
| | GAAGGAAACATCCTTTAATCA | | | |
| | GGCCTATGGTCGAGATTTAAT | | | |
| | GGAGGCCCAAGAGTGGTGCA | | | |
| | GGAAGTACATGAAATCAGGG | | | |
| | AATGTCAAGGACCTCCTCCAA | | | |
| | GCCTGGGACCTCTATTATCAT | | | |
| | GTGTTCCGACGAATCTCAAAG | | | |
| Linker | gtcgag | 541 | VD | 542 |
| FRBI′ | TGGCATGAAGGGTTGGAAGA | 543 | WHEGLEEASRLYFGERNVKGMFEV | 544 |
| | AGCTTCAAGGCTGTACTTCGG | | LEPLHAMMERGPQTLKETSFNQAYG | |
| | AGAGAGGAACGTGAAGGGCA | | RDLMEAQEWCRKYMKSGNVKDLLQ | |
| | TGTTTGAGGTTCTTGAACCTC | | AWDLYYHVFRRISK | |
| | TGCACGCCATGATGGAACGG | | | |
| | GGACCGCAGACACTGAAAGA | | | |
| | AACCTCTTTTAATCAGGCCTA | | | |
| | CGGCAGAGACCTGATGGAGG | | | |
| | CCCAAGAATGGTGTAGAAAGT | | | |
| | ATATGAAATCCGGTAACGTGA | | | |
| | AAGACCTGCTCCAGGCCTGG | | | |
| | GACCTTTATTACCATGTGTTC | | | |
| | AGGCGGATCAGTAAG | | | |
| Linker | TCAGGCGGTGGCTCAGGTGT | 545 | SGGGSGVD | 546 |
| | CGAG | | | |
| FRBI″ | TGGCATGAAGGCCTGGAAGA | 547 | WHEGLEEASRLYFGERNVKGMFEV | 548 |
| | GGCATCTCGTTTGTACTTTGG | | LEPLHAMMERGPQTLKETSFNQAYG | |
| | GGAAAGGAACGTGAAAGGCA | | RDLMEAQEWCRKYMKSGNVKDLLQ | |
| | TGTTTGAGGTGCTGGAGCCCT | | AWDLYYHVFRRISK | |
| | TGCACGCTATGATGGAACGG | | | |
| | GGCCCCCAGACTCTGAAGGA | | | |
| | AACATCCTTTAATCAGgCCTAT | | | |
| | GGTCGAGATTTAATGGAGGC | | | |
| | CCAAGAGTGGtGCAGGAAGTA | | | |
| | CATGAAATCAGGGAATGTCAA | | | |
| | GGACCTCCTCCAAGCCTGGG | | | |
| | ACCTCTATTATCATGTGTTCC | | | |
| | GACGAATCTCAAAG | | | |
| Linker | GTCGAC | 549 | VD | 550 |
| FRBI′′′ | TGGCATGAAGGGTTGGAAGA | 551 | WHEGLEEASRLYFGERNVKGMFEV | 552 |
| | AGCTTCAAGGCTGTACTTCGG | | LEPLHAMMERGPQTLKETSFNQAYG | |
| | AGAGAGGAACGTGAAGGGCA | | RDLMEAQEWCRKYMKSGNVKDLLQ | |
| | TGTTTGAGGTTCTTGAACCTC | | AWDLYYHVFRRISK | |
| | TGCACGCCATGATGGAACGG | | | |
| | GGACCGCAGACACTGAAAGA | | | |
| | AACCTCTTTTAATCAGGCCTA | | | |
| | CGGCAGAGACCTGATGGAGG | | | |
| | CCCAAGAATGGTGTaGAAAGT | | | |
| | ATATGAAATCCGGTAACGTGA | | | |
| | AAGACCTGCTCCAGGCCTGG | | | |
| | GACCTTTATTACCATGTGTTC | | | |
| | AGGCGGATCAGTAAGTCAGG | | | |
| | CGGTGGCTCAGGTGTCGAC | | | |
| Linker | GTCGAC | 553 | VE | 554 |
| HA tag | TATCCGTACGACGTACCAGAC | 555 | YPYDVPDYALD* | 556 |
| | TACGCACTCGACTAA | | | |

pBP0465-pSH1-M-FRBI.FRBI′.LS.HA

| | | SEQ | | SEQ |
|---|---|---|---|---|
| | | ID | | ID |
| Fragment | Nucleotide | NO: | Peptide | NO: |
| Myr | atgggctgtgtgcaatgtaaggataaagaa | 557 | MGCVQCKDKEATKLTEE | 558 |
| | gcaacaaaactgacggaggag | | | |
| Linker | CTCGAG | 559 | LG | 560 |
| FRBI | TGGCATGAAGGCCTGGAAGA | 561 | MLEWHEGLEEASRLYFGERNVKGM | 562 |
| | GGCATCTCGTTTGTACTTTGG | | FEVLEPLHAMMERGPQTLKETSFNQ | |
| | GGAAAGGAACGTGAAAGGCA | | AYGRDLMEAQEWCRKYMKSGNVKD | |
| | TGTTTGAGGTGCTGGAGCCCT | | LLQAVVDLYYHVFRRISK | |
| | TGCACGCTATGATGGAACGG | | | |
| | GGCCCCCAGACTCTGAAGGA | | | |
| | AACATCCTTTAATCAGGCCTA | | | |
| | TGGTCGAGATTTAATGGAGGC | | | |
| | CCAAGAGTGGTGCAGGAAGT | | | |
| | ACATGAAATCAGGGAATGTCA | | | |
| | AGGACCTCCTCCAAGCCTGG | | | |
| | GACCTCTATTATCATGTGTTC | | | |
| | CGACGAATCTCAAAG | | | |
| Linker | gtcgag | 563 | VD | 564 |
| FRBI′ | TGGCATGAAGGGTTGGAAGA | 565 | WHEGLEEASRLYFGERNVKGMFEV | 566 |
| | AGCTTCAAGGCTGTACTTCGG | | LEPLHAMMERGPQTLKETSFNQAYG | |
| | AGAGAGGAACGTGAAGGGCA | | RDLMEAQEWCRKYMKSGNVKDLLQ | |
| | TGTTTGAGGTTCTTGAACCTC | | AWDLYYHVFRRISK | |
| | TGCACGCCATGATGGAACGG | | | |
| | GGACCGCAGACACTGAAAGA | | | |
| | AACCTCTTTTAATCAGGCCTA | | | |
| | CGGCAGAGACCTGATGGAGG | | | |
| | CCCAAGAATGGTGTAGAAAGT | | | |
| | ATATGAAATCCGGTAACGTGA | | | |
| | AAGACCTGCTCCAGGCCTGG | | | |
| | GACCTTTATTACCATGTGTTC | | | |
| | AGGCGGATCAGTAAG | | | |
| Linker | TCAGGCGGTGGCTCAGGTG | 567 | SGGGSGVD | 568 |
| HA tag | tatccgtacgacgtaccagactacgcactc | 569 | YPYDVPDYALD* | 570 |
| | gactaa | | | |

pBP0722-pSH1-Fpk-Fpk′.LS.Fpk″.Fpk′″.LS.HA

| | | SEQ | | SEQ |
|---|---|---|---|---|
| | | ID | | ID |
| Fragment | Nucleotide | NO: | Peptide | NO: |
| Linker | ATGCTCGAG | 571 | MLE | 572 |
| FKBPpk | GGcGTcCAaGTcGAaACcATtagt | 573 | GVQVETISPGDGRTFPKRGQTCVVH | 574 |
| | CCcGGcGAtGGcaGaACaTTtCC | | YTGMLEDGKKFDSSRDRNKPFKFML | |
| | tAAaaGgGGaCAaACaTGtGTcG | | GKQEVIRGWEEGVAQMSVGQRAKL | |
| | TcCAtTAtACaGGcATGtTgGAgG | | TISPDYAYGATGHPPKIPPHATLVFD | |
| | AcGGcAAaAAgttcGAcagtagtaGa | | VELLKLE | |
| | GAtcGcAAtAAaCCtTTcAAaTTc | | | |
| | ATGtTgGGaAAaCAaGAaGTcAT | | | |
| | taGgGGaTGGGAgGAgGGcGTg | | | |
| | GCtCAaATGtccGTcGGcCAacG | | | |
| | cGCtAAgCTcACcATcagcCCcGA | | | |
| | cTAcGCaTAcGGcGCtACcGGaC | | | |
| | AtCCccctaagATtCCcCCtCAcGC | | | |
| | tACctTgGTgTTtGAcGTcGAaCT | | | |
| | gtTgAAgCTcGAa | | | |
| Linker | gtcgag | 575 | VD | 576 |
| FKBPpk′ | ggagtgcaggtggagactatctccccagg | 577 | GVQVETISPGDGRTFPKRGQTCVVH | 578 |
| | agacgggcgcaccttccccaagcgcggc | | YTGMLEDGKKFDSSRDRNKPFKFML | |
| | cagacctgcgtggtgcactacaccgggat | | GKQEVIRGWEEGVAQMSVGQRAKL | |
| | gcttgaagatggaaagaaattcgattcctct | | TISPDYAYGATGHPPKIPPHATLVFD | |
| | cgggacagaaacaagccctttaagtttatg | | VELLKLE | |
| | ctaggcaagcaggaggtgatccgaggctg | | | |
| | ggaagaaggggttgcccagatgagtgtgg | | | |
| | gtcagagagccaaactgactatatctccag | | | |
| | attatgcctatggtgccactgggcacccacc | | | |
| | taagatcccaccacatgccactctcgtcttc | | | |
| | gatgtggagcttctaaaactggaa | | | |
| Linker | TCAGGCGGTGGCTCAGGTGT | 579 | SGGGSGVD | 580 |
| | CGAG | | | |
| FKBPpk″ | GGcGTcCAaGTcGAaACcATtagt | 581 | GVQVETISPGDGRTFPKRGQTCVVH | 582 |
| | CCcGGcGAtGGcaGaACaTTtCC | | YTGMLEDGKKFDSSRDRNKPFKFML | |
| | tAAaaGgGGaCAaACaTGtGTcG | | GKQEVIRGWEEGVAQMSVGQRAKL | |
| | TcCAtTAtACaGGcATGtTgGAgG | | TISPDYAYGATGHPPKIPPHATLVFD | |
| | AcGGcAAaAAgttcGAcagtagtaGa | | VELLKLE | |
| | GAtcGcAAtAAaCCtTTcAAaTTc | | | |
| | ATGtTgGGaAAaCAaGAaGTcAT | | | |
| | taGgGGaTGGGAgGAgGGcGTg | | | |
| | GCtCAaATGtccGTcGGcCAacG | | | |
| | cGCtAAgCTcACcATcagcCCcGA | | | |
| | cTAcGCaTAcGGcGCtACcGGaC | | | |
| | AtCCccctaagATtCCcCCtCAcGC | | | |
| | tACctTgGTgTTtGAcGTcGAaCT | | | |
| | gtTgAAgCTcGAa | | | |
| Linker | GTCGAC | 583 | VD | 584 |
| FKBPpk′′′ | ggagtgcaggtggagactatctccccagg | 585 | GVQVETISPGDGRTFPKRGQTCVVH | 586 |
| | agacgggcgcaccttccccaagcgcggc | | YTGMLEDGKKFDSSRDRNKPFKFML | |
| | cagacctgcgtggtgcactacaccgggat | | GKQEVIRGWEEGVAQMSVGQRAKL | |
| | gcttgaagatggaaagaaattcgattcctct | | TISPDYAYGATGHPPKIPPHATLVFD | |
| | cgggacagaaacaagccctttaagtttatg | | VELLKLE | |
| | ctaggcaagcaggaggtgatccgaggctg | | | |
| | ggaagaaggggttgcccagatgagtgtgg | | | |
| | gtcagagagccaaactgactatatctccag | | | |
| | attatgcctatggtgccactgggcacccacc | | | |
| | taagatcccaccacatgccactctcgtcttc | | | |
| | gatgtggagcttctaaaactggaa | | | |
| Linker | TCAGGCGGTGGCTCAGGTGT | 587 | SGGGSGVD | 588 |
| | CGAG | | | |
| HA tag | TATCCGTACGACGTACCAGAC | 589 | YPYDVPDYALD* | 590 |
| | TACGCACTCGACTAA | | | |

pBP0220-pSFG-iC9.T2A-ΔCD19

| | | SEQ | | SEQ |
|---|---|---|---|---|
| | | ID | | ID |
| Fragment | Nucleotide | NO: | Peptide | NO: |
| FKBP12v36 | ATGCTCGAGGGAGTGCAGGTG | 591 | MLEGVQVETISPGDGRTFPKRGQ | 592 |
| | GAGACTATCTCCCCAGGAGAC | | TCVVHYTGMLEDGKKVDSSRDRN | |
| | GGGCGCACCTTCCCCAAGCGC | | KPFKFMLGKQEVIRGWEEGVAQM | |
| | GGCCAGACCTGCGTGGTGCAC | | SVGQRAKLTISPDYAYGATGHPGII | |
| | TACACCGGGATGCTTGAAGAT | | PPHATLVFDVELLKLE | |
| | GGAAAGAAAGTTGATTCCTCC | | | |
| | CGGGACAGAAACAAGCCCTTT | | | |
| | AAGTTTATGCTAGGCAAGCAG | | | |
| | GAGGTGATCCGAGGCTGGGAA | | | |
| | GAAGGGGTTGCCCAGATGAGT | | | |
| | GTGGGTCAGAGAGCCAAACTG | | | |
| | ACTATATCTCCAGATTATGCCT | | | |
| | ATGGTGCCACTGGGCACCCAG | | | |
| | GCATCATCCCACCACATGCCA | | | |
| | CTCTCGTCTTCGATGTGGAGCT | | | |
| | TCTAAAACTGGAA | | | |
| Linker | TCTGGCGGTGGATCCGGA | 593 | SGGGSG | 594 |
| ΔCaspase9 | GTCGACGGATTTGGTGATGTC | 595 | VDGFGDVGALESLRGNADLAYILS | 596 |
| | GGTGCTCTTGAGAGTTTGAGG | | MEPCGHCLIINNVNFCRESGLRTR | |
| | GGAAATGCAGATTTGGCTTACA | | TGSNIDCEKLRRRFSSLHFMVEVK | |
| | TCCTGAGCATGGAGCCCTGTG | | GDLTAKKMVLALLELARQDHGALD | |
| | GCCACTGCCTCATTATCAACAA | | CCVVVILSHGCQASHLQFPGAVYG | |
| | TGTGAACTTCTGCCGTGAGTC | | TDGCPVSVEKIVNIFNGTSCPSLG | |
| | CGGGCTCCGCACCCGCACTGG | | GKPKLFFIQACGGEQKDHGFEVAS | |
| | CTCCAACATCGACTGTGAGAA | | TSPEDESPGSNPEPDATPFQEGL | |
| | GTTGCGGCGTCGCTTCTCCTC | | RTFDQLDAISSLPTPSDIFVSYSTF | |
| | GCTGCATTTCATGGTGGAGGT | | PGFVSWRDPKSGSWYVETLDDIF | |
| | GAAGGGCGACCTGACTGCCAA | | EQWAHSEDLQSLLLRVANAVSVK | |
| | GAAAATGGTGCTGGCTTTGCT | | GIYKQMPGCFNFLRKKLFFKTSAS | |
| | GGAGCTGGCGCGGCAGGACC | | RA | |
| | ACGGTGCTCTGGACTGCTGCG | | | |
| | TGGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCC | | | |
| T2A | GAGGGCAGGGGAAGTCTTCTA | 597 | EGRGSLLTCGDVEENPGP | 598 |
| | ACATGCGGGGACGTGGAGGAA | | | |
| | AATCCCGGGCCC | | | |
| ΔCD19 | ATGCCACCTCCTCGCCTCCTCT | 599 | MPPPRLLFFLLFLTPMEVRPEEPL | 600 |
| | TCTTCCTCCTCTTCCTCACCCC | | VVKVEEGDNAVLQCLKGTSDGPT | |
| | CATGGAAGTCAGGCCCGAGGA | | QQLTWSRESPLKPFLKLSLGLPGL | |
| | ACCTCTAGTGGTGAAGGTGGA | | GIHMRPLAIWLFIFNVSQQMGGFY | |
| | AGAGGGAGATAACGCTGTGCT | | LCQPGPPSEKAWQPGWTVNVEG | |
| | GCAGTGCCTCAAGGGGACCTC | | SGELFRWNVSDLGGLGCGLKNRS | |
| | AGATGGCCCCACTCAGCAGCT | | SEGPSSPSGKLMSPKLYVWAKDR | |
| | GACCTGGTCTCGGGAGTCCCC | | PEIWEGEPPCLPPRDSLNQSLSQD | |
| | GCTTAAACCCTTCTTAAAACTC | | LTMAPGSTLWLSCGVPPDSVSRG | |
| | AGCCTGGGGCTGCCAGGCCTG | | PLSWTHVHPKGPKSLLSLELKDDR | |
| | GGAATCCACATGAGGCCCCTG | | PARDMWVMETGLLLPRATAQDAG | |
| | GCCATCTGGCTTTTCATCTTCA | | KYYCHRGNLTMSFHLEITARPVLW | |
| | ACGTCTCTCAACAGATGGGGG | | HWLLRTGGWKVSAVTLAYLIFCLC | |
| | GCTTCTACCTGTGCCAGCCGG | | SLVGILHLQRALVLRRKRKRMTDP | |
| | GGCCCCCCTCTGAGAAGGCCT | | TRRF* | |
| | GGCAGCCTGGCTGGACAGTCA | | | |
| | ATGTGGAGGGCAGCGGGGAG | | | |
| | CTGTTCCGGTGGAATGTTTCG | | | |
| | GACCTAGGTGGCCTGGGCTGT | | | |
| | GGCCTGAAGAACAGGTCCTCA | | | |
| | GAGGGCCCCAGCTCCCCTTCC | | | |
| | GGGAAGCTCATGAGCCCCAAG | | | |
| | CTGTATGTGTGGGCCAAAGAC | | | |
| | CGCCCTGAGATCTGGGAGGGA | | | |
| | GAGCCTCCGTGTCTCCCACCG | | | |
| | AGGGACAGCCTGAACCAGAGC | | | |
| | CTCAGCCAGGACCTCACCATG | | | |
| | GCCCCTGGCTCCACACTCTGG | | | |
| | CTGTCCTGTGGGGTACCCCCT | | | |
| | GACTCTGTGTCCAGGGGCCCC | | | |
| | CTCTCCTGGACCCATGTGCAC | | | |
| | CCCAAGGGGCCTAAGTCATTG | | | |
| | CTGAGCCTAGAGCTGAAGGAC | | | |
| | GATCGCCCGGCCAGAGATATG | | | |
| | TGGGTAATGGAGACGGGTCTG | | | |
| | TTGTTGCCCCGGGCCACAGCT | | | |
| | CAAGACGCTGGAAAGTATTATT | | | |
| | GTCACCGTGGCAACCTGACCA | | | |
| | TGTCATTCCACCTGGAGATCAC | | | |
| | TGCTCGGCCAGTACTATGGCA | | | |
| | CTGGCTGCTGAGGACTGGTGG | | | |
| | CTGGAAGGTCTCAGCTGTGAC | | | |
| | TTTGGCTTATCTGATCTTCTGC | | | |
| | CTGTGTTCCCTTGTGGGCATTC | | | |
| | TTCATCTTCAAAGAGCCCTGGT | | | |
| | CCTGAGGAGGAAAAGAAAGCG | | | |
| | AATGACTGACCCCACCAGGAG | | | |
| | ATTCTAA | | | |

pBP0756-pSFG-iC9.T2A-dCD19.P2A-FRBₗ

| | | SEQ ID | | SEQ |
|---|---|---|---|---|
| Fragment | Nucleotide | NO: | Peptide | ID NO: |
| FKBP12v36 | ATGCTCGAGGGAGTGCAGGTG | 601 | MLEGVQVETISPGDGRTFPKRGQ | 602 |
| | GAGACTATCTCCCCAGGAGAC | | TCVVHYTGMLEDGKKVDSSRDRN | |
| | GGGCGCACCTTCCCCAAGCGC | | KPFKFMLGKQEVIRGWEEGVAQM | |
| | GGCCAGACCTGCGTGGTGCAC | | SVGQRAKLTISPDYAYGATGHPGII | |
| | TACACCGGGATGCTTGAAGAT | | PPHATLVFDVELLKLE | |
| | GGAAAGAAAGTTGATTCCTCC | | | |
| | CGGGACAGAAACAAGCCCTTT | | | |
| | AAGTTTATGCTAGGCAAGCAG | | | |
| | GAGGTGATCCGAGGCTGGGAA | | | |
| | GAAGGGGTTGCCCAGATGAGT | | | |
| | GTGGGTCAGAGAGCCAAACTG | | | |
| | ACTATATCTCCAGATTATGCCT | | | |
| | ATGGTGCCACTGGGCACCCAG | | | |
| | GCATCATCCCACCACATGCCA | | | |
| | CTCTCGTCTTCGATGTGGAGCT | | | |
| | TCTAAAACTGGAA | | | |
| Linker | TCTGGCGGTGGATCCGGA | 603 | SGGGSG | 604 |
| dCaspase9 | GTCGACGGATTTGGTGATGTC | 605 | VDGFGDVGALESLRGNADLAYILS | 606 |
| | GGTGCTCTTGAGAGTTTGAGG | | MEPCGHCLIINNVNFCRESGLRTR | |
| | GGAAATGCAGATTTGGCTTACA | | TGSNIDCEKLRRRFSSLHFMVEVK | |
| | TCCTGAGCATGGAGCCCTGTG | | GDLTAKKMVLALLELARQDHGALD | |
| | GCCACTGCCTCATTATCAACAA | | CCVVVILSHGCQASHLQFPGAVYG | |
| | TGTGAACTTCTGCCGTGAGTC | | TDGCPVSVEKIVNIFNGTSCPSLG | |
| | CGGGCTCCGCACCCGCACTGG | | GKPKLFFIQACGGEQKDHGFEVAS | |
| | CTCCAACATCGACTGTGAGAA | | TSPEDESPGSNPEPDATPFQEGL | |
| | GTTGCGGCGTCGCTTCTCCTC | | RTFDQLDAISSLPTPSDIFVSYSTF | |
| | GCTGCATTTCATGGTGGAGGT | | PGFVSWRDPKSGSWYVETLDDIF | |
| | GAAGGGCGACCTGACTGCCAA | | EQWAHSEDLQSLLLRVANAVSVK | |
| | GAAAATGGTGCTGGCTTTGCT | | GIYKQMPGCFNFLRKKLFFKTSAS | |
| | GGAGCTGGCGCGGCAGGACC | | RA | |
| | ACGGTGCTCTGGACTGCTGCG | | | |
| | TGGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCC | | | |
| T2A | GAGGGCAGGGGAAGTCTTCTA | 607 | EGRGSLLTCGDVEENPGP | 608 |
| | ACATGCGGGGACGTGGAGGAA | | | |
| | AATCCCGGGCCC | | | |
| dCD19 | ATGCCACCTCCTCGCCTCCTCT | 609 | MPPPRLLFFLLFLTPMEVRPEEPL | 610 |
| | TCTTCCTCCTCTTCCTCACCCC | | VVKVEEGDNAVLQCLKGTSDGPT | |
| | CATGGAAGTCAGGCCCGAGGA | | QQLTWSRESPLKPFLKLSLGLPGL | |
| | ACCTCTAGTGGTGAAGGTGGA | | GIHMRPLAIWLFIFNVSQQMGGFY | |
| | AGAGGGAGATAACGCTGTGCT | | LCQPGPPSEKAWQPGWTVNVEG | |
| | GCAGTGCCTCAAGGGGACCTC | | SGELFRWNVSDLGGLGCGLKNRS | |
| | AGATGGCCCCACTCAGCAGCT | | SEGPSSPSGKLMSPKLYVWAKDR | |
| | GACCTGGTCTCGGGAGTCCCC | | PEIWEGEPPCLPPRDSLNQSLSQD | |
| | GCTTAAACCCTTCTTAAAACTC | | LTMAPGSTLWLSCGVPPDSVSRG | |
| | AGCCTGGGGCTGCCAGGCCTG | | PLSWTHVHPKGPKSLLSLELKDDR | |
| | GGAATCCACATGAGGCCCCTG | | PARDMWVMETGLLLPRATAQDAG | |
| | GCCATCTGGCTTTTCATCTTCA | | KYYCHRGNLTMSFHLEITARPVLW | |
| | ACGTCTCTCAACAGATGGGGG | | HWLLRTGGWKVSAVTLAYLIFCLC | |
| | GCTTCTACCTGTGCCAGCCGG | | SLVGILHLQRALVLRRKRKRMTDP | |
| | GGCCCCCCTCTGAGAAGGCCT | | TRRF | |
| | GGCAGCCTGGCTGGACAGTCA | | | |
| | ATGTGGAGGGCAGCGGGGAG | | | |
| | CTGTTCCGGTGGAATGTTTCG | | | |
| | GACCTAGGTGGCCTGGGCTGT | | | |
| | GGCCTGAAGAACAGGTCCTCA | | | |
| | GAGGGCCCCAGCTCCCCTTCC | | | |
| | GGGAAGCTCATGAGCCCCAAG | | | |
| | CTGTATGTGTGGGCCAAAGAC | | | |
| | CGCCCTGAGATCTGGGAGGGA | | | |
| | GAGCCTCCGTGTCTCCCACCG | | | |
| | AGGGACAGCCTGAACCAGAGC | | | |
| | CTCAGCCAGGACCTCACCATG | | | |
| | GCCCCTGGCTCCACACTCTGG | | | |
| | CTGTCCTGTGGGGTACCCCCT | | | |
| | GACTCTGTGTCCAGGGGCCCC | | | |
| | CTCTCCTGGACCCATGTGCAC | | | |
| | CCCAAGGGGCCTAAGTCATTG | | | |
| | CTGAGCCTAGAGCTGAAGGAC | | | |
| | GATCGCCCGGCCAGAGATATG | | | |
| | TGGGTAATGGAGACGGGTCTG | | | |
| | TTGTTGCCCCGGGCCACAGCT | | | |
| | CAAGACGCTGGAAAGTATTATT | | | |
| | GTCACCGTGGCAACCTGACCA | | | |
| | TGTCATTCCACCTGGAGATCAC | | | |
| | TGCTCGGCCAGTACTATGGCA | | | |
| | CTGGCTGCTGAGGACTGGTGG | | | |
| | CTGGAAGGTCTCAGCTGTGAC | | | |
| | TTTGGCTTATCTGATCTTCTGC | | | |
| | CTGTGTTCCCTTGTGGGCATTC | | | |
| | TTCATCTTCAAAGAGCCCTGGT | | | |
| | CCTGAGGAGGAAAAGAAAGCG | | | |
| | AATGACTGACCCCACCAGGAG | | | |
| | ATTC | | | |
| gsg | GGGAGTGGG | 611 | GSG | 612 |
| P2A | GCTACGAATTTTAGCTTGCTGA | 613 | SEQ ID NO: 306 | 614 |
| | AGCAGGCCGGTGATGTGGAAG | | ATNFSLLKQAGDVEENPGP | |
| | AGAACCCCGGGCCT | | | |
| FRBI | TGGCACGAAGGTTTGGAAGAG | 615 | WHEGLEEASRLYFGERNVKGMFE | 616 |
| | GCCTCCCGCCTGTATTTCGGT | | VLEPLHAMMERGPQTLKETSFNQ | |
| | GAGAGAAATGTCAAAGGTATG | | AYGRDLMEAQEWCRKYMKSGNV | |
| | TTTGAAGTGCTTGAGCCCCTG | | KDLLQAWDLYYHVFRRISK* | |
| | CACGCCATGATGGAACGGGGG | | | |
| | CCGCAGACTCTGAAAGAAACC | | | |
| | TCATTCAACCAGGCATACGGG | | | |
| | CGAGACCTGATGGAAGCGCAG | | | |
| | GAATGGTGTAGGAAGTACATG | | | |
| | AAGTCCGGAAATGTGAAGGAC | | | |
| | TTGCTCCAGGCTTGGGACCTG | | | |
| | TACTATCACGTATTTCGGAGAA | | | |
| | TAAGCAAG-TAA | | | |

pBP0755-pSFG-iC9.T2A-dCD19.P2A-FRB2

| | | SEQ ID | | SEQ |
|---|---|---|---|---|
| Fragment | Nucleotide | NO: | Peptide | ID NO: |
| FKBP12v36 | ATGCTCGAGGGAGTGCAGGTG | 617 | MLEGVQVETISPGDGRTFPKRGQ | 618 |
| | GAGACTATCTCCCCAGGAGAC | | TCVVHYTGMLEDGKKVDSSRDRN | |
| | GGGCGCACCTTCCCCAAGCGC | | KPFKFMLGKQEVIRGWEEGVAQM | |
| | GGCCAGACCTGCGTGGTGCAC | | SVGQRAKLTISPDYAYGATGHPGII | |
| | TACACCGGGATGCTTGAAGAT | | PPHATLVFDVELLKLE | |
| | GGAAAGAAAGTTGATTCCTCC | | | |
| | CGGGACAGAAACAAGCCCTTT | | | |
| | AAGTTTATGCTAGGCAAGCAG | | | |
| | GAGGTGATCCGAGGCTGGGAA | | | |
| | GAAGGGGTTGCCCAGATGAGT | | | |
| | GTGGGTCAGAGAGCCAAACTG | | | |
| | ACTATATCTCCAGATTATGCCT | | | |
| | ATGGTGCCACTGGGCACCCAG | | | |
| | GCATCATCCCACCACATGCCA | | | |
| | CTCTCGTCTTCGATGTGGAGCT | | | |
| | TCTAAAACTGGAA | | | |
| Linker | TCTGGCGGTGGATCCGGA | 619 | SGGGSG | 620 |
| ΔCaspase9 | GTCGACGGATTTGGTGATGTC | 621 | VDGFGDVGALESLRGNADLAYILS | 622 |
| | GGTGCTCTTGAGAGTTTGAGG | | MEPCGHCLIINNVNFCRESGLRTR | |
| | GGAAATGCAGATTTGGCTTACA | | TGSNIDCEKLRRRFSSLHFMVEVK | |
| | TCCTGAGCATGGAGCCCTGTG | | GDLTAKKMVLALLELARQDHGALD | |
| | GCCACTGCCTCATTATCAACAA | | CCVVVILSHGCQASHLQFPGAVYG | |
| | TGTGAACTTCTGCCGTGAGTC | | TDGCPVSVEKIVNIFNGTSCPSLG | |
| | CGGGCTCCGCACCCGCACTGG | | GKPKLFFIQACGGEQKDHGFEVAS | |
| | CTCCAACATCGACTGTGAGAA | | TSPEDESPGSNPEPDATPFQEGL | |
| | GTTGCGGCGTCGCTTCTCCTC | | RTFDQLDAISSLPTPSDIFVSYSTF | |
| | GCTGCATTTCATGGTGGAGGT | | PGFVSWRDPKSGSWYVETLDDIF | |
| | GAAGGGCGACCTGACTGCCAA | | EQWAHSEDLQSLLLRVANAVSVK | |
| | GAAAATGGTGCTGGCTTTGCT | | GIYKQMPGCFNFLRKKLFFKTSAS | |
| | GGAGCTGGCGCGGCAGGACC | | RA | |
| | ACGGTGCTCTGGACTGCTGCG | | | |
| | TGGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCC | | | |
| T2A | GAGGGCAGGGGAAGTCTTCTA | 623 | EGRGSLLTCGDVEENPGP | 624 |
| | ACATGCGGGGACGTGGAGGAA | | | |
| | AATCCCGGGCCC | | | |
| ΔCD19 | ATGCCACCTCCTCGCCTCCTCT | 625 | MPPPRLLFFLLFLTPMEVRPEEPL | 626 |
| | TCTTCCTCCTCTTCCTCACCCC | | VVKVEEGDNAVLQCLKGTSDGPT | |
| | CATGGAAGTCAGGCCCGAGGA | | QQLTWSRESPLKPFLKLSLGLPGL | |
| | ACCTCTAGTGGTGAAGGTGGA | | GIHMRPLAIWLFIFNVSQQMGGFY | |
| | AGAGGGAGATAACGCTGTGCT | | LCQPGPPSEKAWQPGWTVNVEG | |
| | GCAGTGCCTCAAGGGGACCTC | | SGELFRWNVSDLGGLGCGLKNRS | |
| | AGATGGCCCCACTCAGCAGCT | | SEGPSSPSGKLMSPKLYVWAKDR | |
| | GACCTGGTCTCGGGAGTCCCC | | PEIWEGEPPCLPPRDSLNQSLSQD | |
| | GCTTAAACCCTTCTTAAAACTC | | LTMAPGSTLWLSCGVPPDSVSRG | |
| | AGCCTGGGGCTGCCAGGCCTG | | PLSWTHVHPKGPKSLLSLELKDDR | |
| | GGAATCCACATGAGGCCCCTG | | PARDMWVMETGLLLPRATAQDAG | |
| | GCCATCTGGCTTTTCATCTTCA | | KYYCHRGNLTMSFHLEITARPVLW | |
| | ACGTCTCTCAACAGATGGGGG | | HWLLRTGGWKVSAVTLAYLIFCLC | |
| | GCTTCTACCTGTGCCAGCCGG | | SLVGILHLQRALVLRRKRKRMTDP | |
| | GGCCCCCCTCTGAGAAGGCCT | | TRRF | |
| | GGCAGCCTGGCTGGACAGTCA | | | |
| | ATGTGGAGGGCAGCGGGGAG | | | |
| | CTGTTCCGGTGGAATGTTTCG | | | |
| | GACCTAGGTGGCCTGGGCTGT | | | |
| | GGCCTGAAGAACAGGTCCTCA | | | |
| | GAGGGCCCCAGCTCCCCTTCC | | | |
| | GGGAAGCTCATGAGCCCCAAG | | | |
| | CTGTATGTGTGGGCCAAAGAC | | | |
| | CGCCCTGAGATCTGGGAGGGA | | | |
| | GAGCCTCCGTGTCTCCCACCG | | | |
| | AGGGACAGCCTGAACCAGAGC | | | |
| | CTCAGCCAGGACCTCACCATG | | | |
| | GCCCCTGGCTCCACACTCTGG | | | |
| | CTGTCCTGTGGGGTACCCCCT | | | |
| | GACTCTGTGTCCAGGGGCCCC | | | |
| | CTCTCCTGGACCCATGTGCAC | | | |
| | CCCAAGGGGCCTAAGTCATTG | | | |
| | CTGAGCCTAGAGCTGAAGGAC | | | |
| | GATCGCCCGGCCAGAGATATG | | | |
| | TGGGTAATGGAGACGGGTCTG | | | |
| | TTGTTGCCCCGGGCCACAGCT | | | |
| | CAAGACGCTGGAAAGTATTATT | | | |
| | GTCACCGTGGCAACCTGACCA | | | |
| | TGTCATTCCACCTGGAGATCAC | | | |
| | TGCTCGGCCAGTACTATGGCA | | | |
| | CTGGCTGCTGAGGACTGGTGG | | | |
| | CTGGAAGGTCTCAGCTGTGAC | | | |
| | TTTGGCTTATCTGATCTTCTGC | | | |
| | CTGTGTTCCCTTGTGGGCATTC | | | |
| | TTCATCTTCAAAGAGCCCTGGT | | | |
| | CCTGAGGAGGAAAAGAAAGCG | | | |
| | AATGACTGACCCCACCAGGAG | | | |
| | ATTC | | | |
| GSG- | GGGAGTGGG | 627 | GSG | 628 |
| linker | | | | |
| P2A | GCTACGAATTTTAGCTTGCTGA | 629 | ATNFSLLKQAGDVEENPGP | 630 |
| | AGCAGGCCGGTGATGTGGAAG | | | |
| | AGAACCCCGGGCCT | | | |
| FRBI | TGGCATGAAGGTCTGGAAGAA | 631 | WHEGLEEASRLYFGERNVKGMFE | 632 |
| | GCTTCTCGCCTTTATTTTGGCG | | VLEPLHAMMERGPQTLKETSFNQ | |
| | AACGGAACGTAAAAGGTATGTT | | AYGRDLMEAQEWCRKYMKSGNV | |
| | TGAAGTCCTGGAGCCATTGCA | | KDLLQAWDLYYHVFRRISK | |
| | CGCCATGATGGAGCGCGGGC | | | |
| | CTCAGACCCTCAAGGAAACCA | | | |
| | GTTTTAATCAGGCCTATGGGC | | | |
| | GAGACCTCATGGAGGCACAGG | | | |
| | AATGGTGTCGGAAGTATATGAA | | | |
| | GTCCGGCAACGTTAAGGATCT | | | |
| | CTTGCAGGCCTGGGACTTGTA | | | |
| | TTATCACGTGTTCCGGCGAATC | | | |
| | AGCAAG | | | |
| Linker | Cgtacg | 633 | RT | 634 |
| FRBI″ | TGGCACGAAGGTTTGGAAGAG | 635 | WHEGLEEASRLYFGERNVKGMFE | 636 |
| | GCCTCCCGCCTGTATTTCGGT | | VLEPLHAMMERGPQTLKETSFNQ | |
| | GAGAGAAATGTCAAAGGTATG | | AYGRDLMEAQEWCRKYMKSGNV | |
| | TTTGAAGTGCTTGAGCCCCTG | | KDLLQAWDLYYHVFRRISK* | |
| | CACGCCATGATGGAACGGGGG | | | |
| | CCGCAGACTCTGAAAGAAACC | | | |
| | TCATTCAACCAGGCATACGGG | | | |
| | CGAGACCTGATGGAAGCGCAG | | | |
| | GAATGGTGTAGGAAGTACATG | | | |
| | AAGTCCGGAAATGTGAAGGAC | | | |
| | TTGCTCCAGGCTTGGGACCTG | | | |
| | TACTATCACGTATTTCGGAGAA | | | |
| | TAAGCAAG-TAA | | | |

pBP0757-pSFG-iC9.T2A-dCD19.P2A-FRBₗ3

| | | SEQ | | SEQ |
|---|---|---|---|---|
| | | ID | | ID |
| Fragment | Nucleotide | NO: | Peptide | NO: |
| FKBP12v36 | ATGCTCGAGGGAGTGCAGGTG | 637 | MLEGVQVETISPGDGRTFPKRGQ | 638 |
| | GAGACTATCTCCCCAGGAGAC | | TCVVHYTGMLEDGKKVDSSRDRN | |
| | GGGCGCACCTTCCCCAAGCGC | | KPFKFMLGKQEVIRGWEEGVAQM | |
| | GGCCAGACCTGCGTGGTGCAC | | SVGQRAKLTISPDYAYGATGHPGII | |
| | TACACCGGGATGCTTGAAGAT | | PPHATLVFDVELLKLE | |
| | GGAAAGAAAGTTGATTCCTCC | | | |
| | CGGGACAGAAACAAGCCCTTT | | | |
| | AAGTTTATGCTAGGCAAGCAG | | | |
| | GAGGTGATCCGAGGCTGGGAA | | | |
| | GAAGGGGTTGCCCAGATGAGT | | | |
| | GTGGGTCAGAGAGCCAAACTG | | | |
| | ACTATATCTCCAGATTATGCCT | | | |
| | ATGGTGCCACTGGGCACCCAG | | | |
| | GCATCATCCCACCACATGCCA | | | |
| | CTCTCGTCTTCGATGTGGAGCT | | | |
| | TCTAAAACTGGAA | | | |
| Linker | TCTGGCGGTGGATCCGGA | 639 | SGGGSG | 640 |
| ΔCaspase9 | GTCGACGGATTTGGTGATGTC | 641 | VDGFGDVGALESLRGNADLAYILS | 642 |
| | GGTGCTCTTGAGAGTTTGAGG | | MEPCGHCLIINNVNFCRESGLRTR | |
| | GGAAATGCAGATTTGGCTTACA | | TGSNIDCEKLRRRFSSLHFMVEVK | |
| | TCCTGAGCATGGAGCCCTGTG | | GDLTAKKMVLALLELARQDHGALD | |
| | GCCACTGCCTCATTATCAACAA | | CCVVVILSHGCQASHLQFPGAVYG | |
| | TGTGAACTTCTGCCGTGAGTC | | TDGCPVSVEKIVNIFNGTSCPSLG | |
| | CGGGCTCCGCACCCGCACTGG | | GKPKLFFIQACGGEQKDHGFEVAS | |
| | CTCCAACATCGACTGTGAGAA | | TSPEDESPGSNPEPDATPFQEGL | |
| | GTTGCGGCGTCGCTTCTCCTC | | RTFDQLDAISSLPTPSDIFVSYSTF | |
| | GCTGCATTTCATGGTGGAGGT | | PGFVSWRDPKSGSWYVETLDDIF | |
| | GAAGGGCGACCTGACTGCCAA | | EQWAHSEDLQSLLLRVANAVSVK | |
| | GAAAATGGTGCTGGCTTTGCT | | GIYKQMPGCFNFLRKKLFFKTSAS | |
| | GGAGCTGGCGCGGCAGGACC | | RA | |
| | ACGGTGCTCTGGACTGCTGCG | | | |
| | TGGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCC | | | |
| T2A | GAGGGCAGGGGAAGTCTTCTA | 643 | EGRGSLLTCGDVEENPGP | 644 |
| | ACATGCGGGGACGTGGAGGAA | | | |
| | AATCCCGGGCCC | | | |
| ΔCD19 | ATGCCACCTCCTCGCCTCCTCT | 645 | MPPPRLLFFLLFLTPMEVRPEEPL | 646 |
| | TCTTCCTCCTCTTCCTCACCCC | | VVKVEEGDNAVLQCLKGTSDGPT | |
| | CATGGAAGTCAGGCCCGAGGA | | QQLTWSRESPLKPFLKLSLGLPGL | |
| | ACCTCTAGTGGTGAAGGTGGA | | GIHMRPLAIWLFIFNVSQQMGGFY | |
| | AGAGGGAGATAACGCTGTGCT | | LCQPGPPSEKAWQPGWTVNVEG | |
| | GCAGTGCCTCAAGGGGACCTC | | SGELFRWNVSDLGGLGCGLKNRS | |
| | AGATGGCCCCACTCAGCAGCT | | SEGPSSPSGKLMSPKLYVWAKDR | |
| | GACCTGGTCTCGGGAGTCCCC | | PEIWEGEPPCLPPRDSLNQSLSQD | |
| | GCTTAAACCCTTCTTAAAACTC | | LTMAPGSTLWLSCGVPPDSVSRG | |
| | AGCCTGGGGCTGCCAGGCCTG | | PLSWTHVHPKGPKSLLSLELKDDR | |
| | GGAATCCACATGAGGCCCCTG | | PARDMWVMETGLLLPRATAQDAG | |
| | GCCATCTGGCTTTTCATCTTCA | | KYYCHRGNLTMSFHLEITARPVLW | |
| | ACGTCTCTCAACAGATGGGGG | | HWLLRTGGWKVSAVTLAYLIFCLC | |
| | GCTTCTACCTGTGCCAGCCGG | | SLVGILHLQRALVLRRKRKRMTDP | |
| | GGCCCCCCTCTGAGAAGGCCT | | TRRF | |
| | GGCAGCCTGGCTGGACAGTCA | | | |
| | ATGTGGAGGGCAGCGGGGAG | | | |
| | CTGTTCCGGTGGAATGTTTCG | | | |
| | GACCTAGGTGGCCTGGGCTGT | | | |
| | GGCCTGAAGAACAGGTCCTCA | | | |
| | GAGGGCCCCAGCTCCCCTTCC | | | |
| | GGGAAGCTCATGAGCCCCAAG | | | |
| | CTGTATGTGTGGGCCAAAGAC | | | |
| | CGCCCTGAGATCTGGGAGGGA | | | |
| | GAGCCTCCGTGTCTCCCACCG | | | |
| | AGGGACAGCCTGAACCAGAGC | | | |
| | CTCAGCCAGGACCTCACCATG | | | |
| | GCCCCTGGCTCCACACTCTGG | | | |
| | CTGTCCTGTGGGGTACCCCCT | | | |
| | GACTCTGTGTCCAGGGGCCCC | | | |
| | CTCTCCTGGACCCATGTGCAC | | | |
| | CCCAAGGGGCCTAAGTCATTG | | | |
| | CTGAGCCTAGAGCTGAAGGAC | | | |
| | GATCGCCCGGCCAGAGATATG | | | |
| | TGGGTAATGGAGACGGGTCTG | | | |
| | TTGTTGCCCCGGGCCACAGCT | | | |
| | CAAGACGCTGGAAAGTATTATT | | | |
| | GTCACCGTGGCAACCTGACCA | | | |
| | TGTCATTCCACCTGGAGATCAC | | | |
| | TGCTCGGCCAGTACTATGGCA | | | |
| | CTGGCTGCTGAGGACTGGTGG | | | |
| | CTGGAAGGTCTCAGCTGTGAC | | | |
| | TTTGGCTTATCTGATCTTCTGC | | | |
| | CTGTGTTCCCTTGTGGGCATTC | | | |
| | TTCATCTTCAAAGAGCCCTGGT | | | |
| | CCTGAGGAGGAAAAGAAAGCG | | | |
| | AATGACTGACCCCACCAGGAG | | | |
| | ATTC | | | |
| GSG(linker) | GGGAGTGGG | 647 | GSG | 648 |
| P2A | GCTACGAATTTTAGCTTGCTGA | 649 | ATNFSLLKQAGDVEENPGP | 650 |
| | AGCAGGCCGGTGATGTGGAAG | | | |
| | AGAACCCCGGGCCT | | | |
| FRBI | TGGCATGAAGGTCTGGAAGAA | 651 | WHEGLEEASRLYFGERNVKGMFE | 652 |
| | GCTTCTCGCCTTTATTTTGGCG | | VLEPLHAMMERGPQTLKETSFNQ | |
| | AACGGAACGTAAAAGGTATGTT | | AYGRDLMEAQEWCRKYMKSGNV | |
| | TGAAGTCCTGGAGCCATTGCA | | KDLLQAWDLYYHVFRRISK | |
| | CGCCATGATGGAGCGCGGGC | | | |
| | CTCAGACCCTCAAGGAAACCA | | | |
| | GTTTTAATCAGGCCTATGGGC | | | |
| | GAGACCTCATGGAGGCACAGG | | | |
| | AATGGTGTCGGAAGTATATGAA | | | |
| | GTCCGGCAACGTTAAGGATCT | | | |
| | CTTGCAGGCCTGGGACTTGTA | | | |
| | TTATCACGTGTTCCGGCGAATC | | | |
| | AGCAAG | | | |
| Linker | Cgtacg | 653 | RT | 654 |
| FRBI′ | TGGCAcGAAGGTCTgGAcGAGG | 655 | WHEGLDEASRLYFGERNVKGMFE | 656 |
| | CTAGTAGACTGTATTTCGGCGA | | VLEPLHAMMERGPQTLKETSFNQ | |
| | GAGAAATGTAAAGGGAATGTT | | AYGRDLMEAQEWCRKYMKSGNV | |
| | CGAGGTACTGGAGCCTCTGCA | | KDLLQAWDLYYHVFRRISK | |
| | CGCCATGATGGAACGCGGCCC | | | |
| | TCAGACACTCAAGGAGACTAG | | | |
| | TTTTAACCAGGCCTATGGCAG | | | |
| | GGATCTGATGGAGGCTCAGGA | | | |
| | ATGGTGCCGGAAGTAtATGAAA | | | |
| | AGCGGTAACGTGAAGGACCTG | | | |
| | CTGCAGGCCTGGGATCTGTAT | | | |
| | TATCACGTGTTTAGAAGAATCT | | | |
| | CTAAA | | | |
| Linker | Cgtacg | 657 | RT | 658 |
| FRBI″ | TGGCACGAAGGTTTGGAAGAG | 659 | WHEGLEEASRLYFGERNVKGMFE | 660 |
| | GCCTCCCGCCTGTATTTCGGT | | VLEPLHAMMERGPQTLKETSFNQ | |
| | GAGAGAAATGTCAAAGGTATG | | AYGRDLMEAQEWCRKYMKSGNV | |
| | TTTGAAGTGCTTGAGCCCCTG | | KDLLQAWDLYYHVFRRISK* | |
| | CACGCCATGATGGAACGGGGG | | | |
| | CCGCAGACTCTGAAAGAAACC | | | |
| | TCATTCAACCAGGCATACGGG | | | |
| | CGAGACCTGATGGAAGCGCAG | | | |
| | GAATGGTGTAGGAAGTACATG | | | |
| | AAGTCCGGAAATGTGAAGGAC | | | |
| | TTGCTCCAGGCTTGGGACCTG | | | |
| | TACTATCACGTATTTCGGAGAA | | | |
| | TAAGCAAG-TAA | | | |

pBP0655-pSFG-ΔMyr.FRBₗ.MC.2A-ΔCD19

| | | SEQ ID | | SEQ |
|---|---|---|---|---|
| Fragment | Nucleotide | NO: | Peptide | ID NO: |
| FRB_{I}′ | TGGCACGAGGGGCTGGAGGA | 661 | WHEGLEEASRLYFGERNVKGMFE | 662 |
| | GGCAAGTCGACTGTATTTTGGA | | VLEPLHAMMERGPQTLKETSFNQ | |
| | GAACGCAACGTAAAGGGAATG | | AYGRDLMEAQEWCRKYMKSGNV | |
| | TTTGAGGTGCTCGAACCACTC | | KDLLQAWDLYYHVFRRISK | |
| | CATGCTATGATGGAAAGGGGG | | | |
| | CCTCAGACTCTTAAGGAAACAA | | | |
| | GTTTTAATCAAGCCTACGGACG | | | |
| | AGACCTCATGGAGGCGCAGGA | | | |
| | GTGGTGCAGAAAATACATGAA | | | |
| | ATCAGGTAATGTTAAGGACCTG | | | |
| | CTGCAGGCATGGGACCTGTAC | | | |
| | TACCATGTCTTCAGGCGCATCT | | | |
| | CAAAG | | | |
| Linker | ATGCATTCTGGTGGAGGATCA | 663 | MHSGGGSGVE | 664 |
| | GGCGTTGAA | | | |
| MyD88L | GCAGCTGGAGGCCCTGGCGC | 665 | AAGGPGAGSAAPVSSTSSLPLAAL | 666 |
| | AGGCTCTGCAGCCCCTGTATC | | NMRVRRRLSLFLNVRTQVAADWT | |
| | TAGCACCTCTTCTCTTCCTCTG | | ALAEEMDFEYLEIRQLETQADPTG | |
| | GCTGCGCTGAACATGAGAGTG | | RLLDAWQGRPGASVGRLLDLLTKL | |
| | CGGAGACGGTTGTCTTTGTTCT | | GRDDVLLELGPSIEEDCQKYILKQ | |
| | TGAATGTCAGAACACAGGTTG | | QQEEAEKPLQVAAVDSSVPRTAEL | |
| | CAGCGGACTGGACCGCTCTGG | | AGITTLDDPLGHMPERFDAFICYC | |
| | CCGAGGAAATGGACTTCGAGT | | PSDI | |
| | ACCTGGAGATCAGGCAACTCG | | | |
| | AAACGCAGGCAGATCCTACAG | | | |
| | GCAGACTGTTGGATGCGTGGC | | | |
| | AGGGACGGCCCGGAGCCAGC | | | |
| | GTTGGACGGCTCCTTGATCTTC | | | |
| | TCACCAAGCTGGGCAGAGATG | | | |
| | ACGTGCTGCTGGAATTGGGCC | | | |
| | CCAGTATTGAGGAGGACTGCC | | | |
| | AAAAATACATCTTGAAGCAGCA | | | |
| | ACAGGAGGAGGCGGAGAAGC | | | |
| | CCCTCCAGGTCGCAGCCGTCG | | | |
| | ATTCATCCGTGCCTAGAACAG | | | |
| | CCGAACTTGCAGGCATCACTA | | | |
| | CCCTGGATGATCCCCTGGGCC | | | |
| | ATATGCCAGAGAGGTTTGATG | | | |
| | CGTTTATCTGCTATTGCCCAAG | | | |
| | CGATATC | | | |
| Linker | GTTGAG | 667 | VE | 668 |
| hCD40 | AAGAAGGTGGCCAAGAAGCCA | 669 | KKVAKKPTNKAPHPKQEPQEINFP | 670 |
| | ACCAATAAAGCTCCACATCCTA | | DDLPGSNTAAPVQETLHGCQPVT | |
| | AACAGGAGCCACAAGAAATCA | | QEDGKESRISVQERQ | |
| | ACTTTCCAGATGATCTCCCTGG | | | |
| | CTCTAATACTGCAGCCCCCGT | | | |
| | GCAGGAAACCCTGCACGGCTG | | | |
| | TCAACCTGTGACACAGGAAGA | | | |
| | CGGGAAGGAAAGCAGGATATC | | | |
| | CGTGCAGGAACGGCAA | | | |
| Linker | GTCGAC | 671 | VD | 672 |
| HA | TACCCATACGACGTGCCAGAT | 673 | YPYDVPDYA | 674 |
| epitope | TATGCT | | | |
| Linker | CCGCGG | 675 | PR | 676 |
| T2A | GAAGGCCGAGGGAGCCTGCT | 677 | EGRGSLLTCGDVEENPGP | 678 |
| | GACATGTGGCGATGTGGAGGA | | | |
| | AAACCCAGGACCA | | | |
| ΔCD19 | ATGCCACCACCTCGCCTGCTG | 679 | MPPPRLLFFLLFLTPMEVRPEEPL | 680 |
| | TTCTTTCTGCTGTTCCTGACAC | | VVKVEEGDNAVLQCLKGTSDGPT | |
| | CTATGGAGGTGCGACCTGAGG | | QQLTWSRESPLKPFLKLSLGLPGL | |
| | AACCACTGGTCGTGAAGGTCG | | GIHMRPLAIWLFIFNVSQQMGGFY | |
| | AGGAAGGCGACAATGCCGTGC | | LCQPGPPSEKAWQPGWTVNVEG | |
| | TGCAGTGCCTGAAAGGCACTT | | SGELFRWNVSDLGGLGCGLKNRS | |
| | CTGATGGGCCAACTCAGCAGC | | SEGPSSPSGKLMSPKLYVWAKDR | |
| | TGACCTGGTCCAGGGAGTCTC | | PEIWEGEPPCLPPRDSLNQSLSQD | |
| | CCCTGAAGCCTTTTCTGAAACT | | LTMAPGSTLWLSCGVPPDSVSRG | |
| | GAGCCTGGGACTGCCAGGACT | | PLSWTHVHPKGPKSLLSLELKDDR | |
| | GGGAATCCACATGCGCCCTCT | | PARDMWVMETGLLLPRATAQDAG | |
| | GGCTATCTGGCTGTTCATCTTC | | KYYCHRGNLTMSFHLEITARPVLW | |
| | AACGTGAGCCAGCAGATGGGA | | HWLLRTGGWKVSAVTLAYLIFCLC | |
| | GGATTCTACCTGTGCCAGCCA | | SLVGILHLQRALVLRRKRKRMTDP | |
| | GGACCACCATCCGAGAAGGCC | | TRRF* | |
| | TGGCAGCCTGGATGGACCGTC | | | |
| | AACGTGGAGGGGTCTGGAGAA | | | |
| | CTGTTTAGGTGGAATGTGAGT | | | |
| | GACCTGGGAGGACTGGGATGT | | | |
| | GGGCTGAAGAACCGCTCCTCT | | | |
| | GAAGGCCCAAGTTCACCCTCA | | | |
| | GGGAAGCTGATGAGCCCAAAA | | | |
| | CTGTACGTGTGGGCCAAAGAT | | | |
| | CGGCCCGAGATCTGGGAGGG | | | |
| | AGAACCTCCATGCCTGCCACC | | | |
| | TAGAGACAGCCTGAATCAGAG | | | |
| | TCTGTCACAGGATCTGACAATG | | | |
| | GCCCCCGGGTCCACTCTGTGG | | | |
| | CTGTCTTGTGGAGTCCCACCC | | | |
| | GACAGCGTGTCCAGAGGCCCT | | | |
| | CTGTCCTGGACCCACGTGCAT | | | |
| | CCTAAGGGGCCAAAAAGTCTG | | | |
| | CTGTCACTGGAACTGAAGGAC | | | |
| | GATCGGCCTGCCAGAGACATG | | | |
| | TGGGTCATGGAGACTGGACTG | | | |
| | CTGCTGCCACGAGCAACCGCA | | | |
| | CAGGATGCTGGAAAATACTATT | | | |
| | GCCACCGGGGCAATCTGACAA | | | |
| | TGTCCTTCCATCTGGAGATCAC | | | |
| | TGCAAGGCCCGTGCTGTGGCA | | | |
| | CTGGCTGCTGCGAACCGGAGG | | | |
| | ATGGAAGGTCAGTGCTGTGAC | | | |
| | ACTGGCATATCTGATCTTTTGC | | | |
| | CTGTGCTCCCTGGTGGGCATT | | | |
| | CTGCATCTGCAGAGAGCCCTG | | | |
| | GTGCTGCGGAGAAAGAGAAAG | | | |
| | AGAATGACTGACCCAACAAGA | | | |
| | AGGTTTTGA | | | |

pBP0498-pSFG-ΔMyriMC.FRBₗ2. P2A-ΔCD19

| | | SEQ | | SEQ |
|---|---|---|---|---|
| | | ID | | ID |
| Fragment | Nucleotide | NO: | Peptide | NO: |
| Start | ATGCTCGAG | 681 | MLE | 682 |
| FRB_{I}\hat{ } | TGGCACGAGGGGCTGGAGGA | 683 | WHEGLEEASRLYFGERNVKGMFE | 684 |
| | GGCAAGTCGACTGTATTTTGGA | | VLEPLHAMMERGPQTLKETSFNQ | |
| | GAACGCAACGTAAAGGGAATG | | AYGRDLMEAQEWCRKYMKSGNV | |
| | TTTGAGGTGCTCGAACCACTC | | KDLLQAWDLYYHVFRRISK | |
| | CATGCTATGATGGAAAGGGGG | | | |
| | CCTCAGACTCTTAAGGAAACAA | | | |
| | GTTTTAATCAAGCCTACGGACG | | | |
| | AGACCTCATGGAGGCGCAGGA | | | |
| | GTGGTGCAGAAAATACATGAA | | | |
| | ATCAGGTAATGTTAAGGACCTG | | | |
| | CTGCAGGCATGGGACCTGTAC | | | |
| | TACCATGTCTTCAGGCGCATCT | | | |
| | CAAAG | | | |
| Linker | ATGCAT | 685 | MH | 686 |
| FRB_{I}\hat{ }\hat{ } | TGGCACGAAGGCCTGGAAGAG | 687 | WHEGLEEASRLYFGERNVKGMFE | 688 |
| | GCCTCAAGACTTTACTTTGGTG | | VLEPLHAMMERGPQTLKETSFNQ | |
| | AACGCAACGTTAAAGGCATGTT | | AYGRDLMEAQEWCRKYMKSGNV | |
| | CGAGGTGCTGGAACCCTTGCA | | KDLLQAWDLYYHVFRRISK | |
| | TGCAATGATGGAGCGAGGTCC | | | |
| | TCAGACACTCAAAGAGACATCT | | | |
| | TTTAACCAGGCGTATGGACGG | | | |
| | GACCTCATGGAGGCTCAGGAA | | | |
| | TGGTGCCGCAAGTACATGAAA | | | |
| | AGTGGGAATGTGAAGGATCTG | | | |
| | CTGCAAGCATGGGATCTGTATT | | | |
| | ACCACGTGTTTAGACGGATCA | | | |
| | GCAAA | | | |
| Linker | ATGCATTCTGGTGGAGGATCA | 689 | MHSGGGSGVE | 690 |
| | GGCGTTGAA | | | |
| MyD88L | GCAGCTGGAGGCCCTGGCGC | 691 | AAGGPGAGSAAPVSSTSSLPLAAL | 692 |
| | AGGCTCTGCAGCCCCTGTATC | | NMRVRRRLSLFLNVRTQVAADWT | |
| | TAGCACCTCTTCTCTTCCTCTG | | ALAEEMDFEYLEIRQLETQADPTG | |
| | GCTGCGCTGAACATGAGAGTG | | RLLDAWQGRPGASVGRLLDLLTKL | |
| | CGGAGACGGTTGTCTTTGTTCT | | GRDDVLLELGPSIEEDCQKYILKQ | |
| | TGAATGTCAGAACACAGGTTG | | QQEEAEKPLQVAAVDSSVPRTAEL | |
| | CAGCGGACTGGACCGCTCTGG | | AGITTLDDPLGHMPERFDAFICYC | |
| | CCGAGGAAATGGACTTCGAGT | | PSDI | |
| | ACCTGGAGATCAGGCAACTCG | | | |
| | AAACGCAGGCAGATCCTACAG | | | |
| | GCAGACTGTTGGATGCGTGGC | | | |
| | AGGGACGGCCCGGAGCCAGC | | | |
| | GTTGGACGGCTCCTTGATCTTC | | | |
| | TCACCAAGCTGGGCAGAGATG | | | |
| | ACGTGCTGCTGGAATTGGGCC | | | |
| | CCAGTATTGAGGAGGACTGCC | | | |
| | AAAAATACATCTTGAAGCAGCA | | | |
| | ACAGGAGGAGGCGGAGAAGC | | | |
| | CCCTCCAGGTCGCAGCCGTCG | | | |
| | ATTCATCCGTGCCTAGAACAG | | | |
| | CCGAACTTGCAGGCATCACTA | | | |
| | CCCTGGATGATCCCCTGGGCC | | | |
| | ATATGCCAGAGAGGTTTGATG | | | |
| | CGTTTATCTGCTATTGCCCAAG | | | |
| | CGATATC | | | |
| Linker | GTTGAG | 693 | VE | 694 |
| hCD40 | AAGAAGGTGGCCAAGAAGCCA | 695 | KKVAKKPTNKAPHPKQEPQEINFP | 696 |
| | ACCAATAAAGCTCCACATCCTA | | DDLPGSNTAAPVQETLHGCQPVT | |
| | AACAGGAGCCACAAGAAATCA | | QEDGKESRISVQERQ | |
| | ACTTTCCAGATGATCTCCCTGG | | | |
| | CTCTAATACTGCAGCCCCCGT | | | |
| | GCAGGAAACCCTGCACGGCTG | | | |
| | TCAACCTGTGACACAGGAAGA | | | |
| | CGGGAAGGAAAGCAGGATATC | | | |
| | CGTGCAGGAACGGCAA | | | |
| Linker | GTCGAC | 697 | VD | 698 |
| HA | TACCCATACGACGTGCCAGAT | 699 | YPYDVPDYA | 700 |
| | TATGCT | | | |
| Linker | CCGCGG | 701 | PR | 702 |
| T2A | GAAGGCCGAGGGAGCCTGCT | 703 | EGRGSLLTCGDVEENPGP | 704 |
| | GACATGTGGCGATGTGGAGGA | | | |
| | AAACCCAGGACCA | | | |
| ΔCD19 | ATGCCACCACCTCGCCTGCTG | 705 | MPPPRLLFFLLFLTPMEVRPEEPL | 706 |
| | TTCTTTCTGCTGTTCCTGACAC | | VVKVEEGDNAVLQCLKGTSDGPT | |
| | CTATGGAGGTGCGACCTGAGG | | QQLTWSRESPLKPFLKLSLGLPGL | |
| | AACCACTGGTCGTGAAGGTCG | | GIHMRPLAIWLFIFNVSQQMGGFY | |
| | AGGAAGGCGACAATGCCGTGC | | LCQPGPPSEKAWQPGWTVNVEG | |
| | TGCAGTGCCTGAAAGGCACTT | | SGELFRWNVSDLGGLGCGLKNRS | |
| | CTGATGGGCCAACTCAGCAGC | | SEGPSSPSGKLMSPKLYVWAKDR | |
| | TGACCTGGTCCAGGGAGTCTC | | PEIWEGEPPCLPPRDSLNQSLSQD | |
| | CCCTGAAGCCTTTTCTGAAACT | | LTMAPGSTLWLSCGVPPDSVSRG | |
| | GAGCCTGGGACTGCCAGGACT | | PLSWTHVHPKGPKSLLSLELKDDR | |
| | GGGAATCCACATGCGCCCTCT | | PARDMWVMETGLLLPRATAQDAG | |
| | GGCTATCTGGCTGTTCATCTTC | | KYYCHRGNLTMSFHLEITARPVLW | |
| | AACGTGAGCCAGCAGATGGGA | | HWLLRTGGWKVSAVTLAYLIFCLC | |
| | GGATTCTACCTGTGCCAGCCA | | SLVGILHLQRALVLRRKRKRMTDP | |
| | GGACCACCATCCGAGAAGGCC | | TRRF* | |
| | TGGCAGCCTGGATGGACCGTC | | | |
| | AACGTGGAGGGGTCTGGAGAA | | | |
| | CTGTTTAGGTGGAATGTGAGT | | | |
| | GACCTGGGAGGACTGGGATGT | | | |
| | GGGCTGAAGAACCGCTCCTCT | | | |
| | GAAGGCCCAAGTTCACCCTCA | | | |
| | GGGAAGCTGATGAGCCCAAAA | | | |
| | CTGTACGTGTGGGCCAAAGAT | | | |
| | CGGCCCGAGATCTGGGAGGG | | | |
| | AGAACCTCCATGCCTGCCACC | | | |
| | TAGAGACAGCCTGAATCAGAG | | | |
| | TCTGTCACAGGATCTGACAATG | | | |
| | GCCCCCGGGTCCACTCTGTGG | | | |
| | CTGTCTTGTGGAGTCCCACCC | | | |
| | GACAGCGTGTCCAGAGGCCCT | | | |
| | CTGTCCTGGACCCACGTGCAT | | | |
| | CCTAAGGGGCCAAAAAGTCTG | | | |
| | CTGTCACTGGAACTGAAGGAC | | | |
| | GATCGGCCTGCCAGAGACATG | | | |
| | TGGGTCATGGAGACTGGACTG | | | |
| | CTGCTGCCACGAGCAACCGCA | | | |
| | CAGGATGCTGGAAAATACTATT | | | |
| | GCCACCGGGGCAATCTGACAA | | | |
| | TGTCCTTCCATCTGGAGATCAC | | | |
| | TGCAAGGCCCGTGCTGTGGCA | | | |
| | CTGGCTGCTGCGAACCGGAGG | | | |
| | ATGGAAGGTCAGTGCTGTGAC | | | |
| | ACTGGCATATCTGATCTTTTGC | | | |
| | CTGTGCTCCCTGGTGGGCATT | | | |
| | CTGCATCTGCAGAGAGCCCTG | | | |
| | GTGCTGCGGAGAAAGAGAAAG | | | |
| | AGAATGACTGACCCAACAAGA | | | |
| | AGGTTTTGA | | | |

pBP0488-pSFG-αHER2.Q.8stm.CD3zeta.Fpk2

| | | SEQ | | SEQ |
|---|---|---|---|---|
| | | ID | | ID |
| Fragment | Nucleotide | NO: | Peptide | NO: |
| Signal | ATGGAGTTTGGACTTTCTTGGT | 707 | MEFGLSVVLFLVAILKGVQCSR | 708 |
| Peptide | TGTTTTTGGTGGCAATTCTGAA | | | |
| | GGGTGTCCAGTGTAGCAGG | | | |
| FRP5-VL | GACATCCAATTGACACAATCAC | 709 | DIQLTQSHKFLSTSVGDRVSITCKA | 710 |
| | ACAAATTTCTCTCAACTTCTGT | | SQDVYNAVAWYQQKPGQSPKLLI | |
| | AGGAGACAGAGTGAGCATAAC | | YSASSRYTGVPSRFTGSGSGPDF | |
| | CTGCAAAGCATCCCAGGACGT | | TFTISSVQAEDLAVYFCQQHFRTP | |
| | GTACAATGCTGTGGCTTGGTA | | FTFGSGTKLEIKAL | |
| | CCAACAGAAGCCTGGACAATC | | | |
| | CCCAAAATTGCTGATTTATTCT | | | |
| | GCCTCTAGTAGGTACACTGGG | | | |
| | GTACCTTCTCGGTTTACGGGCT | | | |
| | CTGGGTCCGGACCAGATTTCA | | | |
| | CGTTCACAATCAGTTCCGTTCA | | | |
| | AGCTGAAGACCTCGCTGTTTAT | | | |
| | TTTTGCCAGCAGCACTTCCGAA | | | |
| | CCCCTTTTACTTTTGGCTCAGG | | | |
| | CACTAAGTTGGAAATCAAGGCT | | | |
| | TTG | | | |
| Linker | GGCGGAGGAAGCGGAGGTGG | 711 | GGGSGGGG | 712 |
| | GGGC | | | |
| FRP5-VH | GAAGTCCAATTGCAACAGTCA | 713 | EVQLQQSGPELKKPGETVKISCKA | 714 |
| | GGCCCCGAATTGAAAAAGCCC | | SGYPFTNYGMNVVVKQAPGQGLK | |
| | GGCGAAACAGTGAAGATATCT | | VVMGWINTSTGESTFADDFKGRFD | |
| | TGTAAAGCCTCCGGTTACCCTT | | FSLETSANTAYLQINNLKSEDMAT | |
| | TTACGAACTATGGAATGAACTG | | YFCARVVEVYHGYVPYWGQGTTV | |
| | GGTCAAACAAGCCCCTGGACA | | TVSS | |
| | GGGATTGAAGTGGATGGGATG | | | |
| | GATCAATACATCAACAGGCGA | | | |
| | GTCTACCTTCGCAGATGATTTC | | | |
| | AAAGGTCGCTTTGACTTCTCAC | | | |
| | TGGAGACCAGTGCAAATACCG | | | |
| | CCTACCTTCAGATTAACAATCT | | | |
| | TAAAAGCGAGGATATGGCAAC | | | |
| | CTACTTTTGCGCAAGATGGGA | | | |
| | AGTTTATCACGGGTACGTGCC | | | |
| | ATACTGGGGACAAGGAACGAC | | | |
| | AGTGACAGTTAGTAGC | | | |
| Linker | GGATCC | 715 | GS | 716 |
| Q-Bend-10 | GAACTTCCTACTCAGGGGACTT | 717 | ELPTQGTFSNVSTNVS | 718 |
| (CD34 | TCTCAAACGTTAGCACAAACGT | | | |
| Epitope) | AAGT | | | |
| CD8 Stalk | CCCGCCCCAAGACCCCCCACA | 719 | PAPRPPTPAPTIASQPLSLRPEAC | 720 |
| | CCTGCGCCGACCATTGCTTCT | | RPAAGGAVHTRGLDFACD | |
| | CAACCCCTGAGTTTGAGACCC | | | |
| | GAGGCCTGCCGGCCAGCTGC | | | |
| | CGGCGGGGCCGTGCATACAA | | | |
| | GAGGACTCGATTTCGCTTGCG | | | |
| | AC | | | |
| CD8a tm | ATCTATATCTGGGCACCTCTCG | 721 | IYIWAPLAGTCGVLLLSLVITLYCNH | 722 |
| | CTGGCACCTGTGGAGTCCTTC | | RNRRRVCKCPR | |
| | TGCTCAGCCTGGTTATTACTCT | | | |
| | GTACTGTAATCACCGGAATCG | | | |
| | CCGCCGCGTTTGTAAGTGTCC | | | |
| | CAGG | | | |
| Linker | CTCGAG | 723 | LE | 724 |
| CD3 zeta | AGAGTGAAGTTCAGCAGGAGC | 725 | RVKFSRSADAPAYQQGQNQLYNE | 726 |
| | GCAGACGCCCCCGCGTACCAG | | LNLGRREEYDVLDKRRGRDPEMG | |
| | CAGGGCCAGAACCAGCTCTAT | | GKPRRKNPQEGLYNELQKDKMAE | |
| | AACGAGCTCAATCTAGGACGA | | AYSEIGMKGERRRGKGHDGLYQG | |
| | AGAGAGGAGTACGATGTTTTG | | LSTATKDTYDALHMQALPP | |
| | GACAAGAGACGTGGCCGGGA | | | |
| | CCCTGAGATGGGGGGAAAGCC | | | |
| | GAGAAGGAAGAACCCTCAGGA | | | |
| | AGGCCTGTACAATGAACTGCA | | | |
| | GAAAGATAAGATGGCGGAGGC | | | |
| | CTACAGTGAGATTGGGATGAA | | | |
| | AGGCGAGCGCCGGAGGGGCA | | | |
| | AGGGGCACGATGGCCTTTACC | | | |
| | AGGGTCTCAGTACAGCCACCA | | | |
| | AGGACACCTACGACGCCCTTC | | | |
| | ACATGCAAGCTCTTCCACCTCG | | | |
| Linker | TCAGGCGGTGGCTCAGGTGTT | 727 | SGGGSGVN | 728 |
| | AAC | | | |
| Fpk′ | GGCGTCCAAGTCGAAACCATT | 729 | GVQVETISPGDGRTFPKRGQTCV | 730 |
| | AGTCCCGGCGATGGCAGAACA | | VHYTGMLEDGKKFDSSRDRNKPF | |
| | TTTCCTAAAAGGGGACAAACAT | | KFMLGKQEVIRGVVEEGVAQMSVG | |
| | GTGTCGTCCATTATACAGGCAT | | QRAKLTISPDYAYGATGHPPKIPP | |
| | GTTGGAGGACGGCAAAAAGTT | | HATLVFDVELLKLE | |
| | CGACAGTAGTAGAGATCGCAA | | | |
| | TAAACCTTTCAAATTCATGTTG | | | |
| | GGAAAACAAGAAGTCATTAGG | | | |
| | GGATGGGAGGAGGGCGTGGC | | | |
| | TCAAATGTCCGTCGGCCAACG | | | |
| | CGCTAAGCTCACCATCAGCCC | | | |
| | CGACTACGCATACGGCGCTAC | | | |
| | CGGACATCCCCCTAAGATTCC | | | |
| | CCCTCACGCTACCTTGGTGTTT | | | |
| | GACGTCGAACTGTTGAAGCTC | | | |
| | GAA | | | |
| Linker | GTTAAC | 731 | VN | 732 |
| Fpk | GGAGTGCAGGTGGAGACTATC | 733 | GVQVETISPGDGRTFPKRGQTCV | 734 |
| | TCCCCAGGAGACGGGCGCAC | | VHYTGMLEDGKKFDSSRDRNKPF | |
| | CTTCCCCAAGCGCGGCCAGAC | | KFMLGKQEVIRGVVEEGVAQMSVG | |
| | CTGCGTGGTGCACTACACCGG | | QRAKLTISPDYAYGATGHPPKIPP | |
| | GATGCTTGAAGATGGAAAGAA | | HATLVFDVELLKLE | |
| | ATTCGATTCCTCTCGGGACAGA | | | |
| | AACAAGCCCTTTAAGTTTATGC | | | |
| | TAGGCAAGCAGGAGGTGATCC | | | |
| | GAGGCTGGGAAGAAGGGGTT | | | |
| | GCCCAGATGAGTGTGGGTCAG | | | |
| | AGAGCCAAACTGACTATATCTC | | | |
| | CAGATTATGCCTATGGTGCCAC | | | |
| | TGGGCACCCACCTAAGATCCC | | | |
| | ACCACATGCCACTCTCGTCTTC | | | |
| | GATGTGGAGCTTCTAAAACTG | | | |
| | GAA | | | |
| GSG | GGATCGGGA | 735 | GSG | 736 |
| Linker | | | | |
| P2A | GCTACTAACTTCAGCCTGCTGA | 737 | ATNFSLLKQAGDVEENPGP | 738 |
| | AGCAGGCTGGAGACGTGGAG | | | |
| | GAGAACCCCGGGCCT | | | |

pBP0467-pSH1-FRBI′.FRBI.LS.ΔCaspase9

| | | SEQ | | SEQ |
|---|---|---|---|---|
| | | ID | | ID |
| Fragment | Nucleotide | NO: | Peptide | NO: |
| FRB_{I}′ | TGGCATGAAGGCCTGGAAGAG | 739 | WHEGLEEASRLYFGERNVKGMFE | 740 |
| | GCATCTCGTTTGTACTTTGGGG | | VLEPLHAMMERGPQTLKETSFNQ | |
| | AAAGGAACGTGAAAGGCATGT | | AYGRDLMEAQEWCRKYMKSGNV | |
| | TTGAGGTGCTGGAGCCCTTGC | | KDLLQAWDLYYHVFRRISK | |
| | ACGCTATGATGGAACGGGGCC | | | |
| | CCCAGACTCTGAAGGAAACAT | | | |
| | CCTTTAATCAGGCCTATGGTCG | | | |
| | AGATTTAATGGAGGCCCAAGA | | | |
| | GTGGTGCAGGAAGTACATGAA | | | |
| | ATCAGGGAATGTCAAGGACCT | | | |
| | CCTCCAAGCCTGGGACCTCTA | | | |
| | TTATCATGTGTTCCGACGAATC | | | |
| | TCAAAG | | | |
| Linker | GTCGAG | 741 | VE | 742 |
| FRB_{I} | TGGCATGAAGGGTTGGAAGAA | 743 | WHEGLEEASRLYFGERNVKGMFE | 744 |
| | GCTTCAAGGCTGTACTTCGGA | | VLEPLHAMMERGPQTLKETSFNQ | |
| | GAGAGGAACGTGAAGGGCATG | | AYGRDLMEAQEWCRKYMKSGNV | |
| | TTTGAGGTTCTTGAACCTCTGC | | KDLLQAWDLYYHVFRRISK | |
| | ACGCCATGATGGAACGGGGAC | | | |
| | CGCAGACACTGAAAGAAACCT | | | |
| | CTTTTAATCAGGCCTACGGCAG | | | |
| | AGACCTGATGGAGGCCCAAGA | | | |
| | ATGGTGTAGAAAGTATATGAAA | | | |
| | TCCGGTAACGTGAAAGACCTG | | | |
| | CTCCAGGCCTGGGACCTTTATT | | | |
| | ACCATGTGTTCAGGCGGATCA | | | |
| | GTAAG | | | |
| Linker | TCAGGCGGTGGCTCAGGT | 745 | SGGGSG | 746 |
| ΔCaspase9 | GTCGACGGATTTGGTGATGTC | 747 | VDGFGDVGALESLRGNADLAYILS | 748 |
| | GGTGCTCTTGAGAGTTTGAGG | | MEPCGHCLIINNVNFCRESGLRTR | |
| | GGAAATGCAGATTTGGCTTACA | | TGSNIDCEKLRRRFSSLHFMVEVK | |
| | TCCTGAGCATGGAGCCCTGTG | | GDLTAKKMVLALLELARQDHGALD | |
| | GCCACTGCCTCATTATCAACAA | | CCVVVILSHGCQASHLQFPGAVYG | |
| | TGTGAACTTCTGCCGTGAGTC | | TDGCPVSVEKIVNIFNGTSCPSLG | |
| | CGGGCTCCGCACCCGCACTGG | | GKPKLFFIQACGGEQKDHGFEVAS | |
| | CTCCAACATCGACTGTGAGAA | | TSPEDESPGSNPEPDATPFQEGL | |
| | GTTGCGGCGTCGCTTCTCCTC | | RTFDQLDAISSLPTPSDIFVSYSTF | |
| | GCTGCATTTCATGGTGGAGGT | | PGFVSWRDPKSGSWYVETLDDIF | |
| | GAAGGGCGACCTGACTGCCAA | | EQWAHSEDLQSLLLRVANAVSVK | |
| | GAAAATGGTGCTGGCTTTGCT | | GIYKQMPGCFNFLRKKLFFKTSAS | |
| | GGAGCTGGCGCgGCAGGACCA | | RAEGRGSLLTCGDVEENPGP* | |
| | CGGTGCTCTGGACTGCTGCGT | | | |
| | GGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCCGAGGGCA | | | |
| | GGGGAAGTCTTCTAACATGCG | | | |
| | GGGACGTGGAGGAAAATCCCG | | | |
| | GGCCCTGA | | | |

pBP0606-pSFG-k-ΔMyriMC.2A-ΔCD19

| | | SEQ | | SEQ |
|---|---|---|---|---|
| | | ID | | ID |
| Fragment | Nucleotide | NO: | Peptide | NO: |
| MyD88 | ATGGCTGCAGGAGGTCCCGGC | 749 | MAAGGPGAGSAAPVSSTSSLPLA | 750 |
| | GCGGGGTCTGCGGCCCCGGT | | ALNMRVRRRLSLFLNVRTQVAAD | |
| | CTCCTCCACATCCTCCCTTCCC | | WTALAEEMDFEYLEIRQLETQAD | |
| | CTGGCTGCTCTCAACATGCGA | | PTGRLLDAWQGRPGASVGRLLD | |
| | GTGCGGCGCCGCCTGTCTCTG | | LLTKLGRDDVLLELGPSIEEDCQK | |
| | TTCTTGAACGTGCGGACACAG | | YILKQQQEEAEKPLQVAAVDSSV | |
| | GTGGCGGCCGACTGGACCGC | | PRTAELAGITTLDDPLGHMPERF | |
| | GCTGGCGGAGGAGATGGACTT | | DAFICYCPSDI | |
| | TGAGTACTTGGAGATCCGGCA | | | |
| | ACTGGAGACACAAGCGGACCC | | | |
| | CACTGGCAGGCTGCTGGACGC | | | |
| | CTGGCAGGGACGCCCTGGCG | | | |
| | CCTCTGTAGGCCGACTGCTCG | | | |
| | ATCTGCTTACCAAGCTGGGCC | | | |
| | GCGACGACGTGCTGCTGGAGC | | | |
| | TGGGACCCAGCATTGAGGAGG | | | |
| | ATTGCCAAAAGTATATCTTGAA | | | |
| | GCAGCAGCAGGAGGAGGCTG | | | |
| | AGAAGCCTTTACAGGTGGCCG | | | |
| | CTGTAGACAGCAGTGTCCCAC | | | |
| | GGACAGCAGAGCTGGCGGGC | | | |
| | ATCACCACACTTGATGACCCC | | | |
| | CTGGGGCATATGCCTGAGCGT | | | |
| | TTCGATGCCTTCATCTGCTATT | | | |
| | GCCCCAGCGACATC | | | |
| Linker | GTCGAG | 751 | VG | 752 |
| hCD40 | AAAAAGGTGGCCAAGAAGCCA | 753 | KKVAKKPTNKAPHPKQEPQEINF | 754 |
| | ACCAATAAGGCCCCCCACCCC | | PDDLPGSNTAAPVQETLHGCQP | |
| | AAGCAGGAGCCCCAGGAGATC | | VTQEDGKESRISVQERQ | |
| | AATTTTCCCGACGATCTTCCTG | | | |
| | GCTCCAACACTGCTGCTCCAG | | | |
| | TGCAGGAGACTTTACATGGAT | | | |
| | GCCAACCGGTCACCCAGGAGG | | | |
| | ATGGCAAAGAGAGTCGCATCT | | | |
| | CAGTGCAGGAGAGACAG | | | |
| Linker | GTCGAG | 755 | VG | 756 |
| Fv′ | GGCGTCCAAGTCGAAACCATT | 757 | GVQVETISPGDGRTFPKRGQTCV | 758 |
| | AGTCCCGGCGATGGCAGAACA | | VHYTGMLEDGKKVDSSRDRNKP | |
| | TTTCCTAAAAGGGGACAAACAT | | FKFMLGKQEVIRGWEEGVAQMS | |
| | GTGTCGTCCATTATACAGGCAT | | VGQRAKLTISPDYAYGATGHPGII | |
| | GTTGGAGGACGGCAAAAAGGT | | PPHATLVFDVELLKLE | |
| | GGACAGTAGTAGAGATCGCAA | | | |
| | TAAACCTTTCAAATTCATGTTG | | | |
| | GGAAAACAAGAAGTCATTAGG | | | |
| | GGATGGGAGGAGGGCGTGGC | | | |
| | TCAAATGTCCGTCGGCCAACG | | | |
| | CGCTAAGCTCACCATCAGCCC | | | |
| | CGACTACGCATACGGCGCTAC | | | |
| | CGGACATCCCGGAATTATTCC | | | |
| | CCCTCACGCTACCTTGGTGTTT | | | |
| | GACGTCGAACTGTTGAAGCTC | | | |
| | GAA | | | |
| Linker | GTCGAG | 759 | VG | 760 |
| Fv | GGAGTGCAGGTGGAGACTATC | 761 | GVQVETISPGDGRTFPKRGQTCV | 762 |
| | TCCCCAGGAGACGGGCGCAC | | VHYTGMLEDGKKVDSSRDRNKP | |
| | CTTCCCCAAGCGCGGCCAGAC | | FKFMLGKQEVIRGWEEGVAQMS | |
| | CTGCGTGGTGCACTACACCGG | | VGQRAKLTISPDYAYGATGHPGII | |
| | GATGCTTGAAGATGGAAAGAA | | PPHATLVFDVELLKLE | |
| | AGTTGATTCCTCCCGGGACAG | | | |
| | AAACAAGCCCTTTAAGTTTATG | | | |
| | CTAGGCAAGCAGGAGGTGATC | | | |
| | CGAGGCTGGGAAGAAGGGGTT | | | |
| | GCCCAGATGAGTGTGGGTCAG | | | |
| | AGAGCCAAACTGACTATATCTC | | | |
| | CAGATTATGCCTATGGTGCCAC | | | |
| | TGGGCACCCAGGCATCATCCC | | | |
| | ACCACATGCCACTCTCGTCTTC | | | |
| | GATGTGGAGCTTCTAAAACTG | | | |
| | GAA | | | |
| Linker | CCGCGG | 763 | PR | 764 |
| T2A | GAAGGCCGAGGGAGCCTGCT | 765 | EGRGSLLTCGDVEENPGP | 766 |
| | GACATGTGGCGATGTGGAGGA | | | |
| | AAACCCAGGACCA | | | |
| ΔCD19 | ATGCCACCACCTCGCCTGCTG | 767 | MPPPRLLFFLLFLTPMEVRPEEPL | 768 |
| | TTCTTTCTGCTGTTCCTGACAC | | VVKVEEGDNAVLQCLKGTSDGPT | |
| | CTATGGAGGTGCGACCTGAGG | | QQLTWSRESPLKPFLKLSLGLPG | |
| | AACCACTGGTCGTGAAGGTCG | | LGIHMRPLAIWLFIFNVSQQMGGF | |
| | AGGAAGGCGACAATGCCGTGC | | YLCQPGPPSEKAWQPGWTVNVE | |
| | TGCAGTGCCTGAAAGGCACTT | | GSGELFRWNVSDLGGLGCGLKN | |
| | CTGATGGGCCAACTCAGCAGC | | RSSEGPSSPSGKLMSPKLYVWA | |
| | TGACCTGGTCCAGGGAGTCTC | | KDRPEIWEGEPPCLPPRDSLNQS | |
| | CCCTGAAGCCTTTTCTGAAACT | | LSQDLTMAPGSTLWLSCGVPPD | |
| | GAGCCTGGGACTGCCAGGACT | | SVSRGPLSWTHVHPKGPKSLLSL | |
| | GGGAATCCACATGCGCCCTCT | | ELKDDRPARDMWVMETGLLLPR | |
| | GGCTATCTGGCTGTTCATCTTC | | ATAQDAGKYYCHRGNLTMSFHL | |
| | AACGTGAGCCAGCAGATGGGA | | EITARPVLWHWLLRTGGWKVSA | |
| | GGATTCTACCTGTGCCAGCCA | | VTLAYLIFCLCSLVGILHLQRALVL | |
| | GGACCACCATCCGAGAAGGCC | | RRKRKRMTDPTRRF* | |
| | TGGCAGCCTGGATGGACCGTC | | | |
| | AACGTGGAGGGGTCTGGAGAA | | | |
| | CTGTTTAGGTGGAATGTGAGT | | | |
| | GACCTGGGAGGACTGGGATGT | | | |
| | GGGCTGAAGAACCGCTCCTCT | | | |
| | GAAGGCCCAAGTTCACCCTCA | | | |
| | GGGAAGCTGATGAGCCCAAAA | | | |
| | CTGTACGTGTGGGCCAAAGAT | | | |
| | CGGCCCGAGATCTGGGAGGG | | | |
| | AGAACCTCCATGCCTGCCACC | | | |
| | TAGAGACAGCCTGAATCAGAG | | | |
| | TCTGTCACAGGATCTGACAATG | | | |
| | GCCCCCGGGTCCACTCTGTGG | | | |
| | CTGTCTTGTGGAGTCCCACCC | | | |
| | GACAGCGTGTCCAGAGGCCCT | | | |
| | CTGTCCTGGACCCACGTGCAT | | | |
| | CCTAAGGGGCCAAAAAGTCTG | | | |
| | CTGTCACTGGAACTGAAGGAC | | | |
| | GATCGGCCTGCCAGAGACATG | | | |
| | TGGGTCATGGAGACTGGACTG | | | |
| | CTGCTGCCACGAGCAACCGCA | | | |
| | CAGGATGCTGGAAAATACTATT | | | |
| | GCCACCGGGGCAATCTGACAA | | | |
| | TGTCCTTCCATCTGGAGATCAC | | | |
| | TGCAAGGCCCGTGCTGTGGCA | | | |
| | CTGGCTGCTGCGAACCGGAGG | | | |
| | ATGGAAGGTCAGTGCTGTGAC | | | |
| | ACTGGCATATCTGATCTTTTGC | | | |
| | CTGTGCTCCCTGGTGGGCATT | | | |
| | CTGCATCTGCAGAGAGCCCTG | | | |
| | GTGCTGCGGAGAAAGAGAAAG | | | |
| | AGAATGACTGACCCAACAAGA | | | |
| | AGGTTTTGA | | | |

pBP0607-pSFG-k-iMC.2A-ΔCD19

| | | SEQ ID | | SEQ ID |
|---|---|---|---|---|
| Fragment | Nucleotide | NO: | Peptide | NO: |
| Myr | ATGGGGAGTAGCAAGAGCAAG | 769 | MGSSKSKPKDPSQR | 770 |
| | CCTAAGGACCCCAGCCAGCGC | | | |
| Linker | CTCGAC | 771 | LN | 772 |
| MyD88 | ATGGCTGCAGGAGGTCCCGGC | 773 | MAAGGPGAGSAAPVSSTSSLPLA | 774 |
| | GCGGGGTCTGCGGCCCCGGT | | ALNMRVRRRLSLFLNVRTQVAAD | |
| | CTCCTCCACATCCTCCCTTCCC | | WTALAEEMDFEYLEIRQLETQAD | |
| | CTGGCTGCTCTCAACATGCGA | | PTGRLLDAWQGRPGASVGRLLD | |
| | GTGCGGCGCCGCCTGTCTCTG | | LLTKLGRDDVLLELGPSIEEDCQK | |
| | TTCTTGAACGTGCGGACACAG | | YILKQQQEEAEKPLQVAAVDSSV | |
| | GTGGCGGCCGACTGGACCGC | | PRTAELAGITTLDDPLGHMPERF | |
| | GCTGGCGGAGGAGATGGACTT | | DAFICYCPSDI | |
| | TGAGTACTTGGAGATCCGGCA | | | |
| | ACTGGAGACACAAGCGGACCC | | | |
| | CACTGGCAGGCTGCTGGACGC | | | |
| | CTGGCAGGGACGCCCTGGCG | | | |
| | CCTCTGTAGGCCGACTGCTCG | | | |
| | ATCTGCTTACCAAGCTGGGCC | | | |
| | GCGACGACGTGCTGCTGGAGC | | | |
| | TGGGACCCAGCATTGAGGAGG | | | |
| | ATTGCCAAAAGTATATCTTGAA | | | |
| | GCAGCAGCAGGAGGAGGCTG | | | |
| | AGAAGCCTTTACAGGTGGCCG | | | |
| | CTGTAGACAGCAGTGTCCCAC | | | |
| | GGACAGCAGAGCTGGCGGGC | | | |
| | ATCACCACACTTGATGACCCC | | | |
| | CTGGGGCATATGCCTGAGCGT | | | |
| | TTCGATGCCTTCATCTGCTATT | | | |
| | GCCCCAGCGACATC | | | |
| Linker | GTCGAG | 775 | VG | 776 |
| hCD40 | AAAAAGGTGGCCAAGAAGCCA | 777 | KKVAKKPTNKAPHPKQEPQEINF | 778 |
| | ACCAATAAGGCCCCCCACCCC | | PDDLPGSNTAAPVQETLHGCQP | |
| | AAGCAGGAGCCCCAGGAGATC | | VTQEDGKESRISVQERQ | |
| | AATTTTCCCGACGATCTTCCTG | | | |
| | GCTCCAACACTGCTGCTCCAG | | | |
| | TGCAGGAGACTTTACATGGAT | | | |
| | GCCAACCGGTCACCCAGGAGG | | | |
| | ATGGCAAAGAGAGTCGCATCT | | | |
| | CAGTGCAGGAGAGACAG | | | |
| Linker | GTCGAG | 779 | VG | 780 |
| Fv′ | GGCGTCCAAGTCGAAACCATT | 781 | GVQVETISPGDGRTFPKRGQTCV | 782 |
| | AGTCCCGGCGATGGCAGAACA | | VHYTGMLEDGKKVDSSRDRNKP | |
| | TTTCCTAAAAGGGGACAAACAT | | FKFMLGKQEVIRGWEEGVAQMS | |
| | GTGTCGTCCATTATACAGGCAT | | VGQRAKLTISPDYAYGATGHPGII | |
| | GTTGGAGGACGGCAAAAAGGT | | PPHATLVFDVELLKLE | |
| | GGACAGTAGTAGAGATCGCAA | | | |
| | TAAACCTTTCAAATTCATGTTG | | | |
| | GGAAAACAAGAAGTCATTAGG | | | |
| | GGATGGGAGGAGGGCGTGGC | | | |
| | TCAAATGTCCGTCGGCCAACG | | | |
| | CGCTAAGCTCACCATCAGCCC | | | |
| | CGACTACGCATACGGCGCTAC | | | |
| | CGGACATCCCGGAATTATTCC | | | |
| | CCCTCACGCTACCTTGGTGTTT | | | |
| | GACGTCGAACTGTTGAAGCTC | | | |
| | GAA | | | |
| Linker | GTCGAG | 783 | VG | 784 |
| Fv | GGAGTGCAGGTGGAGACTATC | 785 | GVQVETISPGDGRTFPKRGQTCV | 786 |
| | TCCCCAGGAGACGGGCGCAC | | VHYTGMLEDGKKVDSSRDRNKP | |
| | CTTCCCCAAGCGCGGCCAGAC | | FKFMLGKQEVIRGWEEGVAQMS | |
| | CTGCGTGGTGCACTACACCGG | | VGQRAKLTISPDYAYGATGHPGII | |
| | GATGCTTGAAGATGGAAAGAA | | PPHATLVFDVELLKLE | |
| | AGTTGATTCCTCCCGGGACAG | | | |
| | AAACAAGCCCTTTAAGTTTATG | | | |
| | CTAGGCAAGCAGGAGGTGATC | | | |
| | CGAGGCTGGGAAGAAGGGGTT | | | |
| | GCCCAGATGAGTGTGGGTCAG | | | |
| | AGAGCCAAACTGACTATATCTC | | | |
| | CAGATTATGCCTATGGTGCCAC | | | |
| | TGGGCACCCAGGCATCATCCC | | | |
| | ACCACATGCCACTCTCGTCTTC | | | |
| | GATGTGGAGCTTCTAAAACTG | | | |
| | GAA | | | |
| Linker | CCGCGG | 787 | PR | 788 |
| T2A | GAAGGCCGAGGGAGCCTGCT | 789 | EGRGSLLTCGDVEENPGP | 790 |
| | GACATGTGGCGATGTGGAGGA | | | |
| | AAACCCAGGACCA | | | |
| ΔCD19 | ATGCCACCACCTCGCCTGCTG | 791 | MPPPRLLFFLLFLTPMEVRPEEPL | 792 |
| | TTCTTTCTGCTGTTCCTGACAC | | VVKVEEGDNAVLQCLKGTSDGPT | |
| | CTATGGAGGTGCGACCTGAGG | | QQLTWSRESPLKPFLKLSLGLPG | |
| | AACCACTGGTCGTGAAGGTCG | | LGIHMRPLAIWLFIFNVSQQMGGF | |
| | AGGAAGGCGACAATGCCGTGC | | YLCQPGPPSEKAWQPGWTVNVE | |
| | TGCAGTGCCTGAAAGGCACTT | | GSGELFRWNVSDLGGLGCGLKN | |
| | CTGATGGGCCAACTCAGCAGC | | RSSEGPSSPSGKLMSPKLYVWA | |
| | TGACCTGGTCCAGGGAGTCTC | | KDRPEIWEGEPPCLPPRDSLNQS | |
| | CCCTGAAGCCTTTTCTGAAACT | | LSQDLTMAPGSTLWLSCGVPPD | |
| | GAGCCTGGGACTGCCAGGACT | | SVSRGPLSWTHVHPKGPKSLLSL | |
| | GGGAATCCACATGCGCCCTCT | | ELKDDRPARDMWVMETGLLLPR | |
| | GGCTATCTGGCTGTTCATCTTC | | ATAQDAGKYYCHRGNLTMSFHL | |
| | AACGTGAGCCAGCAGATGGGA | | EITARPVLWHWLLRTGGWKVSA | |
| | GGATTCTACCTGTGCCAGCCA | | VTLAYLIFCLCSLVGILHLQRALVL | |
| | GGACCACCATCCGAGAAGGCC | | RRKRKRMTDPTRRF* | |
| | TGGCAGCCTGGATGGACCGTC | | | |
| | AACGTGGAGGGGTCTGGAGAA | | | |
| | CTGTTTAGGTGGAATGTGAGT | | | |
| | GACCTGGGAGGACTGGGATGT | | | |
| | GGGCTGAAGAACCGCTCCTCT | | | |
| | GAAGGCCCAAGTTCACCCTCA | | | |
| | GGGAAGCTGATGAGCCCAAAA | | | |
| | CTGTACGTGTGGGCCAAAGAT | | | |
| | CGGCCCGAGATCTGGGAGGG | | | |
| | AGAACCTCCATGCCTGCCACC | | | |
| | TAGAGACAGCCTGAATCAGAG | | | |
| | TCTGTCACAGGATCTGACAATG | | | |
| | GCCCCCGGGTCCACTCTGTGG | | | |
| | CTGTCTTGTGGAGTCCCACCC | | | |
| | GACAGCGTGTCCAGAGGCCCT | | | |
| | CTGTCCTGGACCCACGTGCAT | | | |
| | CCTAAGGGGCCAAAAAGTCTG | | | |
| | CTGTCACTGGAACTGAAGGAC | | | |
| | GATCGGCCTGCCAGAGACATG | | | |
| | TGGGTCATGGAGACTGGACTG | | | |
| | CTGCTGCCACGAGCAACCGCA | | | |
| | CAGGATGCTGGAAAATACTATT | | | |
| | GCCACCGGGGCAATCTGACAA | | | |
| | TGTCCTTCCATCTGGAGATCAC | | | |
| | TGCAAGGCCCGTGCTGTGGCA | | | |
| | CTGGCTGCTGCGAACCGGAGG | | | |
| | ATGGAAGGTCAGTGCTGTGAC | | | |
| | ACTGGCATATCTGATCTTTTGC | | | |
| | CTGTGCTCCCTGGTGGGCATT | | | |
| | CTGCATCTGCAGAGAGCCCTG | | | |
| | GTGCTGCGGAGAAAGAGAAAG | | | |
| | AGAATGACTGACCCAACAAGA | | | |
| | AGGTTTTGA | | | |

pBP0668-pSFG-FRB_{l×2}.Caspase9.2A-Q.8stm.CD3zeta

| | | SEQ | | SEQ |
|---|---|---|---|---|
| | | ID | | ID |
| Fragment | Nucleotide | NO: | Peptide | NO: |
| FRB_{I}_{^{′}} | TGGCATGAAGGCCTGGAAGAG | 793 | WHEGLEEASRLYFGERNVKGMFE | 794 |
| | GCATCTCGTTTGTACTTTGGGG | | VLEPLHAMMERGPQTLKETSFNQ | |
| | AAAGGAACGTGAAAGGCATGT | | AYGRDLMEAQEWCRKYMKSGNV | |
| | TTGAGGTGCTGGAGCCCTTGC | | KDLLQAWDLYYHVFRRISK | |
| | ACGCTATGATGGAACGGGGCC | | | |
| | CCCAGACTCTGAAGGAAACAT | | | |
| | CCTTTAATCAGGCCTATGGTCG | | | |
| | AGATTTAATGGAGGCCCAAGA | | | |
| | GTGGTGCAGGAAGTACATGAA | | | |
| | ATCAGGGAATGTCAAGGACCT | | | |
| | CCTCCAAGCCTGGGACCTCTA | | | |
| | TTATCATGTGTTCCGACGAATC | | | |
| | TCAAAG | | | |
| Linker | GTCGAG | 795 | VG | 796 |
| FRB_{I} | TGGCATGAAGGGTTGGAAGAA | 797 | WHEGLEEASRLYFGERNVKGMFE | 798 |
| | GCTTCAAGGCTGTACTTCGGA | | VLEPLHAMMERGPQTLKETSFNQ | |
| | GAGAGGAACGTGAAGGGCATG | | AYGRDLMEAQEWCRKYMKSGNV | |
| | TTTGAGGTTCTTGAACCTCTGC | | KDLLQAWDLYYHVFRRISK | |
| | ACGCCATGATGGAACGGGGAC | | | |
| | CGCAGACACTGAAAGAAACCT | | | |
| | CTTTTAATCAGGCCTACGGCAG | | | |
| | AGACCTGATGGAGGCCCAAGA | | | |
| | ATGGTGTAGAAAGTATATGAAA | | | |
| | TCCGGTAACGTGAAAGACCTG | | | |
| | CTCCAGGCCTGGGACCTTTATT | | | |
| | ACCATGTGTTCAGGCGGATCA | | | |
| | GTAAG | | | |
| Linker | TCAGGCGGTGGCTCAGGT | 799 | SGGGSG | 800 |
| ΔCaspase9 | TCGACGGATTTGGTGATGTCG | 801 | DGFGDVGALESLRGNADLAYILSM | 802 |
| | GTGCTCTTGAGAGTTTGAGGG | | EPCGHCLIINNVNFCRESGLRTRT | |
| | GAAATGCAGATTTGGCTTACAT | | GSNIDCEKLRRRFSSLHFMVEVKG | |
| | CCTGAGCATGGAGCCCTGTGG | | DLTAKKMVLALLELARQDHGALDC | |
| | CCACTGCCTCATTATCAACAAT | | CVVVILSHGCQASHLQFPGAVYGT | |
| | GTGAACTTCTGCCGTGAGTCC | | DGCPVSVEKIVNIFNGTSCPSLGG | |
| | GGGCTCCGCACCCGCACTGGC | | KPKLFFIQACGGEQKDHGFEVAST | |
| | TCCAACATCGACTGTGAGAAG | | SPEDESPGSNPEPDATPFQEGLR | |
| | TTGCGGCGTCGCTTCTCCTCG | | TFDQLDAISSLPTPSDIFVSYSTFP | |
| | CTGCATTTCATGGTGGAGGTG | | GFVSVVRDPKSGSVVYVETLDDIFE | |
| | AAGGGCGACCTGACTGCCAAG | | QWAHSEDLQSLLLRVANAVSVKGI | |
| | AAAATGGTGCTGGCTTTGCTG | | YKQMPGCFNFLRKKLFFKTSASRA | |
| | GAGCTGGCGCGGCAGGACCA | | | |
| | CGGTGCTCTGGACTGCTGCGT | | | |
| | GGTGGTCATTCTCTCTCACGG | | | |
| | CTGTCAGGCCAGCCACCTGCA | | | |
| | GTTCCCAGGGGCTGTCTACGG | | | |
| | CACAGATGGATGCCCTGTGTC | | | |
| | GGTCGAGAAGATTGTGAACAT | | | |
| | CTTCAATGGGACCAGCTGCCC | | | |
| | CAGCCTGGGAGGGAAGCCCAA | | | |
| | GCTCTTTTTCATCCAGGCCTGT | | | |
| | GGTGGGGAGCAGAAAGACCAT | | | |
| | GGGTTTGAGGTGGCCTCCACT | | | |
| | TCCCCTGAAGACGAGTCCCCT | | | |
| | GGCAGTAACCCCGAGCCAGAT | | | |
| | GCCACCCCGTTCCAGGAAGGT | | | |
| | TTGAGGACCTTCGACCAGCTG | | | |
| | GACGCCATATCTAGTTTGCCCA | | | |
| | CACCCAGTGACATCTTTGTGTC | | | |
| | CTACTCTACTTTCCCAGGTTTT | | | |
| | GTTTCCTGGAGGGACCCCAAG | | | |
| | AGTGGCTCCTGGTACGTTGAG | | | |
| | ACCCTGGACGACATCTTTGAG | | | |
| | CAGTGGGCTCACTCTGAAGAC | | | |
| | CTGCAGTCCCTCCTGCTTAGG | | | |
| | GTCGCTAATGCTGTTTCGGTGA | | | |
| | AAGGGATTTATAAACAGATGCC | | | |
| | TGGTTGCTTTAATTTCCTCCGG | | | |
| | AAAAAACTTTTCTTTAAAACATC | | | |
| | AGCTAGCAGAGCC | | | |
| Linker | CCGCGG | 803 | PR | 804 |
| T2A | GAAGGCCGAGGGAGCCTGCT | 805 | EGRGSLLTCGDVEENPGP | 806 |
| | GACATGTGGCGATGTGGAGGA | | | |
| | AAACCCAGGACCA | | | |
| Signal | ATGGAATTTGGCCTCTCCTGGT | 807 | MEFGLSVVLFLVAILKGVQCSR | 808 |
| Peptide | TGTTTCTCGTGGCCATTCTTAA | | | |
| | GGGTGTGCAGTGCTCCAGA | | | |
| Linker | ATGCAT | 809 | MH | 810 |
| Q-Bend | GAACTTCCTACTCAGGGGACTT | 811 | ELPTQGTFSNVSTNVS | 812 |
| (CD34 | TCTCAAACGTTAGCACAAACGT | | | |
| Epitope) | AAGT | | | |
| CD8 | CCCGCCCCAAGACCCCCCACA | 813 | PAPRPPTPAPTIASQPLSLRPEAC | 814 |
| Stalk | CCTGCGCCGACCATTGCTTCT | | RPAAGGAVHTRGLDFACD | |
| | CAACCCCTGAGTTTGAGACCC | | | |
| | GAGGCCTGCCGGCCAGCTGC | | | |
| | CGGCGGGGCCGTGCATACAA | | | |
| | GAGGACTCGATTTCGCTTGCG | | | |
| | AC | | | |
| CD8a tm | ATCTATATCTGGGCACCTCTCG | 815 | IYIWAPLAGTCGVLLLSLVITLYCNH | 816 |
| | CTGGCACCTGTGGAGTCCTTC | | RNRRRVCKCPRVD | |
| | TGCTCAGCCTGGTTATTACTCT | | | |
| | GTACTGTAATCACCGGAATCG | | | |
| | CCGCCGCGTTTGTAAGTGTCC | | | |
| | CAGGGTCGAC | | | |
| CD3 zeta | AGAGTGAAGTTCAGCAGGAGC | 817 | RVKFSRSADAPAYQQGQNQLYNE | 818 |
| | GCAGACGCCCCCGCGTACCAG | | LNLGRREEYDVLDKRRGRDPEMG | |
| | CAGGGCCAGAACCAGCTCTAT | | GKPRRKNPQEGLYNELQKDKMAE | |
| | AACGAGCTCAATCTAGGACGA | | AYSEIGMKGERRRGKGHDGLYQG | |
| | AGAGAGGAGTACGATGTTTTG | | LSTATKDTYDALHMQALPP | |
| | GACAAGAGACGTGGCCGGGA | | | |
| | CCCTGAGATGGGGGGAAAGCC | | | |
| | GAGAAGGAAGAACCCTCAGGA | | | |
| | AGGCCTGTACAATGAACTGCA | | | |
| | GAAAGATAAGATGGCGGAGGC | | | |
| | CTACAGTGAGATTGGGATGAA | | | |
| | AGGCGAGCGCCGGAGGGGCA | | | |
| | AGGGGCACGATGGCCTTTACC | | | |
| | AGGGTCTCAGTACAGCCACCA | | | |
| | AGGACACCTACGACGCCCTTC | | | |
| | ACATGCAAGCTCTTCCACCTCG | | | |

pBP0608-pSFG-ΔMyriMC.2A-ΔCD19.Q.8stm.CD3zeta

| | | SEQ | | SEQ |
|---|---|---|---|---|
| Fragment | Nucleotide | ID NO: | Peptide | ID NO: |
| MyD88 | ATGGCTGCAGGAGGTCCCGGCG | 819 | MAAGGPGAGSAAPVSSTSSLPLA | 820 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQADP | |
| | CTGCTCTCAACATGCGAGTGCGG | | TGRLLDAWQGRPGASVGRLLDLL | |
| | CGCCGCCTGTCTCTGTTCTTGAA | | TKLGRDDVLLELGPSIEEDCQKYIL | |
| | CGTGCGGACACAGGTGGCGGCC | | KQQQEEAEKPLQVAAVDSSVPRT | |
| | GACTGGACCGCGCTGGCGGAGG | | AELAGITTLDDPLGHMPERFDAFIC | |
| | AGATGGACTTTGAGTACTTGGAG | | YCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 821 | VE | 822 |
| hCD40 | AAAAAGGTGGCCAAGAAGCCAAC | 823 | KKVAKKPTNKAPHPKQEPQEINFP | 824 |
| | CAATAAGGCCCCCCACCCCAAGC | | DDLPGSNTAAPVQETLHGCQPVT | |
| | AGGAGCCCCAGGAGATCAATTTT | | QEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 825 | VE | 826 |
| Fv′ | GGCGTCCAAGTCGAAACCATTAG | 827 | GVQVETISPGDGRTFPKRGQTCV | 828 |
| | TCCCGGCGATGGCAGAACATTTC | | VHYTGMLEDGKKVDSSRDRNKPF | |
| | CTAAAAGGGGACAAACATGTGTC | | KFMLGKQEVIRGVVEEGVAQMSVG | |
| | GTCCATTATACAGGCATGTTGGA | | QRAKLTISPDYAYGATGHPGIIPPH | |
| | GGACGGCAAAAAGGTGGACAGTA | | ATLVFDVELLKLE | |
| | GTAGAGATCGCAATAAACCTTTCA | | | |
| | AATTCATGTTGGGAAAACAAGAAG | | | |
| | TCATTAGGGGATGGGAGGAGGGC | | | |
| | GTGGCTCAAATGTCCGTCGGCCA | | | |
| | ACGCGCTAAGCTCACCATCAGCC | | | |
| | CCGACTACGCATACGGCGCTACC | | | |
| | GGACATCCCGGAATTATTCCCCC | | | |
| | TCACGCTACCTTGGTGTTTGACGT | | | |
| | CGAACTGTTGAAGCTCGAA | | | |
| Linker | GTCGAG | 829 | VE | 830 |
| Fv | GGAGTGCAGGTGGAGACTATCTC | 831 | GVQVETISPGDGRTFPKRGQTCV | 832 |
| | CCCAGGAGACGGGCGCACCTTCC | | VHYTGMLEDGKKVDSSRDRNKPF | |
| | CCAAGCGCGGCCAGACCTGCGT | | KFMLGKQEVIRGVVEEGVAQMSVG | |
| | GGTGCACTACACCGGGATGCTTG | | QRAKLTISPDYAYGATGHPGIIPPH | |
| | AAGATGGAAAGAAAGTTGATTCCT | | ATLVFDVELLKLE | |
| | CCCGGGACAGAAACAAGCCCTTT | | | |
| | AAGTTTATGCTAGGCAAGCAGGA | | | |
| | GGTGATCCGAGGCTGGGAAGAAG | | | |
| | GGGTTGCCCAGATGAGTGTGGGT | | | |
| | CAGAGAGCCAAACTGACTATATCT | | | |
| | CCAGATTATGCCTATGGTGCCACT | | | |
| | GGGCACCCAGGCATCATCCCACC | | | |
| | ACATGCCACTCTCGTCTTCGATGT | | | |
| | GGAGCTTCTAAAACTGGAA | | | |
| Linker | CCGCGG | 833 | PR | 834 |
| T2A | GAAGGCCGAGGGAGCCTGCTGA | 835 | EGRGSLLTCGDVEENPGP | 836 |
| | CATGTGGCGATGTGGAGGAAAAC | | | |
| | CCAGGACCA | | | |
| Linker | CCATGG | 837 | PW | 838 |
| Signal | ATGGAGTTTGGACTTTCTTGGTTG | 839 | MEFGLSVVLFLVAILKGVQCSR | 840 |
| Peptide | TTTTTGGTGGCAATTCTGAAGGGT | | | |
| | GTCCAGTGTAGCAGG | | | |
| FMC63-VL | GACATCCAGATGACACAGACTAC | 841 | DIQMTQTTSSLSASLGDRVTISCR | 842 |
| | ATCCTCCCTGTCTGCCTCTCTGG | | ASQDISKYLNVVYQQKPDGTVKLLI | |
| | GAGACAGAGTCACCATCAGTTGC | | YHTSRLHSGVPSRFSGSGSGTDY | |
| | AGGGCAAGTCAGGACATTAGTAA | | SLTISNLEQEDIATYFCQQGNTLPY | |
| | ATATTTAAATTGGTATCAGCAGAA | | TFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 843 | GGGSGGGG | 844 |
| Linker | GC | | | |
| FMC63-VH | GAGGTGAAACTGCAGGAGTCAGG | 845 | EVKLQESGPGLVAPSQSLSVTCTV | 846 |
| | ACCTGGCCTGGTGGCGCCCTCAC | | SGVSLPDYGVSWIRQPPRKGLEW | |
| | AGAGCCTGTCCGTCACATGCACT | | LGVIWGSETTYYNSALKSRLTIIKD | |
| | GTCTCAGGGGTCTCATTACCCGA | | NSKSQVFLKMNSLQTDDTAIYYCA | |
| | CTATGGTGTAAGCTGGATTCGCC | | KHYYYGGSYAMDYWGQGTSVTV | |
| | AGCCTCCACGAAAGGGTCTGGAG | | SS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 847 | GS | 848 |
| Q-Bend | GAACTTCCTACTCAGGGGACTTTC | 849 | ELPTQGTFSNVSTNVS | 850 |
| (CD34 | TCAAACGTTAGCACAAACGTAAGT | | | |
| Epitope) | | | | |
| CD8 Stalk | CCCGCCCCAAGACCCCCCACACC | 851 | PAPRPPTPAPTIASQPLSLRPEAC | 852 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8a tm | ATCTATATCTGGGCACCTCTCGCT | 853 | IYIWAPLAGTCGVLLLSLVITLYCNH | 854 |
| | GGCACCTGTGGAGTCCTTCTGCT | | RNRRRVCKCPR | |
| | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 855 | VD | 856 |
| CD3 zeta | AGAGTGAAGTTCAGCAGGAGCGC | 857 | RVKFSRSADAPAYQQGQNQLYNE | 858 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPP | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCG | | | |

pBP0609: pSFG-iMC.2A-ΔCD19.Q.8stm.CD3zeta

| | | SEQ | | SEQ |
|---|---|---|---|---|
| | | ID | | ID |
| Fragment | Nucleotide | NO: | Peptide | NO: |
| Myr | ATGGGGAGTAGCAAGAGCAAGCC | 859 | MGSSKSKPKDPSQR | 860 |
| | TAAGGACCCCAGCCAGCGC | | | |
| Linker | CTCGAC | 861 | LD | 862 |
| MyD88 | ATGGCTGCAGGAGGTCCCGGCG | 863 | MAAGGPGAGSAAPVSSTSSLPLA | 864 |
| | CGGGGTCTGCGGCCCCGGTCTC | | ALNMRVRRRLSLFLNVRTQVAAD | |
| | CTCCACATCCTCCCTTCCCCTGG | | VVTALAEEMDFEYLEIRQLETQADP | |
| | CTGCTCTCAACATGCGAGTGCGG | | TGRLLDAWQGRPGASVGRLLDLL | |
| | CGCCGCCTGTCTCTGTTCTTGAA | | TKLGRDDVLLELGPSIEEDCQKYIL | |
| | CGTGCGGACACAGGTGGCGGCC | | KQQQEEAEKPLQVAAVDSSVPRT | |
| | GACTGGACCGCGCTGGCGGAGG | | AELAGITTLDDPLGHMPERFDAFIC | |
| | AGATGGACTTTGAGTACTTGGAG | | YCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 865 | VE | 866 |
| hCD40 | AAAAAGGTGGCCAAGAAGCCAAC | 867 | KKVAKKPTNKAPHPKQEPQEINFP | 868 |
| | CAATAAGGCCCCCCACCCCAAGC | | DDLPGSNTAAPVQETLHGCQPVT | |
| | AGGAGCCCCAGGAGATCAATTTT | | QEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 869 | VE | 870 |
| Fv′ | GGCGTCCAAGTCGAAACCATTAG | 871 | GVQVETISPGDGRTFPKRGQTCV | 872 |
| | TCCCGGCGATGGCAGAACATTTC | | VHYTGMLEDGKKVDSSRDRNKPF | |
| | CTAAAAGGGGACAAACATGTGTC | | KFMLGKQEVIRGVVEEGVAQMSVG | |
| | GTCCATTATACAGGCATGTTGGA | | QRAKLTISPDYAYGATGHPGIIPPH | |
| | GGACGGCAAAAAGGTGGACAGTA | | ATLVFDVELLKLE | |
| | GTAGAGATCGCAATAAACCTTTCA | | | |
| | AATTCATGTTGGGAAAACAAGAAG | | | |
| | TCATTAGGGGATGGGAGGAGGGC | | | |
| | GTGGCTCAAATGTCCGTCGGCCA | | | |
| | ACGCGCTAAGCTCACCATCAGCC | | | |
| | CCGACTACGCATACGGCGCTACC | | | |
| | GGACATCCCGGAATTATTCCCCC | | | |
| | TCACGCTACCTTGGTGTTTGACGT | | | |
| | CGAACTGTTGAAGCTCGAA | | | |
| Linker | GTCGAG | 873 | VE | 874 |
| Fv | GGAGTGCAGGTGGAGACTATCTC | 875 | GVQVETISPGDGRTFPKRGQTCV | 876 |
| | CCCAGGAGACGGGCGCACCTTCC | | VHYTGMLEDGKKVDSSRDRNKPF | |
| | CCAAGCGCGGCCAGACCTGCGT | | KFMLGKQEVIRGVVEEGVAQMSVG | |
| | GGTGCACTACACCGGGATGCTTG | | QRAKLTISPDYAYGATGHPGIIPPH | |
| | AAGATGGAAAGAAAGTTGATTCCT | | ATLVFDVELLKLE | |
| | CCCGGGACAGAAACAAGCCCTTT | | | |
| | AAGTTTATGCTAGGCAAGCAGGA | | | |
| | GGTGATCCGAGGCTGGGAAGAAG | | | |
| | GGGTTGCCCAGATGAGTGTGGGT | | | |
| | CAGAGAGCCAAACTGACTATATCT | | | |
| | CCAGATTATGCCTATGGTGCCACT | | | |
| | GGGCACCCAGGCATCATCCCACC | | | |
| | ACATGCCACTCTCGTCTTCGATGT | | | |
| | GGAGCTTCTAAAACTGGAA | | | |
| Linker | CCGCGG | 877 | PR | 878 |
| T2A | GAAGGCCGAGGGAGCCTGCTGA | 879 | EGRGSLLTCGDVEENPGP | 880 |
| | CATGTGGCGATGTGGAGGAAAAC | | | |
| | CCAGGACCA | | | |
| Linker | CCATGG | 881 | PW | 882 |
| Signal | ATGGAGTTTGGACTTTCTTGGTTG | 883 | MEFGLSVVLFLVAILKGVQCSR | 884 |
| Peptide | TTTTTGGTGGCAATTCTGAAGGGT | | | |
| | GTCCAGTGTAGCAGG | | | |
| FMC63-VL | GACATCCAGATGACACAGACTAC | 885 | DIQMTQTTSSLSASLGDRVTISCR | 886 |
| | ATCCTCCCTGTCTGCCTCTCTGG | | ASQDISKYLNVVYQQKPDGTVKLLI | |
| | GAGACAGAGTCACCATCAGTTGC | | YHTSRLHSGVPSRFSGSGSGTDY | |
| | AGGGCAAGTCAGGACATTAGTAA | | SLTISNLEQEDIATYFCQQGNTLPY | |
| | ATATTTAAATTGGTATCAGCAGAA | | TFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 887 | GGGSGGGG | 888 |
| Linker | GC | | | |
| FMC63-VH | GAGGTGAAACTGCAGGAGTCAGG | 889 | EVKLQESGPGLVAPSQSLSVTCTV | 890 |
| | ACCTGGCCTGGTGGCGCCCTCAC | | SGVSLPDYGVSWIRQPPRKGLEW | |
| | AGAGCCTGTCCGTCACATGCACT | | LGVIWGSETTYYNSALKSRLTIIKD | |
| | GTCTCAGGGGTCTCATTACCCGA | | NSKSQVFLKMNSLQTDDTAIYYCA | |
| | CTATGGTGTAAGCTGGATTCGCC | | KHYYYGGSYAMDYWGQGTSVTV | |
| | AGCCTCCACGAAAGGGTCTGGAG | | SS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 891 | GS | 892 |
| Q-Bend | GAACTTCCTACTCAGGGGACTTTC | 893 | ELPTQGTFSNVSTNVS | 894 |
| (CD34 | TCAAACGTTAGCACAAACGTAAGT | | | |
| Epitope) | | | | |
| CD8 Stalk | CCCGCCCCAAGACCCCCCACACC | 895 | PAPRPPTPAPTIASQPLSLRPEAC | 896 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8a tm | ATCTATATCTGGGCACCTCTCGCT | 897 | IYIWAPLAGTCGVLLLSLVITLYCNH | 898 |
| | GGCACCTGTGGAGTCCTTCTGCT | | RNRRRVCKCPR | |
| | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 899 | VD | 900 |
| CD3 zeta | AGAGTGAAGTTCAGCAGGAGCGC | 901 | RVKFSRSADAPAYQQGQNQLYNE | 902 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPP | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCG | | | |

### Example 24: An Inducible Cell Death Switch Directed by Heterodimerizing Ligands

### Methods

Transfection of Cells

HEK 293T cells (5×10⁵) were seeded on a 100-mm tissue culture dish in 10 mL DMEM4500, supplemented with glutamine, penicillin/streptomycin and 10% fetal calf serum. After 16-30 hours incubation, cells were transfected using Novagen's GeneJuice® protocol. Briefly, for each transfection, 0.5 mL OptiMEM was pipetted into a 1.5-mL microcentrifuge tube and 15 μL GeneJuice reagent added followed by 5 sec. vortexing. Samples were rested 5 minutes to settle the GeneJuice suspension. DNA (5 μg total) was added to each tube and mixed by pipetting up and down four times. Samples were allowed to rest for 5 minutes for GeneJuice-DNA complex formation and the suspension added dropwise to one dish of 293T cells. A typical transfection contains 1 μg SRα-SEAP (pBP0046) (3), 2 μg FRB-Caspase-9 (pBP0463) and 2 μg FKBPv12-Caspase-9 (pBP0044) (7).

Stimulation of Cells with Dimerizing Drugs

24 hours following transfection (4.1), 293T cells were split to 96-well plates and incubated with dilutions of dimerizing drugs. Briefly, 100 μL media was added to each well of a 96-well flat-bottom plate. Drugs were diluted in tubes to a concentration 4× the top concentration in the gradient to be place on the plate. 100 μL of dimerizing ligand (rimiducid, rapamycin, isopropoxylrapamycin) was added to each of three wells on the far right of the plate (assays are thereby performed in triplicate). 100 μL from each drug-containing well was then transferred to the adjacent well and the cycle repeated 10 times to produce a serial two-fold step gradient. The last wells were untreated and serve as a control for basal reporter activity. Transfected 293 cells were then trypsinized, washed with complete media, suspended in media and 100 μL aliquoted to each well containing drug (or no drug). Cells were incubated 24 hours.

### Assay of Reporter Activity

The SRα promoter is a hybrid transcriptional element comprising the SV40 early region (which drives T antigen transcription) and parts (R and U5) of the Long Terminal Repeat (LTR) of Human T Cell Lymphotropic Virus (HTLV-1). This promoter drives high, constitutive levels of the Secreted Alkaline Phosphate (SeAP) reporter gene. Activation of caspase-9 by dimerization rapidly leads to cell death and the proportion of cells dying increases with increasing drug amounts. When cells die, transcription and translation of reporter stops but already secreted reporter proteins persists in the media. Loss of constitutive SeAP activity is thereby an effective proxy for drug-dependent activation of cell death.

24 hours after drug stimulation, 96-well plates were wrapped to prevent evaporation and incubated at 65° C. for 2 hours to inactivate endogenous and serum phosphatases while the heat-stable SeAP reporter remains (1, 4, 12). 100 μL samples from each well were loaded into individual wells of a 96-well assay plate with black sides. Samples were incubated with 0.5 mM 4-methylumbelliferyl phosphate (4-MUP) in 0.5 M diethanolamine at pH 10.0 for 4 to 16 hours. Phosphatase activity was measured by fluorescence with excitation at 355 nm and emission at 460 nm. Data was transferred to a Microsoft Excel spreadsheet for tabulation and graphed with GraphPad Prism.

### Production of Isopropyloxyrapamycin

The method of Luengo et al. ((J. Org. Chem 59:6512, (1994)), (16, 17)) was employed. Briefly, 20 mg of rapamycin was dissolved in in 3 mL isopropanol and 22.1 mg of p-toluene sulfonic acid was added and incubated at room temperature with stirring for 4-12 hours. At completion, 5 mL ethyl acetate was added and products were extracted five times with saturated sodium bicarbonate and 3 times with brine (saturated sodium chloride). The organic phase was dried and redissolved in ethyl acetate: hexane (3:1). Stereoisomers and minor products were resolved by FLASH chromatography on a 10 to 15-mL silica gel column with 3:1 ethyl acetate: hexane under 3-4 KPa pressure and fractions dried. Fractions were assayed by spectrophotometry at 237 nM, 267 nM, 278 nM and 290 nM and tested for binding specificity in a FRB allele-specific transcriptional switch.

Direct Dimerization of FRB-Caspase with FKBP-Caspase with Rapamycin Directs Apoptosis.

Dimerization of FKBP-fused caspases can be dimerized by homodimerizer molecules, such as AP1510, AP20187 or AP1903. A similar pro-apoptotic switch can be directed via heterodimerization of a binary switch using rapamycin by coexpression of a FRB-Caspase-9 fusion protein along with FKBP-Caspase-9, leading to homodimerization of the caspase domains. In FIG. 37, a constitutively active SeAP reporter plasmid was cotransfected into 293T cells along with the caspase constructs. Transfected cells abundantly produced SeAP that was readily measured without drug and which served as the 100% normalization standard in the experiment. Incubation of the two fusion proteins with rimiducid produces a dose-dependent homodimerization of only FKBP12-Caspase9, leading to dimerization and activation of apoptosis, while FRB-Caspase9 remains excluded from the rimiducid-driven complex (left). In contrast, incubation with rapamycin associates FRB and FKBP directly and linked Caspase-9 moieties associate and activate. Cell death was measured indirectly by the loss of SeAP reporter production as cells die. This experiment demonstrated that heterodimerization with rapamycin produces dose-dependent cell death, revealing a novel safety switch with nanomolar drug sensitivity.

FIG. 37—Drug induced programmed cell death by homodimerization or heterodimerization of tagged caspase 9. 293T cells were transfected with SRα-SeAP (pBP0046), pSH1-FKBPv12-Caspase9 (pBP0044) and pSH1-FRB_{L}-Caspase9 (pBP0463). After 24-hr incubation, cells were split and incubated with increasing concentrations of rapamycin (blue), rimiducid (red) or ethanol (the solvent containing stock rapamycin). Loss of reporter activity is a proxy for the loss of cell viability. Reporter activity is expressed as a percentage of the average of 8 control wells containing no drug. Assays with drugs were performed in triplicate.

### Cell Death can be Directed by Rapamycin or Rapamycin Analogs.

Rapamycin is an effective heterodimerizing agent, but as a result of causing the docking of FKBP12 with the protein kinase mTOR, rapamycin is also a potent inhibitor of signal transduction, resulting in reduced protein translation and reduced cell growth. Derivatives of rapamycin at C3 or C7 ring positions have reduced affinity for mTOR but retain high affinity for mutants in “helix 4” of the FRB domain. Plasmid pBP0463 contains a mutation that substitutes leucine for the wild-type threonine at position 2098 in the FRB domain (using the mTOR numbering) and accommodates derivatives at C7. Incubation of 293T cells transfected with FRB_{L}-Caspase 9, FKBP_{V}12-Caspase 9 and the constitutive SeAP reporter produced a dose-dependent high efficacy cell death switch with rapamycin or the rapamycin analog (rapalog) C7-isopropyloxyrapamycin (FIG. 38).

FIG. 38—Rapalog-induced cell death switch. 293T cells were transfected with SRα-SeAP (pBP0046), pSH1-FKBPv12-Caspase9 (pBP0044) and pSH1-FRB_{L}-Caspase9 (pBP0463). After 24-hr incubation, cells were split and incubated with increasing concentrations of rapamycin (blue), C7-isopropyloxyrapamycin (green) or ethanol (the solvent containing drug stocks). Loss of reporter activity is a proxy for loss of cell viability. Reporter activity is expressed as a percentage of the average of 8 wells containing no drug. Drug-containing assays were performed in triplicate.

### Rapamycin-Induced Cell Death Requires the Presence of FRB-Caspase-9.

To demonstrate that rapamycin-induced cell death results from dimerization of Caspase-9 molecules linked separately with FRB and FKBP12, two control experiments were performed

(FIGS. 39 and 40). iC9 (FKBPv12-Caspase-9) was cotransfected with a control vector expressing only an epitope tag (FIG. 39) or a vector containing FRB without caspase fusion, but instead with a short, irrelevant tag (FIG. 40). In each case, incubation with rimiducid effectively permitted homodimerization and induction of Caspase-9, but rapamycin incubation did not promote cell death. These findings support the conclusion that the mechanism of rapamycin/rapalog-mediated cell death is activation of dimerized C9 molecules rather than recruitment of mTOR to Caspase-9 or due to an indirect mechanism involving endogenous mTOR inhibition.

FIG. 39—FRB-Caspase-9 is required for a rapamycin-induced cell death switch. 293T cells were transfected with SRα-SeAP (pBP0046), pS-NLS-E and pSH1-FKBPv12-Caspase9 (pBP0044).

FIG. 40—Caspase-9 fusion with FRB is required for a rapamycin-induced cell death switch. 293T cells were transfected with SRα-SeAP (pBP0046), pSH1-FRB_{L}-VP16 (pBP0731) (4) and pSH1-FKBPv12-Caspase9 (pBP0044). After 24-hr incubation, cells were split and incubated with increasing concentrations of rapamycin (blue), C7-isopropyloxyrapamycin (red), rimiducid (green) or ethanol (the solvent containing drug stocks). Loss of reporter activity is a proxy for the loss of cell viability. Reporter activity is expressed as a percentage of the average of 8 wells containing no drug. Drug-containing wells were assayed in triplicate wells.

The following references are referred to in this Example and are hereby incorporated by reference herein in their entireties:
1. Spencer D M, Wandless T J, Schreiber S L, and Crabtree G R. Controlling signal transduction with synthetic ligands. Science. 1993; 262(5136):1019-24.
2. Acevedo V D, Gangula R D, Freeman K W, Li R, Zhang Y, Wang F, Ayala G E, Peterson L E, Ittmann M, and Spencer D M. Inducible FGFR-1 activation leads to irreversible prostate adenocarcinoma and an epithelial-to-mesenchymal transition. Cancer Cell. 2007; 12(6):559-71.
3. Spencer D M, Belshaw P J, Chen L, Ho S N, Randazzo F, Crabtree G R, and Schreiber S L. Functional analysis of Fas signaling in vivo using synthetic inducers of dimerization. Curr Biol. 1996; 6(7):839-47.
4. Bayle J H, Grimley J S, Stankunas K, Gestwicki J E, Wandless T J, and Crabtree G R. Rapamycin analogs with differential binding specificity permit orthogonal control of protein activity. Chem Biol. 2006; 13(1):99-107.
5. Strasser A, Cory S, and Adams J M. Deciphering the rules of programmed cell death to improve therapy of cancer and other diseases. EMBO J. 2011; 30(18):3667-83.
6. Fan L, Freeman K W, Khan T, Pham E, and Spencer D M. Improved artificial death switches based on caspases and FADD. Hum Gene Ther. 1999; 10(14):2273-85.
7. Straathof K C, Pule M A, Yotnda P, Dotti G, Vanin E F, Brenner M K, Heslop H E, Spencer D M, and Rooney C M. An inducible caspase 9 safety switch for T-cell therapy. Blood. 2005; 105(11):4247-54.
8. Sabatini D M, Erdjument-Bromage H, Lui M, Tempst P, and Snyder S H. RAFT1: a mammalian protein that binds to FKBP12 in a rapamycin-dependent fashion and is homologous to yeast TORs. Cell. 1994; 78(1):35-43.
9. Brown E J, Albers M W, Shin T B, Ichikawa K, Keith C T, Lane W S, and Schreiber S L. A mammalian protein targeted by G1-arresting rapamycin-receptor complex. Nature. 1994; 369(6483):756-8.
10. Chen J, Zheng X F, Brown E J, and Schreiber S L. Identification of an 11-kDa FKBP12-rapamycin-binding domain within the 289-kDa FKBP12-rapamycin-associated protein and characterization of a critical serine residue. Proc Natl Acad Sci USA. 1995; 92(11):4947-51.
11. Choi J, Chen J, Schreiber S L, and Clardy J. Structure of the FKBP12-rapamycin complex interacting with the binding domain of human FRAP. Science. 1996; 273(5272):239-42.
12. Ho S N, Biggar S R, Spencer D M, Schreiber S L, and Crabtree G R. Dimeric ligands define a role for transcriptional activation domains in reinitiation. Nature. 1996; 382(6594):822-6.
13. Klemm J D, Beals C R, and Crabtree G R. Rapid targeting of nuclear proteins to the cytoplasm. Curr Biol. 1997; 7(9):638-44.
14. Stankunas K, Bayle J H, Gestwicki J E, Lin Y M, Wandless T J, and Crabtree G R. Conditional protein alleles using knockin mice and a chemical inducer of dimerization. Mol Cell. 2003; 12(6):1615-24.
15. Stankunas K, Bayle J H, Havranek J J, Wandless T J, Baker D, Crabtree G R, and Gestwicki J E. Rescue of Degradation-Prone Mutants of the FK506-Rapamycin Binding (FRB) Protein with Chemical Ligands. Chembiochem. 2007.
16. Liberles S D, Diver S T, Austin D J, and Schreiber S L. Inducible gene expression and protein translocation using nontoxic ligands identified by a mammalian three-hybrid screen. Proc Natl Acad Sci USA. 1997; 94(15):7825-30.
17. Luengo J I, Yamashita D S, Dunnington D, Beck A K, Rozamus L W, Yen H K, Bossard M J, Levy M A, Hand A, Newman-Tarr T, et al. Structure-activity studies of rapamycin analogs: evidence that the C-7 methoxy group is part of the effector domain and positioned at the FKBP12-FRAP interface. Chem Biol. 1995; 2(7):471-81.
   pBP0463-pSH1-FRB_{L}.dCaspase9.T2A (From FIG. 41)

| | | SEQ | | SEQ |
|---|---|---|---|---|
| Fragment | Nucleotide | ID NO: | Peptide | ID NO: |
| Linker | ATGCTCGAG | 903 | MLE | 904 |
| FRB_{L} | TGGCATGAAGGGTTGGAAGAAGC | 905 | GVQVETISPGDGRTFPKRGQTCV | 906 |
| | TTCAAGGCTGTACTTCGGAGAGA | | VHYTGMLEDGKKFDSSRDRNKPF | |
| | GGAACGTGAAGGGCATGTTTGAG | | KFMLGKQEVIRGWEEGVAQMSVG | |
| | GTTCTTGAACCTCTGCACGCCAT | | QRAKLTISPDYAYGATGHPPKIPP | |
| | GATGGAACGGGGACCGCAGACA | | HATLVFDVELLKLE | |
| | CTGAAAGAAACCTCTTTTAATCAG | | | |
| | GCCTACGGCAGAGACCTGATGGA | | | |
| | GGCCCAAGAATGGTGTAGAAAGT | | | |
| | ATATGAAATCCGGTAACGTGAAAG | | | |
| | ACCTGCTCCAGGCCTGGGACCTT | | | |
| | TATTACCATGTGTTCAGGCGGATC | | | |
| | AGTAAG | | | |
| Linker | TCAGGCGGTGGCTCAGGTGTCGA | 907 | SGGGSGVD | 908 |
| | G | | | |
| Δ- | GTCGACGGATTTGGTGATGTCGG | 909 | DGFGDVGALESLRGNADLAYILSM | 910 |
| Caspase9 | TGCTCTTGAGAGTTTGAGGGGAA | | EPCGHCLIINNVNFCRESGLRTRT | |
| | ATGCAGATTTGGCTTACATCCTGA | | GSNIDCEKLRRRFSSLHFMVEVKG | |
| | GCATGGAGCCCTGTGGCCACTGC | | DLTAKKMVLALLELARQDHGALDC | |
| | CTCATTATCAACAATGTGAACTTC | | CVVVILSHGCQASHLQFPGAVYGT | |
| | TGCCGTGAGTCCGGGCTCCGCAC | | DGCPVSVEKIVNIFNGTSCPSLGG | |
| | CCGCACTGGCTCCAACATCGACT | | KPKLFFIQACGGEQKDHGFE VAST | |
| | GTGAGAAGTTGCGGCGTCGCTTC | | SPEDESPGSNPEPDATPFQEGLR | |
| | TCCTCGCTGCATTTCATGGTGGA | | TFDQLDAISSLPTPSDIFVSYSTFP | |
| | GGTGAAGGGCGACCTGACTGCCA | | GFVSWRDPKSGSWYVETLDDIFE | |
| | AGAAAATGGTGCTGGCTTTGCTG | | QWAHSEDLQSLLLRVANAVSVKGI | |
| | GAGCTGGCGCgGCAGGACCACG | | YKQMPGCFNFLRKKLFFKTSASRA | |
| | GTGCTCTGGACTGCTGCGTGGTG | | | |
| | GTCATTCTCTCTCACGGCTGTCAG | | | |
| | GCCAGCCACCTGCAGTTCCCAGG | | | |
| | GGCTGTCTACGGCACAGATGGAT | | | |
| | GCCCTGTGTCGGTCGAGAAGATT | | | |
| | GTGAACATCTTCAATGGGACCAG | | | |
| | CTGCCCCAGCCTGGGAGGGAAG | | | |
| | CCCAAGCTCTTTTTCATCCAGGCC | | | |
| | TGTGGTGGGGAGCAGAAAGACCA | | | |
| | TGGGTTTGAGGTGGCCTCCACTT | | | |
| | CCCCTGAAGACGAGTCCCCTGGC | | | |
| | AGTAACCCCGAGCCAGATGCCAC | | | |
| | CCCGTTCCAGGAAGGTTTGAGGA | | | |
| | CCTTCGACCAGCTGGACGCCATA | | | |
| | TCTAGTTTGCCCACACCCAGTGA | | | |
| | CATCTTTGTGTCCTACTCTACTTT | | | |
| | CCCAGGTTTTGTTTCCTGGAGGG | | | |
| | ACCCCAAGAGTGGCTCCTGGTAC | | | |
| | GTTGAGACCCTGGACGACATCTT | | | |
| | TGAGCAGTGGGCTCACTCTGAAG | | | |
| | ACCTGCAGTCCCTCCTGCTTAGG | | | |
| | GTCGCTAATGCTGTTTCGGTGAAA | | | |
| | GGGATTTATAAACAGATGCCTGGT | | | |
| | TGCTTTAATTTCCTCCGGAAAAAA | | | |
| | CTTTTCTTTAAAACATCAGCTAGC | | | |
| | AGAGCC | | | |
| T2A | GAGGGCAGGGGAAGTCTTCTAAC | 911 | EGRGSLLTCGDVEENPGP | 912 |
| | ATGCGGGGACGTGGAGGAAAATC | | | |
| | CCGGGCCCtga | | | |

pBP0044-pSH1-FKBP_{V36}.dCaspase9.T2A (from FIG. 42)

| | | SEQ | | SEQ |
|---|---|---|---|---|
| Fragment | Nucleotide | ID NO: | Peptide | ID NO: |
| Linker | ATGCTCGAG | 913 | MLE | 914 |
| FKBP_{V36} | GGAGTGCAGGTGGAgACtATCTCC | 915 | GVQVETISPGDGRTFPKRGQTCV | 916 |
| | CCAGGAGACGGGCGCACCTTCCC | | VHYTGMLEDGKKVDSSRDRNKPF | |
| | CAAGCGCGGCCAGACCTGCGTG | | KFMLGKQEVIRGWEEGVAQMSVG | |
| | GTGCACTACACCGGGATGCTTGA | | QRAKLTISPDYAYGATGHPGIIPPH | |
| | AGATGGAAAGAAAGTTGATTCCTC | | ATLVFDVELLKL | |
| | CCGGGACAGAAACAAGCCCTTTA | | | |
| | AGTTTATGCTAGGCAAGCAGGAG | | | |
| | GTGATCCGAGGCTGGGAAGAAGG | | | |
| | GGTTGCCCAGATGAGTGTGGGTC | | | |
| | AGAGAGCCAAACTGACTATATCTC | | | |
| | CAGATTATGCCTATGGTGCCACT | | | |
| | GGGCACCCAGGCATCATCCCACC | | | |
| | ACATGCCACTCTCGTCTTCGATGT | | | |
| | GGAGCTTCTAAAACTGGAA | | | |
| Linker | TCAGGCGGTGGCTCAGGTGTCGA | 917 | SGGGSGVD | 918 |
| | G | | | |
| Δ- | GTCGACGGATTTGGTGATGTCGG | 919 | DGFGDVGALESLRGNADLAYILSM | 920 |
| Caspase9 | TGCTCTTGAGAGTTTGAGGGGAA | | EPCGHCLIINNVNFCRESGLRTRT | |
| | ATGCAGATTTGGCTTACATCCTGA | | GSNIDCEKLRRRFSSLHFMVEVKG | |
| | GCATGGAGCCCTGTGGCCACTGC | | DLTAKKMVLALLELARQDHGALDC | |
| | CTCATTATCAACAATGTGAACTTC | | CVVVILSHGCQASHLQFPGAVYGT | |
| | TGCCGTGAGTCCGGGCTCCGCAC | | DGCPVSVEKIVNIFNGTSCPSLGG | |
| | CCGCACTGGCTCCAACATCGACT | | KPKLFFIQACGGEQKDHGFEVAST | |
| | GTGAGAAGTTGCGGCGTCGCTTC | | SPEDESPGSNPEPDATPFQEGLR | |
| | TCCTCGCTGCATTTCATGGTGGA | | TFDQLDAISSLPTPSDIFVSYSTFP | |
| | GGTGAAGGGCGACCTGACTGCCA | | GFVSWRDPKSGSWYVETLDDIFE | |
| | AGAAAATGGTGCTGGCTTTGCTG | | QWAHSEDLQSLLLRVANAVSVKGI | |
| | GAGCTGGCGCgGCAGGACCACG | | YKQMPGCFNFLRKKLFFKTSASRA | |
| | GTGCTCTGGACTGCTGCGTGGTG | | | |
| | GTCATTCTCTCTCACGGCTGTCAG | | | |
| | GCCAGCCACCTGCAGTTCCCAGG | | | |
| | GGCTGTCTACGGCACAGATGGAT | | | |
| | GCCCTGTGTCGGTCGAGAAGATT | | | |
| | GTGAACATCTTCAATGGGACCAG | | | |
| | CTGCCCCAGCCTGGGAGGGAAG | | | |
| | CCCAAGCTCTTTTTCATCCAGGCC | | | |
| | TGTGGTGGGGAGCAGAAAGACCA | | | |
| | TGGGTTTGAGGTGGCCTCCACTT | | | |
| | CCCCTGAAGACGAGTCCCCTGGC | | | |
| | AGTAACCCCGAGCCAGATGCCAC | | | |
| | CCCGTTCCAGGAAGGTTTGAGGA | | | |
| | CCTTCGACCAGCTGGACGCCATA | | | |
| | TCTAGTTTGCCCACACCCAGTGA | | | |
| | CATCTTTGTGTCCTACTCTACTTT | | | |
| | CCCAGGTTTTGTTTCCTGGAGGG | | | |
| | ACCCCAAGAGTGGCTCCTGGTAC | | | |
| | GTTGAGACCCTGGACGACATCTT | | | |
| | TGAGCAGTGGGCTCACTCTGAAG | | | |
| | ACCTGCAGTCCCTCCTGCTTAGG | | | |
| | GTCGCTAATGCTGTTTCGGTGAAA | | | |
| | GGGATTTATAAACAGATGCCTGGT | | | |
| | TGCTTTAATTTCCTCCGGAAAAAA | | | |
| | CTTTTCTTTAAAACATCAGCTAGC | | | |
| | AGAGCC | | | |
| T2A | GAGGGCAGGGGAAGTCTTCTAAC | 921 | EGRGSLLTCGDVEENPGP | 922 |
| | ATGCGGGGACGTGGAGGAAAATC | | | |
| | CCGGGCCCtga | | | |

### Example 25: Dual Control of Modified Cells

Chemical Induction of protein Dimerization (CID) has been effectively applied to make cellular suicide or apoptosis inducible with the small molecule homodimerizing ligand, rimiducid (AP1903). This technology underlies the “safety switch” incorporated as a gene therapy adjunct in cell transplants (1, 2). Using this technology, normal cellular regulatory pathways that rely on protein-protein interaction as part of a signaling pathway can be adapted to ligand-dependent, conditional control if a small molecule dimerizing drug is used to control the protein-protein oligomerization event (3-5). Induced dimerization of a fusion protein comprising Caspase-9 and FKBP12 or an FKBP12 variant (i.e., “iCaspase9/iCasp9/iC9) using a homodimerizing ligand, such as rimiducid (AP1903), AP1510 or AP20187, can rapidly effect cell death. (Amara J F (97) PNAS 94:10618-23). Caspase-9 is an initiating caspase that acts as a “gate-keeper” of the apoptotic process (6). Pro-apoptotic molecules (e.g., cytochrome c) released from the mitochondria of apoptotic cells alter the conformation of Apaf-1, a caspase-9-binding scaffold, leading to its oligomerization and formation of the “apoptosome”. This alteration facilitates caspase-9 dimerization and cleavage of its latent form into an active molecule that, in turn, cleaves the “downstream” apoptosis effector, caspase-3, leading to irreversible cell death. Rimiducid binds directly with two FKBP12-V36 moieties and can direct the dimerization of fusion proteins that include FKBP12-V36 (1, 2). iC9 engagement with rimiducid circumvents the need for Apaf1 conversion to the active apoptosome. In this example, the fusion of caspase-9 to protein moieties that engage a heterodimerizing ligand was assayed for its ability to direct its activation and cell death with similar efficacy to rimiducid-mediated iC9 activation.

MyD88 and CD40 were chosen as the basis of the iMC activation switch. MyD88 plays a central signaling role in the detection of pathogens or cell injury by antigen-presenting cells (APCs), like dendritic cells (DCs). Following exposure to pathogen- or necrotic cells-derived “danger” molecules”, a subclass of “pattern recognition receptors”, called Toll-Like Receptors (TLRs) are activated, leading to the aggregation and activation of adapter molecule, MyD88, via homologous TLR-IL1RA (TIR) domains on both proteins. MyD88, in turn, activates downstream signaling, via the rest of the protein. This leads to the upregulation of costimulatory proteins, like CD40, and other proteins, like MHC and proteases, needed for antigen processing and presentation. The fusion of signaling domains from MyD88 and CD40 with two Fv domains, provides iMC (also MCFvvMC.FvFv), which potently activated DCs following exposure to rimiducid (7). It was later found that iMC is a potent costimulatory protein for T cells, as well.

Rapamycin is a natural product macrolide that binds with high affinity (<1 nM) to FKBP12 and together initiates the high-affinity, inhibitory interaction with the FKBP-Rapamycin-Binding (FRB) domain of mTOR (8). FRB is small (89 amino acids) and can thereby be used as a protein “tag” or “handle” when appended to many proteins (9-11). Coexpression of a FRB-fused protein with a second FKBP12-fused protein renders their approximation rapamycin-inducible (12-16). This and the following examples provide experiments and results designed to test whether coexpression of FRB-bound Caspase-9 (iRC9) with FKBP-bound Caspase-9 (iC9) can also direct apoptosis and serve as the basis for a cell safety switch regulated by the orally available ligand, rapamycin, or derivatives of rapamycin (rapalogs) that do not inhibit mTOR at a low, therapeutic dose but instead bind with selected, Caspase-9-fused mutant FRB domains.

Also provided in these examples is another embodiment of the dual-switch technology, (FwtFRBC9/MCFvFv) where a homodimerizer, such as AP1903 (rimiducid), induces activation of a modified cell, and a heterodimerizer, such as rapamycin or a rapalog, activates a safety switch, causing apoptosis of the modified cell. In this embodiment, for example, a chimeric pro-apoptotic polypeptide, such as, for example, Caspase-9, comprising both an FKBP12 and an FRB, or FRB variant region (FwtFRBC9) is expressed in a cell along with an inducible chimeric MyD88/CD40 costimulating polypeptide, that comprises MyD88 and CD40 polypeptides and at least two copies of FKBP12v36 (MC.FvFv). Upon contacting the cell with a dimerizer that binds to the Fv regions, the MC.FvFv dimerizes or multimerizes, and activates the cell. The cell may, for example, be a T cell that expresses a chimeric antigen receptor directed against a target antigen (CAR). As a safety switch, the cell may be contacted with a heterodimerizer, such as, for example, rapamycin, or a rapalog, that binds to the FRB region on the FwtFRB.C9 polypeptide, as well as the FKBP12 region on the FwtFRB.C9 polypeptide, causing direct dimerization of the Caspase-9 polypeptide, and inducing apoptosis. (FIG. 43 (2), FIG. 57). In another mechanism, the heterodimerizer binds to the FRB region on the FwtFRBC9 polypeptide, and the Fv region on the MC.FvFv polypeptide, causing scaffold-induced dimerization, due to the scaffold of two FKBP12v36 polypeptides on each MC.FvFv polypeptide (FIG. 43 (1)), and inducing apoptosis. Nucleic acid constructs that contain both MC.FvFv and FwtFRBC9 have been named FwtFRBC9/MC.FvFv, for purposes of these examples.

In another embodiment of the dual-switch technology, (FRBFwtMC/FvC9) a heterodimerizer, such as rapamycin or a rapalog, induces activation of a modified cell, and a homodimerizer, such as AP1903 activates a safety switch, causing apoptosis of the modified cell. In this embodiment, for example, a chimeric pro-apoptotic polypeptide, such as, for example, Caspase-9, comprising an Fv region (iFvC9) was expressed in a cell along with an inducible chimeric MyD88/CD40 costimulating polypeptide, that comprises MyD88 and CD40 polypeptides and both an FKBP12 and an FRB or FRB variant region (FwtFRBMC) (MC.FvFv). Upon contacting the cell with rapamycin or a rapalog that heterodimerizes the FKBP12 and FRB regions, the FwtFRBMC dimerizes or multimerizes, and activates the cell. The cell may, for example, be a T cell that expresses a chimeric antigen receptor directed against a target antigen (CARζ). As a safety switch, the cell may be contacted with a homodimerizer, such as, for example, AP1903, that binds to the iFvC9 polypeptide, causing direct dimerization of the Caspase-9 polypeptide, and inducing apoptosis. (FIG. 57 (right)). Nucleic acid constructs that contain both iFvC9 and FwtFRBMC have been named FwtFRBMC/FvC9 for purposes of these examples.xxx

### Materials and Methods

### Production of Retroviruses and Transduction of Peripheral Blood Mononuclear Cells (PBMCs)

HEK 293T cells (1.5×10⁵) were seeded on a 100-mm tissue culture dish in 10 mL DMEM4500, supplemented with glutamine, penicillin/streptomycin and 10% fetal calf serum. After 16-30 hours incubation, cells were transfected using Novagen's GeneJuice® protocol. Briefly, for each transfection, 0.5 mL OptiMEM (LifeTechnologies) was pipetted into a 1.5-mL microcentrifuge tube and 30 μL GeneJuice reagent added followed by 5 sec. vortexing. Samples were rested 5 minutes to settle the GeneJuice suspension. DNA (15 μg total) was added to each tube and mixed by pipetting up and down four times. Samples were allowed to rest for 5 minutes for GeneJuice-DNA complex formation and the suspension added dropwise to one dish of 293T cells. A typical transfection included these plasmids to produce replication incompetent retrovirus: 3.75 μg plasmid containing gag-pol (pEQ-PAM3(-E)), 2.5 μg plasmid containing viral envelope (e.g., RD114), Retrovirus containing gene of interest=3=3.75 μg.

PBMCs were stimulated with anti-CD3 and anti-CD28 antibodies precoated to wells of tissue culture plates. 24 hours after plating, 100 U/ml IL-2 was added to the culture. On day 2 or three supernatant containing retrovirus from transfected 293T cells was filtered at 0.45 μm and centrifuged on non-TC treated plates precoated with Retronectin (10 μl per well in 1 ml of PBS per 1 cm² of surface). Plates were centrifuged at 2000 g for 2 hours at room temperature. CD3/CD28 blasts were resuspended at 2.5×10⁵ cells/ml in complete media, supplemented with 100 U/ml IL-2 and centrifuged on the plate at 1000×g for 10 minutes at room temperature. After 3-4 days incubation cells were counted and transduction efficiency measured by flow cytometry using the appropriate marker antibodies (typically CD34 or CD19). Cells were maintained in complete media supplemented with 100 U/ml IL-2, refed cells every 2-3 days with fresh media and IL-2 and split as needed to expand the cells.

### T Cell Caspase Assay in Cultured Cells

After transduction with the appropriate retrovirus, 50,000 T were seeded per well of 96-well plates in the presence or absence of suicide drugs (rimiducid or rapamycin) in CTL medium without IL-2. To enable detection of apoptosis using the IncuCyte instrument, 2 μM of IncuCyte™ Kinetic Caspase-3/7 Apoptosis reagent (Essen Bioscience, 4440) were add to each well to reach a total volume of 200 μl. The plates were centrifuged for 5 min at 400×g and placed inside the IncuCyte (Dual Color Model 4459) to monitor green fluorescence every 2-3 hours for a total of 48 hours at 10× objective. Image analysis was performed using the “Tcells_caspreagent_phase_green_10×_MLD” processing definition. The “Total Green Object Integrated Intensity” metric is used to quantify caspase activation. Each condition was performed in duplicates and each well was imaged at 4 different locations.

### T Cell Anti-Tumor Assay

The HPAC PSCA⁺ tumor cells were stably labeled with nuclear-localized RFP protein using the NucLight™ Red Lentivirus Reagent (Essen Bioscience, 4625). To set up the coculture, 4000 HPAC-RFP cells were seeded per well of 96-well plates in 100 μl of CTL medium without IL-2 for at least 4 hours to allow tumor cells to adhere. After transduction with the appropriate retrovirus and allowed to rest for at least 7 days in culture, T were seeded according to various E:T ratios to the HPAC-RFP-containing 96-well plates. Rimiducid was also added to the culture to reach 300 μl total volume per well. Each plate was set up in duplicates, one plate to monitor with the IncuCyte cell imaging system and one plate for supernatant collection for ELISA assay on day 2. The plates were centrifuged for 5 min at 400×g and placed inside the IncuCyte (Essen Bioscience, Dual Color Model 4459) to monitor red fluorescence (and green fluorescence if T cells were labeled with GFP-Ffluc) every 2-3 hours for a total of 7 days at 10× objective. Image analysis was performed using the “HPAC-RFP_TcellsGFP_10×_MLD” processing definition. On day 7, HPAC-RFP cells were analyzed using the “Red Object Count (1/well)” metric. Also on day 7, 0 or 10 nM of suicide drug were added to each well of the coculture and placed back in the IncuCyte to monitor T cell elimination. On day 8, Tcell-GFP cells were analyzed using the “Total Green Object Integrated Intensity” metric. Each condition was performed at least in duplicates and each well was imaged at 4 different locations.

To measure Raji cell anti-tumor activity populations of cells were determined by flow cytometry rather than incucyte as the cells do not adhere to a plate. Raji cells (ATCC) labeled by stable expression of Green Fluorescent Protein (Raji-GFP) are a Burkitt's lymphoma cell line that express CD19 on the cell surface and are a target for an anti-CD19 CAR. 50000 Raji-GFP cells were seeded on a 24 well plate with 10000 CAR-T cells, a 1:5 E:T ratio. Media supernatant was taken at 48 hours for determination of cytokine release by activated CAR-T cells. The degree of tumor killing was determined at 7 days and 14 days by flow cytometry (Galeos, Beckman-Coulter) by the proportion of GFP labeled tumor cells and CD3 labeled T cells.

### IVIS Imaging

NSG mice with labeled T cells anesthetized with isofluorane and injected with 100 μl D-luciferin (15 mg/ml stock solution in PBS) by an intraperitoneal (i.p.) route in the lower abdomen. After 10 minutes the animals were transferred from the anesthesia chamber to the IVIS platform. Images were acquired from the dorsal and ventral sides with an IVIS imager (Perkin-Elmer), and BLI quantitated and documented with Living Image software (IVIS Imaging Systems).

### Western Blot

After transduction with the appropriate retrovirus, 6,000,000 T cells were seeded per well of 6-well plates in 3 ml CTL medium. Twenty-four hours later, cells were collected, washed in cold PBS, and lysed in RIPA Lysis and Extraction Buffer (Thermo, 89901) containing 1× Halt Protease Inhibitor Cocktail (Thermo, 87786) on ice for 30 min. in the plated. The lysates were centrifuged at 16,000×g for 20 min at 4° C. and the supernatants were transferred to new Eppendorf tubes. Protein assay was performed using the Pierce BCA Protein Assay Kit (Thermo, 23227) per manufacturer's recommendation. To prepare samples for SDS-PAGE, 50 ug of lysates were mixed with 4× Laemmli Sample Buffer (Bio Rad, 1610747) and heat at 95° C. for 10 min. Meanwhile, 10% SDS gels were prepared using Bio Rad casting apparatus and 30% Acrylamide/bis Solution (Bio Rad, 160158). The samples were loaded along with Precision Plus Protein Dual Color Standards (Bio Rad, 1610374) and ran in 1× Tris/glycine Running Buffer (Bio Rad, 1610771) at 140 V for 90 min. After protein separation, the gels were transferred onto PVDF membranes using the program 0 (7 min total) in the iBlot 2 device (Thermo, IB21001). The membranes were probed with primary and secondary antibodies using the iBind Flex Western Device (Thermo, SLF2000) according to manufacturer's recommendation. Anti-MyD88 antibody (Sigma, SAB1406154) was used at 1:200 dilution and the secondary HRP-conjugated goat anti-mouse IgG antibody (Thermo, A16072) was used at 1:500 dilution. The caspase-9 antibody (Thermo, PA1-12506) was used at 1:200 dilution and the secondary HRP-conjugated goat anti-rabbit IgG antibody (Thermo, A16104) was used at 1:500 dilution. The β-actin antibody (Thermo, PA1-16889) was used at 1:1000 dilution and the secondary HRP-conjugated goat anti-rabbit IgG antibody (Thermo, A16104) was used at 1:1000 dilution. The membranes were developed using the SuperSignal West Femto Maximum Sensitivity Substrate Kit (Thermo, 34096) and imaged using the Gel Logic 6000 Pro camera and the CareStream MI software (v.5.3.1.16369).

### Transfection of Cells for Reporter Assay

HEK 293T cells (1.5×10⁵) were seeded on a 100-mm tissue culture dish in 10 mL DMEM4500, supplemented with glutamine, penicillin/streptomycin and 10% fetal calf serum. After 16-30 hours incubation, cells were transfected using Novagen's GeneJuice® protocol. Briefly, for each transfection, 0.5 mL OptiMEM was pipetted into a 1.5-mL microcentrifuge tube and 15 μL GeneJuice reagent added followed by 5 sec. vortexing. Samples were rested 5 minutes to settle the GeneJuice suspension. DNA (5 μg total) was added to each tube and mixed by pipetting up and down four times. Samples were allowed to rest for 5 minutes for GeneJuice-DNA complex formation and the suspension added dropwise to one dish of 293T cells. A typical transfection contains 1 μg NFkB-SEAP (5), 4 μg iMC+CARζ(pBP0774) or 4 μg MC-Rap-CAR (pBP1440) (1).

Stimulation of Cells with Dimerizing Drugs

24 hours following transfection (4.1), 293T cells were split to 96-well plates and incubated with dilutions of dimerizing drugs. Briefly, 100 μL media was added to each well of a 96-well flat-bottom plate. Drugs were diluted in tubes to a concentration 4× the top concentration in the gradient to be place on the plate. 100 μL of dimerizing ligand (rimiducid, rapamycin, isopropoxylrapamycin) was added to each of three wells on the far right of the plate (assays are thereby performed in triplicate). 100 μL from each drug-containing well was then transferred to the adjacent well and the cycle repeated 10 times to produce a serial two-fold step gradient. The last wells were untreated and serve as a control for basal reporter activity. Transfected 293 cells were then trypsinized, washed with complete media, suspended in media and 100 μL aliquoted to each well containing drug (or no drug). Cells were incubated 24 hours.

### Assay of Reporter Activity

The SRα promoter is a hybrid transcriptional element comprising the SV40 early region (which drives T antigen transcription) and parts (R and U5) of the Long Terminal Repeat (LTR) of Human T Cell Lymphotropic Virus (HTLV-1). This promoter drives high, constitutive levels of the Secreted Alkaline Phosphate (SeAP) reporter gene. Activation of caspase-9 by dimerization rapidly leads to cell death and the proportion of cells dying increases with increasing drug amounts. When cells die, transcription and translation of reporter stops but already secreted reporter proteins persists in the media. Loss of constitutive SeAP activity is thereby an effective proxy for drug-dependent activation of cell death.

24 hours after drug stimulation, 96-well plates were wrapped to prevent evaporation and incubated at 65° C. for 2 hours to inactivate endogenous and serum phosphatases while the heat-stable SeAP reporter remains (3, 12, 14). 100 μL samples from each well were loaded into individual wells of a 96-well assay plate with black sides. Samples were incubated with 0.5 mM 4-methylumbelliferyl phosphate (4-MUP) in 0.5 M diethanolamine at pH 10.0 for 4 to 16 hours. Phosphatase activity was measured by fluorescence with excitation at 355 nm and emission at 460 nm. Data was transferred to a Microsoft Excel spreadsheet for tabulation and graphed with GraphPad Prism.

### Production of Isopropyloxyrapamycin

The method of Luengo et al. ((J. Org. Chem 59:6512, (1994)), (17, 18)) was employed. Briefly, 20 mg of rapamycin was dissolved in in 3 mL isopropanol and 22.1 mg of p-toluene sulfonic acid was added and incubated at room temperature with stirring for 4-12 hours. At completion, 5 mL ethyl acetate was added and products were extracted five times with saturated sodium bicarbonate and 3 times with brine (saturated sodium chloride). The organic phase was dried and redissolved in ethyl acetate: hexane (3:1). Stereoisomers and minor products were resolved by FLASH chromatography on a 10 to 15-mL silica gel column with 3:1 ethyl acetate: hexane under 3-4 KPa pressure and fractions dried. Fractions were assayed by spectrophotometry at 237 nM, 267 nM, 278 nM and 290 nM and tested for binding specificity in a FRB allele-specific transcriptional switch.

### Expression of Components of the Activation Switch Technology

Retroviral constructs were created to express fusion proteins between FKBP12 with and without FRB and the inducible target protein. The constructs co-express Chimeric Antigen Receptors (CAR) as part of a gene therapy strategy to direct tumor specific immunity. Inducible (MC.FvFv) or constitutive (MC) costimulatory molecules were also present with the Caspase-9 safety switch. Each component was separated with a 2A cotranslational cleavage site derived from picornaviruses. To better understand how these molecules will function together in target T cells, it was important to determine steady state protein levels in T cells. To determine relative protein expression levels of all components of the “iMC+CARζ-T” (pBP0608; MC.FvFv+CARζ), “i9+CARζ+MC” (pBP0844; iFvCasp9+CARζ+constitutively active “MC”), and (pBP1300; FwtFRBC9/MC.FvFv+CARζ+iMC) vectors, Western blot analysis was performed on transduced T cells from four different donors using antibodies specific for MyD88, caspase-9 and α-actin (FIG. 44A). The results revealed that iMC+CARζ-T T cells express the MC.FvFv component at similar levels to i9+CARζ+MC T cells expressing MC (without fused FKBP12). However, the level of MC.FvFv expression in FwtFRBC9/MC.FvFv T cells was significantly lower than in the other two CAR modified T cells. Similarly, the iFvC9 component in the i9+CARζ+MC construct was expressed at much higher levels compared to the iFwtFRBC9 component (FKBP.FRB.ΔC9) in the FwtFRBC9/MC.FvFv construct, suggesting that the larger multi-cistronic insert was limiting protein expression or that high basal signaling activity from MC was eliminating cells expressing high levels of these chimeric proteins. To distinguish between these possibilities, the stability of CAR expression and basal toxicity in T cells over prolonged culture in vitro was assessed. CAR expression was analyzed by flow cytometry using antibody, QBend-10 (Biolegend), specific for an epitope derived from human CD34 incorporated into the extracellular portion of a 1^{st} generation CAR-ζ, and T cell viability was assessed using a Nexelon Cellometer with the cells stained with acridine orange and propidium iodide cells. Expression analysis by flow cytometry (Galleos, Beckman) demonstrated that iMC+CARζ-T cells express much higher CAR levels compared to i9+CARζ+MC and T cells (FIG. 44B). However, there was relatively no difference in the viability of cells grown in culture between the cells that had been modified with all three CAR T cell types (FIG. 44C). Thus, the difference in chimeric protein expression may have been based on the limiting packaging ability of the viral vector used.

Induction of Apoptosis with FwtFRBC9/MC.FvFv Constructs

To determine whether the FwtFRBC9/MC.FvFv construct was functional despite somewhat lower protein expression per cell, the functionality of the on and off switches incorporated into the FwtFRBC9/MC.FvFv construct design was examined in the absence of target tumor cells. The off switch (iFwtFRBC9), which was activated by rapamycin-induced dimerization of FKBP.FRB.ΔC9, was tested by subjecting T cells from 4 different donors, which were transduced with the iMC+CARζ-T, i9+CARζ+MC, and FwtFRBC9/MC.FvFv vectors, to a caspase-based killing assay using the “Caspase 3/7 Green” reagent (FIG. 45A). In this assay a peptide sensitive to Caspase 3 or 7 was linked with a latent fluorescent DNA intercalating dye. Activation of caspase 3/7 during apoptosis releases the dye permitting DNA binding and green cell fluorescence. A 96-well microplate containing cells was placed inside an IncuCyte machine to monitor activated caspase activity (cleaved caspase 3/7 reagent=green fluorescence) for 48 hours. The IncuCyte is an automated microscope that can observe, quantitate and document live cells cultured on plates with or without fluorescent labels over extended time periods. In the absence of drug, FwtFRBC9/MC.FvFv T cells displayed the highest level of basal toxicity followed by iMC+CARζ-T and i9+CARζ+MC-T cells, respectively. Rimiducid induced activation of iC9 (in i9+CARζ+MC) at a similar efficiency as rapamycin-inducing iFwtFRBC9 at all ligand concentrations (0.8, 4, 20 nM). However, the kinetics of iC9 activation appears to be slightly faster than that of iFwtFRBC9 activation. After 48 hours of suicide drug treatment, cells were analyzed by flow cytometry for the following markers: CD34 (engineered CAR T cell), propidium iodide (PI), Annexin V, and cleaved caspase 3/7 (green fluorescence) (FIG. 45B). A much higher percentage of dead (PI⁺/AnnV⁺) cells was observed in (FwtFRBC9/MC.FvFv) modified T cells (60%) than in i9+CARζ+MC-T cells (20%) 48 hours post-drug treatment, consistent with the high caspase activation level independently observed at later time points in (FwtFRBC9/MC.FvFv) modified T cells using an IncuCyte-based caspase assay. To examine the on-switch, which was activated by rimiducid-induced dimerization of MC.FKBPᵥ.FKBPᵥ (MCFvFv), iMC+CARζ-T and (FwtFRBC9/MC.FvFv) T cells were treated with various rimiducid concentrations, and IL-2 and IL-6 cytokine release was analyzed by ELISA (FIG. 45C). While iMC+CARζ-T cells showed inducible IL-2 and IL-6 production with increasing rimiducid concentration, cytokine production by (FwtFRBC9/MC.FvFv) T cells was relatively weaker. Basal, ligand-independent IL-6 production by i9+CARζ+MC (with MC) was present at a similar level to that of rimiducid-stimulated iMC+CARζT cells. i9+CARζ+MC

High basal caspase activity could present a manufacturing challenge during viral or T cell production. Therefore, the ability of caspase-9 inhibitor, Q-LEHD-OPh (SEQ ID NO: 2364), to counteract basal caspase activity was assayed. Activated iC9 and iRC9 (FwtFRBC9) can be efficiently inhibited with Q-LEHD-OPh (SEQ ID NO: 2364), which did not appear to be toxic to the T cells at levels as high as 100 μM (FIG. 46). Furthermore, as low as 4 μM Q-LEHD-OPh (SEQ ID NO: 2364) was able to efficiently inhibit caspase-9 activation by iC9 and iRC9 (FwtFRBC9) when they were incubated with 20 nM of the respective activating ligands (FIG. 46C).

Another approach to attenuate high basal caspase activity is to utilize the FRB-T2098L (“FRB_{L}”) mutant that destabilizes protein expression in the iRC9 (FwtFRBC9) construct (15, 16). Additionally, a caspase-9 mutant (N405Q, ΔCasp9_{Q}) also reduces basal caspase activity in iC9. When investigated using the IncuCyte and caspase 3/7 green reagent, both FRB_{L} and Δcasp9_{Q} mutant iRC9 (FwtFRBC9) exhibited lower basal caspase activity compared to wild-type iRC9 (FwtFRBC9) (FIG. 47A). However, changing FRB from the wild-type (Threonine 2098) to the FRB_{L} mutant (Leucine 2098) reduced the maximum killing efficiency by iRC9 (FwtFRBC9) by approximately 50%. Similarly, changing Δcaspase-9 from wt to the N405Q mutant diminished iRC9 (FwtFRBC9) activity to even lower levels than the FRB_{L} mutation.

### Efficiency of Apoptosis Induction by Dimerizer Mediated Binding or Indirect Recruitment to a Scaffold

In this example, an inducible Caspase-9 polypeptide, comprising an FRB region (iFRBC9) was tested in modified cells that also expressed MC.FvFv. Here, in iRC9, rapamycin-induced dimerization of FRB.ΔC9 relies solely on the FKBP-based scaffold provided by the tandem FKBP12 proteins in MC.FKBP_{V}.FKBP_{V}(iMC) co-expressed within the same construct (see FIG. 48A for schematic). In this strategy, recruitment of multiple iFRBC9 molecules to the scaffold of FKBPs (e.g., scaffold of FKBP12v36s) facilitates their indirect spontaneous association and activation. To directly compare the extent of caspase activation between iC9 (pBP0844), iRC9 (pBP1116), and iRC9 (pBP1300), activated T cells were transduced with retrovirus encoding iMC+CARζ-T, i9+CARζ+MC, iFRBC9 and MC.FvFv, or (FwtFRBC9/MC.FvFv) and treated with no drug, 20 nM rapamycin or 20 nM rimiducid and cultured in the presence of caspase 3/7 green reagent (FIG. 48B-D). Although there was generally low basal caspase activity in all of the constructs, cells transduced with (FwtFRBC9/MC.FvFv) exhibited the highest basal caspase activity relative to the other CAR T cells (FIG. 48B). When induced with 20 nM rapamycin, (iFRBC9 and MC.FvFv) demonstrated modest caspase activation, while there was robust induction of caspase activity in T cells (FwtFRBC9/MC.FvFv). (FIG. 48C). This induction of apoptosis was similar in T cells expressing i9+CARζ+MC treated with 20 nM rimiducid (FIG. 48D). In this assay, 20 nM rimiducid was unable to induce dimerization of FKBP.FRB.Δcasp9 (iRC9). This is because of the 1000-fold reduction in affinity of rimiducid for wild-type FKBP present in iRC9 (iFwtFRBC9) relative to FKBP_{V36}.

### Whole Animal Model Assays

To demonstrate the functionality of iRC9 (FwtFRBC9) in vivo, NOD-Scid-IL-2Receptor−/− mice (NSG, Jackson Labs) were injected i.v. with 1×10⁷ iMC+CARζ-T, i9+CARζ+MC, iFRBC9 and MC.FvFv or (FwtFRBC9/MC.FvFv) T cells co-transduced with GFP-FFluc per mouse. Bioluminescence imaging (BLI) of CAR T cells was assessed 18 hours (˜18 h) prior to drug treatment, immediately before drug treatment (0 h) and 4.5 h, 18 h, 27 h, and 45 h post-drug treatment (FIGS. 49A & B). A subset of mice that received i9+CARζ+MC T cell injections were treated i.p. with 5 mg/kg rimiducid, while a subset of mice that received iMC+CARζ-T, (iFRBC9 and MC.FvFv) and -2.0 T cells were treated i.p. with 10 mg/kg rapamycin. All other mice received vehicle only i.p. At 45 h post-drug treatment, mice were euthanized, and blood and spleen were collected for flow cytometry analysis with antibodies to human (h) CD3 or CD34, and murine (m) CD45. Similar to iC9, iRC9 (iFwtFRBC9) quickly and efficiently eliminated FwtFRBC9/MC.FvFv T cells as assessed by BLI and analysis of blood and spleen tissues (FIGS. 49C & D). Induction of (iFRBC9 and MC.FvFv) T cell apoptosis was modest with delayed kinetics compared to i9+CARζ+MC and FwtFRBC9/MC.FvFv, consistent with in vitro cell death data presented in FIG. 48.

### FwtFRBC9/MC.FvFv Contains a Dual Costimulatory on Switch and Apoptotic Off Switch

To examine the functionality of both on- and off-switches in the FwtFRBC9/MC.FvFv construct in the presence of target tumor cells, T cells were labeled with GFP-FFluc (expressing a Green Fluorescent Protein fused with firefly luciferase as a cell marker in vivo) and co-transduced with PSCA-iMC+CARζ-T (pBP0189), i9+CARζ+MC (pBP0873), or FwtFRBC9/MC.FvFv (pBP1308)-encoding vectors (FIG. 50). Ten days post-transduction, T cells were seeded into 96-well plates at 1:2 and 1:5 effector to tumor target (E:T) ratios with HPAC pancreatic carcinoma cells constitutively labeled with RFP in the presence of 0, 2, or 10 nM rimiducid and placed in the IncuCyte machine to monitor the kinetics of HPAC-RFP and T cell-GFP growth. Two days post-seeding, culture supernatant was analyzed for IL-2, IL-6, and IFN-γ production by ELISA. Overall, iMC+CARζ-T cells produced approximately 3-fold higher levels of IL-2, IL-6, and IFN-γ compared to FwtFRBC9/MC.FvFv T cells at all rimiducid concentrations and both E:T ratios (FIGS. 50A & B). Additionally, the basal activity of the MC co-stimulatory component in the i9+CARζ+MC construct induced IL-6 and IFN-γ cytokine production at similar levels to that measured in rimiducid-stimulated iMC+CARζ-T cells. As seen in FIGS. 50C & D, less than 5% and 10% HPAC-RFP cells remained at 1:2 and 1:5 ratios, respectively. While (FwtFRBC9/MC.FvFv) T cells demonstrated rimiducid-dependent tumor cell killing at both ratios, iMC+CARζ-T cells appear to be rimiducid-independent at these ratios and of similar target killing efficiency as i9+CARζ+MC T cells. When analyzed for T cell expansion, FwtFRBC9/MC.FvFv.0 T cells proliferated and expanded with increasing rimiducid concentration, while iMC+CARζ-T cells were not able to expand to the same extent following 10 nM rimiducid stimulation. Administration of 10 nM rapamycin on day 7 of co-culture resulted in the elimination of more than 60% of (FwtFRBC9/MC.FvFv) T cells within 24 hours while 10 nM rimiducid caused reduction of approximately 50% of i9+CARζ+MC T cells, suggesting that the safety switch is also functional in FwtFRBC9/MC.FvFv.

### Caspase-9 Activation in FwtFRBC9/MC.FvFv

Activation of iRC9 (iFwtFRBC9) within the FwtFRBC9/MC.FvFv-modified T cells could be mediated by both FKBP.FRB.ΔC9 homo-dimerization and scaffold-mediated recruitment driven by recruitment of FRB in FKBP.FRB.C9 to FKBP in MC.FKBP_{V}.FKBP_{V}. To disrupt the ability of iRC9 (iFwtFRBC9) from being activated by scaffold-mediated recruitment, FwtFRBC9/MC.FvFv-related family vectors were generated containing MC.FKBP_{V}.FKBP_{V} (pBP1308, “iMC”), MC.FKBP_{V} (pBP1319, 1 FKBPv), MC (pBP1320, no FKBPs), and MC.FKBP_{V}.FKBP (pBP1321, 1 FKBPᵥ and 1 non-AP1903-binding wild-type FKBP) (see FIG. 51A for schematic of the constructs). PSCA-i9+CARζ+MC vector (pBP0873) served as a positive control for the off-switch and the CD19-iMC+CARζ-T vectors (pBP0608 with MC.FKBP_{V}.FKBP_{V} & pBP1439 with MC.FKBP_{V}) served as positive controls for the on-switch. Protein expression of the CAR-T cells using an anti-MyD88 antibody was determined. Removing 1 copy of FKBPᵥ from iMC resulted in increased MC fusion-protein expression in the FwtFRBC9/MC.FvFv platform (compare pBP1308 versus pBP1319) and the iMC+CARζ-T platform (compare pBP0608 versus pBP1439) (FIG. 51B). However, MC expression was reduced in the construct that contains MC.FKBP_{V}.FKBP (compare pBP1319 versus pBP1321), suggesting that the additional FKBP domain destabilized the MC-fusion protein. Most interestingly, the expression pattern of the i9+CARζ+MC platform constructs (i.e., pBP0873 containing iC9 and pBP1320 containing iRC9 (iFwtFRBC9)) reveal additional slow migrating bands when probed with anti-MyD88 antibody. In addition to the predicted 27 kDa MC fusion protein band, there are 3 additional bands detected at 90, 80 and 50 kDa. Based on the high basal MC signaling in i9+CARζ+MC vectors, this data may support the hypothesis that there is incomplete protein separation at the 2^{nd} “2A” site, resulting in the following candidate protein products: αPSCA.Q.CD8stm.ζ.2A-MC and CD8stm.ζ.2A-MC with the latter losing the scFv domain. In terms of caspase-9-fusion protein expression, there was no marked difference in chimeric caspase protein levels between the different variations of MC-fusion proteins (compare pBP1308, pBP1319, pBP1320, and pBP1321).

To test the off-switch, T cells transduced with the above vectors were subjected to a caspase activation assay with treatment of 0, 0.8, 4, 20 nM rapamycin. T cells transduced with the i9+CARζ+MC vector (pBP0873) were treated with rimiducid. Caspase activation 24 hours post-rapamycin (or rimiducid) exposure was determined and depicted by line graphs (FIG. 51C). Removing 1 copy of FKBPv from iMC actually resulted in improved caspase activation in the FwtFRBC9/MC.FvFv platform (iFwtFRBC9) (compare pBP1308 versus pBP1319). When both copies of FKBPv were removed, caspase activity resembled that of iC9 in terms of kinetics, but at much higher amplitude (compare pBP0873 versus pBP1320). In the construct that contains MC.FKBP_{V}.FKBP, caspase activity reverted to a level comparable to that in the construct encoding original “iMC” MC.FKBP_{V}.FKBP_{V} (compare pBP1308 versus pBP1321).

Topology of FRB and FKBP in iRC9 (iFwtFRBC9)

Since the order and spacing of signaling elements and binding domains might possibly affect outcomes, the order of ligand-binding domains with the iFwtFRBC9 molecules was tested. The iRC9 (iFwtFRBC9) discussed above contained an amino terminal FKBP followed by a FRB domain, as in FKBP.FRB.ΔC9 (pBP1308 and pBP1311). To investigate the efficacy of the opposite configuration, FRB.FKBP.ΔC9/(pBP1310) was constructed (FIG. 51A). A caspase activation assay revealed that FRB.FKBP.ΔC9 was slightly more sensitive than FKBP.FRB.ΔC9 in terms of rapamycin-initiated apoptosis (FIG. 51D). This modest difference is consistent with the higher FRB.FKBP.ΔC9 protein levels compared to the FKBP.FRB.ΔC9 (FIG. 51B). Furthermore, since these two plasmids do not contain the dilutive iMC-associated scaffold, these data also provide evidence that iRC9 does not require scaffold to potently activate caspase signaling. In terms of the on-switch, all FwtFRBC9/MC.FvFv constructs (pBP1308, pBP1319, and pBP1321) exhibit low IL-2 and IL-6 cytokine production in the absence of tumor even when stimulated with rimiducid, while the rimiducid-inducible iMC+CARζ-T constructs (pBP0608 and pBP1439) demonstrate ligand-dependent activation, as expected (data not shown). Moreover, both of the i9+CARζ+MC constructs, containing MC (pBP0873 and pBP1320), induce high basal IL-6 production.

Since iRC9 contains the wild-type FKBP domain, the concentration of rimiducid capable of triggering dimerization and iRC9 activation was assayed to gauge the therapeutic window of safety for using rimiducid as a T cell stimulatory drug. In this assay, 293 cells were transiently transfected with vectors expressing iC9 and the two similar iRC9 variants (FRB.FKBP.ΔC9 and FKBP.FRB.ΔC9) (FIG. 52) and treated with half-log dilution of either rapamycin or rimiducid. Cells were subjected to either the caspase activation assay in the presence of caspase 3/7 green reagent and monitored by IncuCyte (FIG. 52A) or the secreted alkaline phosphatase (SEAP) assay using the constitutive SRα reporter (FIG. 52B). For FIG. 52B left graph, the lines of the graph, as indicated at the 10³ point of the x-axis are, from top to bottom, negative control, FKBP.FRB.C9, FRB.FKBP.C9, iC9. For FIG. 52B right graph, the lines of the graph at the 10³ point of the x-axis are, from top to bottom negative control, iC9, FKBP.FRB.C9, and FRB.FKBP.C9.

Functionally, iRC9 and iC9 appeared to induce caspase cleavage with similar kinetics and threshold when activated by their respective suicide drugs. iRC9 was highly active even in the presence of as little as 100 μM rapamycin, with some efficacy at even lower drug levels albeit with reduced kinetics. When comparing FRB.FKBP.ΔC9 versus FKBP.FRB.ΔC9, FRB.FKBP.ΔC9 was active at lower rapamycin concentration than FKBP.FRB.ΔC9, consistent with data obtained in FIG. 51D. Furthermore, iRC9 was insensitive to rimiducid below 100 nM, which provides a large window of safety to use rimiducid to induce T cell activation (generally at 1 to 10 nM). This experiment also demonstrates that (iFwtFRBC9) is a potent activator of apoptosis that is independent of scaffolding-induced dimerization provided by MC.FvFv.

### MC-Rap: An Inducible Costimulatory Polypeptide Directed by Rapamycin Analogs

To demonstrate the versatility of utilizing tandem fusion of FKBP and FRB to facilitate homodimerization with rapamycin or rapalogs a MC-Rap (iFRBFwtMC) construct was made, which had a MyD88/CD40 fusion with wild-type FKBP and FRB_{L}. MC-Rap was expressed together with a CAR directed against CD19 with the two cistrons separated by a 2A sequence (FIG. 53). With this construct, a rapalog was chosen to bind to the wild-type FKBP present on MC-Rap and together facilitate dimerization with the FRB present on a second MC-Rap. To determine if dimerization of MC-Rap with this technique could direct activation of MC and costimulatory function, retroviral construct 1440 containing MC-Rap was compared with two iMC+CARζ constructs containing the same CAR but which include two tandem copies of rimiducid sensitive Fv or an uninducible MC only construct (1151). When transduced into T cells, the expression of IL-6 which relies on MC function was observed at moderate levels with MC activity alone and was not induced with either the rapalog C7-isobutyloxyrapamycin or rimiducid (FIG. 54). IL-6 induction from the iMC+CARζ-T cells containing either BP0774 with Fv.Fv fused to the carboxy terminus of MC or BP1433 with amino terminal Fv fusions secreted high levels of IL-6 in the presence of but not with isobutyloxyrapamycin. The term “tethered” in FIG. 54 refers to FRB and FKBP polypeptides tethered to a MyD88-CD40 polypeptide. In contrast, BP1440 which expresses MC with a carboxy terminal fusion of wild-type FKBP in tandem with FRB_{L} was not responsive to rimiducid, but strongly induces IL-6 secretion by activation of MC. When probed with an antibody to MyD88 in a western blot, the expression levels of MC.FK_{WT}.FRB_{L} were similar to those expressed by 1433 (also a carboxyl terminal fusion but with Fᵥs) and MC alone (FIG. 55). The dose responsiveness of the iMC+CARζ and MC-Rap-CAR constructs was determined in a sensitive reporter assay in which signaling through MC activates the transcription factor NF-κB (FIG. 56). BP774 was strongly induced by subnanomolar concentrations of rimiducid but not by rapamycin or isobutyloxyrapamycin. In contrast subnanomolar concentrations of rapamycin or isobutyloxyrapamycin were sufficient to induce MC-Rap in BP1440 but rimiducid even at 50 nM remained inert to MC function because of the specificity of the drug for Fᵥ.

(FRBFwtMC/FvC9): A Dual-Switch Activating Costimulation with Rapalog and Apoptosis with Rimiducid

The specificity of MC-Rap for activation with rapalogs but not with rimiducid permitted its employment as a second dual-switch (FRBFwtMC/FvC9) (FIG. 57). In this strategy MC-Rap was coexpressed with a first generation CAR and iC9. Rimiducid was used to activate caspase-9 as a safety switch while the rapalog isobutyloxyrapamycin which binds with FRB_{L} at concentrations 20 fold lower than the wild-type FRB in mTOR (which would inhibit T cell function) can specifically activate MC-Rap. This scheme was the reverse of (FwtFRBC9/MC.FvFv) which activates apoptosis with rapamycin (or rapalog) and activates costimulation with iMC and rimiducid. The drug specificity of the two strategies was demonstrated in a cell killing assay in culture (FIG. 58). The i9+CARζ+MC construct BP0844 which encodes a CD19CAR with iC9 and a constitutive or BP1160 expressing FRBFwtMC/FvC9 or BP1300 expressing FwtFRBC9/MC.FvFv was cocultured with the Raji Burkitt lymphoma cell line that expresses CD19. Tumor killing was ablated by activation of the safety switch with rimiducid both with the i9+CARζ+MC or FRBFwtMC/FvC9 formats. In contrast rapamycin or isobutyloxyrapamycin activated the iRC9 in FwtFRBC9/MC.FvFv and specifically ablated the immune response to tumor.

### REFERENCES

The following references are referred to in the present Example, and are hereby incorporated by reference herein in the present application, in their entireties.
1. Straathof K C, Pule M A, Yotnda P, Dotti G, Vanin E F, Brenner M K, Heslop H E, Spencer D M, and Rooney C M. An inducible caspase 9 safety switch for T-cell therapy. Blood. 2005; 105(11):4247-54.
2. Fan L, Freeman K W, Khan T, Pham E, and Spencer D M. Improved artificial death switches based on caspases and FADD. Hum Gene Ther. 1999; 10(14):2273-85.
3. Spencer D M, Wandless T J, Schreiber S L, and Crabtree G R. Controlling signal transduction with synthetic ligands. Science. 1993; 262(5136):1019-24.
4. Acevedo V D, Gangula R D, Freeman K W, Li R, Zhang Y, Wang F, Ayala G E, Peterson L E, Ittmann M, and Spencer D M. Inducible FGFR-1 activation leads to irreversible prostate adenocarcinoma and an epithelial-to-mesenchymal transition. Cancer Cell. 2007; 12(6):559-71.
5. Spencer D M, Belshaw P J, Chen L, Ho S N, Randazzo F, Crabtree G R, and Schreiber S L. Functional analysis of Fas signaling in vivo using synthetic inducers of dimerization. Curr Biol. 1996; 6(7):839-47.
6. Strasser A, Cory S, and Adams J M. Deciphering the rules of programmed cell death to improve therapy of cancer and other diseases. EMBO J. 2011; 30(18):3667-83.
7. Narayanan P, Lapteva N, Seethammagari M, Levitt J M, Slawin K M, and Spencer D M. A composite MyD88/CD40 switch synergistically activates mouse and human dendritic cells for enhanced antitumor efficacy. J Clin Invest. 2011; 121(4):1524-34.
8. Sabatini D M, Erdjument-Bromage H, Lui M, Tempst P, and Snyder S H. RAFT1: a mammalian protein that binds to FKBP12 in a rapamycin-dependent fashion and is homologous to yeast TORs. Cell. 1994; 78(1):35-43.
9. Brown E J, Albers M W, Shin T B, Ichikawa K, Keith C T, Lane W S, and Schreiber S L. A mammalian protein targeted by G1-arresting rapamycin-receptor complex. Nature. 1994; 369(6483):756-8.
10. Chen J, Zheng X F, Brown E J, and Schreiber S L. Identification of an 11-kDa FKBP12-rapamycin-binding domain within the 289-kDa FKBP12-rapamycin-associated protein and characterization of a critical serine residue. Proc Natl Acad Sci USA. 1995; 92(11):4947-51.
11. Choi J, Chen J, Schreiber S L, and Clardy J. Structure of the FKBP12-rapamycin complex interacting with the binding domain of human FRAP. Science. 1996; 273(5272):239-42.
12. Ho S N, Biggar S R, Spencer D M, Schreiber S L, and Crabtree G R. Dimeric ligands define a role for transcriptional activation domains in reinitiation. Nature. 1996; 382(6594):822-6.
13. Klemm J D, Beals C R, and Crabtree G R. Rapid targeting of nuclear proteins to the cytoplasm. Curr Biol. 1997; 7(9):638-44.
14. Bayle J H, Grimley J S, Stankunas K, Gestwicki J E, Wandless T J, and Crabtree G R. Rapamycin analogs with differential binding specificity permit orthogonal control of protein activity. Chem Biol. 2006; 13(1):99-107.
15. Stankunas K, Bayle J H, Gestwicki J E, Lin Y M, Wandless T J, and Crabtree G R. Conditional protein alleles using knockin mice and a chemical inducer of dimerization. Mol Cell. 2003; 12(6):1615-24.
16. Stankunas K, Bayle J H, Havranek J J, Wandless T J, Baker D, Crabtree G R, and Gestwicki J E. Rescue of Degradation-Prone Mutants of the FK506-Rapamycin Binding (FRB) Protein with Chemical Ligands. Chembiochem. 2007.
17. Liberles S D, Diver S T, Austin D J, and Schreiber S L. Inducible gene expression and protein translocation using nontoxic ligands identified by a mammalian three-hybrid screen. Proc Natl Acad Sci USA. 1997; 94(15):7825-30.
18. Luengo J I, Yamashita D S, Dunnington D, Beck A K, Rozamus L W, Yen H K, Bossard M J, Levy M A, Hand A, Newman-Tarr T, et al. Structure-activity studies of rapamycin analogs: evidence that the C-7 methoxy group is part of the effector domain and positioned at the FKBP12-FRAP interface. Chem Biol. 1995; 2(7):471-81.

### APPENDICES TO THE PRESENT EXAMPLE

APPENDIX 1
pBP1300—pSFG-FKBP.FRB.ΔC9.T2A-αCD19.Q.CD8stm.ζ.P2A-iMC

| | | SEQ | | SEQ |
|---|---|---|---|---|
| Fragment | Nucleotide | ID NO: | Peptide | ID NO: |
| Leader | ATGCtcgagcaattg | 926 | MLEQL | 927 |
| peptide | | | | |
| FKBP″(wt) | GGcGTGCAaGTGGAaACTATaAGC | 928 | GVQVETISPGDGRTFPKRGQTCV | 929 |
| | CCgGGAGAcGGCcGcACATTtCCC | | VHYTGMLEDGKKFDSSRDRNKPF | |
| | AAgAGAGGcCAGACcTGCGTgGTG | | KFMLGKQEVIRGVVEEGVAQMSVG | |
| | CAcTATACaGGAATGCTGGAgGAC | | QRAKLTISPDYAYGATGHPGIIPPH | |
| | GGgAAGAAaTTCGAtAGCtcCCGGG | | ATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 930 | ggql | 931 |
| FRB | gaaatgTGGCATGAAGGGTTGGAAG | 932 | EMWHEGLEEASRLYFGERNVKG | 933 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 934 | SGGGSGPW | 935 |
| Δcaspase9 | GGATTTGGTGATGTCGGTGCTCT | 936 | GFGDVGALESLRGNADLAYILSME | 937 |
| | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFNFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ggatctggaccgcGG | 938 | GSGPR | 939 |
| T2A | GAAGGCCGAGGGAGCCTGCTGA | 940 | EGRGSLLTCGDVEENPGP | 941 |
| | CATGTGGCGATGTGGAGGAAAAC | | | |
| | CCAGGACCA | | | |
| Linker | CCATGG | 942 | PW | 943 |
| Signal | ATGGAGTTTGGACTTTCTTGGTTG | 944 | MEFGLSWLFLVAILKGVQCSR | 945 |
| Peptide | TTTTTGGTGGCAATTCTGAAGGGT | | | |
| | GTCCAGTGTAGCAGG | | | |
| FMC63 VL | GACATCCAGATGACACAGACTAC | 946 | DIQMTQTTSSLSASLGDRVTISCR | 947 |
| | ATCCTCCCTGTCTGCCTCTCTGG | | ASQDISKYLNVVYQQKPDGTVKLLI | |
| | GAGACAGAGTCACCATCAGTTGC | | YHTSRLHSGVPSRFSGSGSGTDY | |
| | AGGGCAAGTCAGGACATTAGTAA | | SLTISNLEQEDIATYFCQQGNTLPY | |
| | ATATTTAAATTGGTATCAGCAGAA | | TFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 948 | gggsgggg | 949 |
| | GC | | | |
| FMC63 VH | GAGGTGAAACTGCAGGAGTCAGG | 950 | EVKLQESGPGLVAPSQSLSVTCTV | 951 |
| | ACCTGGCCTGGTGGCGCCCTCAC | | SGVSLPDYGVSWIRQPPRKGLEW | |
| | AGAGCCTGTCCGTCACATGCACT | | LGVIWGSETTYYNSALKSRLTIIKD | |
| | GTCTCAGGGGTCTCATTACCCGA | | NSKSQVFLKMNSLQTDDTAIYYCA | |
| | CTATGGTGTAAGCTGGATTCGCC | | KHYYYGGSYAMDYWGQGTSVTV | |
| | AGCCTCCACGAAAGGGTCTGGAG | | SS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 952 | gs | 953 |
| CD34 | GAACTTCCTACTCAGGGGACTTTC | 954 | ELPTQGTFSNVSTNVS | 955 |
| epitope | TCAAACGTTAGCACAAACGTAAGT | | | |
| CD8 stalk | CCCGCCCCAAGACCCCCCACACC | 956 | PAPRPPTPAPTIASQPLSLRPEAC | 957 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 958 | IYIWAPLAGTCGVLLLSLVITLYCNH | 959 |
| trans- | GGCACCTGTGGAGTCCTTCTGCT | | RNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 960 | VD | 961 |
| CD3 ζ | AGAGTGAAGTTCAGCAGGAGCGC | 962 | RVKFSRSADAPAYQQGQNQLYNE | 963 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPPR | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 964 | GTRGSG | 965 |
| P2A | GCTACTAACTTCAGCCTGCTGAA | 966 | ATNFSLLKQAGDVEENPGP | 967 |
| | GCAGGCTGGAGACGTGGAGGAG | | | |
| | AACcccgggcct | | | |
| MyD88 | atggctgcaggaggtcccggcgcggggtctgcg | 968 | SEQ ID NO: 305 | 969 |
| | gccccggtctcctccacatcctcccttcccctggct | | MAAGGPGAGSAAPVSSTSSLPLA | |
| | gctctcaacatgcgagtgcggcgccgcctgtctct | | ALNMRVRRRLSLFLNVRTQVAAD | |
| | gttcttgaacgtgcggacacaggtggcggccga | | WTALAEEMDFEYLEIRQLETQADP | |
| | ctggaccgcgctggcggaggagatggactttga | | TGRLLDAWQGRPGASVGRLLDLL | |
| | gtacttggagatccggcaactggagacacaagc | | TKLGRDDVLLELGPSIEEDCQKYIL | |
| | ggaccccactggcaggctgctggacgcctggc | | KQQQEEAEKPLQVAAVDSSVPRT | |
| | agggacgccctggcgcctctgtaggccgactgc | | AELAGITTLDDPLGHMPERFDAFIC | |
| | tcgatctgcttaccaagctgggccgcgacgacgt | | YCPSDI | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgcta | | | |
| | ttgccccagcgacatc | | | |
| Linker | gtcgag | 970 | VE | 971 |
| CD40 | aaaaaggtggccaagaagccaaccaataagg | 972 | KKVAKKPTNKAPHPKQEPQEINFP | 973 |
| | ccccccaccccaagcaggagccccaggagat | | DDLPGSNTAAPVQETLHGCQPVT | |
| | caattttcccgacgatcttcctggctccaacactgc | | QEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 974 | VE | 975 |
| FKBP_{V}_{^{′}} | GGcGTcCAaGTcGAaACcATtagtCCc | 976 | GVQVETISPGDGRTFPKRGQTCV | 977 |
| | GGcGAtGGcaGaACaTTtCCtAAaaG | | VHYTGMLEDGKKVDSSRDRNKPF | |
| | gGGaCAaACaTGtGTcGTcCAtTAtA | | KFMLGKQEVIRGVVEEGVAQMSVG | |
| | CaGGcATGtTgGAgGAcGGcAAaAA | | QRAKLTISPDYAYGATGHPGIIPPH | |
| | gGTgGAcagtagtaGaGAtcGcAAtAAa | | ATLVFDVELLKLE | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cGGaATtATtCCcCCtCAcGCtACctTg | | | |
| | GTgTTtGAcGTcGAaCTgtTgAAgCTc | | | |
| | GAa | | | |
| Linker | gtcgag | 978 | VE | 979 |
| FKBP_{V} | ggagtgcaggtggagactatctccccaggagac | 980 | GVQVETISPGDGRTFPKRGQTCV | 981 |
| | gggcgcaccttccccaagcgcggccagacctg | | VHYTGMLEDGKKVDSSRDRNKPF | |
| | cgtggtgcactacaccgggatgcttgaagatgg | | KFMLGKQEVIRGVVEEGVAQMSVG | |
| | aaagaaagttgattcctcccgggacagaaaca | | QRAKLTISPDYAYGATGHPGIIPPH | |
| | agccctttaagtttatgctaggcaagcaggaggt | | ATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 982 | stop | |

APPENDIX 2
pBP1308—pSFG-FKBP.FRB.ΔC9.T2A-αPSCA.Q.CD8stm.ζ.P2A-iMC

| | | SEQ | | SEQ |
|---|---|---|---|---|
| Fragment | Nucleotide | ID NO: | Peptide | ID NO: |
| Leader | ATGCtcgagcaattg | 983 | MLEQL | 984 |
| peptide | | | | |
| FKBP″(wt) | | | | |
| | GGcGTGCAaGTGGAaACTATaAGC | 985 | GVQVETISPGDGRTFPKRGQTCV | 986 |
| | CCgGGAGAcGGCcGcACATTtCCC | | VHYTGMLEDGKKFDSSRDRNKPF | |
| | AAgAGAGGcCAGACcTGCGTgGTG | | KFMLGKQEVIRGVVEEGVAQMSVG | |
| | CAcTATACaGGAATGCTGGAgGAC | | QRAKLTISPDYAYGATGHPGIIPPH | |
| | GGgAAGAAaTTCGAtAGCtcCCGGG | | ATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 987 | ggql | 988 |
| FRB | gaaatgTGGCATGAAGGGTTGGAAG | 989 | EMWHEGLEEASRLYFGERNVKG | 990 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 991 | SGGGSGPW | 992 |
| Δcaspase9 | GGATTTGGTGATGTCGGTGCTCT | 993 | GFGDVGALESLRGNADLAYILSME | 994 |
| | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFNFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ggatctggaccgcGG | 995 | GSGPR | 996 |
| T2A | GAAGGCCGAGGGAGCCTGCTGA | 997 | EGRGSLLTCGDVEENPGP | 998 |
| | CATGTGGCGATGTGGAGGAAAAC | | | |
| | CCAGGACCA | | | |
| Linker | CCATGG | 999 | PW | 1000 |
| Signal | ATGGAGTTTGGACTTTCTTGGTTG | 1001 | MEFGLSVVLFLVAILKGVQCSR | 1002 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1003 | DIQLTQSPSTLSASMGDRVTITCSA | 1004 |
| VL | ATCTACACTCAGCGCTAGCATGG | | SSSVRFIHWYQQKPGKAPKRLIYD | |
| | GGGACAGGGTCACAATCACGTGC | | TSKLASGVPSRFSGSGSGTDFTLT | |
| | TCTGCCTCAAGTTCCGTTAGGTTT | | ISSLQPEDFATYYCQQWGSSPFTF | |
| | ATCCATTGGTATCAGCAGAAACCT | | GQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1005 | gggsgggg | 1006 |
| | GC | | | |
| PSCA(A11) | GAGGTGCAGCTCGTGGAGTATGG | 1007 | EVQLVEYGGGLVQPGGSLRLSCA | 1008 |
| VH | CGGGGGCCTGGTGCAGCCTGGG | | ASGFNIKDYYIHVVVRQAPGKGLE | |
| | GGTAGTCTGAGGCTCTCCTGCGC | | WVAWIDPENGDTEFVPKFQGRAT | |
| | TGCCTCTGGCTTTAACATTAAAGA | | MSADTSKNTAYLQMNSLRAEDTA | |
| | CTACTACATACATTGGGTGCGGC | | VYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1009 | gs | 1010 |
| CD34 | GAACTTCCTACTCAGGGGACTTTC | 1011 | ELPTQGTFSNVSTNVS | 1012 |
| epitope | TCAAACGTTAGCACAAACGTAAGT | | | |
| CD8 stalk | CCCGCCCCAAGACCCCCCACACC | 1013 | PAPRPPTPAPTIASQPLSLRPEAC | 1014 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1015 | IYIWAPLAGTCGVLLLSLVITLYCNH | 1016 |
| trans- | GGCACCTGTGGAGTCCTTCTGCT | | RNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1017 | VD | 1018 |
| CD3ζ | AGAGTGAAGTTCAGCAGGAGCGC | 1019 | RVKFSRSADAPAYQQGQNQLYNE | 1020 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPPR | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 1021 | GTRGSG | 1022 |
| P2A | GCTACTAACTTCAGCCTGCTGAA | 1023 | ATNFSLLKQAGDVEENPGP | 1024 |
| | GCAGGCTGGAGACGTGGAGGAG | | | |
| | AACcccgggcct | | | |
| MyD88 | atggctgcaggaggtcccggcgcggggtctgcg | 1025 | MAAGGPGAGSAAPVSSTSSLPLA | 1026 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQADP | |
| | gttcttgaacgtgcggacacaggtggcggccga | | TGRLLDAWQGRPGASVGRLLDLL | |
| | ctggaccgcgctggcggaggagatggactttga | | TKLGRDDVLLELGPSIEEDCQKYIL | |
| | gtacttggagatccggcaactggagacacaagc | | KQQQEEAEKPLQVAAVDSSVPRT | |
| | ggaccccactggcaggctgctggacgcctggc | | AELAGITTLDDPLGHMPERFDAFIC | |
| | agggacgccctggcgcctctgtaggccgactgc | | YCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgctatt | | | |
| | gccccagcgacatc | | | |
| Linker | gtcgag | 1027 | VE | 1028 |
| CD40 | aaaaaggtggccaagaagccaaccaataagg | 1029 | KKVAKKPTNKAPHPKQEPQEINFP | 1030 |
| | ccccccaccccaagcaggagccccaggagat | | DDLPGSNTAAPVQETLHGCQPVT | |
| | caattttcccgacgatcttcctggctccaacactgc | | QEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 1031 | VE | 1032 |
| FKBP_{V}_{^{′}} | GGcGTcCAaGTcGAaACcATtagtCCc | 1033 | GVQVETISPGDGRTFPKRGQTCV | 1034 |
| | GGcGAtGGcaGaACaTTtCCtAAaaG | | VHYTGMLEDGKKVDSSRDRNKPF | |
| | gGGaCAaACaTGtGTcGTcCAtTAtA | | KFMLGKQEVIRGWEEGVAQMSVG | |
| | CaGGcATGtTgGAgGAcGGcAAaAA | | QRAKLTISPDYAYGATGHPGIIPPH | |
| | gGTgGAcagtagtaGaGAtcGcAAtAAa | | ATLVFDVELLKLE | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cGGaATtATtCCcCCtCAcGCtACctTg | | | |
| | GTgTTtGAcGTcGAaCTgtTgAAgCTc | | | |
| | GAa | | | |
| Linker | gtcgag | 1035 | VE | 1036 |
| FKBP_{V} | ggagtgcaggtggagactatctccccaggagac | 1037 | GVQVETISPGDGRTFPKRGQTCV | 1038 |
| | gggcgcaccttccccaagcgcggccagacctg | | VHYTGMLEDGKKVDSSRDRNKPF | |
| | cgtggtgcactacaccgggatgcttgaagatgg | | KFMLGKQEVIRGVVEEGVAQMSVG | |
| | aaagaaagttgattcctcccgggacagaaaca | | QRAKLTISPDYAYGATGHPGIIPPH | |
| | agccctttaagtttatgctaggcaagcaggaggt | | ATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 1039 | stop | |

APPENDIX 3
pBP1310—pSFG.FRB.FKBP.ΔC9.T2A-ΔCD19

| | | SEQ | | SEQ |
|---|---|---|---|---|
| Fragment | Nucleotide | ID NO: | Peptide | ID NO: |
| Leader | ATGCtcgag | 1040 | MLE | 1041 |
| peptide | | | | |
| FRB | gaaatgTGGCATGAAGGGTTGGAAG | 1042 | EMWHEGLEEASRLYFGERNVKG | 1043 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGT | 1044 | SGGGSG | 1045 |
| FKBP wt | GGcGTcCAaGTcGAaACcATtagtCCc | 1046 | GVQVETISPGDGRTFPKRGQTCV | 1047 |
| | GGcGAtGGcaGaACaTTtCCtAAaaG | | VHYTGMLEDGKKFDSSRDRNKPF | |
| | gGGaCAaACaTGtGTcGTcCAtTAtA | | KFMLGKQEVIRGVVEEGVAQMSVG | |
| | CaGGcATGtTgGAgGAcGGcAAaAA | | QRAKLTISPDYAYGATGHPGIIPPH | |
| | gTTCGAcagtagtaGaGAtcGcAAtAAa | | ATLVFDVELLKL | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cggaattATtCCcCCtCAcGCtACctTgG | | | |
| | TgTTtGAcGTcGAaCTgtTgAAgCTc | | | |
| Linker | TCGGGGGGCGGATCAGGAGTCG | 1048 | SGGGSVD | 1049 |
| | AC | | | |
| Δcaspase9 | GGATTTGGTGATGTCGGTGCTCT | 1050 | GFGDVGALESLRGNADLAYILSME | 1051 |
| | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFNFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGACCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ccgcGG | 1052 | PR | 1053 |
| T2A | GAAGGCCGAGGGAGCCTGCTGA | 1054 | EGRGSLLTCGDVEENPGP | 1055 |
| | CATGTGGCGATGTGGAGGAAAAC | | | |
| | CCAGGACCA | | | |
| ACD19 | ATGCCACCACCTCGCCTGCTGTT | 1056 | MPPPRLLFFLLFLTPMEVRPEEPL | 1057 |
| | CTTTCTGCTGTTCCTGACACCTAT | | VVKVEEGDNAVLQCLKGTSDGPT | |
| | GGAGGTGCGACCTGAGGAACCAC | | QQLTWSRESPLKPFLKLSLGLPGL | |
| | TGGTCGTGAAGGTCGAGGAAGGC | | GIHMRPLAIWLFIFNVSQQMGGFY | |
| | GACAATGCCGTGCTGCAGTGCCT | | LCQPGPPSEKAWQPGWTVNVEG | |
| | GAAAGGCACTTCTGATGGGCCAA | | SGELFRWNVSDLGGLGCGLKNRS | |
| | CTCAGCAGCTGACCTGGTCCAGG | | SEGPSSPSGKLMSPKLYVWAKDR | |
| | GAGTCTCCCCTGAAGCCTTTTCTG | | PEIWEGEPPCLPPRDSLNQSLSQD | |
| | AAACTGAGCCTGGGACTGCCAGG | | LTMAPGSTLWLSCGVPPDSVSRG | |
| | ACTGGGAATCCACATGCGCCCTC | | PLSWTHVHPKGPKSLLSLELKDDR | |
| | TGGCTATCTGGCTGTTCATCTTCA | | PARDMWVMETGLLLPRATAQDAG | |
| | ACGTGAGCCAGCAGATGGGAGGA | | KYYCHRGNLTMSFHLEITARPVLW | |
| | TTCTACCTGTGCCAGCCAGGACC | | HWLLRTGGWKVSAVTLAYLIFCLC | |
| | ACCATCCGAGAAGGCCTGGCAGC | | SLVGILHLQRALVLRRKRKRMTDP | |
| | CTGGATGGACCGTCAACGTGGAG | | TRRF | |
| | GGGTCTGGAGAACTGTTTAGGTG | | | |
| | GAATGTGAGTGACCTGGGAGGAC | | | |
| | TGGGATGTGGGCTGAAGAACCGC | | | |
| | TCCTCTGAAGGCCCAAGTTCACC | | | |
| | CTCAGGGAAGCTGATGAGCCCAA | | | |
| | AACTGTACGTGTGGGCCAAAGAT | | | |
| | CGGCCCGAGATCTGGGAGGGAG | | | |
| | AACCTCCATGCCTGCCACCTAGA | | | |
| | GACAGCCTGAATCAGAGTCTGTC | | | |
| | ACAGGATCTGACAATGGCCCCCG | | | |
| | GGTCCACTCTGTGGCTGTCTTGT | | | |
| | GGAGTCCCACCCGACAGCGTGTC | | | |
| | CAGAGGCCCTCTGTCCTGGACCC | | | |
| | ACGTGCATCCTAAGGGGCCAAAA | | | |
| | AGTCTGCTGTCACTGGAACTGAA | | | |
| | GGACGATCGGCCTGCCAGAGACA | | | |
| | TGTGGGTCATGGAGACTGGACTG | | | |
| | CTGCTGCCACGAGCAACCGCACA | | | |
| | GGATGCTGGAAAATACTATTGCCA | | | |
| | CCGGGGCAATCTGACAATGTCCT | | | |
| | TCCATCTGGAGATCACTGCAAGG | | | |
| | CCCGTGCTGTGGCACTGGCTGCT | | | |
| | GCGAACCGGAGGATGGAAGGTCA | | | |
| | GTGCTGTGACACTGGCATATCTG | | | |
| | ATCTTTTGCCTGTGCTCCCTGGTG | | | |
| | GGCATTCTGCATCTGCAGAGAGC | | | |
| | CCTGGTGCTGCGGAGAAAGAGAA | | | |
| | AGAGAATGACTGACCCAACAAGA | | | |
| | AGGTTT | | | |
| STOP | TGA | 1058 | stop | |

APPENDIX 4
pBP1311—pSFG.FKBP.FRB.ΔC9.T2A-ΔCD19

| | | SEQ | | SEQ |
|---|---|---|---|---|
| Fragment | Nucleotide | ID NO: | Peptide | ID NO: |
| Leader | ATGCtcgag | 1059 | MLE | 1060 |
| peptide | | | | |
| FKBP wt | GGcGTcCAaGTcGAaACcATtagtCCc | 1061 | GVQVETISPGDGRTFPKRGQTCV | 1062 |
| | GGcGAtGGcaGaACaTTtCCtAAaaG | | VHYTGMLEDGKKFDSSRDRNKPF | |
| | gGGaCAaACaTGtGTcGTcCAtTAtA | | KFMLGKQEVIRGVVEEGVAQMSVG | |
| | CaGGcATGtTgGAgGAcGGcAAaAA | | QRAKLTISPDYAYGATGHPGIIPPH | |
| | gTTCGAcagtagtaGaGAtcGcAAtAAa | | ATLVFDVELLKL | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cggaattATtCCcCCtCAcGCtACctTgG | | | |
| | TgTTtGAcGTcGAaCTgtTgAAgCTc | | | |
| Linker | TCGGGGGGCGGATCAGG | 1063 | SGGGS | 1064 |
| FRB | gaaatgTGGCATGAAGGGTTGGAAG | 1065 | EMVVHEGLEEASRLYFGERNVKG | 1066 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTGTCGA | 1067 | SGGGSGVD | 1068 |
| | C | | | |
| Δcaspase9 | GGATTTGGTGATGTCGGTGCTCT | 1069 | GFGDVGALESLRGNADLAYILSME | 1070 |
| | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFNFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGACCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ccgcGG | 1071 | PR | 1072 |
| T2A | GAAGGCCGAGGGAGCCTGCTGA | 1073 | EGRGSLLTCGDVEENPGP | 1074 |
| | CATGTGGCGATGTGGAGGAAAAC | | | |
| | CCAGGACCA | | | |
| ΔCD19 | ATGCCACCACCTCGCCTGCTGTT | 1075 | MPPPRLLFFLLFLTPMEVRPEEPL | 1076 |
| | CTTTCTGCTGTTCCTGACACCTAT | | VVKVEEGDNAVLQCLKGTSDGPT | |
| | GGAGGTGCGACCTGAGGAACCAC | | QQLTWSRESPLKPFLKLSLGLPGL | |
| | TGGTCGTGAAGGTCGAGGAAGGC | | GIHMRPLAIVVLFIFNVSQQMGGFY | |
| | GACAATGCCGTGCTGCAGTGCCT | | LCQPGPPSEKAWQPGVVTVNVEG | |
| | GAAAGGCACTTCTGATGGGCCAA | | SGELFRWNVSDLGGLGCGLKNRS | |
| | CTCAGCAGCTGACCTGGTCCAGG | | SEGPSSPSGKLMSPKLYVWAKDR | |
| | GAGTCTCCCCTGAAGCCTTTTCTG | | PEIVVEGEPPCLPPRDSLNQSLSQD | |
| | AAACTGAGCCTGGGACTGCCAGG | | LTMAPGSTLWLSCGVPPDSVSRG | |
| | ACTGGGAATCCACATGCGCCCTC | | PLSVVTHVHPKGPKSLLSLELKDDR | |
| | TGGCTATCTGGCTGTTCATCTTCA | | PARDMVVVMETGLLLPRATAQDAG | |
| | ACGTGAGCCAGCAGATGGGAGGA | | KYYCHRGNLTMSFHLEITARPVLW | |
| | TTCTACCTGTGCCAGCCAGGACC | | HVVLLRTGGVVKVSAVTLAYLIFCLC | |
| | ACCATCCGAGAAGGCCTGGCAGC | | SLVGILHLQRALVLRRKRKRMTDP | |
| | CTGGATGGACCGTCAACGTGGAG | | TRRF | |
| | GGGTCTGGAGAACTGTTTAGGTG | | | |
| | GAATGTGAGTGACCTGGGAGGAC | | | |
| | TGGGATGTGGGCTGAAGAACCGC | | | |
| | TCCTCTGAAGGCCCAAGTTCACC | | | |
| | CTCAGGGAAGCTGATGAGCCCAA | | | |
| | AACTGTACGTGTGGGCCAAAGAT | | | |
| | CGGCCCGAGATCTGGGAGGGAG | | | |
| | AACCTCCATGCCTGCCACCTAGA | | | |
| | GACAGCCTGAATCAGAGTCTGTC | | | |
| | ACAGGATCTGACAATGGCCCCCG | | | |
| | GGTCCACTCTGTGGCTGTCTTGT | | | |
| | GGAGTCCCACCCGACAGCGTGTC | | | |
| | CAGAGGCCCTCTGTCCTGGACCC | | | |
| | ACGTGCATCCTAAGGGGCCAAAA | | | |
| | AGTCTGCTGTCACTGGAACTGAA | | | |
| | GGACGATCGGCCTGCCAGAGACA | | | |
| | TGTGGGTCATGGAGACTGGACTG | | | |
| | CTGCTGCCACGAGCAACCGCACA | | | |
| | GGATGCTGGAAAATACTATTGCCA | | | |
| | CCGGGGCAATCTGACAATGTCCT | | | |
| | TCCATCTGGAGATCACTGCAAGG | | | |
| | CCCGTGCTGTGGCACTGGCTGCT | | | |
| | GCGAACCGGAGGATGGAAGGTCA | | | |
| | GTGCTGTGACACTGGCATATCTG | | | |
| | ATCTTTTGCCTGTGCTCCCTGGTG | | | |
| | GGCATTCTGCATCTGCAGAGAGC | | | |
| | CCTGGTGCTGCGGAGAAAGAGAA | | | |
| | AGAGAATGACTGACCCAACAAGA | | | |
| | AGGTTT | | | |
| STOP | TGA | 1077 | stop | |

APPENDIX 5
pBP1316—pSFG-FKBP.FRB_{L}.ΔC9.T2A-αPSCA.Q.CD8stm.ζ.P2A-iMC

| | | SEQ | | SEQ |
|---|---|---|---|---|
| Fragment | Nucleotide | ID NO: | Peptide | ID NO: |
| Leader | ATGCtcgagcaattg | 1078 | MLEQL | 1079 |
| peptide | | | | |
| FKBP″wt | GGcGTGCAaGTGGAaACTATaAGC | 1080 | GVQVETISPGDGRTFPKRGQTCV | 1081 |
| | CCgGGAGAcGGCcGcACATTtCCC | | VHYTGMLEDGKKFDSSRDRNKPF | |
| | AAgAGAGGcCAGACcTGCGTgGTG | | KFMLGKQEVIRGVVEEGVAQMSVG | |
| | CAcTATACaGGAATGCTGGAgGAC | | QRAKLTISPDYAYGATGHPGIIPPH | |
| | GGgAAGAAaTTCGAtAGCtcCCGGG | | ATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 1082 | ggql | 1083 |
| FRB_{L} | gaaatgTGGCATGAAGGGTTGGAAG | 1084 | EMVVHEGLEEASRLYFGERNVKG | 1085 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLLQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGcttCAGGCCTGGGACC | | | |
| | TTTATTACCATGTGTTCAGGCGGA | | | |
| | TCAGTAAG | | | |
| Linker | TCAGGCGGTGGCTCAGGTccatgg | 1086 | SGGGSGPW | 1087 |
| Δcaspase9 | GGATTTGGTGATGTCGGTGCTCT | 1088 | GFGDVGALESLRGNADLAYILSME | 1089 |
| | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFNFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ggatctggaccgcGG | 1090 | GSGPR | 1091 |
| T2A | GAAGGCCGAGGGAGCCTGCTGA | 1092 | EGRGSLLTCGDVEENPGP | 1093 |
| | CATGTGGCGATGTGGAGGAAAAC | | | |
| | CCAGGACCA | | | |
| Linker | CCATGG | 1094 | PW | 1095 |
| Signal | ATGGAGTTTGGACTTTCTTGGTTG | 1096 | MEFGLSVVLFLVAILKGVQCSR | 1097 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1098 | DIQLTQSPSTLSASMGDRVTITCSA | 1099 |
| VL | ATCTACACTCAGCGCTAGCATGG | | SSSVRFIHVVYQQKPGKAPKRLIYD | |
| | GGGACAGGGTCACAATCACGTGC | | TSKLASGVPSRFSGSGSGTDFTLT | |
| | TCTGCCTCAAGTTCCGTTAGGTTT | | ISSLQPEDFATYYCQQWGSSPFTF | |
| | ATCCATTGGTATCAGCAGAAACCT | | GQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1100 | gggsgggg | 1101 |
| | GC | | | |
| PSCA(A11) | GAGGTGCAGCTCGTGGAGTATGG | 1102 | EVQLVEYGGGLVQPGGSLRLSCA | 1103 |
| VH | CGGGGGCCTGGTGCAGCCTGGG | | ASGFNIKDYYIHVVVRQAPGKGLE | |
| | GGTAGTCTGAGGCTCTCCTGCGC | | VVVAWIDPENGDTEFVPKFQGRAT | |
| | TGCCTCTGGCTTTAACATTAAAGA | | MSADTSKNTAYLQMNSLRAEDTA | |
| | CTACTACATACATTGGGTGCGGC | | VYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1104 | gs | 1105 |
| CD34 | GAACTTCCTACTCAGGGGACTTTC | 1106 | ELPTQGTFSNVSTNVS | 1107 |
| epitope | TCAAACGTTAGCACAAACGTAAGT | | | |
| CD8 stalk | CCCGCCCCAAGACCCCCCACACC | 1108 | PAPRPPTPAPTIASQPLSLRPEAC | 1109 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1110 | IYIWAPLAGTCGVLLLSLVITLYCNH | 1111 |
| trans- | GGCACCTGTGGAGTCCTTCTGCT | | RNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1112 | VD | 1113 |
| CD3ζ | AGAGTGAAGTTCAGCAGGAGCGC | 1114 | RVKFSRSADAPAYQQGQNQLYNE | 1115 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPPR | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 1116 | GTRGSG | 1117 |
| P2A | GCTACTAACTTCAGCCTGCTGAA | 1118 | ATNFSLLKQAGDVEENPGP | 1119 |
| | GCAGGCTGGAGACGTGGAGGAG | | | |
| | AACcccgggcct | | | |
| MyD88 | atggctgcaggaggtcccggcgcggggtctgcg | 1120 | MAAGGPGAGSAAPVSSTSSLPLA | 1121 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQADP | |
| | gttcttgaacgtgcggacacaggtggcggccga | | TGRLLDAWQGRPGASVGRLLDLL | |
| | ctggaccgcgctggcggaggagatggactttga | | TKLGRDDVLLELGPSIEEDCQKYIL | |
| | gtacttggagatccggcaactggagacacaagc | | KQQQEEAEKPLQVAAVDSSVPRT | |
| | ggaccccactggcaggctgctggacgcctggc | | AELAGITTLDDPLGHMPERFDAFIC | |
| | agggacgccctggcgcctctgtaggccgactgc | | YCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgctatt | | | |
| | gccccagcgacatc | | | |
| Linker | gtcgag | 1122 | VE | 1123 |
| CD40 | aaaaaggtggccaagaagccaaccaataagg | 1124 | KKVAKKPTNKAPHPKQEPQEINFP | 1125 |
| | ccccccaccccaagcaggagccccaggagat | | DDLPGSNTAAPVQETLHGCQPVT | |
| | caattttcccgacgatcttcctggctccaacactgc | | QEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 1126 | VE | 1127 |
| FKBP_{V}_{^{′}} | GGcGTcCAaGTcGAaACcATtagtCCc | 1128 | GVQVETISPGDGRTFPKRGQTCV | 1129 |
| | GGcGAtGGcaGaACaTTtCCtAAaaG | | VHYTGMLEDGKKVDSSRDRNKPF | |
| | gGGaCAaACaTGtGTcGTcCAtTAtA | | KFMLGKQEVIRGVVEEGVAQMSVG | |
| | CaGGcATGtTgGAgGAcGGcAAaAA | | QRAKLTISPDYAYGATGHPGIIPPH | |
| | gGTgGAcagtagtaGaGAtcGcAAtAAa | | ATLVFDVELLKLE | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cGGaATtATtCCcCCtCAcGCtACctTg | | | |
| | GTgTTtGAcGTcGAaCTgtTgAAgCTc | | | |
| | GAa | | | |
| Linker | gtcgag | 1130 | VE | 1131 |
| FKBP_{V} | ggagtgcaggtggagactatctccccaggagac | 1132 | GVQVETISPGDGRTFPKRGQTCV | 1133 |
| | gggcgcaccttccccaagcgcggccagacctg | | VHYTGMLEDGKKVDSSRDRNKPF | |
| | cgtggtgcactacaccgggatgcttgaagatgg | | KFMLGKQEVIRGVVEEGVAQMSVG | |
| | aaagaaagttgattcctcccgggacagaaaca | | QRAKLTISPDYAYGATGHPGIIPPH | |
| | agccctttaagtttatgctaggcaagcaggaggt | | ATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 1134 | stop | |

APPENDIX 6
pBP1317—pSFG-FKBP.FRB.ΔC9_{Q}.T2A-αPSCA.Q.CD8stm.ζ.P2A-iMC

| | | SEQ | | SEQ |
|---|---|---|---|---|
| | | ID | | ID |
| Fragment | Nucleotide | NO: | Peptide | NO: |
| Leader | ATGCtcgagcaattg | 1135 | MLEQL | 1136 |
| peptide | | | | |
| FKBP″wt | GGcGTGCAaGTGGAaACTATaAGC | 1137 | GVQVETISPGDGRTFPKRGQTCV | 1138 |
| | CCgGGAGAcGGCcGcACATTtCCC | | VHYTGMLEDGKKFDSSRDRNKPF | |
| | AAgAGAGGcCAGACcTGCGTgGTG | | KFMLGKQEVIRGVVEEGVAQMSVG | |
| | CAcTATACaGGAATGCTGGAgGAC | | QRAKLTISPDYAYGATGHPGIIPPH | |
| | GGgAAGAAaTTCGAtAGCtcCCGGG | | ATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 1139 | ggql | 1140 |
| FRB | gaaatgTGGCATGAAGGGTTGGAAG | 1141 | EMVVHEGLEEASRLYFGERNVKG | 1142 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMKS | |
| | TGAGGTTCTTGAACCTCTGCACG | | GNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 1143 | SGGGSGPW | 1144 |
| Δcaspase- | GGATTTGGTGATGTCGGTGCTCT | 1145 | GFGDVGALESLRGNADLAYILSME | 1146 |
| 9_{Q}(N405Q) | TGAGAGTTTGAGGGGAAATGCAG | | PCGHCLIINNVNFCRESGLRTRTG | |
| | ATTTGGCTTACATCCTGAGCATGG | | SNIDCEKLRRRFSSLHFMVEVKGD | |
| | AGCCCTGTGGCCACTGCCTCATT | | LTAKKMVLALLELARQDHGALDCC | |
| | ATCAACAATGTGAACTTCTGCCGT | | VVVILSHGCQASHLQFPGAVYGTD | |
| | GAGTCCGGGCTCCGCACCCGCA | | GCPVSVEKIVNIFNGTSCPSLGGK | |
| | CTGGCTCCAACATCGACTGTGAG | | PKLFFIQACGGEQKDHGFEVASTS | |
| | AAGTTGCGGCGTCGCTTCTCCTC | | PEDESPGSNPEPDATPFQEGLRT | |
| | GCTGCATTTCATGGTGGAGGTGA | | FDQLDAISSLPTPSDIFVSYSTFPG | |
| | AGGGCGACCTGACTGCCAAGAAA | | FVSWRDPKSGSVVYVETLDDIFEQ | |
| | ATGGTGCTGGCTTTGCTGGAGCT | | WAHSEDLQSLLLRVANAVSVKGIY | |
| | GGCGCgGCAGGACCACGGTGCTC | | KQMPGCFQFLRKKLFFKTSASRA | |
| | TGGACTGCTGCGTGGTGGTCATT | | | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTc | | | |
| | AaTTCCTCCGGAAAAAACTTTTCT | | | |
| | TTAAAACATCAGCTAGCAGAGCC | | | |
| Linker | ggatctggaccgcGG | 1147 | GSGPR | 1148 |
| T2A | GAAGGCCGAGGGAGCCTGCTGA | 1149 | EGRGSLLTCGDVEENPGP | 1150 |
| | CATGTGGCGATGTGGAGGAAAAC | | | |
| | CCAGGACCA | | | |
| Linker | CCATGG | 1151 | PW | 1152 |
| Signal | ATGGAGTTTGGACTTTCTTGGTTG | 1153 | MEFGLSVVLFLVAILKGVQCSR | 1154 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1155 | DIQLTQSPSTLSASMGDRVTITCSA | 1156 |
| VL | ATCTACACTCAGCGCTAGCATGG | | SSSVRFIHVVYQQKPGKAPKRLIYD | |
| | GGGACAGGGTCACAATCACGTGC | | TSKLASGVPSRFSGSGSGTDFTLT | |
| | TCTGCCTCAAGTTCCGTTAGGTTT | | ISSLQPEDFATYYCQQWGSSPFTF | |
| | ATCCATTGGTATCAGCAGAAACCT | | GQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1157 | gggsgggg | 1158 |
| | GC | | | |
| PSCA(A11) | GAGGTGCAGCTCGTGGAGTATGG | 1159 | EVQLVEYGGGLVQPGGSLRLSCA | 1160 |
| VH | CGGGGGCCTGGTGCAGCCTGGG | | ASGFNIKDYYIHVVVRQAPGKGLE | |
| | GGTAGTCTGAGGCTCTCCTGCGC | | VVVAWIDPENGDTEFVPKFQGRAT | |
| | TGCCTCTGGCTTTAACATTAAAGA | | MSADTSKNTAYLQMNSLRAEDTA | |
| | CTACTACATACATTGGGTGCGGC | | VYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1161 | gs | 1162 |
| CD34 | GAACTTCCTACTCAGGGGACTTTC | 1163 | ELPTQGTFSNVSTNVS | 1164 |
| epitope | TCAAACGTTAGCACAAACGTAAGT | | | |
| CD8 stalk | CCCGCCCCAAGACCCCCCACACC | 1165 | PAPRPPTPAPTIASQPLSLRPEAC | 1166 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1167 | IYIWAPLAGTCGVLLLSLVITLYCNH | 1168 |
| trans- | GGCACCTGTGGAGTCCTTCTGCT | | RNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1169 | VD | 1170 |
| CD3ζ | AGAGTGAAGTTCAGCAGGAGCGC | 1171 | RVKFSRSADAPAYQQGQNQLYNE | 1172 |
| | AGACGCCCCCGCGTACCAGCAG | | LNLGRREEYDVLDKRRGRDPEMG | |
| | GGCCAGAACCAGCTCTATAACGA | | GKPRRKNPQEGLYNELQKDKMAE | |
| | GCTCAATCTAGGACGAAGAGAGG | | AYSEIGMKGERRRGKGHDGLYQG | |
| | AGTACGATGTTTTGGACAAGAGA | | LSTATKDTYDALHMQALPPR | |
| | CGTGGCCGGGACCCTGAGATGG | | | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 1173 | GTRGSG | 1174 |
| P2A | GCTACTAACTTCAGCCTGCTGAA | 1175 | ATNFSLLKQAGDVEENPGP | 1176 |
| | GCAGGCTGGAGACGTGGAGGAG | | | |
| | AACcccgggcct | | | |
| MyD88 | atggctgcaggaggtcccggcgcggggtctgcg | 1177 | MAAGGPGAGSAAPVSSTSSLPLA | 1178 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQADP | |
| | gttcttgaacgtgcggacacaggtggcggccga | | TGRLLDAWQGRPGASVGRLLDLL | |
| | ctggaccgcgctggcggaggagatggactttga | | TKLGRDDVLLELGPSIEEDCQKYIL | |
| | gtacttggagatccggcaactggagacacaagc | | KQQQEEAEKPLQVAAVDSSVPRT | |
| | ggaccccactggcaggctgctggacgcctggc | | AELAGITTLDDPLGHMPERFDAFIC | |
| | agggacgccctggcgcctctgtaggccgactgc | | YCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgcta | | | |
| | ttgccccagcgacatc | | | |
| Linker | gtcgag | 1179 | VE | 1180 |
| CD40 | aaaaaggtggccaagaagccaaccaataagg | 1181 | KKVAKKPTNKAPHPKQEPQEINFP | 1182 |
| | ccccccaccccaagcaggagccccaggagat | | DDLPGSNTAAPVQETLHGCQPVT | |
| | caattttcccgacgatcttcctggctccaacactgc | | QEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 1183 | VE | 1184 |
| FKBP_{V}_{^{′}} | GGcGTcCAaGTcGAaACcATtagtCCc | 1185 | GVQVETISPGDGRTFPKRGQTCV | 1186 |
| | GGcGAtGGcaGaACaTTtCCtAAaaG | | VHYTGMLEDGKKVDSSRDRNKPF | |
| | gGGaCAaACaTGtGTcGTcCAtTAtA | | KFMLGKQEVIRGVVEEGVAQMSVG | |
| | CaGGcATGtTgGAgGAcGGcAAaAA | | QRAKLTISPDYAYGATGHPGIIPPH | |
| | gGTgGAcagtagtaGaGAtcGcAAtAAa | | ATLVFDVELLKLE | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cGGaATtATtCCcCCtCAcGCtACctTg | | | |
| | GTgTTtGAcGTcGAaCTgtTgAAgCTc | | | |
| | GAa | | | |
| Linker | gtcgag | 1187 | VE | 1188 |
| FKBP_{V} | ggagtgcaggtggagactatctccccaggagac | 1189 | GVQVETISPGDGRTFPKRGQTCV | 1190 |
| | gggcgcaccttccccaagcgcggccagacctg | | VHYTGMLEDGKKVDSSRDRNKPF | |
| | cgtggtgcactacaccgggatgcttgaagatgg | | KFMLGKQEVIRGVVEEGVAQMSVG | |
| | aaagaaagttgattcctcccgggacagaaaca | | QRAKLTISPDYAYGATGHPGIIPPH | |
| | agccctttaagtttatgctaggcaagcaggaggt | | ATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 1191 | stop | |

APPENDIX 7
pBP1319—pSFG-FKBP.FRB.ΔC9.T2A-αPSCA.Q.CD8stm.ζ.P2A-MC.FKBP_{V}

| | | SEQ | | SEQ |
|---|---|---|---|---|
| | | ID | | ID |
| Fragment | Nucleotide | NO: | Peptide | NO: |
| Leader | ATGCtcgagcaattg | 1192 | MLEQL | 1193 |
| peptide | | | | |
| FKBP″wt | GGcGTGCAaGTGGAaACTATaAGC | 1194 | GVQVETISPGDGRTFPKRGQTCV | 1195 |
| | CCgGGAGAcGGCcGcACATTtCCC | | VHYTGMLEDGKKFDSSRDRNKP | |
| | AAgAGAGGcCAGACcTGCGTgGTG | | FKFMLGKQEVIRGWEEGVAQMS | |
| | CAcTATACaGGAATGCTGGAgGAC | | VGQRAKLTISPDYAYGATGHPGII | |
| | GGgAAGAAaTTCGAtAGCtcCCGGG | | PPHATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 1196 | ggql | 1197 |
| FRB | gaaatgTGGCATGAAGGGTTGGAAG | 1198 | EMWHEGLEEASRLYFGERNVKG | 1199 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMK | |
| | TGAGGTTCTTGAACCTCTGCACG | | SGNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 1200 | SGGGSGPW | 1201 |
| Δcaspase- | GGATTTGGTGATGTCGGTGCTCT | 1202 | GFGDVGALESLRGNADLAYILSM | 1203 |
| 9_{Q} | TGAGAGTTTGAGGGGAAATGCAG | | EPCGHCLIINNVNFCRESGLRTRT | |
| | ATTTGGCTTACATCCTGAGCATGG | | GSNIDCEKLRRRFSSLHFMVEVK | |
| | AGCCCTGTGGCCACTGCCTCATT | | GDLTAKKMVLALLELARQDHGAL | |
| | ATCAACAATGTGAACTTCTGCCGT | | DCCVVVILSHGCQASHLQFPGAV | |
| | GAGTCCGGGCTCCGCACCCGCA | | YGTDGCPVSVEKIVNIFNGTSCPS | |
| | CTGGCTCCAACATCGACTGTGAG | | LGGKPKLFFIQACGGEQKDHGFE | |
| | AAGTTGCGGCGTCGCTTCTCCTC | | VASTSPEDESPGSNPEPDATPFQ | |
| | GCTGCATTTCATGGTGGAGGTGA | | EGLRTFDQLDAISSLPTPSDIFVS | |
| | AGGGCGACCTGACTGCCAAGAAA | | YSTFPGFVSVVRDPKSGSVVYVET | |
| | ATGGTGCTGGCTTTGCTGGAGCT | | LDDIFEQWAHSEDLQSLLLRVAN | |
| | GGCGCgGCAGGACCACGGTGCTC | | AVSVKGIYKQMPGCFQFLRKKLF | |
| | TGGACTGCTGCGTGGTGGTCATT | | FKTSASRA | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTc | | | |
| | AaTTCCTCCGGAAAAAACTTTTCT | | | |
| | TTAAAACATCAGCTAGCAGAGCC | | | |
| Linker | ggatctggaccgcGG | 1204 | GSGPR | 1205 |
| T2A | GAAGGCCGAGGGAGCCTGCTGA | 1206 | EGRGSLLTCGDVEENPGP | 1207 |
| | CATGTGGCGATGTGGAGGAAAAC | | | |
| | CCAGGACCA | | | |
| Linker | CCATGG | 1208 | PW | 1209 |
| Signal | ATGGAGTTTGGACTTTCTTGGTTG | 1210 | MEFGLSVVLFLVAILKGVQCSR | 1211 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1212 | DIQLTQSPSTLSASMGDRVTITCS | 1213 |
| VL | ATCTACACTCAGCGCTAGCATGG | | ASSSVRFIHVVYQQKPGKAPKRLI | |
| | GGGACAGGGTCACAATCACGTGC | | YDTSKLASGVPSRFSGSGSGTDF | |
| | TCTGCCTCAAGTTCCGTTAGGTTT | | TLTISSLQPEDFATYYCQQWGSS | |
| | ATCCATTGGTATCAGCAGAAACCT | | PFTFGQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1214 | gggsgggg | 1215 |
| | GC | | | |
| PSCA(A11) | GAGGTGCAGCTCGTGGAGTATGG | 1216 | EVQLVEYGGGLVQPGGSLRLSC | 1217 |
| VH | CGGGGGCCTGGTGCAGCCTGGG | | AASGFNIKDYYIHVVVRQAPGKGL | |
| | GGTAGTCTGAGGCTCTCCTGCGC | | EVVVAWIDPENGDTEFVPKFQGR | |
| | TGCCTCTGGCTTTAACATTAAAGA | | ATMSADTSKNTAYLQMNSLRAED | |
| | CTACTACATACATTGGGTGCGGC | | TAVYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1218 | gs | 1219 |
| CD34 | GAACTTCCTACTCAGGGGACTTTC | 1220 | ELPTQGTFSNVSTNVS | 1221 |
| epitope | TCAAACGTTAGCACAAACGTAAGT | | | |
| CD8 stalk | CCCGCCCCAAGACCCCCCACACC | 1222 | PAPRPPTPAPTIASQPLSLRPEAC | 1223 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1224 | IYIWAPLAGTCGVLLLSLVITLYCN | 1225 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1226 | VD | 1227 |
| CD3ζ | AGAGTGAAGTTCAGCAGGAGCGC | 1228 | RVKFSRSADAPAYQQGQNQLYN | 1229 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 1230 | GTRGSG | 1231 |
| P2A | GCTACTAACTTCAGCCTGCTGAA | 1232 | ATNFSLLKQAGDVEENPGP | 1233 |
| | GCAGGCTGGAGACGTGGAGGAG | | | |
| | AACcccgggcct | | | |
| MyD88 | atggctgcaggaggtcccggcgcggggtctgcg | 1234 | MAAGGPGAGSAAPVSSTSSLPLA | 1235 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQAD | |
| | gttcttgaacgtgcggacacaggtggcggccga | | PTGRLLDAWQGRPGASVGRLLD | |
| | ctggaccgcgctggcggaggagatggactttga | | LLTKLGRDDVLLELGPSIEEDCQK | |
| | gtacttggagatccggcaactggagacacaagc | | YILKQQQEEAEKPLQVAAVDSSV | |
| | ggaccccactggcaggctgctggacgcctggc | | PRTAELAGITTLDDPLGHMPERF | |
| | agggacgccctggcgcctctgtaggccgactgc | | DAFICYCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgcta | | | |
| | ttgccccagcgacatc | | | |
| Linker | gtcgag | 1236 | VE | 1237 |
| CD40 | aaaaaggtggccaagaagccaaccaataagg | 1238 | KKVAKKPTNKAPHPKQEPQEINF | 1239 |
| | ccccccaccccaagcaggagccccaggagat | | PDDLPGSNTAAPVQETLHGCQP | |
| | caattttcccgacgatcttcctggctccaacactgc | | VTQEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 1240 | VE | 1241 |
| FKBP_{V} | ggagtgcaggtggagactatctccccaggagac | 1242 | GVQVETISPGDGRTFPKRGQTCV | 1243 |
| | gggcgcaccttccccaagcgcggccagacctg | | VHYTGMLEDGKKVDSSRDRNKP | |
| | cgtggtgcactacaccgggatgcttgaagatgg | | FKFMLGKQEVIRGWEEGVAQMS | |
| | aaagaaagttgattcctcccgggacagaaaca | | VGQRAKLTISPDYAYGATGHPGII | |
| | agccctttaagtttatgctaggcaagcaggaggt | | PPHATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 1244 | stop | |

APPENDIX 8
pBP1320—pSFG-FKBP.FRB.ΔC9.T2A-αPSCA.Q.CD8stm.ζ.P2A-MC

| | | SEQ | | SEQ |
|---|---|---|---|---|
| | | ID | | ID |
| Fragment | Nucleotide | NO: | Peptide | NO: |
| Leader | ATGCtcgagcaattg | 1245 | MLEQL | 1246 |
| peptide | | | | |
| FKBP″wt | GGcGTGCAaGTGGAaACTATaAGC | 1247 | GVQVETISPGDGRTFPKRGQTCV | 1248 |
| | CCgGGAGAcGGCcGcACATTtCCC | | VHYTGMLEDGKKFDSSRDRNKP | |
| | AAgAGAGGcCAGACcTGCGTgGTG | | FKFMLGKQEVIRGWEEGVAQMS | |
| | CAcTATACaGGAATGCTGGAgGAC | | VGQRAKLTISPDYAYGATGHPGII | |
| | GGgAAGAAaTTCGAtAGCtcCCGGG | | PPHATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 1249 | ggql | 1250 |
| FRB | gaaatgTGGCATGAAGGGTTGGAAG | 1251 | EMWHEGLEEASRLYFGERNVKG | 1252 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMK | |
| | TGAGGTTCTTGAACCTCTGCACG | | SGNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 1253 | SGGGSGPW | 1254 |
| Δcaspase- | GGATTTGGTGATGTCGGTGCTCT | 1255 | GFGDVGALESLRGNADLAYILSM | 1256 |
| 9_{Q} | TGAGAGTTTGAGGGGAAATGCAG | | EPCGHCLIINNVNFCRESGLRTRT | |
| | ATTTGGCTTACATCCTGAGCATGG | | GSNIDCEKLRRRFSSLHFMVEVK | |
| | AGCCCTGTGGCCACTGCCTCATT | | GDLTAKKMVLALLELARQDHGAL | |
| | ATCAACAATGTGAACTTCTGCCGT | | DCCVVVILSHGCQASHLQFPGAV | |
| | GAGTCCGGGCTCCGCACCCGCA | | YGTDGCPVSVEKIVNIFNGTSCPS | |
| | CTGGCTCCAACATCGACTGTGAG | | LGGKPKLFFIQACGGEQKDHGFE | |
| | AAGTTGCGGCGTCGCTTCTCCTC | | VASTSPEDESPGSNPEPDATPFQ | |
| | GCTGCATTTCATGGTGGAGGTGA | | EGLRTFDQLDAISSLPTPSDIFVS | |
| | AGGGCGACCTGACTGCCAAGAAA | | YSTFPGFVSVVRDPKSGSVVYVET | |
| | ATGGTGCTGGCTTTGCTGGAGCT | | LDDIFEQWAHSEDLQSLLLRVAN | |
| | GGCGCgGCAGGACCACGGTGCTC | | AVSVKGIYKQMPGCFQFLRKKLF | |
| | TGGACTGCTGCGTGGTGGTCATT | | FKTSASRA | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTc | | | |
| | AaTTCCTCCGGAAAAAACTTTTCT | | | |
| | TTAAAACATCAGCTAGCAGAGCC | | | |
| Linker | ggatctggaccgcGG | 1257 | GSGPR | 1258 |
| T2A | GAAGGCCGAGGGAGCCTGCTGA | 1259 | EGRGSLLTCGDVEENPGP | 1260 |
| | CATGTGGCGATGTGGAGGAAAAC | | | |
| | CCAGGACCA | | | |
| Linker | CCATGG | 1261 | PW | 1262 |
| Signal | ATGGAGTTTGGACTTTCTTGGTTG | 1263 | MEFGLSVVLFLVAILKGVQCSR | 1264 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1265 | DIQLTQSPSTLSASMGDRVTITCS | 1266 |
| VL | ATCTACACTCAGCGCTAGCATGG | | ASSSVRFIHVVYQQKPGKAPKRLI | |
| | GGGACAGGGTCACAATCACGTGC | | YDTSKLASGVPSRFSGSGSGTDF | |
| | TCTGCCTCAAGTTCCGTTAGGTTT | | TLTISSLQPEDFATYYCQQWGSS | |
| | ATCCATTGGTATCAGCAGAAACCT | | PFTFGQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1267 | gggsgggg | 1268 |
| | GC | | | |
| PSCA(A11) | GAGGTGCAGCTCGTGGAGTATGG | 1269 | EVQLVEYGGGLVQPGGSLRLSC | 1270 |
| VH | CGGGGGCCTGGTGCAGCCTGGG | | AASGFNIKDYYIHVVVRQAPGKGL | |
| | GGTAGTCTGAGGCTCTCCTGCGC | | EVVVAWIDPENGDTEFVPKFQGR | |
| | TGCCTCTGGCTTTAACATTAAAGA | | ATMSADTSKNTAYLQMNSLRAED | |
| | CTACTACATACATTGGGTGCGGC | | TAVYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1271 | gs | 1272 |
| CD34 | GAACTTCCTACTCAGGGGACTTTC | 1273 | ELPTQGTFSNVSTNVS | 1274 |
| epitope | TCAAACGTTAGCACAAACGTAAGT | | | |
| CD8 | CCCGCCCCAAGACCCCCCACACC | 1275 | PAPRPPTPAPTIASQPLSLRPEAC | 1276 |
| stalk | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1277 | IYIWAPLAGTCGVLLLSLVITLYCN | 1278 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1279 | VD | 1280 |
| CD3ζ | AGAGTGAAGTTCAGCAGGAGCGC | 1281 | RVKFSRSADAPAYQQGQNQLYN | 1282 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| P2A | GCAACGAATTTTTCCCTGCTGAAA | 1283 | ATNFSLLKQAGDVEENPGP | 1284 |
| | CAGGCAGGGGACGTAGAGGAAAA | | | |
| | TCCTGGTCCT | | | |
| MyD88 | atggctgcaggaggtcccggcgcggggtctgcg | 1285 | MAAGGPGAGSAAPVSSTSSLPLA | 1286 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQAD | |
| | gttcttgaacgtgcggacacaggtggcggccga | | PTGRLLDAWQGRPGASVGRLLD | |
| | ctggaccgcgctggcggaggagatggactttga | | LLTKLGRDDVLLELGPSIEEDCQK | |
| | gtacttggagatccggcaactggagacacaagc | | YILKQQQEEAEKPLQVAAVDSSV | |
| | ggaccccactggcaggctgctggacgcctggc | | PRTAELAGITTLDDPLGHMPERF | |
| | agggacgccctggcgcctctgtaggccgactgc | | DAFICYCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgcta | | | |
| | ttgccccagcgacatc | | | |
| Linker | gtcgag | 1287 | VE | 1288 |
| CD40 | aaaaaggtggccaagaagccaaccaataagg | 1289 | KKVAKKPTNKAPHPKQEPQEINF | 1290 |
| | ccccccaccccaagcaggagccccaggagat | | PDDLPGSNTAAPVQETLHGCQP | |
| | caattttcccgacgatcttcctggctccaacactgc | | VTQEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| STOP | TGA | 1291 | stop | |

APPENDIX 9
pBP1321—pSFG-FKBP.FRB.ΔC9.T2A-αPSCA.Q.CD8stm.ζ.P2A-MC.FKBP_{V}.FKBP

| | | SEQ | | SEQ |
|---|---|---|---|---|
| | | ID | | ID |
| Fragment | Nucleotide | NO: | Peptide | NO: |
| Leader | ATGCtcgagcaattg | 1292 | MLEQL | 1293 |
| peptide | | | | |
| FKBP″wt | GGcGTGCAaGTGGAaACTATaAGC | 1294 | GVQVETISPGDGRTFPKRGQTCV | 1295 |
| | CCgGGAGAcGGCcGcACATTtCCC | | VHYTGMLEDGKKFDSSRDRNKP | |
| | AAgAGAGGcCAGACcTGCGTgGTG | | FKFMLGKQEVIRGWEEGVAQMS | |
| | CAcTATACaGGAATGCTGGAgGAC | | VGQRAKLTISPDYAYGATGHPGII | |
| | GGgAAGAAaTTCGAtAGCtcCCGGG | | PPHATLVFDVELLKLE | |
| | AtCGAAAtAAGCCtTTCAAaTTCATG | | | |
| | CTGGGcAAGCAaGAAGTcATCaGa | | | |
| | GGCTGGGAaGAAGGcGTCGCcCA | | | |
| | GATGTCcGTGGGtCAGcGcGCCAA | | | |
| | gCTGACaATTAGtCCAGAtTACGCc | | | |
| | TATGGcGCAACaGGCCAtCCCGGc | | | |
| | ATCATcCCCCCaCATGCcACACTc | | | |
| | GTCTTtGATGTcGAGCTcCTGAAaC | | | |
| | TGGAg | | | |
| Linker | GGCGGGcaattg | 1296 | ggql | 1297 |
| FRB | gaaatgTGGCATGAAGGGTTGGAAG | 1298 | EMWHEGLEEASRLYFGERNVKG | 1299 |
| | AAGCTTCAAGGCTGTACTTCGGA | | MFEVLEPLHAMMERGPQTLKETS | |
| | GAGAGGAACGTGAAGGGCATGTT | | FNQAYGRDLMEAQEWCRKYMK | |
| | TGAGGTTCTTGAACCTCTGCACG | | SGNVKDLTQAVVDLYYHVFRRISK | |
| | CCATGATGGAACGGGGACCGCAG | | | |
| | ACACTGAAAGAAACCTCTTTTAAT | | | |
| | CAGGCCTACGGCAGAGACCTGAT | | | |
| | GGAGGCCCAAGAATGGTGTAGAA | | | |
| | AGTATATGAAATCCGGTAACGTGA | | | |
| | AAGACCTGactCAGGCCTGGGACC | | | |
| | TTTATTACCATGTGTTCAGGCGGA | | | |
| | TCAGTAAG | | | |
| Linker | TCAGGCGGTGGCTCAGGTccatgg | 1300 | SGGGSGPW | 1301 |
| Δcaspase9 | GGATTTGGTGATGTCGGTGCTCT | 1302 | GFGDVGALESLRGNADLAYILSM | 1303 |
| | TGAGAGTTTGAGGGGAAATGCAG | | EPCGHCLIINNVNFCRESGLRTRT | |
| | ATTTGGCTTACATCCTGAGCATGG | | GSNIDCEKLRRRFSSLHFMVEVK | |
| | AGCCCTGTGGCCACTGCCTCATT | | GDLTAKKMVLALLELARQDHGAL | |
| | ATCAACAATGTGAACTTCTGCCGT | | DCCVVVILSHGCQASHLQFPGAV | |
| | GAGTCCGGGCTCCGCACCCGCA | | YGTDGCPVSVEKIVNIFNGTSCPS | |
| | CTGGCTCCAACATCGACTGTGAG | | LGGKPKLFFIQACGGEQKDHGFE | |
| | AAGTTGCGGCGTCGCTTCTCCTC | | VASTSPEDESPGSNPEPDATPFQ | |
| | GCTGCATTTCATGGTGGAGGTGA | | EGLRTFDQLDAISSLPTPSDIFVS | |
| | AGGGCGACCTGACTGCCAAGAAA | | YSTFPGFVSVVRDPKSGSVVYVET | |
| | ATGGTGCTGGCTTTGCTGGAGCT | | LDDIFEQWAHSEDLQSLLLRVAN | |
| | GGCGCgGCAGGACCACGGTGCTC | | AVSVKGIYKQMPGCFNFLRKKLF | |
| | TGGACTGCTGCGTGGTGGTCATT | | FKTSASRA | |
| | CTCTCTCACGGCTGTCAGGCCAG | | | |
| | CCACCTGCAGTTCCCAGGGGCTG | | | |
| | TCTACGGCACAGATGGATGCCCT | | | |
| | GTGTCGGTCGAGAAGATTGTGAA | | | |
| | CATCTTCAATGGGACCAGCTGCC | | | |
| | CCAGCCTGGGAGGGAAGCCCAA | | | |
| | GCTCTTTTTCATCCAGGCCTGTGG | | | |
| | TGGGGAGCAGAAAGAtCATGGGT | | | |
| | TTGAGGTGGCCTCCACTTCCCCT | | | |
| | GAAGACGAGTCCCCTGGCAGTAA | | | |
| | CCCCGAGCCAGATGCCACCCCGT | | | |
| | TCCAGGAAGGTTTGAGGACCTTC | | | |
| | GACCAGCTGGACGCCATATCTAG | | | |
| | TTTGCCCACACCCAGTGACATCTT | | | |
| | TGTGTCCTACTCTACTTTCCCAGG | | | |
| | TTTTGTTTCCTGGAGGGACCCCAA | | | |
| | GAGTGGCTCCTGGTACGTTGAGA | | | |
| | CCCTGGACGACATCTTTGAGCAG | | | |
| | TGGGCTCACTCTGAAGACCTGCA | | | |
| | GTCCCTCCTGCTTAGGGTCGCTA | | | |
| | ATGCTGTTTCGGTGAAAGGGATTT | | | |
| | ATAAACAGATGCCTGGTTGCTTTA | | | |
| | ATTTCCTCCGGAAAAAACTTTTCT | | | |
| | TTAAAACATCAGCTAGCAGAGCC | | | |
| Linker | ggatctggaccgcGG | 1304 | GSGPR | 1305 |
| T2A | GAAGGCCGAGGGAGCCTGCTGA | 1306 | EGRGSLLTCGDVEENPGP | 1307 |
| | CATGTGGCGATGTGGAGGAAAAC | | | |
| | CCAGGACCA | | | |
| Linker | CCATGG | 1308 | PW | 1309 |
| Signal | ATGGAGTTTGGACTTTCTTGGTTG | 1310 | MEFGLSVVLFLVAILKGVQCSR | 1311 |
| Peptide | TTTTTGGTGGCAATTCTGAAGGGT | | | |
| | GTCCAGTGTAGCAGG | | | |
| PSCA(A11) | GACATCCAACTGACGCAAAGCCC | 1312 | DIQLTQSPSTLSASMGDRVTITCS | 1313 |
| VL | ATCTACACTCAGCGCTAGCATGG | | ASSSVRFIHVVYQQKPGKAPKRLI | |
| | GGGACAGGGTCACAATCACGTGC | | YDTSKLASGVPSRFSGSGSGTDF | |
| | TCTGCCTCAAGTTCCGTTAGGTTT | | TLTISSLQPEDFATYYCQQWGSS | |
| | ATCCATTGGTATCAGCAGAAACCT | | PFTFGQGTKVEIK | |
| | GGAAAGGCCCCAAAAAGACTGAT | | | |
| | CTATGATACCAGCAAGCTGGCTT | | | |
| | CCGGAGTGCCCTCAAGGTTCTCA | | | |
| | GGATCTGGCAGTGGGACCGATTT | | | |
| | CACCCTGACAATTAGCAGCCTTCA | | | |
| | GCCAGAGGATTTCGCAACCTATTA | | | |
| | CTGTCAGCAATGGGGGTCCAGCC | | | |
| | CATTCACTTTCGGCCAAGGAACAA | | | |
| | AGGTGGAGATAAAA | | | |
| Flex | GGCGGAGGAAGCGGAGGTGGGG | 1314 | gggsgggg | 1315 |
| | GC | | | |
| PSCA(A11) | GAGGTGCAGCTCGTGGAGTATGG | 1316 | EVQLVEYGGGLVQPGGSLRLSC | 1317 |
| VH | CGGGGGCCTGGTGCAGCCTGGG | | AASGFNIKDYYIHVVVRQAPGKGL | |
| | GGTAGTCTGAGGCTCTCCTGCGC | | EVVVAWIDPENGDTEFVPKFQGR | |
| | TGCCTCTGGCTTTAACATTAAAGA | | ATMSADTSKNTAYLQMNSLRAED | |
| | CTACTACATACATTGGGTGCGGC | | TAVYYCKTGGFWGQGTLVTVSS | |
| | AGGCCCCAGGCAAAGGGCTCGAA | | | |
| | TGGGTGGCCTGGATTGACCCTGA | | | |
| | GAATGGTGACACTGAGTTTGTCC | | | |
| | CCAAGTTTCAGGGCAGAGCCACC | | | |
| | ATGAGCGCTGACACAAGCAAAAA | | | |
| | CACTGCTTATCTCCAAATGAATAG | | | |
| | CCTGCGAGCTGAAGATACAGCAG | | | |
| | TCTATTACTGCAAGACGGGAGGA | | | |
| | TTCTGGGGCCAGGGAACTCTGGT | | | |
| | GACAGTTAGTTCC | | | |
| Linker | GGATCC | 1318 | gs | 1319 |
| CD34 | GAACTTCCTACTCAGGGGACTTTC | 1320 | ELPTQGTFSNVSTNVS | 1321 |
| epitope | TCAAACGTTAGCACAAACGTAAGT | | | |
| CD8 stalk | CCCGCCCCAAGACCCCCCACACC | 1322 | PAPRPPTPAPTIASQPLSLRPEAC | 1323 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1324 | IYIWAPLAGTCGVLLLSLVITLYCN | 1325 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1326 | VD | 1327 |
| CD3ζ | AGAGTGAAGTTCAGCAGGAGCGC | 1328 | RVKFSRSADAPAYQQGQNQLYN | 1329 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | gGAACGCGTGGATCGGGA | 1330 | GTRGSG | 1331 |
| P2A | GCTACTAACTTCAGCCTGCTGAA | 1332 | ATNFSLLKQAGDVEENPGP | 1333 |
| | GCAGGCTGGAGACGTGGAGGAG | | | |
| | AACcccgggcct | | | |
| MyD88 | atggctgcaggaggtcccggcgcggggtctgcg | 1334 | MAAGGPGAGSAAPVSSTSSLPLA | 1335 |
| | gccccggtctcctccacatcctcccttcccctggct | | ALNMRVRRRLSLFLNVRTQVAAD | |
| | gctctcaacatgcgagtgcggcgccgcctgtctct | | WTALAEEMDFEYLEIRQLETQAD | |
| | gttcttgaacgtgcggacacaggtggcggccga | | PTGRLLDAWQGRPGASVGRLLD | |
| | ctggaccgcgctggcggaggagatggactttga | | LLTKLGRDDVLLELGPSIEEDCQK | |
| | gtacttggagatccggcaactggagacacaagc | | YILKQQQEEAEKPLQVAAVDSSV | |
| | ggaccccactggcaggctgctggacgcctggc | | PRTAELAGITTLDDPLGHMPERF | |
| | agggacgccctggcgcctctgtaggccgactgc | | DAFICYCPSDI | |
| | tcgatctgcttaccaagctgggccgcgacgacgt | | | |
| | gctgctggagctgggacccagcattgaggagg | | | |
| | attgccaaaagtatatcttgaagcagcagcagg | | | |
| | aggaggctgagaagcctttacaggtggccgctg | | | |
| | tagacagcagtgtcccacggacagcagagctg | | | |
| | gcgggcatcaccacacttgatgaccccctgggg | | | |
| | catatgcctgagcgtttcgatgccttcatctgct | | | |
| | attgccccagcgacatc | | | |
| Linker | gtcgag | 1336 | VE | 1337 |
| CD40 | aaaaaggtggccaagaagccaaccaataagg | 1338 | KKVAKKPTNKAPHPKQEPQEINF | 1339 |
| | ccccccaccccaagcaggagccccaggagat | | PDDLPGSNTAAPVQETLHGCQP | |
| | caattttcccgacgatcttcctggctccaacactgc | | VTQEDGKESRISVQERQ | |
| | tgctccagtgcaggagactttacatggatgccaa | | | |
| | ccggtcacccaggaggatggcaaagagagtcg | | | |
| | catctcagtgcaggagagacag | | | |
| Linker | gtcgag | 1340 | VE | 1341 |
| FKBP_{V}_{^{′}} | GGcGTcCAaGTcGAaACcATtagtCCc | 1342 | GVQVETISPGDGRTFPKRGQTCV | 1343 |
| | GGcGAtGGcaGaACaTTtCCtAAaaG | | VHYTGMLEDGKKVDSSRDRNKP | |
| | gGGaCAaACaTGtGTcGTcCAtTAtA | | FKFMLGKQEVIRGWEEGVAQMS | |
| | CaGGcATGtTgGAgGAcGGcAAaAA | | VGQRAKLTISPDYAYGATGHPGII | |
| | gGTgGAcagtagtaGaGAtcGcAAtAAa | | PPHATLVFDVELLKLE | |
| | CCtTTcAAaTTcATGtTgGGaAAaCAa | | | |
| | GAaGTcATtaGgGGaTGGGAgGAgG | | | |
| | GcGTgGCtCAaATGtccGTcGGcCAa | | | |
| | cGcGCtAAgCTcACcATcagcCCcGAc | | | |
| | TAcGCaTAcGGcGCtACcGGaCAtCC | | | |
| | cGGaATtATtCCcCCtCAcGCtACctTg | | | |
| | GTgTTtGAcGTcGAaCTgtTgAAgCTc | | | |
| | GAa | | | |
| Linker | gtcgag | 1344 | VE | 1345 |
| FKBP wt | ggagtgcaggtggagactatctccccaggagac | 1346 | GVQVETISPGDGRTFPKRGQTCV | 1347 |
| | gggcgcaccttccccaagcgcggccagacctg | | VHYTGMLEDGKKFDSSRDRNKP | |
| | cgtggtgcactacaccgggatgcttgaagatgg | | FKFMLGKQEVIRGWEEGVAQMS | |
| | aaagaaaTttgattcctcccgggacagaaaca | | VGQRAKLTISPDYAYGATGHPGII | |
| | agccctttaagtttatgctaggcaagcaggaggt | | PPHATLVFDVELLKLE | |
| | gatccgaggctgggaagaaggggttgcccaga | | | |
| | tgagtgtgggtcagagagccaaactgactatatc | | | |
| | tccagattatgcctatggtgccactgggcaccca | | | |
| | ggcatcatcccaccacatgccactctcgtcttcga | | | |
| | tgtggagcttctaaaactggaa | | | |
| STOP | TGA | 1348 | stop | |

APPENDIX 10
pBP1151—pSFG—MC-T2A-αCD19.Q.CD8stm.ζ

| | | SEQ | | SEQ |
|---|---|---|---|---|
| | | ID | | ID |
| Fragment | Nucleotide | NO: | Peptide | NO: |
| MyD88 | ATGGCTGCAGGAGGTCCCGGCG | 1349 | MAAGGPGAGSAAPVSSTSSLPLA | 1350 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YlLKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1351 | VE | 1352 |
| CD40 | AAAAAGGTGGCCAAGAAGCCAAC | 1353 | KKVAKKPTNKAPHPKQEPQEINF | 1354 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GGATCTGGACCGCGG | 1355 | GSGPR | 1356 |
| T2A | GAAGGCCGAGGGAGCCTGCTGA | 1357 | EGRGSLLTCGDVEENPGP | 1358 |
| | CATGTGGCGATGTGGAGGAAAAC | | | |
| | CCAGGACCA | | | |
| Linker | CCACGG | 1359 | PR | 1360 |
| Signal | ATGGAGTTTGGACTTTCTTGGTTG | 1361 | MEFGLSVVLFLVAILKGVQCSR | 1362 |
| Peptide | TTTTTGGTGGCAATTCTGAAGGGT | | | |
| | GTCCAGTGTAGCAGG | | | |
| FMC63 VL | GACATCCAGATGACACAGACTAC | 1363 | DIQMTQTTSSLSASLGDRVTISCR | 1364 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1365 | GGGSGGGG | 1366 |
| | GC | | | |
| FMC63 VH | GAGGTGAAACTGCAGGAGTCAGG | 1367 | EVKLQESGPGLVAPSQSLSVTCT | 1368 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1369 | GS | 1370 |
| CD34 | GAACTTCCTACTCAGGGGACTTTC | 1371 | ELPTQGTFSNVSTNVS | 1372 |
| epitope | TCAAACGTTAGCACAAACGTAAGT | | | |
| CD8 | CCCGCCCCAAGACCCCCCACACC | 1373 | PAPRPPTPAPTIASQPLSLRPEAC | 1374 |
| stalk | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1375 | IYIWAPLAGTCGVLLLSLVITLYCN | 1376 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1377 | VD | 1378 |
| CD3ζ | AGAGTGAAGTTCAGCAGGAGCGC | 1379 | RVKFSRSADAPAYQQGQNQLYN | 1380 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |

APPENDIX 11
pBP1152—pSFG—MC-T2A-αCD19.Q.CD8stm.ζ

| | | SEQ | | SEQ |
|---|---|---|---|---|
| | | ID | | ID |
| Fragment | Nucleotide | NO: | Peptide | NO: |
| Myristoylation | ATGGGGAGTAGCAAGAGCAAG | 1381 | MGSSKSKPKDPSQR | 1382 |
| Targeting | CCTAAGGACCCCAGCCAGCGC | | | |
| sequence | | | | |
| MyD88 | ATGGCTGCAGGAGGTCCCGGC | 1383 | MAAGGPGAGSAAPVSSTSSLPLA | 1384 |
| | GCGGGGTCTGCGGCCCCGGT | | ALNMRVRRRLSLFLNVRTQVAAD | |
| | CTCCTCCACATCCTCCCTTCCC | | WTALAEEMDFEYLEIRQLETQAD | |
| | CTGGCTGCTCTCAACATGCGA | | PTGRLLDAWQGRPGASVGRLLD | |
| | GTGCGGCGCCGCCTGTCTCTG | | LLTKLGRDDVLLELGPSIEEDCQK | |
| | TTCTTGAACGTGCGGACACAG | | YILKQQQEEAEKPLQVAAVDSSV | |
| | GTGGCGGCCGACTGGACCGC | | PRTAELAGITTLDDPLGHMPERF | |
| | GCTGGCGGAGGAGATGGACTT | | DAFICYCPSDI | |
| | TGAGTACTTGGAGATCCGGCA | | | |
| | ACTGGAGACACAAGCGGACCC | | | |
| | CACTGGCAGGCTGCTGGACGC | | | |
| | CTGGCAGGGACGCCCTGGCG | | | |
| | CCTCTGTAGGCCGACTGCTCG | | | |
| | ATCTGCTTACCAAGCTGGGCC | | | |
| | GCGACGACGTGCTGCTGGAGC | | | |
| | TGGGACCCAGCATTGAGGAGG | | | |
| | ATTGCCAAAAGTATATCTTGAA | | | |
| | GCAGCAGCAGGAGGAGGCTG | | | |
| | AGAAGCCTTTACAGGTGGCCG | | | |
| | CTGTAGACAGCAGTGTCCCAC | | | |
| | GGACAGCAGAGCTGGCGGGC | | | |
| | ATCACCACACTTGATGACCCC | | | |
| | CTGGGGCATATGCCTGAGCGT | | | |
| | TTCGATGCCTTCATCTGCTATT | | | |
| | GCCCCAGCGACATC | | | |
| Linker | GTCGAG | 1385 | VE | 1386 |
| CD40 | AAAAAGGTGGCCAAGAAGCCA | 1387 | KKVAKKPTNKAPHPKQEPQEINF | 1388 |
| | ACCAATAAGGCCCCCCACCCC | | PDDLPGSNTAAPVQETLHGCQP | |
| | AAGCAGGAGCCCCAGGAGATC | | VTQEDGKESRISVQERQ | |
| | AATTTTCCCGACGATCTTCCTG | | | |
| | GCTCCAACACTGCTGCTCCAG | | | |
| | TGCAGGAGACTTTACATGGAT | | | |
| | GCCAACCGGTCACCCAGGAGG | | | |
| | ATGGCAAAGAGAGTCGCATCT | | | |
| | CAGTGCAGGAGAGACAG | | | |
| Linker | GGATCTGGACCGCGG | 1389 | GSGPR | 1390 |
| T2A | GAAGGCCGAGGGAGCCTGCT | 1391 | EGRGSLLTCGDVEENPGP | 1392 |
| | GACATGTGGCGATGTGGAGGA | | | |
| | AAACCCAGGACCA | | | |
| Linker | CCACGG | 1393 | PR | 1394 |
| Signal Peptide | ATGGAGTTTGGACTTTCTTGGT | 1395 | MEFGLSVVLFLVAILKGVQCSR | 1396 |
| | TGTTTTTGGTGGCAATTCTGAA | | | |
| | GGGTGTCCAGTGTAGCAGG | | | |
| FMC63 VL | GACATCCAGATGACACAGACT | 1397 | DIQMTQTTSSLSASLGDRVTISCR | 1398 |
| | ACATCCTCCCTGTCTGCCTCTC | | ASQDISKYLNVVYQQKPDGTVKLL | |
| | TGGGAGACAGAGTCACCATCA | | IYHTSRLHSGVPSRFSGSGSGTD | |
| | GTTGCAGGGCAAGTCAGGACA | | YSLTISNLEQEDIATYFCQQGNTL | |
| | TTAGTAAATATTTAAATTGGTAT | | PYTFGGGTKLEIT | |
| | CAGCAGAAACCAGATGGAACT | | | |
| | GTTAAACTCCTGATCTACCATA | | | |
| | CATCAAGATTACACTCAGGAGT | | | |
| | CCCATCAAGGTTCAGTGGCAG | | | |
| | TGGGTCTGGAACAGATTATTCT | | | |
| | CTCACCATTAGCAACCTGGAG | | | |
| | CAAGAAGATATTGCCACTTACT | | | |
| | TTTGCCAACAGGGTAATACGCT | | | |
| | TCCGTACACGTTCGGAGGGGG | | | |
| | GACTAAGTTGGAAATAACA | | | |
| Flex | GGCGGAGGAAGCGGAGGTGG | 1399 | GGGSGGGG | 1400 |
| | GGGC | | | |
| FMC63 VH | GAGGTGAAACTGCAGGAGTCA | 1401 | EVKLQESGPGLVAPSQSLSVTCT | 1402 |
| | GGACCTGGCCTGGTGGCGCC | | VSGVSLPDYGVSWIRQPPRKGLE | |
| | CTCACAGAGCCTGTCCGTCAC | | WLGVIWGSETTYYNSALKSRLTII | |
| | ATGCACTGTCTCAGGGGTCTC | | KDNSKSQVFLKMNSLQTDDTAIY | |
| | ATTACCCGACTATGGTGTAAGC | | YCAKHYYYGGSYAMDYWGQGT | |
| | TGGATTCGCCAGCCTCCACGA | | SVTVSS | |
| | AAGGGTCTGGAGTGGCTGGGA | | | |
| | GTAATATGGGGTAGTGAAACC | | | |
| | ACATACTATAATTCAGCTCTCA | | | |
| | AATCCAGACTGACCATCATCAA | | | |
| | GGACAACTCCAAGAGCCAAGT | | | |
| | TTTCTTAAAAATGAACAGTCTG | | | |
| | CAAACTGATGACACAGCCATTT | | | |
| | ACTACTGTGCCAAACATTATTA | | | |
| | CTACGGTGGTAGCTATGCTAT | | | |
| | GGACTACTGGGGTCAAGGAAC | | | |
| | CTCAGTCACCGTCTCCTCA | | | |
| Linker | GGATCC | 1403 | GS | 1404 |
| CD34 epitope | GAACTTCCTACTCAGGGGACTT | 1405 | ELPTQGTFSNVSTNVS | 1406 |
| | TCTCAAACGTTAGCACAAACGT | | | |
| | AAGT | | | |
| CD8 stalk | CCCGCCCCAAGACCCCCCACA | 1407 | PAPRPPTPAPTIASQPLSLRPEAC | 1408 |
| | CCTGCGCCGACCATTGCTTCT | | RPAAGGAVHTRGLDFACD | |
| | CAACCCCTGAGTTTGAGACCC | | | |
| | GAGGCCTGCCGGCCAGCTGC | | | |
| | CGGCGGGGCCGTGCATACAA | | | |
| | GAGGACTCGATTTCGCTTGCG | | | |
| | AC | | | |
| CD8 | ATCTATATCTGGGCACCTCTCG | 1409 | IYIWAPLAGTCGVLLLSLVITLYC | 1410 |
| trans- | CTGGCACCTGTGGAGTCCTTC | | NHRNRRRVCKCPR | |
| membrane | TGCTCAGCCTGGTTATTACTCT | | | |
| | GTACTGTAATCACCGGAATCG | | | |
| | CCGCCGCGTTTGTAAGTGTCC | | | |
| | CAGG | | | |
| Linker | GTCGAC | 1411 | VD | 1412 |
| CD3ζ | AGAGTGAAGTTCAGCAGGAGC | 1413 | RVKFSRSADAPAYQQGQNQLYN | 1414 |
| | GCAGACGCCCCCGCGTACCAG | | ELNLGRREEYDVLDKRRGRDPE | |
| | CAGGGCCAGAACCAGCTCTAT | | MGGKPRRKNPQEGLYNELQKDK | |
| | AACGAGCTCAATCTAGGACGA | | MAEAYSEIGMKGERRRGKGHDG | |
| | AGAGAGGAGTACGATGTTTTG | | LYQGLSTATKDTYDALHMQALPP | |
| | GACAAGAGACGTGGCCGGGA | | R | |
| | CCCTGAGATGGGGGGAAAGCC | | | |
| | GAGAAGGAAGAACCCTCAGGA | | | |
| | AGGCCTGTACAATGAACTGCA | | | |
| | GAAAGATAAGATGGCGGAGGC | | | |
| | CTACAGTGAGATTGGGATGAA | | | |
| | AGGCGAGCGCCGGAGGGGCA | | | |
| | AGGGGCACGATGGCCTTTACC | | | |
| | AGGGTCTCAGTACAGCCACCA | | | |
| | AGGACACCTACGACGCCCTTC | | | |
| | ACATGCAAGCTCTTCCACCTCG | | | |
| | T | | | |

APPENDIX 12
pBP1414—pSFG-αCD19.Q.CD8stm.ζ-P2A-MC

| | | SEQ | | SEQ |
|---|---|---|---|---|
| | | ID | | ID |
| Fragment | Nucleotide | NO: | Peptide | NO: |
| Signal | ATGGAGTTTGGACTTTCTTGGTTG | 1415 | MEFGLSVVLFLVAILKGVQCSR | 1416 |
| Peptide | TTTTTGGTGGCAATTCTGAAGGGT | | | |
| | GTCCAGTGTAGCAGG | | | |
| FMC63 VL | GACATCCAGATGACACAGACTAC | 1417 | DIQMTQTTSSLSASLGDRVTISCR | 1418 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1419 | GGGSGGGG | 1420 |
| | GC | | | |
| FMC63 VH | GAGGTGAAACTGCAGGAGTCAGG | 1421 | EVKLQESGPGLVAPSQSLSVTCT | 1422 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1423 | GS | 1424 |
| CD34 | GAACTTCCTACTCAGGGGACTTTC | 1425 | ELPTQGTFSNVSTNVS | 1426 |
| epitope | TCAAACGTTAGCACAAACGTAAGT | | | |
| CD8 stalk | CCCGCCCCAAGACCCCCCACACC | 1427 | PAPRPPTPAPTIASQPLSLRPEAC | 1428 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1429 | IYIWAPLAGTCGVLLLSLVITLYCN | 1430 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1431 | VD | 1432 |
| CD3ζ | AGAGTGAAGTTCAGCAGGAGCGC | 1433 | RVKFSRSADAPAYQQGQNQLYN | 1434 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| P2A | GCTACTAACTTCAGCCTGCTGAA | 1435 | ATNFSLLKQAGDVEENPGP | 1436 |
| | GCAGGCTGGAGACGTGGAGGAG | | | |
| | AACCCCGGGCCT | | | |
| MyD88 | ATGGCTGCAGGAGGTCCCGGCG | 1437 | MAAGGPGAGSAAPVSSTSSLPLA | 1438 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1439 | VE | 1440 |
| CD40 | AAAAAGGTGGCCAAGAAGCCAAC | 1441 | KKVAKKPTNKAPHPKQEPQEINF | 1442 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |

APPENDIX 13
pBP1414—pSFG-αCD19.Q.CD8stm.ζ-P2A-MC

| | | SEQ | | SEQ |
|---|---|---|---|---|
| | | ID | | ID |
| Fragment | Nucleotide | NO: | Peptide | NO: |
| Signal | ATGGAGTTTGGACTTTCTTGGTTG | 1443 | MEFGLSVVLFLVAILKGVQCSR | 1444 |
| Peptide | TTTTTGGTGGCAATTCTGAAGGGT | | | |
| | GTCCAGTGTAGCAGG | | | |
| FMC63 VL | GACATCCAGATGACACAGACTAC | 1445 | DIQMTQTTSSLSASLGDRVTISCR | 1446 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1447 | GGGSGGGG | 1448 |
| | GC | | | |
| FMC63 VH | GAGGTGAAACTGCAGGAGTCAGG | 1449 | EVKLQESGPGLVAPSQSLSVTCT | 1450 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1451 | GS | 1452 |
| CD34 | GAACTTCCTACTCAGGGGACTTTC | 1453 | ELPTQGTFSNVSTNVS | 1454 |
| epitope | TCAAACGTTAGCACAAACGTAAGT | | | |
| CD8 | CCCGCCCCAAGACCCCCCACACC | 1455 | PAPRPPTPAPTIASQPLSLRPEAC | 1456 |
| stalk | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1457 | IYIWAPLAGTCGVLLLSLVITLYCN | 1458 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1459 | VD | 1460 |
| CD3ζ | AGAGTGAAGTTCAGCAGGAGCGC | 1461 | RVKFSRSADAPAYQQGQNQLYN | 1462 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| P2A | GCTACTAACTTCAGCCTGCTGAA | 1463 | ATNFSLLKQAGDVEENPGP | 1464 |
| | GCAGGCTGGAGACGTGGAGGAG | | | |
| | AACCCCGGGCCT | | | |
| MyD88 | ATGGCTGCAGGAGGTCCCGGCG | 1465 | MAAGGPGAGSAAPVSSTSSLPLA | 1466 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1467 | VE | 1468 |
| CD40 | AAAAAGGTGGCCAAGAAGCCAAC | 1469 | KKVAKKPTNKAPHPKQEPQEINF | 1470 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |

APPENDIX 14
pBP1433—pSFG-Fv-Fv-MC-T2A-αCD19.Q.CD8stm.ζ

| | | SEQ | | SEQ |
|---|---|---|---|---|
| | | ID | | ID |
| Fragment | Nucleotide | NO: | Peptide | NO: |
| FKBP_{V}_{^{′}} | GGCGTCCAAGTCGAAACCATTAG | 1471 | GVQVETISPGDGRTFPKRGQTCV | 1472 |
| | TCCCGGCGATGGCAGAACATTTC | | VHYTGMLEDGKKVDSSRDRNKP | |
| | CTAAAAGGGGACAAACATGTGTC | | FKFMLGKQEVIRGWEEGVAQMS | |
| | GTCCATTATACAGGCATGTTGGA | | VGQRAKLTISPDYAYGATGHPGII | |
| | GGACGGCAAAAAGGTGGACAGTA | | PPHATLVFDVELLKLE | |
| | GTAGAGATCGCAATAAACCTTTCA | | | |
| | AATTCATGTTGGGAAAACAAGAAG | | | |
| | TCATTAGGGGATGGGAGGAGGGC | | | |
| | GTGGCTCAAATGTCCGTCGGCCA | | | |
| | ACGCGCTAAGCTCACCATCAGCC | | | |
| | CCGACTACGCATACGGCGCTACC | | | |
| | GGACATCCCGGAATTATTCCCCC | | | |
| | TCACGCTACCTTGGTGTTTGACGT | | | |
| | CGAACTGTTGAAGCTCGAA | | | |
| Linker | GTCGAG | 1473 | VE | 1474 |
| FKBP_{V} | GGAGTGCAGGTGGAGACTATCTC | 1475 | GVQVETISPGDGRTFPKRGQTCV | 1476 |
| | CCCAGGAGACGGGCGCACCTTCC | | VHYTGMLEDGKKVDSSRDRNKP | |
| | CCAAGCGCGGCCAGACCTGCGT | | FKFMLGKQEVIRGWEEGVAQMS | |
| | GGTGCACTACACCGGGATGCTTG | | VGQRAKLTISPDYAYGATGHPGII | |
| | AAGATGGAAAGAAAGTTGATTCCT | | PPHATLVFDVELLKLE | |
| | CCCGGGACAGAAACAAGCCCTTT | | | |
| | AAGTTTATGCTAGGCAAGCAGGA | | | |
| | GGTGATCCGAGGCTGGGAAGAAG | | | |
| | GGGTTGCCCAGATGAGTGTGGGT | | | |
| | CAGAGAGCCAAACTGACTATATCT | | | |
| | CCAGATTATGCCTATGGTGCCACT | | | |
| | GGGCACCCAGGCATCATCCCACC | | | |
| | ACATGCCACTCTCGTCTTCGATGT | | | |
| | GGAGCTTCTAAAACTGGAA | | | |
| MyD88 | ATGGCTGCAGGAGGTCCCGGCG | 1477 | MAAGGPGAGSAAPVSSTSSLPLA | 1478 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1479 | VE | 1480 |
| CD40 | AAAAAGGTGGCCAAGAAGCCAAC | 1481 | KKVAKKPTNKAPHPKQEPQEINF | 1482 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GGATCTGGACCGCGG | 1483 | GSGPR | 1484 |
| T2A | GAAGGCCGAGGGAGCCTGCTGA | 1485 | EGRGSLLTCGDVEENPGP | 1486 |
| | CATGTGGCGATGTGGAGGAAAAC | | | |
| | CCAGGACCA | | | |
| Linker | CCACGG | 1487 | PR | 1488 |
| Signal | ATGGAGTTTGGACTTTCTTGGTTG | 1489 | MEFGLSVVLFLVAILKGVQCSR | 1490 |
| Peptide | TTTTTGGTGGCAATTCTGAAGGGT | | | |
| | GTCCAGTGTAGCAGG | | | |
| FMC63 VL | GACATCCAGATGACACAGACTAC | 1491 | DIQMTQTTSSLSASLGDRVTISCR | 1492 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1493 | GGGSGGGG | 1494 |
| | GC | | | |
| FMC63 VH | GAGGTGAAACTGCAGGAGTCAGG | 1495 | EVKLQESGPGLVAPSQSLSVTCT | 1496 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1497 | GS | 1498 |
| CD34 | GAACTTCCTACTCAGGGGACTTTC | 1499 | ELPTQGTFSNVSTNVS | 1500 |
| epitope | TCAAACGTTAGCACAAACGTAAGT | | | |
| CD8 stalk | CCCGCCCCAAGACCCCCCACACC | 1501 | PAPRPPTPAPTIASQPLSLRPEAC | 1502 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1503 | IYIWAPLAGTCGVLLLSLVITLYCN | 1504 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1505 | VD | 1506 |
| CD3ζ | AGAGTGAAGTTCAGCAGGAGCGC | 1507 | RVKFSRSADAPAYQQGQNQLYN | 1508 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |

APPENDIX 15
pBP1439—pSFG—MC.FKBPᵥ-T2A-αCD19.Q.CD8stm.ζ

| | | SEQ | | SEQ |
|---|---|---|---|---|
| | | ID | | ID |
| Fragment | Nucleotide | NO: | Peptide | NO: |
| MyD88 | ATGGCTGCAGGAGGTCCCGGCG | 1509 | MAAGGPGAGSAAPVSSTSSLPLA | 1510 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YlLKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1511 | VE | 1512 |
| CD40 | AAAAAGGTGGCCAAGAAGCCAAC | 1513 | KKVAKKPTNKAPHPKQEPQEINF | 1514 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 1515 | VE | 1516 |
| FKBPv | GGAGTGCAGGTGGAGACTATTAG | 1517 | GVQVETISPGDGRTFPKRGQTCV | 1518 |
| | CCCCGGAGATGGCAGAACATTCC | | VHYTGMLEDGKKVDSSRDRNKP | |
| | CCAAAAGAGGACAGACTTGCGTC | | FKFMLGKQEVIRGWEEGVAQMS | |
| | GTGCATTATACTGGAATGCTGGAA | | VGQRAKLTISPDYAYGATGHPGII | |
| | GACGGCAAGAAGGTGGACAGCA | | PPHATLVFDVELLKLE | |
| | GCCGGGACCGAAACAAGCCCTTC | | | |
| | AAGTTCATGCTGGGGAAGCAGGA | | | |
| | AGTGATCCGGGGCTGGGAGGAA | | | |
| | GGAGTCGCACAGATGTCAGTGGG | | | |
| | ACAGAGGGCCAAACTGACTATTA | | | |
| | GCCCAGACTACGCTTATGGAGCA | | | |
| | ACCGGCCACCCCGGGATCATTCC | | | |
| | CCCTCATGCTACACTGGTCTTCGA | | | |
| | TGTGGAGCTGCTGAAGCTGGAA | | | |
| Linker | GGATCTGGACCGCGG | 1519 | GSGPR | 1520 |
| T2A | GAAGGCCGAGGGAGCCTGCTGA | 1521 | EGRGSLLTCGDVEENPGP | 1522 |
| | CATGTGGCGATGTGGAGGAAAAC | | | |
| | CCAGGACCA | | | |
| Linker | CCACGG | 1523 | PR | 1524 |
| Signal | ATGGAGTTTGGACTTTCTTGGTTG | 1525 | MEFGLSVVLFLVAILKGVQCSR | 1526 |
| Peptide | TTTTTGGTGGCAATTCTGAAGGGT | | | |
| | GTCCAGTGTAGCAGG | | | |
| FMC63 VL | GACATCCAGATGACACAGACTAC | 1527 | DIQMTQTTSSLSASLGDRVTISCR | 1528 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1529 | GGGSGGGG | 1530 |
| | GC | | | |
| FMC63 VH | GAGGTGAAACTGCAGGAGTCAGG | 1531 | EVKLQESGPGLVAPSQSLSVTCT | 1532 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1533 | GS | 1534 |
| CD34 | GAACTTCCTACTCAGGGGACTTTC | 1535 | ELPTQGTFSNVSTNVS | 1536 |
| epitope | TCAAACGTTAGCACAAACGTAAGT | | | |
| CD8 | CCCGCCCCAAGACCCCCCACACC | 1537 | PAPRPPTPAPTIASQPLSLRPEAC | 1538 |
| stalk | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1539 | IYIWAPLAGTCGVLLLSLVITLYCN | 1540 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1541 | VD | 1542 |
| CD3ζ | AGAGTGAAGTTCAGCAGGAGCGC | 1543 | RVKFSRSADAPAYQQGQNQLYN | 1544 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |

APPENDIX 16
pBP1440—pSFG-FKBPv.ΔC9.T2A-αCD19.Q.CD8stm.ζ.T2A.P2A-MC.FKBP_{wt}.FRB_{L}

| | | SEQ | | SEQ |
|---|---|---|---|---|
| | | ID | | ID |
| Fragment | Nucleotide | NO: | Peptide | NO: |
| MyD88 | ATGGCTGCAGGAGGTCCCGGCG | 1545 | MAAGGPGAGSAAPVSSTSSLPLA | 1546 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1547 | VE | 1548 |
| CD40 | AAAAAGGTGGCCAAGAAGCCAAC | 1549 | KKVAKKPTNKAPHPKQEPQEINF | 1550 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 1551 | VE | 1552 |
| FKBP_{WT}_{^{′}} | GGCGTCCAAGTCGAAACCATTAG | 1553 | GVQVETISPGDGRTFPKRGQTCV | 1554 |
| | TCCCGGCGATGGCAGAACATTTC | | VHYTGMLEDGKKFDSSRDRNKP | |
| | CTACAAGGGGACAAACATGTGTC | | FKFMLGKQEVIRGWEEGVAQMS | |
| | GTCCATTATACAGGCATGTTGGA | | VGQRAKLTISPDYAYGATGHPGII | |
| | GGACGGCAAAAAGTTCGACAGTA | | PPHATLVFDVELLKLE | |
| | GTAGAGATCGCAATAAACCTTTCA | | | |
| | AATTCATGTTGGGAAAACAAGAAG | | | |
| | TCATTAGGGGATGGGAGGAGGGC | | | |
| | GTGGCTCAAATGTCCGTCGGCCA | | | |
| | ACGCGCTAAGCTCACCATCAGCC | | | |
| | CCGACTACGCATACGGCGCTACC | | | |
| | GGACATCCCGGAATTATTCCCCC | | | |
| | TCACGCTACCTTGGTGTTTGACGT | | | |
| | CGAACTGTTGAAGCTCGAA | | | |
| Linker | GTCGAG | 1555 | VE | 1556 |
| FRB_{L} | CAATTGGAAATGTGGCATGAAGG | 1557 | QLEMWHEGLEEASRLYFGERNV | 1558 |
| | GTTGGAAGAAGCTTCAAGGCTGT | | KGMFEVLEPLHAMMERGPQTLK | |
| | ACTTCGGAGAGAGGAACGTGAAG | | ETSFNQAYGRDLMEAQEWCRKY | |
| | GGCATGTTTGAGGTTCTTGAACCT | | MKSGNVKDLLQAVVDLYYHVFRRI | |
| | CTGCACGCCATGATGGAACGGGG | | SK | |
| | ACCGCAGACACTGAAAGAAACCT | | | |
| | CTTTTAATCAGGCCTACGGCAGA | | | |
| | GACCTGATGGAGGCCCAAGAATG | | | |
| | GTGTAGAAAGTATATGAAATCCGG | | | |
| | TAACGTGAAAGACCTGCTCCAGG | | | |
| | CCTGGGACCTTTATTACCATGTGT | | | |
| | TCAGGCGGATCAGTAAG | | | |
| Linker | GGCTCAGGT | 1559 | GSG | 1560 |
| T2A | GAAGGCCGAGGGAGCCTGCTGA | 1561 | EGRGSLLTCGDVEENPGP | 1562 |
| | CATGTGGCGATGTGGAGGAAAAC | | | |
| | CCAGGACCA | | | |
| Linker | CCACGG | 1563 | PR | 1564 |
| Signal | ATGGAGTTTGGACTTTCTTGGTTG | 1565 | MEFGLSVVLFLVAILKGVQCSR | 1566 |
| Peptide | TTTTTGGTGGCAATTCTGAAGGGT | | | |
| | GTCCAGTGTAGCAGG | | | |
| FMC63 VL | GACATCCAGATGACACAGACTAC | 1567 | DIQMTQTTSSLSASLGDRVTISCR | 1568 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1569 | GGGSGGGG | 1570 |
| | GC | | | |
| FMC63 VH | GAGGTGAAACTGCAGGAGTCAGG | 1571 | EVKLQESGPGLVAPSQSLSVTCT | 1572 |
| | ACCTGGCCTGGTGGCGCCCTCAC | | VSGVSLPDYGVSWIRQPPRKGLE | |
| | AGAGCCTGTCCGTCACATGCACT | | WLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1573 | GS | 1574 |
| CD34 | GAACTTCCTACTCAGGGGACTTTC | 1575 | ELPTQGTFSNVSTNVS | 1576 |
| epitope | TCAAACGTTAGCACAAACGTAAGT | | | |
| CD8 stalk | CCCGCCCCAAGACCCCCCACACC | 1577 | PAPRPPTPAPTIASQPLSLRPEAC | 1578 |
| | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1579 | IYIWAPLAGTCGVLLLSLVITLYCN | 1580 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1581 | VD | 1582 |
| CD3ζ | AGAGTGAAGTTCAGCAGGAGCGC | 1583 | RVKFSRSADAPAYQQGQNQLYN | 1584 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | ggttccgga | 1585 | GSG | 1586 |
| T2A | GAAGGCCGAGGGAGCCTGCTGA | 1587 | EGRGSLLTCGDVEENPGP | 1588 |
| | CATGTGGCGATGTGGAGGAAAAC | | | |
| | CCAGGACCA | | | |
| Linker | ggatctgga | 1589 | GSG | 1590 |
| P2A | GCAACGAATTTTTCCCTGCTGAAA | 1591 | ATNFSLLKQAGDVEENPGP | 1592 |
| | CAGGCAGGGGACGTAGAGGAAAA | | | |
| | TCCTGGTCCT | | | |
| MyD88 | ATGGCTGCAGGAGGTCCCGGCG | 1593 | MAAGGPGAGSAAPVSSTSSLPLA | 1594 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1595 | VE | 1596 |
| CD40 | AAAAAGGTGGCCAAGAAGCCAAC | 1597 | KKVAKKPTNKAPHPKQEPQEINF | 1598 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 1599 | VE | 1600 |
| Linker | GTCGAG | 1601 | VE | 1602 |
| STOPtail | TCAGGCGGTGGCTCAGGTCCGC | 1603 | SGGGSGPR-STOP | 1604 |
| | GGTGA | | | |

APPENDIX 17
pBP1460—pSFG-FKBPv.ΔC9.T2A-αCD19.Q.CD8stm.ζ.T2A.P2A-MC.FKBP_{wt}.FRB_{L}

| | | SEQ | | SEQ |
|---|---|---|---|---|
| | | ID | | ID |
| Fragment | Nucleotide | NO: | Peptide | NO: |
| Leader | ATGCTCGAGCAATTGGAG | 1605 | MLEQLE | 1606 |
| peptide | | | | |
| FKBPv | GGAGTGCAGGTGGAGACTATTAG | 1607 | GVQVETISPGDGRTFPKRGQTCV | 1608 |
| | CCCCGGAGATGGCAGAACATTCC | | VHYTGMLEDGKKVDSSRDRNKP | |
| | CCAAAAGAGGACAGACTTGCGTC | | FKFMLGKQEVIRGVVEEGVAQMS | |
| | GTGCATTATACTGGAATGCTGGAA | | VGQRAKLTISPDYAYGATGHPGII | |
| | GACGGCAAGAAGGTGGACAGCA | | PPHATLVFDVELLKLE | |
| | GCCGGGACCGAAACAAGCCCTTC | | | |
| | AAGTTCATGCTGGGGAAGCAGGA | | | |
| | AGTGATCCGGGGCTGGGAGGAA | | | |
| | GGAGTCGCACAGATGTCAGTGGG | | | |
| | ACAGAGGGCCAAACTGACTATTA | | | |
| | GCCCAGACTACGCTTATGGAGCA | | | |
| | ACCGGCCACCCCGGGATCATTCC | | | |
| | CCCTCATGCTACACTGGTCTTCGA | | | |
| | TGTGGAGCTGCTGAAGCTGGAA | | | |
| Linker | TCAGGCGGTGGCTCAGGTGTGGA | 1609 | SGGGSGVD | 1610 |
| | C | | | |
| Δcaspase9 | GGATTTGGTGATGTCGGTGCTCT | 1611 | GFGDVGALESLRGNADLAYILSM | 1612 |
| | TGAGAGTTTGAGGGGAAATGCAG | | EPCGHCLIINNVNFCRESGLRTRT | |
| | ATTTGGCTTACATCCTGAGCATGG | | GSNIDCEKLRRRFSSLHFMVEVK | |
| | AGCCCTGTGGCCACTGCCTCATT | | GDLTAKKMVLALLELARQDHGAL | |
| | ATCAACAATGTGAACTTCTGCCGT | | DCCVVVILSHGCQASHLQFPGAV | |
| | GAGTCCGGGCTCCGCACCCGCA | | YGTDGCPVSVEKIVNIFNGTSCPS | |
| | CTGGCTCCAACATCGACTGTGAG | | LGGKPKLFFIQACGGEQKDHGFE | |
| | AAGTTGCGGCGTCGCTTCTCCTC | | VASTSPEDESPGSNPEPDATPFQ | |
| | GCTGCATTTCATGGTGGAGGTGA | | EGLRTFDQLDAISSLPTPSDIFVS | |
| | AGGGCGACCTGACTGCCAAGAAA | | YSTFPGFVSVVRDPKSGSVVYVET | |
| | ATGGTGCTGGCTTTGCTGGAGCT | | LDDIFEQWAHSEDLQSLLLRVAN | |
| | GGCGCGGCAGGACCACGGTGCT | | AVSVKGIYKQMPGCFNFLRKKLF | |
| | CTGGACTGCTGCGTGGTGGTCAT | | FKTSASRA | |
| | TCTCTCTCACGGCTGTCAGGCCA | | | |
| | GCCACCTGCAGTTCCCAGGGGCT | | | |
| | GTCTACGGCACAGATGGATGCCC | | | |
| | TGTGTCGGTCGAGAAGATTGTGA | | | |
| | ACATCTTCAATGGGACCAGCTGC | | | |
| | CCCAGCCTGGGAGGGAAGCCCA | | | |
| | AGCTCTTTTTCATCCAGGCCTGTG | | | |
| | GTGGGGAGCAGAAAGATCATGGG | | | |
| | TTTGAGGTGGCCTCCACTTCCCC | | | |
| | TGAAGACGAGTCCCCTGGCAGTA | | | |
| | ACCCCGAGCCAGATGCCACCCCG | | | |
| | TTCCAGGAAGGTTTGAGGACCTT | | | |
| | CGACCAGCTGGACGCCATATCTA | | | |
| | GTTTGCCCACACCCAGTGACATC | | | |
| | TTTGTGTCCTACTCTACTTTCCCA | | | |
| | GGTTTTGTTTCCTGGAGGGACCC | | | |
| | CAAGAGTGGCTCCTGGTACGTTG | | | |
| | AGACCCTGGACGACATCTTTGAG | | | |
| | CAGTGGGCTCACTCTGAAGACCT | | | |
| | GCAGTCCCTCCTGCTTAGGGTCG | | | |
| | CTAATGCTGTTTCGGTGAAAGGG | | | |
| | ATTTATAAACAGATGCCTGGTTGC | | | |
| | TTTAATTTCCTCCGGAAAAAACTT | | | |
| | TTCTTTAAAACATCAGCTAGCAGA | | | |
| | GCC | | | |
| Linker | GGATCTGGACCGCGG | 1613 | GSGPR | 1614 |
| T2A | GAAGGCCGAGGGAGCCTGCTGA | 1615 | EGRGSLLTCGDVEENPGP | 1616 |
| | CATGTGGCGATGTGGAGGAAAAC | | | |
| | CCAGGACCA | | | |
| Linker | CCACGG | 1617 | PR | 1618 |
| Signal | ATGGAGTTTGGACTTTCTTGGTTG | 1619 | MEFGLSWLFLVAILKGVQCSR | 1620 |
| Peptide | TTTTTGGTGGCAATTCTGAAGGGT | | | |
| | GTCCAGTGTAGCAGG | | | |
| FMC63 VL | GACATCCAGATGACACAGACTAC | 1621 | DIQMTQTTSSLSASLGDRVTISCR | 1622 |
| | ATCCTCCCTGTCTGCCTCTCTGG | | ASQDISKYLNVVYQQKPDGTVKLL | |
| | GAGACAGAGTCACCATCAGTTGC | | IYHTSRLHSGVPSRFSGSGSGTD | |
| | AGGGCAAGTCAGGACATTAGTAA | | YSLTISNLEQEDIATYFCQQGNTL | |
| | ATATTTAAATTGGTATCAGCAGAA | | PYTFGGGTKLEIT | |
| | ACCAGATGGAACTGTTAAACTCCT | | | |
| | GATCTACCATACATCAAGATTACA | | | |
| | CTCAGGAGTCCCATCAAGGTTCA | | | |
| | GTGGCAGTGGGTCTGGAACAGAT | | | |
| | TATTCTCTCACCATTAGCAACCTG | | | |
| | GAGCAAGAAGATATTGCCACTTAC | | | |
| | TTTTGCCAACAGGGTAATACGCTT | | | |
| | CCGTACACGTTCGGAGGGGGGA | | | |
| | CTAAGTTGGAAATAACA | | | |
| Flex | GGCGGAGGAAGCGGAGGTGGGG | 1623 | GGGSGGGG | 1624 |
| | GC | | | |
| FMC63 VH | GAGGTGAAACTGCAGGAGTCAGG | 1625 | EVKLQESGPGLVAPSQSLSVTCT | 1626 |
| | ACCTGGCCTGGTGGCGCCCTCAC | | VSGVSLPDYGVSWIRQPPRKGLE | |
| | AGAGCCTGTCCGTCACATGCACT | | VVLGVIWGSETTYYNSALKSRLTII | |
| | GTCTCAGGGGTCTCATTACCCGA | | KDNSKSQVFLKMNSLQTDDTAIY | |
| | CTATGGTGTAAGCTGGATTCGCC | | YCAKHYYYGGSYAMDYWGQGT | |
| | AGCCTCCACGAAAGGGTCTGGAG | | SVTVSS | |
| | TGGCTGGGAGTAATATGGGGTAG | | | |
| | TGAAACCACATACTATAATTCAGC | | | |
| | TCTCAAATCCAGACTGACCATCAT | | | |
| | CAAGGACAACTCCAAGAGCCAAG | | | |
| | TTTTCTTAAAAATGAACAGTCTGC | | | |
| | AAACTGATGACACAGCCATTTACT | | | |
| | ACTGTGCCAAACATTATTACTACG | | | |
| | GTGGTAGCTATGCTATGGACTACT | | | |
| | GGGGTCAAGGAACCTCAGTCACC | | | |
| | GTCTCCTCA | | | |
| Linker | GGATCC | 1627 | GS | 1628 |
| CD34 | GAACTTCCTACTCAGGGGACTTTC | 1629 | ELPTQGTFSNVSTNVS | 1630 |
| epitope | TCAAACGTTAGCACAAACGTAAGT | | | |
| CD8 | CCCGCCCCAAGACCCCCCACACC | 1631 | PAPRPPTPAPTIASQPLSLRPEAC | 1632 |
| stalk | TGCGCCGACCATTGCTTCTCAAC | | RPAAGGAVHTRGLDFACD | |
| | CCCTGAGTTTGAGACCCGAGGCC | | | |
| | TGCCGGCCAGCTGCCGGCGGGG | | | |
| | CCGTGCATACAAGAGGACTCGAT | | | |
| | TTCGCTTGCGAC | | | |
| CD8 | ATCTATATCTGGGCACCTCTCGCT | 1633 | IYIWAPLAGTCGVLLLSLVITLYCN | 1634 |
| trans- | GGCACCTGTGGAGTCCTTCTGCT | | HRNRRRVCKCPR | |
| membrane | CAGCCTGGTTATTACTCTGTACTG | | | |
| | TAATCACCGGAATCGCCGCCGCG | | | |
| | TTTGTAAGTGTCCCAGG | | | |
| Linker | GTCGAC | 1635 | VD | 1636 |
| CD3ζ | AGAGTGAAGTTCAGCAGGAGCGC | 1637 | RVKFSRSADAPAYQQGQNQLYN | 1638 |
| | AGACGCCCCCGCGTACCAGCAG | | ELNLGRREEYDVLDKRRGRDPE | |
| | GGCCAGAACCAGCTCTATAACGA | | MGGKPRRKNPQEGLYNELQKDK | |
| | GCTCAATCTAGGACGAAGAGAGG | | MAEAYSEIGMKGERRRGKGHDG | |
| | AGTACGATGTTTTGGACAAGAGA | | LYQGLSTATKDTYDALHMQALPP | |
| | CGTGGCCGGGACCCTGAGATGG | | R | |
| | GGGGAAAGCCGAGAAGGAAGAA | | | |
| | CCCTCAGGAAGGCCTGTACAATG | | | |
| | AACTGCAGAAAGATAAGATGGCG | | | |
| | GAGGCCTACAGTGAGATTGGGAT | | | |
| | GAAAGGCGAGCGCCGGAGGGGC | | | |
| | AAGGGGCACGATGGCCTTTACCA | | | |
| | GGGTCTCAGTACAGCCACCAAGG | | | |
| | ACACCTACGACGCCCTTCACATG | | | |
| | CAAGCTCTTCCACCTCGT | | | |
| Linker | ggttccgga | 1639 | GSG | 1640 |
| T2A | GAAGGCCGAGGGAGCCTGCTGA | 1641 | EGRGSLLTCGDVEENPGP | 1642 |
| | CATGTGGCGATGTGGAGGAAAAC | | | |
| | CCAGGACCA | | | |
| Linker | ggatctgga | 1643 | GSG | 1644 |
| P2A | GCAACGAATTTTTCCCTGCTGAAA | 1645 | ATNFSLLKQAGDVEENPGP | 1646 |
| | CAGGCAGGGGACGTAGAGGAAAA | | | |
| | TCCTGGTCCT | | | |
| MyD88 | ATGGCTGCAGGAGGTCCCGGCG | 1647 | MAAGGPGAGSAAPVSSTSSLPLA | 1648 |
| | CGGGGTCTGCGGCCCCGGTCTC | | ALNMRVRRRLSLFLNVRTQVAAD | |
| | CTCCACATCCTCCCTTCCCCTGG | | WTALAEEMDFEYLEIRQLETQAD | |
| | CTGCTCTCAACATGCGAGTGCGG | | PTGRLLDAWQGRPGASVGRLLD | |
| | CGCCGCCTGTCTCTGTTCTTGAA | | LLTKLGRDDVLLELGPSIEEDCQK | |
| | CGTGCGGACACAGGTGGCGGCC | | YILKQQQEEAEKPLQVAAVDSSV | |
| | GACTGGACCGCGCTGGCGGAGG | | PRTAELAGITTLDDPLGHMPERF | |
| | AGATGGACTTTGAGTACTTGGAG | | DAFICYCPSDI | |
| | ATCCGGCAACTGGAGACACAAGC | | | |
| | GGACCCCACTGGCAGGCTGCTG | | | |
| | GACGCCTGGCAGGGACGCCCTG | | | |
| | GCGCCTCTGTAGGCCGACTGCTC | | | |
| | GATCTGCTTACCAAGCTGGGCCG | | | |
| | CGACGACGTGCTGCTGGAGCTGG | | | |
| | GACCCAGCATTGAGGAGGATTGC | | | |
| | CAAAAGTATATCTTGAAGCAGCAG | | | |
| | CAGGAGGAGGCTGAGAAGCCTTT | | | |
| | ACAGGTGGCCGCTGTAGACAGCA | | | |
| | GTGTCCCACGGACAGCAGAGCTG | | | |
| | GCGGGCATCACCACACTTGATGA | | | |
| | CCCCCTGGGGCATATGCCTGAGC | | | |
| | GTTTCGATGCCTTCATCTGCTATT | | | |
| | GCCCCAGCGACATC | | | |
| Linker | GTCGAG | 1649 | VE | 1650 |
| CD40 | AAAAAGGTGGCCAAGAAGCCAAC | 1651 | KKVAKKPTNKAPHPKQEPQEINF | 1652 |
| | CAATAAGGCCCCCCACCCCAAGC | | PDDLPGSNTAAPVQETLHGCQP | |
| | AGGAGCCCCAGGAGATCAATTTT | | VTQEDGKESRISVQERQ | |
| | CCCGACGATCTTCCTGGCTCCAA | | | |
| | CACTGCTGCTCCAGTGCAGGAGA | | | |
| | CTTTACATGGATGCCAACCGGTC | | | |
| | ACCCAGGAGGATGGCAAAGAGAG | | | |
| | TCGCATCTCAGTGCAGGAGAGAC | | | |
| | AG | | | |
| Linker | GTCGAG | 1653 | VE | 1654 |
| FKBP_{WT}_{^{′}} | GGCGTCCAAGTCGAAACCATTAG | 1655 | GVQVETISPGDGRTFPKRGQTCV | 1656 |
| | TCCCGGCGATGGCAGAACATTTC | | VHYTGMLEDGKKFDSSRDRNKP | |
| | CTACAAGGGGACAAACATGTGTC | | FKFMLGKQEVIRGVVEEGVAQMS | |
| | GTCCATTATACAGGCATGTTGGA | | VGQRAKLTISPDYAYGATGHPGII | |
| | GGACGGCAAAAAGTTCGACAGTA | | PPHATLVFDVELLKLE | |
| | GTAGAGATCGCAATAAACCTTTCA | | | |
| | AATTCATGTTGGGAAAACAAGAAG | | | |
| | TCATTAGGGGATGGGAGGAGGGC | | | |
| | GTGGCTCAAATGTCCGTCGGCCA | | | |
| | ACGCGCTAAGCTCACCATCAGCC | | | |
| | CCGACTACGCATACGGCGCTACC | | | |
| | GGACATCCCGGAATTATTCCCCC | | | |
| | TCACGCTACCTTGGTGTTTGACGT | | | |
| | CGAACTGTTGAAGCTCGAA | | | |
| Linker | GTCGAG | 1657 | VE | 1658 |
| FRB_{L} | CAATTGGAAATGTGGCATGAAGG | 1659 | QLEMWHEGLEEASRLYFGERNV | 1660 |
| | GTTGGAAGAAGCTTCAAGGCTGT | | KGMFEVLEPLHAMMERGPQTLK | |
| | ACTTCGGAGAGAGGAACGTGAAG | | ETSFNQAYGRDLMEAQEWCRKY | |
| | GGCATGTTTGAGGTTCTTGAACCT | | MKSGNVKDLLQAWDLYYHVFRRI | |
| | CTGCACGCCATGATGGAACGGGG | | SK | |
| | ACCGCAGACACTGAAAGAAACCT | | | |
| | CTTTTAATCAGGCCTACGGCAGA | | | |
| | GACCTGATGGAGGCCCAAGAATG | | | |
| | GTGTAGAAAGTATATGAAATCCGG | | | |
| | TAACGTGAAAGACCTGCTCCAGG | | | |
| | CCTGGGACCTTTATTACCATGTGT | | | |
| | TCAGGCGGATCAGTAAG | | | |
| STOPtail | TCAGGCGGTGGCTCAGGTCCGC | 1661 | SGGGSGPR-STOP | 166 |
| | GGTGA | | | |

### Example 26: Dual-Switches to Control Activation and Elimination of Targeted Therapeutic Cells

The present Example provides methods related to controlling the activation and elimination of targeted therapeutic cells. The immune or therapeutic cells may be used for immunotherapy, where the therapeutic cells are targeted to solid tumor or leukemic cells, for example. Where certain methods provide data related to the use of T cells that express chimeric antigen receptors, it is understood that these methods may be modified for the use of other therapeutic cells, and heterologous polypeptides such as, for example, recombinant T cell receptors. Thus, for example, where the vectors and cells provided in this example may include the use of a CAR with an antigen recognition moiety directed against a particular antigen, or cell, the vectors and cells may be modified to include a use of a recombinant TCR directed against a particular antigen, or cell, by, for example, substituting the polynucleotide coding for the CAR with a polynucleotide coding for the recombinant TCR.

FIG. 68 provides results of assays comparing the costimulatory ability of T cells that co-express a first generation CAR and either a rapamycin/rapalog, or a rimiducid-inducible chimeric truncated MyD88/CD40 polypeptide (MC) in T cells. For these assays, the rapalog-inducible MC (MC-Rap or iRMC) comprised a wild-type FKBP12 polypeptide (F_{wt}) and a FRB_{L} polypeptide (F_{L}); the rimiducid-inducible MC (iMC+CARζ, or iMC) comprised two FKBP12ᵥ36 polypeptides (Fᵥ) (FIG. 68B). The assay compared MCRap and iMC directed costimulation on CAR-T cell killing of tumor cells. Human PBMCs containing mostly T cells were activated and transduced with retrovirus vectors pBP1455 encoding a PSCA directed first generation CAR downstream of a rapalog responsive costimulatory domain (MyD88-CD40-FKBP-FRB_{L}, termed MC-Rap), retrovirus pBP0189 in which costimulation is imparted by iMC (MyD88-CD40-FKBP_{V36}-FKBP_{V36}) or with a control retrovirus construct encoding the CAR, but no costimulatory molecules. After seven days of rest with IL-2, CAR-T cells were cocultured with PSCA expressing HPAC tumor cells labeled with Red Fluorescent Protein (RFP) at an effector to target ratio of 1:30. Growth of the labeled cells over one week was measured microscopically in an Incucyte chamber. In the presence of 2 nM C7-isobutyloxyrapamycin (IbuRap), MC-rap containing cells were able to control tumor cells as effectively as rimiducid stimulated iMC containing iMC+CARζ-T cells.

FIG. 69 provides results of assays comparing the costimulatory ability of T cells that co-express a first generation CAR, an MCRap polypeptide, and a rimiducid-inducible chimeric Caspase-9 polypeptide (iC9) from the same vector, where the placement of the polynucleotide that expresses the MCRap polypeptide is varied. The results provided in this assay demonstrate that the placement of MCRap within the three gene unified vector affects the degree of costimulatory activity. FIG. 69 provides a schematic representation of the various retrovirus vectors. pBP1466 places MC-Rap (MC-FKBP-FRB_{L}) 3′ to the CAR and iC9 safety switch. pBP1491 places MC-Rap between iC9 and the CAR. pBP1494 places MC-Rap 5′ to iC9 and the CAR. The CAR in each case contained an ScFV targeting the PSCA antigen. 2A cotranslational cleavage sequences separate MC-Rap from the CAR and from the iC9 apoptotic switch. FIG. 69B: provides a reporter assay of costimulatory signaling. 293 cells were transfected with 1 μg NF-κB-SeAP reporter and 3 μg of the indicated DNA constructs. After 24 hours, cultures were split to 12 wells of a 96 well plate and mock stimulated or treated with 2 nM rimiducid or 2 nM C7-isobutyloxyrapamycin in quadruplicate. Each transfection displayed minimal basal activity without stimulation while construct 1494 with MC-Rap positioned at the 5′ end of the retroviral construct displayed enhanced activity when stimulated with IbuRap. FIG. 69C provides results of CAR-T cytokine secretion assays. Human PBMCs containing mostly T cells were activated and transduced with retrovirus vectors indicated in (A). After seven days of rest with IL-2, CAR-T cells were cocultured with PSCA expressing HPAC tumor cells labeled with Red Fluorescent Protein (RFP) at an effector to target ratio of 1:5. 24 hours after the co-culture was established media was removed and interferon-Y⁻ levels determined by ELISA. Secretion of this cytokine is influenced both by signal 1 from the TCRζ component of the CAR and from costimulation through induced MC activity. This costimulation is most robust with IbuRap in construct 1494 with MC-Rap positioned at the 5′ end of the retroviral construct. FIG. 69D provides the results of CAR-T killing assays. Modified transduced or transfected T cells comprising polypeptides with the indicated topological orientations were cultured with HPAC-RFP tumor targets at an E:T ratio of 1:20 and growth of the labeled cells over one week was measured microscopically in an Incucyte chamber. In the presence of 2 nM C7-isobutyloxyrapamycin (IbuRap), construct 1494 with MC-Rap positioned at the 5′end was most effective in drug dependent tumor control. (Not shown) In each case, activation of the safety switch iC9 with rimiducid incubation caused CAR-T apoptosis and a loss of tumor control.

FIG. 70 provides results of assays comparing the costimulatory ability of T cells that co-express a first generation CAR, an MCRap polypeptide, and a rimiducid-inducible chimeric Caspase-9 polypeptide (iC9) from the same vector, where the orientation and positioning of the polynucleotide that expresses the MCRap polypeptide is varied. The orientation and positioning of FRB and FKBP was modified to compare MC costimulatory activity in the T cell that expressed the vector. FIG. 70A provides a Schematic representation of retroviral vectors. BP1493 and BP1494 places FKBP and FRB_{L} 3′ to MC and in that orientation. pBP1796 maintains the same orientation of FKBP relative to FRB but places these drug binding components at the 5′ end of the construct thus making an amino terminal fusion. Constructs BP1757 and BP1759 reverse the orientation of FRB and FKBP placing FRB_{L} at the amino terminus. The antigens targeted by the ScFV units of the CARs are indicated. FIG. 70B provides results of reporter assays assay of costimulatory signaling. 293 cells were transfected with 1 μg NF-κB-SeAP reporter and 3 μg of the indicated DNA constructs. After 24 hours, cultures were split 96 well plates and a dilution series of C7-isobutyloxyrapamycin added in quadruplicate. Each transfection displayed minimal basal activity without stimulation while construct 1757 displayed enhanced stimulation with the rapalog. FIGS. 70C and 70D provide results of PSCA-CAR-T killing assays. T cells with the indicated topological orientations of FRB_{L}, FKBP and MC were cultured with HPAC-RFP tumor targets at an E:T ratio of 1:20 (C) or 1:30 (D) and growth of the labeled cells over one week was measured microscopically in an Incucyte chamber. In the presence of 2 nM C7-isobutyloxyrapamycin (IbuRap), construct 1757 with MC-Rap positioned at the 5′end was most effective in tumor control without the addition of drug. Increased potency with drug was indicated at high E:T of 1:30 where only 1757 was able to proliferate sufficiently to maintain tumor control. FIGS. 70E, 70F, and 70G provide results of HER2-CAR-T killing assays. T cells with the indicated topological orientations of FRB_{L}, FKBP and MC were cultured with HPAC-RFP tumor targets at an E:T ratio of 1:15 (FIG. 70E), SKOV3 ovarian cancer cells (E:T=1:10) (FIG. 70F) or SKBR3-GFP breast cancer cells (E:T=1:1) (FIG. 70G) and growth of the labeled cells over one week was measured microscopically in an Incucyte chamber. In the presence of 2 nM C7-isobutyloxyrapamycin (IbuRap), construct 1759 with MC-Rap positioned at the 5′end was most effective in tumor control without the addition of drug. Increased potency with drug was indicated at high E:T of 1:30 where only 1757 was able to proliferate sufficiently to maintain tumor control. From these data it is concluded that maximal drug dependent MC-Rap potency is effected by positioning FRB then FKBP amino terminal to MC.

FIG. 71 provides results of assays that assay the apoptotic activity of T cells that co-express a first generation CAR, an MCRap polypeptide, and an iC9 polypeptide. The assays provide results showing that in these cells, the inducible apoptosis is only directed by dimerization of iC9 with rimiducid. PBMCs containing mostly T cells were activated and transduced with the indicated retroviral constructs and a control construct BP1488 that carries only MC-Rap with the CAR and no iC9. Cells were incubated with caspase 3/7 activity indicator reagent (Essen Biosciences) in an Incucyte incubator/microscope with increasing quantities of rimiducid (FIG. 71A) or C7-isobutyloxyrapamycin (FIG. 71B). At very low concentrations of rimiducid (<100 μM), the FKBPᵥ₃₆-caspase9 (iC9) component was observed to be activated from each construct but not from the MC-Rap CAR-T cells (1488) not containing iC9. Even high concentrations of IbuRap over 100 fold above the level used to activate MC-rap (normally 1 nM is used) are insufficient to activate apoptosis indicating that complex rapamycin directed heterodimerization events between coexpressed MC-FKBP-FRB_{L} and FKBP-Caspase that are theoretically possible, are not evident in this assay.

FIG. 72 provides schematic diagrams of a dual-switch iMC plus iRC9, in the form of single retroviral vector, or in two retroviral vectors. FIG. 72A provides a schematic of a unified vector design that amalgamates both the iMC activation switch (FᵥFᵥ) (present at the 3′ end of the vector) and the iRC9 (FRB and FKBP_{wt}) which is present in the vector at the 5′ end. Transduced T cells are marked with the Q-bend 10 (Q) epitope derived from CD34. The CombiCAR platform (FIG. 72B) includes the same protein components, but expressed from two retroviruses to increase the expression level of iMC and thereby the potency of the construct. iRC9 is marked by the expression of a truncated form of CD19 that contains only the extracellular domain and no intracellular signaling domain. The iMC+CARζ component incorporates iMC for costimulation and the CAR cistron which contains the Q epitope marker immediately following the ScFV.

FIG. 73A provides the results of assays of apoptosis activity in cells that express the iRC9 polypeptide, where the orientation and positioning of FRB and FKBPwt are varied. FIG. 73A provides schematic representations of iRC9 retroviral constructs BP1501 is a negative control containing only the caspase9 component without a drug-binding moiety. BP0220 is a iC9 construct in which FKBPᵥ is attached to caspase 9 producing iC9. This construct is responsive to rimiducid and not rapamycin. Constructs BP1310 and BP1311 have wild-type FKBP (to which rimiducid has poor affinity) and FRB in the indicated orientations. FIG. 73B provides results of assays of T cells transduced with various retroviral constructs of FIG. 73A. PBMCs containing mostly T cells were activated and transduced with the indicated retroviral constructs and cells were incubated with caspase 3/7 activity indicator reagent (Essen Biosciences) in an Incucyte incubator/microscope for 24 h with increasing quantities of rapamycin. Fluorescent conversion of the cells indicates cleavage of the caspase 3/7 reagent to mark apoptosis over time. FIG. 73C is a graphical representation of the maximal apoptotic activity relative to the commencement of drug treatment from the assays of FIG. 73B, as a function of rapamycin concentration. iRC9 is most effective when FRB is positioned amino-terminal to FKBP12 and caspase-9. FIG. 73D provides a Western blot of Caspase-9 transgene expression in T cells. Cells from two donors transduced with the indicated retroviral vectors were lysed and protein extracted, resolved on an SDS polyacrylamide gel, transferred to a PVDF filter and caspase-9 expression visualized by western blot. Consistent with the higher rapamycin-induced apoptotic activity of BP1310, expression was slightly higher than that of BP1311.

FIG. 74 provides results of assays comparing the activation profile of iMC+CARζ-T cells (cells express iMC and CAR) with CombiCAR-T cells (cells express iMC, CAR, and iRC9). To determine if inclusion of the chimeric caspase polypeptide from BP1311 impairs iMC+CARζ-T cell efficacy, human PBMCs were activated and transduced with the indicated retrovirus vectors. After seven days of rest with IL-2, CAR-T cells were cocultured with PSCA expressing HPAC tumor cells labeled with Red Fluorescent Protein (RFP) at an effector to target ratio of 1:10. 48 hours after the co-culture was established media was removed and interleukin-6 (IL-6, FIG. 74A), IL-2 (FIG. 74B), and interferon-Y (IFN-Y, FIG. 74C) levels determined by ELISA. Cytokine secretion was augmented by rimiducid treatment in a dose-dependent fashion and was closely similar between iMC+CARζ and CombiCAR formats. Interestingly CombiCAR was somewhat less effective to stimulate IFN secretion. FIG. 74D provides the results of a CAR-T killing assay. CAR-T cells in the indicated formats with the indicated topological orientations were cultured with HPAC-RFP tumor targets at an E:T ratio of 1:10. Growth of the labeled cells over one week was measured microscopically in an Incucyte chamber. At this level of CAR-T inclusion killing was not dependent on drug but was enhanced by basal activity of iMC (compare each CAR format with BP1373 which lacks iMC). FIG. 74E provides a Western blot of expression of iMC and chimeric caspase polypeptide in each CAR format. T Cells transduced with the indicated retroviral vectors were lysed and protein extracted, resolved on an SDS polyacrylamide gel, transferred to a PVDF filter and expression of the indicated proteins probed by western blot. Vinculin expression represents the equality of loading of each lane in the gel. Expression of iMC was similar between iMC+CARζ and CombiCAR formats.

FIG. 75 provides the results of assays of rapamycin-inducible Caspase-9 (iRC9) within unified-single- and dual-vector formats. T cells from two separate donors (877 and 904) were (anti-CD3/CD28) activated and non-transduced (NT) or transduced with retroviruses encoding CD34 epitope-marked iMC+CARζ-T (iMC-2A-CAR-zeta), (iMC-2A-iRC9-2A-CAR-zeta), or CombiCAR (co-transduction with viruses encoding iMC+CARζ-T and iRC9). A population of 5×10⁷ iMC+CARζ-T cells (1463) and T cells (1358) were enriched for transduced cells by purification with a CD34 microbead kit (Miltenyi) while CombiCAR cells were selected with CD19 microbeads that identified the marker from the chimeric caspase construct. This enrichment procedure, or ‘sorting’ of highly transduced cells yielded greater than 95% marker positivity. In FIG. 75A, cells were incubated with a Caspase 3/7 activity indicator (Essen Biosciences) in an IncuCyte plate incubator/microscope with 0, 1, or 10 nM rapamycin. Readings of apoptosis (via Caspase-3/7 activation) were automatically conducted every 4 hours and shown for unsorted (top panel) and sorted (bottom panel) cells. FIG. 75B provides graphical representations of data for both donors (and average values) at the 12-hour timepoint for unsorted (left panel) and sorted (right panel) cells. For FIG. 75C, similarly transduced T cells were incubated for 24 hours in the presence of 0, 1, or 10 nM rapamycin and stained with Annexin V and propidium iodide (PI) for cell death. Representative graphs of unsorted cells from 1 donor are shown. FIG. 75D provides graphical representations of the results of both donors from unsorted (left panel) and sorted (right panel) cells treated for 24 hours as in FIG. 75C.

FIG. 76 provides the results of in vivo experiments assessing the efficacy of different forms of iMC co-expressed in T cells with an anti-CD123 CAR directed against acute myelogenous leukemia tumors. The iMC was assessed in the form of a iMC+CARζ-T cell that does not express the iRC9 safety switch, and in the form of the dual-switch CombiCAR platform, where the cells also express iRC9. FIG. 76A provides micrographs of tumor-bearing animals determined by bioluminescence (BLI) imaging. 1.0×10⁶ GFP-Luciferase-expressing THP-1 tumor cells were injected i.v. into age-matched NSG mice. Seven days later (day 0), 2.5×10⁶ non-transduced (NT), iMC+CARζ-transduced, or CombiCAR-transduced (i.e., dual-transduced cells with iMC+CARζ-T and iRC9 vectors., marked by CD34 or CD19-derived epitopes, respectively) T cells were injected into tumor-bearing animals. Groups (n=5) were injected with rimiducid (1 mg/kg) at day 1 and day 15. Animals were imaged weekly starting on the day of T cell injection (day 0). Transduced CombiCAR cells were CD19-selected and normalized for CAR expression via CD34. FIG. 76B provides data showing the average tumor growth per group (left panel), reflected via BLI (Radiance) or % weight change post-T cell injection (right panel) is shown. FIG. 76C provides data showing the number of human T cells in spleens at termination (day 28). Left panel shows total number of human (murine(m)CD45⁻CD3⁺) T cells before or after rimiducid (AP) injection. Middle panel shows the % of human T cells with detectable CAR expression (via CD34 epitope). Right panel shows the % of human T cells with detectable iRC9 (via CD19 epitope). *=p<0.05 by Student's T test. FIG. 76D provides data showing the vector copy number (VCN) determined by qPCR from DNA derived from spleen (top) or bone marrow (bottom). Primers were chosen specific for iMC (left panels) or iCaspase-9 (right panels). *=p<0.05 by Student's T test.

FIG. 77 provides the results of in vivo experiments assessing the efficacy of different forms of iMC co-expressed in T cells with an anti-CD33 CAR directed against MOLM13 tumors. The iMC was assessed in the form of a iMC+CARζ-T cell that does not express the iRC9 safety switch, and in the form of the dual-switch CombiCAR platform, where the cells also express iRC9. FIG. 77A provides micrographs of tumor-bearing animals determined by BLI imaging. PBMCs were activated and co-transduced with retroviruses derived from the anti-CD33 iMC+CARζ-T vector (pBP1293) and the iRC9 vector (pB1385). NSG mice were engrafted with 1×10⁶ MOLM13-GFP.Fluc cells i.v. for 6 days followed by i.v. infusion of 5×10⁶ iRC9 or CD33-CombiCAR-expressing T cells. Rimiducid or placebo were given i.p. weekly after T cell infusion at 1 mg/kg. In FIG. 77A, GFP.Fluc growth was measured using IVIS bioluminescence (BLI) and average radiance was calculated (FIG. 77B). FIG. 77C provides the results of Kaplan-Meier analysis from the in vivo assay of FIG. 77A. FIG. 77D provides the results of representative FACS analysis of the rimiducid-treated CD33 CombiCAR group at termination on day 32 after T cell injection.

FIG. 78 provides the results of assays comparing the specificity and efficacy of the rimiducid inducible iC9 and rapamycin-inducible (iRC9) apoptotic switches in a whole animal model. 1.0×10⁷ T cells transduced with BP220 (containing iC9) or BP1310 (containing iRC9) and with a GFP-luciferase vector were implanted intravenously into 8-week-old female, immune-deficient mice (NOD.CgPrkdc^{scid}Il2^{tm1wjl}/SzJ; NSG). Mice were subjected to IVIS imaging ˜4 hrs after T cell injection (˜14 hrs post-drug administration). The following day, mice were imaged just before drug injection (0 hrs), then injected IP with vehicle, rimiducid diluted in solutol and PBS, or rapamycin diluted in 10% PEG, 17% Tween-80. Mice were imaged again at 5-6 hrs, and 24 hrs after drug injection. Mice were sacrificed and spleens were removed for FACS analysis. FIG. 78A provides the results of BLI assays. Mice were imaged for firefly luciferase-derived bioluminescence by IVIS. Mice were imaged at the indicated time points relative to administration of drug or vehicle. Because rimiducid is specific for the F36V mutant of FKBP12 and the iC9 utilizes wild-type FKBP12, loss of radiance by T cell apoptosis is only observed with rimiducid treatment of the iC9 and not iC9 bearing animals. FIG. 78B provides a graphical representation of the average calculated radiance from FIG. 78A. FIG. 78C provides data showing the results of independent quantitative analyses of the in vivo assays of FIG. 78A. Human T cells in mice spleens were isolated and single-cell suspensions were made by lysing red cells with ammonia chloride/potassium (ACK)-based lysis buffer followed by mechanical dissociation through a 70-μm nylon filter. Cells were subsequently stained with the following antibodies: anti-hCD3-PerCP.Cy5.5, anti-hCD19-APC, and anti-mCD45RA-BV510. Human T cell counts were normalized to the number of mouse CD45 expressing cells present in the spleen preparations.

FIG. 79 provides the results of dose responsiveness assays of the rapamycin induced iC9 apoptotic switch in a whole animal model. 1.0×10⁷ T cells transduced with BP1385 (containing iRC9) and with a GFP-luciferase vector were implanted intravenously into 8-week-old female, immune-deficient mice (NOD.CgPrkdc^{scid}Il2^{tm1Wjl}/SzJ; NSG). Mice were subjected to IVIS imaging ˜4 hrs after T cell injection (˜24 hrs post-drug administration). The following day, mice were imaged just before drug injection (0 hrs), then injected IP with vehicle, rimiducid diluted in solutol and PBS or rapamycin diluted in 5% PEG, 2.5% Tween-80 at the step-log dilutions from 10 mg/kg body weight. Mice were imaged again at 5-6 hrs, and 24 hrs after drug injection. Mice were sacrificed and spleens were removed for FACS analysis. FIG. 79A provides a pictorial representation of BL1 imaging. FIG. 79B provides a graphical representation of the average calculated radiance from FIG. 79A. FIG. 79C provides graphs of the number of human T cells in spleens at termination (24 hours). Left panel shows total number of human (murine(m)CD45⁻CD3⁺) that are marked with CD19 indicating presence of the apoptotic switch. Middle panel shows the mean fluorescence intensity for the CD19 marker in the human T cells remaining in the spleen. Right panel shows the total number of human T cells with detectable iC9 (via CD19 epitope). *=p<0.05 by Student's T test. FIG. 79D provides graphs of vector copy number (VCN) determined by qPCR from DNA derived from spleen. Primers were chosen specific for iMC (left panel, a negative control in this experiment) or iCaspase-9 and GFP-luc (middle and right panels).

### Example 27: A Dual-Switch Platform to Control CAR-T Cell Efficacy and Safety with Two Independent, Non-Toxic Chemical Inducers of Protein Dimerization

The present example discusses the use of a single retroviral vector to express an iRMC polypeptide, a first generation CAR, and an iC9 safety switch. For this example, a rapalog, C7-isobutyloxyrapamycin (Ibu-Rap) was used to induce MC activity. It is understood that wild type FRB and rapamycin may also be used in the present example. Also, for this example, the iRMC comprised a modified FRB polypeptide, called FRB_{KLW} or “KLW”. In other examples of the present technology, the iRC9 and iRMC polypeptides may comprise modified FRB polypeptides rather than the wild type FRBs provided herein. Also, various rapalogs that bind to the wild type or modified FRB polypeptides may be used to activate iRC9 or iRMC.

Chimeric Antigen Receptor (CAR) T cell strategies have demonstrated effectiveness against multiple disseminated cancers, but solid tumors remain a challenge. To improve efficacy a platform was developed to separate tumor antigen-specific first generation CARs from a cytosolic costimulatory component, iRMC, regulated by a non-immunosuppressive analog of rapamycin, C7-isobutyloxyrapamycin (IBuRap). To mitigate the risk of off-tumor cytotoxicity or excessive cytokine release, iRMC was combined with the Caspase-9-based switch, iC9, directing rapid T cell apoptosis by rimiducid-regulated homodimerization and activation.

To produce a non-immunosuppressive rapamycin analog (rapalog), the acid-sensitive C7-methoxy group was replaced with an isobutyloxy moiety. The added bulk of this ‘bump’ reduced affinity and inhibition for mTOR/TORC1 but retained subnanomolar affinity for a mutant FKBP-Rapamycin Binding (FRB) domain, termed KLW, derived from mTOR. KLW was fused in-tandem with wild-type FKBP12 and the costimulatory signaling domains MyD88 and CD40 to create iRMC. NF-κB activity was stimulated in a robust and dose-dependent fashion (EC₅₀<1 nM) with iBuRap. When incorporated into a retroviral (iRMC-2A-iC9-2A-CAR) format and incubated with CAR-specific tumor cells, IBuRAP addition stimulated T cell proliferation, cytokine production and dose-dependent tumor cell killing. In 7-day cocultures, rapalog/iRMC-stimulated HER2-specific iRMC-2A-iC9-2A-CAR T cells preferentially proliferated, leading to elimination of >90% of SKBR3 breast carcinoma cells (E:T, 1:1), SKOV3 ovarian carcinoma (E:T, 1:5), or HPAC (E:T, 1:15) pancreatic carcinoma cells. If rimiducid was included in iRMC-2A-iC9-2A-CAR-T cultures, T cell apoptosis was rapidly induced (T_{1/2}=6 hours for microscopic observation of fluorescent caspase-3 substrate). Despite the fact that both iRMC and iC9 incorporated FKBP12 domains, because rimiducid is highly specific for the F36V variant of FKBP12, the costimulatory and safety switches are orthogonally regulated.

### Example 28: Dual-Switches to Target Solid Tumors

The present example discusses the use of a two retroviral vectors, where the first vector expresses an iMC and a first generation CAR, and the second vector expresses a iRC9 safety switch.

While chimeric antigen receptor (CAR) T immunotherapies have shown remarkable efficacy against leukemias and lymphomas, improved CAR-T efficacy and persistence without compromising safety are needed to overcome solid tumors. Two independently regulated molecular switches were developed that can elicit specific and rapid induction of cellular responses upon exposure to their cognate ligands. Cell activation is controlled by the homodimerizer rimiducid that triggers signaling cascades downstream of MyD88 and CD40 (iMC). A rapamycin-controlled pro-apoptotic switch is co-expressed, which induces dimerization of caspase-9 to mitigate possible toxicity from excessive CAR-T function (iRC9). When combined with a first generation CAR, these molecular switches allow for specific and efficient regulation of engineered T cells.

T cells were activated and co-transduced with the “iMC+CARζ”, SFG-iMC-2A-CAR.ζ vector, and a iC9-X vector, SFG-FRB.FKBP12.C9-2A-ΔCD19 to create a CombiCAR. The observed rapid kinetics and -95% efficiency of rapamycin-dependent cell death was determined by caspase-3 activation and annexin V conversion. In vivo assessment of iC9-X functionality was performed with EGFPluciferase (EGFPluc)-labeled T cells in NSG mice, showing that rapamycin treatment caused cell death in 90% of iRMC-containing T cells within 24 hours, similar to clinically validated rimiducid-regulated iC9.

iMC costimulation was further evaluated in a 7-day tumor cell coculture by cytokine production, T cell growth and tumor cell killing. Addition of iC9-X did not deleteriously affect antitumor efficacy of rimiducid-treated iMC-containing CAR-T cells, which eliminated 0E-19 esophageal tumor cells in a coculture assay at a 1:20 effector to target ratio (3.9±4.3% 0E19-GFP.Ffluc cells remained in iMC+CARζ-modified cultures 1.1±0.1% for CombiCAR), or T cell expansion (53.4±9.4% CAR⁺ for iMC+CARζ vs 44.6±13.2% for CombiCAR). In vivo efficacy of the CombiCAR-T cells was evaluated weekly in NSG mice implanted with EGFPluc-marked tumor burden and for T cell persistence via a Renilla luciferase marker. When challenged in a OE9 tumor-bearing mouse model, anti-HER2 dual-switch T cells controlled tumor growth in a rimiducid-dependent manner, which was representative of multiple tumor models.

The dual-switch platform comprising separate ligand dependent activation and apoptosis and a first generation CAR, efficiently controlled T cell growth and tumor elimination when costimulation was provided via systemic administration of rimiducid. Deployment of iC9-X results in rapid and efficient elimination of CombiCAR-T cells, providing a user-controlled system for managing persistence and safety of tumor antigen-specific CAR-T cells.

### Example 29: Dual-Switches to Activate Recombinant TCR-Expressing Cells

The present example discusses the use of a two retroviral vectors, where the first vector expresses an iMC and a recombinant TCR directed against PRAME, and the second vector expresses a iRC9 safety switch.

T cells engineered to express the α and β chains of antigen-specific T cell receptors (TCRs) have shown promise as a cancer immunotherapy treatment; however, durable responses have been limited by poor persistence of gene-modified T cells. Additionally, severe toxicities, including patient deaths, have occurred upon infusion of large numbers of TCR-modified T cells. To enhance T cell persistence while providing a safeguard against life-threatening toxicity, a dual-switch αβ TCR platform was developed that uses a rapamycin (Rap)-induced caspase-9 (iRC9) together with a rimiducid (Rim)-controlled activation switch, inducible MyD88/CD40 (iMC).

The αβ TCR sequence derived from an HLA-A2-restricted, PRAME-specific T cell clone was synthesized and placed in-frame with iMC, comprising signaling domains from MyD88 and CD40 fused to tandem Rim-binding mutant FKBP12v36 domains to generate the iMC-PRAME TCR. Caspase-9 was fused to FRB and wild-type FKBP domains and cloned in-frame with a selectable marker, truncated CD19 (ΔCD19) to generate iRC9-ΔCD19 retrovirus. All modules were separated by 2A polypeptide sequences. Activated human T cells were dual-transduced with iMC-PRAME TCR and iRC9-ΔCD19 viruses and subsequently enriched for CD19 expression using magnetic columns. iMC and iRC9 were activated by exposing transduced T cells to 10 nM Rim or Rap, respectively. Proliferation, cytokine production and cytotoxicity of TCR-modified T cells were assessed in co-culture assays with U266 (myeloma) and THP-1 (AML) cells in presence or absence of inducible ligands.

T cells transduced with iMC-PRAME TCR and iRC9-ΔCD19 showed efficient and stable expression for TCR and ΔCD19 post-CD19 selection (82±9% CD3⁺Vβ1⁺, 96±2% CD3⁺CD19⁺). In coculture assays, dual-switch PRAME TCR demonstrated specific lysis of HLA-A2⁺PRAME⁺ THP-1 and U266 tumor cells compared to an irrelevant TCR (CMVpp65) with or without iMC activation. However, Rim exposure induced a 42-fold induction of IL-2 (9±0.3 versus 385±180 μg/ml IL-2) and resulted in 13-fold expansion of TCR-modified T cells. The expression of iRC9 did not interfere with TCR function, nor with the synergy between TCR and iMC activation. Further, exposure to Rap triggered rapid apoptosis of dual-switch TCR-modified T cells (72±5% Annexin-V⁺ with Rap versus 14±4% without drug) indicating that the suicide switch is also functional.

iMC utilizes rimiducid to provide costimulation to TCR-engineered T cells. In addition, iRC9 is provides a rapamycin-inducible suicide switch that can eliminate T cells in case of severe toxicity. This iMC-enhanced iRC9-incorporating TCR is a prototype of novel dual-switch TCR-engineered T cell therapies that may increase efficacy, durability and safety of adoptive T cell therapies.

The following Appendices provide sequences and plasmids referred to in Examples provided herein:

APPENDIX 18
pBP1293—pSFG-iMC.T2A-αhCD33(My9.6).ζ

| | | SEQ | | SEQ |
|---|---|---|---|---|
| | | ID | | ID |
| Fragment | Nucleotide | NO: | Peptide | NO: |
| MyD88 | atggctgcaggaggtcccggcgcggggtctgcgg | 1663 | MAAGGPGAGSAAPVSSTSSLPLA | 1664 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 1665 | VE | 1666 |
| CD40 | aaaaaggtggccaagaagccaaccaataaggc | 1667 | KKVAKKPTNKAPHPKQEPQEINF | 1668 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 1669 | VE | 1670 |
| FKBP_{V}_{^{′}} | ggcgtccaagtcgaaaccattagtcccggcgatg | 1671 | GVQVETISPGDGRTFPKRGQTCV | 1672 |
| | gcagaacatttcctaaaaggggacaaacatgtgt | | VHYTGMLEDGKKVDSSRDRNKP | |
| | cgtccattatacaggcatgttggaggacggcaaa | | FKFMLGKQEVIRGWEEGVAQMS | |
| | aaggtggacagtagtaGaGAtcGcAAtAAa | | VGQRAKLTISPDYAYGATGHPGII | |
| | CCtTTcAAaTTcATGtTgGGaAAaCAa | | PPHATLVFDVELLKLE | |
| | GAaGTcATtaGgGGaTGGGAgGAgG | | | |
| | GcGTgGCtCAaATGtccGTcGGcCAac | | | |
| | GcGCtAAgCTcACcATcagcCCcGAcT | | | |
| | AcGCaTAcGGcGCtACcGGaCAtCCc | | | |
| | GGaATtATtCCcCCtCAcGCtACctTgG | | | |
| | TgTTtGAcGTcGAaCTgtTgAAgCTcGA | | | |
| | a | | | |
| Linker | gtcgag | 1673 | VE | 1674 |
| FKBP_{V} | ggagtgcaggtggagactatctccccaggagac | 1675 | GVQVETISPGDGRTFPKRGQTCV | 1676 |
| | gggcgcaccttccccaagcgcggccagacctgc | | VHYTGMLEDGKKVDSSRDRNKP | |
| | gtggtgcactacaccgggatgcttgaagatggaa | | FKFMLGKQEVIRGWEEGVAQMS | |
| | agaaagttgattcctcccgggacagaaacaagcc | | VGQRAKLTISPDYAYGATGHPGII | |
| | ctttaagtttatgctaggcaagcaggaggtgatccg | | PPHATLVFDVELLKLE | |
| | aggctgggaagaaggggttgcccagatgagtgtg | | | |
| | ggtcagagagccaaactgactatatctccagatta | | | |
| | tgcctatggtgccactgggcacccaggcatcatcc | | | |
| | caccacatgccactctcgtcttcgatgtggagcttct | | | |
| | aaaactggaa | | | |
| Linker | ccgcGG | 1677 | PR | 1678 |
| T2A | GAGGGCAGAGGCAGCCTCCTGAC | 1679 | EGRGSLLTCGDVEENPGP | 1680 |
| | ATGTGGGGACGTCGAGGAGAACC | | | |
| | CTGGCCCA | | | |
| Linker | CCTTGG | 1681 | PW | 1682 |
| Signal | ATGGAGTTCGGATTGAGCTGGCTG | 1683 | MEFGLSVVLFLVAILKGVQCSR | 1684 |
| Peptide | TTCCTGGTGGCAATACTCAAGGGC | | | |
| | GTTCAATGTTCACGG | | | |
| My9-6 VL | GAAATTGTGCTGACTCAGAGCCCG | 1685 | EIVLTQSPGSLAVSPGERVTMSC | 1686 |
| | GGTAGCCTGGCCGTGTCCCCCGG | | KSSQSVFFSSSQKNYLAVVYQQIP | |
| | AGAGCGAGTGACCATGAGCTGTAA | | GQSPRLLIYWASTRESGVPDRFT | |
| | ATCCAGCCAATCAGTTTTTTTTTCA | | GSGSGTDFTLTISSVQPEDLAIYY | |
| | TCATCTCAAAAAAACTATCTGGCAT | | CHQYLSSRTFGQGTKLEIKR | |
| | GGTACCAACAGATACCCGGGCAGT | | | |
| | CCCCACGGCTGCTGATTTACTGGG | | | |
| | CATCAACACGCGAGAGCGGTGTG | | | |
| | CCCGACAGATTCACCGGAAGCGG | | | |
| | GAGCGGCACGGACTTCACACTTAC | | | |
| | CATCTCAAGCGTACAACCGGAGGA | | | |
| | CTTGGCTATCTATTACTGCCACCAA | | | |
| | TATCTTTCCTCCAGAACATTCGGAC | | | |
| | AGGGAACGAAACTGGAGATCAAAA | | | |
| | GA | | | |
| Flex | GGCGGCGGGAGTGGGGGAGGAG | 1687 | gggsgggg | 1688 |
| | GT | | | |
| Linker | CAGGTG | 1689 | qv | 1690 |
| My9-6 VH | CAGGTGCAGCTGCAGCAGCCTGG | 1691 | QVQLQQPGAEVVKPGASVKMSC | 1692 |
| | AGCCGAGGTGGTGAAGCCCGGCG | | KASGYTFTSYYIHWIKQTPGQGL | |
| | CATCTGTGAAAATGTCTTGCAAGG | | EVVVGVIYPGNDDISYNQKFQGKA | |
| | CAAGCGGATATACATTTACTAGCTA | | TLTADKSSTTAYMQLSSLTSEDS | |
| | CTACATCCATTGGATCAAGCAAAC | | AVYYCAREVRLRYFDVWGQGTT | |
| | CCCCGGACAGGGCCTCGAATGGG | | VTVSS | |
| | TGGGAGTTATTTACCCGGGGAACG | | | |
| | ATGATATCTCTTATAATCAGAAATT | | | |
| | CCAAGGGAAAGCCACCCTGACTGC | | | |
| | AGACAAATCAAGTACCACAGCCTA | | | |
| | TATGCAGCTCAGCTCCCTGACAAG | | | |
| | CGAGGATTCCGCTGTGTACTACTG | | | |
| | TGCCAGGGAGGTTAGACTTCGATA | | | |
| | TTTTGATGTTTGGGGGCAGGGAAC | | | |
| | TACCGTGACCGTGAGCAGC | | | |
| Linker | GGCTCC | 1693 | gs | 1694 |
| CD34 | GAGCTGCCAACCCAGGGAACTTTT | 1695 | ELPTQGTFSNVSTNVS | 1696 |
| epitope | TCAAATGTATCAACTAACGTCTCA | | | |
| CD8 | CCCGCGCCACGACCACCAACACC | 1697 | PAPRPPTPAPTIASQPLSLRPEAC | 1698 |
| stalk | AGCCCCAACCATTGCATCCCAGCC | | RPAAGGAVHTRGLDFACD | |
| | TTTGTCTCTCCGGCCCGAGGCTTG | | | |
| | TCGCCCCGCCGCCGGGGGTGCCG | | | |
| | TCCATACCCGAGGCCTGGACTTCG | | | |
| | CCTGCGAT | | | |
| CD8 | ATATATATTTGGGCTCCTCTGGCC | 1699 | IYIWAPLAGTCGVLLLSLVITLYCN | 1700 |
| trans- | GGTACCTGCGGCGTACTGCTCCTG | | HRNRRRVCKCPR | |
| membrane | TCACTGGTAATAACCCTGTATTGCA | | | |
| | ATCACAGGAACAGAAGGAGAGTCT | | | |
| | GTAAGTGCCCCCGC | | | |
| Linker | GTCGAC | 1701 | VD | 1702 |
| CD3ζ | AGAGTGAAGTTCAGCAGGAGCGCA | 1703 | RVKFSRSADAPAYQQGQNQLYN | 1704 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |
| STOP | TGA | 1705 | stop | |

APPENDIX 19
pBP1296—pSFG-iMC.T2A-αhCD123(32716).ζ

| | | SEQ | | SEQ |
|---|---|---|---|---|
| Fragment | Nucleotide | ID NO: | Peptide | ID NO: |
| MyD88 | atggctgcaggaggtcccggcgcggggtctgcgg | 1706 | MAAGGPGAGSAAPVSSTSSLPLA | 1707 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 1708 | VE | 1709 |
| CD40 | aaaaaggtggccaagaagccaaccaataaggc | 1710 | KKVAKKPTNKAPHPKQEPQEINF | 1711 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 1712 | VE | 1713 |
| FKBP_{V}_{^{′}} | ggcgtccaagtcgaaaccattagtcccggcgatg | 1714 | GVQVETISPGDGRTFPKRGQTCV | 1715 |
| | gcagaacatttcctaaaaggggacaaacatgtgt | | VHYTGMLEDGKKVDSSRDRNKP | |
| | cgtccattatacaggcatgttggaggacggcaaa | | FKFMLGKQEVIRGWEEGVAQMS | |
| | aaggtggacagtagtaGaGAtcGcAAtAAa | | VGQRAKLTISPDYAYGATGHPGII | |
| | CCtTTcAAaTTcATGtTgGGaAAaCAa | | PPHATLVFDVELLKLE | |
| | GAaGTcATtaGgGGaTGGGAgGAgG | | | |
| | GcGTgGCtCAaATGtccGTcGGcCAac | | | |
| | GcGCtAAgCTcACcATcagcCCcGAcT | | | |
| | AcGCaTAcGGcGCtACcGGaCAtCCc | | | |
| | GGaATtATtCCcCCtCAcGCtACctTgG | | | |
| | TgTTtGAcGTcGAaCTgtTgAAgCTcGA | | | |
| | a | | | |
| Linker | gtcgag | 1716 | VE | 1717 |
| FKBP_{V} | ggagtgcaggtggagactatctccccaggagac | 1718 | GVQVETISPGDGRTFPKRGQTCV | 1719 |
| | gggcgcaccttccccaagcgcggccagacctgc | | VHYTGMLEDGKKVDSSRDRNKP | |
| | gtggtgcactacaccgggatgcttgaagatggaa | | FKFMLGKQEVIRGWEEGVAQMS | |
| | agaaagttgattcctcccgggacagaaacaagcc | | VGQRAKLTISPDYAYGATGHPGII | |
| | ctttaagtttatgctaggcaagcaggaggtgatccg | | PPHATLVFDVELLKLE | |
| | aggctgggaagaaggggttgcccagatgagtgtg | | | |
| | ggtcagagagccaaactgactatatctccagatta | | | |
| | tgcctatggtgccactgggcacccaggcatcatcc | | | |
| | caccacatgccactctcgtcttcgatgtggagcttct | | | |
| | aaaactggaa | | | |
| Linker | CCGCGG | 1720 | PR | 1721 |
| T2A | GAGGGCAGAGGCAGCCTCCTGAC | 1722 | EGRGSLLTCGDVEENPGP | 1723 |
| | ATGTGGGGACGTCGAGGAGAACC | | | |
| | CTGGCCCA | | | |
| Linker | CCTTGG | 1724 | PW | 1725 |
| Signal | ATGGAGTTCGGATTGAGCTGGCTG | 1726 | MEFGLSVVLFLVAILKGVQCSR | 1727 |
| Peptide | TTCCTGGTGGCAATACTCAAGGGC | | | |
| | GTTCAATGTTCACGG | | | |
| CD123 | CAGATCCAACTGGTGCAGTCAGGC | 1728 | QIQLVQSGPELKKPGETVKISCKA | 1729 |
| (32716) | CCGGAACTGAAGAAGCCAGGGGA | | SGYIFTNYGMNVVVKQAPGKSFK | |
| VH | GACAGTCAAAATAAGTTGTAAAGC | | WMGWINTYTGESTYSADFKGRF | |
| | CAGCGGCTACATATTTACTAATTAC | | AFSLETSASTAYLHINDLKNEDTA | |
| | GGGATGAATTGGGTGAAGCAAGC | | TYFCARSGGYDPMDYWGQGTS | |
| | GCCGGGCAAATCCTTTAAATGGAT | | VTV | |
| | GGGGTGGATAAACACATACACAGG | | | |
| | AGAGTCAACGTACAGCGCGGACTT | | | |
| | CAAAGGTCGATTCGCGTTCAGTCT | | | |
| | CGAGACCAGCGCGAGTACAGCTTA | | | |
| | CCTCCACATCAACGATCTTAAAAAC | | | |
| | GAAGACACGGCAACCTATTTTTGC | | | |
| | GCCCGGTCAGGCGGTTACGACCC | | | |
| | TATGGACTATTGGGGCCAAGGGAC | | | |
| | CTCCGTTACGGTA | | | |
| Flex | TCTTCAGGCGGTGGCGGGAGTGG | 1730 | SSGGGGSGGGGSGGGGS | 1731 |
| | TGGAGGAGGCTCAGGCGGCGGGG | | | |
| | GATCA | | | |
| CD123 | GACATCGTACTGACCCAATCTCCC | 1732 | DIVLTQSPASLAVSLGQRATISCR | 1733 |
| (32716) | GCTAGCCTTGCAGTATCCTTGGGT | | ASESVDNYGNTFMHVVYQQKPG | |
| VL | CAACGCGCTACAATAAGTTGCCGG | | QPPKLLIYRASNLESGIPARFSGS | |
| | GCTAGTGAGTCCGTAGACAACTAT | | GSRTDFTLTINPVEADDVATYYC | |
| | GGCAACACCTTCATGCATTGGTAC | | QQSNEDPPTFGAGTKLELKESKY | |
| | CAACAAAAACCAGGTCAGCCACCC | | GPPCP | |
| | AAACTTCTCATTTACAGAGCGTCTA | | | |
| | ATCTCGAAAGCGGCATCCCTGCTC | | | |
| | GATTCTCTGGAAGCGGAAGTAGAA | | | |
| | CCGACTTTACACTGACTATAAACCC | | | |
| | CGTCGAAGCCGATGATGTTGCCAC | | | |
| | TTATTACTGTCAACAGAGCAATGAG | | | |
| | GACCCACCGACATTCGGTGCTGGT | | | |
| | ACCAAGCTGGAGTTGAAGGAGTCA | | | |
| | AAATACGGGCCTCCCTGTCCC | | | |
| Linker | GGCTCC | 1734 | gs | 1735 |
| CD34 | GAGCTGCCAACCCAGGGAACTTTT | 1736 | ELPTQGTFSNVSTNVS | 1737 |
| epitope | TCAAATGTATCAACTAACGTCTCA | | | |
| CD8 stalk | CCCGCGCCACGACCACCAACACC | 1738 | PAPRPPTPAPTIASQPLSLRPEAC | 1739 |
| | AGCCCCAACCATTGCATCCCAGCC | | RPAAGGAVHTRGLDFACD | |
| | TTTGTCTCTCCGGCCCGAGGCTTG | | | |
| | TCGCCCCGCCGCCGGGGGTGCCG | | | |
| | TCCATACCCGAGGCCTGGACTTCG | | | |
| | CCTGCGAT | | | |
| CD8 | ATATATATTTGGGCTCCTCTGGCC | 1740 | IYIWAPLAGTCGVLLLSLVITLYCN | 1741 |
| trans- | GGTACCTGCGGCGTACTGCTCCTG | | HRNRRRVCKCPR | |
| membrane | TCACTGGTAATAACCCTGTATTGCA | | | |
| | ATCACAGGAACAGAAGGAGAGTCT | | | |
| | GTAAGTGCCCCCGC | | | |
| Linker | GTCGAC | 1742 | VD | 1743 |
| CD3ζ | AGAGTGAAGTTCAGCAGGAGCGCA | 1744 | RVKFSRSADAPAYQQGQNQLYN | 1745 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |
| STOP | TGA | 1746 | stop | |

APPENDIX 20
pBP1327—pSFG-FRB.FKBP_{V}.ΔC9.2A-ΔCD19

| | | SEQ | | SEQ |
|---|---|---|---|---|
| Fragment | Nucleotide | ID NO: | Peptide | ID NO: |
| FRB | gaaatgTGGCATGAAGGGTTGGAAG | 1747 | EMWHEGLEEASRLYFGERNVKG | 1748 |
| | AAGCTTCAAGGCTGTACTTCGGAG | | MFEVLEPLHAMMERGPQTLKETS | |
| | AGAGGAACGTGAAGGGCATGTTTG | | FNQAYGRDLMEAQEWCRKYMK | |
| | AGGTTCTTGAACCTCTGCACGCCA | | SGNVKDLTQAVVDLYYHVFRRISK | |
| | TGATGGAACGGGGACCGCAGACA | | | |
| | CTGAAAGAAACCTCTTTTAATCAGG | | | |
| | CCTACGGCAGAGACCTGATGGAG | | | |
| | GCCCAAGAATGGTGTAGAAAGTAT | | | |
| | ATGAAATCCGGTAACGTGAAAGAC | | | |
| | CTGactCAGGCCTGGGACCTTTATT | | | |
| | ACCATGTGTTCAGGCGGATCAGTA | | | |
| | AG | | | |
| Linker | TCAGGCGGTGGCTCAGGT | 1749 | SGGGSG | 1750 |
| FKBP_{V} | GGcGTcCAaGTcGAaACcATtagtCCc | 1751 | GVQVETISPGDGRTFPKRGQTCV | 1752 |
| | GGcGAtGGcaGaACaTTtCCtAAaaGg | | VHYTGMLEDGKKVDSSRDRNKP | |
| | GGaCAaACaTGtGTcGTcCAtTAtACa | | FKFMLGKQEVIRGWEEGVAQMS | |
| | GGcATGtTgGAgGAcGGcAAaAAggT | | VGQRAKLTISPDYAYGATGHPGII | |
| | CGAcagtagtaGaGAtcGcAAtAAaCCtT | | PPHATLVFDVELLKL | |
| | TcAAaTTcATGtTgGGaAAaCAaGAaG | | | |
| | TcATtaGgGGaTGGGAgGAgGGcGTg | | | |
| | GCtCAaATGtccGTcGGcCAacGcGCt | | | |
| | AAgCTcACcATcagcCCcGAcTAcGCa | | | |
| | TAcGGcGCtACcGGaCAtCCcggaattA | | | |
| | TtCCcCCtCAcGCtACctTgGTgTTtGAc | | | |
| | GTcGAaCTgtTgAAgCTc | | | |
| Linker | TCGGGGGGCGGATCAGG | 1753 | SGGGS | 1754 |
| Δcaspase9 | GTCGACGGATTTGGTGATGTCGGT | 1755 | VDGFGDVGALESLRGNADLAYIL | 1756 |
| | GCTCTTGAGAGTTTGAGGGGAAAT | | SMEPCGHCLIINNVNFCRESGLR | |
| | GCAGATTTGGCTTACATCCTGAGC | | TRTGSNIDCEKLRRRFSSLHFMV | |
| | ATGGAGCCCTGTGGCCACTGCCTC | | EVKGDLTAKKMVLALLELARQDH | |
| | ATTATCAACAATGTGAACTTCTGCC | | GALDCCVVVILSHGCQASHLQFP | |
| | GTGAGTCCGGGCTCCGCACCCGC | | GAVYGTDGCPVSVEKIVNIFNGT | |
| | ACTGGCTCCAACATCGACTGTGAG | | SCPSLGGKPKLFFIQACGGEQKD | |
| | AAGTTGCGGCGTCGCTTCTCCTCG | | HGFEVASTSPEDESPGSNPEPDA | |
| | CTGCATTTCATGGTGGAGGTGAAG | | TPFQEGLRTFDQLDAISSLPTPSD | |
| | GGCGACCTGACTGCCAAGAAAATG | | IFVSYSTFPGFVSWRDPKSGSVVY | |
| | GTGCTGGCTTTGCTGGAGCTGGC | | VETLDDIFEQWAHSEDLQSLLLR | |
| | GCgGCAGGACCACGGTGCTCTGG | | VANAVSVKGIYKQMPGCFNFLRK | |
| | ACTGCTGCGTGGTGGTCATTCTCT | | KLFFKTSASRA | |
| | CTCACGGCTGTCAGGCCAGCCAC | | | |
| | CTGCAGTTCCCAGGGGCTGTCTAC | | | |
| | GGCACAGATGGATGCCCTGTGTCG | | | |
| | GTCGAGAAGATTGTGAACATCTTC | | | |
| | AATGGGACCAGCTGCCCCAGCCT | | | |
| | GGGAGGGAAGCCCAAGCTCTTTTT | | | |
| | CATCCAGGCCTGTGGTGGGGAGC | | | |
| | AGAAAGACCATGGGTTTGAGGTGG | | | |
| | CCTCCACTTCCCCTGAAGACGAGT | | | |
| | CCCCTGGCAGTAACCCCGAGCCA | | | |
| | GATGCCACCCCGTTCCAGGAAGGT | | | |
| | TTGAGGACCTTCGACCAGCTGGAC | | | |
| | GCCATATCTAGTTTGCCCACACCC | | | |
| | AGTGACATCTTTGTGTCCTACTCTA | | | |
| | CTTTCCCAGGTTTTGTTTCCTGGAG | | | |
| | GGACCCCAAGAGTGGCTCCTGGTA | | | |
| | CGTTGAGACCCTGGACGACATCTT | | | |
| | TGAGCAGTGGGCTCACTCTGAAGA | | | |
| | CCTGCAGTCCCTCCTGCTTAGGGT | | | |
| | CGCTAATGCTGTTTCGGTGAAAGG | | | |
| | GATTTATAAACAGATGCCTGGTTG | | | |
| | CTTTAATTTCCTCCGGAAAAAACTT | | | |
| | TTCTTTAAAACATCAGCTAGCAGAG | | | |
| | CC | | | |
| Linker | ccgcGG | 1757 | PR | 1758 |
| T2A | GAAGGCCGAGGGAGCCTGCTGAC | 1759 | EGRGSLLTCGDVEENPGP | 1760 |
| | ATGTGGCGATGTGGAGGAAAACCC | | | |
| | AGGACCA | | | |
| ΔCD19 | ATGCCACCACCTCGCCTGCTGTTC | 1761 | MPPPRLLFFLLFLTPMEVRPEEPL1762 | |
| | TTTCTGCTGTTCCTGACACCTATGG | | VVKVEEGDNAVLQCLKGTSDGPT | |
| | AGGTGCGACCTGAGGAACCACTG | | QQLTWSRESPLKPFLKLSLGLPG | |
| | GTCGTGAAGGTCGAGGAAGGCGA | | LGIHMRPLAIVVLFIFNVSQQMGGF | |
| | CAATGCCGTGCTGCAGTGCCTGAA | | YLCQPGPPSEKAWQPGVVTVNVE | |
| | AGGCACTTCTGATGGGCCAACTCA | | GSGELFRWNVSDLGGLGCGLKN | |
| | GCAGCTGACCTGGTCCAGGGAGT | | RSSEGPSSPSGKLMSPKLYVWA | |
| | CTCCCCTGAAGCCTTTTCTGAAACT | | KDRPEIVVEGEPPCLPPRDSLNQS | |
| | GAGCCTGGGACTGCCAGGACTGG | | LSQDLTMAPGSTLVVLSCGVPPD | |
| | GAATCCACATGCGCCCTCTGGCTA | | SVSRGPLSVVTHVHPKGPKSLLSL | |
| | TCTGGCTGTTCATCTTCAACGTGA | | ELKDDRPARDMVVVMETGLLLPR | |
| | GCCAGCAGATGGGAGGATTCTACC | | ATAQDAGKYYCHRGNLTMSFHL | |
| | TGTGCCAGCCAGGACCACCATCCG | | EITARPVLVVHVVLLRTGGVVKVSA | |
| | AGAAGGCCTGGCAGCCTGGATGG | | VTLAYLIFCLCSLVGILHLQRALVL | |
| | ACCGTCAACGTGGAGGGGTCTGG | | RRKRKRMTDPTRRF | |
| | AGAACTGTTTAGGTGGAATGTGAG | | | |
| | TGACCTGGGAGGACTGGGATGTG | | | |
| | GGCTGAAGAACCGCTCCTCTGAAG | | | |
| | GCCCAAGTTCACCCTCAGGGAAGC | | | |
| | TGATGAGCCCAAAACTGTACGTGT | | | |
| | GGGCCAAAGATCGGCCCGAGATC | | | |
| | TGGGAGGGAGAACCTCCATGCCT | | | |
| | GCCACCTAGAGACAGCCTGAATCA | | | |
| | GAGTCTGTCACAGGATCTGACAAT | | | |
| | GGCCCCCGGGTCCACTCTGTGGC | | | |
| | TGTCTTGTGGAGTCCCACCCGACA | | | |
| | GCGTGTCCAGAGGCCCTCTGTCCT | | | |
| | GGACCCACGTGCATCCTAAGGGG | | | |
| | CCAAAAAGTCTGCTGTCACTGGAA | | | |
| | CTGAAGGACGATCGGCCTGCCAG | | | |
| | AGACATGTGGGTCATGGAGACTGG | | | |
| | ACTGCTGCTGCCACGAGCAACCGC | | | |
| | ACAGGATGCTGGAAAATACTATTG | | | |
| | CCACCGGGGCAATCTGACAATGTC | | | |
| | CTTCCATCTGGAGATCACTGCAAG | | | |
| | GCCCGTGCTGTGGCACTGGCTGC | | | |
| | TGCGAACCGGAGGATGGAAGGTC | | | |
| | AGTGCTGTGACACTGGCATATCTG | | | |
| | ATCTTTTGCCTGTGCTCCCTGGTG | | | |
| | GGCATTCTGCATCTGCAGAGAGCC | | | |
| | CTGGTGCTGCGGAGAAAGAGAAA | | | |
| | GAGAATGACTGACCCAACAAGAAG | | | |
| | GTTT | | | |
| STOP | TGA | 1763 | stop | |

APPENDIX 21
pBP1328—pSFG-FKBP_{V}.FRB.ΔC9.2A-ΔCD19

| | | SEQ | | SEQ |
|---|---|---|---|---|
| Fragment | Nucleotide | ID NO: | Peptide | ID NO: |
| FKBP_{V} | GGcGTcCAaGTcGAaACcATtagtCCc | 1764 | GVQVETISPGDGRTFPKRGQTCV | 1765 |
| | GGcGAtGGcaGaACaTTtCCtAAaaGg | | VHYTGMLEDGKKVDSSRDRNKP | |
| | GGaCAaACaTGtGTcGTcCAtTAtACa | | FKFMLGKQEVIRGWEEGVAQMS | |
| | GGcATGtTgGAgGAcGGcAAaAAggT | | VGQRAKLTISPDYAYGATGHPGII | |
| | CGAcagtagtaGaGAtcGcAAtAAaCCtT | | PPHATLVFDVELLKL | |
| | TcAAaTTcATGtTgGGaAAaCAaGAaG | | | |
| | TcATtaGgGGaTGGGAgGAgGGcGTg | | | |
| | GCtCAaATGtccGTcGGcCAacGcGCt | | | |
| | AAgCTcACcATcagcCCcGAcTAcGCa | | | |
| | TAcGGcGCtACcGGaCAtCCcggaattA | | | |
| | TtCCcCCtCAcGCtACctTgGTgTTtGAc | | | |
| | GTcGAaCTgtTgAAgCTc | | | |
| Linker | TCGGGGGGCGGATCAGG | 1766 | SGGGS | 1767 |
| FRB | gaaatgTGGCATGAAGGGTTGGAAG | 1768 | EMWHEGLEEASRLYFGERNVKG | 1769 |
| | AAGCTTCAAGGCTGTACTTCGGAG | | MFEVLEPLHAMMERGPQTLKETS | |
| | AGAGGAACGTGAAGGGCATGTTTG | | FNQAYGRDLMEAQEWCRKYMK | |
| | AGGTTCTTGAACCTCTGCACGCCA | | SGNVKDLTQAVVDLYYHVFRRISK | |
| | TGATGGAACGGGGACCGCAGACA | | | |
| | CTGAAAGAAACCTCTTTTAATCAGG | | | |
| | CCTACGGCAGAGACCTGATGGAG | | | |
| | GCCCAAGAATGGTGTAGAAAGTAT | | | |
| | ATGAAATCCGGTAACGTGAAAGAC | | | |
| | CTGactCAGGCCTGGGACCTTTATT | | | |
| | ACCATGTGTTCAGGCGGATCAGTA | | | |
| | AG | | | |
| Linker | TCAGGCGGTGGCTCAGGT | 1770 | SGGGSG | 1771 |
| Δcaspase9 | GTCGACGGATTTGGTGATGTCGGT | 1772 | VDGFGDVGALESLRGNADLAYIL | 1773 |
| | GCTCTTGAGAGTTTGAGGGGAAAT | | SMEPCGHCLIINNVNFCRESGLR | |
| | GCAGATTTGGCTTACATCCTGAGC | | TRTGSNIDCEKLRRRFSSLHFMV | |
| | ATGGAGCCCTGTGGCCACTGCCTC | | EVKGDLTAKKMVLALLELARQDH | |
| | ATTATCAACAATGTGAACTTCTGCC | | GALDCCVVVILSHGCQASHLQFP | |
| | GTGAGTCCGGGCTCCGCACCCGC | | GAVYGTDGCPVSVEKIVNIFNGT | |
| | ACTGGCTCCAACATCGACTGTGAG | | SCPSLGGKPKLFFIQACGGEQKD | |
| | AAGTTGCGGCGTCGCTTCTCCTCG | | HGFEVASTSPEDESPGSNPEPDA | |
| | CTGCATTTCATGGTGGAGGTGAAG | | TPFQEGLRTFDQLDAISSLPTPSD | |
| | GGCGACCTGACTGCCAAGAAAATG | | IFVSYSTFPGFVSWRDPKSGSVVY | |
| | GTGCTGGCTTTGCTGGAGCTGGC | | VETLDDIFEQWAHSEDLQSLLLR | |
| | GCgGCAGGACCACGGTGCTCTGG | | VANAVSVKGIYKQMPGCFNFLRK | |
| | ACTGCTGCGTGGTGGTCATTCTCT | | KLFFKTSASRA | |
| | CTCACGGCTGTCAGGCCAGCCAC | | | |
| | CTGCAGTTCCCAGGGGCTGTCTAC | | | |
| | GGCACAGATGGATGCCCTGTGTCG | | | |
| | GTCGAGAAGATTGTGAACATCTTC | | | |
| | AATGGGACCAGCTGCCCCAGCCT | | | |
| | GGGAGGGAAGCCCAAGCTCTTTTT | | | |
| | CATCCAGGCCTGTGGTGGGGAGC | | | |
| | AGAAAGACCATGGGTTTGAGGTGG | | | |
| | CCTCCACTTCCCCTGAAGACGAGT | | | |
| | CCCCTGGCAGTAACCCCGAGCCA | | | |
| | GATGCCACCCCGTTCCAGGAAGGT | | | |
| | TTGAGGACCTTCGACCAGCTGGAC | | | |
| | GCCATATCTAGTTTGCCCACACCC | | | |
| | AGTGACATCTTTGTGTCCTACTCTA | | | |
| | CTTTCCCAGGTTTTGTTTCCTGGAG | | | |
| | GGACCCCAAGAGTGGCTCCTGGTA | | | |
| | CGTTGAGACCCTGGACGACATCTT | | | |
| | TGAGCAGTGGGCTCACTCTGAAGA | | | |
| | CCTGCAGTCCCTCCTGCTTAGGGT | | | |
| | CGCTAATGCTGTTTCGGTGAAAGG | | | |
| | GATTTATAAACAGATGCCTGGTTG | | | |
| | CTTTAATTTCCTCCGGAAAAAACTT | | | |
| | TTCTTTAAAACATCAGCTAGCAGAG | | | |
| | CC | | | |
| Linker | ccgcGG | 1774 | PR | 1775 |
| T2A | GAAGGCCGAGGGAGCCTGCTGAC | 1776 | EGRGSLLTCGDVEENPGP | 1777 |
| | ATGTGGCGATGTGGAGGAAAACCC | | | |
| | AGGACCA | | | |
| ΔCD19 | ATGCCACCACCTCGCCTGCTGTTC | 1778 | MPPPRLLFFLLFLTPMEVRPEEPL | 1779 |
| | TTTCTGCTGTTCCTGACACCTATGG | | VVKVEEGDNAVLQCLKGTSDGPT | |
| | AGGTGCGACCTGAGGAACCACTG | | QQLTWSRESPLKPFLKLSLGLPG | |
| | GTCGTGAAGGTCGAGGAAGGCGA | | LGIHMRPLAIVVLFIFNVSQQMGGF | |
| | CAATGCCGTGCTGCAGTGCCTGAA | | YLCQPGPPSEKAWQPGVVTVNVE | |
| | AGGCACTTCTGATGGGCCAACTCA | | GSGELFRWNVSDLGGLGCGLKN | |
| | GCAGCTGACCTGGTCCAGGGAGT | | RSSEGPSSPSGKLMSPKLYVWA | |
| | CTCCCCTGAAGCCTTTTCTGAAACT | | KDRPEIVVEGEPPCLPPRDSLNQS | |
| | GAGCCTGGGACTGCCAGGACTGG | | LSQDLTMAPGSTLVVLSCGVPPD | |
| | GAATCCACATGCGCCCTCTGGCTA | | SVSRGPLSVVTHVHPKGPKSLLSL | |
| | TCTGGCTGTTCATCTTCAACGTGA | | ELKDDRPARDMVVVMETGLLLPR | |
| | GCCAGCAGATGGGAGGATTCTACC | | ATAQDAGKYYCHRGNLTMSFHL | |
| | TGTGCCAGCCAGGACCACCATCCG | | EITARPVLVVHVVLLRTGGVVKVSA | |
| | AGAAGGCCTGGCAGCCTGGATGG | | VTLAYLIFCLCSLVGILHLQRALVL | |
| | ACCGTCAACGTGGAGGGGTCTGG | | RRKRKRMTDPTRRF | |
| | AGAACTGTTTAGGTGGAATGTGAG | | | |
| | TGACCTGGGAGGACTGGGATGTG | | | |
| | GGCTGAAGAACCGCTCCTCTGAAG | | | |
| | GCCCAAGTTCACCCTCAGGGAAGC | | | |
| | TGATGAGCCCAAAACTGTACGTGT | | | |
| | GGGCCAAAGATCGGCCCGAGATC | | | |
| | TGGGAGGGAGAACCTCCATGCCT | | | |
| | GCCACCTAGAGACAGCCTGAATCA | | | |
| | GAGTCTGTCACAGGATCTGACAAT | | | |
| | GGCCCCCGGGTCCACTCTGTGGC | | | |
| | TGTCTTGTGGAGTCCCACCCGACA | | | |
| | GCGTGTCCAGAGGCCCTCTGTCCT | | | |
| | GGACCCACGTGCATCCTAAGGGG | | | |
| | CCAAAAAGTCTGCTGTCACTGGAA | | | |
| | CTGAAGGACGATCGGCCTGCCAG | | | |
| | AGACATGTGGGTCATGGAGACTGG | | | |
| | ACTGCTGCTGCCACGAGCAACCGC | | | |
| | ACAGGATGCTGGAAAATACTATTG | | | |
| | CCACCGGGGCAATCTGACAATGTC | | | |
| | CTTCCATCTGGAGATCACTGCAAG | | | |
| | GCCCGTGCTGTGGCACTGGCTGC | | | |
| | TGCGAACCGGAGGATGGAAGGTC | | | |
| | AGTGCTGTGACACTGGCATATCTG | | | |
| | ATCTTTTGCCTGTGCTCCCTGGTG | | | |
| | GGCATTCTGCATCTGCAGAGAGCC | | | |
| | CTGGTGCTGCGGAGAAAGAGAAA | | | |
| | GAGAATGACTGACCCAACAAGAAG | | | |
| | GTTT | | | |
| STOP | TGA | 1780 | stop | |

APPENDIX 22
pBP1351—pSFG-SP163.FKBP.FRB.ΔC9.T2A-αhPSCA.Q.CD8stm.ζ.2A-iMC

| | | SEQ | | SEQ |
|---|---|---|---|---|
| | | ID | | ID |
| Fragment | Nucleotide | NO: | Peptide | NO: |
| QBI SP163 | AGCGCAGAGGCTTGGGGCAGCCG | 1781 | AQRLGAAERQPGPGPGLGSRRE | 1782 |
| | AGCGGCAGCCAGGCCCCGGCCCG | | RSPPRRARERAASQSEPERERE | |
| | GGCCTCGGTTCCAGAAGGGAGAG | | PRRPRTASET | |
| | GAGCCCGCCAAGGCGCGCAAGAG | | | |
| | AGCGGGCTGCCTCGCAGTCCGAG | | | |
| | CCGGAGAGGGAGCGCGAGCCGCG | | | |
| | CCGGCCCCGGACGGCCTCCGAAA | | | |
| | CC | | | |
| FKBP″ | GGcGTGCAaGTGGAaACTATaAGCC | 1783 | GVQVETISPGDGRTFPKRGQTCV | 1784 |
| | CgGGAGAcGGCcGcACATTtCCCAA | | VHYTGMLEDGKKFDSSRDRNKP | |
| | gAGAGGcCAGACcTGCGTgGTGCAc | | FKFMLGKQEVIRGWEEGVAQMS | |
| | TATACaGGAATGCTGGAgGACGGg | | VGQRAKLTISPDYAYGATGHPGII | |
| | AAGAAaTTCGAtAGCtcCCGGGAtCG | | PPHATLVFDVELLKLE | |
| | AAAtAAGCCtTTCAAaTTCATGCTGG | | | |
| | GcAAGCAaGAAGTcATCaGaGGCTG | | | |
| | GGAaGAAGGcGTCGCcCAGATGTC | | | |
| | cGTGGGtCAGcGcGCCAAgCTGACa | | | |
| | ATTAGtCCAGAtTACGCcTATGGcGC | | | |
| | AACaGGCCAtCCCGGcATCATcCCC | | | |
| | CCaCATGCcACACTcGTCTTtGATGT | | | |
| | cGAGCTcCTGAAaCTGGAg | | | |
| Linker | GGCGGGcaattg | 1785 | ggql | 1786 |
| FRB | gaaatgTGGCATGAAGGGTTGGAAG | 1787 | EMWHEGLEEASRLYFGERNVKG | 1788 |
| | AAGCTTCAAGGCTGTACTTCGGAG | | MFEVLEPLHAMMERGPQTLKETS | |
| | AGAGGAACGTGAAGGGCATGTTTG | | FNQAYGRDLMEAQEWCRKYMK | |
| | AGGTTCTTGAACCTCTGCACGCCA | | SGNVKDLTQAVVDLYYHVFRRISK | |
| | TGATGGAACGGGGACCGCAGACA | | | |
| | CTGAAAGAAACCTCTTTTAATCAGG | | | |
| | CCTACGGCAGAGACCTGATGGAG | | | |
| | GCCCAAGAATGGTGTAGAAAGTAT | | | |
| | ATGAAATCCGGTAACGTGAAAGAC | | | |
| | CTGactCAGGCCTGGGACCTTTATT | | | |
| | ACCATGTGTTCAGGCGGATCAGTA | | | |
| | AG | | | |
| Linker | TCAGGCGGTGGCTCAGGTccatgg | 1789 | SGGGSGPW | 1790 |
| Δcaspase9 | GGATTTGGTGATGTCGGTGCTCTT | 1791 | GFGDVGALESLRGNADLAYILSM | 1792 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 1793 | GSGPR | 1794 |
| T2A | GAAGGCCGAGGGAGCCTGCTGAC | 1795 | EGRGSLLTCGDVEENPGP | 1796 |
| | ATGTGGCGATGTGGAGGAAAACCC | | | |
| | AGGACCA | | | |
| Linker | CCATGG | 1797 | PW | 1798 |
| Signal | ATGGAGTTTGGACTTTCTTGGTTGT | 1799 | MEFGLSVVLFLVAILKGVQCSR | 1800 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 1801 | DIQLTQSPSTLSASMGDRVTITCS | 1802 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 1803 | gggsgggg | 1804 |
| | GC | | | |
| PSCA(A11) | GAGGTGCAGCTCGTGGAGTATGG | 1805 | EVQLVEYGGGLVQPGGSLRLSC | 1806 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 1807 | gs | 1808 |
| CD34 | GAACTTCCTACTCAGGGGACTTTC | 1809 | ELPTQGTFSNVSTNVS | 1810 |
| epitope | TCAAACGTTAGCACAAACGTAAGT | | | |
| CD8 stalk | CCCGCCCCAAGACCCCCCACACCT | 1811 | PAPRPPTPAPTIASQPLSLRPEAC | 1812 |
| | GCGCCGACCATTGCTTCTCAACCC | | RPAAGGAVHTRGLDFACD | |
| | CTGAGTTTGAGACCCGAGGCCTGC | | | |
| | CGGCCAGCTGCCGGCGGGGCCGT | | | |
| | GCATACAAGAGGACTCGATTTCGC | | | |
| | TTGCGAC | | | |
| CD8 | ATCTATATCTGGGCACCTCTCGCT | 1813 | IYIWAPLAGTCGVLLLSLVITLYCN | 1814 |
| trans- | GGCACCTGTGGAGTCCTTCTGCTC | | HRNRRRVCKCPR | |
| membrane | AGCCTGGTTATTACTCTGTACTGTA | | | |
| | ATCACCGGAATCGCCGCCGCGTTT | | | |
| | GTAAGTGTCCCAGG | | | |
| Linker | GTCGAC | 1815 | VD | 1816 |
| CD3ζ | AGAGTGAAGTTCAGCAGGAGCGCA | 1817 | RVKFSRSADAPAYQQGQNQLYN | 1818 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |
| Linker | gGAACGCGTGGATCGGGA | 1819 | gtrgsg | 1820 |
| P2A | GCTACTAACTTCAGCCTGCTGAAG | 1821 | ATNFSLLKQAGDVEENPGP | 1822 |
| | CAGGCTGGAGACGTGGAGGAGAA | | | |
| | Ccccgggcct | | | |
| MyD88 | atggctgcaggaggtcccggcgcggggtctgcgg | 1823 | MAAGGPGAGSAAPVSSTSSLPLA | 1824 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 1825 | VE | 1826 |
| CD40 | aaaaaggtggccaagaagccaaccaataaggc | 1827 | KKVAKKPTNKAPHPKQEPQEINF | 1828 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 1829 | VE | 1830 |
| FKBP_{V}_{^{′}} | GGcGTcCAaGTcGAaACcATtagtCCc | 1831 | GVQVETISPGDGRTFPKRGQTCV | 1832 |
| | GGcGAtGGcaGaACaTTtCCtAAaaGg | | VHYTGMLEDGKKVDSSRDRNKP | |
| | GGaCAaACaTGtGTcGTcCAtTAtACa | | FKFMLGKQEVIRGWEEGVAQMS | |
| | GGcATGtTgGAgGAcGGcAAaAAgGT | | VGQRAKLTISPDYAYGATGHPGII | |
| | gGAcagtagtaGaGAtcGcAAtAAaCCtT | | PPHATLVFDVELLKLE | |
| | TcAAaTTcATGtTgGGaAAaCAaGAaG | | | |
| | TcATtaGgGGaTGGGAgGAgGGcGTg | | | |
| | GCtCAaATGtccGTcGGcCAacGcGCt | | | |
| | AAgCTcACcATcagcCCcGAcTAcGCa | | | |
| | TAcGGcGCtACcGGaCAtCCcGGaATt | | | |
| | ATtCCcCCtCAcGCtACctTgGTgTTtG | | | |
| | AcGTcGAaCTgtTgAAgCTcGAa | | | |
| Linker | gtcgag | 1833 | VE | 1834 |
| FKBP_{V} | ggagtgcaggtggagactatctccccaggagac | 1835 | GVQVETISPGDGRTFPKRGQTCV | 1836 |
| | gggcgcaccttccccaagcgcggccagacctgc | | VHYTGMLEDGKKVDSSRDRNKP | |
| | gtggtgcactacaccgggatgcttgaagatggaa | | FKFMLGKQEVIRGWEEGVAQMS | |
| | agaaagttgattcctcccgggacagaaacaagcc | | VGQRAKLTISPDYAYGATGHPGII | |
| | ctttaagtttatgctaggcaagcaggaggtgatccg | | PPHATLVFDVELLKLE | |
| | aggctgggaagaaggggttgcccagatgagtgtg | | | |
| | ggtcagagagccaaactgactatatctccagatta | | | |
| | tgcctatggtgccactgggcacccaggcatcatcc | | | |
| | caccacatgccactctcgtcttcgatgtggagcttct | | | |
| | aaaactggaa | | | |
| STOP | TGA | 1837 | stop | |

APPENDIX 23
pBP1373—pSFG-sp-FKBP.FRB.ΔC9.T2A-αhPSCAscFv.Q.CD8stm.ζ

| | | SEQ | | SEQ |
|---|---|---|---|---|
| Fragment | Nucleotide | ID NO: | Peptide | ID NO: |
| QBI SP163 | AGCGCAGAGGCTTGGGGCAGCCG | 1838 | AQRLGAAERQPGPGPGLGSRRE | 1839 |
| | AGCGGCAGCCAGGCCCCGGCCCG | | RSPPRRARERAASQSEPERERE | |
| | GGCCTCGGTTCCAGAAGGGAGAG | | PRRPRTASET | |
| | GAGCCCGCCAAGGCGCGCAAGAG | | | |
| | AGCGGGCTGCCTCGCAGTCCGAG | | | |
| | CCGGAGAGGGAGCGCGAGCCGCG | | | |
| | CCGGCCCCGGACGGCCTCCGAAA | | | |
| | CC | | | |
| FKBP″ | GGcGTGCAaGTGGAaACTATaAGCC | 1840 | GVQVETISPGDGRTFPKRGQTCV | 1841 |
| | CgGGAGAcGGCcGcACATTtCCCAA | | VHYTGMLEDGKKFDSSRDRNKP | |
| | gAGAGGcCAGACcTGCGTgGTGCAc | | FKFMLGKQEVIRGWEEGVAQMS | |
| | TATACaGGAATGCTGGAgGACGGg | | VGQRAKLTISPDYAYGATGHPGII | |
| | AAGAAaTTCGAtAGCtcCCGGGAtCG | | PPHATLVFDVELLKLE | |
| | AAAtAAGCCtTTCAAaTTCATGCTGG | | | |
| | GcAAGCAaGAAGTcATCaGaGGCTG | | | |
| | GGAaGAAGGcGTCGCcCAGATGTC | | | |
| | cGTGGGtCAGcGcGCCAAgCTGACa | | | |
| | ATTAGtCCAGAtTACGCcTATGGcGC | | | |
| | AACaGGCCAtCCCGGcATCATcCCC | | | |
| | CCaCATGCcACACTcGTCTTtGATGT | | | |
| | cGAGCTcCTGAAaCTGGAg | | | |
| Linker | GGCGGGcaattg | 1842 | ggql | 1843 |
| FRB | gaaatgTGGCATGAAGGGTTGGAAG | 1844 | EMWHEGLEEASRLYFGERNVKG | 1845 |
| | AAGCTTCAAGGCTGTACTTCGGAG | | MFEVLEPLHAMMERGPQTLKETS | |
| | AGAGGAACGTGAAGGGCATGTTTG | | FNQAYGRDLMEAQEWCRKYMK | |
| | AGGTTCTTGAACCTCTGCACGCCA | | SGNVKDLTQAVVDLYYHVFRRISK | |
| | TGATGGAACGGGGACCGCAGACA | | | |
| | CTGAAAGAAACCTCTTTTAATCAGG | | | |
| | CCTACGGCAGAGACCTGATGGAG | | | |
| | GCCCAAGAATGGTGTAGAAAGTAT | | | |
| | ATGAAATCCGGTAACGTGAAAGAC | | | |
| | CTGactCAGGCCTGGGACCTTTATT | | | |
| | ACCATGTGTTCAGGCGGATCAGTA | | | |
| | AG | | | |
| Linker | TCAGGCGGTGGCTCAGGTccatgg | 1846 | SGGGSGPW | 1847 |
| Δcaspase9 | GGATTTGGTGATGTCGGTGCTCTT | 1848 | GFGDVGALESLRGNADLAYILSM | 1849 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 1850 | GSGPR | 1851 |
| T2A | GAAGGCCGAGGGAGCCTGCTGAC | 1852 | EGRGSLLTCGDVEENPGP | 1853 |
| | ATGTGGCGATGTGGAGGAAAACCC | | | |
| | AGGACCA | | | |
| Linker | CCATGG | 1854 | PW | 1855 |
| Signal | ATGGAGTTTGGACTTTCTTGGTTGT | 1856 | MEFGLSVVLFLVAILKGVQCSR | 1857 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 1858 | DIQLTQSPSTLSASMGDRVTITCS | 1859 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 1860 | gggsgggg | 1861 |
| | GC | | | |
| PSCA(A11) | GAGGTGCAGCTCGTGGAGTATGG | 1862 | EVQLVEYGGGLVQPGGSLRLSC | 1863 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 1864 | gs | 1865 |
| CD34 | GAACTTCCTACTCAGGGGACTTTC | 1866 | ELPTQGTFSNVSTNVS | 1867 |
| epitope | TCAAACGTTAGCACAAACGTAAGT | | | |
| CD8 stalk | CCCGCCCCAAGACCCCCCACACCT | 1868 | PAPRPPTPAPTIASQPLSLRPEAC | 1869 |
| | GCGCCGACCATTGCTTCTCAACCC | | RPAAGGAVHTRGLDFACD | |
| | CTGAGTTTGAGACCCGAGGCCTGC | | | |
| | CGGCCAGCTGCCGGCGGGGCCGT | | | |
| | GCATACAAGAGGACTCGATTTCGC | | | |
| | TTGCGAC | | | |
| CD8 | ATCTATATCTGGGCACCTCTCGCT | 1870 | IYIWAPLAGTCGVLLLSLVITLYCN | 1871 |
| trans- | GGCACCTGTGGAGTCCTTCTGCTC | | HRNRRRVCKCPR | |
| membrane | AGCCTGGTTATTACTCTGTACTGTA | | | |
| | ATCACCGGAATCGCCGCCGCGTTT | | | |
| | GTAAGTGTCCCAGG | | | |
| Linker | GTCGAC | 1872 | VD | 1873 |
| CD3ζ | AGAGTGAAGTTCAGCAGGAGCGCA | 1874 | RVKFSRSADAPAYQQGQNQLYN | 1875 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCG | | | |
| STOP | TGA | 1876 | stop | |

APPENDIX 24
pBP1385—pSFG-FRB.FKBP.ΔC9.T2A-ΔCD19

| | | SEQ | | SEQ |
|---|---|---|---|---|
| Fragment | Nucleotide | ID NO: | Peptide | ID NO: |
| FRB | gaaatgTGGCATGAAGGGTTGGAAG | 1877 | EMWHEGLEEASRLYFGERNVKG | 1878 |
| | AAGCTTCAAGGCTGTACTTCGGAG | | MFEVLEPLHAMMERGPQTLKETS | |
| | AGAGGAACGTGAAGGGCATGTTTG | | FNQAYGRDLMEAQEWCRKYMK | |
| | AGGTTCTTGAACCTCTGCACGCCA | | SGNVKDLTQAVVDLYYHVFRRISK | |
| | TGATGGAACGGGGACCGCAGACA | | | |
| | CTGAAAGAAACCTCTTTTAATCAGG | | | |
| | CCTACGGCAGAGACCTGATGGAG | | | |
| | GCCCAAGAATGGTGTAGAAAGTAT | | | |
| | ATGAAATCCGGTAACGTGAAAGAC | | | |
| | CTGactCAGGCCTGGGACCTTTATT | | | |
| | ACCATGTGTTCAGGCGGATCAGTA | | | |
| | AG | | | |
| Linker | GGCGGGcaattg | 1879 | ggql | 1880 |
| FKBP″ | GGcGTGCAaGTGGAaACTATaAGCC | 1881 | GVQVETISPGDGRTFPKRGQTCV | 1882 |
| | CgGGAGAcGGCcGcACATTtCCCAA | | VHYTGMLEDGKKFDSSRDRNKP | |
| | gAGAGGcCAGACcTGCGTgGTGCAc | | FKFMLGKQEVIRGWEEGVAQMS | |
| | TATACaGGAATGCTGGAgGACGGg | | VGQRAKLTISPDYAYGATGHPGII | |
| | AAGAAaTTCGAtAGCtcCCGGGAtCG | | PPHATLVFDVELLKLE | |
| | AAAtAAGCCtTTCAAaTTCATGCTGG | | | |
| | GcAAGCAaGAAGTcATCaGaGGCTG | | | |
| | GGAaGAAGGcGTCGCcCAGATGTC | | | |
| | cGTGGGtCAGcGcGCCAAgCTGACa | | | |
| | ATTAGtCCAGAtTACGCcTATGGcGC | | | |
| | AACaGGCCAtCCCGGcATCATcCCC | | | |
| | CCaCATGCcACACTcGTCTTtGATGT | | | |
| | cGAGCTcCTGAAaCTGGAg | | | |
| Linker | TCAGGCGGTGGCTCAGGTccatgg | 1883 | SGGGSGPW | 1884 |
| Δcaspase9 | GGATTTGGTGATGTCGGTGCTCTT | 1885 | GFGDVGALESLRGNADLAYILSM | 1886 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 1887 | GSGPR | 1888 |
| T2A | GAAGGCCGAGGGAGCCTGCTGAC | 1889 | EGRGSLLTCGDVEENPGP | 1890 |
| | ATGTGGCGATGTGGAGGAAAACCC | | | |
| | AGGACCA | | | |
| ΔCD19 | ATGCCACCACCTCGCCTGCTGTTC | 1891 | MPPPRLLFFLLFLTPMEVRPEEPL | 1892 |
| | TTTCTGCTGTTCCTGACACCTATGG | | VVKVEEGDNAVLQCLKGTSDGPT | |
| | AGGTGCGACCTGAGGAACCACTG | | QQLTWSRESPLKPFLKLSLGLPG | |
| | GTCGTGAAGGTCGAGGAAGGCGA | | LGIHMRPLAIVVLFIFNVSQQMGGF | |
| | CAATGCCGTGCTGCAGTGCCTGAA | | YLCQPGPPSEKAWQPGVVTVNVE | |
| | AGGCACTTCTGATGGGCCAACTCA | | GSGELFRWNVSDLGGLGCGLKN | |
| | GCAGCTGACCTGGTCCAGGGAGT | | RSSEGPSSPSGKLMSPKLYVWA | |
| | CTCCCCTGAAGCCTTTTCTGAAACT | | KDRPEIVVEGEPPCLPPRDSLNQS | |
| | GAGCCTGGGACTGCCAGGACTGG | | LSQDLTMAPGSTLVVLSCGVPPD | |
| | GAATCCACATGCGCCCTCTGGCTA | | SVSRGPLSVVTHVHPKGPKSLLSL | |
| | TCTGGCTGTTCATCTTCAACGTGA | | ELKDDRPARDMVVVMETGLLLPR | |
| | GCCAGCAGATGGGAGGATTCTACC | | ATAQDAGKYYCHRGNLTMSFHL | |
| | TGTGCCAGCCAGGACCACCATCCG | | EITARPVLVVHVVLLRTGGVVKVSA | |
| | AGAAGGCCTGGCAGCCTGGATGG | | VTLAYLIFCLCSLVGILHLQRALVL | |
| | ACCGTCAACGTGGAGGGGTCTGG | | RRKRKRMTDPTRRF | |
| | AGAACTGTTTAGGTGGAATGTGAG | | | |
| | TGACCTGGGAGGACTGGGATGTG | | | |
| | GGCTGAAGAACCGCTCCTCTGAAG | | | |
| | GCCCAAGTTCACCCTCAGGGAAGC | | | |
| | TGATGAGCCCAAAACTGTACGTGT | | | |
| | GGGCCAAAGATCGGCCCGAGATC | | | |
| | TGGGAGGGAGAACCTCCATGCCT | | | |
| | GCCACCTAGAGACAGCCTGAATCA | | | |
| | GAGTCTGTCACAGGATCTGACAAT | | | |
| | GGCCCCCGGGTCCACTCTGTGGC | | | |
| | TGTCTTGTGGAGTCCCACCCGACA | | | |
| | GCGTGTCCAGAGGCCCTCTGTCCT | | | |
| | GGACCCACGTGCATCCTAAGGGG | | | |
| | CCAAAAAGTCTGCTGTCACTGGAA | | | |
| | CTGAAGGACGATCGGCCTGCCAG | | | |
| | AGACATGTGGGTCATGGAGACTGG | | | |
| | ACTGCTGCTGCCACGAGCAACCGC | | | |
| | ACAGGATGCTGGAAAATACTATTG | | | |
| | CCACCGGGGCAATCTGACAATGTC | | | |
| | CTTCCATCTGGAGATCACTGCAAG | | | |
| | GCCCGTGCTGTGGCACTGGCTGC | | | |
| | TGCGAACCGGAGGATGGAAGGTC | | | |
| | AGTGCTGTGACACTGGCATATCTG | | | |
| | ATCTTTTGCCTGTGCTCCCTGGTG | | | |
| | GGCATTCTGCATCTGCAGAGAGCC | | | |
| | CTGGTGCTGCGGAGAAAGAGAAA | | | |
| | GAGAATGACTGACCCAACAAGAAG | | | |
| | GTTT | | | |
| STOP | TGA | 1893 | stop | |

APPENDIX 25
pBP1455—pSFG-MC.FKBP_{wt}.FRB_{L}.T2A-αPSCA.Q.CD8stm.ζ

| | | SEQ | | SEQ |
|---|---|---|---|---|
| Fragment | Nucleotide | ID NO: | Peptide | ID NO: |
| MyD88 | atggctgcaggaggtcccggcgcggggtctgcgg | 1894 | MAAGGPGAGSAAPVSSTSSLPLA | 1895 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 1896 | VE | 1897 |
| CD40 | aaaaaggtggccaagaagccaaccaataaggc | 1898 | KKVAKKPTNKAPHPKQEPQEINF | 1899 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 1900 | VE | 1901 |
| FKBP_{WT}_{^{′}} | GGCGTCCAAGTCGAAACCATTAGT | 1902 | GVQVETISPGDGRTFPKRGQTCV | 1903 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGWEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | gtcgag | 1904 | VE | 1905 |
| FRB_{L} | CAATTGGAAATGTGGCATGAAGGG | 1906 | QLEMWHEGLEEASRLYFGERNV | 1907 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAVVDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| Linker | GGCTCAGGT | 1908 | GSG | 1909 |
| T2A | GAAGGCCGAGGGAGCCTGCTGAC | 1910 | EGRGSLLTCGDVEENPGP | 1911 |
| | ATGTGGCGATGTGGAGGAAAACCC | | | |
| | AGGACCA | | | |
| Linker | CCATGG | 1912 | PW | 1913 |
| Signal | ATGGAGTTTGGACTTTCTTGGTTGT | 1914 | MEFGLSVVLFLVAILKGVQCSR | 1915 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 1916 | DIQLTQSPSTLSASMGDRVTITCS | 1917 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 1918 | gggsgggg | 1919 |
| | GC | | | |
| PSCA(A11) | GAGGTGCAGCTCGTGGAGTATGG | 1920 | EVQLVEYGGGLVQPGGSLRLSC | 1921 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 1922 | gs | 1923 |
| CD34 | GAACTTCCTACTCAGGGGACTTTC | 1924 | ELPTQGTFSNVSTNVS | 1925 |
| epitope | TCAAACGTTAGCACAAACGTAAGT | | | |
| CD8 stalk | CCCGCCCCAAGACCCCCCACACCT | 1926 | PAPRPPTPAPTIASQPLSLRPEAC | 1927 |
| | GCGCCGACCATTGCTTCTCAACCC | | RPAAGGAVHTRGLDFACD | |
| | CTGAGTTTGAGACCCGAGGCCTGC | | | |
| | CGGCCAGCTGCCGGCGGGGCCGT | | | |
| | GCATACAAGAGGACTCGATTTCGC | | | |
| | TTGCGAC | | | |
| CD8 | ATCTATATCTGGGCACCTCTCGCT | 1928 | IYIWAPLAGTCGVLLLSLVITLYCN | 1929 |
| trans- | GGCACCTGTGGAGTCCTTCTGCTC | | HRNRRRVCKCPR | |
| membrane | AGCCTGGTTATTACTCTGTACTGTA | | | |
| | ATCACCGGAATCGCCGCCGCGTTT | | | |
| | GTAAGTGTCCCAGG | | | |
| Linker | GTCGAC | 1930 | VD | 1931 |
| CD3ζ | AGAGTGAAGTTCAGCAGGAGCGCA | 1932 | RVKFSRSADAPAYQQGQNQLYN | 1933 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 26
pBP1466—pSFG-FKBPv.ΔC9.T2A-PSCA.Q.CD8stm.ζ.P2A-MC.FKBP_{wt}.FRB_{L}

| | | SEQ | | SEQ |
|---|---|---|---|---|
| Fragment | Nucleotide | ID NO: | Peptide | ID NO: |
| Leader | ATGCtcgagcaattgGAG | 1934 | MLEQLE | 1935 |
| peptide | | | | |
| FKBPV | GGAGTGCAGGTGGAGACTATTAGC | 1936 | GVQVETISPGDGRTFPKRGQTCV | 1937 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGWEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 1938 | SGGGSGVD | 1939 |
| | C | | | |
| Δcaspase9 | GGATTTGGTGATGTCGGTGCTCTT | 1940 | GFGDVGALESLRGNADLAYILSM | 1941 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 1942 | GSGPR | 1943 |
| T2A | GAAGGCCGAGGGAGCCTGCTGAC | 1944 | EGRGSLLTCGDVEENPGP | 1945 |
| | ATGTGGCGATGTGGAGGAAAACCC | | | |
| | AGGACCA | | | |
| Linker | CCACGG | 1946 | PR | 1947 |
| Signal | ATGGAGTTTGGACTTTCTTGGTTGT | 1948 | MEFGLSVVLFLVAILKGVQCSR | 1949 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 1950 | DIQLTQSPSTLSASMGDRVTITCS | 1951 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 1952 | gggsgggg | 1953 |
| | GC | | | |
| PSCA(A11) | GAGGTGCAGCTCGTGGAGTATGG | 1954 | EVQLVEYGGGLVQPGGSLRLSC | 1955 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 1956 | gs | 1957 |
| CD34 | GAACTTCCTACTCAGGGGACTTTC | 1958 | ELPTQGTFSNVSTNVS | 1959 |
| epitope | TCAAACGTTAGCACAAACGTAAGT | | | |
| CD3ζ | AGAGTGAAGTTCAGCAGGAGCGCA | 1960 | RVKFSRSADAPAYQQGQNQLYN | 1961 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |
| Linker | ggttccgga | 1962 | GSG | 1963 |
| T2A | GAAGGCCGAGGGAGCCTGCTGAC | 1964 | EGRGSLLTCGDVEENPGP | 1965 |
| | ATGTGGCGATGTGGAGGAAAACCC | | | |
| | AGGACCA | | | |
| Linker | ggatctgga | 1966 | GSG | 1967 |
| P2A | GCAACGAATTTTTCCCTGCTGAAA | 1968 | ATNFSLLKQAGDVEENPGP | 1969 |
| | CAGGCAGGGGACGTAGAGGAAAA | | | |
| | TCCTGGTCCT | | | |
| MyD88 | atggctgcaggaggtcccggcgcggggtctgcgg | 1970 | MAAGGPGAGSAAPVSSTSSLPLA | 1971 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 1972 | VE | 1973 |
| CD40 | aaaaaggtggccaagaagccaaccaataaggc | 1974 | KKVAKKPTNKAPHPKQEPQEINF | 1975 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 1976 | VE | 1977 |
| FKBP_{WT}_{^{′}} | GGCGTCCAAGTCGAAACCATTAGT | 1978 | GVQVETISPGDGRTFPKRGQTCV | 1979 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGWEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | gtcgag | 1980 | VE | 1981 |
| FRB_{L} | CAATTGGAAATGTGGCATGAAGGG | 1982 | QLEMWHEGLEEASRLYFGERNV | 1983 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAVVDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| STOPtail | TCAGGCGGTGGCTCAGGTCCGCG | 1984 | SGGGSGPR-stop | 1985 |
| | GTGA | | | |

APPENDIX 27
pBP1474—pSFG-FKBPv.ΔC9.T2A-αHER2.Q.CD8stm.ζ

| | | SEQ | | SEQ ID |
|---|---|---|---|---|
| Fragment | Nucleotide | ID NO: | Peptide | NO: |
| Leader | ATGCtcgagcaattgGAG | 1986 | MLEQLE | 1987 |
| peptide | | | | |
| FKBPv | GGAGTGCAGGTGGAGACTATTAGC | 1988 | GVQVETISPGDGRTFPKRGQTCV | 1989 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 1990 | SGGGSGVD | 1991 |
| | C | | | |
| Δcaspase9 | GGATTTGGTGATGTCGGTGCTCTT | 1992 | GFGDVGALESLRGNADLAYILSM | 1993 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 1994 | GSGPR | 1995 |
| T2A | GAAGGCCGAGGGAGCCTGCTGAC | 1996 | EGRGSLLTCGDVEENPGP | 1997 |
| | ATGTGGCGATGTGGAGGAAAACCC | | | |
| | AGGACCA | | | |
| Linker | GCATGCGCCACC | 1998 | ACAT | 1999 |
| Signal | ATGGAGTTTGGGTTGTCATGGTTG | 2000 | MEFGLSWLFLVAILKGVQCSR | 2001 |
| Peptide | TTTCTCGTCGCTATTCTCAAAGGTG | | | |
| | TACAATGCTCCCGC | | | |
| HER2(FRP | GAAGTCCAATTGCAACAGTCAGGC | 2002 | EVQLQQSGPELKKPGETVKISCK | 2003 |
| 5) VH | CCCGAATTGAAAAAGCCCGGCGAA | | ASGYPFTNYGMNVVVKQAPGQGL | |
| | ACAGTGAAGATATCTTGTAAAGCCT | | KWMGWINTSTGESTFADDFKGR | |
| | CCGGTTACCCTTTTACGAACTATG | | FDFSLETSANTAYLQINNLKSEDM | |
| | GAATGAACTGGGTCAAACAAGCCC | | ATYFCARWEVYHGYVPYWGQGT | |
| | CTGGACAGGGATTGAAGTGGATGG | | TVTVSS | |
| | GATGGATCAATACATCAACAGGCG | | | |
| | AGTCTACCTTCGCAGATGATTTCAA | | | |
| | AGGTCGCTTTGACTTCTCACTGGA | | | |
| | GACCAGTGCAAATACCGCCTACCT | | | |
| | TCAGATTAACAATCTTAAAAGCGAG | | | |
| | GATATGGCAACCTACTTTTGCGCA | | | |
| | AGATGGGAAGTTTATCACGGGTAC | | | |
| | GTGCCATACTGGGGACAAGGAAC | | | |
| | GACAGTGACAGTTAGTAGC | | | |
| Flex | GGCGGTGGAGGCTCCGGTGGAGG | 2004 | GGGGSGGGGSGGGGS | 2005 |
| | CGGCTCTGGAGGAGGAGGTTCA | | | |
| HER2(FRP | GACATCCAATTGACACAATCACAC | 2006 | EVQLVEYGGGLVQPGGSLRLSC | 2007 |
| 5) VL | AAATTTCTCTCAACTTCTGTAGGAG | | AASGFNIKDYYIHVVVRQAPGKGL | |
| | ACAGAGTGAGCATAACCTGCAAAG | | EVVVAWIDPENGDTEFVPKFQGR | |
| | CATCCCAGGACGTGTACAATGCTG | | ATMSADTSKNTAYLQMNSLRAED | |
| | TGGCTTGGTACCAACAGAAGCCTG | | TAVYYCKTGGFWGQGTLVTVSS | |
| | GACAATCCCCAAAATTGCTGATTTA | | | |
| | TTCTGCCTCTAGTAGGTACACTGG | | | |
| | GGTACCTTCTCGGTTTACGGGCTC | | | |
| | TGGGTCCGGACCAGATTTCACGTT | | | |
| | CACAATCAGTTCCGTTCAAGCTGA | | | |
| | AGACCTCGCTGTTTATTTTTGCCAG | | | |
| | CAGCACTTCCGAACCCCTTTTACTT | | | |
| | TTGGCTCAGGCACTAAGTTGGAAA | | | |
| | TCAAGGCTTTG | | | |
| Linker | atgcat | 2008 | MH | 2009 |
| CD34 | GAACTTCCTACTCAGGGGACTTTC | 2010 | ELPTQGTFSNVSTNVS | 2011 |
| epitope | TCAAACGTTAGCACAAACGTAAGT | | | |
| CD3ζ | AGAGTGAAGTTCAGCAGGAGCGCA | 2012 | RVKFSRSADAPAYQQGQNQLYN | 2013 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 28
pBP1475—pSFG-FKBPv.ΔC9.T2A-αPSCA.Q.CD8stm.ζ

| | | | | SEQ |
|---|---|---|---|---|
| | | SEQ | | ID |
| Fragment | Nucleotide | ID NO: | Peptide | NO: |
| Leader | ATGCtcgagcaattgGAG | 2014 | MLEQLE | 2015 |
| peptide | | | | |
| FKBP_{V} | GGAGTGCAGGTGGAGACTATTAGC | 2016 | GVQVETISPGDGRTFPKRGQTCV | 2017 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 2018 | SGGGSGVD | 2019 |
| | C | | | |
| Δcaspase9 | GGATTTGGTGATGTCGGTGCTCTT | 2020 | GFGDVGALESLRGNADLAYILSM | 2021 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 2022 | GSGPR | 2023 |
| T2A | GAAGGCCGAGGGAGCCTGCTGAC | 2024 | EGRGSLLTCGDVEENPGP | 2025 |
| | ATGTGGCGATGTGGAGGAAAACCC | | | |
| | AGGACCA | | | |
| Linker | CCATGG | 2026 | PW | 2027 |
| Signal | ATGGAGTTTGGACTTTCTTGGTTGT | 2028 | MEFGLSWLFLVAILKGVQCSR | 2029 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 2030 | DIQLTQSPSTLSASMGDRVTITCS | 2031 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 2032 | gggsgggg | 2033 |
| | GC | | | |
| PSCA(A11) | GAGGTGCAGCTCGTGGAGTATGG | 2034 | EVQLVEYGGGLVQPGGSLRLSC | 2035 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 2036 | gs | 2037 |
| CD34 | GAACTTCCTACTCAGGGGACTTTC | 2038 | ELPTQGTFSNVSTNVS | 2039 |
| epitope | TCAAACGTTAGCACAAACGTAAGT | | | |
| CD8 stalk | CCCGCCCCAAGACCCCCCACACCT | 2040 | PAPRPPTPAPTIASQPLSLRPEAC | 2041 |
| | GCGCCGACCATTGCTTCTCAACCC | | RPAAGGAVHTRGLDFACD | |
| | CTGAGTTTGAGACCCGAGGCCTGC | | | |
| | CGGCCAGCTGCCGGCGGGGCCGT | | | |
| | GCATACAAGAGGACTCGATTTCGC | | | |
| | TTGCGAC | | | |
| CD8 | ATCTATATCTGGGCACCTCTCGCT | 2042 | IYIWAPLAGTCGVLLLSLVITLYC | 2043 |
| trans- | GGCACCTGTGGAGTCCTTCTGCTC | | NHRNRRRVCKCPR | |
| membrane | AGCCTGGTTATTACTCTGTACTGTA | | | |
| | ATCACCGGAATCGCCGCCGCGTTT | | | |
| | GTAAGTGTCCCAGG | | | |
| Linker | GTCGAC | 2044 | VD | 2045 |
| CD3ζ | AGAGTGAAGTTCAGCAGGAGCGCA | 2046 | RVKFSRSADAPAYQQGQNQLYN | 2047 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 29
pBP1488—pSFG-FRB_{L}.FKBP_{wt}.MC-T2A-αPSCA.Q.CD8stm.ζ

| | | SEQ | | SEQ ID |
|---|---|---|---|---|
| Fragment | Nucleotide | ID NO: | Peptide | NO: |
| FRB_{L} | ATGCAATTGGAAATGTGGCATGAA | 2048 | MQLEMWHEGLEEASRLYFGERN | 2049 |
| | GGGTTGGAAGAAGCTTCAAGGCTG | | VKGMFEVLEPLHAMMERGPQTL | |
| | TACTTCGGAGAGAGGAACGTGAAG | | KETSFNQAYGRDLMEAQEWCRK | |
| | GGCATGTTTGAGGTTCTTGAACCT | | YMKSGNVKDLLQAWDLYYHVFR | |
| | CTGCACGCCATGATGGAACGGGG | | RISK | |
| | ACCGCAGACACTGAAAGAAACCTC | | | |
| | TTTTAATCAGGCCTACGGCAGAGA | | | |
| | CCTGATGGAGGCCCAAGAATGGTG | | | |
| | TAGAAAGTATATGAAATCCGGTAAC | | | |
| | GTGAAAGACCTGCTCCAGGCCTGG | | | |
| | GACCTTTATTACCATGTGTTCAGGC | | | |
| | GGATCAGTAAG | | | |
| Linker | TCAGGCGGTGGCAGCGGCCAATT | 2050 | sgggsgql | 2051 |
| | G | | | |
| FKBP_{WT}^{′} | GGaGTCCAAGTCGAAACCATTAGT | 2052 | GVQVETISPGDGRTFPKRGQTCV | 2053 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGVVEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | GGAAGCATGCGGATCGGA | 2054 | gsmrig | 2055 |
| MyD88 | atggctgcaggaggtcccggcgcggggtctgcgg | 2056 | MAAGGPGAGSAAPVSSTSSLPLA | 2057 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 2058 | VE | 2059 |
| CD40 | aaaaaggtggccaagaagccaaccaataaggc | 2060 | KKVAKKPTNKAPHPKQEPQEINF | 2061 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | GGCAGTGGGCCGCGG | 2062 | gsgpr | 2063 |
| T2A | GAAGGCCGAGGGAGCCTGCTGAC | 2064 | EGRGSLLTCGDVEENPGP | 2065 |
| | ATGTGGCGATGTGGAGGAAAACCC | | | |
| | AGGACCA | | | |
| Linker | CCATGG | 2066 | PW | 2067 |
| Signal | ATGGAGTTTGGACTTTCTTGGTTGT | 2068 | MEFGLSWLFLVAILKGVQCSR | 2069 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 2070 | DIQLTQSPSTLSASMGDRVTITCS | 2071 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 2072 | gggsgggg | 2073 |
| | GC | | | |
| PSCA(A11) | GAGGTGCAGCTCGTGGAGTATGG | 2074 | EVQLVEYGGGLVQPGGSLRLSC | 2075 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 2076 | gs | 2077 |
| CD34 | GAACTTCCTACTCAGGGGACTTTC | 2078 | ELPTQGTFSNVSTNVS | 2079 |
| epitope | TCAAACGTTAGCACAAACGTAAGT | | | |
| CD8 stalk | CCCGCCCCAAGACCCCCCACACCT | 2080 | PAPRPPTPAPTIASQPLSLRPEAC | 2081 |
| | GCGCCGACCATTGCTTCTCAACCC | | RPAAGGAVHTRGLDFACD | |
| | CTGAGTTTGAGACCCGAGGCCTGC | | | |
| | CGGCCAGCTGCCGGCGGGGCCGT | | | |
| | GCATACAAGAGGACTCGATTTCGC | | | |
| | TTGCGAC | | | |
| CD8 | ATCTATATCTGGGCACCTCTCGCT | 2082 | IYIWAPLAGTCGVLLLSLVITLYCN | 2083 |
| trans- | GGCACCTGTGGAGTCCTTCTGCTC | | HRNRRRVCKCPR | |
| membrane | AGCCTGGTTATTACTCTGTACTGTA | | | |
| | ATCACCGGAATCGCCGCCGCGTTT | | | |
| | GTAAGTGTCCCAGG | | | |
| Linker | GTCGAC | 2084 | VD | 2085 |
| CD3ζ | AGAGTGAAGTTCAGCAGGAGCGCA | 2086 | RVKFSRSADAPAYQQGQNQLYN | 2087 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 30
pBP1491—pSFG—FKBPv.ΔC9.P2A.MC.FKBP_{wt}.FRB_{L}.T2A-αHER2.Q.CD8stm.ζ

| | | SEQ | | SEQ ID |
|---|---|---|---|---|
| Fragment | Nucleotide | ID NO: | Peptide | NO: |
| Linker | atgcatATGCTGGAG | 2088 | MHMLE | 2089 |
| FKBP_{V} | GGAGTGCAGGTGGAGACTATTAGC | 2090 | GVQVETISPGDGRTFPKRGQTCV | 2091 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 2092 | SGGGSGVD | 2093 |
| | C | | | |
| Δcaspase9 | GGATTTGGTGATGTCGGTGCTCTT | 2094 | GFGDVGALESLRGNADLAYILSM | 2095 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | agcggCCGCaggtagcggg | 2096 | aaaGSG | 2097 |
| MyD88 | atggctgcaggaggtcccggcgcggggtctgcgg | 2098 | MAAGGPGAGSAAPVSSTSSLPLA | 2099 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 2100 | VE | 2101 |
| CD40 | aaaaaggtggccaagaagccaaccaataaggc | 2102 | KKVAKKPTNKAPHPKQEPQEINF | 2103 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gcggCCGCaggtagcggg | 2104 | aaaGSG | 2105 |
| P2A | GCAACGAATTTTTCCCTGCTGAAA | 2106 | ATNFSLLKQAGDVEENPGP | 2107 |
| | CAGGCAGGGGACGTAGAGGAAAA | | | |
| | TCCTGGTCCT | | | |
| Linker | gtcgag | 2108 | VE | 2109 |
| FKBP_{WT}_{^{'}} | GGCGTCCAAGTCGAAACCATTAGT | 2110 | GVQVETISPGDGRTFPKRGQTCV | 2111 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGVVEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | gtcgag | 2112 | VE | 2113 |
| FRB_{L} | CAATTGGAAATGTGGCATGAAGGG | 2114 | QLEMWHEGLEEASRLYFGERNV | 2115 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAWDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| Linker | ggatctggaccgcgg | 2118 | GSGpr | 2119 |
| T2A | GAAGGCCGAGGGAGCCTGCTGAC | 2120 | EGRGSLLTCGDVEENPGP | 2121 |
| | ATGTGGCGATGTGGAGGAAAACCC | | | |
| | AGGACCA | | | |
| Linker | GCATGCGCCACC | 2122 | ACAT | 2123 |
| Signal | ATGGAGTTTGGGTTGTCATGGTTG | 2124 | MEFGLSWLFLVAILKGVQCSR | 2125 |
| Peptide | TTTCTCGTCGCTATTCTCAAAGGTG | | | |
| | TACAATGCTCCCGC | | | |
| HER2(FRP | GAAGTCCAATTGCAACAGTCAGGC | 2126 | EVQLQQSGPELKKPGETVKISCK | 2127 |
| 5)VH | CCCGAATTGAAAAAGCCCGGCGAA | | ASGYPFTNYGMNVVVKQAPGQGL | |
| | ACAGTGAAGATATCTTGTAAAGCCT | | KWMGWINTSTGESTFADDFKGR | |
| | CCGGTTACCCTTTTACGAACTATG | | FDFSLETSANTAYLQINNLKSEDM | |
| | GAATGAACTGGGTCAAACAAGCCC | | ATYFCARWEVYHGYVPYWGQGT | |
| | CTGGACAGGGATTGAAGTGGATGG | | TVTVSS | |
| | GATGGATCAATACATCAACAGGCG | | | |
| | AGTCTACCTTCGCAGATGATTTCAA | | | |
| | AGGTCGCTTTGACTTCTCACTGGA | | | |
| | GACCAGTGCAAATACCGCCTACCT | | | |
| | TCAGATTAACAATCTTAAAAGCGAG | | | |
| | GATATGGCAACCTACTTTTGCGCA | | | |
| | AGATGGGAAGTTTATCACGGGTAC | | | |
| | GTGCCATACTGGGGACAAGGAAC | | | |
| | GACAGTGACAGTTAGTAGC | | | |
| Flex | GGCGGTGGAGGCTCCGGTGGAGG | 2128 | GGGGSGGGGSGGGGS | 2129 |
| | CGGCTCTGGAGGAGGAGGTTCA | | | |
| HER2(FRP | GACATCCAATTGACACAATCACAC | 2130 | EVQLVEYGGGLVQPGGSLRLSC | 2131 |
| 5)VL | AAATTTCTCTCAACTTCTGTAGGAG | | AASGFNIKDYYIHVVVRQAPGKGL | |
| | ACAGAGTGAGCATAACCTGCAAAG | | EVVVAWIDPENGDTEFVPKFQGR | |
| | CATCCCAGGACGTGTACAATGCTG | | ATMSADTSKNTAYLQMNSLRAED | |
| | TGGCTTGGTACCAACAGAAGCCTG | | TAVYYCKTGGFWGQGTLVTVSS | |
| | GACAATCCCCAAAATTGCTGATTTA | | | |
| | TTCTGCCTCTAGTAGGTACACTGG | | | |
| | GGTACCTTCTCGGTTTACGGGCTC | | | |
| | TGGGTCCGGACCAGATTTCACGTT | | | |
| | CACAATCAGTTCCGTTCAAGCTGA | | | |
| | AGACCTCGCTGTTTATTTTTGCCAG | | | |
| | CAGCACTTCCGAACCCCTTTTACTT | | | |
| | TTGGCTCAGGCACTAAGTTGGAAA | | | |
| | TCAAGGCTTTG | | | |
| Linker | atgcat | 2132 | MH | 2133 |
| CD34 | GAACTTCCTACTCAGGGGACTTTC | 2134 | ELPTQGTFSNVSTNVS | 2135 |
| epitope | TCAAACGTTAGCACAAACGTAAGT | | | |
| CD3ζ | AGAGTGAAGTTCAGCAGGAGCGCA | 2136 | RVKFSRSADAPAYQQGQNQLYN | 2137 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 31
pBP1493—pSFG-MC.FKBP_{wt}.FRB_{L}-P2A.FKBPv.ΔC9.T2A-αHER2.Q.CD8stm.ζ

| | | SEQ | | SEQ ID |
|---|---|---|---|---|
| Fragment | Nucleotide | ID NO: | Peptide | NO: |
| MyD88 | atggctgcaggaggtcccggcgcggggtctgcgg | 2138 | MAAGGPGAGSAAPVSSTSSLPLA | 2139 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 2140 | VE | 2141 |
| CD40 | aaaaaggtggccaagaagccaaccaataaggc | 2142 | KKVAKKPTNKAPHPKQEPQEINF | 2143 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 2144 | VE | 2145 |
| FKBP_{WT}_{^{'}} | GGCGTCCAAGTCGAAACCATTAGT | 2146 | GVQVETISPGDGRTFPKRGQTCV | 2147 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGVVEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | gtcgag | 2148 | VE | 2149 |
| FRB_{L} | CAATTGGAAATGTGGCATGAAGGG | 2150 | QLEMWHEGLEEASRLYFGERNV | 2151 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAWDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| Linker | gcggCCGCaggtagcggg | 2152 | aaaGSG | 2153 |
| P2A | GCAACGAATTTTTCCCTGCTGAAA | 2154 | ATNFSLLKQAGDVEENPGP | 2155 |
| | CAGGCAGGGGACGTAGAGGAAAA | | | |
| | TCCTGGTCCT | | | |
| Linker | ggatctgga | 2156 | GSG | 2157 |
| FKBPv | GGAGTGCAGGTGGAGACTATTAGC | 2158 | GVQVETISPGDGRTFPKRGQTCV | 2159 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 2160 | SGGGSGVD | 2161 |
| | C | | | |
| Δcaspase9 | GGATTTGGTGATGTCGGTGCTCTT | 2162 | GFGDVGALESLRGNADLAYILSM | 2163 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 2164 | GSGPR | 2165 |
| T2A | GAAGGCCGAGGGAGCCTGCTGAC | 2166 | EGRGSLLTCGDVEENPGP | 2167 |
| | ATGTGGCGATGTGGAGGAAAACCC | | | |
| | AGGACCA | | | |
| Linker | GCATGCGCCACC | 2168 | ACAT | 2169 |
| Signal | ATGGAGTTTGGGTTGTCATGGTTG | 2170 | MEFGLSWLFLVAILKGVQCSR | 2171 |
| Peptide | TTTCTCGTCGCTATTCTCAAAGGTG | | | |
| | TACAATGCTCCCGC | | | |
| HER2(FRP | GAAGTCCAATTGCAACAGTCAGGC | 2172 | EVQLQQSGPELKKPGETVKISCK | 2173 |
| 5)VH | CCCGAATTGAAAAAGCCCGGCGAA | | ASGYPFTNYGMNVVVKQAPGQGL | |
| | ACAGTGAAGATATCTTGTAAAGCCT | | KWMGWINTSTGESTFADDFKGR | |
| | CCGGTTACCCTTTTACGAACTATG | | FDFSLETSANTAYLQINNLKSEDM | |
| | GAATGAACTGGGTCAAACAAGCCC | | ATYFCARWEVYHGYVPYWGQGT | |
| | CTGGACAGGGATTGAAGTGGATGG | | TVTVSS | |
| | GATGGATCAATACATCAACAGGCG | | | |
| | AGTCTACCTTCGCAGATGATTTCAA | | | |
| | AGGTCGCTTTGACTTCTCACTGGA | | | |
| | GACCAGTGCAAATACCGCCTACCT | | | |
| | TCAGATTAACAATCTTAAAAGCGAG | | | |
| | GATATGGCAACCTACTTTTGCGCA | | | |
| | AGATGGGAAGTTTATCACGGGTAC | | | |
| | GTGCCATACTGGGGACAAGGAAC | | | |
| | GACAGTGACAGTTAGTAGC | | | |
| Flex | GGCGGTGGAGGCTCCGGTGGAGG | 2174 | GGGGSGGGGSGGGGS | 2175 |
| | CGGCTCTGGAGGAGGAGGTTCA | | | |
| HER2(FRP | GACATCCAATTGACACAATCACAC | 2176 | EVQLVEYGGGLVQPGGSLRLSC | 2177 |
| 5)VL | AAATTTCTCTCAACTTCTGTAGGAG | | AASGFNIKDYYIHVVVRQAPGKGL | |
| | ACAGAGTGAGCATAACCTGCAAAG | | EVVVAWIDPENGDTEFVPKFQGR | |
| | CATCCCAGGACGTGTACAATGCTG | | ATMSADTSKNTAYLQMNSLRAED | |
| | TGGCTTGGTACCAACAGAAGCCTG | | TAVYYCKTGGFWGQGTLVTVSS | |
| | GACAATCCCCAAAATTGCTGATTTA | | | |
| | TTCTGCCTCTAGTAGGTACACTGG | | | |
| | GGTACCTTCTCGGTTTACGGGCTC | | | |
| | TGGGTCCGGACCAGATTTCACGTT | | | |
| | CACAATCAGTTCCGTTCAAGCTGA | | | |
| | AGACCTCGCTGTTTATTTTTGCCAG | | | |
| | CAGCACTTCCGAACCCCTTTTACTT | | | |
| | TTGGCTCAGGCACTAAGTTGGAAA | | | |
| | TCAAGGCTTTG | | | |
| Linker | atgcat | 2178 | MH | 2179 |
| CD34 | GAACTTCCTACTCAGGGGACTTTC | 2180 | ELPTQGTFSNVSTNVS | 2181 |
| epitope | TCAAACGTTAGCACAAACGTAAGT | | | |
| CD3ζ | AGAGTGAAGTTCAGCAGGAGCGCA | 2182 | RVKFSRSADAPAYQQGQNQLYN | 2183 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 32
pBP1494—pSFG-MC.FKBP_{wt}.FRB_{L}-P2A.FKBPv.ΔC9.T2A-PSCA.Q.CD8stm.ζ

| | | SEQ | | SEQ ID |
|---|---|---|---|---|
| Fragment | Nucleotide | ID NO: | Peptide | NO: |
| MyD88 | atggctgcaggaggtcccggcgcggggtctgcgg | 2184 | MAAGGPGAGSAAPVSSTSSLPLA | 2185 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 2186 | VE | 2187 |
| CD40 | aaaaaggtggccaagaagccaaccaataaggc | 2188 | KKVAKKPTNKAPHPKQEPQEINF | 2189 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | gtcgag | 2190 | VE | 2191 |
| FKBP_{WT}_{^{'}} | GGCGTCCAAGTCGAAACCATTAGT | 2192 | GVQVETISPGDGRTFPKRGQTCV | 2193 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGVVEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| Linker | gtcgag | 2194 | VE | 2195 |
| FRB_{L} | CAATTGGAAATGTGGCATGAAGGG | 2196 | QLEMWHEGLEEASRLYFGERNV | 2197 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAWDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| Linker | gcggCCGCaggtagcggg | 2198 | aaaGSG | 2199 |
| P2A | GCAACGAATTTTTCCCTGCTGAAA | 2200 | ATNFSLLKQAGDVEENPGP | 2201 |
| | CAGGCAGGGGACGTAGAGGAAAA | | | |
| | TCCTGGTCCT | | | |
| Linker | atgcatATGCTGGAG | 2202 | MHMLE | 2203 |
| FKBPv | GGAGTGCAGGTGGAGACTATTAGC | 2204 | GVQVETISPGDGRTFPKRGQTCV | 2205 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 2206 | SGGGSGVD | 2207 |
| | C | | | |
| Δcaspase9 | GGATTTGGTGATGTCGGTGCTCTT | 2208 | GFGDVGALESLRGNADLAYILSM | 2209 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 2210 | GSGPR | 2211 |
| T2A | GAAGGCCGAGGGAGCCTGCTGAC | 2212 | EGRGSLLTCGDVEENPGP | 2213 |
| | ATGTGGCGATGTGGAGGAAAACCC | | | |
| | AGGACCA | | | |
| Linker | CCACGG | 2214 | PR | 2215 |
| Signal | ATGGAGTTTGGACTTTCTTGGTTGT | 2216 | MEFGLSWLFLVAILKGVQCSR | 2217 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 2218 | DIQLTQSPSTLSASMGDRVTITCS | 2219 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVE1K | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 2220 | gggsgggg | 2221 |
| | GC | | | |
| PSCA(A11) | GAGGTGCAGCTCGTGGAGTATGG | 2222 | EVQLVEYGGGLVQPGGSLRLSC | 2223 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 2224 | gs | 2225 |
| CD34 | GAACTTCCTACTCAGGGGACTTTC | 2226 | ELPTQGTFSNVSTNVS | 2227 |
| epitope | TCAAACGTTAGCACAAACGTAAGT | | | |
| CD3ζ | AGAGTGAAGTTCAGCAGGAGCGCA | 2228 | RVKFSRSADAPAYQQGQNQLYN | 2229 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 33
pBP1757—pSFG-FRB_{L}.FKBP_{wt}.MC-P2A.FKBPv.ΔC9.T2A-αPSCA.Q.CD8stm.ζ

| | | SEQ | | SEQ ID |
|---|---|---|---|---|
| Fragment | Nucleotide | ID NO: | Peptide | NO: |
| FRB_{L} | ATGTTGGAAATGTGGCATGAAGGG | 2230 | MLEMWHEGLEEASRLYFGERNV | 2231 |
| | TTGGAAGAAGCTTCAAGGCTGTAC | | KGMFEVLEPLHAMMERGPQTLK | |
| | TTCGGAGAGAGGAACGTGAAGGG | | ETSFNQAYGRDLMEAQEWCRKY | |
| | CATGTTTGAGGTTCTTGAACCTCTG | | MKSGNVKDLLQAWDLYYHVFRRI | |
| | CACGCCATGATGGAACGGGGACC | | SK | |
| | GCAGACACTGAAAGAAACCTCTTT | | | |
| | TAATCAGGCCTACGGCAGAGACCT | | | |
| | GATGGAGGCCCAAGAATGGTGTAG | | | |
| | AAAGTATATGAAATCCGGTAACGT | | | |
| | GAAAGACCTGCTCCAGGCCTGGG | | | |
| | ACCTTTATTACCATGTGTTCAGGCG | | | |
| | GATCAGTAAG | | | |
| Linker | gtcgag | 2232 | VE | 2233 |
| FKBP_{WT}_{^{′}} | GGCGTCCAAGTCGAAACCATTAGT | 2234 | GVQVETISPGDGRTFPKRGQTCV | 2235 |
| | CCCGGCGATGGCAGAACATTTCCT | | VHYTGMLEDGKKFDSSRDRNKP | |
| | ACAAGGGGACAAACATGTGTCGTC | | FKFMLGKQEVIRGVVEEGVAQMS | |
| | CATTATACAGGCATGTTGGAGGAC | | VGQRAKLTISPDYAYGATGHPGII | |
| | GGCAAAAAGTTCGACAGTAGTAGA | | PPHATLVFDVELLKLE | |
| | GATCGCAATAAACCTTTCAAATTCA | | | |
| | TGTTGGGAAAACAAGAAGTCATTA | | | |
| | GGGGATGGGAGGAGGGCGTGGCT | | | |
| | CAAATGTCCGTCGGCCAACGCGCT | | | |
| | AAGCTCACCATCAGCCCCGACTAC | | | |
| | GCATACGGCGCTACCGGACATCCC | | | |
| | GGAATTATTCCCCCTCACGCTACC | | | |
| | TTGGTGTTTGACGTCGAACTGTTG | | | |
| | AAGCTCGAA | | | |
| MyD88 | atggctgcaggaggtcccggcgcggggtctgcgg | 2236 | MAAGGPGAGSAAPVSSTSSLPLA | 2237 |
| | ccccggtctcctccacatcctcccttcccctggctgc | | ALNMRVRRRLSLFLNVRTQVAAD | |
| | tctcaacatgcgagtgcggcgccgcctgtctctgtt | | WTALAEEMDFEYLEIRQLETQAD | |
| | cttgaacgtgcggacacaggtggcggccgactgg | | PTGRLLDAWQGRPGASVGRLLD | |
| | accgcgctggcggaggagatggactttgagtactt | | LLTKLGRDDVLLELGPSIEEDCQK | |
| | ggagatccggcaactggagacacaagcggacc | | YILKQQQEEAEKPLQVAAVDSSV | |
| | ccactggcaggctgctggacgcctggcagggac | | PRTAELAGITTLDDPLGHMPERF | |
| | gccctggcgcctctgtaggccgactgctcgatctgc | | DAFICYCPSDI | |
| | ttaccaagctgggccgcgacgacgtgctgctgga | | | |
| | gctgggacccagcattgaggaggattgccaaaa | | | |
| | gtatatcttgaagcagcagcaggaggaggctgag | | | |
| | aagcctttacaggtggccgctgtagacagcagtgt | | | |
| | cccacggacagcagagctggcgggcatcacca | | | |
| | cacttgatgaccccctggggcatatgcctgagcgtt | | | |
| | tcgatgccttcatctgctattgccccagcgacatc | | | |
| Linker | gtcgag | 2238 | VE | 2239 |
| CD40 | aaaaaggtggccaagaagccaaccaataaggc | 2240 | KKVAKKPTNKAPHPKQEPQEINF | 2241 |
| | cccccaccccaagcaggagccccaggagatca | | PDDLPGSNTAAPVQETLHGCQP | |
| | attttcccgacgatcttcctggctccaacactgctgct | | VTQEDGKESRISVQERQ | |
| | ccagtgcaggagactttacatggatgccaaccggt | | | |
| | cacccaggaggatggcaaagagagtcgcatctc | | | |
| | agtgcaggagagacag | | | |
| Linker | ggatctgga | 2242 | GSG | 2243 |
| P2A | GCAACGAATTTTTCCCTGCTGAAA | 2244 | ATNFSLLKQAGDVEENPGP | 2245 |
| | CAGGCAGGGGACGTAGAGGAAAA | | | |
| | TCCTGGTCCT | | | |
| Linker | atgcatATGCTGGAG | 2246 | MHMLE | 2247 |
| FKBPv | GGAGTGCAGGTGGAGACTATTAGC | 2248 | GVQVETISPGDGRTFPKRGQTCV | 2249 |
| | CCCGGAGATGGCAGAACATTCCCC | | VHYTGMLEDGKKVDSSRDRNKP | |
| | AAAAGAGGACAGACTTGCGTCGTG | | FKFMLGKQEVIRGVVEEGVAQMS | |
| | CATTATACTGGAATGCTGGAAGAC | | VGQRAKLTISPDYAYGATGHPGII | |
| | GGCAAGAAGGTGGACAGCAGCCG | | PPHATLVFDVELLKLE | |
| | GGACCGAAACAAGCCCTTCAAGTT | | | |
| | CATGCTGGGGAAGCAGGAAGTGAT | | | |
| | CCGGGGCTGGGAGGAAGGAGTCG | | | |
| | CACAGATGTCAGTGGGACAGAGG | | | |
| | GCCAAACTGACTATTAGCCCAGAC | | | |
| | TACGCTTATGGAGCAACCGGCCAC | | | |
| | CCCGGGATCATTCCCCCTCATGCT | | | |
| | ACACTGGTCTTCGATGTGGAGCTG | | | |
| | CTGAAGCTGGAA | | | |
| Linker | TCAGGCGGTGGCTCAGGTGTGGA | 2250 | SGGGSGVD | 2251 |
| | C | | | |
| Δcaspase9 | GGATTTGGTGATGTCGGTGCTCTT | 2252 | GFGDVGALESLRGNADLAYILSM | 2253 |
| | GAGAGTTTGAGGGGAAATGCAGAT | | EPCGHCLIINNVNFCRESGLRTRT | |
| | TTGGCTTACATCCTGAGCATGGAG | | GSNIDCEKLRRRFSSLHFMVEVK | |
| | CCCTGTGGCCACTGCCTCATTATC | | GDLTAKKMVLALLELARQDHGAL | |
| | AACAATGTGAACTTCTGCCGTGAG | | DCCVVVILSHGCQASHLQFPGAV | |
| | TCCGGGCTCCGCACCCGCACTGG | | YGTDGCPVSVEKIVNIFNGTSCPS | |
| | CTCCAACATCGACTGTGAGAAGTT | | LGGKPKLFFIQACGGEQKDHGFE | |
| | GCGGCGTCGCTTCTCCTCGCTGCA | | VASTSPEDESPGSNPEPDATPFQ | |
| | TTTCATGGTGGAGGTGAAGGGCGA | | EGLRTFDQLDAISSLPTPSDIFVS | |
| | CCTGACTGCCAAGAAAATGGTGCT | | YSTFPGFVSVVRDPKSGSVVYVET | |
| | GGCTTTGCTGGAGCTGGCGCgGCA | | LDDIFEQWAHSEDLQSLLLRVAN | |
| | GGACCACGGTGCTCTGGACTGCT | | AVSVKGIYKQMPGCFNFLRKKLF | |
| | GCGTGGTGGTCATTCTCTCTCACG | | FKTSASRA | |
| | GCTGTCAGGCCAGCCACCTGCAGT | | | |
| | TCCCAGGGGCTGTCTACGGCACA | | | |
| | GATGGATGCCCTGTGTCGGTCGAG | | | |
| | AAGATTGTGAACATCTTCAATGGG | | | |
| | ACCAGCTGCCCCAGCCTGGGAGG | | | |
| | GAAGCCCAAGCTCTTTTTCATCCA | | | |
| | GGCCTGTGGTGGGGAGCAGAAAG | | | |
| | AtCATGGGTTTGAGGTGGCCTCCA | | | |
| | CTTCCCCTGAAGACGAGTCCCCTG | | | |
| | GCAGTAACCCCGAGCCAGATGCCA | | | |
| | CCCCGTTCCAGGAAGGTTTGAGGA | | | |
| | CCTTCGACCAGCTGGACGCCATAT | | | |
| | CTAGTTTGCCCACACCCAGTGACA | | | |
| | TCTTTGTGTCCTACTCTACTTTCCC | | | |
| | AGGTTTTGTTTCCTGGAGGGACCC | | | |
| | CAAGAGTGGCTCCTGGTACGTTGA | | | |
| | GACCCTGGACGACATCTTTGAGCA | | | |
| | GTGGGCTCACTCTGAAGACCTGCA | | | |
| | GTCCCTCCTGCTTAGGGTCGCTAA | | | |
| | TGCTGTTTCGGTGAAAGGGATTTA | | | |
| | TAAACAGATGCCTGGTTGCTTTAAT | | | |
| | TTCCTCCGGAAAAAACTTTTCTTTA | | | |
| | AAACATCAGCTAGCAGAGCC | | | |
| Linker | ggatctggaccgcGG | 2254 | GSGPR | 2255 |
| T2A | GAAGGCCGAGGGAGCCTGCTGAC | 2256 | EGRGSLLTCGDVEENPGP | 2257 |
| | ATGTGGCGATGTGGAGGAAAACCC | | | |
| | AGGACCA | | | |
| Linker | CCACGG | 2258 | PR | 2259 |
| Signal | ATGGAGTTTGGACTTTCTTGGTTGT | 2260 | MEFGLSWLFLVAILKGVQCSR | 2261 |
| Peptide | TTTTGGTGGCAATTCTGAAGGGTG | | | |
| | TCCAGTGTAGCAGG | | | |
| PSCA(A11) | GACATCCAACTGACGCAAAGCCCA | 2262 | DIQLTQSPSTLSASMGDRVTITCS | 2263 |
| VL | TCTACACTCAGCGCTAGCATGGGG | | ASSSVRFIHVVYQQKPGKAPKRLI | |
| | GACAGGGTCACAATCACGTGCTCT | | YDTSKLASGVPSRFSGSGSGTDF | |
| | GCCTCAAGTTCCGTTAGGTTTATC | | TLTISSLQPEDFATYYCQQWGSS | |
| | CATTGGTATCAGCAGAAACCTGGA | | PFTFGQGTKVEIK | |
| | AAGGCCCCAAAAAGACTGATCTAT | | | |
| | GATACCAGCAAGCTGGCTTCCGGA | | | |
| | GTGCCCTCAAGGTTCTCAGGATCT | | | |
| | GGCAGTGGGACCGATTTCACCCTG | | | |
| | ACAATTAGCAGCCTTCAGCCAGAG | | | |
| | GATTTCGCAACCTATTACTGTCAGC | | | |
| | AATGGGGGTCCAGCCCATTCACTT | | | |
| | TCGGCCAAGGAACAAAGGTGGAG | | | |
| | ATAAAA | | | |
| Flex | GGCGGAGGAAGCGGAGGTGGGG | 2264 | gggsgggg | 2265 |
| | GC | | | |
| PSCA(A11) | GAGGTGCAGCTCGTGGAGTATGG | 2266 | EVQLVEYGGGLVQPGGSLRLSC | 2267 |
| VH | CGGGGGCCTGGTGCAGCCTGGGG | | AASGFNIKDYYIHVVVRQAPGKGL | |
| | GTAGTCTGAGGCTCTCCTGCGCTG | | EVVVAWIDPENGDTEFVPKFQGR | |
| | CCTCTGGCTTTAACATTAAAGACTA | | ATMSADTSKNTAYLQMNSLRAED | |
| | CTACATACATTGGGTGCGGCAGGC | | TAVYYCKTGGFWGQGTLVTVSS | |
| | CCCAGGCAAAGGGCTCGAATGGG | | | |
| | TGGCCTGGATTGACCCTGAGAATG | | | |
| | GTGACACTGAGTTTGTCCCCAAGT | | | |
| | TTCAGGGCAGAGCCACCATGAGC | | | |
| | GCTGACACAAGCAAAAACACTGCT | | | |
| | TATCTCCAAATGAATAGCCTGCGA | | | |
| | GCTGAAGATACAGCAGTCTATTAC | | | |
| | TGCAAGACGGGAGGATTCTGGGG | | | |
| | CCAGGGAACTCTGGTGACAGTTAG | | | |
| | TTCC | | | |
| Linker | GGATCC | 2268 | gs | 2269 |
| CD34 | GAACTTCCTACTCAGGGGACTTTC | 2270 | ELPTQGTFSNVSTNVS | 2271 |
| epitope | TCAAACGTTAGCACAAACGTAAGT | | | |
| CD3ζ | AGAGTGAAGTTCAGCAGGAGCGCA | 2272 | RVKFSRSADAPAYQQGQNQLYN | 2273 |
| | GACGCCCCCGCGTACCAGCAGGG | | ELNLGRREEYDVLDKRRGRDPE | |
| | CCAGAACCAGCTCTATAACGAGCT | | MGGKPRRKNPQEGLYNELQKDK | |
| | CAATCTAGGACGAAGAGAGGAGTA | | MAEAYSEIGMKGERRRGKGHDG | |
| | CGATGTTTTGGACAAGAGACGTGG | | LYQGLSTATKDTYDALHMQALPP | |
| | CCGGGACCCTGAGATGGGGGGAA | | R | |
| | AGCCGAGAAGGAAGAACCCTCAG | | | |
| | GAAGGCCTGTACAATGAACTGCAG | | | |
| | AAAGATAAGATGGCGGAGGCCTAC | | | |
| | AGTGAGATTGGGATGAAAGGCGAG | | | |
| | CGCCGGAGGGGCAAGGGGCACGA | | | |
| | TGGCCTTTACCAGGGTCTCAGTAC | | | |
| | AGCCACCAAGGACACCTACGACGC | | | |
| | CCTTCACATGCAAGCTCTTCCACC | | | |
| | TCGT | | | |

APPENDIX 34
pBP1759—pSFG—FRB_{L}.FKBP_{wt}.MC-P2A.FKBPv.ΔC9.T2A-αHER2.Q.CD8stm.ζ

| | | Seq ID | | Seq ID |
|---|---|---|---|---|
| Fragment | Nucleotide | No: | Peptide | No: |
| FRB_{L} | ATGTTGGAAATGTGGCATGAAGGGTTG | 2274 | MLEMWHEGLEEASRLYFGER | 2275 |
| | GAAGAAGCTTCAAGGCTGTACTTCGGA | | NVKGMFEVLEPLHAMMERGP | |
| | GAGAGGAACGTGAAGGGCATGTTTGAG | | QTLKETSFNQAYGRDLMEAQ | |
| | GTTCTTGAACCTCTGCACGCCATGATGG | | EWCRKYMKSGNVKDLLQAW | |
| | AACGGGGACCGCAGACACTGAAAGAAA | | DLYYHVFRRISK | |
| | CCTCTTTTAATCAGGCCTACGGCAGAGA | | | |
| | CCTGATGGAGGCCCAAGAATGGTGTAG | | | |
| | AAAGTATATGAAATCCGGTAACGTGAAA | | | |
| | GACCTGCTCCAGGCCTGGGACCTTTATT | | | |
| | ACCATGTGTTCAGGCGGATCAGTAAG | | | |
| Linker | gtcgag | 2276 | VE | 2277 |
| FKBP_{WT}_{^{′}} | GGCGTCCAAGTCGAAACCATTAGTCCC | 2278 | GVQVETISPGDGRTFPKRGQ | 2279 |
| | GGCGATGGCAGAACATTTCCTACAAGG | | TCVVHYTGMLEDGKKFDSSR | |
| | GGACAAACATGTGTCGTCCATTATACAG | | DRNKPFKFMLGKQEVIRGWE | |
| | GCATGTTGGAGGACGGCAAAAAGTTCG | | EGVAQMSVGQRAKLTISPDYA | |
| | ACAGTAGTAGAGATCGCAATAAACCTTT | | YGATGHPGIIPPHATLVFDVEL | |
| | CAAATTCATGTTGGGAAAACAAGAAGTC | | LKLE | |
| | ATTAGGGGATGGGAGGAGGGCGTGGCT | | | |
| | CAAATGTCCGTCGGCCAACGCGCTAAG | | | |
| | CTCACCATCAGCCCCGACTACGCATAC | | | |
| | GGCGCTACCGGACATCCCGGAATTATT | | | |
| | CCCCCTCACGCTACCTTGGTGTTTGACG | | | |
| | TCGAACTGTTGAAGCTCGAA | | | |
| MyD88 | atggctgcaggaggtcccggcgcggggtctgcggccccggt | 2280 | MAAGGPGAGSAAPVSSTSSL | 2281 |
| | ctcctccacatcctcccttcccctggctgctctcaacatg | | PLAALNMRVRRRLSLFLNVRT | |
| | cgagtgcggcgccgcctgtctctgttcttgaacgtgcggac | | QVAADWTALAEEMDFEYLEIR | |
| | acaggtggcggccgactggaccgcgctggcggaggag | | QLETQADPTGRLLDAWQGRP | |
| | atggactttgagtacttggagatccggcaactggagacac | | GASVGRLLDLLTKLGRDDVLL | |
| | aagcggaccccactggcaggctgctggacgcctggcag | | ELGPSIEEDCQKYILKQQQEE | |
| | ggacgccctggcgcctctgtaggccgactgctcgatctgct | | AEKPLQVAAVDSSVPRTAELA | |
| | taccaagctgggccgcgacgacgtgctgctggagctggg | | GITTLDDPLGHMPERFDAFICY | |
| | acccagcattgaggaggattgccaaaagtatatcttgaag | | CPSDI | |
| | cagcagcaggaggaggctgagaagcctttacaggtggc | | | |
| | cgctgtagacagcagtgtcccacggacagcagagctgg | | | |
| | cgggcatcaccacacttgatgaccccctggggcatatgcc | | | |
| | tgagcgtttcgatgccttcatctgctattgccccagcgacatc | | | |
| Linker | gtcgag | 2282 | VE | 2283 |
| CD40 | aaaaaggtggccaagaagccaaccaataaggcccccc | 2284 | KKVAKKPTNKAPHPKQEPQEI | 2285 |
| | accccaagcaggagccccaggagatcaattttcccgacg | | NFPDDLPGSNTAAPVQETLH | |
| | atcttcctggctccaacactgctgctccagtgcaggagactt | | GCQPVTQEDGKESRISVQER | |
| | tacatggatgccaaccggtcacccaggaggatggcaaa | | Q | |
| | gagagtcgcatctcagtgcaggagagacag | | | |
| Linker | ggatctgga | 2286 | GSG | 2287 |
| P2A | GCAACGAATTTTTCCCTGCTGAAACAGG | 2288 | ATNFSLLKQAGDVEENPGP | 2289 |
| | CAGGGGACGTAGAGGAAAATCCTGGTC | | | |
| | CT | | | |
| Linker | atgcatATGCTGGAG | 2290 | MHMLE | 2291 |
| FKBPv | GGAGTGCAGGTGGAGACTATTAGCCCC | 2292 | GVQVETISPGDGRTFPKRGQ | 2293 |
| | GGAGATGGCAGAACATTCCCCAAAAGA | | TCVVHYTGMLEDGKKVDSSR | |
| | GGACAGACTTGCGTCGTGCATTATACTG | | DRNKPFKFMLGKQEVIRGWE | |
| | GAATGCTGGAAGACGGCAAGAAGGTGG | | EGVAQMSVGQRAKLTISPDYA | |
| | ACAGCAGCCGGGACCGAAACAAGCCCT | | YGATGHPGIIPPHATLVFDVEL | |
| | TCAAGTTCATGCTGGGGAAGCAGGAAG | | LKLE | |
| | TGATCCGGGGCTGGGAGGAAGGAGTCG | | | |
| | CACAGATGTCAGTGGGACAGAGGGCCA | | | |
| | AACTGACTATTAGCCCAGACTACGCTTA | | | |
| | TGGAGCAACCGGCCACCCCGGGATCAT | | | |
| | TCCCCCTCATGCTACACTGGTCTTCGAT | | | |
| | GTGGAGCTGCTGAAGCTGGAA | | | |
| Linker | TCAGGCGGTGGCTCAGGTGTGGAC | 2294 | SGGGSGVD | 2295 |
| Δcaspase9 | GGATTTGGTGATGTCGGTGCTCTTGAGA | 2296 | GFGDVGALESLRGNADLAYIL | 2297 |
| | GTTTGAGGGGAAATGCAGATTTGGCTTA | | SMEPCGHCLIINNVNFCRESG | |
| | CATCCTGAGCATGGAGCCCTGTGGCCA | | LRTRTGSNIDCEKLRRRFSSL | |
| | CTGCCTCATTATCAACAATGTGAACTTCT | | HFMVEVKGDLTAKKMVLALLE | |
| | GCCGTGAGTCCGGGCTCCGCACCCGCA | | LARQDHGALDCCVVVILSHGC | |
| | CTGGCTCCAACATCGACTGTGAGAAGTT | | QASHLQFPGAVYGTDGCPVS | |
| | GCGGCGTCGCTTCTCCTCGCTGCATTTC | | VEKIVNIFNGTSCPSLGGKPKL | |
| | ATGGTGGAGGTGAAGGGCGACCTGACT | | FFIQACGGEQKDHGFEVASTS | |
| | GCCAAGAAAATGGTGCTGGCTTTGCTG | | PEDESPGSNPEPDATPFQEG | |
| | GAGCTGGCGCgGCAGGACCACGGTGCT | | LRTFDQLDAISSLPTPSDIFVS | |
| | CTGGACTGCTGCGTGGTGGTCATTCTCT | | YSTFPGFVSVVRDPKSGSVVYV | |
| | CTCACGGCTGTCAGGCCAGCCACCTGC | | ETLDDIFEQWAHSEDLQSLLL | |
| | AGTTCCCAGGGGCTGTCTACGGCACAG | | RVANAVSVKGIYKQMPGCFN | |
| | ATGGATGCCCTGTGTCGGTCGAGAAGA | | FLRKKLFFKTSASRA | |
| | TTGTGAACATCTTCAATGGGACCAGCTG | | | |
| | CCCCAGCCTGGGAGGGAAGCCCAAGCT | | | |
| | CTTTTTCATCCAGGCCTGTGGTGGGGA | | | |
| | GCAGAAAGAtCATGGGTTTGAGGTGGCC | | | |
| | TCCACTTCCCCTGAAGACGAGTCCCCTG | | | |
| | GCAGTAACCCCGAGCCAGATGCCACCC | | | |
| | CGTTCCAGGAAGGTTTGAGGACCTTCG | | | |
| | ACCAGCTGGACGCCATATCTAGTTTGCC | | | |
| | CACACCCAGTGACATCTTTGTGTCCTAC | | | |
| | TCTACTTTCCCAGGTTTTGTTTCCTGGA | | | |
| | GGGACCCCAAGAGTGGCTCCTGGTACG | | | |
| | TTGAGACCCTGGACGACATCTTTGAGCA | | | |
| | GTGGGCTCACTCTGAAGACCTGCAGTC | | | |
| | CCTCCTGCTTAGGGTCGCTAATGCTGTT | | | |
| | TCGGTGAAAGGGATTTATAAACAGATGC | | | |
| | CTGGTTGCTTTAATTTCCTCCGGAAAAA | | | |
| | ACTTTTCTTTAAAACATCAGCTAGCAGA | | | |
| | GCC | | | |
| Linker | ggatctggaccgcGG | 2298 | GSGPR | 2299 |
| T2A | GAAGGCCGAGGGAGCCTGCTGACATGT | 2300 | EGRGSLLTCGDVEENPGP | 2301 |
| | GGCGATGTGGAGGAAAACCCAGGACCA | | | |
| Linker | GCATGCGCCACC | 2302 | ACAT | 2303 |
| Signal | ATGGAGTTTGGGTTGTCATGGTTGTTTC | 2304 | MEFGLSWLFLVAILKGVQCSR | 2305 |
| Peptide | TCGTCGCTATTCTCAAAGGTGTACAATG | | | |
| | CTCCCGC | | | |
| HER2(FRP | GAAGTCCAATTGCAACAGTCAGGCCCC | 2306 | EVQLQQSGPELKKPGETVKIS | 2307 |
| 5)VH | GAATTGAAAAAGCCCGGCGAAACAGTG | | CKASGYPFTNYGMNVVVKQAP | |
| | AAGATATCTTGTAAAGCCTCCGGTTACC | | GQGLKWMGWINTSTGESTFA | |
| | CTTTTACGAACTATGGAATGAACTGGGT | | DDFKGRFDFSLETSANTAYLQ | |
| | CAAACAAGCCCCTGGACAGGGATTGAA | | INNLKSEDMATYFCARVVEVYH | |
| | GTGGATGGGATGGATCAATACATCAACA | | GYVPYWGQGTTVTVSS | |
| | GGCGAGTCTACCTTCGCAGATGATTTCA | | | |
| | AAGGTCGCTTTGACTTCTCACTGGAGAC | | | |
| | CAGTGCAAATACCGCCTACCTTCAGATT | | | |
| | AACAATCTTAAAAGCGAGGATATGGCAA | | | |
| | CCTACTTTTGCGCAAGATGGGAAGTTTA | | | |
| | TCACGGGTACGTGCCATACTGGGGACA | | | |
| | AGGAACGACAGTGACAGTTAGTAGC | | | |
| Flex | GGCGGTGGAGGCTCCGGTGGAGGCGG | 2308 | GGGGSGGGGSGGGGS | 2309 |
| | CTCTGGAGGAGGAGGTTCA | | | |
| HER2(FRP | GACATCCAATTGACACAATCACACAAAT | 2310 | EVQLVEYGGGLVQPGGSLRL | 2311 |
| 5)VL | TTCTCTCAACTTCTGTAGGAGACAGAGT | | SCAASGFNIKDYYIHVVVRQAP | |
| | GAGCATAACCTGCAAAGCATCCCAGGA | | GKGLEWVAWIDPENGDTEFV | |
| | CGTGTACAATGCTGTGGCTTGGTACCAA | | PKFQGRATMSADTSKNTAYL | |
| | CAGAAGCCTGGACAATCCCCAAAATTGC | | QMNSLRAEDTAVYYCKTGGF | |
| | TGATTTATTCTGCCTCTAGTAGGTACACT | | WGQGTLVTVSS | |
| | GGGGTACCTTCTCGGTTTACGGGCTCT | | | |
| | GGGTCCGGACCAGATTTCACGTTCACAA | | | |
| | TCAGTTCCGTTCAAGCTGAAGACCTCGC | | | |
| | TGTTTATTTTTGCCAGCAGCACTTCCGA | | | |
| | ACCCCTTTTACTTTTGGCTCAGGCACTA | | | |
| | AGTTGGAAATCAAGGCTTTG | | | |
| Linker | atgcat | 2312 | MH | 2313 |
| CD34 | GAACTTCCTACTCAGGGGACTTTCTCAA | 2314 | ELPTQGTFSNVSTNVS | 2315 |
| epitope | ACGTTAGCACAAACGTAAGT | | | |
| CD3ζ | AGAGTGAAGTTCAGCAGGAGCGCAGAC | 2316 | RVKFSRSADAPAYQQGQNQL | 2317 |
| | GCCCCCGCGTACCAGCAGGGCCAGAAC | | YNELNLGRREEYDVLDKRRG | |
| | CAGCTCTATAACGAGCTCAATCTAGGAC | | RDPEMGGKPRRKNPQEGLYN | |
| | GAAGAGAGGAGTACGATGTTTTGGACAA | | ELQKDKMAEAYSEIGMKGER | |
| | GAGACGTGGCCGGGACCCTGAGATGG | | RRGKGHDGLYQGLSTATKDT | |
| | GGGGAAAGCCGAGAAGGAAGAACCCTC | | YDALHMQALPPR | |
| | AGGAAGGCCTGTACAATGAACTGCAGA | | | |
| | AAGATAAGATGGCGGAGGCCTACAGTG | | | |
| | AGATTGGGATGAAAGGCGAGCGCCGGA | | | |
| | GGGGCAAGGGGCACGATGGCCTTTACC | | | |
| | AGGGTCTCAGTACAGCCACCAAGGACA | | | |
| | CCTACGACGCCCTTCACATGCAAGCTCT | | | |
| | TCCACCTCGT | | | |

APPENDIX 35
pBP1796—pSFG—FKBP_{wt}.FRB_{L}-MC. P2A.FKBPv.ΔC9.T2A-αPSCA.Q.CD8stm.ζ

| | | Seq | | Seq |
|---|---|---|---|---|
| | | ID | | ID |
| Fragment | Nucleotide | No: | Peptide | No: |
| FKBP_{WT}_{^{′}} | atgGGCGTCCAAGTCGAAACCATTAGTC | 2318 | MGVQVETISPGDGRTFPKRG | 2319 |
| | CCGGCGATGGCAGAACATTTCCTACAA | | QTCVVHYTGMLEDGKKFDSS | |
| | GGGGACAAACATGTGTCGTCCATTATAC | | RDRNKPFKFMLGKQEVIRGW | |
| | AGGCATGTTGGAGGACGGCAAAAAGTT | | EEGVAQMSVGQRAKLTISPDY | |
| | CGACAGTAGTAGAGATCGCAATAAACCT | | AYGATGHPGIIPPHATLVFDVE | |
| | TTCAAATTCATGTTGGGAAAACAAGAAG | | LLKLE | |
| | TCATTAGGGGATGGGAGGAGGGCGTGG | | | |
| | CTCAAATGTCCGTCGGCCAACGCGCTA | | | |
| | AGCTCACCATCAGCCCCGACTACGCAT | | | |
| | ACGGCGCTACCGGACATCCCGGAATTA | | | |
| | TTCCCCCTCACGCTACCTTGGTGTTTGA | | | |
| | CGTCGAACTGTTGAAGCTCGAA | | | |
| Linker | GGATCAGGCGGTGGCAGCGGCCAATTG | 2320 | gSGGGSGel | 2321 |
| FRB_{L} | ATGTTGGAAATGTGGCATGAAGGGTTG | 2322 | MLEMVVHEGLEEASRLYFGER | 2323 |
| | GAAGAAGCTTCAAGGCTGTACTTCGGA | | NVKGMFEVLEPLHAMMERGP | |
| | GAGAGGAACGTGAAGGGCATGTTTGAG | | QTLKETSFNQAYGRDLMEAQ | |
| | GTTCTTGAACCTCTGCACGCCATGATGG | | EWCRKYMKSGNVKDLLQAW | |
| | AACGGGGACCGCAGACACTGAAAGAAA | | DLYYHVFRRISK | |
| | CCTCTTTTAATCAGGCCTACGGCAGAGA | | | |
| | CCTGATGGAGGCCCAAGAATGGTGTAG | | | |
| | AAAGTATATGAAATCCGGTAACGTGAAA | | | |
| | GACCTGCTCCAGGCCTGGGACCTTTATT | | | |
| | ACCATGTGTTCAGGCGGATCAGTAAG | | | |
| Linker | ggcagtggaGGCGGG | 2324 | Gsgggm | 2325 |
| MyD88 | atggctgcaggaggtcccggcgcggggtctgcggcccc | 2326 | MAAGGPGAGSAAPVSSTSSL | 2327 |
| | ggtctcctccacatcctcccttcccctggctgctctcaacatg | | PLAALNMRVRRRLSLFLNVRT | |
| | cgagtgcggcgccgcctgtctctgttcttgaacgtgcggac | | QVAADWTALAEEMDFEYLEIR | |
| | acaggtggcggccgactggaccgcgctggcggaggag | | QLETQADPTGRLLDAWQGRP | |
| | atggactttgagtacttggagatccggcaactggagacac | | GASVGRLLDLLTKLGRDDVLL | |
| | aagcggaccccactggcaggctgctggacgcctggcag | | ELGPSIEEDCQKYILKQQQEE | |
| | ggacgccctggcgcctctgtaggccgactgctcgatctgct | | AEKPLQVAAVDSSVPRTAELA | |
| | taccaagctgggccgcgacgacgtgctgctggagctggg | | GITTLDDPLGHMPERFDAFICY | |
| | acccagcattgaggaggattgccaaaagtatatcttgaag | | CPSDI | |
| | cagcagcaggaggaggctgagaagcctttacaggtggc | | | |
| | cgctgtagacagcagtgtcccacggacagcagagctgg | | | |
| | cgggcatcaccacacttgatgaccccctggggcatatgcc | | | |
| | tgagcgtttcgatgccttcatctgctattgccccagcgacatc | | | |
| Linker | gtcgag | 2328 | VE | 2329 |
| CD40 | aaaaaggtggccaagaagccaaccaataaggcccccc | 2330 | KKVAKKPTNKAPHPKQEPQEI | 2331 |
| | accccaagcaggagccccaggagatcaattttcccgacg | | NFPDDLPGSNTAAPVQETLH | |
| | atcttcctggctccaacactgctgctccagtgcaggagactt | | GCQPVTQEDGKESRISVQER | |
| | tacatggatgccaaccggtcacccaggaggatggcaaa | | Q | |
| | gagagtcgcatctcagtgcaggagagacag | | | |
| Linker | ggatctgga | 2332 | GSG | 2333 |
| P2A | GCAACGAATTTTTCCCTGCTGAAACAGG | 2334 | ATNFSLLKQAGDVEENPGP | 2335 |
| | CAGGGGACGTAGAGGAAAATCCTGGTC | | | |
| | CT | | | |
| Linker | atgcatATGCTGGAG | 2336 | MHMLE | 2337 |
| FKBPv | GGAGTGCAGGTGGAGACTATTAGCCCC | 2338 | GVQVETISPGDGRTFPKRGQ | 2339 |
| | GGAGATGGCAGAACATTCCCCAAAAGA | | TCVVHYTGMLEDGKKVDSSR | |
| | GGACAGACTTGCGTCGTGCATTATACTG | | DRNKPFKFMLGKQEVIRGVVE | |
| | GAATGCTGGAAGACGGCAAGAAGGTGG | | EGVAQMSVGQRAKLTISPDYA | |
| | ACAGCAGCCGGGACCGAAACAAGCCCT | | YGATGHPGIIPPHATLVFDVEL | |
| | TCAAGTTCATGCTGGGGAAGCAGGAAG | | LKLE | |
| | TGATCCGGGGCTGGGAGGAAGGAGTC | | | |
| | GCACAGATGTCAGTGGGACAGAGGGCC | | | |
| | AAACTGACTATTAGCCCAGACTACGCTT | | | |
| | ATGGAGCAACCGGCCACCCCGGGATCA | | | |
| | TTCCCCCTCATGCTACACTGGTCTTCGA | | | |
| | TGTGGAGCTGCTGAAGCTGGAA | | | |
| Linker | TCAGGCGGTGGCTCAGGTGTGGAC | 2340 | SGGGSGVD | 2341 |
| Δcaspase9 | GGATTTGGTGATGTCGGTGCTCTTGAGA | 2342 | GFGDVGALESLRGNADLAYIL | 2343 |
| | GTTTGAGGGGAAATGCAGATTTGGCTTA | | SMEPCGHCLIINNVNFCRESG | |
| | CATCCTGAGCATGGAGCCCTGTGGCCA | | LRTRTGSNIDCEKLRRRFSSL | |
| | CTGCCTCATTATCAACAATGTGAACTTCT | | HFMVEVKGDLTAKKMVLALLE | |
| | GCCGTGAGTCCGGGCTCCGCACCCGCA | | LARQDHGALDCCVVVILSHGC | |
| | CTGGCTCCAACATCGACTGTGAGAAGTT | | QASHLQFPGAVYGTDGCPVS | |
| | GCGGCGTCGCTTCTCCTCGCTGCATTTC | | VEKIVNIFNGTSCPSLGGKPKL | |
| | ATGGTGGAGGTGAAGGGCGACCTGACT | | FFIQACGGEQKDHGFEVASTS | |
| | GCCAAGAAAATGGTGCTGGCTTTGCTG | | PEDESPGSNPEPDATPFQEG | |
| | GAGCTGGCGCgGCAGGACCACGGTGCT | | LRTFDQLDAISSLPTPSDIFVS | |
| | CTGGACTGCTGCGTGGTGGTCATTCTCT | | YSTFPGFVSVVRDPKSGSVVYV | |
| | CTCACGGCTGTCAGGCCAGCCACCTGC | | ETLDDIFEQWAHSEDLQSLLL | |
| | AGTTCCCAGGGGCTGTCTACGGCACAG | | RVANAVSVKGIYKQMPGCFN | |
| | ATGGATGCCCTGTGTCGGTCGAGAAGA | | FLRKKLFFKTSASRA | |
| | TTGTGAACATCTTCAATGGGACCAGCTG | | | |
| | CCCCAGCCTGGGAGGGAAGCCCAAGCT | | | |
| | CTTTTTCATCCAGGCCTGTGGTGGGGA | | | |
| | GCAGAAAGAtCATGGGTTTGAGGTGGCC | | | |
| | TCCACTTCCCCTGAAGACGAGTCCCCTG | | | |
| | GCAGTAACCCCGAGCCAGATGCCACCC | | | |
| | CGTTCCAGGAAGGTTTGAGGACCTTCG | | | |
| | ACCAGCTGGACGCCATATCTAGTTTGCC | | | |
| | CACACCCAGTGACATCTTTGTGTCCTAC | | | |
| | TCTACTTTCCCAGGTTTTGTTTCCTGGA | | | |
| | GGGACCCCAAGAGTGGCTCCTGGTACG | | | |
| | TTGAGACCCTGGACGACATCTTTGAGCA | | | |
| | GTGGGCTCACTCTGAAGACCTGCAGTC | | | |
| | CCTCCTGCTTAGGGTCGCTAATGCTGTT | | | |
| | TCGGTGAAAGGGATTTATAAACAGATGC | | | |
| | CTGGTTGCTTTAATTTCCTCCGGAAAAA | | | |
| | ACTTTTCTTTAAAACATCAGCTAGCAGA | | | |
| | GCC | | | |
| Linker | ggatctggaccgcGG | 2344 | GSGPR | 2345 |
| T2A | GAAGGCCGAGGGAGCCTGCTGACATGT | 2346 | EGRGSLLTCGDVEENPGP | 2347 |
| | GGCGATGTGGAGGAAAACCCAGGACCA | | | |
| Linker | CCACGG | 2348 | PR | 2349 |
| Signal | ATGGAGTTTGGACTTTCTTGGTTGTTTTT | 2350 | MEFGLSWLFLVAILKGVQCSR | 2351 |
| Peptide | GGTGGCAATTCTGAAGGGTGTCCAGTG | | | |
| | TAGCAGG | | | |
| PSCA(A11) | GACATCCAACTGACGCAAAGCCCATCTA | 2352 | DIQLTQSPSTLSASMGDRVTIT | 2353 |
| VL | CACTCAGCGCTAGCATGGGGGACAGGG | | CSASSSVRFIHVVYQQKPGKA | |
| | TCACAATCACGTGCTCTGCCTCAAGTTC | | PKRLIYDTSKLASGVPSRFSG | |
| | CGTTAGGTTTATCCATTGGTATCAGCAG | | SGSGTDFTLTISSLQPEDFATY | |
| | AAACCTGGAAAGGCCCCAAAAAGACTG | | YCQQWGSSPFTFGQGTKVEI | |
| | ATCTATGATACCAGCAAGCTGGCTTCCG | | K | |
| | GAGTGCCCTCAAGGTTCTCAGGATCTG | | | |
| | GCAGTGGGACCGATTTCACCCTGACAA | | | |
| | TTAGCAGCCTTCAGCCAGAGGATTTCGC | | | |
| | AACCTATTACTGTCAGCAATGGGGGTCC | | | |
| | AGCCCATTCACTTTCGGCCAAGGAACAA | | | |
| | AGGTGGAGATAAAA | | | |
| Flex | GGCGGAGGAAGCGGAGGTGGGGGC | 2354 | gggsgggg | 2355 |
| PSCA(A11) | GAGGTGCAGCTCGTGGAGTATGGCGGG | 2356 | EVQLVEYGGGLVQPGGSLRL | 2357 |
| VH | GGCCTGGTGCAGCCTGGGGGTAGTCTG | | SCAASGFNIKDYYIHVVVRQAP | |
| | AGGCTCTCCTGCGCTGCCTCTGGCTTTA | | GKGLEVVVAWIDPENGDTEFV | |
| | ACATTAAAGACTACTACATACATTGGGT | | PKFQGRATMSADTSKNTAYL | |
| | GCGGCAGGCCCCAGGCAAAGGGCTCG | | QMNSLRAEDTAVYYCKTGGF | |
| | AATGGGTGGCCTGGATTGACCCTGAGA | | WGQGTLVTVSS | |
| | ATGGTGACACTGAGTTTGTCCCCAAGTT | | | |
| | TCAGGGCAGAGCCACCATGAGCGCTGA | | | |
| | CACAAGCAAAAACACTGCTTATCTCCAA | | | |
| | ATGAATAGCCTGCGAGCTGAAGATACA | | | |
| | GCAGTCTATTACTGCAAGACGGGAGGA | | | |
| | TTCTGGGGCCAGGGAACTCTGGTGACA | | | |
| | GTTAGTTCC | | | |
| Linker | GGATCC | 2358 | gs | 2359 |
| CD34 | GAACTTCCTACTCAGGGGACTTTCTCAA | 2360 | ELPTQGTFSNVSTNVS | 2361 |
| epitope | ACGTTAGCACAAACGTAAGT | | | |
| CD3ζ | AGAGTGAAGTTCAGCAGGAGCGCAGAC | 2362 | RVKFSRSADAPAYQQGQNQL | 2363 |
| | GCCCCCGCGTACCAGCAGGGCCAGAAC | | YNELNLGRREEYDVLDKRRG | |
| | CAGCTCTATAACGAGCTCAATCTAGGAC | | RDPEMGGKPRRKNPQEGLYN | |
| | GAAGAGAGGAGTACGATGTTTTGGACA | | ELQKDKMAEAYSEIGMKGER | |
| | AGAGACGTGGCCGGGACCCTGAGATGG | | RRGKGHDGLYQGLSTATKDT | |
| | GGGGAAAGCCGAGAAGGAAGAACCCTC | | YDALHMQALPPR | |
| | AGGAAGGCCTGTACAATGAACTGCAGA | | | |
| | AAGATAAGATGGCGGAGGCCTACAGTG | | | |
| | AGATTGGGATGAAAGGCGAGCGCCGGA | | | |
| | GGGGCAAGGGGCACGATGGCCTTTACC | | | |
| | AGGGTCTCAGTACAGCCACCAAGGACA | | | |
| | CCTACGACGCCCTTCACATGCAAGCTCT | | | |
| | TCCACCTCGT | | | |

### Example 30: Dual Control of Apoptosis

The present Example provides examples of chimeric pro-apoptotic polypeptides that include dual molecular switches, providing a choice of ligand for activating apoptosis. Chimeric dual-controlled Caspase-9 polypeptides were prepared and assayed for apoptotic activity.

In this example, in vitro data is provided that compares the apoptotic induction of various Caspase-9 molecular switches in response to rimiducid and rapamycin treatment in 293 and primary human T cells. T cells expressing these three caspase-9 switches when introduced into NSG mice are efficiently eliminated within 24 hours of exposure to their respective activating ligands. Finally, dose titration of the FRB.FKBP_{V}.ΔC9 switch in vivo demonstrated that both rimiducid and rapamycin stimulated efficient removal of T cells with drug concentrations as low as 1 mg/kg.

### Methods

Peripheral blood mononuclear cells (PBMCs) were isolated from buffy coats obtained through the Gulf Coast Regional Blood Center. Buffy coats tested negative for infectious viral pathogens.

### Activation and Transduction of T Cells

Production of retrovirus by transient transfection of 293T, and activation of T cells were performed essentially as discussed herein. T cells were transduced with pBP1501, pBP0220, pBP1310, pBP1311, pBP1327, pBP1328 vectors.

### Phenotyping and In Vivo Cell Enumeration

Transduction efficiency was determined by flow cytometry using anti-CD3-PerCP.Cy5.5 and anti-CD19-APC antibodies. Following mouse sacrifice, total transduced T cell numbers in the spleens were calculated by counting total splenocyte numbers and multiplying by the percentage of CD3⁺CD34⁺ T cells observed by flow cytometry. To examine the phenotype of T cells in mice, spleens were isolated and single-cell suspensions were made by lysing red cells with ammonia chloride/potassium (ACK)-based lysis buffer followed by mechanical dissociation through a 70-μm nylon filter. Cells were subsequently stained with the following antibodies: anti-hCD3-PerCP.Cy5.5, anti-hCD19-APC, and anti-mCD45RA-BV510.

### SRα SEAP Assay in 293 Cells

On day 0, 5×10⁵ 293 cells were seeded onto 6-well plates in 2 ml DMEM medium (10% FBS+1% pen/strep). On day 1, cells were co-transfected with 1 μg each of pBP1501, pBP0220, pBP1310, pBP1311, pBP1327, pBP1328 vectors and the SRα-SEAP reporter plasmid (pBP0046). On day 2, cells were collected, and seeded onto 96-well plates containing 2× concentrated half-log drug dilutions and also analyzed by FACS for transfection efficiency. On day 3, the drug-treated cells were heat inactivated at 68° C. for 1 hour and supernatants were added to black 96-well plate containing 1 mM MUP substrate (2× concentration) diluted in 2M diethanolamine. The plates were incubated at 37° C. for 30 min and absorbance at 405 nm was measured.

### Western Blot Analysis

After transduction with the appropriate retrovirus, 6×10⁶ T cells were seeded per well into 6-well plates in 3 ml CTL medium. Twenty-four hours later, cells were collected, washed in cold PBS, and lysed in RIPA Lysis and Extraction Buffer (Thermo, 89901), containing 1× Halt Protease Inhibitor Cocktail (Thermo, 87786) on ice for 30 min in the plated. The lysates were centrifuged at 16,000×g for 20 min at 4° C. and the supernatants were transferred to new Eppendorf tubes. Protein assays were performed using the Pierce BCA Protein Assay Kit (Thermo, 23227) per manufacturer's recommendation. To prepare samples for SDS-PAGE, 50 μg of lysates was mixed with 4× Laemmli Sample Buffer (Bio Rad, 1610747) and heated at 95° C. for 10 min. Meanwhile, 10% SDS gels were prepared using a Bio Rad casting apparatus and 30% Acrylamide/bis Solution (Bio Rad, 160158). The samples were loaded at equal levels of total protein along with Precision Plus Protein Dual Color Standards (Bio Rad, 1610374) and run in 1× Tris/glycine Running Buffer (Bio Rad, 1610771) at 140 V for 90 min. After protein separation, gels were transferred onto PVDF membranes using Program 0 (7 min total) in the iBlot 2 device (Thermo, IB21001). Membranes were subsequently probed with primary and secondary antibodies using the iBind Flex Western Device (Thermo, SLF2000) according to manufacturer's recommendation. Anti-caspase-9 antibody (Thermo, PA1-12506) was used at 1:200 dilution and the secondary HRP-conjugated goat anti-rabbit IgG antibody (Thermo, A16104) was used at 1:500 dilution. The β-actin antibody (Thermo, PA1-16889) was used at 1:1000 dilution and the secondary HRP-conjugated goat anti-rabbit IgG antibody (Thermo, A16104) was used at 1:1000 dilution. The membranes were developed using the SuperSignal West Femto Maximum Sensitivity Substrate Kit (Thermo, 34096) and imaged using the GelLogic 6000 Pro camera and the CareStream MI software (v.5.3.1.16369).

### In Vitro T Cell Caspase Activation Assay Using the IncuCyte

After transduction with the appropriate retrovirus, 5×10⁴ T cells were seeded per well into a 96-well plate in the presence or absence of drugs (rimiducid or rapamycin) in CTL medium in the presence of IL-2. To enable detection of apoptosis using the IncuCyte instrument, 2 μM of IncuCyte™ Kinetic Caspase-3/7 Apoptosis reagent (Essen Bioscience, 4440) was added to each well to reach a total volume of 200 μl. The plates were centrifuged for 5 min at 400×g and placed inside the IncuCyte (Dual Color Model 4459) to monitor green fluorescence every 2-3 hours for a total of 48 hours at 10× objective. Image analysis was performed using the “Tcells_caspreagent_phase_green_10×_MLD” processing definition. The “Total Green Object Integrated Intensity” metric and the “Phase Object Confluence (Percent)” were used to quantify caspase activation. Each condition was performed in duplicate and each well was imaged at 4 different locations.

$$caspase 3 / 7 activation readout = \frac{\begin{matrix}Metric : Total Green Object \\ Integrated Intensity ( GCU \times {μm}^{2} / Image )\end{matrix}}{Metric : Phase Object Confluence ( Percent )}$$

### Animal Model

8-week-old female, immune-deficient mice (NOD.CgPrkdc^{scid}Il2^{tm1Wjl}/SzJ; NSG) were injected IV with 1×10⁶ T cells in 100 p1 PBS. Mice were subjected to IVIS imaging ˜4 hrs after T cell injection (˜14 hrs post-drug administration). The following day, mice were imaged just before drug injection (0 hrs), then injected IP with vehicle, rimiducid diluted in solutol and PBS, or rapamycin diluted in “PT”. Mice were imaged again at 5-6 hrs, and 24 hrs after drug injection. Mice were sacrificed and spleens were removed for FACS analysis.

### In Vivo Bioluminescence Imaging

Mice were imaged for firefly luciferase-derived bioluminescence at the indicated time points relative to administration of drug or vehicle.

### Results

### Topology of FRB and FKBP in Chimeric Caspase-9 Polypeptides

Since the order and spacing of signaling elements and binding domains may affect outcomes, the order of ligand-binding domains with the inducible chimeric Caspase-9 polypeptides was examined (FRB.FKBP.ΔC9 (pBP1310) and FKBP.FRB.ΔC9 (pBP1311)) (FIG. 106A). A caspase activation assay that utilizes the caspase 3/7 green reagent (in which caspase activity is captured by the cleavage of the peptide reagent which releases a green fluorophore, green fluorescent emission thereby marks cells undergoing apoptosis) revealed that FRB.FKBP.ΔC9 is slightly more sensitive than FKBP.FRB.ΔC9 to rapamycin-mediated initiation of apoptosis in T cells (FIG. 106B). This modest difference may be attributed to higher FRB.FKBP.ΔC9 protein level compared to that of the FKBP.FRB.ΔC9 (FIG. 106C).

Since the chimeric iRC9 caspase polypeptide contains the wild-type FKBP domain, it was necessary to determine the concentration of rimiducid capable of triggering dimerization and caspase activation. In this assay, 293 cells were transiently transfected with vectors expressing FKBPv36 Caspase-9 (iC9) and the two similar rapamycin-inducible variants (FRB.FKBP.ΔC9 and FKBP.FRB.ΔC9) (FIG. 107) and treated with half-log dilution of either rapamycin or rimiducid. Cells underwent either a caspase activation assay in the presence of caspase 3/7 green reagent and monitored by IncuCyte or alternatively, Rapamycin-mediated cell death was measured indirectly by a secreted alkaline phosphatase (SEAP) assay using a constitutive SRα-SEAP reporter. Functionally, the rapamycin inducible and the rimiducid inducible chimeric Caspase-9 polypeptides appear to induce caspase cleavage with similar kinetics and threshold when activated by their respective suicide drugs (FIG. 107A). In contrast, data obtained from the SEAP assay demonstrates that the rimiducid-inducible switch in the iC9 chimeric caspase polypeptide is more sensitive to activation at low rimiducid concentrations compared with the rapamycin-inducible caspase-9 switches (iRC9) at low rapamycin concentrations (FIG. 107B).

The rapamycin inducible chimeric Caspase-9 polypeptide, iRC9, is highly active even in the presence of as little as 100 μM rapamycin, with some efficacy at even lower drug levels, albeit with slower kinetics. When comparing the two iRC9 polypeptides, FRB.FKBP.ΔC9 versus FKBP.FRB.ΔC9, FRB.FKBP.ΔC9 is active at lower rapamycin concentration than FKBP.FRB.ΔC9, consistent with data obtained in FIG. 106B. Finally, the iRC9 chimeric Caspase-9 polypeptide is insensitive to rimiducid below 100 nM making it feasible to combine this rapamycin-induced off-switch with another rimiducid-medicated switch (for example, iMC).

Chimeric iRmC9-Expressing T Cells can be Activated by Both Rimiducid and Rapamycin In Vitro.

The iRmC9 (FRB.F_{V}.ΔC9 (pBP1327) and F_{V}.FRB.ΔC9 (pBP1328)) were generated by mutating the FKBP domain within iRC9 to F36V to accommodate rimiducid binding. A SRα-SEAP assay was performed to assess the drug specificity of the 3 off-switches: iC9 (pBP220), iRC9s (pBP1310 & 1311), and iRmC9 (pBP1327 & pBP1328). The plasmid pBP1501 contains only the ΔC9 domain and serves as a negative control for drug induction (FIG. 106A). Rimiducid can activate both iC9 and iRmC9 switches but requires >100 nM ligand to activate the iRC9 switch (FIG. 108A). Conversely, rapamycin can activate both iRC9 and iRmC9 switches but is not able to induce dimerization of iC9 even at 1000 nM concentration.

To determine the functionality of these switches in activated T cells, retroviral supernatants were produced and transduced into PBMCs activated from 3 separate donors. T cells expressing the different caspase-9 switches were subjected to a killing assay with increasing doses of rimiducid and rapamycin in the presence of caspase 3/7 green reagent and monitored by IncuCyte (FIG. 108B). As observed by SRα-SEAP assay, rimiducid can activate iC9 and iRmC9 but not iRC9, which comprises the wild type FKBP12, while rapamycin is able to activate iRC9 and iRmC9, but not iC9. Negative control ΔC9 alone (pBP1501) was not active in the presence of either rimiducid or rapamycin. Of note, rimiducid activates FRB.F_{V}.ΔC9 (pBP1327) with greater efficiency than F_{V}.FRB.ΔC9 (pBP1328), possibly due to the Fv domain being proximal to caspase-9. The protein level of the inducible caspases was determined by Western blot. iC9 is expressed at higher levels compared to both iRC9 and iRmC9 (FIG. 108C). Based on these data, the following plasmids were selected to proceed to further in vivo testing: iC9 (pBP0220), iRC9 (pBP1310), and iRmC9 (pBP1327).

iRmC9 T Cells can be Activated by Both Rimiducid and Rapamycin In Vivo.

PBMCs from donor 676 were activated and co-transduced with one of the off-switches and GFP-Fluc retroviruses. Eleven days after transduction, cells were analyzed for transduction efficiency with GFP and anti-CD3/anti-CD19 antibodies (FIG. 109A). This analysis showed that iC9 T cells were 41% GFP⁺/CD19⁺, iRC9 T cells were 65% GFP⁺/CD19⁺ and iRmC9 T cells were 51% GFP⁺/CD19⁺. The CD19⁺ MFI for the different T cell populations were: iC9=15.07, iRC9=14.38, and iRmC9=13.39. The cells were collected, counted, washed, and resuspended at 1×10⁶ cells in 100 μl PBS for each tail vein mouse injection (Table 10) (time=−18 hr). The next day, 5 mg/kg rimiducid (dissolved in solutol and PBS) or 10 mg/kg rapamycin (dissolved in detergent-based excipient “PT”) 10% PEG-400+17% Tween-80) were injected intraperitoneally into each respective group (time=0 hr). IVIS imaging was performed at −14, 0, 5, 24 and 29 hours. Mice were sacrificed and spleens were collected for FACS analysis with hCD3, hCD19 and mCD45 antibodies. Rimiducid administration induced significant removal of IC9 and iRmC9 T cells while rapamycin induced removal of iRC9 and iRmC9 T cells (FIGS. 109B & C). The relatively high level of BLI signal detected in the iC9 group treated with rimiducid may be attributed to the high single GFP⁺ population (41%) in the transduced T cells (FIG. 109A). Interestingly, in the iC9-expressing T cell group treated with rapamycin, IVIS imaging shows higher signal compared to the respective no drug group, suggesting that the rapamycin vehicle that is composed of the PT might boost the bioluminescence detected. Analysis of splenocytes revealed that ˜20% of iC9 T cells remained after rimiducid treatment compared to those in treated with no drug- or rapamycin-treated groups (FIG. 109D). Similarly, at 24 hours, approximately ˜25% of iRC9 T cells remained following rapamycin treatment compared to those in the no drug- and rimiducid-treated groups. In the iRmC9 group, ˜50% and ˜40% of the iRmC9 T cells remained following rimiducid or rapamycin administration, respectively. The higher percentage of remaining iRmC9 T cells observed may be due to an artifact of normalizing the no drug group. In the graph that plots the CD19⁺ MFI of splenocytes (FIG. 109D, right graph), iRmC9 T cells had lower CD19⁺ MFI as seen before injection compared to the other groups, and the T cells that remained in the spleens post-drug treatment had similar CD19⁺ MFIs to the iC9 and iRC9-treated groups.

Drug Titration of Rimiducid and Rapamycin in Mice Bearing iRmC9 T Cells.

The iRmC9 construct represents an ideal switch that can allow for direct comparison of rimiducid versus rapamycin-induced killing kinetics in the same molecule. In this experiment, iRmC9 T cells were produced by co-transduction with pBP1327 and GFP-Fluc retroviruses from donor 584. Ten days post-transduction, FACS analysis indicated that 73% of the cells were GFP⁺/CD19⁺ and the CD19⁺ MFI was 15.23 (FIG. 110A). Ten million iRmC9 T cells were injected IV per mouse (Table 10) (time=−14 hr). The next day, rimiducid (dissolved in solutol and PBS) or rapamycin (dissolved in PT) were injected intraperitoneally into each respective group (time=0 hr). Vehicle groups received either PBS, 25% solutol in PBS or 5% DMA in PT. IVIS imaging was performed at −10, 0, 6, and 24 hours. Mice were sacrificed and spleens were collected for FACS analysis with hCD3, hCD19 and mCD45 antibodies. IVIS imaging for the rimiducid dose titration shows dose-dependent removal of iRmC9 T cells (FIGS. 110B & C). In contrast, IVIS imaging in the rapamycin-dosed groups shows an unexpected increase in IVIS signal detected that is most pronounced in the vehicle-treated group, but is not observed in the PBS-treated group (FIG. 110B). This observation is similar to that observed in the previous experiment (FIG. 109B) and could be due to the components of the PT. Splenocyte analysis however showed a similar dose-response with regards to deletion of iRmC9-modified T cells by rimiducid or rapamycin (FIG. 110D).

FIG. 106. Topology of FRB and FKBP in iRC9. (FIG. 106A) PBMCs from donor 920 were activated and transduced with pBP1310 and pBP1311 vectors. (FIG. 106B) Five days post-transduction, T cells were seeded on 96-well plates with 0, 0.8, 4 and 20 nM rapamycin. Additionally, 2 μM caspase 3/7 green reagent was added to monitor caspase cleavage by the IncuCyte. Line graphs depict caspase activation over 24 hours post-rapamycin treatment of FRB.FKBP.ΔC9 versus FKBP.FRB.ΔC9. (FIG. 106C) Protein expression of the iRC9 T cells was analyzed by Western blot using antibodies to hCaspase-9 and β-actin.

FIG. 107. High (>100 nM) rimiducid concentration is required to activate iRC9. 293 cells were seeded at 300,000 cells/well in a 6-well plate and allowed to grow for 2 days. After 48 h, cells were transfected with 1 μg of experimental plasmids. Cells were harvested 48 h after transfection and diluted 2.5× their original volume. (FIG. 107A) For the Incucyte/casp3/7 assay, 50 μl of cells were plated per well including either rimiducid or rapamycin drug and caspase 3/7 green reagent (2.5 μM final concentration). (FIG. 107B) For the SEAP assays, 100 μl of cells were plated in a 96-well plate with (half-log) rimiducid (or rapamycin) drug dilutions and ˜18 h after drug exposure, plates were heat-inactivated before substrate (4-MUP) addition.

FIG. 108. iRmC9 T cells can be activated by both rimiducid and rapamycin in vitro. (FIG. 108A) The SRα SEAP assay was performed by co-transfecting 293 cells with the pBP1501, 220, 1310, 1311, 1327, 1328 vectors and the SRα-SEAP reporter plasmid. (FIG. 108B) For the Incucyte/casp3/7 assay, T cells were seeded on 96-well plates with increasing rimiducid and rapamycin concentrations in the presence of 2 μM caspase 3/7 green reagent to monitor caspase cleavage by the IncuCyte. (FIG. 108C) Protein expression of the iRC9 T cells was analyzed by Western blot using antibodies to hCaspase-9 and β-actin.

FIG. 109. iRmC9 T cells can be activated by both rimiducid and rapamycin in vivo. PBMCs from donor 676 were activated and co-transduced with retroviruses encoding the pBP0220, 1310, 1327 vectors and the GFP-Fluc plasmid. (FIG. 109A) Eleven days post transduction, the cells were analyzed for CD19 and GFP transduction efficiency prior to injection into mice. (FIGS. 109B & C) NSG mice were injected i.v. with 107 T cells co-transduced with GFP-Fluc per mouse and suicide drugs were injected i.p. the next day. Bioluminescence of cells was assessed at −14, 0, 5, 24, and 29 hours post-drug administration. (FIG. 109D) At 29-h post-drug treatment, mice were euthanized and spleens were collected for flow cytometry analysis with antibodies to hCD3, hCD34, and mCD45

FIG. 110. Drug titration of rimiducid and rapamycin in mice bearing iRmC9 T cells. PBMCs from donor 584 were activated and co-transduced with retroviruses encoding the pBP1327 vector and the GFP-Fluc plasmid. (FIG. 110A) Ten days post-transduction, the cells were analyzed for CD19 and GFP transduction efficiency prior to injection into mice. (FIGS. 110B & C) NSG mice were injected i.v. with 1×107 T cells co-transduced with GFP-Fluc per mouse and suicide drugs were injected i.p. the next day. Bioluminescence of cells was assessed at −10, 0, 6, and 24 hours post drug administration. (FIG. 110D) At 24 h post-drug treatment, mice were euthanized and spleens were collected for flow cytometry analysis with antibodies to hCD3, hCD34, and mCD45.

TABLE 9
Comparing the apoptotic activation
of iC9, iRC9, and iRmC9 in vivo.

| | | | # of |
|---|---|---|---|
| Group # | T cells (GFP-Fluc) | Suicide drug | mice |
| 1 | 220 | No treatment | 3 |
| 2 | 220 | 5 mg/kg rimiducid | 5 |
| 3 | 220 | 10 mg/kg rapamycin | 3 |
| 4 | 1310 | No treatment | 3 |
| 5 | 1310 | 5 mg/kg rimiducid | 3 |
| 6 | 1310 | 10 mg/kg rapamycin | 5 |
| 7 | 1327 | No treatment | 3 |
| 8 | 1327 | 5 mg/kg rimiducid | 5 |
| 9 | 1327 | 10 mg/kg rapamycin | 5 |
| Total # of mice | | | 35 |

TABLE 10
Drug titration of rimiducid and rapamycin in mice bearing iRmC9

| | | Rimiducid | Rapamycin | # of |
|---|---|---|---|---|
| Group # | T cells (GFP-Fluc) | (mg/kg) | (mg/kg) | mice |
| 1 | 1327 (+Saline) | 0 | 0 | 3 |
| 2 | 1327 | 25% Solutol | 0 | 3 |
| | | in Saline | | |
| 3 | 1327 | 0 | 5% DMA in PT | 3 |
| 4 | 1327 | 5* | 0 | 3 |
| 5 | 1327 | 0.5* | 0 | 4 |
| 6 | 1327 | 0.05* | 0 | 4 |
| 7 | 1327 | 0.005 | 0 | 4 |
| 8 | 1327 | 0.0005 | 0 | 4 |
| 9 | 1327 | 0.00005 | 0 | 4 |
| 10 | 1327 | 0 | 10+ | 3 |
| 11 | 1327 | 0 | 1+ | 4 |
| 12 | 1327 | 0 | 0.1+ | 4 |
| 13 | 1327 | 0 | 0.01+ | 4 |
| 14 | 1327 | 0 | 0.001+ | 4 |
| 15 | 1327 | 0 | 0.0001+ | 4 |

| | |
|---|---|
| Total # of mice | 55 |

*solutol placebo added to control for 25% solutol in saline
+DMA controlled to 5% DMA in PT.

### Summary

The kinetics and efficiency of apoptosis induction following dimerizer ligand administration between three different caspase-9-enabled safety switches were compared. In general, the capacity of apoptotic induction is similar between iC9, iRC9, and iRmC9 off-switches when triggered with their respective drug(s), but there are some nuances with regards to kinetics and dose-response. Thus, these three safety-switch designs expand the toolbox of molecules that can be used for current and future clinical applications where there is a critical need for an off mechanism.

Because rapamycin and rimiducid are predicted to have different pharmacodynamic properties, one potential application for this technology could be in the choice of a ligand that can provide tissue selectivity. For example, should rimiducid be excluded from the brain due to the impermeability of the blood brain barrier, a iRmC9 switch could be activated by rapamycin. Alternatively, if titration of T cell numbers is required, the dose-response curve of one drug over another could be an important determinant of the decision of which to deploy. Moreover, if oral delivery is needed, rapamycin or analogs may be the logical choice.

### Example 31: Inducible MyD88-CD40 Costimulation Provides Ligand-Dependent Tumor Eradication by CD123-Specific Chimeric Antigen Receptor T Cells

Provided is an example of the use of one of the two molecular switches, iMC, in the context of costimulation of CD123-specific chimeric antigen receptor expressing T cells. Promising clinical results with CD19-specific chimeric antigen receptor (CAR)-directed T cells for the treatment of B cell leukemia and lymphoma suggest that CARs may be effective in other hematological malignancies, such as acute myeloid leukemia (AML).

CD123/IL-3Rα is an attractive CAR-T cell target due to its high expression on both AML blasts and leukemic stem cells (AML-LSCs). However, the antigen is also expressed at lower levels on normal stem cell progenitors presenting a major toxicity concern should CD123-specific CAR-T cells show long-term persistence.

The iMC-CAR costimulation platform iMC uses a proliferation-deficient, first generation, CD123-specific CAR together with a ligand (rimiducid (Rim))-dependent costimulatory switch (inducible MyD88/CD40 (iMC)) to provide physician-controlled eradication of CD123⁺ tumor cells and regulate long-term CAR-T cell engraftment.

Retrovirus and transduction: T cells were activated with anti-CD3/28 antibodies and subsequently transduced with a bicistronic retrovirus encoding tandem Rim-binding domains (FKBP12v36), cloned in-frame with MyD88 and CD40 cytoplasmic signaling molecules, and first generation CAR targeting CD123 (SFG-iMC-CD123.ζ) (FIG. 111).

Coculture assay: The effects of iMC costimulation on CD123-targeted CARs were assessed in coculture assays with CD123⁺, EGFPluciferase (EGFPluc)-modified leukemic cell lines (KG1, THP-1 and MOLM-13) with and without Rim using the IncuCyte live cell imaging system. IL-2 production was examined by ELISA from coculture supernatants.

Animal experiments: In vivo efficacy of iMC-CD123.ζ-modified T cells was assessed using an immune-deficient NSG tumor xenograft model. One million EGFPluc-expressing CD123⁺ THP-1 tumor cells were injected i.v. into the animals, followed by a single i.v. injection on day 7 with varying non-transduced or iMC-CD123.ζ-modified T cells. Groups receiving CAR-T cells subsequently received i.p. injections of Rim (1 mg/kg) or vehicle only on days 0 and 15 post-T cell injection. Animals were evaluated for THP-1-EGFPluc tumor burden and weight change on a weekly basis using IVIS bioluminescent imaging (BLI) and for T cell persistence by flow cytometry and qPCR at day 30 post-T cell injection.

FIG. 112: PBMCs from 2 donors were activated and transduced with retrovirus encoding the CD123 iMC+CARζ-T vector. Six days post-transduction, T cells were seeded onto 96-well plates at 1:10 E:T ratios with THP1-GFP.Fluc cells or HPAC-RFP cells in the presence of 0, 0.1, or 1 nM rimiducid and placed in the IncuCyte to monitor the kinetics THP1-GFP.Fluc or HPAC-RFP growth. (A & B) Two days post-seeding, culture supernatants from a duplicate plate were analyzed for IL-6 and IL-2 production by ELISA. (C) Total green fluorescence intensity of THP1-GFP.Fluc and (D) number of HPAC-RFP cells per well were analyzed using the basic analyzer software for the IncuCyte at day 7.

FIG. 113. PBMCs from 4 donors were activated and co-transduced with retroviruses encoding the CD123 iMC+CARζ-T and RFP vectors. Ten days post-transduction, T cells were seeded onto 96-well plates at 1:1 E:T ratios with THP1-GFP.Fluc cells in the presence of 0 or 1 nM rimiducid and placed in the IncuCyte to monitor the kinetics THP1-GFP.Fluc and T cell-RFP growth. (A) Two days post-seeding, culture supernatants from a duplicate plate were analyzed for IL-2 production by ELISA. (B) On day 7, cells were analyzed for the number of THP1-GFP.Fluc and (C) T cell-RFP remained in the coculture by flow cytometry. (D) Time course monitor of THP1-GFP.Fluc green fluorescence and (E) T cell-RFP red fluorescence analyzed using the IncuCyte for a total of 7 days.

FIG. 114. (A) PBMCs were activated and transduced with retrovirus including the CD123 iMC-CARζ vector. Twelve days after transduction, CAR expression was determined using anti-Q-bend10 antibody before injection into mice. (B) NSG mice were engrafted with 1×10⁶ THP1-GFP.Fluc cells i.v. for 7 days followed by infusion of 2.5×106 non-transduced (NT) or CD123 iMC-CARζ cells i.v. Rimiducid or placebo were given i.p. on days 0 and 15 after T cell infusion at 1 mg/kg. (C) THP1-GFP.Fluc growth was measured using IVIS bioluminescence. (D, E) On day 30, mice were sacrificed and spleens were analyzed for the presence of CAR-T cells by flow cytometry and vector copy number assay.

FIG. 115: (A) NSG mice were engrafted with 1×106 THP1-GFP.Fluc cells i.v. for 7 days followed by treatment with 10e6 NT T cells or various doses of CD123 iMC+CARζ-T cells i.v. Rimiducid or placebo were given i.p. on days 0 and 15 after T cell infusion at 1 mg/kg. (B) On day 29, mice were sacrificed and spleens were analyzed for the presence of CAR-T cells by vector copy number assay.

An iMC-CARζ platform comprising a ligand-dependent activation switch and a proliferation-deficient first generation CAR, efficiently eradicated CD123⁺ leukemic cells when costimulation is provided by systemic rimiducid administration. Deprivation of iMC costimulation resulted in reduction of CAR-T levels, providing a user-controlled system for managing persistence and safety of CD123-specific CAR-T cells.

### Example 32: Inducible MyD88/CD40 Enhances Proliferation and Survival of Tumor-Specific TCR-Modified T Cells and Improves Anti-Tumor Efficacy in Myeloma

Provided is an example of the use of one of the two molecular switches, iMC, in the context of tumor-specific recombinant TCR-expressing T cells.

Cancer immunotherapy using T cells engineered to express tumor antigen-specific TCRs has shown promise in the clinic; however, durable responses have been limited by poor T cell expansion and persistence in vivo. In addition, downregulation of MHC class I on tumor cells diminishes T cell recognition, leading to reduced therapeutic efficacy.

Inducible MyD88/CD40 (iMC) is a rimiducid (AP1903)-dependent costimulatory molecule that enhances DC activation1 and T cell proliferation and survival. PRAME (PReferentially expressed Antigen in MElanoma) is a cancer testis (CT) antigen that is overexpressed in a number of cancers, including melanoma, sarcoma, AML, CML, neuroblastoma, breast, lung, head and neck cancers, but not in normal tissues. Bob1 (also known as OCA-B, OBF1 or POU2AF1) is a B cell-specific transcriptional co-activator that is highly expressed in CD19⁺ B cells, ALL, CLL, MCL and multiple myeloma (MM).

FIG. 116 is a schematic of a “Costimulation on demand” system, controlled using an inducible costimulatory polypeptide (iMC) to better regulate potent T cell therapy. T cell activation and proliferation is TCR- and iMC-dependent. Maximal tumor-directed cytotoxicity, as well as T cell persistence in vivo, requires synergistic signals from a tumor-specific TCR and rimiducid-activated iMC.

FIG. 117: (A-C) Retroviral vectors expressing PRAME TCR (Amir, et al.), or a vector encoding a PRAME TCR, an iMC polypeptide, and a surface marker, (D) PRAME TCR recognition of SLL-peptide pulsed T2 cells synergizes with rimiducid-dependent iMC signals for maximal IL-2 secretion.

FIG. 118: (A) Trans-well assay set-up. (B) Cytokines secreted by transduced T cells in the top well upregulate HLA class I on the surface of SK-N-SH neuroblastoma cells in an antigen-independent, but iMC- and rimiducid-dependent manner.

FIG. 119(A) iMC-PRAME TCR-mediated cytotoxicity against HLA-A2⁺PRAME⁺ U2OS osteosarcoma is rimiducid-independent (B) Signals from the PRAME TCR synergize with rimiducid-driven iMC costimulation, resulting in maximal IL-2 secretion. The Go156 TCR is a negative control TCR.

FIG. 120: (A) iMC-Bob-1 TCR-mediated cytotoxicity against HLA-B7⁺Bob-1⁺ U266 multiple myeloma is rimiducid-independent. (B) Signals from the Bob-1 TCR synergize with rimiducid-driven iMC costimulation, resulting in maximal IL-2 secretion. Go156 TCR is a negative control TCR.

FIG. 121: (A) NSG mice were engrafted with 1×10⁶ luciferase-expressing U266 myeloma cells and treated with 1×107 non-transduced, PRAME TCR- or iMC-PRAME TCR-transduced T cells on day 13. Starting on day 14, five of the mice that received iMC-PRAME-transduced T cells received 5 mg/kg rimiducid i.p. weekly until day 38. (B) Tumor growth was measured by bioluminescence imaging. (C,D) Mice were sacrificed on day 94 and the spleens were analyzed for persistence of human T cells. iMC costimulation significantly increased the number of Vβ1⁺CD8⁺ T cells (C) but not the number of Vβ1⁺CD4⁺ T cells (D).

Rimiducid-driven iMC activation provides potent costimulatory signals in transduced T cells, synergizing with signals from exogenous PRAME- or Bob1-specific TCRs, leading to enhanced T cell proliferation/survival and improved anti-tumor efficacy both in vitro and in vivo.

iMC activation upregulates HLA class I levels on tumor targets, which should lead to improved cytotoxicity via both engineered and endogenous T cells.

### REFERENCES

Narayanan P et al., A composite MyD88/CD40 switch synergistically activates mouse and human dendritic cells for enhanced antitumor efficacy. J Clin Invest. (2011) 121:1524.
Amir A L et al., PRAME-specific Allo-HLA-restricted T cells with potent antitumor reactivity useful for therapeutic T-cell receptor gene transfer. Clin Cancer Res (2011) 17:5615.

### Example 33: Representative Embodiments

Provided hereafter are examples of certain embodiments of the technology.

A1. A nucleic acid comprising a promoter operably linked to a first polynucleotide coding for a first chimeric polypeptide, wherein:
the first chimeric polypeptide comprises a first multimerizing region that binds to a first ligand;
the first multimerizing region comprises a first ligand binding unit and a second ligand binding unit;
the first ligand is a multimeric ligand comprising a first portion and a second portion;
the first ligand binding unit binds to the first portion of the first ligand and does not bind significantly to the second portion of the first ligand; and
the second ligand binding unit binds to the second portion of the first ligand and does not bind significantly to the first portion of the first ligand.

A2. The nucleic acid of embodiment A1, wherein the first chimeric polypeptide comprises a pro-apoptotic polypeptide region.

A2.1. The nucleic acid of embodiment A2, wherein the first multimerizing region is amino terminal to the pro-apoptotic polypeptide region.

A2.2. The nucleic acid of embodiment A2, wherein the first multimerizing region is carboxyl terminal to the pro-apoptotic polypeptide region.

A3. The nucleic acid of embodiment A1, wherein the first chimeric polypeptide comprises
a) a MyD88 polypeptide region or a truncated MyD88 polypeptide region lacking the TIR domain; and
b) a CD40 cytoplasmic polypeptide region lacking the CD40 extracellular domain.

A4. The nucleic acid of any one of embodiments A1-A3, comprising a second polynucleotide coding for a second chimeric polypeptide, wherein:

the promoter is operably linked to the second polynucleotide;
the second chimeric polypeptide comprises a second multimerizing region that binds to a second ligand;
the second multimerizing region comprises a third ligand binding unit;
the second ligand is a multimeric ligand comprising a third portion; and
the third ligand binding unit binds to the third portion of the second ligand and does not bind significantly to the second portion of the first ligand.

A5. The nucleic acid of embodiment A4, wherein the first portion of the first ligand and the third portion of the second ligand are the same.

A6. The nucleic acid of embodiment A4, wherein the first portion of the first ligand and the third portion of the second ligand are different.

A7. The nucleic acid of embodiment A4, wherein the first ligand binding unit of the first multimerizing region and the third ligand binding unit of the second multimerizing region are the same.

A8. The nucleic acid of embodiment A4, wherein the first ligand binding unit of the first multimerizing region and the third ligand binding unit of the second multimerizing region are different.

A9. The nucleic acid of any one of embodiments A4-A8, wherein the second chimeric polypeptide comprises a pro-apoptotic polypeptide region and the first chimeric polypeptide does not comprise the pro-apoptotic polypeptide region.

A10. The nucleic acid of embodiment A9, wherein the second chimeric polypeptide comprises
a) a MyD88 polypeptide region or a truncated MyD88 polypeptide region lacking the TIR domain; and
b) a CD40 cytoplasmic polypeptide region lacking the CD40 extracellular domain
wherein the second multimerizing region of the second chimeric polypeptide comprises at least two third binding units.

A11. The nucleic acid of any one of embodiments A1-A8, wherein the second chimeric polypeptide comprises an MC polypeptide, wherein the MC polypeptide comprises
a) a MyD88 polypeptide region or a truncated MyD88 polypeptide region lacking the TIR domain; and
b) a CD40 cytoplasmic polypeptide region lacking the CD40 extracellular domain and the first chimeric polypeptide does not comprise the MC polypeptide.

A12. The nucleic acid of embodiment A1l, wherein the second chimeric polypeptide comprises a pro-apoptotic polypeptide region.

A13. The nucleic acid of any one of embodiments A1-A12, wherein the first ligand binding unit is FKBP12 or an FKBP12 variant.

A14. The nucleic acid of embodiment A13, wherein the first ligand binding unit is FKBP12.

A15. The nucleic acid of any one of embodiments A1-A14, wherein the second ligand binding unit is FRB or an FRB variant.

A16. The nucleic acid of embodiment A15, wherein the second ligand binding unit is FRBL.

A17. The nucleic acid of any one of embodiments A1-A16, wherein the third ligand binding unit is FKBPv36.

A18. The nucleic acid of embodiment A17, wherein the first ligand binding unit is not FKBPv36.

A19. The nucleic acid of any one of embodiments A1-A18, wherein the first ligand is rapamycin or a rapalog.

A20. The nucleic acid of any one of embodiments A1-A19, wherein the second ligand is AP1903.

A21. The nucleic acid of any one of embodiments A1-A20, wherein the third ligand binding unit binds to the third portion of the second ligand with 100× more affinity than the first ligand binding unit binds to the third portion of the second ligand.

A22. The nucleic acid of embodiment any one of embodiments A1-A20, wherein the third ligand binding unit binds to the third portion of the second ligand with 500× more affinity than the first ligand binding unit binds to the third portion of the second ligand.

A23. The nucleic acid of any one of embodiments A1-A20, wherein the third ligand binding unit binds to the third portion of the second ligand with 1000× more affinity than the first ligand binding unit binds to the third portion of the second ligand.

A24. The nucleic acid of any one of embodiments A1-A23, further comprising a polynucleotide that encodes a chimeric antigen receptor.

A25. The nucleic acid of embodiment A24, wherein the chimeric antigen receptor comprises (i) a transmembrane region, (ii) a T cell activation molecule, and (iii) an antigen recognition moiety.

A26. The nucleic acid of any one of embodiments A1-A23, further comprising a polynucleotide that encodes a chimeric T cell receptor.

A27. A modified cell comprising a nucleic acid of any one of embodiments A1-A26.

A28. A modified cell, comprising a first polynucleotide coding for a first chimeric polypeptide, wherein:

the first chimeric polypeptide comprises a first multimerizing region that binds to a first ligand;
the first multimerizing region comprises a first ligand binding unit and a second ligand binding unit;
the first ligand is a multimeric ligand comprising a first portion and a second portion;
the first ligand binding unit binds to the first portion of the first ligand and does not bind significantly to the second portion of the first ligand; and
the second ligand binding unit binds to the second portion of the first ligand and does not bind significantly to the first portion of the first ligand.

A29. The modified cell of embodiment A28, wherein the first chimeric polypeptide comprises a pro-apoptotic polypeptide region.

A30. The modified cell of embodiment A28, wherein the first chimeric polypeptide comprises
a) a MyD88 polypeptide region or a truncated MyD88 polypeptide region lacking the TIR domain; and
b) a CD40 cytoplasmic polypeptide region lacking the CD40 extracellular domain.

A31. The modified cell of any one of embodiments A28-A30, comprising a second polynucleotide coding for a second chimeric polypeptide, wherein:

the second chimeric polypeptide comprises a second multimerizing region that binds to a second ligand;
the second multimerizing region comprises a third ligand binding unit;
the second ligand is a multimeric ligand comprising a third portion; and
the third ligand binding unit binds to the third portion of the second ligand and does not bind significantly to the second portion of the first ligand.

A32. The modified cell of embodiment A31, wherein the first portion of the first ligand and the third portion of the second ligand are the same.

A33. The modified cell of embodiment A31, wherein the first portion of the first ligand and the third portion of the second ligand are different.

A34. The modified cell of embodiment A31, wherein the first ligand binding unit of the first multimerizing region and the third ligand binding unit of the second multimerizing region are the same.

A35. The modified cell of embodiment A31, wherein the first ligand binding unit of the first multimerizing region and the third ligand binding unit of the second multimerizing region are different.

A36. The modified cell of any one of embodiments A31-A35, wherein the second chimeric polypeptide comprises a pro-apoptotic polypeptide region and the first chimeric polypeptide does not comprise the pro-apoptotic polypeptide region.

A37. The modified cell of embodiment A36, wherein the second chimeric polypeptide comprises
a) a MyD88 polypeptide region or a truncated MyD88 polypeptide region lacking the TIR domain; and
b) a CD40 cytoplasmic polypeptide region lacking the CD40 extracellular domain
wherein the second multimerizing region of the second chimeric polypeptide comprises at least two third binding units.

A38. The modified cell of any one of embodiments A28-A35, wherein the second chimeric polypeptide comprises an MC polypeptide, wherein the MC polypeptide comprises
a) a MyD88 polypeptide region or a truncated MyD88 polypeptide region lacking the TIR domain; and
b) a CD40 cytoplasmic polypeptide region lacking the CD40 extracellular domain and the first chimeric polypeptide does not comprise the MC polypeptide.

A39. The modified cell of embodiment A38, wherein the second chimeric polypeptide comprises a pro-apoptotic polypeptide region.

A40. The modified cell of any one of embodiments A28-A39, wherein the first ligand binding unit is FKBP12 or an FKBP12 variant.

A41. The modified cell of embodiment A40, wherein the first ligand binding unit is FKBP12.

A42. The modified cell of any one of embodiments A28-A41, wherein the second ligand binding unit is FRB or an FRB variant.

A43. The modified cell of embodiment A42, wherein the second ligand binding unit is FRBL.

A44. The modified cell of any one of embodiments A28-A43, wherein the third ligand binding unit is FKBPv36.

A45. The modified cell of embodiment A44, wherein the first ligand binding unit is not FKBPv36.

A46. The modified cell of any one of embodiments A28-A45, wherein the first ligand is rapamycin or a rapalog.

A47. The modified cell of any one of embodiments A28-A46, wherein the second ligand is AP1903.

A48. The modified cell of any one of embodiments A28-A47, wherein the third ligand binding unit binds to the third portion of the second ligand with 100× more affinity than the first ligand binding unit binds to the third portion of the second ligand.

A49. The modified cell of embodiment any one of embodiments A28-A47, wherein the third ligand binding unit binds to the third portion of the second ligand with 500× more affinity than the first ligand binding unit binds to the third portion of the second ligand.

A50. The modified cell of any one of embodiments A28-A47, wherein the third ligand binding unit binds to the third portion of the second ligand with 1000× more affinity than the first ligand binding unit binds to the third portion of the second ligand.

A51. The modified cell of any one of embodiments A28-A50, further comprising a polynucleotide that encodes a chimeric antigen receptor.

A52. The modified cell of embodiment A51, wherein the chimeric antigen receptor comprises (i) a transmembrane region, (ii) a T cell activation molecule, and (iii) an antigen recognition moiety.

A53. The modified cell of any one of embodiments A28-A50, further comprising a polynucleotide that encodes a chimeric T cell receptor.

A54. A modified cell, comprising
a) a first chimeric polypeptide, wherein:
   the first chimeric polypeptide comprises a first multimerizing region that binds to a first ligand;
   the first multimerizing region comprises a first ligand binding unit and a second ligand binding unit;
   the first ligand is a multimeric ligand comprising a first portion and a second portion;
   the first ligand binding unit binds to the first portion of the first ligand and does not bind significantly to the second portion of the first ligand; and
   the second ligand binding unit binds to the second portion of the first ligand and does not bind significantly to the first portion of the first ligand; and
b) a second chimeric polypeptide, wherein:
   the second chimeric polypeptide comprises a second multimerizing region that binds to a second ligand;
   the second multimerizing region comprises a third ligand binding unit;
   the second ligand is a multimeric ligand comprising a third portion; and
   the third ligand binding unit binds to the third portion of the second ligand and does not bind significantly to the second portion of the first ligand.

A55. The modified cell of embodiment A54, comprising a first polynucleotide that encodes the first chimeric polypeptide and a second polynucleotide that encodes the second chimeric polypeptide.

A56. The modified cell of any one of embodiments A28-A55, comprising the first ligand or the second ligand.

A57. A kit or composition comprising nucleic acid comprising a first polynucleotide and a second polynucleotide, wherein
a) the a first polynucleotide encodes a first chimeric polypeptide, wherein:
   the first chimeric polypeptide comprises a first multimerizing region that binds to a first ligand;
   the first multimerizing region comprises a first ligand binding unit and a second ligand binding unit;
   the first ligand is a multimeric ligand comprising a first portion and a second portion;
   the first ligand binding unit binds to the first portion of the first ligand and does not bind significantly to the second portion of the first ligand; and
   the second ligand binding unit binds to the second portion of the first ligand and does not bind significantly to the first portion of the first ligand; and
b) the second polynucleotide encodes a second chimeric polypeptide, wherein the a second chimeric polypeptide, wherein: the second chimeric polypeptide comprises a second multimerizing region that binds to a second ligand;
   the second multimerizing region comprises a third ligand binding unit;
   the second ligand is a multimeric ligand comprising a third portion; and
   the third ligand binding unit binds to the third portion of the second ligand and does not bind significantly to the second portion of the first ligand.

1. A nucleic acid comprising a promoter operably linked to a polynucleotide coding for a chimeric pro-apoptotic polypeptide, wherein the chimeric pro-apoptotic polypeptide comprises
a) a pro-apoptotic polypeptide region;
b) a FRB or FRB variant region; and
c) a FKBP12 polypeptide region.

2. The nucleic acid of embodiment 1, wherein the order of regions (a), (b), and (c), from the amino terminus to the carboxyl terminus of the chimeric pro-apoptotic polypeptide is (c), (b), (a).

3. The nucleic acid of embodiment 1, wherein the order of regions (a), (b), and (c), from the amino terminus to the carboxyl terminus of the chimeric pro-apoptotic polypeptide is (b), (c), (a).

3.1. The nucleic acid of any one of embodiments 2 or 3, wherein (b) and (c) are amino terminal to the pro-apoptotic polypeptide.

3.2. The nucleic acid of any one of embodiments 2 or 3, wherein (b) and (c) are carboxyl terminal to the pro-apoptotic polypeptide.

4. The nucleic acid of any one of embodiments 1 to 3.2, wherein the chimeric pro-apoptotic polypeptide further comprises linker polypeptides between regions (a), (b), and (c).

5. The nucleic acid of any one of embodiments 1-4, further comprising a polynucleotide coding for a marker polypeptide.

6. A polypeptide encoded by a nucleic acid of any one of embodiments 1 to 5.

7. A modified cell transfected or transduced with a nucleic acid of any one of embodiments 1 to 5.

8. A nucleic acid comprising a promoter operably linked to
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 variant regions;
   ii) a MyD88 polypeptide region or a truncated MyD88 polypeptide region lacking the TIR domain; and
   iii) a CD40 cytoplasmic polypeptide region lacking the CD40 extracellular domain.

8.2. A nucleic acid comprising a promoter operably linked to
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 variant regions; and
   ii) a MyD88 polypeptide region or a truncated MyD88 polypeptide region lacking the TIR domain.

9. The nucleic acid of any one of embodiments 8 or 8.5, wherein the FKBP12 variant regions bind to a ligand with at least 100 times more affinity than the ligand binds to the FKBP12 region.

9.1. The nucleic acid of embodiment 8, wherein the FKBP12 variant regions bind to a ligand with at least 500 times more affinity than the ligand binds to the FKBP12 region.

9.2. The nucleic acid of embodiment 8, wherein the FKBP12 variant regions bind to a ligand with at least 1000 times more affinity than the ligand binds to the FKBP12 region.

10. The nucleic acid of embodiment 8, wherein the FKBP12 variant regions are FKBP12v36 regions.

11. A nucleic acid comprising a promoter operably linked to
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 v36 regions;
   ii) a MyD88 polypeptide region or a truncated MyD88 polypeptide region lacking the TIR domain; and
   iii) a CD40 cytoplasmic polypeptide region lacking the CD40 extracellular domain.

12. The nucleic acid of any one of embodiments 8-11, wherein the order of regions (i), (ii), and (iii), from the amino terminus to the carboxyl terminus of the chimeric pro-apoptotic polypeptide is (iii), (ii), (i).

13. The nucleic acid of any one of embodiments 8-11, wherein the order of regions (i), (ii), and (iii), from the amino terminus to the carboxyl terminus of the chimeric pro-apoptotic polypeptide is (ii), (iii), (i).

14. The nucleic acid of any one of embodiments 8 to 13, further comprising linker polypeptides between regions (a), (b), and (c) of the chimeric pro-apoptotic polypeptide.

15. The nucleic acid of any one of embodiments 8-14, wherein the nucleic acid further comprises a polynucleotide encoding a linker polypeptide between the first and second polynucleotides, wherein the linker polypeptide separates the translation products of the first and second polynucleotides during or after translation.

16. The nucleic acid of embodiment 15, wherein the linker polypeptide that separates the translation products of the first and second polynucleotides is a 2A polypeptide.

17. The nucleic acid of any one of embodiments 8-16, wherein the promoter is operably linked to the first polynucleotide and the second polynucleotide.

17.1. The nucleic acid of any one of embodiments 8-17, further comprising a polynucleotide coding for a marker polypeptide.

18. The nucleic acid of any one of embodiments 1-5, or 8-17.1, wherein the promoter is developmentally regulated.

19. The nucleic acid of any one of embodiments 1-5, or 8-17.1, wherein the promoter is tissue-specific.

20. The nucleic acid of any one of embodiments 1-5, or 8-19, wherein the promoter is activated in activated T cells.

21. The nucleic acid of any one of embodiments 8-20, further comprising a third polynucleotide coding for a chimeric antigen receptor.

22. The nucleic acid of embodiment 21, wherein the chimeric antigen receptor comprises (i) a transmembrane region, (ii) a T cell activation molecule, and (iii) an antigen recognition moiety.

23. The nucleic acid of any one of embodiments 8-20, further comprising a third polynucleotide coding for a chimeric T cell receptor.

24. The nucleic acid of any one of embodiments 21-23, further comprising polynucleotides encoding linker polypeptides between the first, second, and third polynucleotides, wherein the linker polypeptide separates the translation products of the first, second, and third polynucleotides during or after translation.

25. The nucleic acid of embodiment 24, wherein the linker polypeptides that separate the translation products of the first, second, and third polynucleotides are 2A polypeptides.

26. A modified cell transduced or transfected with a nucleic acid of any one of embodiments 8-25.

27. A modified cell, comprising
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 variant regions;
   ii) a MyD88 polypeptide region or a truncated MyD88 polypeptide region lacking the TIR domain; and
   iii) a CD40 cytoplasmic polypeptide region lacking the CD40 extracellular domain.

27.2. A modified cell, comprising
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 variant regions; and
   ii) a MyD88 polypeptide region or a truncated MyD88 polypeptide region lacking the TIR domain.

28. The modified cell of any one of embodiments 27 and 27.5, wherein the FKBP12 variant regions bind to a ligand with at least 100 times less affinity than the ligand binds to the FKBP12 region.

29. The modified cell of embodiment 27, wherein the FKBP12 variant regions bind to a ligand with at least 500 times less affinity than the ligand binds to the FKBP12 region.

30. The modified cell of embodiment 27, wherein the FKBP12 variant regions bind to a ligand with at least 1000 times less affinity than the ligand binds to the FKBP12 region.

31. The modified cell of any one of embodiments 27-30, wherein the FKBP12 variant regions are FKBP12v36 regions.

31.1. The modified cell of embodiment 31, wherein the ligand is AP1903.

32. A modified cell, comprising
a) a first polynucleotide encoding a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) a second polynucleotide encoding a chimeric costimulating polypeptide, wherein the chimeric costimulating polypeptide comprises
   i) two FKBP12 v36 regions;
   ii) a MyD88 polypeptide region or a truncated MyD88 polypeptide region lacking the TIR domain; and
   iii) a CD40 cytoplasmic polypeptide region lacking the CD40 extracellular domain.

33. The modified cell of any one of embodiments 27-32, wherein the order of regions (i), (ii), and (iii), from the amino terminus to the carboxyl terminus of the chimeric pro-apoptotic polypeptide is (iii), (ii), (i).

34. The modified cell of any one of embodiments 27-32, wherein the order of regions (i), (ii), and (iii), from the amino terminus to the carboxyl terminus of the chimeric pro-apoptotic polypeptide is (ii), (iii), (i).

35. The modified cell of any one of embodiments 27-34, further comprising linker polypeptides between regions (a), (b), and (c) of the chimeric pro-apoptotic polypeptide.

36. The modified cell of any one of embodiments 26-35, wherein the cell further comprises a chimeric antigen receptor.

37. The modified cell of embodiment 36, wherein the chimeric antigen receptor comprises (i) a transmembrane region, (ii) a T cell activation molecule, and (iii) an antigen recognition moiety.

38. The modified cell of any one of embodiments 26-35, wherein the cell further comprises a chimeric T cell receptor.

39. The modified cell of embodiment 7, or of embodiments A27-A56, wherein the cell is a T cell, tumor infiltrating lymphocyte, NK-T cell, or NK cell.

40. The modified cell of embodiment 7, or of embodiments A27-A56, wherein the cell is a T cell.

41. The modified cell of embodiment 7, or of embodiments A27-A56, wherein the cell is a primary T cell.

42. The modified cell of embodiment 7, or of embodiments A27-A56, wherein the cell is a cytotoxic T cell.

43. The modified cell of embodiment 7, or of embodiments A27-A56, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

44. The modified cell of embodiment 7, or of embodiments A27-A56, wherein the T cell is a helper T cell.

45. The modified cell of any one of embodiments 7, or 39-44, or of embodiments A27-A56, wherein the cell is obtained or prepared from bone marrow.

46. The modified cell of any one of embodiments 7, or 39-44, or of embodiments A27-A56, wherein the cell is obtained or prepared from umbilical cord blood.

47. The modified cell of any one of embodiments 7, or 39-44, or of embodiments A27-A56, wherein the cell is obtained or prepared from peripheral blood.

48. The modified cell of any one of embodiments 7, or 39-44, or of embodiments A27-A56, wherein the cell is obtained or prepared from peripheral blood mononuclear cells.

49. The modified cell of any one of embodiments 7, or 39-48, or of embodiments A27-A56, wherein the cell is a human cell.

50. The modified cell of any one of embodiments 7, or 39-49, or of embodiments A27-A56, wherein the modified cell is transduced or transfected in vivo.

51. The modified cell of any one of embodiments 7, or 39-50, or of embodiments A27-A56, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

52. The modified cell of any one of embodiments 26-38, or of embodiments A27-A56, wherein the cell is a T cell, tumor infiltrating lymphocyte, NK-T cell, or NK cell.

53. The modified cell of any one of embodiments 26-38, or of embodiments A27-A56, wherein the cell is a T cell.

54. The modified cell of any one of embodiments 26-38, or of embodiments A27-A56, wherein the cell is a primary T cell.

55. The modified cell of any one of embodiments 26-38, or of embodiments A27-A56, wherein the cell is a cytotoxic T cell.

56. The modified cell of any one of embodiments 26-38, or of embodiments A27-A56, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

57. The modified cell of any one of embodiments 26-38, or of embodiments A27-A56, wherein the T cell is a helper T cell.

58. The modified cell of any one of embodiments 26-38, or 52-57, or of embodiments A27-A56, wherein the cell is obtained or prepared from bone marrow.

59. The modified cell of any one of embodiments 26-38, or 52-57, or of embodiments A27-A56, wherein the cell is obtained or prepared from umbilical cord blood.

60. The modified cell of any one of embodiments 26-38, or 52-57, or of embodiments A27-A56, wherein the cell is obtained or prepared from peripheral blood.

61. The modified cell of any one of embodiments 26-38, or 52-57, or of embodiments A27-A56, wherein the cell is obtained or prepared from peripheral blood mononuclear cells.

62. The modified cell of any one of embodiments 26-38, or 52-61, or of embodiments A27-A56, wherein the cell is a human cell.

63. The modified cell of any one of embodiments 26-38, or 52-62, or of embodiments A27-A56, wherein the modified cell is transduced or transfected in vivo.

64. The modified cell of any one of embodiments 26-38, or 52-63, or of embodiments A27-A56, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

64.1. A modified cell, comprising
a) a first chimeric pro-apop the chimeric pro-apoptotic polypeptide comprises
   i) a pro-apoptotic polypeptide region;
   ii) a FRB or FRB variant region; and
   iii) a FKBP12 polypeptide region; and
b) a chimeric costimulating polypeptide, wherein the chimeric costimulating polypeptide comprises
   i) two FKBP12 variant regions;
   ii) a MyD88 polypeptide region or a truncated MyD88 polypeptide region lacking the TIR domain; and
   iii) a CD40 cytoplasmic polypeptide region lacking the CD40 extracellular domain.

64.2. A modified cell, comprising
a) a first chimeric pro-apop the chimeric pro-apoptotic polypeptide comprises
   i) a pro-apoptotic polypeptide region;
   ii) a FRB or FRB variant region; and
   iii) a FKBP12 polypeptide region; and
b) a chimeric costimulating polypeptide, wherein the chimeric costimulating polypeptide comprises
   i) two FKBP12 variant regions; and
   ii) a MyD88 polypeptide region or a truncated MyD88 polypeptide region lacking the TIR domain.

64.3. The modified cell of claim 64.1 or 64.2, comprising a first polynucleotide that encodes the first chimeric polypeptide and a second polynucleotide that encodes the second polypeptide.

64.4. A kit or composition comprising nucleic acid comprising a first polynucleotide and a second polynucleotide, wherein
a) the first polynucleotide encodes a chimeric pro-apoptotic polypeptide, wherein the chimeric pro-apoptotic polypeptide comprises
   i) a pro-apoptotic polypeptide region;
   ii) a FRB or FRB variant region; and
   iii) a FKBP12 polypeptide region; and
b) the second polynucleotide encodes a chimeric costimulating polypeptide, wherein the chimeric costimulating polypeptide comprises
   i) two FKBP12 variant regions;
   ii) a MyD88 polypeptide region or a truncated MyD88 polypeptide region lacking the TIR domain; and
   iii) a CD40 cytoplasmic polypeptide region lacking the CD40 extracellular domain.

65. The nucleic acid or cell of any one of embodiments 5, 7, or 17.1-64, or of embodiments A1-A56, wherein the marker polypeptide is a ΔCD19 polypeptide.

66. The nucleic acid or cell of any one of embodiments 1-9, 12-31.1, or 33-65, wherein the FKBP12 variant region has an amino acid substitution at position 36 selected from the group consisting of valine, leucine, isoleuceine and alanine.

67. The nucleic acid or cell of embodiment 66, wherein FKBP variant region is an FKBP12v36 region.

68. The nucleic acid or cell of any one of embodiments 1-67, wherein the FRB variant region is selected from the group consisting of KLW (T2098L), KTF (W2101F), and KLF (T2098L, W2101F).

69. The nucleic acid or cell of any one of embodiments 1-67, wherein the FRB variant region is FRBL

70. The nucleic acid or cell of any one of embodiments 1-69, wherein the FRB variant region binds to a rapalog selected from the group consisting of S-o,p-dimethoxyphenyl (DMOP)-rapamycin, R-Isopropoxyrapamycin, and S-Butanesulfonamidorap.

71. The nucleic acid or cell of any one of embodiments 1-70, wherein the pro-apoptotic polypeptide is selected from the group consisting of caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-xL, Bcl-2, RIPK3, and RIPK1-RHIM.

72. The nucleic acid or cell of any one of embodiments 1-71, wherein the pro-apoptotic polypeptide is a caspase polypeptide.

73. The nucleic acid or cell of embodiment 84, wherein the pro-apoptotic polypeptide is a Caspase-9 polypeptide.

74. The nucleic acid of cell of embodiment 73, wherein the Caspase-9 polypeptide lacks the CARD domain.

75. The nucleic acid or cell of any one of embodiments 73 or 74, wherein the caspase polypeptide comprises the amino acid sequence of SEQ ID NO: 300.

76. The nucleic acid or cell of any one of embodiments 73 or 74, wherein the caspase polypeptide is a modified Caspase-9 polypeptide comprising an amino acid substitution selected from the group consisting of the catalytically active caspase variants in Tables 5 or 6.

77. The nucleic acid or cell of embodiment 76, wherein the caspase polypeptide is a modified Caspase-9 polypeptide comprising an amino acid sequence selected from the group consisting of D330A, D330E, and N405Q.

78. The nucleic acid or cell of any one of embodiments 8-38, or 52-77, wherein the truncated MyD88 polypeptide has the amino acid sequence of SEQ ID NO: 214, or a functional fragment thereof.

79. The nucleic acid or cell of any one of embodiments 8-38, or 52-77, wherein the MyD88 polypeptide has the amino acid sequence of SEQ ID NO: 282, or a functional fragment thereof.

80. The nucleic acid or cell of any one of embodiments 8-38, or 52-77, wherein the cytoplasmic CD40 polypeptide has the amino acid sequence of SEQ ID NO: 216, or a functional fragment thereof.

81. The nucleic acid or cell of any one of embodiments 23, 26, 38, or 52-64, wherein the T cell receptor binds to an antigenic polypeptide selected from the group consisting of PRAME, Bob-1, and NY-ESO-1.

82. The nucleic acid or cell of any one of embodiments 22, 26, 37, or 52-80, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

83. The nucleic acid or cell of any one of embodiments 22, 26, 37, 52-80, or 82, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.

84. The nucleic acid or cell of any one of embodiments 22, 26, 37, 52-80, or 82-83, wherein the antigen recognition moiety is a single chain variable fragment.

85. The nucleic acid or cell of any one of embodiments 22, 26, 37, 52-80, or 82-84, wherein the transmembrane region is a CD8 transmembrane region.

86. The nucleic acid of any one of embodiments 1-5, 8-25, or 65-85, wherein the nucleic acid is contained within a viral vector.

87. The nucleic acid of embodiment 86, wherein the viral vector is selected from the group consisting of retroviral vector, murine leukemia virus vector, SFG vector, adenoviral vector, lentiviral vector, adeno-associated virus (AAV), Herpes virus, and Vaccinia virus.

88. The nucleic acid of any one of embodiments 1-5, 8-25, or 65-87, wherein the nucleic acid is prepared or in a vector designed for electroporation, sonoporation, or biolistics, or is attached to or incorporated in chemical lipids, polymers, inorganic nanoparticles, or polyplexes.

89. The nucleic acid of any one of embodiments 1-5 8-25, or 65-85, wherein the nucleic acid is contained within a plasmid.

90. The nucleic acid or cell of any one of embodiments 1-89, comprising a polynucleotide coding for a polypeptide provided in the tables of Examples 23 or 25.

91. The nucleic acid or cell of any one of embodiments 1-89, comprising a polynucleotide coding for a polypeptide provided in the tables of Examples 23 or 25 selected from group consisting of FKBPv36, FpK′, FpK, Fv, Fv′, FKBPpK′, FKBPpK″, and FKBPpK′″.

92. The nucleic acid or cell of any one of embodiments 1-89, comprising a polynucleotide coding for a polypeptide provided in the tables of Examples 23 or 25 selected from group consisting of FRP5-VL, FRP5-VH, FMC63-VL, and FMC63-VH.

93. The nucleic acid or cell of any one of embodiments 1-89, comprising a polynucleotide coding for FRP5-VL and FRP5-VH.

94. The nucleic acid or cell of any one of embodiments 1-89, comprising a polynucleotide coding for FMC63-VL and FMC63-VH.

95. The nucleic acid or cell of any one of embodiments 1-89, comprising a polynucleotide coding for a polypeptide provided in the tables of Examples 23 or 25 selected from group consisting of MyD88L and MyD88.

96. The nucleic acid or cell of any one of embodiments 1-89, comprising a polynucleotide coding for a ΔCaspase-9 polypeptide provided in the tables of Examples 23 or 25.

97. The nucleic acid or cell of any one of embodiments 1-89, comprising a polynucleotide coding for a ΔCD18 polypeptide provided in the tables of Examples 23 or 25.

98. The nucleic acid or cell of any one of embodiments 1-89, comprising a polynucleotide coding for a hCD40 polypeptide provided in the tables of Examples 23 or 25.

99. The nucleic acid or cell of any one of embodiments 1-89, comprising a polynucleotide coding for a CD3 zeta polypeptide provided in the tables of Examples 23 or 25.

100. Reserved.

101. A method of stimulating an immune response in a subject, comprising:
a) transplanting modified cells of any one of embodiments A27-A56, 26-38, or 52-85 into the subject,
   and
b) after (a), administering an effective amount of a ligand that binds to the FKBP12 variant region of the chimeric costimulating polypeptide to stimulate a cell mediated immune response.

102. A method of administering a ligand to a human subject who has undergone cell therapy using modified cells, comprising administering a ligand that binds to the FKBP variant region of the chimeric costimulating polypeptide to the human subject, wherein the modified cells comprise modified cells of any one of embodiments A27-A56, 26-38, or 52-85.

103. A method of controlling activity of transplanted modified cells in a subject, comprising:
a) transplanting a modified cell of any one of embodiments A27-A56, 26-38, or 52-85; and
b) after (a), administering an effective amount of a ligand that binds to the FKBP12 variant region of the chimeric costimulating polypeptide to stimulate the activity of the transplanted modified cells.

104. A method for treating a subject having a disease or condition associated with an elevated expression of a target antigen expressed by a target cell, comprising

(a) transplanting an effective amount of modified cells into the subject; wherein the modified cells comprise a modified cell of any one of embodiments A27-A56, 26-38, or 52-85, wherein the modified cell comprises a chimeric antigen receptor comprising an antigen recognition moiety that binds to the target antigen, and
(b) after a), administering an effective amount of a ligand that binds to the FKBP12 variant region of the chimeric costimulating polypeptide to reduce the number or concentration of target antigen or target cells in the subject.

105. The method of embodiment 104, wherein the target antigen is a tumor antigen.

106. A method for treating a subject having a disease or condition associated with an elevated expression of a target antigen expressed by a target cell, comprising

(a) administering to the subject an effective amount of modified cells, wherein the modified cells comprise a modified cell of any one of embodiments A27-A56, 26-38, or 52-85, wherein the modified cell comprises a chimeric T cell receptor that recognizes and binds to the target antigen, and
(b) after a), administering an effective amount of a ligand that binds to the FKBP12 variant region of the chimeric costimulating polypeptide to reduce the number or concentration of target antigen or target cells in the subject.

107. A method for reducing the size of a tumor in a subject, comprising
a) administering a modified cell of any one of embodiments A27-A56, 26-38, or 52-85 to the subject, wherein the cell comprises a chimeric antigen receptor comprising an antigen recognition moiety that binds to an antigen on the tumor; and
b) after a), administering an effective amount of a ligand that binds to the FKBP12 variant region of the chimeric costimulating polypeptide to reduce the size of the tumor in the subject.

108. The method of any one of embodiments 104-107, comprising measuring the number or concentration of target cells in a first sample obtained from the subject before administering second ligand, measuring the number or concentration of target cells in a second sample obtained from the subject after administering the ligand, and determining an increase or decrease of the number or concentration of target cells in the second sample compared to the number or concentration of target cells in the first sample.

109. The method of embodiment 108, wherein the concentration of target cells in the second sample is decreased compared to the concentration of target cells in the first sample.

110. The method of embodiment 108, wherein the concentration of target cells in the second sample is increased compared to the concentration of target cells in the first sample.

111. The method of any one of embodiments 101-110, wherein the subject has received a stem cell transplant before or at the same time as administration of the modified cells.

112. The method of any one of embodiments 101-111, wherein at least 1×10⁶ transduced or transfected modified cells are administered to the subject.

113. The method of any one of embodiments 101-111, wherein at least 1×10⁷ transduced or transfected modified cells are administered to the subject.

114. The method of any one of embodiments 101-111, wherein at least 1×10⁸ modified cells are administered to the subject.

114.1. The method of any one of embodiments 101-114, wherein the FKBP12 variant region is FKBP12v36 and the ligand that binds to the FKBP12 variant region is AP1903.

115. A method of controlling survival of transplanted modified cells in a subject, comprising
a) transplanting modified cells of any one of embodiments A27-A56, 26-38, 52-64, or 65-85 into the subject, and
b) after (a), administering to the subject rapamycin or a rapalog that binds to the FRB or FRB variant region of the chimeric pro-apoptotic polypeptide in an amount effective to kill less than 30% of the modified cells that express the chimeric pro-apoptotic polypeptide.

116. The method of any one of embodiments 101-114.1, further comprising after (b), administering to the subject rapamycin or a rapalog that binds to the FRB variant region of the chimeric pro-apoptotic polypeptide in an amount effective to kill less than 30% of the modified cells that express the chimeric pro-apoptotic polypeptide.

116.1. the method of embodiment 116, wherein the rapamycin or rapalog is administered in an amount effective to kill at least 30% of the modified cells that express the chimeric pro-apoptotic polypeptide.

117. The method of any one of embodiments 115 or 116, wherein the rapamycin or rapalog is administered in an amount effective to kill less than 40% of the modified cells that express the chimeric pro-apoptotic polypeptide.

118. The method of any one of embodiments 115 or 116, wherein the rapamycin or rapalog is administered in an amount effective to kill less than 50% of the modified cells that express the chimeric pro-apoptotic polypeptide.

119. The method of any one of embodiments 115 or 116, wherein the rapamycin or rapalog is administered in an amount effective to kill less than 60% of the modified cells that express the chimeric pro-apoptotic polypeptide.

120. The method of any one of embodiments 115 or 116, wherein the rapamycin or rapalog is administered in an amount effective to kill less than 70% of the modified cells that express the chimeric pro-apoptotic polypeptide.

121. The method of any one of embodiments 115 or 116, wherein the rapamycin or rapalog is administered in an amount effective to kill less than 90% of the modified cells that express the chimeric pro-apoptotic polypeptide.

122. The method of any one of embodiments 115 or 116, wherein the rapamycin or rapalog is administered in an amount effective to kill at least 90% of the modified cells that express the chimeric pro-apoptotic polypeptide.

123. The method of any one of embodiments 115 or 116, wherein the rapamycin or rapalog is administered in an amount effective to kill at least 95% of the modified cells that express the chimeric pro-apoptotic polypeptide.

124. The method of any one of embodiments 115-116, wherein the chimeric pro-apoptotic polypeptide comprises a FRB L region.

125. The method of any one of embodiments 101-114.1, wherein more than one dose of the ligand is administered to the subject.

126. The method of any one of embodiments 115-125, wherein more than one dose of the rapamycin or rapalog is administered to the subject.

127. The method of any one of embodiments 101-125, further comprising identifying a presence or absence of a condition in the subject that requires the removal of the modified cells from the subject; and administering rapamycin or a rapalog, maintaining a subsequent dosage of rapamycin or the rapalog, or adjusting a subsequent dosage of the rapamycin or the rapalog to the subject based on the presence or absence of the condition identified in the subject.

128. The method of any one of embodiments 101-125, further comprising receiving information comprising presence or absence of a condition in the subject that requires the removal of the modified cells from the subject; and administering the rapamycin or rapalog, maintaining a subsequent dosage of rapamycin or the rapalog, or adjusting a subsequent dosage of rapamycin or the rapalog to the subject based on the presence or absence of the condition identified in the subject.

129. The method of any one of embodiments 101-125, further comprising identifying a presence or absence of a condition in the subject that requires the removal of the modified cells from the subject; and transmitting the presence, absence or stage of the condition identified in the subject to a decision maker who administers rapamycin or the rapalog, maintains a subsequent dosage of the rapamycin or the rapalog, or adjusts a subsequent dosage of the rapamycin or the rapalog administered to the subject based on the presence, absence or stage of the condition identified in the subject.

130. The method of any one of embodiments 101-125, further comprising identifying a presence or absence of a condition in the subject that requires the removal of the modified cells from the subject; and transmitting an indication to administer the rapamycin or the rapalog, maintain a subsequent dosage of the rapamycin or the rapalog, or adjust a subsequent dosage of the rapamycin or the rapalog administered to the subject based on the presence, absence or stage of the condition identified in the subject.

131. The method of any one of embodiments 101-130, wherein the subject has cancer.

132. The method of any one of embodiments 101-131, wherein the modified cell is delivered to a tumor bed.

133. The method of any one of embodiments 131 or 132, wherein the cancer is present in the blood or bone marrow of the subject.

134. The method of any one of embodiments 101-130, wherein the subject has a blood or bone marrow disease.

135. The method of any one of embodiments 101-130, wherein the subject has been diagnosed with sickle cell anemia or metachromatic leukodystrophy.

136. The method of any one of embodiments 101-130, wherein the patient has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.

137. The method of any one of embodiments 101-130, wherein the patient has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

138. A method for expressing a chimeric pro-apoptotic polypeptide, wherein the chimeric pro-apoptotic polypeptide comprises
a) a pro-apoptotic polypeptide region;
b) a FRB or FRB variant region; and
c) a FKBP12 polypeptide region,
   comprising contacting a nucleic acid of any one of embodiments 1-6 with a cell under conditions in which the nucleic acid is incorporated into the cell, whereby the cell expresses the first and second chimeric polypeptides from the incorporated nucleic acid.

139. The method of embodiment 138, wherein the nucleic acid is contacted with the cell ex vivo.

140. The method of embodiment 138, wherein the nucleic acid is contacted with the cell in vivo.

141-200. Reserved.

201. A nucleic acid comprising a promoter operably linked to a polynucleotide coding for a chimeric costimulating polypeptide wherein the chimeric costimulating polypeptide comprises
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region.

202. The nucleic acid of embodiment 201, wherein the order of regions (a), (b), and (c), from the amino terminus to the carboxyl terminus of the chimeric costimulating polypeptide is (c), (b), (a).

203. The nucleic acid of embodiment 201, wherein the order of regions (a), (b), and (c), from the amino terminus to the carboxyl terminus of the chimeric costimulating polypeptide is (b), (c), (a).

204. The nucleic acid of any one of embodiments 201 to 203, further comprising linker polypeptides between regions (a), (b), and (c) of the chimeric costimulating polypeptide.

205. The nucleic acid of any one of embodiments 201-204, further comprising a polynucleotide coding for a marker polypeptide.

206. A polypeptide encoded by a nucleic acid of any one of embodiments 201 to 205.

207. A modified cell transfected or transduced with a nucleic acid of any one of embodiments 201 to 205.

208. A nucleic acid comprising a promoter operably linked to a first polynucleotide encoding a chimeric costimulating polypeptide, comprising
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region; and
   a second polynucleotide encoding a chimeric pro-apoptotic polypeptide, wherein the chimeric pro-apoptotic polypeptide comprises
d) two FKBP12 variant regions; and
e) a pro-apoptotic polypeptide region.

208.2. A nucleic acid comprising a promoter operably linked to a first polynucleotide encoding a chimeric costimulating polypeptide, comprising
a) a costimulating polypeptide region comprising a MyD88 polypeptide region or a truncated MyD88 polypeptide region lacking the TIR domain;
b) a FRB or FRB variant region; and
c) a FKBP12 polypeptide region; and
   a second polynucleotide encoding a chimeric pro-apoptotic polypeptide, wherein the chimeric pro-apoptotic polypeptide comprises
d) two FKBP12 variant regions; and
e) a pro-apoptotic polypeptide region.

209. The nucleic acid of embodiment 208, wherein the FKBP12 variant regions bind to a ligand with at least 100 times less affinity than the ligand binds to the FKBP12 region.

209.1. The nucleic acid of embodiment 208, wherein the FKBP12 variant regions bind to a ligand with at least 500 times less affinity than the ligand binds to the FKBP12 region.

209.2. The nucleic acid of embodiment 208, wherein the FKBP12 variant regions bind to a ligand with at least 1000 times less affinity than the ligand binds to the FKBP12 region.

210. The nucleic acid of embodiment 208, wherein the FKBP12 variant regions are FKBP12v36 regions.

211. A nucleic acid comprising a promoter operably linked to a first polynucleotide encoding a chimeric costimulating polypeptide, comprising
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region; and
   a second polynucleotide encoding a chimeric pro-apoptotic polypeptide, wherein the chimeric pro-apoptotic polypeptide comprises
d) two FKBP12v36 regions; and
e) a pro-apoptotic polypeptide region.

212. The nucleic acid of any one of embodiments 208-211, wherein the order of regions (a), (b), and (c), from the amino terminus to the carboxyl terminus of the chimeric costimulating polypeptide is (c), (b), (a).

213. The nucleic acid of any one of embodiments 208-211, wherein the order of regions (a), (b), and (c), from the amino terminus to the carboxyl terminus of the chimeric costimulating polypeptide is (b), (c), (a).

214. The nucleic acid of any one of embodiments 208 to 213, further comprising linker polypeptides between regions (a), (b), and (c) of the chimeric costimulating polypeptide.

215. The nucleic acid of any one of embodiments 208-214, wherein the nucleic acid further comprises a polynucleotide encoding a linker polypeptide between the first and second polynucleotides, wherein the linker polypeptide separates the translation products of the first and second polynucleotides during or after translation.

216. The nucleic acid of embodiment 215, wherein the linker polypeptide that separates the translation products of the first and second polynucleotides is a 2A polypeptide.

217. The nucleic acid of any one of embodiments 208-216, wherein the promoter is operably linked to the first polynucleotide and the second polynucleotide.

217.1. The nucleic acid of any one of embodiments 208-217, further comprising a polynucleotide coding for a marker polypeptide.

218. The nucleic acid of any one of embodiments 201-205, or 208-217.1, wherein the promoter is developmentally regulated.

219. The nucleic acid of any one of embodiments 201-205, or 208-217.1, wherein the promoter is tissue-specific.

220. The nucleic acid of any one of embodiments 201-205, or 208-219, wherein the promoter is activated in activated T cells.

221. The nucleic acid of any one of embodiments 208-220, further comprising a third polynucleotide coding for a chimeric antigen receptor.

222. The nucleic acid of embodiment 21, wherein the chimeric antigen receptor comprises (i) a transmembrane region, (ii) a T cell activation molecule, and (iii) an antigen recognition moiety.

223. The nucleic acid of any one of embodiments 208-220, further comprising a third polynucleotide coding for a chimeric T cell receptor.

224. The nucleic acid of any one of embodiments 221-223, further comprising polynucleotides encoding linker polypeptides between the first, second, and third polynucleotides, wherein the linker polypeptides separate the translation products of the first and second polynucleotides during or after translation.

225. The nucleic acid of embodiment 224, wherein the linker polypeptide that separates the translation products of the first, second, and third polynucleotides is a 2A polypeptide.

226. A modified cell transduced or transfected with a nucleic acid of any one of embodiments 208-225.

227. A modified cell, comprising

a first polynucleotide encoding a chimeric costimulating polypeptide, comprising
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region; and
   a second polynucleotide encoding a chimeric pro-apoptotic polypeptide, wherein the chimeric pro-apoptotic polypeptide comprises
d) two FKBP12 variant regions;
e) a pro-apoptotic polypeptide region.

228. The modified cell of embodiment 227, wherein the FKBP12 variant regions bind to a ligand with at least 100 times less affinity than the ligand binds to the FKBP12 region.

229. The modified cell of embodiment 227, wherein the FKBP12 variant regions bind to a ligand with at least 500 times less affinity than the ligand binds to the FKBP12 region.

230. The modified cell of embodiment 227, wherein the FKBP12 variant regions bind to a ligand with at least 1000 times less affinity than the ligand binds to the FKBP12 region.

231. The modified cell of any one of embodiments 227-230, wherein the FKBP12 variant regions are FKBP12v36 regions.

231.1. The modified cell of embodiment 231, wherein the ligand is AP1903.

232. A modified cell, comprising a first polynucleotide encoding a chimeric costimulating polypeptide, comprising
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region; and
   a second polynucleotide encoding a chimeric pro-apoptotic polypeptide, wherein the chimeric pro-apoptotic polypeptide comprises
d) two FKBP12 v36 regions;
e) a pro-apoptotic polypeptide region.

233. The modified cell of any one of embodiment 227-232, wherein the order of regions (a), (b), and (c), from the amino terminus to the carboxyl terminus of the chimeric costimulating polypeptide is (c), (b), (a).

234. The modified cell of any one of embodiment 227-232, wherein the order of regions (a), (b), and (c), from the amino terminus to the carboxyl terminus of the chimeric costimulating polypeptide is (b), (c), (a).

235. The modified cell of any one of embodiment 227-235, further comprising linker polypeptides between regions (a), (b), and (c) of the chimeric costimulating polypeptide.

236. The modified cell of any one of embodiment 226-234, wherein the cell further comprises a chimeric antigen receptor.

237. The modified cell of embodiment 236, wherein the chimeric antigen receptor comprises (i) a transmembrane region, (ii) a T cell activation molecule, and (iii) an antigen recognition moiety.

238. The modified cell of any one of embodiment 226-235, wherein the cell further comprises a chimeric T cell receptor.

239. The modified cell of embodiment 207, wherein the cell is a T cell, tumor infiltrating lymphocyte, NK-T cell, or NK cell.

240. The modified cell of embodiment 207, wherein the cell is a T cell.

241. The modified cell of embodiment 207, wherein the cell is a primary T cell.

242. The modified cell of embodiment 207, wherein the cell is a cytotoxic T cell.

243. The modified cell of embodiment 207, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

244. The modified cell of embodiment 207, wherein the T cell is a helper T cell.

245. The modified cell of any one of embodiments 207, or 239-244, wherein the cell is obtained or prepared from bone marrow.

246. The modified cell of any one of embodiments 207, or 239-244, wherein the cell is obtained or prepared from umbilical cord blood.

247. The modified cell of any one of embodiments 207, or 239-244, wherein the cell is obtained or prepared from peripheral blood.

248. The modified cell of any one of embodiments 207, or 239-244, wherein the cell is obtained or prepared from peripheral blood mononuclear cells.

249. The modified cell of any one of embodiments 207, or 239-248, wherein the cell is a human cell.

250. The modified cell of any one of embodiments 207, or 239-249, wherein the modified cell is transduced or transfected in vivo.

251. The modified cell of any one of embodiments 207, or 239-250, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

252. The modified cell of any one of embodiment 226-238, wherein the cell is a T cell, tumor infiltrating lymphocyte, NK-T cell, or NK cell.

253. The modified cell of any one of embodiment 226-238, wherein the cell is a T cell.

254. The modified cell of any one of embodiment 226-238, wherein the cell is a primary T cell.

255. The modified cell of any one of embodiment 226-238, wherein the cell is a cytotoxic T cell.

256. The modified cell of any one of embodiment 226-238, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

257. The modified cell of any one of embodiment 226-238, wherein the T cell is a helper T cell.

258. The modified cell of any one of embodiment 226-238, or 252-257, wherein the cell is obtained or prepared from bone marrow.

259. The modified cell of any one of embodiment 226-238, or 252-257, wherein the cell is obtained or prepared from umbilical cord blood.

260. The modified cell of any one of embodiment 226-238, or 252-257, wherein the cell is obtained or prepared from peripheral blood.

261. The modified cell of any one of embodiment 226-238, or 252-257, wherein the cell is obtained or prepared from peripheral blood mononuclear cells.

262. The modified cell of any one of embodiment 226-238, or 252-261, wherein the cell is a human cell.

263. The modified cell of any one of embodiment 226-238, or 252-262, wherein the modified cell is transduced or transfected in vivo.

264. The modified cell of any one of embodiment 226-238, or 252-263, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

264.1. A modified cell, comprising
a) a first polynucleotide encoding a chimeric costimulating polypeptide, comprising a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
      a FRB or FRB variant region; and
      a FKBP12 polypeptide region; and
b) a second polynucleotide encoding a chimeric pro-apoptotic polypeptide, wherein the chimeric pro-apoptotic polypeptide comprises two FKBP12 variant regions; and a pro-apoptotic polypeptide region.

264.2. The modified cell of claim 264.1, comprising a first polynucleotide that encodes the first chimeric polypeptide and a second polynucleotide that encodes the second polypeptide.

264.3. A kit or composition comprising nucleic acid comprising a first polynucleotide and a second polynucleotide, wherein the first polynucleotide encodes a chimeric costimulating polypeptide, comprising
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region; and
   the second polynucleotide encodes a chimeric pro-apoptotic polypeptide, wherein the chimeric pro-apoptotic polypeptide comprises
a) two FKBP12 variant regions; and
b) a pro-apoptotic polypeptide region.

265. The nucleic acid or cell of any one of embodiments 205, 207, or 217.1-264, wherein the marker polypeptide is a ΔCD19 polypeptide.

266. The nucleic acid or cell of any one of embodiments 102-109, 212-231.1, or 233-265, wherein the FKBP12 variant region has an amino acid substitution at position 36 selected from the group consisting of valine, leucine, isoleuceine and alanine.

267. The nucleic acid or cell of embodiment 266, wherein FKBP variant region is an FKBP12v36 region.

268. The nucleic acid or cell of any one of embodiments 201-267, wherein the FRB variant region is selected from the group consisting of KLW (T2098L), KTF (W2101F), and KLF (T2098L, W2101F).

269. The nucleic acid or cell of any one of embodiments 201-267, wherein the FRB variant region is FRBL

270. The nucleic acid or cell of any one of embodiments 201-269, wherein the FRB variant region binds to a rapalog selected from the group consisting of S-o,p-dimethoxyphenyl (DMOP)-rapamycin, R-Isopropoxyrapamycin, and S-Butanesulfonamidorap.

271. The nucleic acid or cell of any one of embodiments 201-270, wherein the pro-apoptotic polypeptide is selected from the group consisting of caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-xL, Bcl-2, RIPK3, and RIPK1-RHIM.

272. The nucleic acid or cell of any one of embodiments 208-271, wherein the pro-apoptotic polypeptide is a caspase polypeptide.

273. The nucleic acid or cell of embodiment 284, wherein the pro-apoptotic polypeptide is a Caspase-9 polypeptide.

274. The nucleic acid of cell of embodiment 273, wherein the Caspase-9 polypeptide lacks the CARD domain.

275. The nucleic acid or cell of any one of embodiments 273 or 274, wherein the caspase polypeptide comprises the amino acid sequence of SEQ ID NO: 300.

276. The nucleic acid or cell of any one of embodiments 273 or 274, wherein the caspase polypeptide is a modified Caspase-9 polypeptide comprising an amino acid substitution selected from the group consisting of the catalytically active caspase variants in Tables 5 or 6.

277. The nucleic acid or cell of embodiment 276, wherein the caspase polypeptide is a modified Caspase-9 polypeptide comprising an amino acid sequence selected from the group consisting of D330A, D330E, and N405Q.

278. The nucleic acid or cell of any one of embodiments 201-277, wherein the truncated MyD88 polypeptide has the amino acid sequence of SEQ ID NO: 214, or a functional fragment thereof.

279. The nucleic acid or cell of any one of embodiments 201-277, wherein the MyD88 polypeptide has the amino acid sequence of SEQ ID NO: 282, or a functional fragment thereof.

280. The nucleic acid or cell of any one of embodiments 201-277, wherein the cytoplasmic CD40 polypeptide has the amino acid sequence of SEQ ID NO: 216, or a functional fragment thereof.

281. The nucleic acid or cell of any one of embodiment 223, 226, 38, or 252-280, wherein the T cell receptor binds to an antigenic polypeptide selected from the group consisting of PRAME, Bob-1, and NP-ESO-1.

282. The nucleic acid or cell of any one of embodiment 222, 226, 237, or 252-280, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1, ROR1, Mesothelin, GD2, CD123, Muc16, CD33, CD38, and CD44v6.

283. The nucleic acid or cell of any one of embodiment 222, 226, 237, 252-280, or 282, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.

284. The nucleic acid or cell of any one of embodiment 222, 226, 237, 252-280, or 282-283, wherein the antigen recognition moiety is a single chain variable fragment.

285. The nucleic acid or cell of any one of embodiment 222, 226, 237, 252-280, or 282-284, wherein the transmembrane region is a CD8 transmembrane region.

286. The nucleic acid of any one of embodiments 201-205, 208-225, or 265-285, wherein the nucleic acid is contained within a viral vector.

287. The nucleic acid of embodiment 286, wherein the viral vector is selected from the group consisting of retroviral vector, murine leukemia virus vector, SFG vector, adenoviral vector, lentiviral vector, adeno-associated virus (AAV), Herpes virus, and Vaccinia virus.

288. The nucleic acid of any one of embodiments 201-205, 208-225, or 265-287, wherein the nucleic acid is prepared or in a vector designed for electroporation, sonoporation, or biolistics, or is attached to or incorporated in chemical lipids, polymers, inorganic nanoparticles, or polyplexes.

289. The nucleic acid of any one of embodiments 201-205, 208-225, or 265-285, wherein the nucleic acid is contained within a plasmid.

290. The nucleic acid or cell of any one of embodiments 201-289, comprising a polynucleotide coding for a polypeptide provided in the tables of Examples 23 or 25.

291. The nucleic acid or cell of any one of embodiments 201-289, comprising a polynucleotide coding for a polypeptide provided in the tables of Examples 23 or 25 selected from group consisting of FKBPv36, FpK′, FpK, Fv, Fv′, FKBPpK′, FKBPpK″, and FKBPpK′″.

292. The nucleic acid or cell of any one of embodiments 201-289, comprising a polynucleotide coding for a polypeptide provided in the tables of Examples 23 or 25 selected from group consisting of FRP5-VL, FRP5-VH, FMC63-VL, and FMC63-VH.

293. The nucleic acid or cell of any one of embodiments 201-289, comprising a polynucleotide coding for FRP5-VL and FRP5-VH.

294. The nucleic acid or cell of any one of embodiments 201-289, comprising a polynucleotide coding for FMC63-VL and FMC63-VH.

295. The nucleic acid or cell of any one of embodiments 201-289, comprising a polynucleotide coding for a polypeptide provided in the tables of Examples 23 or 25 selected from group consisting of MyD88L and MyD88.

296. The nucleic acid or cell of any one of embodiments 201-289, comprising a polynucleotide coding for a ΔCaspase-9 polypeptide provided in the tables of Examples 23 or 25.

297. The nucleic acid or cell of any one of embodiments 201-289, comprising a polynucleotide coding for a ΔCD18 polypeptide provided in the tables of Examples 23 or 25.

298. The nucleic acid or cell of any one of embodiments 201-289, comprising a polynucleotide coding for a hCD40 polypeptide provided in the tables of Examples 23 or 25.

299. The nucleic acid or cell of any one of embodiments 201-289, comprising a polynucleotide coding for a CD3zeta polypeptide provided in the tables of Examples 23 or 25.

300. Reserved.

301. A method of stimulating an immune response in a subject, comprising:
a) transplanting modified cells of any one of embodiments 226-238, 252-264, or 265-285 into the subject,
   and
b) after (a), administering an effective amount of a rapamycin or a rapalog that binds to the FRB or FRB variant region of the chimeric stimulating polypeptide to stimulate a cell mediated immune response.

302. A method of administering a ligand to a human subject who has undergone cell therapy using modified cells, comprising administering rapamycin or a rapalog to the human subject, wherein the modified cells comprise modified cells of any one of embodiments 226-238, 252-264, or 265-285.

303. A method of controlling activity of transplanted modified cells in a subject, comprising:
a) transplanting a modified cell of any one of embodiments 226-238, or 252-285; and
b) after (a), administering an effective amount of rapamycin or a rapalog that binds to the FRB or FRB variant region of the chimeric stimulating polypeptide to stimulate the activity of the transplanted modified cells.

304. A method for treating a subject having a disease or condition associated with an elevated expression of a target antigen expressed by a target cell, comprising
a) transplanting an effective amount of modified cells into the subject; wherein the modified cells comprise a modified cell of any one of embodiments 226-238, or 252-285, wherein the modified cell comprises a chimeric antigen receptor comprising an antigen recognition moiety that binds to the target antigen, and
b) after a), administering an effective amount of rapamycin or a rapalog that binds to the FRB or FRB variant region of the chimeric stimulating polypeptide to reduce the number or concentration of target antigen or target cells in the subject.

305. The method of embodiment 304, wherein the target antigen is a tumor antigen.

306. A method for treating a subject having a disease or condition associated with an elevated expression of a target antigen expressed by a target cell, comprising
a) administering to the subject an effective amount of modified cells, wherein the modified cells comprise a modified cell of any one of embodiments 226-238, or 252-285, wherein the modified cell comprises a chimeric T cell receptor that recognizes and binds to the target antigen, and
b) after a), administering an effective amount of rapamycin or a rapalog that binds to the FRB or FRB variant region of the chimeric stimulating polypeptide to reduce the number or concentration of target antigen or target cells in the subject.

307. A method for reducing the size of a tumor in a subject, comprising
a) administering a modified cell of any one of embodiments 226-238, or 252-285 to the subject, wherein the cell comprises a chimeric antigen receptor comprising an antigen recognition moiety that binds to an antigen on the tumor; and
b) after a), administering an effective amount of rapamycin or a rapalog that binds to the FRB or FRB variant region of the chimeric stimulating polypeptide to reduce the size of the tumor in the subject.

308. The method of any one of embodiments 304-307, comprising measuring the number or concentration of target cells in a first sample obtained from the subject before administering second ligand, measuring the number or concentration of target cells in a second sample obtained from the subject after administering the ligand, and determining an increase or decrease of the number or concentration of target cells in the second sample compared to the number or concentration of target cells in the first sample.

309. The method of embodiment 308, wherein the concentration of target cells in the second sample is decreased compared to the concentration of target cells in the first sample.

310. The method of embodiment 308, wherein the concentration of target cells in the second sample is increased compared to the concentration of target cells in the first sample.

311. The method of any one of embodiments 301-310, wherein the subject has received a stem cell transplant before or at the same time as administration of the modified cells.

312. The method of any one of embodiments 301-311, wherein at least 1×10⁶ transduced or transfected modified cells are administered to the subject.

313. The method of any one of embodiments 301-311, wherein at least 1×10⁷ transduced or transfected modified cells are administered to the subject.

314. The method of any one of embodiments 301-311, wherein at least 1×10⁸ modified cells are administered to the subject.

314.1. The method of any one of embodiments 301-314, wherein the FKBP12 variant region is FKBP12v36 and the ligand that binds to the FKBP12 variant region is AP1903.

315. A method of controlling survival of transplanted modified cells in a subject, comprising
a) transplanting modified cells of any one of embodiments 226-238, or 252-285 into the subject,
   and
b) after (a), administering to the a ligand that binds to the FKBP12 variant region of the chimeric pro-apoptotic polypeptide in an amount effective to kill less than 30% of the modified cells that express the chimeric pro-apoptotic polypeptide.

316. The method of any one of embodiments 301-314.1, further comprising after (b), administering to the subject a ligand that binds to the FKBP12 variant region of the chimeric pro-apoptotic polypeptide in an amount effective to kill less than 30% of the modified cells that express the chimeric pro-apoptotic polypeptide.

317. The method of any one of embodiments 315 or 316, wherein the a ligand that binds to the FKBP12 variant region is administered in an amount effective to kill less than 40% of the modified cells that express the chimeric pro-apoptotic polypeptide.

318. The method of any one of embodiments 315 or 316, wherein the a ligand that binds to the FKBP12 variant region is administered in an amount effective to kill less than 50% of the modified cells that express the chimeric pro-apoptotic polypeptide.

319. The method of any one of embodiments 315 or 316, wherein the a ligand that binds to the FKBP12 variant region is administered in an amount effective to kill less than 60% of the modified cells that express the chimeric pro-apoptotic polypeptide.

320. The method of any one of embodiments 315 or 316, wherein the a ligand that binds to the FKBP12 variant region is administered in an amount effective to kill less than 70% of the modified cells that express the chimeric pro-apoptotic polypeptide.

321. The method of any one of embodiments 315 or 316, wherein the a ligand that binds to the FKBP12 variant region is administered in an amount effective to kill less than 90% of the modified cells that express the chimeric pro-apoptotic polypeptide.

322. The method of any one of embodiments 315 or 316, wherein the a ligand that binds to the FKBP12 variant region is administered in an amount effective to kill at least 90% of the modified cells that express the chimeric pro-apoptotic polypeptide.

323. The method of any one of embodiments 315 or 316, wherein the a ligand that binds to the FKBP12 variant region is administered in an amount effective to kill at least 95% of the modified cells that express the chimeric pro-apoptotic polypeptide.

324. The method of any one of embodiments 315-316, wherein the chimeric costimulating polypeptide comprises a FRB L region.

325. The method of any one of embodiments 301-314.1, wherein more than one dose of the ligand is administered to the subject.

326. The method of any one of embodiments 315-325, wherein more than one dose of the a ligand that binds to the FKBP12 variant region is administered to the subject.

327. The method of any one of embodiments 301-325, further comprising identifying a presence or absence of a condition in the subject that requires the removal of the modified cells from the subject; and administering a ligand that binds to the FKBP12 variant region, maintaining a subsequent dosage of the ligand, or adjusting a subsequent dosage of the ligand to the subject based on the presence or absence of the condition identified in the subject.

328. The method of any one of embodiments 301-325, further comprising receiving information comprising presence or absence of a condition in the subject that requires the removal of the modified cells from the subject; and administering the a ligand that binds to the FKBP12 variant region, maintaining a subsequent dosage of the ligand, or adjusting a subsequent dosage of the ligand to the subject based on the presence or absence of the condition identified in the subject.

329. The method of any one of embodiments 301-325, further comprising identifying a presence or absence of a condition in the subject that requires the removal of the modified cells from the subject; and transmitting the presence, absence or stage of the condition identified in the subject to a decision maker who administers a ligand that binds to the FKBP12 variant region, maintains a subsequent dosage of the ligand, or adjusts a subsequent dosage of the ligand administered to the subject based on the presence, absence or stage of the condition identified in the subject.

330. The method of any one of embodiments 301-325, further comprising identifying a presence or absence of a condition in the subject that requires the removal of the modified cells from the subject; and transmitting an indication to administer the a ligand that binds to the FKBP12 variant region, maintain a subsequent dosage of the ligand, or adjust a subsequent dosage of the ligand administered to the subject based on the presence, absence or stage of the condition identified in the subject.

331. The method of any one of embodiments 301-330, wherein the subject has cancer.

332. The method of any one of embodiments 301-331, wherein the modified cell is delivered to a tumor bed.

333. The method of any one of embodiments 331 or 332, wherein the cancer is present in the blood or bone marrow of the subject.

334. The method of any one of embodiments 301-330, wherein the subject has a blood or bone marrow disease.

335. The method of any one of embodiments 301-330, wherein the subject has been diagnosed with sickle cell anemia or metachromatic leukodystrophy.

336. The method of any one of embodiments 301-330, wherein the patient has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.

337. The method of any one of embodiments 301-330, wherein the patient has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

338. A method for expressing a chimeric costimulating polypeptide wherein the chimeric costimulating polypeptide comprises
a) a costimulating polypeptide region comprising
   i) a MyD88 polypeptide region or a truncated MyD88 polypeptide region lacking the TIR domain; and
   ii) a CD40 cytoplasmic polypeptide region lacking the CD40 extracellular domain;
b) a FRB or FRB variant region; and
c) a FKBP12 polypeptide region.
   comprising contacting a nucleic acid of any one of embodiments 301-306 with a cell under conditions in which the nucleic acid is incorporated into the cell, whereby the cell expresses the first and second chimeric polypeptides from the incorporated nucleic acid.

339. The method of embodiment 338, wherein the nucleic acid is contacted with the cell ex vivo.

340. The method of embodiment 338, wherein the nucleic acid is contacted with the cell in vivo.

### Example 34: Additional Representative Embodiments

1. A modified cell, comprising
a) a first polynucleotide encoding a chimeric pro-apoptotic polypeptide, wherein the chimeric pro-apoptotic polypeptide comprises
   i) a pro-apoptotic polypeptide region;
   ii) a FKBP12-Rapamycin-Binding (FRB) domain polypeptide, or FRB variant polypeptide region; and
   iii) a FKBP12 or FKBP12 variant polypeptide region (FKBP12v); and
b) a second polynucleotide encoding a chimeric costimulating polypeptide, wherein the chimeric costimulating polypeptide comprises two FKBP12 variant polypeptide regions and
   i) a MyD88 polypeptide region or a truncated MyD88 polypeptide region lacking the TIR domain; or
   ii) a MyD88 polypeptide region or a truncated MyD88 polypeptide region lacking the TIR domain, and a CD40 cytoplasmic polypeptide region lacking the CD40 extracellular domain.

2. The modified cell of claim 1, wherein the chimeric costimulating polypeptide comprises two FKBP12 variant polypeptide regions and a truncated MyD88 polypeptide region lacking the TIR domain.

3. The modified cell of claim 1, wherein the chimeric costimulating polypeptide comprises two FKBP12 variant polypeptide regions, a truncated MyD88 polypeptide region lacking the TIR domain, and a CD40 cytoplasmic polypeptide region lacking the CD40 extracellular domain.

4. The modified cell of any of claims 1-3, wherein the chimeric pro-apoptotic polypeptide comprises (i) a pro-apoptotic polypeptide region, (ii) a FRB or FRB variant polypeptide region, and (iii) a FKBP12 polypeptide region.

5. The modified cell of any one of claims 1-5, wherein the cell further comprises a third polynucleotide encoding a heterologous protein.

6. The modified cell of claim 6, wherein the heterologous protein is a chimeric antigen receptor.

7. The modified cell of claim 7, wherein the heterologous protein is a recombinant T cell receptor.

8. A nucleic acid comprising a promoter operably linked to
a) a first polynucleotide encoding a chimeric pro-apoptotic polypeptide, wherein the chimeric pro-apoptotic polypeptide comprises
   i) a pro-apoptotic polypeptide region;
   ii) a FKBP12-Rapamycin-Binding (FRB) domain polypeptide, or FRB variant polypeptide region; and
   iii) a FKBP12 or FKBP12 variant polypeptide region (FKBP12v); and
b) a second polynucleotide encoding a chimeric costimulating polypeptide, wherein the chimeric costimulating polypeptide comprises two FKBP12 variant polypeptide regions and
   i) a MyD88 polypeptide region or a truncated MyD88 polypeptide region lacking the TIR domain; or
   ii) a MyD88 polypeptide region or a truncated MyD88 polypeptide region lacking the TIR domain, and a CD40 cytoplasmic polypeptide region lacking the CD40 extracellular domain.

9. The nucleic acid of claim 8, wherein the chimeric pro-apoptotic polypeptide comprises a pro-apoptotic polypeptide region, a FRB or FRB variant polypeptide region, and a FKBP12 polypeptide region.

10. The nucleic acid of any one of claims 8-9, wherein the chimeric costimulating polypeptide comprises a MyD88 polypeptide region or a truncated MyD88 polypeptide region lacking the TIR domain.

11. The nucleic acid of any one of claims 8-9, wherein the chimeric costimulating polypeptide comprises a truncated MyD88 polypeptide region lacking the TIR domain and a CD40 cytoplasmic polypeptide region lacking the CD40 extracellular domain.

12. The nucleic acid of any one of claims 8-11, wherein the promoter is operably linked to a third polynucleotide, wherein the third polynucleotide encodes a heterologous protein.

13. The nucleic acid of claim 12, wherein the heterologous protein is a chimeric antigen receptor.

14. The nucleic acid of claim 12, wherein the heterologous protein is a recombinant TCR.

15. The nucleic acid of any one of claims 8-14, wherein the nucleic acid further comprises a polynucleotide encoding a linker polypeptide between the first polynucleotide and the second polynucleotide, wherein the linker polypeptide separates the translation products of the first and second polynucleotides during or after translation.

16. The nucleic acid of claim 15, wherein the nucleic acid further comprises a polynucleotide encoding a linker polypeptide between the third polynucleotide and the first or the second polynucleotide, wherein the linker polypeptide separates the translation product of the third polynucleotide from the translation products of the first or second polynucleotides during or after translation.

17. The nucleic acid of any one of claim 15 or 16, wherein the linker polypeptide is a 2A polypeptide.

18. A modified cell transduced or transfected with a nucleic acid of any one of claims 8-17

19. The modified cell or the nucleic acid of any one of claims 1-18, wherein the FRB polypeptide or FRB variant polypeptide region and the FKBP12 polypeptide or FKBP12 variant polypeptide region are amino terminal to the pro-apoptotic polypeptide of the chimeric pro-apoptotic polypeptide.

20. The modified cell or the nucleic acid of claim 19, wherein the FRB polypeptide or FRB variant polypeptide region is amino terminal to the FKBP12 polypeptide or FKBP12 variant polypeptide region.

21. The modified cell or the nucleic acid of claim 19, wherein the FKBP12 polypeptide or FKBP12 variant polypeptide region is amino terminal to the FRB or FRB variant polypeptide region.

22. The modified cell or the nucleic acid of any one of claims 1-21, wherein the FKBP12 variant polypeptide region binds to a ligand with at least 100 times more affinity than the ligand binds to the FKBP12 polypeptide region.

23. The modified cell or the nucleic acid of any one of claims 1-21, wherein the FKBP12 variant polypeptide region binds to a ligand with at least 500 times more affinity than the ligand binds to the FKBP12 polypeptide region.

24. The modified cell or the nucleic acid of any one of claims 1-21, wherein the FKBP12 variant polypeptide region binds to a ligand with at least 1000 times more affinity than the ligand binds to the wild type FKBP12 polypeptide region.

25. The modified cell or the nucleic acid of any one of claims 1-24, wherein the FKBP12 variant polypeptide comprises an amino acid substitution at amino acid residue 36.

26. The modified cell or the nucleic acid of claim 25, wherein the amino acid substitution at position 36 selected from the group consisting of valine, leucine, isoleuceine and alanine.

27. The modified cell or the nucleic acid of any one of claims 1-21, wherein the FKBP12 variant polypeptide region is a FKBP12v36 polypeptide region.

28. The modified cell or the nucleic acid of any one of claims 22-24, wherein the ligand is rimiducid.

29. The modified cell or the nucleic acid of any one of claim 224, wherein the ligand is AP20187 or AP1510.

30. The modified cell or the nucleic acid of any one of claims 1-29, wherein the FRB variant polypeptide binds to a C7 rapalog.

31. The modified cell or the nucleic acid of any one of claims 1-30, wherein the FRB variant polypeptide comprises an amino acid substitution at position T2098 or W2101.

32. The modified cell or the nucleic acid of any one of claims 1-31, wherein the FRB variant polypeptide region is selected from the group consisting of KLW (T2098L)(FRBL), KTF (W2101F), and KLF (T2098L, W2101F).

33. The modified cell or the nucleic acid of any one of claims 1-32, wherein the FRB variant polypeptide region is FRBL.

34. The modified cell of any one of claims 1-33, wherein the FRB variant polypeptide region binds to a rapalog selected from the group consisting of S-o,p-dimethoxyphenyl (DMOP)-rapamycin, R-Isopropoxyrapamycin, C7-Isobutyloxyrapamycin, and S-Butanesulfonamidorap.

35. The modified cell or the nucleic acid of any one of claims 1-34, wherein the cell or the nucleic acid comprises a polynucleotide that encodes a chimeric antigen receptor, wherein the chimeric antigen receptor comprises (i) a transmembrane region, (ii) a T cell activation molecule, and (iii) an antigen recognition moiety.

36. The modified cell or the nucleic acid of claim 33, wherein the T cell activation molecule is selected from the group consisting of an ITAM-containing, Signal 1 conferring molecule, a Syk polypeptide, a ZAP70 polypeptide, a CD3ζ polypeptide, and an Fc epsilon receptor gamma (FcεR1γ) subunit polypeptide.

37. The modified cell or the nucleic acid of claim 33, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.

38. The modified cell or the nucleic acid of any one of claims 35-371, wherein the antigen recognition moiety is a single chain variable fragment.

39. The modified cell or the nucleic acid of any one of claims 35-38, wherein the transmembrane region is a CD8 transmembrane region.

40. The modified cell or the nucleic acid of any one of claims 35-39, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

41. The modified cell or the nucleic acid of any one of claims 35-40 wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

42. The modified cell of any one of claims 1-34, wherein the cell comprises a polynucleotide encoding a recombinant T cell receptor, wherein the recombinant T cell receptor binds to an antigenic polypeptide selected from the group consisting of PRAME, Bob-1, and NY-ESO-1.

43. The modified cell or the nucleic acid of any one of claims 1-42, wherein the pro-apoptotic polypeptide is selected from the group consisting of Caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-xL, Bcl-2, RIPK3, and RIPK1-RHIM.

44. The modified cell or the nucleic acid of any one of claims 1-43, wherein the pro-apoptotic polypeptide is a caspase polypeptide.

45. The modified cell or the nucleic acid of claim 44, wherein the pro-apoptotic polypeptide is a Caspase-9 polypeptide.

46. The nucleic acid of cell of claim 45, wherein the Caspase-9 polypeptide lacks the CARD domain.

47. The modified cell or the nucleic acid of any one of claim 45 or 46, wherein the caspase polypeptide comprises the amino acid sequence of SEQ ID NO: 300.

48. The modified cell or the nucleic acid of any one of claims 44-47, wherein the caspase polypeptide is a modified Caspase-9 polypeptide comprising an amino acid substitution selected from the group consisting of the catalytically active caspase variants in Tables 5 or 6.

49. The modified cell or the nucleic acid of claim 48, wherein the caspase polypeptide is a modified Caspase-9 polypeptide comprising an amino acid sequence selected from the group consisting of D330A, D330E, and N405Q.

50. The modified cell or the nucleic acid of any one of claims 1-49, wherein the truncated MyD88 polypeptide has the amino acid sequence of SEQ ID NO: 214 or 305, or a functional fragment thereof.

51. The modified cell or the nucleic acid of any one of claims 1-49, wherein the MyD88 polypeptide has the amino acid sequence of SEQ ID NO: 282, or a functional fragment thereof.

52. The modified cell or the nucleic acid of any one of claims 1-51, wherein the cytoplasmic CD40 polypeptide has the amino acid sequence of SEQ ID NO: 216, or a functional fragment thereof.

53. The modified cell of claim 1, wherein,
a) the chimeric pro-apoptotic polypeptide comprises a Caspase-9 polypeptide lacking the CARD domain, a FRBL polypeptide region and a FKBP12 polypeptide region; and
b) the chimeric costimulating polypeptide comprises a truncated MyD88 polypeptide region lacking the TIR domain and two FKBP12v36 polypeptide regions.

54. The modified cell of claim 1, wherein,
a) the chimeric pro-apoptotic polypeptide comprises a Caspase-9 polypeptide lacking the CARD domain, a FRBL polypeptide region and a FKBP12 polypeptide region; and
b) the chimeric costimulating polypeptide comprises a truncated MyD88 polypeptide region lacking the TIR domain, a CD40 cytoplasmic polypeptide region lacking the extracellular domain, and two FKBP12v36 polypeptide regions.

55. The nucleic acid of claim 19, wherein,
a) the chimeric pro-apoptotic polypeptide comprises a Caspase-9 polypeptide lacking the CARD domain, a FRBL polypeptide region and a FKBP12 polypeptide region; and
b) the chimeric costimulating polypeptide comprises a truncated MyD88 polypeptide region lacking the TIR domain and two FKBP12v36 polypeptide regions.

56. The nucleic acid of claim 19, wherein,
a) the chimeric pro-apoptotic polypeptide comprises a Caspase-9 polypeptide lacking the CARD domain, a FRBL polypeptide region and a FKBP12 polypeptide region; and
b) the chimeric costimulating polypeptide comprises a truncated MyD88 polypeptide region lacking the TIR domain, a CD40 cytoplasmic polypeptide region lacking the extracellular domain, and two FKBP12v36 polypeptide regions.

57. The modified cell of any one of claim 1-8, 18, or 19-36, wherein the cell is a T cell, tumor infiltrating lymphocyte, NK-T cell, or NK cell.

58. The modified cell of any one of claim 1-8, 18, or 19-36, wherein the cell is a T cell, NK-T cell, or NK cell.

59. The modified cell of any one of claim 1-8, 18, or 19-36, wherein the cell is a T cell.

60. The modified cell of any one of claim 1-8, 18, or 19-36, wherein the cell is a primary T cell.

61. The modified cell of any one of claim 1-8, 18, or 19-36, wherein the cell is a cytotoxic T cell.

62. The modified cell of any one of claim 1-8, 18, or 19-36, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

63. The modified cell of any one of claim 1-8, 18, or 19-36, wherein the T cell is a helper T cell.

64. The modified cell of any one of claim 1-8, 18, or 19-36, wherein the cell is obtained or prepared from bone marrow.

65. The modified cell of any one claim 1-8, 18, or 19-36, wherein the cell is obtained or prepared from umbilical cord blood.

66. The modified cell of any one of claim 1-8, 18, or 19-36, wherein the cell is obtained or prepared from peripheral blood.

67. The modified cell of any one of claim 1-8, 18, or 19-36, wherein the cell is obtained or prepared from peripheral blood mononuclear cells.

68. The modified cell of any one of claim 1-8, 18, 19-36 or 57-67, wherein the cell is a human cell.

69. The modified cell of any one of claim 1-8, 18, 19-36 or 57-68, wherein the modified cell is transduced or transfected in vivo.

70. The modified cell of any one of claim 1-8, 18, 19-36 or 57-69, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

71. A kit or composition comprising nucleic acid comprising
a) a first polynucleotide encoding a chimeric pro-apoptotic polypeptide, wherein the chimeric pro-apoptotic polypeptide comprises
   i) a pro-apoptotic polypeptide region;
   ii) a FKBP12-Rapamycin-Binding (FRB) domain polypeptide region, or variant thereof; and
   iii) a FKBP12 polypeptide or FKBP12 variant polypeptide region (FKBP12v); and
b) a second polynucleotide encoding a chimeric costimulating polypeptide, wherein the chimeric costimulating polypeptide comprises two FKBP12 variant polypeptide regions and
   i) a MyD88 polypeptide region or a truncated MyD88 polypeptide region lacking the TIR domain; or
   ii) a MyD88 polypeptide region or a truncated MyD88 polypeptide region lacking the TIR domain, and a CD40 cytoplasmic polypeptide region lacking the CD40 extracellular domain.

72. The kit or composition of claim 71, wherein the chimeric pro-apoptotic polypeptide comprises a pro-apoptotic polypeptide region, a FRB or FRB variant polypeptide region, and a FKBP12 polypeptide region.

73. The kit or composition of any one of claims 71-72, wherein the chimeric costimulating polypeptide comprises a MyD88 polypeptide region or a truncated MyD88 polypeptide region lacking the TIR domain.

74. The kit or composition of any one of claims 71-72, wherein the chimeric costimulating polypeptide comprises a truncated MyD88 polypeptide region lacking the TIR domain and a CD40 cytoplasmic polypeptide region lacking the CD40 extracellular domain.

75. The kit or composition of claim 71, wherein the nucleic acid is a nucleic acid of any one of claim 8-17, 19-212, or 55-56.

76. The kit or composition of any one of claims 71-75, further comprising a third polynucleotide, wherein the third polynucleotide encodes a heterologous protein.

77. The kit or composition of 72, wherein the heterologous protein is a chimeric antigen receptor.

78. The kit or composition of claim 72, wherein the heterologous protein is a recombinant TCR.

79. The kit or composition of any one of claims 71-75, comprising a virus, wherein the virus comprises the first and the second polynucleotide.

80. The kit or composition of any one of claims 72-78, comprising a virus, wherein the virus comprises the first, second, and third polynucleotides.

81. The kit or composition of any one of claims 72-78, comprising a virus, wherein the virus comprises the first and third polynucleotides.

82. The kit or composition of any one of claims 72-78, comprising a virus, wherein the virus comprises the second and third polynucleotides.

83. A method for expressing a chimeric pro-apoptotic polypeptide, wherein the chimeric pro-apoptotic polypeptide comprises
a) a pro-apoptotic polypeptide region; a FRB polypeptide or FRB variant polypeptide region; and
b) a FKBP12 polypeptide region,
   comprising contacting a nucleic acid of any one of claim 8-17, 19-52, or 55-56, with a cell under conditions in which the nucleic acid is incorporated into the cell, whereby the cell expresses the chimeric pro-apoptotic polypeptide from the incorporated nucleic acid.

84. The method of claim 83, wherein the cell further expresses a chimeric costimulating polypeptide, wherein the chimeric costimulating polypeptide comprises
a) two FKBP12 variant polypeptide regions; and
b) a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain.

85. The method of any one of claim 83 or 84, wherein the nucleic acid is contacted with the cell ex vivo.

86. The method of any one of claim 83 or 84, wherein the nucleic acid is contacted with the cell in vivo.

87. A method of stimulating an immune response in a subject, comprising:
a) transplanting modified cells of any one of claim 1-8, 18, 19-36, or 57-70 into the subject, and
b) after (a), administering an effective amount of a ligand that binds to the FKBP12 variant polypeptide region of the chimeric costimulating polypeptide to stimulate a cell mediated immune response.

88. A method of administering a ligand to a subject who has undergone cell therapy using modified cells, comprising administering a ligand that binds to the FKBP variant region of the chimeric costimulating polypeptide to the human subject, wherein the modified cells comprise modified cells of any one of claim 1-8, 18, 19-36, or 57-70.

89. A method of controlling activity of transplanted modified cells in a subject, comprising:
a) transplanting a modified cell of any one of claim 1-8, 18, 19-36, or 57-70; and
b) after (a), administering an effective amount of a ligand that binds to the FKBP12 variant polypeptide region of the chimeric costimulating polypeptide to stimulate the activity of the transplanted modified cells.

90. A method for treating a subject having a disease or condition associated with an elevated expression of a target antigen expressed by a target cell, comprising
a) transplanting an effective amount of modified cells into the subject; wherein the modified cells comprise a modified cell of any one of claim 1-8, 18, 19-36, or 57-70, wherein the modified cell comprises a chimeric antigen receptor comprising an antigen recognition moiety that binds to the target antigen, and
b) after a), administering an effective amount of a ligand that binds to the FKBP12 variant polypeptide region of the chimeric costimulating polypeptide to reduce the number or concentration of target antigen or target cells in the subject.

91. The method of claim 90, wherein the target antigen is a tumor antigen.

92. A method for treating a subject having a disease or condition associated with an elevated expression of a target antigen expressed by a target cell, comprising
a) administering to the subject an effective amount of modified cells, wherein the modified cells comprise a modified cell of any one of claim 1-8, 18, 19-36, or 57-70, wherein the modified cell comprises a recombinant T cell receptor that recognizes and binds to the target antigen, and
b) after a), administering an effective amount of a ligand that binds to the FKBP12 variant polypeptide region of the chimeric costimulating polypeptide to reduce the number or concentration of target antigen or target cells in the subject.

93. A method for reducing the size of a tumor in a subject, comprising
a) administering a modified cell of any one of claim 1-8, 18, 19-36, or 57-70 to the subject, wherein the cell comprises a chimeric antigen receptor comprising an antigen recognition moiety that binds to an antigen on the tumor; and
b) after a), administering an effective amount of a ligand that binds to the FKBP12 variant polypeptide region of the chimeric costimulating polypeptide to reduce the size of the tumor in the subject.

94. The method of any one of claims 90-93, comprising measuring the number or concentration of target cells in a first sample obtained from the subject before administering second ligand, measuring the number or concentration of target cells in a second sample obtained from the subject after administering the ligand, and determining an increase or decrease of the number or concentration of target cells in the second sample compared to the number or concentration of target cells in the first sample.

95. The method of claim 94, wherein the concentration of target cells in the second sample is decreased compared to the concentration of target cells in the first sample.

96. The method of claim 94, wherein the concentration of target cells in the second sample is increased compared to the concentration of target cells in the first sample.

97. The method of any one of claims 87-96, wherein the subject has received a stem cell transplant before or at the same time as administration of the modified cells.

98. The method of any one of claims 87-97, wherein at least 1×106 transduced or transfected modified cells are administered to the subject.

99. The method of any one of claims 87-97, wherein at least 1×107 transduced or transfected modified cells are administered to the subject.

100. The method of any one of claims 87-97, wherein at least 1×108 modified cells are administered to the subject.

101. The method of any one of claims 87-100, wherein the FKBP12 variant polypeptide region is FKBP12v36 and the ligand that binds to the FKBP12 variant polypeptide region is AP1903.

102. A method of controlling survival of transplanted modified cells in a subject, comprising
a) transplanting modified cells of any one of claim 1-8, 18, 19-36, or 57-70 into the subject; and
b) after a), administering to the subject rapamycin or a rapalog that binds to the FRB polypeptide or FRB variant polypeptide region of the chimeric pro-apoptotic polypeptide in an amount effective to kill at least 30% of the modified cells that express the chimeric pro-apoptotic polypeptide.

103. The method of any one of claims 87-102, further comprising after b), administering to the subject rapamycin or a rapalog that binds to the FRB variant polypeptide region of the chimeric pro-apoptotic polypeptide in an amount effective to kill at least 30% of the modified cells that express the chimeric pro-apoptotic polypeptide.

104. The method of claim 103, wherein the rapamycin or rapalog is administered in an amount effective to kill at least 40% of the modified cells that express the chimeric pro-apoptotic polypeptide.

105. The method of any one of claim 102 or 103, wherein the rapamycin or rapalog is administered in an amount effective to kill at least 50% of the modified cells that express the chimeric pro-apoptotic polypeptide.

106. The method of any one of claim 102 or 103, wherein the rapamycin or rapalog is administered in an amount effective to kill at least 60% of the modified cells that express the chimeric pro-apoptotic polypeptide.

107. The method of any one of claim 102 or 103, wherein the rapamycin or rapalog is administered in an amount effective to kill at least 70% of the modified cells that express the chimeric pro-apoptotic polypeptide.

108. The method of any one of claim 102 or 103, wherein the rapamycin or rapalog is administered in an amount effective to kill at least 80% of the modified cells that express the chimeric pro-apoptotic polypeptide.

109. The method of any one of claim 102 or 103, wherein the rapamycin or rapalog is administered in an amount effective to kill at least 90% of the modified cells that express the chimeric pro-apoptotic polypeptide.

110. The method of any one of claim 102 or 103, wherein the rapamycin or rapalog is administered in an amount effective to kill at least 95% of the modified cells that express the chimeric pro-apoptotic polypeptide.

111. The method of any one of claim 102 or 103, wherein the rapamycin or rapalog is administered in an amount effective to kill at least 99% of the modified cells that express the chimeric pro-apoptotic polypeptide.

112. The method of any one of claims 102-103, wherein the chimeric pro-apoptotic polypeptide comprises a FRBL region.

113. The method of any one of claims 87-101, wherein more than one dose of the ligand is administered to the subject.

114. The method of any one of claims 102-113, wherein more than one dose of the rapamycin or rapalog is administered to the subject.

115. The method of any one of claims 87-113, further comprising identifying a presence or absence of a condition in the subject that requires the removal of the modified cells from the subject; and administering rapamycin or a rapalog, maintaining a subsequent dosage of rapamycin or the rapalog, or adjusting a subsequent dosage of the rapamycin or the rapalog to the subject based on the presence or absence of the condition identified in the subject.

116. The method of any one of claims 87-113, further comprising receiving information comprising presence or absence of a condition in the subject that requires the removal of the modified cells from the subject; and administering the rapamycin or rapalog, maintaining a subsequent dosage of rapamycin or the rapalog, or adjusting a subsequent dosage of rapamycin or the rapalog to the subject based on the presence or absence of the condition identified in the subject.

117. The method of any one of claims 87-113, further comprising identifying a presence or absence of a condition in the subject that requires the removal of the modified cells from the subject; and transmitting the presence, absence or stage of the condition identified in the subject to a decision maker who administers rapamycin or the rapalog, maintains a subsequent dosage of the rapamycin or the rapalog, or adjusts a subsequent dosage of the rapamycin or the rapalog administered to the subject based on the presence, absence or stage of the condition identified in the subject.

118. The method of any one of claims 87-113, further comprising identifying a presence or absence of a condition in the subject that requires the removal of the modified cells from the subject; and transmitting an indication to administer the rapamycin or the rapalog, maintain a subsequent dosage of the rapamycin or the rapalog, or adjust a subsequent dosage of the rapamycin or the rapalog administered to the subject based on the presence, absence or stage of the condition identified in the subject.

119. The method of any one of claims 87-118, wherein the subject has cancer.

120. The method of any one of claims 87-119, wherein the modified cell is delivered to a tumor bed.

121. The method of any one of claim 119 or 120, wherein the cancer is present in the blood or bone marrow of the subject.

122. The method of any one of claims 87-118, wherein the subject has a blood or bone marrow disease.

123. The method of any one of claims 87-118, wherein the subject has been diagnosed with sickle cell anemia or metachromatic leukodystrophy.

124. The method of any one of claims 87-118, wherein the subject has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.

125. The method of any one of claims 87-118, wherein the patient has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

126. A modified cell comprising
a) a first polynucleotide encoding a chimeric pro-apoptotic polypeptide, wherein the chimeric pro-apoptotic polypeptide comprises
   i) a pro-apoptotic polypeptide region; and
   ii) a FKBP12 variant polypeptide region; and
b) a second polynucleotide encoding a chimeric costimulating polypeptide, wherein the chimeric costimulating polypeptide comprises
   i) a FKBP12-Rapamycin Binding (FRB) domain polypeptide or FRB variant polypeptide region;
   ii) a FKBP12 polypeptide or FKBP12 variant polypeptide region; and
   iii) a MyD88 polypeptide region or a truncated MyD88 polypeptide region lacking the TIR domain, or a MyD88 polypeptide region, or a truncated MyD88 polypeptide region lacking the TIR domain and a CD40 cytoplasmic polypeptide region lacking the CD40 extracellular domain.

127. The modified cell of claim 126, wherein the chimeric costimulating polypeptide comprises a MyD88 polypeptide region or a truncated MyD88 polypeptide region lacking the TIR domain.

128. The modified cell of claim 126, wherein the chimeric costimulating polypeptide comprises a truncated MyD88 polypeptide region lacking the TIR domain and a CD40 cytoplasmic polypeptide region lacking the CD40 extracellular domain.

129. The modified cell of any one of claims 126-128, wherein the cell further comprises a third polynucleotide, wherein the third polynucleotide encodes a heterologous protein.

130. The modified cell of claim 129, wherein the heterologous protein is a chimeric antigen receptor.

131. The modified cell of claim 129, wherein the heterologous protein is a recombinant TCR.

132. A nucleic acid comprising a promoter operably linked to
a) a first polynucleotide encoding a chimeric pro-apoptotic polypeptide, wherein the chimeric pro-apoptotic polypeptide comprises
   i) a pro-apoptotic polypeptide region; and
   ii) a FKBP12 variant polypeptide region; and
b) a second polynucleotide encoding a chimeric costimulating polypeptide, wherein the chimeric costimulating polypeptide comprises
   i) a FKBP12-Rapamycin Binding (FRB) domain polypeptide or FRB variant polypeptide region;
   ii) a FKBP12 polypeptide region; and
   iii) a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain.

133. The nucleic acid of claim 132, wherein the chimeric costimulating polypeptide comprises a MyD88 polypeptide region or a truncated MyD88 polypeptide region lacking the TIR domain.

134. The nucleic acid of claim 132, wherein the chimeric costimulating polypeptide comprises a truncated MyD88 polypeptide region lacking the TIR domain and a CD40 cytoplasmic polypeptide region lacking the CD40 extracellular domain.

135. The nucleic acid of any one of claims 132-134, wherein the promoter is operably linked to a third polynucleotide, wherein the third polynucleotide encodes a heterologous protein.

136. The nucleic acid of claim 135, wherein the heterologous protein is a chimeric antigen receptor.

137. The nucleic acid of claim 135, wherein the heterologous protein is a recombinant TCR.

138. The nucleic acid of any one of claims 132-137, wherein the nucleic acid further comprises a polynucleotide encoding a linker polypeptide between the first polynucleotide and the second polynucleotide, wherein the linker polypeptide separates the translation products of the first and second polynucleotides during or after translation.

139. The nucleic acid of claim 138, wherein the nucleic acid further comprises a polynucleotide encoding a linker polypeptide between the third polynucleotide and the first or the second polynucleotide, wherein the linker polypeptide separates the translation product of the third polynucleotide from the translation products of the first or second polynucleotides during or after translation.

140. The nucleic acid of any one of claim 138 or 139, wherein the linker polypeptide is a 2A polypeptide.

141. A modified cell transduced or transfected with a nucleic acid of any one of claims 132-140

142. The modified cell or the nucleic acid of any one of claims 126-141, wherein the FRB polypeptide or FRB variant polypeptide region and the FKBP12 polypeptide region are amino terminal to the MyD88 polypeptide or truncated MyD88 polypeptide of the chimeric costimulating polypeptide.

143. The modified cell or the nucleic acid of claim 142, wherein the FRB polypeptide or FRB variant polypeptide region is amino terminal to the FKBP12 polypeptide region.

144. The modified cell or the nucleic acid of claim 142, wherein the FKBP12 polypeptide region is amino terminal to the FRB or FRB variant polypeptide region.

145. The modified cell or the nucleic acid of any one of claims 126-144, wherein the FKBP12 variant polypeptide region binds to a ligand with at least 100 times more affinity than the ligand binds to the FKBP12 polypeptide region.

146. The modified cell or the nucleic acid of any one of claims 126-144, wherein the FKBP12 variant polypeptide region binds to a ligand with at least 500 times more affinity than the ligand binds to the FKBP12 polypeptide region.

147. The modified cell or the nucleic acid of any one of claims 126-144, wherein the FKBP12 variant polypeptide region binds to a ligand with at least 1000 times more affinity than the ligand binds to the FKBP12 polypeptide region.

148. The modified cell or the nucleic acid of any one of claims 126-147, wherein the FKBP12 variant polypeptide comprises an amino acid substitution at amino acid residue 36.

149. The modified cell or the nucleic acid of claim 148, wherein the amino acid substitution at position 36 selected from the group consisting of valine, leucine, isoleuceine and alanine.

150. The modified cell or the nucleic acid of any one of claims 126-144, wherein the FKBP12 variant polypeptide region is a FKBP12v36 polypeptide region.

151. The modified cell of any one of claims 145-147, wherein the ligand is rimiducid.

152. The modified cell of any one of claims 145-147, wherein the ligand is AP20187. 153. The modified cell of any one of claims 126-152, wherein the FRB variant polypeptide binds to a C7 rapalog.

154. The modified cell of any one of claims 126-153, wherein the FRB variant polypeptide comprises an amino acid substitution at position T2098 or W2101.

155. The modified cell of any one of claims 126-154, wherein the FRB variant polypeptide region is selected from the group consisting of KLW (T2098L)(FRBL), KTF (W2101F), and KLF (T2098L, W2101F).

156. The modified cell of any one of claims 126-155, wherein the FRB variant polypeptide region is FRBL.

157. The modified cell of any one of claims 126-156, wherein the FRB variant polypeptide region binds to a rapalog selected from the group consisting of S-o,p-dimethoxyphenyl (DMOP)-rapamycin, R-Isopropoxyrapamycin, C7-Isobutyloxyrapamycin, and S-Butanesulfonamidorap.

158. The modified cell or the nucleic acid of any one of claims 126-157, wherein the cell or the nucleic acid comprises a polynucleotide that encodes a chimeric antigen receptor, wherein the chimeric antigen receptor comprises (i) a transmembrane region, (ii) a T cell activation molecule, and (iii) an antigen recognition moiety.

159. The modified cell or the nucleic acid of claim 158, wherein the T cell activation molecule is selected from the group consisting of an ITAM-containing, Signal 1 conferring molecule, a Syk polypeptide, a ZAP70 polypeptide, a CD3ζ polypeptide, and an Fc epsilon receptor gamma (FcεR1γ) subunit polypeptide.

160. The modified cell or the nucleic acid of any one of claim 158 or 159, wherein the antigen recognition moiety is a single chain variable fragment.

161. The modified cell or the nucleic acid of any one of claims 158-160, wherein the transmembrane region is a CD8 transmembrane region.

162. The modified cell or the nucleic acid of any one of claims 158-161, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

163. The modified cell or the nucleic acid of any one of claims 158-162 wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

164. The modified cell or the nucleic acid of any one of claims 126-157, wherein the cell comprises a polynucleotide encoding a recombinant T cell receptor, wherein the recombinant T cell receptor binds to an antigenic polypeptide selected from the group consisting of PRAME, Bob-1, and NY-ESO-1.

165. The modified cell or the nucleic acid of any one of claims 126-164, wherein the pro-apoptotic polypeptide is selected from the group consisting of caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-xL, Bcl-2, RIPK3, and RIPK1-RHIM.

166. The modified cell or the nucleic acid of any one of claims 126-165, wherein the pro-apoptotic polypeptide is a caspase polypeptide.

167. The modified cell or the nucleic acid of claim 166, wherein the pro-apoptotic polypeptide is a Caspase-9 polypeptide.

168. The nucleic acid of cell of claim 167, wherein the Caspase-9 polypeptide lacks the CARD domain.

169. The modified cell or the nucleic acid of any one of claim 167 or 168, wherein the caspase polypeptide comprises the amino acid sequence of SEQ ID NO: 300.

170. The modified cell or the nucleic acid of any one of claims 166-168, wherein the caspase polypeptide is a modified Caspase-9 polypeptide comprising an amino acid substitution selected from the group consisting of the catalytically active caspase variants in Tables 5 or 6.

171. The modified cell or the nucleic acid of claim 170, wherein the caspase polypeptide is a modified Caspase-9 polypeptide comprising an amino acid sequence selected from the group consisting of D330A, D330E, and N405Q.

172. The modified cell or the nucleic acid of any one of claims 126-171, wherein the truncated MyD88 polypeptide has the amino acid sequence of SEQ ID NO: 214 or 305, or a functional fragment thereof.

173. The modified cell or the nucleic acid of any one of claims 126-171, wherein the MyD88 polypeptide has the amino acid sequence of SEQ ID NO: 282, or a functional fragment thereof.

174. The modified cell or the nucleic acid of any one of claims 126-173, wherein the cytoplasmic CD40 polypeptide has the amino acid sequence of SEQ ID NO: 216, or a functional fragment thereof.

175. The modified cell of claim 126, wherein,
a) the chimeric pro-apoptotic polypeptide comprises a Caspase-9 polypeptide lacking the CARD domain and a FKBP12v36 polypeptide region; and
b) the chimeric costimulating polypeptide comprises a truncated MyD88 polypeptide region lacking the TIR domain and a FRBL polypeptide region and a FKBP12 polypeptide region.

176. The modified cell of claim 126, wherein,
a) the chimeric pro-apoptotic polypeptide comprises a Caspase-9 polypeptide lacking the CARD domain and a FKBP12v36 polypeptide region; and
b) the chimeric costimulating polypeptide comprises a truncated MyD88 polypeptide region lacking the TIR domain, a CD40 cytoplasmic polypeptide region lacking the extracellular domain, a FRBL polypeptide region and a FKBP12 polypeptide region.

177. The nucleic acid of claim 142, wherein,
a) the chimeric pro-apoptotic polypeptide comprises a Caspase-9 polypeptide lacking the CARD domain and a FKBP12v36 polypeptide region; and
b) the chimeric costimulating polypeptide comprises a truncated MyD88 polypeptide region lacking the TIR domain and a FRBL polypeptide region and a FKBP12 polypeptide region.

178. The nucleic acid of claim 142, wherein,
a) the chimeric pro-apoptotic polypeptide comprises a Caspase-9 polypeptide lacking the CARD domain and a FKBP12v36 polypeptide region; and
b) the chimeric costimulating polypeptide comprises a truncated MyD88 polypeptide region lacking the TIR domain, a CD40 cytoplasmic polypeptide region lacking the extracellular domain, a FRBL polypeptide region and a FKBP12 polypeptide region.

179. The modified cell of any one of claim 126-131, 141, or 142-176, wherein the cell is a T cell, tumor infiltrating lymphocyte, NK-T cell, or NK cell.

180. The modified cell of any one of claim 126-131 or 141-176, wherein the cell is a T cell, NK-T cell, or NK cell.

181. The modified cell of any one of claim 126-131 or 141-176, wherein the cell is a T cell.

182. The modified cell of any one of claim 126-131 or 141-176, wherein the cell is a primary T cell.

183. The modified cell of any one of claim 126-131 or 141-176, wherein the cell is a cytotoxic T cell.

184. The modified cell of any one of claim 126-131 or 141-176, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

185. The modified cell of any one of claim 126-131 or 141-176, wherein the T cell is a helper T cell.

186. The modified cell of any one of claim 126-131 or 141-176, wherein the cell is obtained or prepared from bone marrow.

187. The modified cell of any one claim 126-131 or 141-176, wherein the cell is obtained or prepared from umbilical cord blood.

188. The modified cell of any one of claim 126-131 or 141-176, wherein the cell is obtained or prepared from peripheral blood.

189. The modified cell of any one of claim 126-131 or 141-176, wherein the cell is obtained or prepared from peripheral blood mononuclear cells.

190. The modified cell of any one of claim 126-131 or 141-176, wherein the cell is a human cell.

191. The modified cell of any one of claim 126-131, 141-176 or 179-190, wherein the modified cell is transduced or transfected in vivo.

192. The modified cell of any one of claim 126-131, 141-174 or 179-190, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

193. A kit or composition comprising nucleic acid comprising
a) a first polynucleotide encoding a chimeric pro-apoptotic polypeptide, wherein the chimeric pro-apoptotic polypeptide comprises
   i) a pro-apoptotic polypeptide region; and
   ii) a FKBP12 variant polypeptide region; and
b) a second polynucleotide encoding a chimeric costimulating polypeptide, wherein the chimeric costimulating polypeptide comprises
   i) a FRB polypeptide or FRB variant polypeptide region;
   ii) a FKBP12 polypeptide region; and
   iii) a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain.

194. The kit or composition of claim 193, wherein the chimeric costimulating polypeptide comprises a MyD88 polypeptide region or a truncated MyD88 polypeptide region lacking the TIR domain.

195. The kit or composition of any one of claims 193-194, wherein the chimeric costimulating polypeptide comprises a truncated MyD88 polypeptide region lacking the TIR domain and a CD40 cytoplasmic polypeptide region lacking the CD40 extracellular domain.

196. The kit or composition of any one of claims 193-195, further comprising a third polynucleotide, wherein the third polynucleotide encodes a heterologous protein.

197. The kit or composition of claim 196, wherein the heterologous protein is a chimeric antigen receptor.

198. The kit or composition of claim 196, wherein the heterologous protein is a recombinant TCR.

199. The kit or composition of claim 193, wherein the nucleic acid is a nucleic acid of any one of claim 132-139, 142-174, or 177-178.

200. The kit or composition of any one of claims 193-199, further comprising a third polynucleotide, wherein the third polynucleotide encodes a heterologous protein.

201. The kit or composition of 200, wherein the heterologous protein is a chimeric antigen receptor.

202. The kit or composition of claim 200, wherein the heterologous protein is a recombinant TCR.

203. The kit or composition of any one of claims 194-199, comprising a virus, wherein the virus comprises the first and the second polynucleotide.

204. The kit or composition of any one of claims 199-202, comprising a virus, wherein the virus comprises the first, second, and third polynucleotides.

205. The kit or composition of any one of claims 200-202, comprising a virus, wherein the virus comprises the first and third polynucleotides.

206. The kit or composition of any one of claims 200-202, comprising a virus, wherein the virus comprises the second and third polynucleotides.

207. The kit or composition of any one of claims 200-202, comprising a virus, wherein the virus comprises the first, second, and third polynucleotides.

208. A method for expressing a chimeric pro-apoptotic polypeptide and a chimeric costimulating polypeptide, wherein
a) the chimeric pro-apoptotic polypeptide comprises
   i) a pro-apoptotic polypeptide region; and
   ii) a FKBP12 variant polypeptide region; and
b) the chimeric costimulating polypeptide comprises
c) a FRB or FRB variant polypeptide region;
d) a FKBP12 polypeptide region; and
e) a MyD88 polypeptide region or a truncated MyD88 polypeptide region lacking the TIR domain, or a MyD88 polypeptide region or a truncated MyD88 polypeptide region lacking the TIR domain and a CD40 cytoplasmic polypeptide region lacking the CD40 extracellular domain.
   comprising contacting a nucleic acid of any one of claim 132-139, 142-174, or 177-178 with a cell under conditions in which the nucleic acid is incorporated into the cell, whereby the cell expresses the chimeric pro-apoptotic polypeptide and the chimeric costimulating polypeptide from the incorporated nucleic acid.

209. The method of claim 208, wherein the nucleic acid is contacted with the cell ex vivo.

210. The method of claim 208, wherein the nucleic acid is contacted with the cell in vivo.

211. A method of stimulating an immune response in a subject, comprising:
a) transplanting modified cells of any one of claim 126-131, 141-176, or 179-192 into the subject, and
b) after (a), administering an effective amount of a rapamycin or a rapalog that binds to the FRB polypeptide or FRB variant polypeptide region of the chimeric stimulating polypeptide to stimulate a cell mediated immune response.

212. A method of administering a ligand to a subject who has undergone cell therapy using modified cells, comprising administering rapamycin or a rapalog to the subject, wherein the modified cells comprise modified cells of any one of claim 126-131, 141-176, or 179-192.

213. A method of controlling activity of transplanted modified cells in a subject, comprising:
a) transplanting a modified cell of any one of claim 126-131, 141-176, or 179-192; and
b) after (a), administering an effective amount of rapamycin or a rapalog that binds to the FRB or FRB variant polypeptide region of the chimeric stimulating polypeptide to stimulate the activity of the transplanted modified cells.

214. A method for treating a subject having a disease or condition associated with an elevated expression of a target antigen expressed by a target cell, comprising a) transplanting an effective amount of modified cells into the subject;
wherein the modified cells comprise a modified cell of any one of claim 126-131, 141-176, or 179-192, wherein the modified cell comprises a chimeric antigen receptor comprising an antigen recognition moiety that binds to the target antigen, and
b) after a), administering an effective amount of rapamycin or a rapalog that binds to the FRB polypeptide or FRB variant region of the chimeric stimulating polypeptide to reduce the number or concentration of target antigen or target cells in the subject.

215. The method of claim 214, wherein the target antigen is a tumor antigen.

216. A method for treating a subject having a disease or condition associated with an elevated expression of a target antigen expressed by a target cell, comprising
a) administering to the subject an effective amount of modified cells, wherein the modified cells comprise a modified cell of any one of claim 126-131, 141-176, or 179-192, wherein the modified cell comprises a recombinant T cell receptor that recognizes and binds to the target antigen, and
b) after a), administering an effective amount of rapamycin or a rapalog that binds to the FRB or FRB variant polypeptide region of the chimeric stimulating polypeptide to reduce the number or concentration of target antigen or target cells in the subject.

217. A method for reducing the size of a tumor in a subject, comprising
a) administering a modified cell of any one of claim 126-131, 141-176, or 179-192 to the subject, wherein the cell comprises a chimeric antigen receptor comprising an antigen recognition moiety that binds to an antigen on the tumor; and
b) after a), administering an effective amount of rapamycin or a rapalog that binds to the FRB or FRB variant polypeptide region of the chimeric stimulating polypeptide to reduce the size of the tumor in the subject.

218. The method of any one of claims 214-217, comprising measuring the number or concentration of target cells in a first sample obtained from the subject before administering second ligand, measuring the number or concentration of target cells in a second sample obtained from the subject after administering the ligand, and determining an increase or decrease of the number or concentration of target cells in the second sample compared to the number or concentration of target cells in the first sample.

219. The method of claim 218, wherein the concentration of target cells in the second sample is decreased compared to the concentration of target cells in the first sample.

220. The method of claim 218, wherein the concentration of target cells in the second sample is increased compared to the concentration of target cells in the first sample.

221. The method of any one of claims 211-220, wherein the subject has received a stem cell transplant before or at the same time as administration of the modified cells.

222. The method of any one of claims 211-221, wherein at least 1×106 transduced or transfected modified cells are administered to the subject.

223. The method of any one of claims 211-221, wherein at least 1×107 transduced or transfected modified cells are administered to the subject.

224. The method of any one of claims 211-221, wherein at least 1×108 modified cells are administered to the subject.

225. The method of any one of claims 211-224, wherein the FKBP12 variant polypeptide region is FKBP12v36 and the ligand that binds to the FKBP12 variant polypeptide region is AP1903.

226. A method of controlling survival of transplanted modified cells in a subject, comprising
a) transplanting modified cells of any one of claim 126-131, 141-176, or 179-192 into the subject, and
b) after (a), administering to the subject a ligand that binds to the FKBP12 variant polypeptide region of the chimeric pro-apoptotic polypeptide in an amount effective to kill less than 95% of the modified cells that express the chimeric pro-apoptotic polypeptide.

227. The method of any one of claims 211-225, further comprising after (b), administering to the subject a ligand that binds to the FKBP12 variant polypeptide region of the chimeric pro-apoptotic polypeptide in an amount effective to kill less than 95% of the modified cells that express the chimeric pro-apoptotic polypeptide.

228. The method of any one of claim 226 or 227, wherein a ligand that binds to the FKBP12 variant polypeptide region is administered in an amount effective to kill less than 40% of the modified cells that express the chimeric pro-apoptotic polypeptide.

229. The method of any one of claim 226 or 227, wherein a ligand that binds to the FKBP12 variant polypeptide region is administered in an amount effective to kill less than 50% of the modified cells that express the chimeric pro-apoptotic polypeptide.

230. The method of any one of claim 226 or 227, wherein the a ligand that binds to the FKBP12 variant polypeptide region is administered in an amount effective to kill less than 60% of the modified cells that express the chimeric pro-apoptotic polypeptide.

231. The method of any one of claim 226 or 227, wherein the a ligand that binds to the FKBP12 variant polypeptide region is administered in an amount effective to kill less than 70% of the modified cells that express the chimeric pro-apoptotic polypeptide.

232. The method of any one of claim 226 or 227, wherein the a ligand that binds to the FKBP12 variant polypeptide region is administered in an amount effective to kill less than 90% of the modified cells that express the chimeric pro-apoptotic polypeptide.

233. The method of any one of claim 226 or 227, wherein the a ligand that binds to the FKBP12 variant polypeptide region is administered in an amount effective to kill at least 90% of the modified cells that express the chimeric pro-apoptotic polypeptide.

234. The method of any one of claim 226 or 227, wherein the a ligand that binds to the FKBP12 variant polypeptide region is administered in an amount effective to kill at least 95% of the modified cells that express the chimeric pro-apoptotic polypeptide.

235. The method of any one of claims 226-227, wherein the chimeric costimulating polypeptide comprises a FRBL region.

236. The method of any one of claims 221-225, wherein more than one dose of the ligand is administered to the subject.

237. The method of any one of claims 226-236, wherein more than one dose of the ligand that binds to the FKBP12 variant polypeptide region is administered to the subject.

238. The method of any one of claims 211-236, further comprising identifying a presence or absence of a condition in the subject that requires the removal of the modified cells from the subject; and administering a ligand that binds to the FKBP12 variant polypeptide region, maintaining a subsequent dosage of the ligand, or adjusting a subsequent dosage of the ligand to the subject based on the presence or absence of the condition identified in the subject.

239. The method of any one of claims 211-236, further comprising receiving information comprising presence or absence of a condition in the subject that requires the removal of the modified cells from the subject; and administering the a ligand that binds to the FKBP12 variant polypeptide region, maintaining a subsequent dosage of the ligand, or adjusting a subsequent dosage of the ligand to the subject based on the presence or absence of the condition identified in the subject.

240. The method of any one of claims 211-236, further comprising identifying a presence or absence of a condition in the subject that requires the removal of the modified cells from the subject; and transmitting the presence, absence or stage of the condition identified in the subject to a decision maker who administers a ligand that binds to the FKBP12 variant polypeptide region, maintains a subsequent dosage of the ligand, or adjusts a subsequent dosage of the ligand administered to the subject based on the presence, absence or stage of the condition identified in the subject.

241. The method of any one of claims 211-236, further comprising identifying a presence or absence of a condition in the subject that requires the removal of the modified cells from the subject; and transmitting an indication to administer the a ligand that binds to the FKBP12 variant polypeptide region, maintain a subsequent dosage of the ligand, or adjust a subsequent dosage of the ligand administered to the subject based on the presence, absence or stage of the condition identified in the subject.

242. The method of any one of claims 211-241, wherein the subject has cancer.

243. The method of any one of claims 211-241, wherein the modified cell is delivered to a tumor bed.

244. The method of any one of claim 242 or 243, wherein the cancer is present in the blood or bone marrow of the subject.

245. The method of any one of claims 211-241, wherein the subject has a blood or bone marrow disease.

246. The method of any one of claims 211-241, wherein the subject has been diagnosed with sickle cell anemia or metachromatic leukodystrophy.

247. The method of any one of claims 211-241, wherein the patient has been diagnosed with a condition selected from the group consisting of a primary immune deficiency condition, hemophagocytosis lymphohistiocytosis (HLH) or other hemophagocytic condition, an inherited marrow failure condition, a hemoglobinopathy, a metabolic condition, and an osteoclast condition.

248. The method of any one of claims 211-241, wherein the patient has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XLP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

249. A nucleic acid comprising a promoter operably linked to a polynucleotide coding for a chimeric pro-apoptotic polypeptide, wherein the chimeric pro-apoptotic polypeptide comprises
a) a pro-apoptotic polypeptide region;
b) a FKBP12-Rapamycin binding domain (FRB) polypeptide or FRB variant polypeptide region; and
c) a FKBP12 variant polypeptide region.

250. The nucleic acid of claim 249, wherein the order of regions (a), (b), and (c), from the amino terminus to the carboxyl terminus of the chimeric pro-apoptotic polypeptide is (c), (b), (a).

251. The nucleic acid of claim 249, wherein the order of regions (a), (b), and (c), from the amino terminus to the carboxyl terminus of the chimeric pro-apoptotic polypeptide is (b), (c), (a).

252. The nucleic acid of any one of claim 250 or 251, wherein (b) and (c) are amino terminal to the pro-apoptotic polypeptide.

253. The nucleic acid of any one of claim 250 or 251, wherein (b) and (c) are carboxyl terminal to the pro-apoptotic polypeptide.

254. The nucleic acid of any one of claims 259 to 253, wherein the chimeric pro-apoptotic polypeptide further comprises linker polypeptides between regions (a), (b), and (c).

255. The nucleic acid of any one of claims 249-254, wherein the FKBP12 variant polypeptide region binds to a ligand with at least 100 times more affinity than the ligand binds to a wild type FKBP12 polypeptide region.

256. The nucleic acid of any one of claims 249-254, wherein the FKBP12 variant polypeptide region binds to a ligand with at least 500 times more affinity than the ligand binds to the a wild type FKBP12 polypeptide region.

257. The nucleic acid of any one of claims 249-254, wherein the FKBP12 variant polypeptide region binds to a ligand with at least 1000 times more affinity than the ligand binds to a wild type FKBP12 polypeptide region.

258. The nucleic acid of any one of claims 249-257, wherein the FKBP12 variant comprises an amino acid substitution at amino acid residue 36.

259. The nucleic acid of claim 258, wherein the amino acid substitution at position 36 selected from the group consisting of valine, leucine, isoleuceine and alanine.

260. The nucleic acid of any one of claims 249-259, wherein the FKBP12 variant polypeptide region is a FKBP12v36 polypeptide region.

261. The nucleic acid of any one of claims 255-260, wherein the ligand is rimiducid.

262. The nucleic acid of any one of claims 255-260, wherein the ligand is AP20187 or N1510.

263. The nucleic acid of any one of claims 249-262, wherein the FRB variant polypeptide binds to a C7 rapalog.

264. The nucleic acid of any one of claims 249-263, wherein the FRB variant polypeptide comprises an amino acid substitution at position T2098 or W2101.

265. The nucleic acid of any one of claims 249-264, wherein the FRB variant polypeptide region is selected from the group consisting of KLW (T2098L) (FRBL), KTF (W2101F), and KLF (T2098L, W2101F).

266. The nucleic acid of any one of claims 249-265, wherein the FRB variant polypeptide region is FRBL.

267. The nucleic acid of any one of claims 249-266, wherein the FRB variant polypeptide region binds to a rapalog selected from the group consisting of S-o,p-dimethoxyphenyl (DMOP)-rapamycin, R-Isopropoxyrapamycin, C7-Isobutyloxyrapamycin, and S-Butanesulfonamidorap.

268. The nucleic acid of any one of claims 249-267, wherein the promoter is operably linked to a second polynucleotide, wherein the second polynucleotide encodes a heterologous protein.

269. The nucleic acid of claim 268, wherein the heterologous protein is a chimeric antigen receptor.

270. The nucleic acid of claim 268, wherein the heterologous protein is a recombinant TCR.

271. The nucleic acid of any one of claims 249-268, wherein the nucleic acid further comprises a polynucleotide encoding a linker polypeptide between the polynucleotide that encodes the chimeric pro-apoptotic polypeptide and the second polynucleotide, wherein the linker polypeptide separates the translation products of the first and second polynucleotides during or after translation.

272. The nucleic acid of claim 271, wherein the linker polypeptide is a 2A polypeptide.

273. The nucleic acid of any one of claim 269, or 271-272, wherein the chimeric antigen receptor comprises (i) a transmembrane region, (ii) a T cell activation molecule, and (iii) an antigen recognition moiety.

274. The nucleic acid of claim 273, wherein the T cell activation molecule is selected from the group consisting of an ITAM-containing, Signal 1 conferring molecule, a Syk polypeptide, a ZAP70 polypeptide, a CD3ζ polypeptide, and an Fc epsilon receptor gamma (FcεR1γ) subunit polypeptide.

275. The nucleic acid of claim 273, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.N23.

276. The nucleic acid of any one of claims 273-275, wherein the antigen recognition moiety is a single chain variable fragment.

277. The nucleic acid of any one of claims 273-276, wherein the transmembrane region is a CD8 transmembrane region.

278. The nucleic acid of any one of claims 273-277, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

279. The nucleic acid of any one of claims 273-277 wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

280. The nucleic acid of any one of claims 270-272, wherein the recombinant T cell receptor binds to an antigenic polypeptide selected from the group consisting of PRAME, Bob-1, and NY-ESO-1.

281. The nucleic acid of any one of claims 249-280, further comprising a polynucleotide encoding a chimeric costimulatory polypeptide comprising a MyD88 polypeptide region or a truncated MyD88 polypeptide region lacking the TIR domain.

282. The nucleic acid of claim 281, wherein the chimeric costimulatory polypeptide further comprises a CD40 cytoplasmic polypeptide lacking the CD40 extracellular domain.

283. The nucleic acid of any one of claims 281-282, wherein the chimeric costimulatory polypeptide further comprises a membrane targeting region.

284. The nucleic acid of claim 283, wherein the membrane targeting region comprises a myristoylation region.

285. The nucleic acid of any one of claims 282-284, wherein the truncated MyD88 polypeptide has the amino acid sequence of SEQ ID NO: 214 or 969, or a functional fragment thereof.

286. The nucleic acid of any one of claims 282-284, wherein the MyD88 polypeptide has the amino acid sequence of SEQ ID NO: 282, or a functional fragment thereof.

287. The nucleic acid of any one of claims 282-286, wherein the cytoplasmic CD40 polypeptide has the amino acid sequence of SEQ ID NO: 216, or a functional fragment thereof.

288. The nucleic acid of any one of claims 249-287, wherein the pro-apoptotic polypeptide is selected from the group consisting of Caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-xL, Bcl-2, RIPK3, and RIPK1-RHIM.

289. The nucleic acid of any one of claims 249-288, wherein the pro-apoptotic polypeptide is a caspase polypeptide.

290. The nucleic acid of claim 289, wherein the pro-apoptotic polypeptide is a Caspase-9 polypeptide.

291. The nucleic acid of cell of claim 290, wherein the Caspase-9 polypeptide lacks the CARD domain.

292. The nucleic acid of any one of claim 289 or 290, wherein the caspase polypeptide comprises the amino acid sequence of SEQ ID NO: 300.

293. The nucleic acid of any one of claims 289-290, wherein the caspase polypeptide is a modified Caspase-9 polypeptide comprising an amino acid substitution selected from the group consisting of the catalytically active caspase variants in Tables 5 or 6.

294. The nucleic acid of claim 294, wherein the caspase polypeptide is a modified Caspase-9 polypeptide comprising an amino acid sequence selected from the group consisting of D330A, D330E, and N405Q.

295. The nucleic acid of any one of claims 249-294, wherein, the chimeric pro-apoptotic polypeptide comprises a Caspase-9 polypeptide lacking the CARD domain, a FKBP12v36 polypeptide region; and a FRBL polypeptide region.

296. A chimeric pro-apoptotic polypeptide encoded by a nucleic acid of any one of claims 249-295.

297. A modified cell transfected or transduced with a nucleic acid of any one of claims 249-295.

298. The modified cell of claim 297, wherein the modified cell comprises a polynucleotide that encodes a chimeric antigen receptor.

299. The modified cell of claim 297, wherein the modified cell comprises a polynucleotide that encodes a recombinant TCR.

300. The modified cell of claim 298, wherein the chimeric antigen receptor comprises (i) a transmembrane region, (ii) a T cell activation molecule, and (iii) an antigen recognition moiety.

301. The modified cell of claim 300, wherein the T cell activation molecule is selected from the group consisting of an ITAM-containing, Signal 1 conferring molecule, a Syk polypeptide, a ZAP70 polypeptide, a CD3ζ polypeptide, and an Fc epsilon receptor gamma (FcεR1γ) subunit polypeptide.

302. The modified cell of claim 300, wherein the T cell activation molecule is selected from the group consisting of an ITAM-containing, Signal 1 conferring molecule, a CD3ζ polypeptide, and an Fc epsilon receptor gamma (FcεR1γ) subunit polypeptide.P6.

303. The modified cell of any one of claims 300-302, wherein the antigen recognition moiety is a single chain variable fragment.

304. The modified cell of any one of claims 300-303, wherein the transmembrane region is a CD8 transmembrane region.

305. The modified cell of any one of claims 300-304, wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

306. The modified cell of any one of claims 300-304 wherein the antigen recognition moiety binds to an antigen selected from the group consisting of an antigen on a tumor cell, an antigen on a cell involved in a hyperproliferative disease, a viral antigen, a bacterial antigen, CD19, PSCA, Her2/Neu, PSMA, Muc1Muc1, Muc1, ROR1, Mesothelin, GD2, CD123, Muc16, CD33, CD38, and CD44v6.

307. The modified cell of claim 299, wherein the recombinant T cell receptor binds to an antigenic polypeptide selected from the group consisting of PRAME, Bob-1, and NY-ESO-1.

308. The modified cell of claim 297, wherein the modified cell comprises a polynucleotide that encodes a MyD88 polypeptide or a truncated MyD88 polypeptide region lacking the TIR domain.

309. The modified cell of claim 308, wherein the modified cell comprises a polynucleotide that encodes a chimeric costimulating polypeptide, wherein the chimeric costimulating polypeptide encodes a MyD88 polypeptide or a truncated MyD88 polypeptide region lacking the TIR domain and a CD40 cytoplasmic polypeptide lacking the CD40 extracellular domain.

310. The modified cell of any one of claims 308-309, wherein the truncated MyD88 polypeptide has the amino acid sequence of SEQ ID NO: 214 or 969, or a functional fragment thereof.

311. The modified cell of any one of claims 308-310, wherein the MyD88 polypeptide has the amino acid sequence of SEQ ID NO: 282, or a functional fragment thereof.

312. The modified cell of any one of claims 309-311, wherein the cytoplasmic CD40 polypeptide has the amino acid sequence of SEQ ID NO: 216, or a functional fragment thereof.

313. The modified cell of any one of claims 297-312, wherein the cell is a T cell, tumor infiltrating lymphocyte, NK-T cell, or NK cell.

314. The modified cell of any one of claims 297-312, wherein the cell is a T cell, NK-T cell, or NK cell.

315. The modified cell of any one of claims 297-312, wherein the cell is a T cell.

316. The modified cell of any one of claims 297-312, wherein the cell is a primary T cell.

317. The modified cell of any one of claims 297-312, wherein the cell is a cytotoxic T cell.

318. The modified cell of any one of claims 297-312, wherein the cell is selected from the group consisting of embryonic stem cell (ESC), inducible pluripotent stem cell (iPSC), non-lymphocytic hematopoietic cell, non-hematopoietic cell, macrophage, keratinocyte, fibroblast, melanoma cell, tumor infiltrating lymphocyte, natural killer cell, natural killer T cell, or T cell.

319. The modified cell of any one of claims 297-312, wherein the T cell is a helper T cell.

320. The modified cell of any one of claims 297-312, wherein the cell is obtained or prepared from bone marrow.

321. The modified cell of any one claims 297-312, wherein the cell is obtained or prepared from umbilical cord blood.

322. The modified cell of any one of claims 297-312, wherein the cell is obtained or prepared from peripheral blood.

323. The modified cell of any one of claims 297-312, wherein the cell is obtained or prepared from peripheral blood mononuclear cells.

324. The modified cell of any one of claims 297-323, wherein the cell is a human cell.

325. The modified cell of any one of claims 297-324, wherein the modified cell is transduced or transfected in vivo.

326. The modified cell of any one of claims 297-325, wherein the cell is transfected or transduced by the nucleic acid vector using a method selected from the group consisting of electroporation, sonoporation, biolistics (e.g., Gene Gun with Au-particles), lipid transfection, polymer transfection, nanoparticles, or polyplexes.

327. A kit or composition comprising nucleic acid comprising a polynucleotide coding for a chimeric pro-apoptotic polypeptide, wherein the chimeric pro-apoptotic polypeptide comprises
a) a pro-apoptotic polypeptide region;
b) a FKBP12-Rapamycin binding domain (FRB) polypeptide or FRB variant polypeptide region; and
c) a FKBP12 variant polypeptide region.

328. The kit or composition of claim 327, wherein the FKBP12 variant comprises an amino acid substitution at amino acid residue 36.

329. The kit or composition of claim 328, wherein the amino acid substitution at position 36 selected from the group consisting of valine, leucine, isoleuceine and alanine.

330. The kit or composition of any one of claims 327-329, wherein the FKBP12 variant polypeptide region is a FKBP12v36 polypeptide region.

331. The kit or composition of any one of claims 327-330, wherein the FKBP12 variant polypeptide region binds to rimiducid.

332. The kit or composition of any one of claims 327-331, wherein the FKBP12 variant polypeptide region binds to AP20187 of AP1510.

333. The kit or composition of any one of claims 327-332, wherein the FRB variant polypeptide binds to a C7 rapalog.

334. The kit or composition of any one of claims 327-333, wherein the FRB variant polypeptide comprises an amino acid substitution at position T2098 or W2101.

335. The kit or composition of any one of claims 327-334, wherein the FRB variant polypeptide region is selected from the group consisting of KLW (T2098L) (FRBL), KTF (W2101F), and KLF (T2098L, W2101F).

336. The kit or composition of any one of claims 327-335, wherein the FRB variant polypeptide region is FRBL.

337. The kit or composition of any one of claims 327-336, wherein the FRB variant polypeptide region binds to a rapalog selected from the group consisting of S-o,p-dimethoxyphenyl (DMOP)-rapamycin, R-Isopropoxyrapamycin, C7-Isobutyloxyrapamycin, and S-Butanesulfonamidorap.

338. The kit or composition of any one of claims 327-337, wherein the nucleic acid is a nucleic acid of any one of claims 249-N41.

339. A method for expressing a chimeric pro-apoptotic polypeptide, wherein the chimeric pro-apoptotic polypeptide comprises
a) a pro-apoptotic polypeptide region;
b) a FRB or FRB variant polypeptide region; and
c) a FKBP12 variant polypeptide region.
   comprising contacting a nucleic acid of any one of claims 249-295 with a cell under conditions in which the nucleic acid is incorporated into the cell, whereby the cell expresses the chimeric pro-apoptotic polypeptide and the chimeric costimulating polypeptide from the incorporated nucleic acid.

340. The method of claim 339, wherein the nucleic acid is contacted with the cell ex vivo.

341. The method of claim 339, wherein the nucleic acid is contacted with the cell in vivo.

342. A method of controlling survival of transplanted modified cells in a subject, comprising:
a) transplanting modified cells of any one of claims 297 to 326 into the subject; and
b) after (a), administering to the subject
   i) a first ligand that binds to the FRB or FRB variant polypeptide region of the chimeric pro-apoptotic polypeptide; or
   ii) a second ligand that binds to the FKBP12 variant polypeptide region of the chimeric pro-apoptotic polypeptide wherein the first ligand or the second ligand are administered in an amount effective to kill at least 30% of the modified cells that express the chimeric pro-apoptotic polypeptide.

343. The method of claim 342, wherein the first ligand or the second ligand are administered in an amount effective to kill at least 40% of the modified cells that express the chimeric pro-apoptotic polypeptide.

344. The method of claim 342, wherein the first ligand or the second ligand are administered in an amount effective to kill at least 50% of the modified cells that express the chimeric pro-apoptotic polypeptide.

345. The method of claim 342, wherein the first ligand or the second ligand are administered in an amount effective to kill at least 60% of the modified cells that express the chimeric pro-apoptotic polypeptide.

346. The method of claim 342, wherein the first ligand or the second ligand are administered in an amount effective to kill at least 70% of the modified cells that express the chimeric pro-apoptotic polypeptide.

347. The method of claim 342, wherein the first ligand or the second ligand are administered in an amount effective to kill at least 80% of the modified cells that express the chimeric pro-apoptotic polypeptide.

348. The method of claim 342, wherein the first ligand or the second ligand are administered in an amount effective to kill at least 90% of the modified cells that express the chimeric pro-apoptotic polypeptide.

349. The method of claim 342, wherein the first ligand or the second ligand are administered in an amount effective to kill at least 95% of the modified cells that express the chimeric pro-apoptotic polypeptide.

350. The method of claim 342, wherein the first ligand or the second ligand are administered in an amount effective to kill at least 99% of the modified cells that express the chimeric pro-apoptotic polypeptide.

351. A method of administering a first ligand or a second ligand to a subject who has undergone cell therapy using modified cells that express a chimeric pro-apoptotic polypeptide, wherein the modified cells comprise a nucleic acid of any one of claims 249-N45, wherein the first ligand or the second ligand is administered in an amount effective to kill at least 30% of the modified cells that express the chimeric pro-apoptotic polypeptide.

352. The method of claim 351, wherein the first ligand binds to the FRB or FRB variant polypeptide region and the second ligand binds to the FKBP12 variant polypeptide region of the chimeric pro-apoptotic polypeptide.

353. The method of any one of claims 351-352, wherein the first ligand or the second ligand are administered in an amount effective to kill less at least 40% of the modified cells that express the chimeric pro-apoptotic polypeptide.

354. The method of any one of claims 351-352, wherein the first ligand or the second ligand are administered in an amount effective to kill less at least 50% of the modified cells that express the chimeric pro-apoptotic polypeptide.

355. The method of any one of claims 351-352, wherein the first ligand or the second ligand are administered in an amount effective to kill less at least 60% of the modified cells that express the chimeric pro-apoptotic polypeptide.

356. The method of any one of claims 351-352, wherein the first ligand or the second ligand are administered in an amount effective to kill less at least 70% of the modified cells that express the chimeric pro-apoptotic polypeptide.

357. The method of any one of claims 351-352, wherein the first ligand or the second ligand are administered in an amount effective to kill less at least 80% of the modified cells that express the chimeric pro-apoptotic polypeptide.

358. The method of any one of claims 351-352, wherein the first ligand or the second ligand are administered in an amount effective to kill at least 90% of the modified cells that express the chimeric pro-apoptotic polypeptide.

359. The method of any one of claims 351-352, wherein the first ligand or the second ligand are administered in an amount effective to kill at least 95% of the modified cells that express the chimeric pro-apoptotic polypeptide.

360. The method of any one of claims 351-352, wherein the first ligand or the second ligand are administered in an amount effective to kill at least 99% of the modified cells that express the chimeric pro-apoptotic polypeptide.

361. The method of any one of claims 342-360, wherein more than one dose of the ligand is administered to the subject.

362. The method of any one of claims 342-361, wherein the first ligand is rapamycin or a rapalog.

363. The method of claim 362, wherein the first ligand is a rapalog selected from the group consisting of S-o,p-dimethoxyphenyl (DMOP)-rapamycin, R-Isopropoxyrapamycin, C7-Isobutyloxyrapamycin, and S-Butanesulfonamidorap.

364. The method of any one of claims 342-360, wherein the second ligand is rimiducid, AP20187, or AP1510.

365. The method of claim 364, wherein the second ligand is rimiducid.

366. The method of any one of claims 342-365, wherein more than one dose of the first ligand or the second ligand is administered.

367. The method of any one of claims 342-366, wherein both the first ligand and the second ligand are administered.

368. The method of any one of claims 342-367, further comprising identifying a presence or absence of a condition in the subject that requires the removal of modified cells from the subject; and administering the first or the second ligand, or maintaining a subsequent dosage of the first or the second ligand, or adjusting a subsequent dosage of the first or second ligand to the subject based on the presence or absence of the condition identified in the subject.

369. The method of any one of claims 342-367, further comprising identifying a presence or absence of a condition in the subject that requires the removal of transfected or transduced therapeutic cells from the subject; and determining whether the first or the second ligand should be administered to the subject, or the dosage of the first or the second ligand subsequently administered to the subject is adjusted based on the presence or absence of the condition identified in the subject.

370. The method of any one of claims 342-369, further comprising receiving information comprising presence or absence of a condition in the subject that requires the removal of transfected or transduced modified cells from the subject; and administering the first ligand or the second ligand, maintaining a subsequent dosage of the first ligand or the second ligand, or adjusting a subsequent dosage of the first ligand or the second ligand to the subject based on the presence or absence of the condition identified in the subject.

371. The method of any one of claims 342-369, further comprising identifying a presence or absence of a condition in the subject that requires the removal of transfected or transduced modified cells from the subject; and transmitting the presence, absence or stage of the condition identified in the subject to a decision maker who administers the first ligand or the second ligand, maintains a subsequent dosage of the first ligand or the second ligand, or adjusts a subsequent dosage of the first ligand or the second ligand administered to the subject based on the presence, absence or stage of the condition identified in the subject.

372. The method of any one of claims 342-39, further comprising identifying a presence or absence of a condition in the subject that requires the removal of transfected or transduced modified cells from the subject; and transmitting an indication to administer the first ligand or the second ligand, maintain a subsequent dosage of the first ligand or the second ligand, or adjusts a subsequent dosage of the first ligand or the second ligand administered to the subject based on the presence, absence or stage of the condition identified in the subject.

373. The method of any one of claims 342-369, wherein alloreactive modified cells are present in the subject and the number of alloreactive modified cells is reduced by at least 90% after administration of the first ligand or the second ligand.

374. The method of any one of claims 342-369, wherein at least 1×106 transduced or transfected modified cells are administered to the subject.

375. The method of any one of claims 342-373, wherein at least 1×107 transduced or transfected modified cells are administered to the subject.

376. The method of any one of claims 342-373, wherein at least 1×108 transduced or transfected modified cells are administered to the subject.

377. The method of any one of claims 342-373, further comprising identifying the presence, absence or stage of graft versus host disease in the subject, and administering the first ligand or the second ligand, maintaining a subsequent dosage of the first ligand or the second ligand, or adjusting a subsequent dosage of the first ligand or the second ligand to the subject based on the presence, absence or stage of the graft versus host disease identified in the subject.

378. A method of administering a ligand to a subject who has undergone cell therapy using modified cells comprising administering the ligand to the subject, wherein the modified cells comprise a modified cell of any one of claims 297-326, wherein the ligand binds to a FKBP12 variant polypeptide region.

379. A method of administering rapamycin or a rapalog to a subject who has undergone cell therapy using modified cells comprising administering rapamycin or a rapalog to the subject, wherein the modified cells comprise a modified cell of any one of claims 297-326, wherein the rapamycin or rapalog binds to a FRB polypeptide or FRB variant polypeptide region.

380. The method of claim 378, wherein the ligand is selected from the group consisting of rapamycin, AP20187, and AP1510.

381. The method of any one of claims 342-179, wherein at least 30% of cells expressing the chimeric pro-apoptotic polypeptide are killed within 24 hours of administering the first ligand or the second ligand.

382. The method of claim 381, wherein at least 40% of cells expressing the chimeric pro-apoptotic polypeptide are killed within 24 hours of administering the first ligand or the second ligand.

383. The method of claim 381, wherein at least 50% of cells expressing the chimeric pro-apoptotic polypeptide are killed within 24 hours of administering the first ligand or the second ligand.

384. The method of claim 381, wherein at least 60% of cells expressing the chimeric pro-apoptotic polypeptide are killed within 24 hours of administering the first ligand or the second ligand.

385. The method of claim 381, wherein at least 70% of cells expressing the chimeric pro-apoptotic polypeptide are killed within 24 hours of administering the first ligand or the second ligand.

386. The method of claim 381, wherein at least 80% of cells expressing the chimeric pro-apoptotic polypeptide are killed within 24 hours of administering the first ligand or the second ligand.

387. The method of claim 381, wherein at least 90% of cells expressing the chimeric pro-apoptotic polypeptide are killed within 24 hours of administering the first ligand or the second ligand.

388. The method of claim 381, wherein at least 95% of cells expressing the chimeric pro-apoptotic polypeptide are killed within 24 hours of administering the first ligand or the second ligand.

389. The method of any one of claims 381-388, wherein at least 30%, at last 40%, at least 50%, at least 60%, at least 70%, at least 80%, at least 90% or at least 95% of cells expressing the chimeric pro-apoptotic polypeptide are killed within 90 minutes of administering the first ligand or the second ligand.

390. The method of any one of claims 342-389, wherein
a) the first ligand is administered to the subject, followed by the second ligand, or
b) the second ligand is administered to the subject, followed by the first ligand.

391. The method of any one of claims 342-390, wherein the subject is human.

392. The method of any one of claims 342-391, wherein the subject is selected from the group consisting of non-human primate, mouse, pig, cow, goat, rabbit, rat, guinea pig, hamster, horse, monkey, sheep, bird, and fish.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A modified cell, comprising
a) a first polynucleotide encoding a chimeric pro-apoptotic polypeptide, wherein the chimeric pro-apoptotic polypeptide comprises
(i) a pro-apoptotic polypeptide region;
(ii) a FKBP12-Rapamycin-Binding (FRB) domain polypeptide, or FRB variant polypeptide region; and
(iii) a FKBP12 or FKBP12 variant polypeptide region (FKBP12v); and
b) a second polynucleotide encoding a chimeric costimulating polypeptide, wherein the chimeric costimulating polypeptide comprises two FKBP12 variant polypeptide regions and
i) a MyD88 polypeptide region or a truncated MyD88 polypeptide region lacking the TIR domain; or
ii) a MyD88 polypeptide region or a truncated MyD88 polypeptide region lacking the TIR domain, and a CD40 cytoplasmic polypeptide region lacking the CD40 extracellular domain.

2. The modified cell of claim 1, wherein the chimeric costimulating polypeptide comprises two FKBP12 variant polypeptide regions, a truncated MyD88 polypeptide region lacking the TIR domain, and a CD40 cytoplasmic polypeptide region lacking the CD40 extracellular domain.

3. The modified cell of claim 1, wherein the cell further comprises a third polynucleotide encoding a chimeric antigen receptor or a recombinant T cell receptor.

4.-7. (canceled)

8. The modified cell of claim 1, wherein the cell is a T cell, tumor infiltrating lymphocyte, NK-T cell, or NK cell.

9.-15. (canceled)

16. A modified cell comprising
a) a first polynucleotide encoding a chimeric pro-apoptotic polypeptide, wherein the chimeric pro-apoptotic polypeptide comprises
i) a pro-apoptotic polypeptide region; and
ii) a FKBP12 variant polypeptide region; and
b) a second polynucleotide encoding a chimeric costimulating polypeptide, wherein the chimeric costimulating polypeptide comprises
i) a FKBP12-Rapamycin Binding (FRB) domain polypeptide or FRB variant polypeptide region;
ii) a FKBP12 polypeptide or FKBP12 variant polypeptide region; and
iii) a MyD88 polypeptide region or a truncated MyD88 polypeptide region lacking the TIR domain, or a MyD88 polypeptide region, or a truncated MyD88 polypeptide region lacking the TIR domain and a CD40 cytoplasmic polypeptide region lacking the CD40 extracellular domain.

17. The modified cell of claim 16, wherein the chimeric costimulating polypeptide comprises a truncated MyD88 polypeptide region lacking the TIR domain and a CD40 cytoplasmic polypeptide region lacking the CD40 extracellular domain.

18. The modified cell of claim 16, wherein the cell further comprises a third polynucleotide, wherein the third polynucleotide encodes a chimeric antigen receptor or a recombinant T cell receptor.

19.-22. (canceled)

23. The modified cell of claim 16, wherein the cell is a T cell, tumor infiltrating lymphocyte, NK-T cell, or NK cell.

24.-38. (canceled)

39. The modified cell of claim 1, wherein the chimeric pro-apoptotic polypeptide comprises a pro-apoptotic polypeptide region, a FKBP12-Rapamycin-Binding (FRB) domain polypeptide, or FRB variant polypeptide region; and the chimeric costimulating polypeptide comprises two FKBP12 variant polypeptide regions and a truncated MyD88 polypeptide region lacking the TIR domain.

40. The modified cell of claim 39, wherein the chimeric costimulating polypeptide comprises a CD40 cytoplasmic polypeptide region lacking the CD40 extracellular domain.

41. The modified cell of claim 39, wherein the FKBP12 variant comprises an amino acid substitution at amino acid residue 36.

42. The nucleic acid of claim 41, wherein the FKBP12 variant polypeptide region is a FKBP12v36 polypeptide region.

43. The nucleic acid of claim 39, wherein the FRB variant polypeptide region is selected from the group consisting of KLW (T2098L) (FRBL), KTF (W2101 F), and KLF (T2098L, W2101 F).

44. The modified cell of claim 39, comprising a polynucleotide that encodes a chimeric antigen receptor or a recombinant TCR.

45. The modified cell of claim 16, wherein the FKBP12 variant comprises an amino acid substitution at amino acid residue 36.

46. The modified cell of claim 45, wherein the FKBP12 variant polypeptide region is a FKBP12v36 polypeptide region.

47. The modified cell of claim 16, wherein the FRB variant polypeptide region is selected from the group consisting of KLW (T2098L) (FRBL), KTF (W2101 F), and KLF (T2098L, W2101 F).

48. The modified cell of claim 2, wherein the FKBP12 variant polypeptide regions are FKBP12v36 polypeptide regions, and the pro-apoptotic polypeptide region comprises a Caspase-9 polypeptide lacking the CARD domain.

49. The modified cell of claim 1, wherein in the chimeric pro-apoptotic polypeptide, the FRB polypeptide region or FRB variant polypeptide region, and FKBP12 polypeptide region, are located amino-terminal to the pro-apoptotic polypeptide region; and in the chimeric stimulating polypeptide, the two FKBP12 variant polypeptide regions are located amino-terminal to the costimulatory polypeptide region.

50. The modified cell of claim 16, wherein in the chimeric pro-apoptotic polypeptide, the FKBP12 variant polypeptide region is located amino-terminal to the pro-apoptotic polypeptide region; and in the chimeric stimulating polypeptide, the FRB polypeptide region or FRB variant polypeptide region, and FKBP12 polypeptide region, are located amino-terminal to the costimulatory polypeptide region.
